(19) United States
(12) Patent Application Publication
(10) Pub. No.: US 2023/0083973 A1
(43) Pub. Date: Mar. 16, 2023

(54) SUBSTITUTED CYCLOPROPYL-2,2'-BIPYRIMIDINYL COMPOUNDS, ANALOGUES THEREOF, AND METHODS USING SAME

(71) Applicant: Arbutus Biopharma, Inc., Warminster, PA (US)

(72) Inventors: Shuai Chen, Warminster, PA (US); Andrew G. Cole, Cranbury, NJ (US); Bruce D. Dorsey, Ambler, PA (US); Benjamin J. Dugan, Glen Mills, PA (US); Yi Fan, Doylestown, PA (US); Dimitar B. Gotchev, Hatboro, PA (US); Jorge Quintero, Cherry Hill, NJ (US); Michael J. Sofia, Doylestown, PA (US)

(21) Appl. No.: 17/629,188

(22) PCT Filed: Jul. 31, 2020
(86) PCT No.: PCT/US20/44405

§ 371 Date: Jan. 21, 2022

Related U.S. Application Data
(60) Provisional application No. 62/882,243, filed on Aug. 2, 2019.

(51) Int. Cl. C07D 401/04; A61K 45/06; A61K 31/444; C07D 403/04; A61K 31/506; C07D 403/14; C07D 491/107; C07D 417/14; C07D 401/14; C07D 413/14; A61K 31/5377; C07D 405/14; A61P 31/20

(52) U.S. Cl. CPC C07D 401/04; A61K 45/06; A61K 31/444; C07D 403/04; A61K 31/506; C07D 403/14; C07D 491/107; C07D 417/14; C07D 401/14; C07D 413/14; A61K 31/5377; C07D 405/14; A61P 31/20

(57) ABSTRACT

The present disclosure includes novel substituted cyclopropyl-2,2′-bipyrimidinyl compounds, and compositions comprising the same. In certain embodiments, the compounds of the disclosure can be used to treat or prevent hepatitis B virus (HBV) infection and/or hepatitis D virus (HDV) infection in a patient.

### CROSS-REFERENCE TO RELATED APPLICATIONS

This application claims priority under 35 U.S.C. § 119(e) to U.S. Provisional Application No. 62/882,243, filed Aug. 2, 2019, which is hereby incorporated by reference herein in its entirety.

### BACKGROUND

Hepatitis B is one of the world's most prevalent diseases. Although most individuals resolve the infection following acute symptoms, approximately 30% of cases become chronic. 350-400 million people worldwide are estimated to have chronic hepatitis B, leading to 0.5-1 million deaths per year, due largely to the development of hepatocellular carcinoma, cirrhosis, and/or other complications. Hepatitis B is caused by hepatitis B virus (HBV), a noncytopathic, liver tropic DNA virus belonging to Hepadnaviridae family.

A limited number of drugs are currently approved for the management of chronic hepatitis B, including two formulations of alpha-interferon (standard and pegylated) and five nucleoside/nucleotide analogues (lamivudine, adefovir, entecavir, telbivudine, and tenofovir) that inhibit HBV DNA polymerase. At present, the first-line treatment choices are entecavir, tenofovir, or peg-interferon alfa-2a. However, peg-interferon alfa-2a achieves desirable serological milestones in only one third of treated patients, and is frequently associated with severe side effects. Entecavir and tenofovir require long-term or possibly lifetime administration to continuously suppress HBV replication, and may eventually fail due to emergence of drug-resistant viruses.

HBV is an enveloped virus with an unusual mode of replication, centering on the establishment of a covalently closed circular DNA (cccDNA) copy of its genome in the host cell nucleus. Pregenomic (pg) RNA is the template for reverse transcriptional replication of HBV DNA. The encapsidation of pg RNA, together with viral DNA polymerase, into a nucleocapsid is essential for the subsequent viral DNA synthesis.

Aside from being a critical structural component of the virion, the HBV envelope is a major factor in the disease process. In chronically infected individuals, serum levels of HBV surface antigen (HBsAg) can be as high as 400 μg/ml, driven by the propensity for infected cells to secrete non-infectious subviral particles at levels far in excess of infectious (Dane) particles. HBsAg comprises the principal antigenic determinant in HBV infection and is composed of the small, middle and large surface antigens (S, M, and L, respectively). These proteins are produced from a single open reading frame as three separate N-glycosylated polypeptides through utilization of alternative transcriptional start sites (for L and M/S mRNAs) and initiation codons (for L, M, and S).

Although the viral polymerase and HBsAg perform distinct functions, both are essential proteins for the virus to complete its life cycle and be infectious. HBV lacking HBsAg is completely defective, and cannot infect or cause infection. HBsAg protects the virus nucleocapsid, begins the infectious cycle, and mediates morphogenesis and secretion of newly forming virus from the infected cell.

People chronically infected with HBV are usually characterized by readily detectable levels of circulating antibody specific to the viral capsid (HBc), with little, if any detectable levels of antibody to HBsAg. There is evidence that chronic carriers produce antibodies to HBsAg, but these antibodies are complexed with the circulating HBsAg, which can be present in mg/mL amounts in a chronic carrier's circulation. Reducing the amount of circulating levels of HBsAg might allow any present anti-HBsA to manage the infection. Further, even if nucleocapsids free of HBsAg were to be expressed or secreted into circulation (perhaps as a result of cell death), the high levels of anti-HBc would quickly complex with them and result in their clearance.

Studies have shown that the presence of subviral particles in a culture of infected hepatocytes may have a transactivating function on viral genomic replication, and the circulating surface antigen suppresses virus-specific immune response. Furthermore, the scarcity of virus-specific cytotoxic T lymphocytes (CTLs), that is a hallmark of chronic HBV infection, may be due to repression of MHC I presentation by intracellular expression of L and M in infected hepatocytes. Existing FDA-approved therapies do not significantly affect HBsAg serum levels.

Hepatitis D virus (HDV) is a small circular enveloped RNA virus that can propagate only in the presence of HBV. In particular, HDV requires the HBV surface antigen protein to propagate itself. Infection with both HBV and HDV results in more severe complications compared to infection with HBV alone. These complications include a greater likelihood of experiencing liver failure in acute infections and a rapid progression to liver cirrhosis, with an increased chance of developing liver cancer in chronic infections. In combination with hepatitis B virus, hepatitis D has the highest mortality rate of all the hepatitis infections. The routes of transmission of HDV are similar to those for HBV. Infection is largely restricted to persons at high risk of HBV infection, particularly injecting drug users and persons receiving clotting factor concentrates.

Currently, there is no effective antiviral therapy available for the treatment of acute or chronic type D hepatitis. Interferon-alfa, given weekly for 12 to 18 months, is the only licensed treatment for hepatitis D. Response to this therapy is limited-in only about one-quarter of patients is serum HDV RNA undetectable 6 months post therapy.

There is thus a need in the art for novel compounds and/or compositions that can be used to treat and/or prevent HBV and/or HBV-HDV infection in a subject. In certain embodiments, the compounds can be used in patients that are HBV and/or HBV-HDV infected, patients who are at risk of becoming HBV and/or HBV-HDV infected, and/or patients that are infected with drug-resistant HBV and/or HDV. The present disclosure addresses this need.

### BRIEF SUMMARY OF THE DISCLOSURE

The disclosure provides a compound of formula (I) or (II) or (III), or a salt, solvate, geometric isomer, stereoisomer, tautomer, and any mixtures thereof:

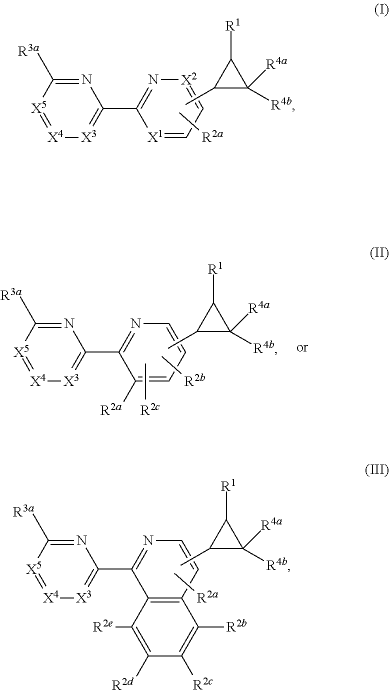

wherein R¹, R^{2a}—R^{2e}, R^{3a}, R^{4a}—R^{4b}, and X¹-X⁵ are defined elsewhere herein. The present disclosure further provides a pharmaceutical composition comprising at least one compound of the disclosure and at least one pharmaceutically acceptable carrier. The present disclosure further provides a method of treating, ameliorating, and/or preventing hepatitis virus infection in a subject, the method comprising administering to the subject in need thereof a therapeutically effective amount of at least one compound and/or pharmaceutical composition of the invention.

### DETAILED DESCRIPTION OF THE DISCLOSURE

The disclosure relates, in certain aspects, to the discovery of certain substituted polyaromatic compounds that are useful to treat and/or prevent HBV and/or HBV-HDV infection and related conditions in a subject. In certain embodiments, the compounds inhibit and/or reduce HBsAg secretion in an HBV-infected and/or HBV-HDV-infected subject. In other embodiments, the compounds reduce or minimize levels of HBsAg in an HBV-infected and/or HBV-HDV-infected subject. In yet other embodiments, the compounds reduce or minimize levels of HBeAg in an HBV-infected and/or HBV-HDV-infected subject. In yet other embodiments, the compounds reduce or minimize levels of hepatitis B core protein in an HBV-infected and/or HBV-HDV-infected subject. In yet other embodiments, the compounds reduce or minimize levels of pg RNA in an HBV-infected and/or HBV-HDV-infected subject.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, separation science, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

As used herein, the term “alkenyl,” employed alone or in combination with other terms, means, unless otherwise stated, a stable monounsaturated or diunsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkoxy” employed alone or in combination with other terms means, unless otherwise stated, an alkyl group having the designated number of carbon atoms, as defined elsewhere herein, connected to the rest of the molecule via an oxygen atom, such as, for example, methoxy, ethoxy, 1-propoxy, 2-propoxy (or isopropoxy) and the higher homologs and isomers. A specific example is (C₁-C₃)alkoxy, such as, but not limited to, ethoxy and methoxy.

As used herein, the term “alkyl” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tent-butyl, pentyl, neopentyl, hexyl, and cyclopropylmethyl. A specific embodiment is (C₁-C₆)alkyl, such as, but not limited to, ethyl, methyl, isopropyl, isobutyl, n-pentyl, n-hexyl, and cyclopropylmethyl.

As used herein, the term “alkynyl” employed alone or in combination with other terms means, unless otherwise stated, a stable straight chain or branched chain hydrocarbon group with a triple carbon-carbon bond, having the stated number of carbon atoms. Non-limiting examples include ethynyl and propynyl, and the higher homologs and isomers. The term “propargylic” refers to a group exemplified by —CH₂—C≡CH. The term “homopropargylic” refers to a group exemplified by —CH₂—C≡CH.

As used herein, the term “aromatic” refers to a carbocycle or heterocycle with one or more polyunsaturated rings and having aromatic character, i.e., having (4n+2) delocalized 7C (pi) electrons, where ‘n’ is an integer.

As used herein, the term “aryl” employed alone or in combination with other terms means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl and naphthyl. Aryl groups also include, for example, phenyl or naphthyl rings fused with one or more saturated or partially saturated carbon rings (e.g., bicyclo[4.2.0]octa-1,3,5-trienyl, or indanyl), which can be substituted at one or more carbon atoms of the aromatic and/or saturated or partially saturated rings.

As used herein, the term “aryl-(C₁-C₆)alkyl” refers to a functional group wherein a one-to-six carbon alkylene chain is attached to an aryl group, e.g., —CH₂CH₂-phenyl or —CH₂-phenyl (or benzyl). Specific examples are aryl-CH₂— and aryl-CH(CH₃)—. The term “substituted aryl-(C₁-C₆)alkyl” refers to an aryl-(C₁-C₆)alkyl functional group in which the aryl group is substituted. A specific example is substituted aryl(CH₂)—. Similarly, the term “heteroaryl-(C₁-C₆)alkyl” refers to a functional group wherein a one-to-three carbon alkylene chain is attached to a heteroaryl group, e.g., —CH₂CH₂-pyridyl. A specific example is heteroaryl-(CH₂)—. The term “substituted heteroaryl-(C₁-C₆)alkyl” refers to a heteroaryl-(C₁-C₆)alkyl functional group in which the heteroaryl group is substituted. A specific example is substituted heteroaryl-(CH₂)—.

In one aspect, the terms “co-administered” and “co-administration” as relating to a subject refer to administering to the subject a compound and/or composition of the disclosure along with a compound and/or composition that may also treat or prevent a disease or disorder contemplated herein. In certain embodiments, the co-administered compounds and/or compositions are administered separately, or in any kind of combination as part of a single therapeutic approach. The co-administered compound and/or composition may be formulated in any kind of combinations as mixtures of solids and liquids under a variety of solid, gel, and liquid formulations, and as a solution.

As used herein, the term “cycloalkyl” by itself or as part of another substituent refers to, unless otherwise stated, a cyclic chain hydrocarbon having the number of carbon atoms designated (i.e., C₃-C₆ refers to a cyclic group comprising a ring group consisting of three to six carbon atoms) and includes straight, branched chain or cyclic substituent groups. Examples of (C₃-C₆)cycloalkyl groups are cyclopropyl, cyclobutyl, cyclopentyl and cyclohexyl. Cycloalkyl rings can be optionally substituted. Non-limiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes bicyclic hydrocarbon rings, non-limiting examples of which include, bicyclo[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

As used herein, the term “halide” refers to a halogen atom bearing a negative charge. The halide anions are fluoride (F⁻), chloride (Cl⁻), bromide (Br⁻), and iodide (I⁻).

As used herein, the term “halo” or “halogen” alone or as part of another substituent refers to, unless otherwise stated, a fluorine, chlorine, bromine, or iodine atom.

As used herein, the term “heteroalkenyl” by itself or in combination with another term refers to, unless otherwise stated, a stable straight or branched chain monounsaturated or diunsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, and —CH₂—CH═CH—CH₂—SH.

As used herein, the term “heteroalkyl” by itself or in combination with another term refers to, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —OCH₂CH₂CH₃, —CH₂CH₂CH₂OH, —CH₂CH₂NHCH₃, —CH₂SCH₂CH₃, and —CH₂CH₂S(═O)CH₃. Up to two heteroatoms may be consecutive, such as, for example, —CH₂NH—OCH₃, or —CH₂CH₂SSCH₃.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

As used herein, the term “heterocycle” or “heterocyclyl” or “heterocyclic” by itself or as part of another substituent refers to, unless otherwise stated, an unsubstituted or substituted, stable, mono- or multi-cyclic heterocyclic ring system that comprises carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain embodiments, the heterocycle is a heteroaryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl, and 1,3,4-oxadiazolyl.

Examples of polycyclic heterocycles include indolyl (such as, but not limited to, 3-, 4-, 5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “pharmaceutical composition” or “composition” refers to a mixture of at least one compound useful within the disclosure with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the disclosure, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface-active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the subject. Supplementary active compounds may also be incorporated into the compositions. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compound prepared from pharmaceutically acceptable non-toxic acids and/or bases, including inorganic acids, inorganic bases, organic acids, inorganic bases, solvates (including hydrates) and clathrates thereof.

As used herein, a “pharmaceutically effective amount,” “therapeutically effective amount,” or “effective amount” of a compound is that amount of compound that is sufficient to provide a beneficial effect to the subject to which the compound is administered.

The term “prevent,” “preventing,” or “prevention” as used herein means avoiding or delaying the onset of symptoms associated with a disease or condition in a subject that has not developed such symptoms at the time the administering of an agent or compound commences. Disease, condition and disorder are used interchangeably herein.

By the term “specifically bind” or “specifically binds” as used herein is meant that a first molecule preferentially binds to a second molecule (e.g., a particular receptor or enzyme), but does not necessarily bind only to that second molecule.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In certain embodiments, the subject is human.

As used herein, the term “substituted” refers to that an atom or group of atoms has replaced hydrogen as the substituent attached to another group.

As used herein, the term “substituted alkyl,” “substituted cycloalkyl,” “substituted alkenyl,” or “substituted alkynyl” refers to alkyl, cycloalkyl, alkenyl, or alkynyl, as defined elsewhere herein, substituted by one, two or three substituents independently selected from the group consisting of halogen, —OH, alkoxy, tetrahydro-2-H-pyranyl, —NH₂, —NH(C₁-C₆ alkyl), —N(C₁-C₆ alkyl)₂, 1-methyl-imidazol-2-yl, pyridin-2-yl, pyridin-3-yl, pyridin-4-yl, —C(═O)OH, —C(═O)O(C₁-C₆)alkyl, trifluoromethyl, —C(═O)NH₂, —C(═O)NH((C₁-C₆)alkyl, —C(═O)N((C₁-C₆)alkyl)₂, —SO₂NH₂, —SO₂NH(C₁-C₆ alkyl), —SO₂N(C₁-C₆ alkyl)₂, —C(═NH)NH₂, and —NO₂, in certain embodiments containing one or two substituents independently selected from halogen, —OH, alkoxy, —NH₂, trifluoromethyl, —N(CH₃)₂, and —C(═O)OH, in certain embodiments independently selected from halogen, alkoxy and —OH. Examples of substituted alkyls include, but are not limited to, 2,2-difluoropropyl, 2-carboxycyclopentyl and 3-chloropropyl.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In other embodiments, the substituents vary in number between one and three. In yet another embodiments, the substituents vary in number between one and two. In yet other embodiments, the substituents are independently selected from the group consisting of C₁-C₆ alkyl, —OH, C₁-C₆ alkoxy, halogen, cyano, amino, acetamido and nitro. As used herein, where a substituent is an alkyl or alkoxy group, the carbon chain may be branched, straight or cyclic.

Unless otherwise noted, when two substituents are taken together to form a ring having a specified number of ring atoms (e.g., R² and R³ taken together with the nitrogen to which they are attached to form a ring having from 3 to 7 ring members), the ring can have carbon atoms and optionally one or more (e.g., 1 to 3) additional heteroatoms independently selected from nitrogen, oxygen, or sulfur. The ring can be saturated or partially saturated, and can be optionally substituted.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given elsewhere herein for “alkyl” and “aryl” respectively.

In certain embodiments, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The terms “treat,” “treating” and “treatment,” as used herein, means reducing the frequency or severity with which symptoms of a disease or condition are experienced by a subject by virtue of administering an agent or compound to the subject.

The following non-limiting abbreviations are used herein: cccDNA, covalently closed circular DNA; CH₂Cl₂, methylene chloride; DMF, dimethylformamide; EtOAc, ethyl acetate; HBc, hepatitis B capsid; HBV, hepatitis B virus; HDV, hepatitis D virus; HBeAg, hepatitis B e-antigen; HBsAg, hepatitis B virus surface antigen; HPLC, high-performance liquid chromatography; IPA, isopropyl alcohol; MeOH, methanol; pg RNA, pregenomic RNA; SiO₂, silica; THF, tetrahydrofuran.

Ranges: throughout this disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise. This applies regardless of the breadth of the range.

### Compounds

The disclosure includes certain compounds recited herein, as well as any salt, solvate, geometric isomer (such as, in a non-limiting example, any geometric isomer and any mixtures thereof, such as, in a non-limiting example, mixtures in any proportions of any geometric isomers thereof), stereoisomer (such as, in a non-limiting example, any enantiomer or diastereoisomer, and any mixtures thereof, such as, in a non-limiting example, mixtures in any proportions of any enantiomers and/or diastereoisomers thereof), tautomer (such as, in a non-limiting example, any tautomer and any mixtures thereof, such as, in a non-limiting example, mixtures in any proportions of any tautomers thereof), and any mixtures thereof.

The disclosure includes a compound of formula (I) or (II) or (III), or a salt, solvate, geometric isomer, stereoisomer, tautomer, and any mixtures thereof:

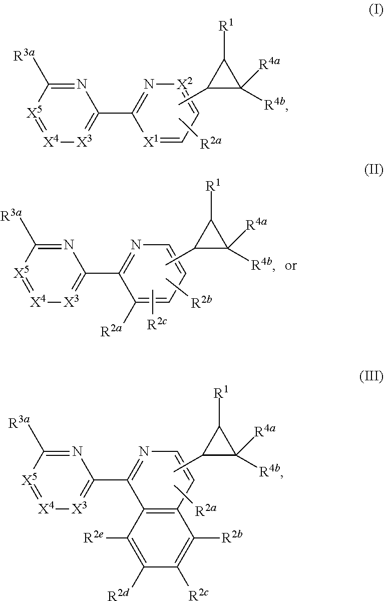

wherein:
one of the following applies:
   (i) X¹ is N, X² is CR^{2b}, or
   (ii) X¹ is CR^{2c}, X² is N;
one of the following applies:
   (i) X³ is N, X⁴ is CR^{3c}, X⁵ is CR^{3d}; or
   (ii) X³ is CR^{3b}, X⁴ is N, X⁵ is CR^{3d}; or
   (iii) X³ is CR^{3b}, X⁴ is CR^{3c}, X⁵ is N;
R¹ is selected from the group consisting of optionally substituted phenyl, optionally substituted naphthyl, optionally substituted pyridinyl, optionally substituted pyrimidinyl, optionally substituted benzo[d]thiazolyl; optionally substituted benzoimidazolyl, optionally substituted imidazo[1,2-a]pyridinyl, optionally substituted quinolinyl, optionally substituted isoquinolinyl, optionally substituted 1H-indazolyl, and optionally substituted 2H-indazolyl;
each occurrence of R^{2a}, R^{2b}, R^{2c}, R^{2d}, and R^{2e} is independently selected from the group consisting of H, halogen, cyano, nitro, optionally substituted C₁-C₆ alkyl, optionally substituted C₁-C₆ haloalkyl, optionally substituted C₃-C₈ cycloalkyl, optionally substituted C₁-C₆ haloalkoxy, optionally substituted C₁-C₆ hydroxyalkyl, —OR′, —SR′, —S(═O)R′, —S(O)₂R′, —N(R′)(R′), —N(R′)C(═O)(R′), —C(═O)N(R′)(R′), optionally substituted phenyl, optionally substituted heterocyclyl, and optionally substituted heteroaryl,
   wherein each occurrence of R′ is independently selected from the group consisting of H, optionally substituted C₁-C₆ alkyl, and optionally substituted C₃-C₈ cycloalkyl;
each occurrence of R^{3a}, R^{3b}, R^{3c}, and R^{3d} is independently selected from the group consisting of H, halogen, cyano, nitro, optionally substituted C₁-C₆ alkyl, optionally substituted C₁-C₆ haloalkyl, optionally substituted C₃-C₈ cycloalkyl, optionally substituted C₁-C₆ haloalkoxy, optionally substituted C₁-C₆ hydroxyalkyl, —OR″, -SR″, —S(═O)R″, —S(O)₂R″, —N(R″)(R″), optionally substituted phenyl, optionally substituted heterocyclyl, and optionally substituted heteroaryl,
   wherein each occurrence of R″ is independently selected from the group consisting of H, optionally substituted C₁-C₆ alkyl, and optionally substituted C₃-C₈ cycloalkyl;
R^{4a} is selected from the group consisting of R^{4c}, F, Cl, Br, I, —OR^{4c}, and —C(═O)OR^{4c}, wherein each occurrence of R^{4d} is independently H, optionally substituted C₁-C₆ alkyl, or optionally substituted C₃-C₈ cycloalkyl, and
R^{4b} is selected from the group consisting of R^{4d}, F, Cl, Br, I, —OR^{4d}, and —C(═O)OR^{4d}, wherein each occurrence of R^{4d} is independently H, optionally substituted C₁-C₆ alkyl, or optionally substituted C₃-C₈ cycloalkyl.

In certain embodiments, X¹ is N and X² is CR^{2b}. In certain embodiments, X¹ is CR^{2c} and X² is N. In certain embodiments, X³ is N, X⁴ is CR^{3b}, and X⁵ is CR^{3d}. In certain embodiments, X³ is CR^{3b}, X⁴ is N, and X⁵ is CR^{3d}. In certain embodiments, X³ is CR^{3b}, X⁴ is CR^{3c}, and X⁵ is N.

In certain embodiments, R¹ is unsubstituted phenyl. In certain embodiments, R¹ is substituted phenyl. In certain embodiments, R¹ is unsubstituted naphthyl. In certain embodiments, R¹ is substituted naphthyl. In certain embodiments, R¹ is unsubstituted pyridinyl. In certain embodiments, R¹ is substituted pyridinyl. In certain embodiments, R¹ is unsubstituted pyrimidinyl. In certain embodiments, R¹ is substituted pyrimidinyl. In certain embodiments, R¹ is unsubstituted benzo[d]thiazolyl. In certain embodiments, R¹ is substituted benzo[d]thiazolyl. In certain embodiments, R¹ is unsubstituted benzoimidazolyl. In certain embodiments, R¹ is substituted benzoimidazolyl. In certain embodiments, R¹ is unsubstituted imidazo[1,2-a]pyridinyl. In certain embodiments, R¹ is substituted imidazo[1,2-a]pyridinyl. In certain embodiments, R¹ is unsubstituted quinolinyl. In certain embodiments, R¹ is substituted quinolinyl. In certain embodiments, R¹ is unsubstituted isoquinolinyl. In certain embodiments, R¹ is substituted isoquinolinyl. In certain embodiments, R¹ is unsubstituted 1H-indazolyl. In certain embodiments, R¹ is substituted 1H-indazolyl. In certain embodiments, R¹ is unsubstituted 2H-indazolyl. In certain embodiments, R¹ is substituted 2H-indazolyl.

In certain embodiments, R¹ is substituted with at least one selected from the group consisting of H, F, Cl, Br, I, optionally substituted C₁-C₆ alkyl, optionally substituted C₁-C₆ haloalkyl, optionally substituted C₁-C₆ alkoxy, optionally substituted C₁-C₆ haloalkoxy, optionally substituted phenyl, —NR″′R″′, —C(═O)NR″′R″′, —NHC(═O)R″′, and optionally substituted heterocyclyl; wherein each occurrence of R″′ is independently selected from the group consisting of H, optionally substituted C₁-C₆ alkyl, optionally substituted C₃-C₈ cycloalkyl, and optionally substituted heterocyclyl. In certain embodiments, R¹ is substituted with at least one selected from the group consisting of H; F; Cl; Br; I; C₁-C₆ alkyl; C₁-C₆ alkyl substituted with at least one of F, Cl, Br, I, OH, CN, C₁-C₆ alkyl, C₃-C₈ cycloalkyl, C₁-C₆ alkoxy, and C₃-C₈ cycloalkoxy; C₁-C₆ alkoxy; C₁-C₆ alkoxy substituted with at least one of F, Cl, Br, I, OH, CN, C₁-C₆ alkyl, C₃-C₈ cycloalkyl, C₁-C₆ alkoxy, and C₃-C₈ cycloalkoxy; C₃-C₈ cycloalkyl; C₃-C₈ cycloalkyl substituted with at least one of F, Cl, Br, I, OH, CN, C₁-C₆ alkyl, C₃-C₈ cycloalkyl, C₁-C₆ alkoxy, and C₃-C₈ cycloalkoxy; C₃-C₈ cycloalkoxy; C₃-C₈ cycloalkoxy substituted with at least one of F, Cl, Br, I, OH, CN, C₁-C₆ alkyl, C₃-C₈ cycloalkyl, C₁-C₆ alkoxy, and C₃-C₈ cycloalkoxy; —NH₂; —NH(C₁-C₆ alkyl); —N(C₁-C₆ alkyl)(C₁-C₆ alkyl); —C(═O)NH(C₁-C₆ alkyl); —NHC(═O)H; —NHC(═O)(C₁-C₆ alkyl); optionally substituted phenyl; optionally substituted spiroheterocyclyl; optionally substituted pyrrolidinonyl; optionally substituted azetidinyl; optionally substituted pyrrolidinyl; optionally substituted pyrrolidinonyl; optionally substituted piperidinyl; optionally substituted piperazinyl; optionally substituted morpholinyl; optionally substituted pyrrolyl; optionally substituted pyrazolyl; optionally substituted imidazolyl; optionally substituted 1H-benzo[d]imidazyl; optionally substituted indazolyl; optionally substituted benzo[d]thiazolyl; and optionally substituted 1H-benzo[d]imidazolyl.

In certain embodiments, R¹ is substituted with at least one selected from the group consisting of H; F, Cl, Br, I, methyl, difluoromethyl, trifluoromethyl, ethyl, propyl, isopropyl, phenyl, methoxy, ethoxy, propoxy, isopropoxy, 2-methoxyethoxy, 3-methoxypropoxy, cyclopropylmethoxy, 2,2-difluoroethoxy, difluoromethoxy, trifluoromethoxy, (1-methyl-1H-1,2,4-triazol-3-yl)methoxy, (thiazol-2-yl)methoxy, (1-methyl-1H-pyrazol-3-yl)methoxy, 3-N-morpholinyl-propoxy, tetrahydrofuranoxy, dimethylamino, diethylamino, N-2-hydroxyethylamino, N-methyl-N-2-hydroxyethylamino, cyclopropylamino, cyclobutylamino, cyclopentylamino, cyclohexylamino, N-methylpyrrolidinyl-methylamino, N-acetylpyrrolidinyl-methylamino, (N-methyl)(N-methyl-oxet-3-yl)amino, dimethylamino, pyrrolidin-2-on-1-yl, azetidinyl, 3,3-dimethylazetidinyl, 3-hydroxy-azetidinyl, 3-methoxy-azetidinyl, 3-ethoxy-azetidinyl, 3-propoxy-azetidinyl, 3-isopropoxy-azetidinyl, 3-(2-methoxyethoxy)azetidinyl, 3-(tert-butylsulfonyl)azetidinyl, 3-(optionally substituted phenyl)azetidinyl, pyrrolidinyl, pyrrolidin-2-one-1-yl, 3-hydroxypyrrolidinyl, 3-methoxypyrrolidinyl, 3,3-difluoropyrrolidinyl, 3,4-dihydroxypyrrolidin-1-yl, 3,4-dimethoxypyrrolidin-1-yl, piperidinyl, piperazinyl, morpholinyl, 1H-pyrrol-1-yl, 3,4-difluoro-1H-pyrrol-1-yl, pyrazol-1-yl, 4-methyl-1H-pyrazol-1-yl, 4-methoxy-1H-pyrazol-1-yl, 4-hydroxymethyl-1H-pyrazol-1-yl, 4-methoxymethyl-1H-pyrazol-1-yl, 1H-imidazol-1-yl, methyl-1H-imidazol-1-yl, N4-(C₁-C₆ alkyl)-piperazin-1-yl (such as but not limited to N4-methyl-piperazin-1-yl), N4-[SO₂(C₁-C₆ alkyl)]-piperazin-1-yl (such as but not limited to N4-SO₂CH₃-piperazin-1-yl), imidazolyl, —C(═O)NH₂, —C(═O)NHCH₃, —C(═O)N(CH₃)₂, —NHC(═O)CH₃, 2-oxa-6-azaspiro[3.3]hept-6-yl, 2-oxa-6-azaspiro[3.3]hept-6-oxy, 2-azaspiro[3.3]hept-2-yl, 6-hydroxy-2-azaspiro[3.3]hept-2-yl, 2-oxaspiro[3.3]hept-6-yl, 2-oxa-6-azaspiro[3.4]oct-6-yl, 7-oxa-2-azaspiro[3.5]non-2-yl, 5,6-dimethoxy-1H-benzo[d]imidazyl, 1H-indazolyl, 6,7-difluoro-1-(3-methoxypropyl)-1H-indazolyl, 4-fluoro-2-(3-methoxypropyl)-2H-indazolyl, 7-fluoro-1-(3-methoxypropyl)-1H-indazolyl, 7-fluoro-2-(3-methoxypropyl)-2H-indazolyl, 4-fluoro-1-(3-methoxypropyl)-1H-indazolyl, benzo[d]thiazolyl, 2-methylbenzo[d]thiazolyl, 4,6-difluorobenzo[d]thiazolyl, 4-fluoro-2-methylbenzo[d]thiazolyl, 1H-benzo[d]imidazolyl, 4-fluoro-1-isopropyl-2-methyl-1H-benzo[d]imidazolyl, 7-fluoro-1-(3-methoxypropyl)-1H-benzo[d]imidazolyl, 4-fluoro-1-(3-methoxypropyl)-1H-benzo[d]imidazolyl.

In certain embodiments, R¹ is selected from the group consisting of: 3-azetidin-1-yl-4-chloro-phenyl; 3-acetylamino-4-fluoro-phenyl; 3-acetylamino-4-chloro-phenyl; 3-azetidin-1-yl-4-fluoro-phenyl; 2-chloro-4-methoxy-phenyl; 3-chloro-4-fluoro-5-(pyrrolidin-1-yl)-phenyl; 3-chloro-4-fluoro-5-(3-hydroxypyrrolidin-1-yl)-phenyl; 3-chloro-4-fluoro-5-(3-methoxypyrrolidin-1-yl)-phenyl; 3-chloro-4-fluoro-phenyl; 3-chloro-4-methoxy-phenyl; 4-chloro-3-fluoro-5-(pyrrolidin-1-yl)-phenyl; 4-chloro-3,5-dimethoxy-phenyl; 4-chloro-3-fluoro-5-methoxy-phenyl; 4-chloro-5-methoxy-3-methyl-phenyl; 4-chloro-5-methoxy-2-phenyl-phenyl; 4-chloro-3-trifluoromethoxy-phenyl; 4-chloro-3-(4-methylsulfonylpiperazin-1-yl)-phenyl; 4-chloro-3-(morpholin-1-yl)-phenyl; 4-chloro-3-(pyrrolidin-2-on-1-yl)-phenyl; 4-chloro-3-(pyrrolidin-1-yl)-phenyl; 4-chloro-3-(3,3-difluoro-pyrrolidin-1yl)-phenyl; 4-chloro-3-difluoromethoxy-phenyl; 4-chloro-3-(2-difluoroethoxy)-phenyl; 4-chloro-3-(4-methylpiperazin-1-yl)-phenyl; 4-chloro-2-methyl-5-methoxy-phenyl; 4-chloro-3-methoxy-2-methyl-phenyl; 4-chloro-5-methoxy-2-(N-methylaminocarbonyl)-phenyl; 4-chloro-3-cyclopropylmethoxy-5-methyl-phenyl; 4-chloro-3-cyclopropylmethoxy-2-methyl-phenyl; 4-chloro-2,3-dimethoxy-phenyl; 4-chloro-2-fluoro-5-methoxy-phenyl; 4-chloro-2-fluoro-3-methoxy-phenyl; 4-chloro-3-methoxy-phenyl; 4-chloro-6-ethyl-3-methoxy-phenyl; 4-chloro-3-methoxy-6-propyl-phenyl; 4-chloro-3-dimethylamino-phenyl; 4-chloro-3-dimethylaminocarbonyl-phenyl; 5-chloro-4-fluoro-2-(2-methoxy-ethoxy)-phenyl; 5-chloro-4-fluoro-2-(3-methoxy-propoxy)-phenyl; 2,4-dichloro-3-methoxy-phenyl; 2,4-dichloro-5-methoxy-phenyl; 2,4-difluoro-3-methoxy-phenyl; 3,4-difluoro-5-(pyrrolidin-1-yl)-phenyl; 3,4-difluoro-5-(3-hydroxypyrrolidin-1-yl)-phenyl; 3,4-difluoro-5-(3-methoxypyrrolidin-1-yl)-phenyl; 3,4-dichloro-5-methoxy-phenyl; 3,4-dichloro-phenyl; 3-(2-difluoroethoxy)-4-fluoro-phenyl; 3-difluoromethoxy-4-fluoro-phenyl; 3,4-difluoro-phenyl; 3,4-difluoro-5-methoxy-phenyl; 3-(3,3-difluoropyrrolidin-1yl)-4-fluoro-phenyl; 3-dimethylamino-4-fluoro-phenyl; 3-dimethylaminocarbonyl-4-fluoro-phenyl; 3,4-dimethoxy-phenyl; 5,6-dimethoxy-pyridin-3-yl; 6-ethyl-4-fluoro-3-methoxy-phenyl; 2-fluoro-4-methoxy-phenyl; 3-fluoro-4-(2-methoxy-ethoxy)-phenyl; 3-fluoro-4-methoxy-phenyl; 4-fluoro-3-(isopropylmethoxy)-phenyl; 4-fluoro-3-(2-methoxy-ethoxy)-phenyl; 4-fluoro-5-methoxy-2-phenyl-phenyl; 4-fluoro-3-methoxy-5-(pyrrolidin-1-yl)-phenyl; 4-fluoro-3-(morpholin-1-yl)-phenyl; 4-fluoro-3-piperidin-1-yl-phenyl; 4-fluoro-3-(pyrrolidin-2-on-1-yl)-phenyl; 4-fluoro-3-(pyrrolidin-1-yl)-phenyl; 4-fluoro-3-trifluoromethoxy-phenyl; 4-fluoro-3-(4-methylpiperazin-1-yl)-phenyl; 4-fluoro-3-(4-methylsulfonyl)piperazin-1-yl)-phenyl; 4-fluoro-3-(3-hydroxyazetidin-1-yl)-phenyl; 4-fluoro-3-(3-methoxyazetidin-1-yl)-phenyl; 4-fluoro-3-(3-hydroxy-pyrrolidin-1-yl)-phenyl; 4-fluoro-3-(3-methoxypyrrolidin-1-yl)-phenyl;4-fluoro-3-methoxy-phenyl; 4-fluoro-3-methoxy-6-propyl-phenyl; and 2-methylbenzo[d]thiazol-5-yl.

In certain embodiments, R¹ is

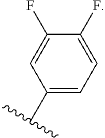 [SMILES: *c1ccc(F)c(F)c1]

In certain embodiments, R¹ is

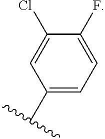 [SMILES: *c1ccc(F)c(Cl)c1]

In certain embodiments, R¹ is

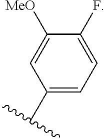 [SMILES: *c1ccc(F)c(OC)c1]

In certain embodiments, R¹ is

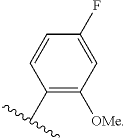 [SMILES: *c1ccc(F)cc1OC]

In certain embodiments, R¹ is

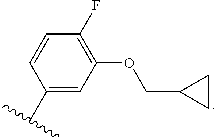 [SMILES: *c1ccc(F)c(OCC2CC2)c1]

In certain embodiments, R¹ is

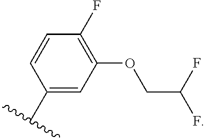 [SMILES: *c1ccc(F)c(OCC(F)F)c1]

In certain embodiments, R¹ is

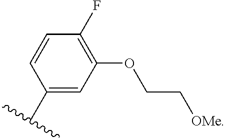 [SMILES: *c1ccc(F)c(OCCOC)c1]

In certain embodiments, R¹ is

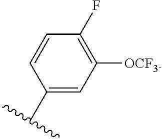 [SMILES: *c1ccc(F)c(OC(F)(F)F)c1]

In certain embodiments, R¹ is

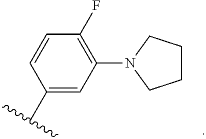 [SMILES: *c1ccc(F)c(N2CCCC2)c1]

In certain embodiments, R¹ is

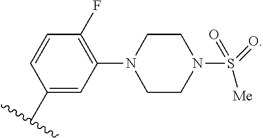 [SMILES: *c1ccc(F)c(N2CCN(S(C)(=O)=O)CC2)c1]

In certain embodiments, R¹ is

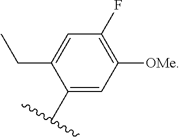 [SMILES: *c1cc(OC)c(F)cc1CC]

In certain embodiments, R¹ is

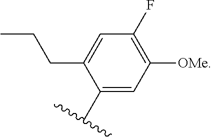 [SMILES: *c1cc(OC)c(F)cc1CCC]

In certain embodiments, R¹ is

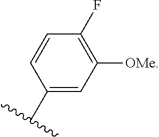 [SMILES: *c1ccc(F)c(OC)c1]

In certain embodiments, R¹ is

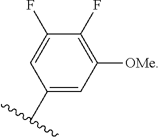 [SMILES: *c1cc(F)c(F)c(OC)c1]

In certain embodiments, R¹ is

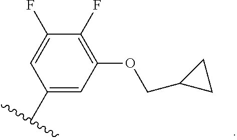 [SMILES: *c1cc(F)c(F)c(OCC2CC2)c1]

In certain embodiments, R¹ is

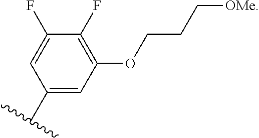 [SMILES: *c1cc(F)c(F)c(OCCCOC)c1]

In certain embodiments, R¹ is

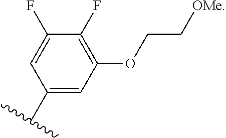 [SMILES: *c1cc(F)c(F)c(OCCOC)c1]

In certain embodiments, R¹ is

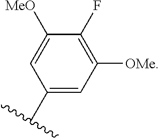 [SMILES: *c1cc(OC)c(F)c(OC)c1]

In certain embodiments, R¹ is

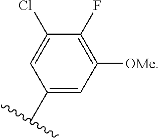 [SMILES: *c1cc(Cl)c(F)c(OC)c1]

In certain embodiments, R¹ is

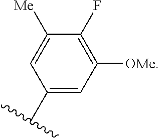 [SMILES: *c1cc(C)c(F)c(OC)c1]

In certain embodiments, R¹ is

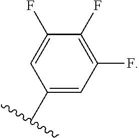 [SMILES: *c1cc(F)c(F)c(F)c1]

In certain embodiments, R¹ is

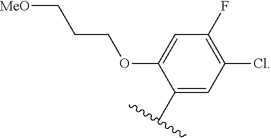 [SMILES: *c1cc(Cl)c(F)cc1OCCCOC]

In certain embodiments, R¹ is

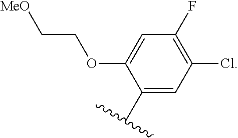 [SMILES: *c1cc(Cl)c(F)cc1OCCOC]

In certain embodiments, R¹ is

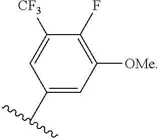 [SMILES: *c1cc(OC)c(F)c(C(F)(F)F)c1]

In certain embodiments, R¹ is

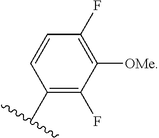 [SMILES: *c1ccc(F)c(OC)c1F]

In certain embodiments, R¹ is

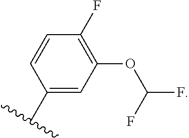 [SMILES: *c1ccc(F)c(OC(F)F)c1]

In certain embodiments, R¹ is

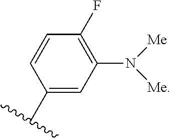 [SMILES: *c1ccc(F)c(N(C)C)c1]

In certain embodiments, R¹ is

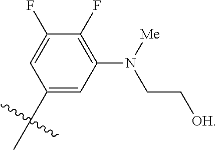 [SMILES: *c1cc(F)c(F)c(N(C)CCO)c1]

In certain embodiments, R¹ is

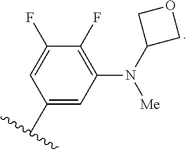 [SMILES: *c1cc(F)c(F)c(N(C)C2COC2)c1]

In certain embodiments, R¹ is

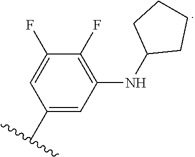 [SMILES: *c1cc(F)c(F)c(NC2CCCC2)c1]

In certain embodiments, R¹ is

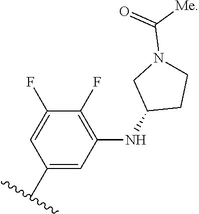 [SMILES: *c1cc(F)c(F)c(N[C@H]2CCN(C(C)=O)C2)c1]

In certain embodiments, R¹ is

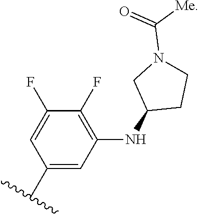 [SMILES: *c1cc(F)c(F)c(N[C@@H]2CCN(C(C)=O)C2)c1]

In certain embodiments, R¹ is

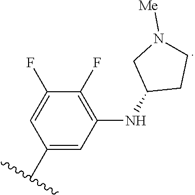 [SMILES: *c1cc(F)c(F)c(N[C@H]2CCN(C)C2)c1]

In certain embodiments, R¹ is

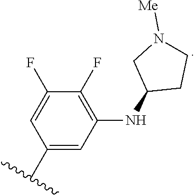 [SMILES: *c1cc(F)c(F)c(N[C@@H]2CCN(C)C2)c1]

In certain embodiments, R¹ is

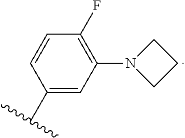 [SMILES: *c1ccc(F)c(N2CCC2)c1]

In certain embodiments, R¹ is

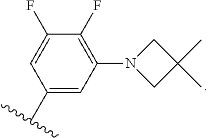 [SMILES: *c1cc(F)c(F)c(N2CC(C)(C)C2)c1]

In certain embodiments, R¹ is

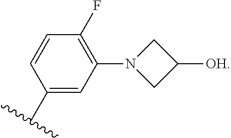 [SMILES: *c1ccc(F)c(N2CC(O)C2)c1]

In certain embodiments, R¹ is

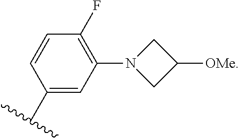 [SMILES: *c1ccc(F)c(N2CC(OC)C2)c1]

In certain embodiments, R¹ is

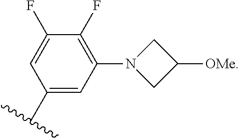 [SMILES: *c1cc(F)c(F)c(N2CC(OC)C2)c1]

In certain embodiments, R¹ is

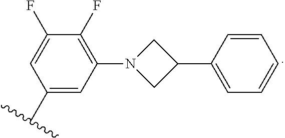 [SMILES: *c1cc(F)c(F)c(N2CC(c3ccccc3)C2)c1]

In certain embodiments, R¹ is

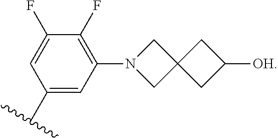 [SMILES: *c1cc(F)c(F)c(N2CC3(CC(O)C3)C2)c1]

In certain embodiments, R¹ is

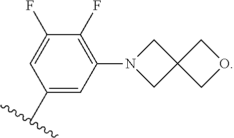 [SMILES: *c1cc(F)c(F)c(N2CC3(COC3)C2)c1]

In certain embodiments, R¹ is

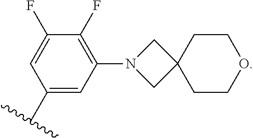 [SMILES: *c1cc(F)c(F)c(N2CC3(CCOCC3)C2)c1]

In certain embodiments, R¹ is

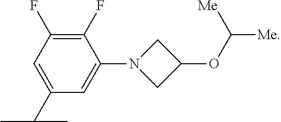 [SMILES: CC(C)OC1CN(c2cc(C(C)C)cc(F)c2F)C1]

In certain embodiments, R¹ is

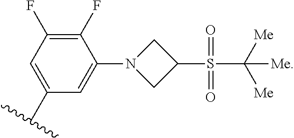 [SMILES: *c1cc(F)c(F)c(N2CC(S(=O)(=O)C(C)(C)C)C2)c1]

In certain embodiments, R¹ is

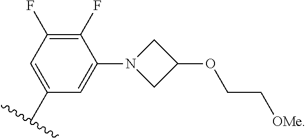 [SMILES: *c1cc(F)c(F)c(N2CC(OCCOC)C2)c1]

In certain embodiments, R¹ is

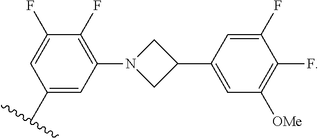 [SMILES: *c1cc(F)c(F)c(N2CC(c3cc(F)c(F)c(OC)c3)C2)c1]

In certain embodiments, R¹ is

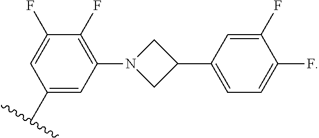 [SMILES: *c1cc(F)c(F)c(N2CC(c3ccc(F)c(F)c3)C2)c1]

In certain embodiments, R¹ is

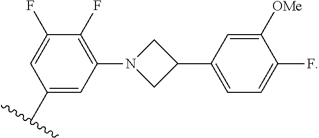 [SMILES: *c1cc(F)c(F)c(N2CC(c3ccc(F)c(OC)c3)C2)c1]

In certain embodiments, R¹ is

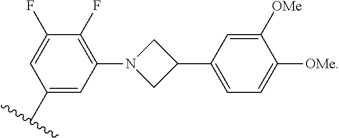 [SMILES: *c1cc(F)c(F)c(N2CC(c3ccc(OC)c(OC)c3)C2)c1]

In certain embodiments, R¹ is

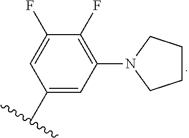 [SMILES: *c1cc(F)c(F)c(N2CCCC2)c1]

In certain embodiments, R¹ is

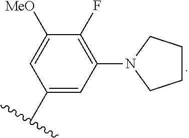 [SMILES: *c1cc(OC)c(F)c(N2CCCC2)c1]

In certain embodiments, R¹ is

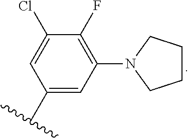 [SMILES: *c1cc(Cl)c(F)c(N2CCCC2)c1]

In certain embodiments, R¹ is

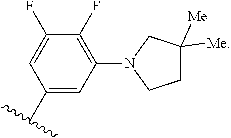 [SMILES: *c1cc(F)c(F)c(N2CCC(C)(C)C2)c1]

In certain embodiments, R¹ is

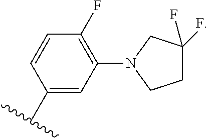 [SMILES: *c1ccc(F)c(N2CCC(F)(F)C2)c1]

In certain embodiments, R¹ is

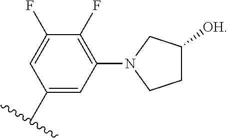 [SMILES: *c1cc(F)c(F)c(N2CC[C@@H](O)C2)c1]

In certain embodiments, R¹ is

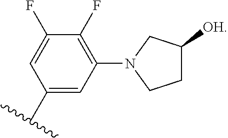 [SMILES: *c1cc(F)c(F)c(N2CC[C@H](O)C2)c1]

In certain embodiments, R¹ is

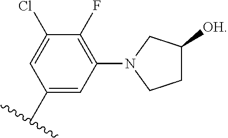 [SMILES: *c1cc(Cl)c(F)c(N2CC[C@H](O)C2)c1]

In certain embodiments, R¹ is

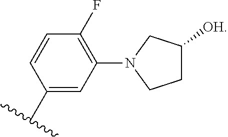 [SMILES: *c1ccc(F)c(N2CC[C@@H](O)C2)c1]

In certain embodiments, R¹ is

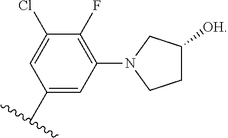 [SMILES: *c1cc(Cl)c(F)c(N2CC[C@@H](O)C2)c1]

In certain embodiments, R¹ is

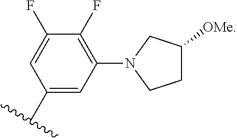 [SMILES: *c1cc(F)c(F)c(N2CC[C@@H](OC)C2)c1]

In certain embodiments, R¹ is

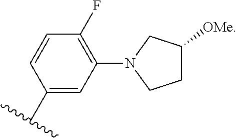 [SMILES: *c1ccc(F)c(N2CC[C@@H](OC)C2)c1]

In certain embodiments, R¹ is

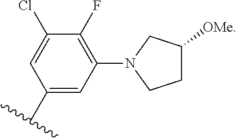 [SMILES: *c1cc(Cl)c(F)c(N2CC[C@@H](OC)C2)c1]

In certain embodiments, R¹ is

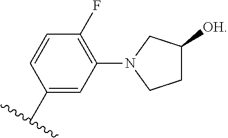 [SMILES: *c1ccc(F)c(N2CC[C@H](O)C2)c1]

In certain embodiments, R¹ is

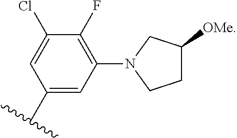 [SMILES: *c1cc(Cl)c(F)c(N2CC[C@H](OC)C2)c1]

In certain embodiments, R¹ is

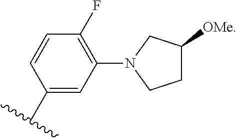 [SMILES: *c1ccc(F)c(N2CC[C@H](OC)C2)c1]

In certain embodiments, R¹ is

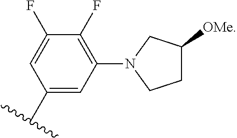 [SMILES: *c1cc(F)c(F)c(N2CC[C@H](OC)C2)c1]

In certain embodiments, R¹ is

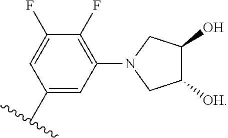 [SMILES: *c1cc(F)c(F)c(N2C[C@@H](O)[C@H](O)C2)c1]

In certain embodiments, R¹ is

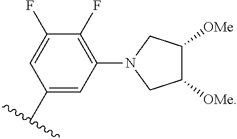 [SMILES: *c1cc(F)c(F)c(N2C[C@H](OC)[C@H](OC)C2)c1]

In certain embodiments, R¹ is

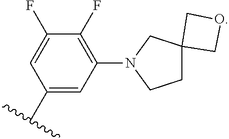 [SMILES: *c1cc(F)c(F)c(N2CCC3(COC3)C2)c1]

In certain embodiments, R¹ is

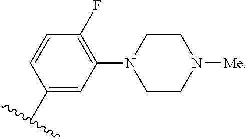 [SMILES: *c1ccc(F)c(N2CCN(C)CC2)c1]

In certain embodiments, R¹ is

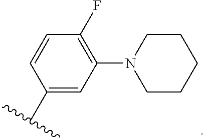 [SMILES: *c1ccc(F)c(N2CCCCC2)c1]

In certain embodiments, R¹ is

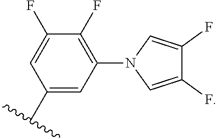 [SMILES: *c1cc(F)c(F)c(-n2cc(F)c(F)c2)c1]

In certain embodiments, R¹ is

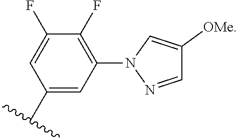 [SMILES: *c1cc(F)c(F)c(-n2cc(OC)cn2)c1]

In certain embodiments, R¹ is

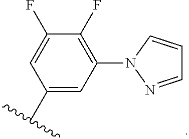 [SMILES: *c1cc(F)c(F)c(-n2cccn2)c1]

In certain embodiments, R¹ is

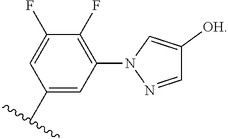 [SMILES: *c1cc(F)c(F)c(-n2cc(O)cn2)c1]

In certain embodiments, R¹ is

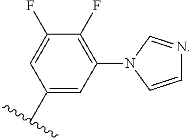 [SMILES: *c1cc(F)c(F)c(-n2ccnc2)c1]

In certain embodiments, R¹ is

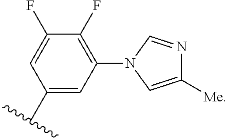 [SMILES: *c1cc(F)c(F)c(-n2cnc(C)c2)c1]

In certain embodiments, R¹ is

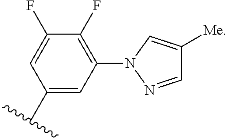 [SMILES: *c1cc(F)c(F)c(-n2cc(C)cn2)c1]

In certain embodiments, R¹ is

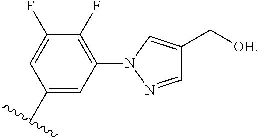 [SMILES: *c1cc(F)c(F)c(-n2cc(CO)cn2)c1]

In certain embodiments, R¹ is

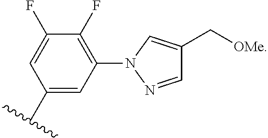 [SMILES: *c1cc(F)c(F)c(-n2cc(COC)cn2)c1]

In certain embodiments, R¹ is

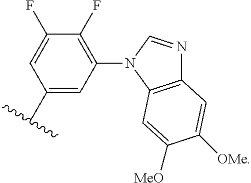 [SMILES: *c1cc(F)c(F)c(-n2cnc3cc(OC)c(OC)cc32)c1]

In certain embodiments, R¹ is

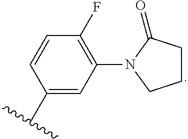 [SMILES: *c1ccc(F)c(N2CCCC2=O)c1]

In certain embodiments, R¹ is

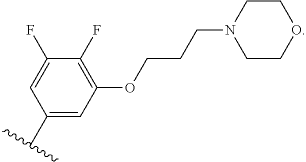 [SMILES: *c1cc(F)c(F)c(OCCCN2CCOCC2)c1]

In certain embodiments, R¹ is

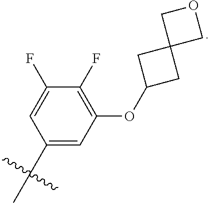 [SMILES: *c1cc(F)c(F)c(OC2CC3(COC3)C2)c1]

In certain embodiments, R¹ is

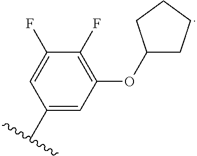 [SMILES: *c1cc(F)c(F)c(OC2CCCC2)c1]

In certain embodiments, R¹ is

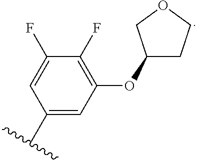 [SMILES: *c1cc(F)c(F)c(O[C@@H]2CCOC2)c1]

In certain embodiments, R¹ is

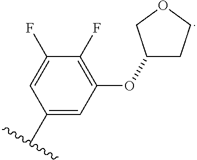

In certain embodiments, R¹ is

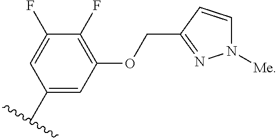 [SMILES: *c1cc(F)c(F)c(OCc2ccn(C)n2)c1]

In certain embodiments, R¹ is

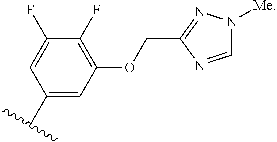 [SMILES: *c1cc(F)c(F)c(OCc2ncn(C)n2)c1]

In certain embodiments, R¹ is

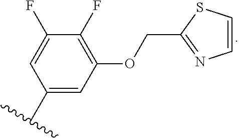 [SMILES: *c1cc(F)c(F)c(OCc2nccs2)c1]

In certain embodiments, R¹ is

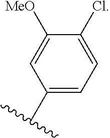 [SMILES: *c1ccc(Cl)c(OC)c1]

In certain embodiments, R¹ is

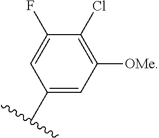 [SMILES: *c1cc(F)c(Cl)c(OC)c1]

In certain embodiments, R¹ is

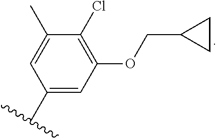 [SMILES: *c1cc(C)c(Cl)c(OCC2CC2)c1]

In certain embodiments, R¹ is

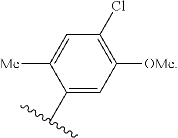 [SMILES: *c1cc(OC)c(Cl)cc1C]

In certain embodiments, R¹ is

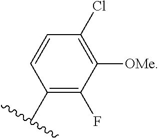 [SMILES: *c1ccc(Cl)c(OC)c1F]

In certain embodiments, R¹ is

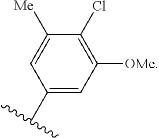 [SMILES: *c1cc(C)c(Cl)c(OC)c1]

In certain embodiments, R¹ is

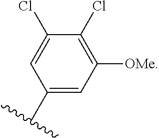 [SMILES: *c1cc(Cl)c(Cl)c(OC)c1]

In certain embodiments, R¹ is

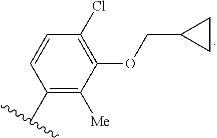 [SMILES: *c1ccc(Cl)c(OCC2CC2)c1C]

In certain embodiments, R¹ is

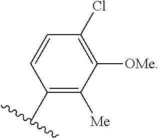 [SMILES: *c1ccc(Cl)c(OC)c1C]

In certain embodiments, R¹ is

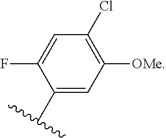 [SMILES: *c1cc(OC)c(Cl)cc1F]

In certain embodiments, R¹ is

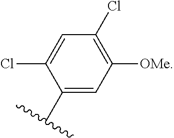 [SMILES: *c1cc(OC)c(Cl)cc1Cl]

In certain embodiments, R¹ is

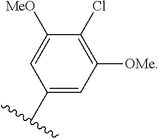 [SMILES: *c1cc(OC)c(Cl)c(OC)c1]

In certain embodiments, R¹ is

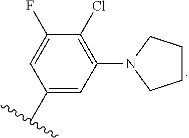 [SMILES: *c1cc(F)c(Cl)c(N2CCCC2)c1]

In certain embodiments, R¹ is

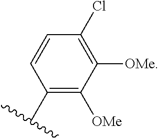 [SMILES: *c1ccc(Cl)c(OC)c1OC]

In certain embodiments, R¹ is

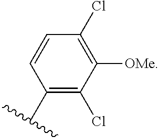 [SMILES: *c1ccc(Cl)c(OC)c1Cl]

In certain embodiments, R¹ is

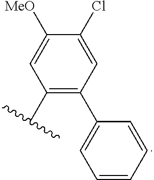 [SMILES: *c1cc(OC)c(Cl)cc1-c1ccccc1]

In certain embodiments, R¹ is

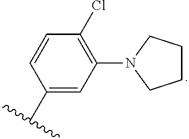 [SMILES: *c1ccc(Cl)c(N2CCCC2)c1]

In certain embodiments, R¹ is

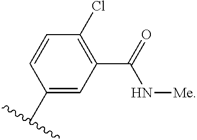 [SMILES: *c1ccc(Cl)c(C(=O)NC)c1]

In certain embodiments, R¹ is

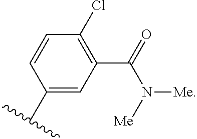 [SMILES: *c1ccc(Cl)c(C(=O)N(C)C)c1]

In certain embodiments, R¹ is

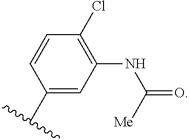 [SMILES: *c1ccc(Cl)c(NC(C)=O)c1]

In certain embodiments, R¹ is

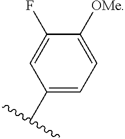 [SMILES: *c1ccc(OC)c(F)c1]

In certain embodiments, R¹ is

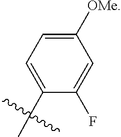 [SMILES: *c1ccc(OC)cc1F]

In certain embodiments, R¹ is

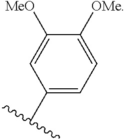 [SMILES: *c1ccc(OC)c(OC)c1]

In certain embodiments, R¹ is

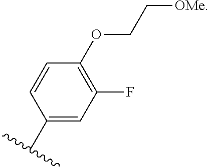 [SMILES: *c1ccc(OCCOC)c(F)c1]

In certain embodiments, R¹ is

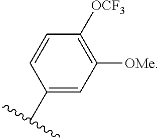 [SMILES: *c1ccc(OC(F)(F)F)c(OC)c1]

In certain embodiments, R¹ is

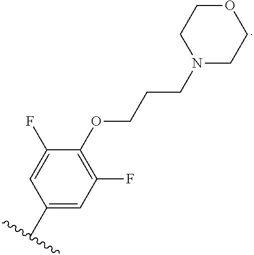 [SMILES: *c1cc(F)c(OCCCN2CCOCC2)c(F)c1]

In certain embodiments, R¹ is

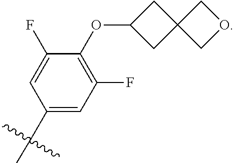 [SMILES: *c1cc(F)c(OC2CC3(COC3)C2)c(F)c1]

In certain embodiments, R¹ is

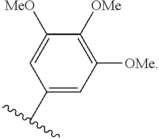 [SMILES: *c1cc(OC)c(OC)c(OC)c1]

In certain embodiments, R¹ is

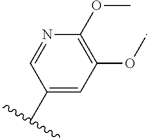

In certain embodiments, R¹ is

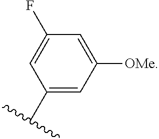 [SMILES: *c1cc(F)cc(OC)c1]

In certain embodiments, R¹ is

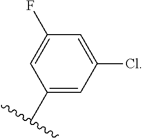 [SMILES: *c1cc(F)cc(Cl)c1]

In certain embodiments, R¹ is

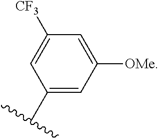 [SMILES: *c1cc(OC)cc(C(F)(F)F)c1]

In certain embodiments, R¹ is

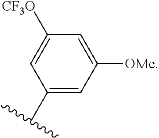

In certain embodiments, R¹ is

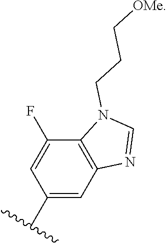 [SMILES: *c1cc(F)c2c(c1)ncn2CCCOC]

In certain embodiments, R¹ is

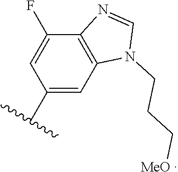 [SMILES: *c1cc(F)c2ncn(CCCOC)c2c1]

In certain embodiments, R¹ is

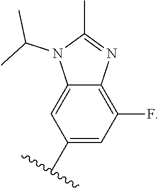 [SMILES: *c1cc(F)c2nc(C)n(C(C)C)c2c1]

In certain embodiments, R¹ is

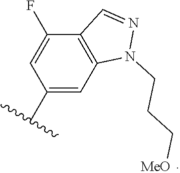 [SMILES: *c1cc(F)c2cnn(CCCOC)c2c1]

In certain embodiments, R¹ is

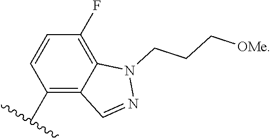 [SMILES: *c1ccc(F)c2c1cnn2CCCOC]

In certain embodiments, R¹ is

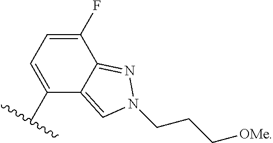 [SMILES: *c1ccc(F)c2nn(CCCOC)cc12]

In certain embodiments, R¹ is

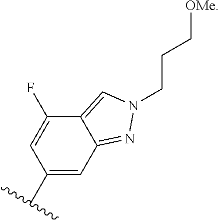 [SMILES: *c1cc(F)c2cn(CCCOC)nc2c1]

In certain embodiments, R¹ is

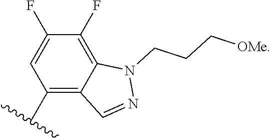 [SMILES: *c1cc(F)c(F)c2c1cnn2CCCOC]

In certain embodiments, R¹ is

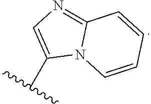 [SMILES: *c1cnc2ccccn12]

In certain embodiments, R¹ is

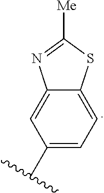 [SMILES: *c1ccc2sc(C)nc2c1]

In certain embodiments, R¹ is

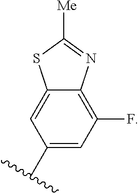 [SMILES: *c1cc(F)c2nc(C)sc2c1]

In certain embodiments, R¹ is

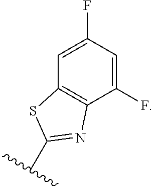 [SMILES: *c1nc2c(F)cc(F)cc2s1]

In certain embodiments, R¹ is

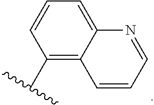 [SMILES: *c1cccc2ncccc12]

In certain embodiments, R¹ is

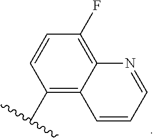 [SMILES: *c1ccc(F)c2ncccc12]

In certain embodiments, R¹ is

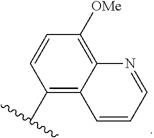 [SMILES: *c1ccc(OC)c2ncccc12]

In certain embodiments, R¹ is

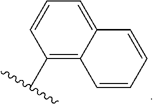 [SMILES: *c1cccc2ccccc12]

In certain embodiments, R¹ is

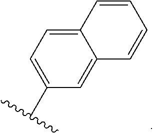 [SMILES: *c1ccc2ccccc2c1]

In certain embodiments, R¹ is

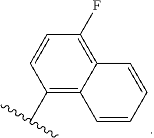 [SMILES: *c1ccc(F)c2ccccc12]

In certain embodiments, R¹ is

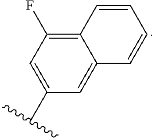 [SMILES: *c1cc(F)c2ccccc2c1]

In certain embodiments, each occurrence of R^{2a}, R^{2b}, R^{2c}, R^{2d}, and R^{2e} is independently selected from the group consisting of H, F, Cl, Br, I, methyl, ethyl, isopropyl, cyclopropyl, cyclobutyl, cyclopentyl, cyclohexyl, methoxy, ethoxy, isopropoxy, phenyl, optionally substituted benzo[d]thiazolyl, azetidinyl, pyrrolidinyl, piperidinyl, and piperazinyl. In certain embodiments, each occurrence of R^{2a}, R^{2b}, R^{2c}, R^{2d}, and R^{2e} is independently selected from the group consisting of H, methyl, ethyl, isopropyl, cyclopropyl, cyclobutyl, cyclopentyl, cyclohexyl, methoxy, ethoxy, isopropoxy, phenyl, optionally substituted benzo[d]thiazolyl, azetidinyl, pyrrolidinyl, piperidinyl, and piperazinyl.

In certain embodiments, in (Ib), (Id), (If), (Id1), (Id2), (If1), and/or (If2), R^{2a}, and/or R^{2b} if X² is CR^{2b}, is/are independently selected from the group consisting of H, methyl, ethyl, isopropyl, cyclopropyl, cyclobutyl, cyclopentyl, cyclohexyl, methoxy, ethoxy, isopropoxy, phenyl, optionally substituted benzo[d]thiazolyl, azetidinyl, pyrrolidinyl, piperidinyl, and piperazinyl.

In certain embodiments, in (Ib 1), (Ib2), (Ib3), (Ib4), (Ib5), and/or (Ib6), R^{2a} and/or R^{2b} is/are independently selected from the group consisting of H, methyl, ethyl, isopropyl, cyclopropyl, cyclobutyl, cyclopentyl, cyclohexyl, methoxy, ethoxy, isopropoxy, phenyl, optionally substituted benzo[d]thiazolyl, azetidinyl, pyrrolidinyl, piperidinyl, and piperazinyl.

In certain embodiments, in (IIb), (IIe), (IIh), (IIe1), (IIe2), (IIh1), (IIh2), (IIb1), (IIb2), and/or (IIb3), R^{2b} and/or R^{2c} is/are independently selected from the group consisting of H, methyl, ethyl, isopropyl, cyclopropyl, cyclobutyl, cyclopentyl, cyclohexyl, methoxy, ethoxy, isopropoxy, phenyl, optionally substituted benzo[d]thiazolyl, azetidinyl, pyrrolidinyl, piperidinyl, and piperazinyl.

In certain embodiments, in (IIIb), (IIId), (IIIf), (IIId1), (IIId2), (IIIf2), (IIIb1), (IIIb2), and/or (IIIb3), R^{2a} is independently selected from the group consisting of H, methyl, ethyl, isopropyl, cyclopropyl, cyclobutyl, cyclopentyl, cyclohexyl, methoxy, ethoxy, isopropoxy, phenyl, optionally substituted benzo[d]thiazolyl, azetidinyl, pyrrolidinyl, piperidinyl, and piperazinyl.

In certain embodiments, each occurrence of R^{3a}, R^{3b}, R^{3c}, and R^{3d} is independently selected from the group consisting of H, halogen, cyano, nitro, optionally substituted C₁-C₆ alkyl, C₁-C₆ haloalkyl, optionally substituted C₃-C₈ cycloalkyl, C₁-C₆ haloalkoxy, C₁-C₆ hydroxyalkyl, and -OR″″, wherein each occurrence of R″″ is independently selected from the group consisting of H, optionally substituted C₁-C₆ alkyl, and optionally substituted C₃-C₈ cycloalkyl. In certain embodiments, each occurrence of R^{3a}, R^{3b}, R^{3c}, and R^{3d} is independently selected from the group consisting of H, halogen, cyano, nitro, and optionally substituted C₁-C₆ alkyl. In certain embodiments, each occurrence of R^{3a}, R^{3b}, R^{3c}, and R^{3d} is independently selected from the group consisting of H, halogen, and optionally substituted C₁-C₆ alkyl. In certain embodiments, each occurrence of R^{3a}, R^{3b}, R^{3c}, and R^{3d} is H.

In certain embodiments, each occurrence of R^{3b} is independently H, methyl, ethyl, propyl, cyclopropyl, isopropyl, methoxy, ethoxy, propoxy, cyclopropoxy, isopropoxy, fluoro, chloro, bromo, or iodo.

In certain embodiments, R^{4a} is H. In certain embodiments, R^{4a} is optionally substituted C₁-C₆ alkyl. In certain embodiments, R^{4a} is optionally substituted C₃-C₈ cycloalkyl. In certain embodiments, R^{4a} is F, Cl, Br, or I. In certain embodiments, R^{4a} is —OR^{4c}. In certain embodiments, R^{4a} is —C(═O)OR^{4c}. In certain embodiments, R^{4c} is H. In certain embodiments, R^{4c} is optionally substituted C₁-C₆ alkyl. In certain embodiments, R^{4c} is optionally substituted C₃-C₈ cycloalkyl.

In certain embodiments, R^{4b} is H. In certain embodiments, R^{4b} is optionally substituted C₁-C₆ alkyl. In certain embodiments, R^{4b} is optionally substituted C₃-C₈ cycloalkyl. In certain embodiments, R^{4b} is F, Cl, Br, or I. In certain embodiments, R^{4b} is —OR^{4d}. In certain embodiments, R^{4b} is —C(═O)OR^{4d}. In certain embodiments, R^{4d} is H. In certain embodiments, R^{4d} is optionally substituted C₁-C₆ alkyl. In certain embodiments, R^{4d} is optionally substituted C₃-C₈ cycloalkyl.

In certain embodiments, each occurrence of alkyl, alkylenyl (alkylene), cycloalkyl, heterocyclyl, or carbocyclyl is independently optionally substituted with at least one substituent selected from the group consisting of C₁-C₆ alkyl, halogen, —OR″′, phenyl (thus yielding, in non-limiting examples, optionally substituted phenyl-(C₁-C₃ alkyl), such as, but not limited to, benzyl or substituted benzyl), —S(O)₂R″′, and —N(R″′)(R″′), wherein each occurrence of R″′ is independently H, optionally substituted C₁-C₆ alkyl, or optionally substituted C₃-C₈ cycloalkyl.

In certain embodiments, each occurrence of aryl or heteroaryl is independently optionally substituted with at least one substituent selected from the group consisting of C₁-C₆ alkyl, C₁-C₆ haloalkyl, C₁-C₆ haloalkoxy, halogen, —CN, —OR″″, —N(R″″)(R″″), —NO₂, S(O)₂R″″, —S(═O)₂N(R″″)(R″″), acyl, and C₁-C₆ alkoxycarbonyl, wherein each occurrence of R″″ is independently H, optionally substituted C₁-C₆ alkyl, or optionally substituted C₃-C₈ cycloalkyl.

In certain embodiments, each occurrence of aryl or heteroaryl is independently optionally substituted with at least one substituent selected from the group consisting of C₁-C₆ alkyl, C₁-C₆ haloalkyl, C₁-C₆ haloalkoxy, halogen, —CN, —OR″″, —N(R″″)(R″″), and C₁-C₆ alkoxycarbonyl, wherein each occurrence of R″″ is independently H, optionally substituted C₁-C₆ alkyl, or optionally substituted C₃-C₈ cycloalkyl.

In certain embodiments, each occurrence of the heteroaryl is independently selected from the group consisting of quinolinyl, imidazo[1,2-a]pyridyl, 1H-indazolyl, pyridyl, pyrimidyl, pyrazinyl, imidazolyl, pyrrolyl, thiazolyl, pyrazolyl, isoxazolyl, oxadiazolyl (including 1,2,3-, 1,2,4-, 1,2,5-, and 1,3,4-oxadiazole), tetrazolyl, triazolyl (such as, but not limited to, 1,2,4-triazolyl or 1,2,3-triazolyl), benzo[d]thiazolyl, and benzo[d]imidazolyl.

In certain embodiments, each occurrence of the heterocyclyl group is independently selected from the group consisting of tetrahydrofuranyl, tetrahydropyranyl, piperidinyl, piperazinyl, pyrrolidinyl, morpholinyl, thiomorpholinyl, 1-oxido-thiomorpholinyl, 1,1-dioxido-thiomorpholinyl, oxetanyl, oxazolidinyl, azetidinyl, 7-oxa-2-azaspiro[3.5]nonyl, 2-oxa-6-azaspiro[3.4]octyl, 2-oxa-6-azaspiro[3.3]heptyl, 2-azaspiro[3.3]hept-2-yl, 2-oxaspiro[3.3]hept-6-yl; and the corresponding oxo analogues (where a methylene ring group is replaced with a carbonyl) thereof, such as but not limited to pyrrolidinonyl, piperidinonyl, piperazinonyl, and/or morpholinonyl.

In certain embodiments, the compound of formula (I) is

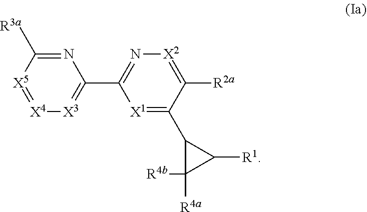

In certain embodiments, the compound of formula (I) is

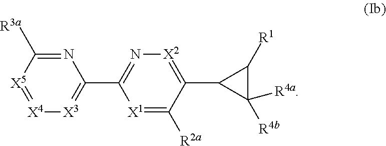

In certain embodiments, the compound of formula (II) is

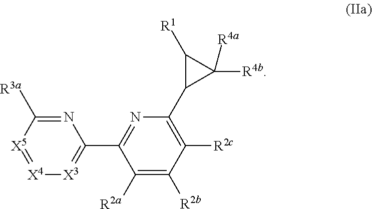

In certain embodiments, the compound of formula (II)

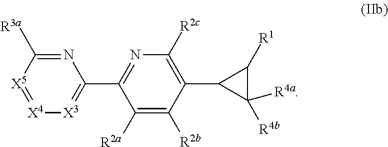

is In certain embodiments, the compound of formula (II) is

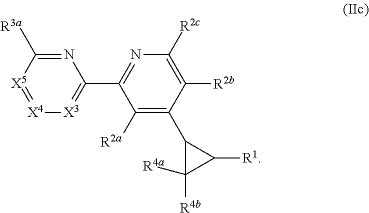

In certain embodiments, the compound of formula (III) is

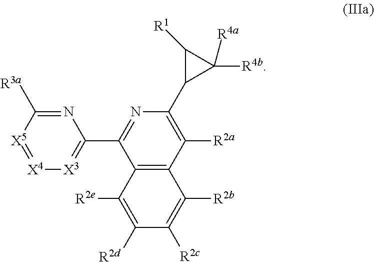

In certain embodiments, the compound of formula (III) is

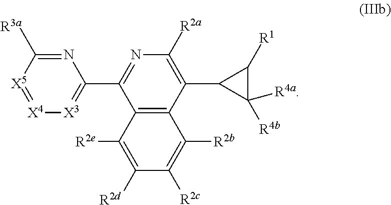

In certain embodiments, the compound of formula (I) is

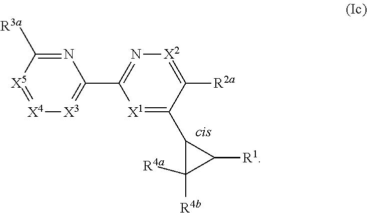

In certain embodiments, the compound of formula (I) is

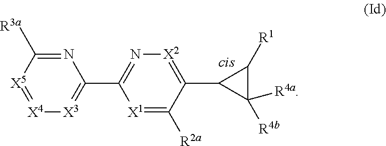

In certain embodiments, the compound of formula (II) is

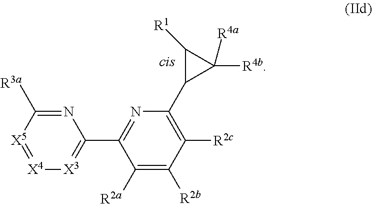

In certain embodiments, the compound of formula (II) is

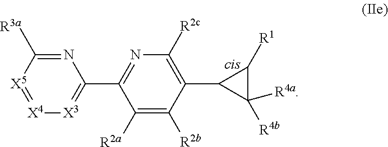

is In certain embodiments, the compound of formula (II) is

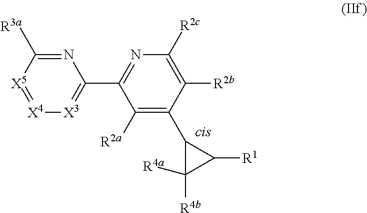

In certain embodiments, the compound of formula (III) is

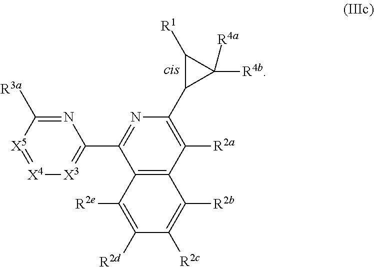

In certain embodiments, the compound of formula (III) is

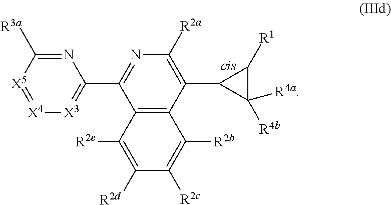

In certain embodiments, the compound of formula (I) is

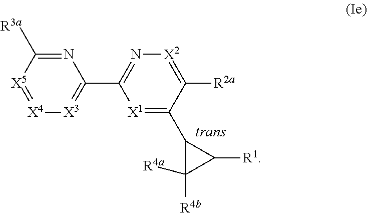

In certain embodiments, the compound of formula (I) is

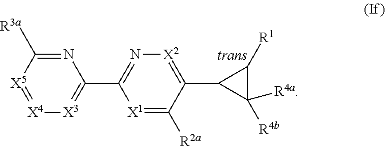

In certain embodiments, the compound of formula (II) is

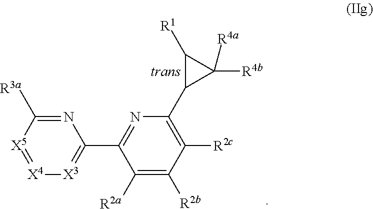

In certain embodiments, the compound of formula (II) is

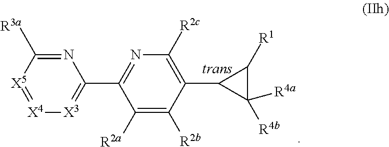

In certain embodiments, the compound of formula (II) is

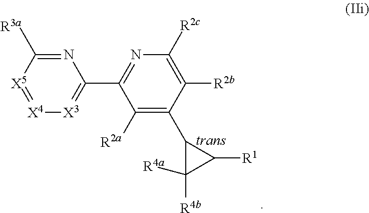

In certain embodiments, the compound of formula (III) is

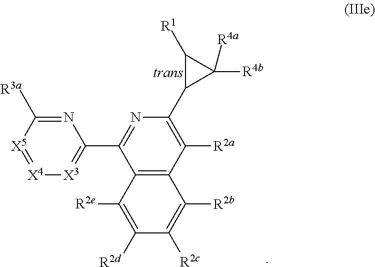

In certain embodiments, the compound of formula (III) is

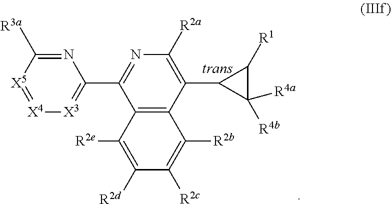

In certain embodiments, the compound of formula (I) is

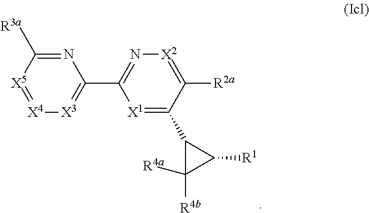

In certain embodiments, the compound of formula (I) is

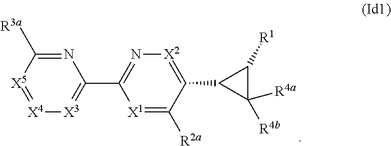

In certain embodiments, the compound of formula (I) is

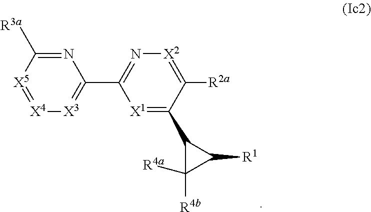

In certain embodiments, the compound of formula (I) is

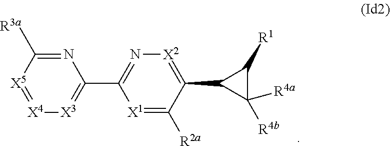

In certain embodiments, the compound of formula (II) is

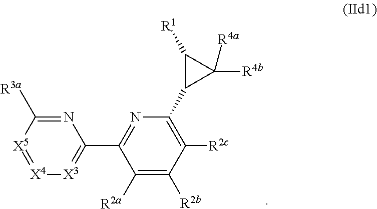

In certain embodiments, the compound of formula (II) is

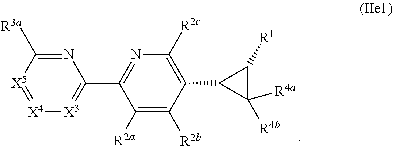

In certain embodiments, the compound of formula (II) is

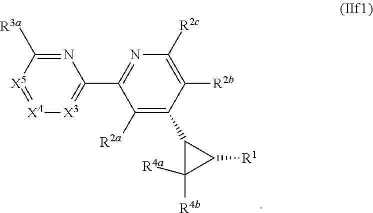

In certain embodiments, the compound of formula (II) is

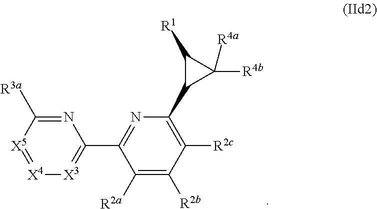

In certain embodiments, the compound of formula (II) is

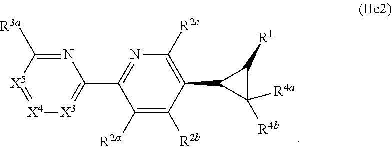

In certain embodiments, the compound of formula (II) is

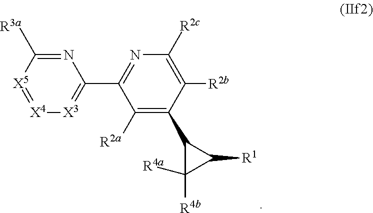

In certain embodiments, the compound of formula (III) is

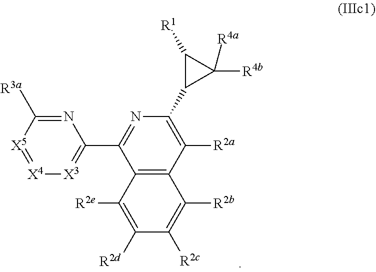

In certain embodiments, the compound of formula (III) is

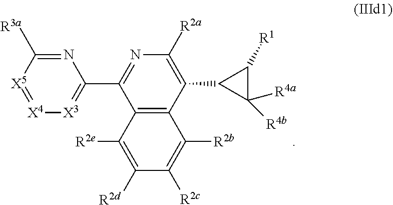

In certain embodiments, the compound of formula (III) is

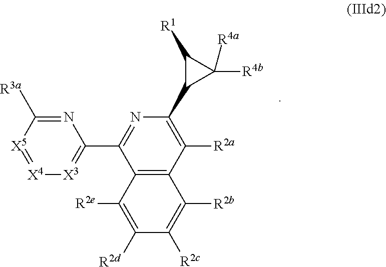

In certain embodiments, the compound of formula (III) is

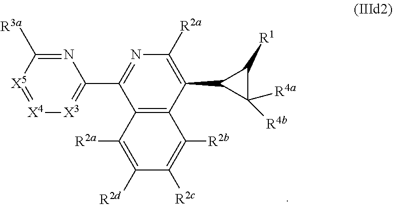

In certain embodiments, the compound of formula (I) is

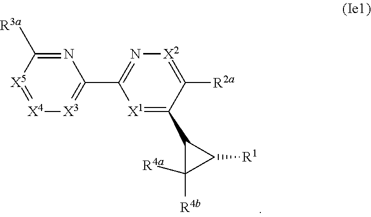

In certain embodiments, the compound of formula (I) is

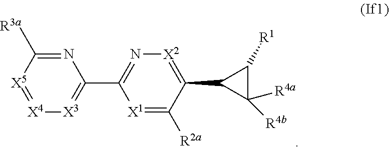

In certain embodiments, the compound of formula (I) is

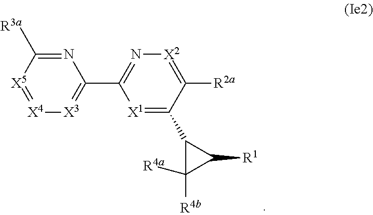

In certain embodiments, the compound of formula (I) is

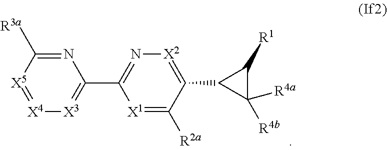

In certain embodiments, the compound of formula (II) is

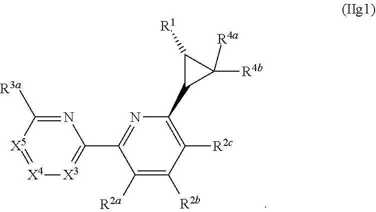

In certain embodiments, the compound of formula (II) is

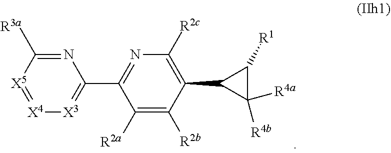

In certain embodiments, the compound of formula (II) is

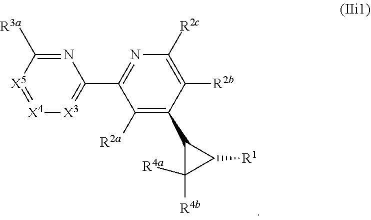

In certain embodiments, the compound of formula (II) is

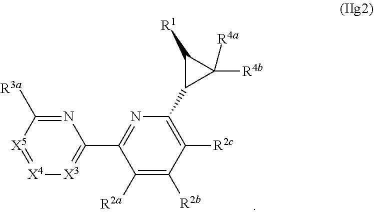

In certain embodiments, the compound of formula (II) is

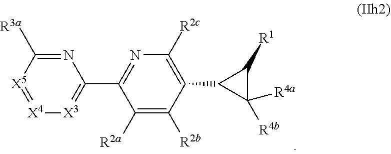

In certain embodiments, the compound of formula (II) is

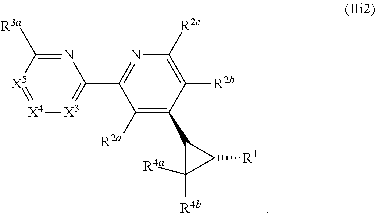

In certain embodiments, the compound of formula (III) is

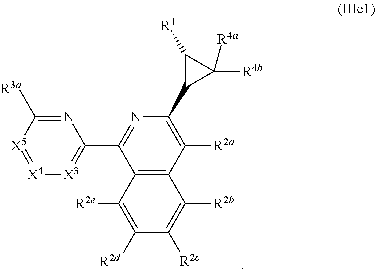

In certain embodiments, the compound of formula (III) is

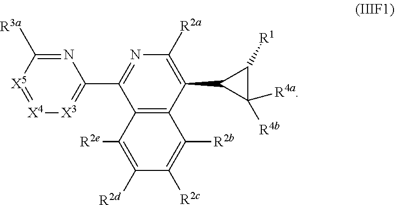

In certain embodiments, the compound of formula (III) is

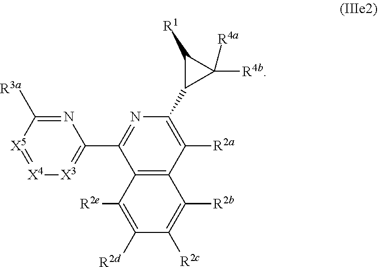

In certain embodiments, the compound of formula (III) is

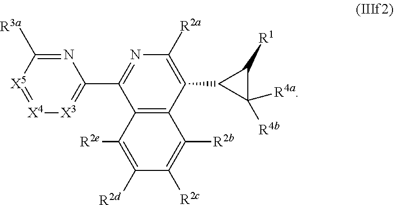

In certain embodiments, the compound of formula (I) is

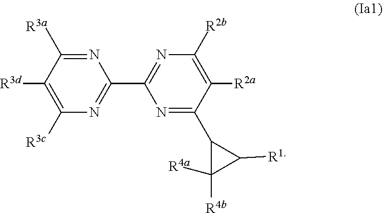

In certain embodiments, the compound of formula (I) is

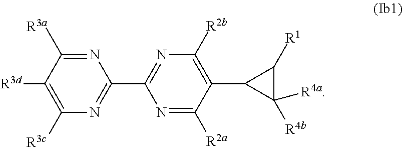

In certain embodiments, the compound of formula (I) is

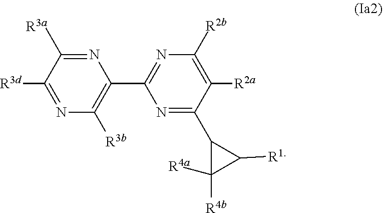

In certain embodiments, the compound of formula (I) is

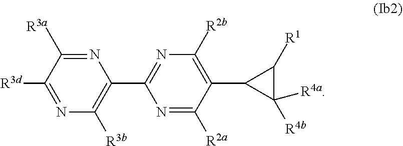

In certain embodiments, the compound of formula (I) is

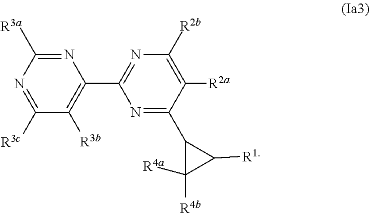

In certain embodiments, the compound of formula (I) is

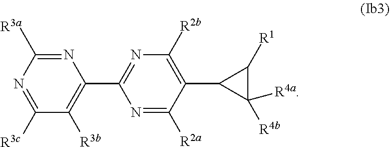

In certain embodiments, the compound of formula (I) is

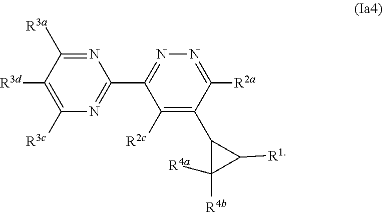

In certain embodiments, the compound of formula (I) is

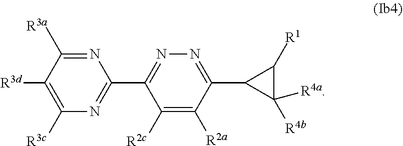

In certain embodiments, the compound of formula (I) is

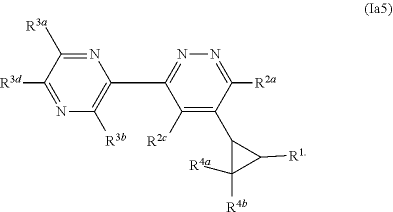

In certain embodiments, the compound of formula (I) is

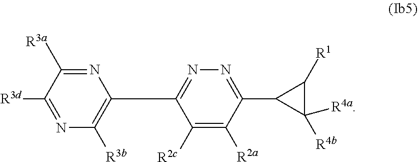

In certain embodiments, the compound of formula (I) is

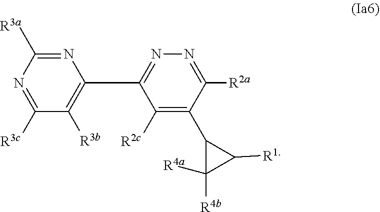

In certain embodiments, the compound of formula (I) is

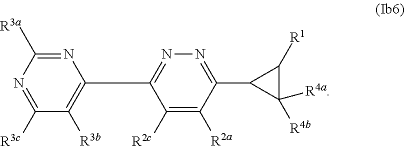

In certain embodiments, the compound of formula (II) is

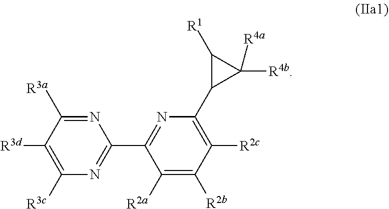

In certain embodiments, the compound of formula (II) is

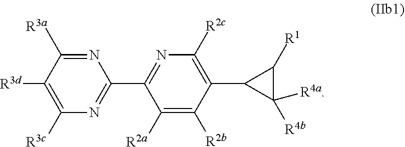

In certain embodiments, the compound of formula (II) is

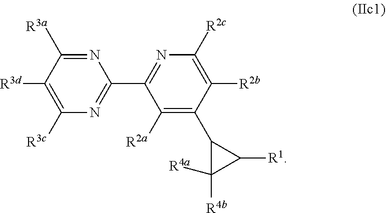

In certain embodiments, the compound of formula (II) is

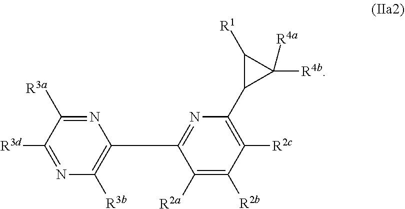

In certain embodiments, the compound of formula (II) is

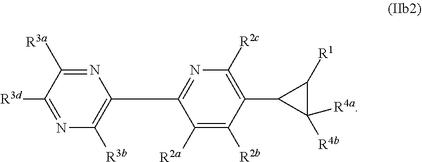

In certain embodiments, the compound of formula (II) is

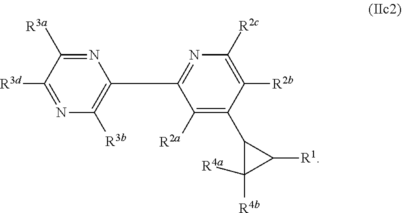

In certain embodiments, the compound of formula (II) is

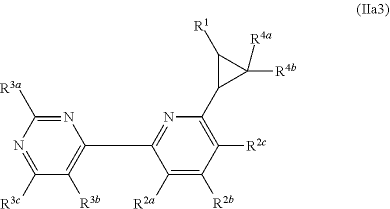

In certain embodiments, the compound of formula (II) is

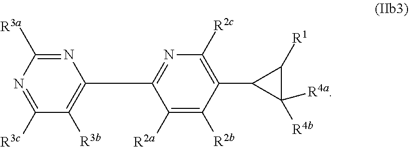

In certain embodiments, the compound of formula (II) is

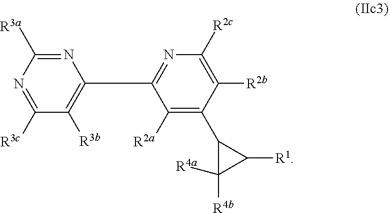

In certain embodiments, the compound of formula (III) is

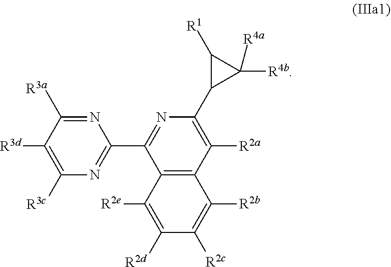

In certain embodiments, the compound of formula (III) is

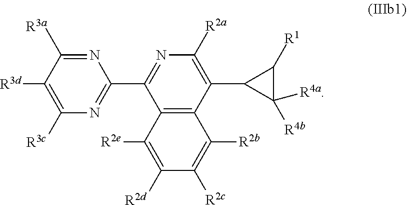

In certain embodiments, the compound of formula (III) is

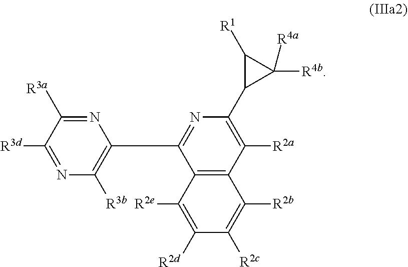

In certain embodiments, the compound of formula (III) is

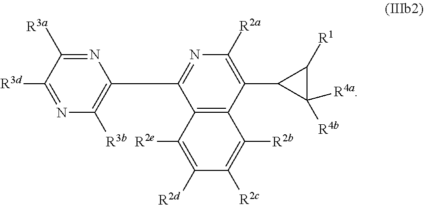

In certain embodiments, the compound of formula (III) is

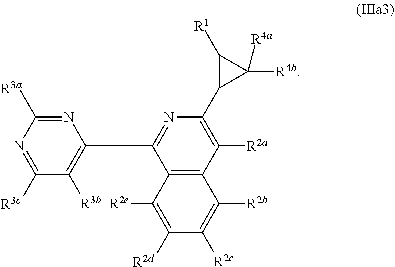

In certain embodiments, the compound of formula (III) is

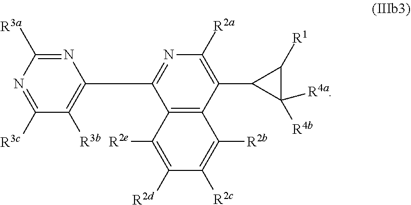

In certain embodiments, the compound of the disclosure is any compound disclosed herein, or a salt, solvate, isotopically labeled (such as for example at least partially deuterated), stereoisomer, any mixture of stereoisomers, tautomer, and/or any mixture of tautomers thereof.

In certain embodiments, the compound is at least one selected from Table 1, or a salt, solvate, isotopically labeled, stereoisomer, any mixture of stereoisomers, tautomer, and/or any mixture of tautomers thereof.

In certain embodiments, the compound, or a salt, solvate, isotopically labeled, stereoisomer, any mixture of stereoisomers, tautomer, and/or any mixture of tautomers thereof, is one of the following:
5-(2-(2-fluoro-4-methoxyphenyl)cyclopropyl)-2,2′-bipyrimidine;
4-(2-(3,4-difluorophenyl)cyclopropyl)-2,2′-bipyrimidine;
5-(2-(4-fluoro-2-methoxyphenyl)cyclopropyl)-2,2′-bipyrimidine;
5-(2-(4-fluoro-3-methoxyphenyl)cyclopropyl)-2,2′-bipyrimidine;
5-(2-(3-fluoro-4-methoxyphenyl)cyclopropyl)-2,2′-bipyrimidine;
5-(2-(3,4-difluorophenyl)cyclopropyl)-2,2′-bipyrimidine;
5-(2-(4-fluoro-3-methoxyphenyl)cyclopropyl)-4-methyl-2,2′-bipyrimidine;
5-(2-(4-fluoro-3-(pyrrolidin-1-yl)phenyl)cyclopropyl)-2,2′-bipyrimidine;
5-(2-(4-fluoro-3-(2-methoxyethoxy)phenyl)cyclopropyl)-2,2′-bipyrimidine;
5-(2-(3-fluoro-4-(2-methoxyethoxy)phenyl)cyclopropyl)-2,2′-bipyrimidine;
5-(2-(3-(cyclopropylmethoxy)-4-fluorophenyl)cyclopropyl)-2,2′-bipyrimidine;
5-(2-(3-(2,2-difluoroethoxy)-4-fluorophenyl)cyclopropyl)-2,2′-bipyrimidine;
5-(2-(3-chloro-4-fluorophenyl)cyclopropyl)-2,2′-bipyrimidine;
5-(2-(3,4-dimethoxyphenyl)cyclopropyl)-2,2′-bipyrimidine;
5-(2-(4-chloro-3-methoxyphenyl)cyclopropyl)-2,2′-bipyrimidine;
5-(2-(2-ethyl-4-fluoro-5-methoxyphenyl)cyclopropyl)-2,2′-bipyrimidine;
5-(2-(4-fluoro-5-methoxy-2-propylphenyl)cyclopropyl)-2,2′-bipyrimidine;
5-(2-(4-fluoro-3-(trifluoromethoxy)phenyl)cyclopropyl)-2,2′-bipyrimidine;
5-(2-(4-fluoro-3-(4-(methylsulfonyl)piperazin-1-yl)phenyl)cyclopropyl)-2,2′-bipyrimidine;
5-(2-(3-(3,3-difluoropyrrolidin-1-yl)-4-fluorophenyl)cyclopropyl)-2,2′-bipyrimidine;
5-(2-(4-chloro-3-fluoro-5-methoxyphenyl)cyclopropyl)-2,2′-bipyrimidine;
5-(2-(4-fluoro-3-(3-methoxyazetidin-1-yl)phenyl)cyclopropyl)-2,2′-bipyrimidine;
5-((2-(4-chloro-3-(cyclopropylmethoxy)-5-methylphenyl)cyclopropyl)-2,2′-bipyrimidine;
5-(2-(4-chloro-5-methoxy-2-methylphenyl)cyclopropyl)-2,2′-bipyrimidine;
1-(5-(2-([2,2′-bipyrimidin]-5-yl)cyclopropyl)-2-fluorophenyl)azetidin-3-ol;
5-(2-(4-chloro-2-fluoro-3-methoxyphenyl)cyclopropyl)-2,2′-bipyrimidine;
5-(2-(4-chloro-3-methoxy-5-methylphenyl)cyclopropyl)-2,2′-bipyrimidine;
5-(2-(3,4-dichloro-5-methoxyphenyl)cyclopropyl)-2,2′-bipyrimidine;
5-(2-(4-chloro-3-(cyclopropylmethoxy)-2-methylphenyl)cyclopropyl)-2,2′-bipyrimidine;
5-(2-([2,2′-bipyrimidin]-5-yl)cyclopropyl)-2-fluoro-N,N-dimethylaniline;
(3S)-1-(5-(2-([2,2′-bipyrimidin]-5-yl)cyclopropyl)-2-fluorophenyl)pyrrolidin-3-ol;
5-(2-(4-fluoro-3-((S)-3-methoxypyrrolidin-1-yl)phenyl)cyclopropyl)-2,2′-bipyrimidine;
(3R)-1-(5-(2-([2,2′-bipyrimidin]-5-yl)cyclopropyl)-2-fluorophenyl)pyrrolidin-3-ol;
5-(2-(4-chloro-3-methoxy-2-methylphenyl)cyclopropyl)-2,2′-bipyrimidine;
5-(2-(4-fluoro-3-methoxyphenyl)cyclopropyl)-4-isopropyl-2,2′-bipyrimidine;
4-ethyl-5-(2-(4-fluoro-3-methoxyphenyl)cyclopropyl)-2,2′-bipyrimidine;
4-cyclohexyl-5-(2-(4-fluoro-3-methoxyphenyl)cyclopropyl)-2,2′-bipyrimidine;
5-(2-(4-fluoro-3-methoxyphenyl)cyclopropyl)-4-methoxy-2,2′-bipyrimidine;
5-(2-(3-(azetidin-1-yl)-4-fluorophenyl)cyclopropyl)-2,2′-bipyrimidine;
5-(2-(3,4-difluoro-5-methoxyphenyl)cyclopropyl)-2,2′-bipyrimidine;
5-(2-(4-chloro-2-fluoro-5-methoxyphenyl)cyclopropyl)-2,2′-bipyrimidine;
5-(2-(2,4-dichloro-5-methoxyphenyl)cyclopropyl)-2,2′-bipyrimidine;
5-(2-(4-chloro-3-fluoro-5-(pyrrolidin-1-yl)phenyl)cyclopropyl)-2,2′-bipyrimidine;
5-(2-(3,4-difluoro-5-(pyrrolidin-1-yl)phenyl)cyclopropyl)-2,2′-bipyrimidine;
5-(2-(4-fluoro-3-((R)-3-methoxypyrrolidin-1-yl)phenyl)cyclopropyl)-2,2′-bipyrimidine;
5-(2-(3,4-difluoro-5-((R)-3-methoxypyrrolidin-1-yl)phenyl)cyclopropyl)-2,2′-bipyrimidine;
5-(2-(4-chloro-3,5-dimethoxyphenyl)cyclopropyl)-2,2′-bipyrimidine;
5-(2-(4-fluoro-3-methoxy-5-(pyrrolidin-1-yl)phenyl)cyclopropyl)-2,2′-bipyrimidine;
5-(2-(3,4-difluoro-5-((S)-3-methoxypyrrolidin-1-yl)phenyl)cyclopropyl)-2,2′-bipyrimidine;
(3R)-1-(5-(2-([2,2′-bipyrimidin]-5-yl)cyclopropyl)-2,3-difluorophenyl)pyrrolidin-3-ol;
(3S)-1-(5-(2-([2,2′-bipyrimidin]-5-yl)cyclopropyl)-2,3-difluorophenyl)pyrrolidin-3-ol;
5-(2-(3-chloro-4-fluoro-5-(pyrrolidin-1-yl)phenyl)cyclopropyl)-2,2′-bipyrimidine;
5-(2-(3-chloro-4-fluoro-5-((R)-3-methoxypyrrolidin-1-yl)phenyl)cyclopropyl)-2,2′-bipyrimidine;
5-(2-(3-fluoro-5-methoxyphenyl)cyclopropyl)-2,2′-bipyrimidine;
5-(2-(4-chloro-2,3-dimethoxyphenyl)cyclopropyl)-2,2′-bipyrimidine;
5-(2-(3-chloro-4-fluoro-5-((S)-3-methoxypyrrolidin-1-yl)phenyl)cyclopropyl)-2,2′-bipyrimidine;
5-(2-(3-chloro-5-fluorophenyl)cyclopropyl)-2,2′-bipyrimidine;
(3S)-1-(5-(2-([2,2′-bipyrimidin]-5-yl)cyclopropyl)-3-chloro-2-fluorophenyl)pyrrolidin-3-ol;
5-(2-(3,4-difluoro-5-(3-methoxyazetidin-1-yl)phenyl)cyclopropyl)-2,2′-bipyrimidine;
2-(5-(2-(3,4-difluoro-5-methoxyphenyl)cyclopropyl)pyridin-2-yl)pyrimidine;
(3R)-1-(5-(2-([2,2′-bipyrimidin]-5-yl)cyclopropyl)-3-chloro-2-fluorophenyl)pyrrolidin-3-ol;
5-(2-(3-(cyclopropylmethoxy)-4,5-difluorophenyl)cyclopropyl)-2,2′-bipyrimidine;
5-(2-(3,4-difluoro-5-(3-methoxypropoxy)phenyl)cyclopropyl)-2,2′-bipyrimidine;
5-(2-(2,4-dichloro-3-methoxyphenyl)cyclopropyl)-2,2′-bipyrimidine;
5-(2-(3,4-difluoro-5-(2-methoxyethoxy)phenyl)cyclopropyl)-2,2′-bipyrimidine;
2-(5-(2-([2,2′-bipyrimidin]-5-yl)cyclopropyl)-2,3-difluorophenyl)-7-oxa-2-azaspiro[3.5]nonane;
5-(2-(3-(3,3-dimethylazetidin-1-yl)-4,5-difluorophenyl)cyclopropyl)-2,2′-bipyrimidine;
6-(5-(2-(4-fluoro-3-methoxyphenyl)cyclopropyl)-[2,2′-bipyrimidin]-4-yl)-2-methylbenzo[d]thiazole;
5-(2-(4-Fluoro-3-methoxyphenyl)cyclopropyl)-4-phenyl-2,2′-bipyrimidine;
5-(2-(4-fluoro-3-methoxyphenyl)cyclopropyl)-4-(piperidin-1-yl)-2,2′-bipyrimidine;
5-(2-(4-fluoro-3,5-dimethoxyphenyl)cyclopropyl)-2,2′-bipyrimidine;
5-(2-(3-chloro-4-fluoro-5-methoxyphenyl)cyclopropyl)-2,2′-bipyrimidine;
5-(2-(4-fluoro-3-methoxy-5-methylphenyl)cyclopropyl)-2,2′-bipyrimidine;
5-(2-(3-methoxy-4-(trifluoromethoxy)phenyl)cyclopropyl)-2,2′-bipyrimidine;
5-(2-(3-methoxy-4-(trifluoromethyl)phenyl)cyclopropyl)-2,2′-bipyrimidine;
5-(2-(5-chloro-4-fluoro-2-(3-methoxypropoxy)phenyl)cyclopropyl)-2,2′-bipyrimidine;
5-(2-(5-chloro-4-fluoro-2-(2-methoxyethoxy)phenyl)cyclopropyl)-2,2′-bipyrimidine;
5-(2-(5-chloro-4-methoxy-[1,1′-biphenyl]-2-yl)cyclopropyl)-2,2′-bipyrimidine;
5-(2-(3,4,5-trifluorophenyl)cyclopropyl)-2,2′-bipyrimidine;
5-(2-(3-methoxy-5-(trifluoromethyl)phenyl)cyclopropyl)-2,2′-bipyrimidine;
5-(2-(3-methoxy-5-(trifluoromethoxy)phenyl)cyclopropyl)-2,2′-bipyrimidine;
5-(2-(4-fluoro-3-methoxy-5-(trifluoromethyl)phenyl)cyclopropyl)-2,2′-bipyrimidine;
5-(2-(naphthalen-1-yl)cyclopropyl)-2,2′-bipyrimidine;
5-(2-(naphthalen-2-yl)cyclopropyl)-2,2′-bipyrimidine;
5-(2-(4-fluoronaphthalen-1-yl)cyclopropyl)-2,2′-bipyrimidine;
5-(2-(4-fluoronaphthalen-2-yl)cyclopropyl)-2,2′-bipyrimidine;
3-(2-([2,2′-bipyrimidin]-5-yl)cyclopropyl)imidazo[1,2-a]pyridine;
5-(2-([2,2′-bipyrimidin]-5-yl)cyclopropyl)quinoline;
5-(2-([2,2′-bipyrimidin]-5-yl)cyclopropyl)-8-fluoroquinoline;
5-(2-([2,2′-bipyrimidin]-5-yl)cyclopropyl)-8-methoxyquinoline;
5-(2-(3,4-difluoro-5-methoxyphenyl)cyclopropyl)-2-(pyridin-2-yl)pyrimidine;
5-(2-(4-fluoro-3,5-dimethoxyphenyl)cyclopropyl)-2-(pyridin-2-yl)pyrimidine;
2-(pyridin-2-yl)-5-(2-(3,4,5-trimethoxyphenyl)cyclopropyl)pyrimidine;
5-(2-(3,4-difluoro-5-methoxyphenyl)cyclopropyl)-2,4′-bipyrimidine;
5-(2-(3,4-difluoro-5-methoxyphenyl)cyclopropyl)-2-(pyrazin-2-yl)pyrimidine;
5-(2-(3,4-difluoro-5-methoxyphenyl)cyclopropyl)-2-(3-fluoropyridin-2-yl)pyrimidine;
5-(2-(3,4-difluoro-5-methoxyphenyl)cyclopropyl)-2-(3-methoxypyridin-2-yl)pyrimidine;
5-(2-(3,4-difluoro-5-(1H-imidazol-1-yl)phenyl)cyclopropyl)-2,2′-bipyrimidine;
5-(2-(5,6-dimethoxypyridin-3-yl)cyclopropyl)-2,2′-bipyrimidine;
5-(2-(3-(difluoromethoxy)-4-fluorophenyl)cyclopropyl)-2,2′-bipyrimidine;
5-(2-(4-fluoro-3-(4-methylpiperazin-1-yl)phenyl)cyclopropyl)-2,2′-bipyrimidine;
5-(2-(2,4-difluoro-3-methoxyphenyl)cyclopropyl)-2,2′-bipyrimidine;
3-(2-(4-fluoro-3-methoxyphenyl)cyclopropyl)-6-(pyrimidin-2-yl)pyridazine;
4-(2-(4-fluoro-3-methoxyphenyl)cyclopropyl)-1-(pyrimidin-2-yl)isoquinoline;
5-(2-(4-fluoro-3-(piperidin-1-yl)phenyl)cyclopropyl)-2,2′-bipyrimidine;
1-(5-(2-([2,2′-bipyrimidin]-5-yl)cyclopropyl)-2-fluorophenyl)pyrrolidin-2-one;
5-(2-(4-chloro-3-(pyrrolidin-1-yl)phenyl)cyclopropyl)-2,2′-bipyrimidine;
5-((2-([2,2′-bipyrimidin]-5-yl)cyclopropyl)-2-chloro-N-methylbenzamide;
5-(2-([2,2′-bipyrimidin]-5-yl)cyclopropyl)-2-chloro-N,N-dimethylbenzamide;
N-(5-(2-([2,2′-bipyrimidin]-5-yl)cyclopropyl)-2-chlorophenyl)acetamide;
5-(2-([2,2′-bipyrimidin]-5-yl)cyclopropyl)-N-cyclopentyl-2,3-difluoroaniline;
6-(5-(2-([2,2′-bipyrimidin]-5-yl)cyclopropyl)-2,3-difluorophenyl)-2-oxa-6-azaspiro[3.3]heptane;
5-(2-(3,4-difluoro-5-(3-isopropoxyazetidin-1-yl)phenyl)cyclopropyl)-2,2′-bipyrimidine;
5-(2-(3-(3-(tert-butyl sulfonyl)azetidin-1-yl)-4,5-difluorophenyl)cyclopropyl)-2,2′-bipyrimidine;
5-(2-(3,4-difluoro-5-(3-(2-methoxyethoxy)azetidin-1-yl)phenyl)cyclopropyl)-2,2′-bipyrimidine;
5-(2-(3-(3-(3,4-difluoro-5-methoxyphenyl)azetidin-1-yl)-4,5-difluorophenyl)cyclopropyl)-2,2′-bipyrimidine;
5-(2-(3-(3-(3,4-difluorophenyl)azetidin-1-yl)-4,5-difluorophenyl)cyclopropyl)-2,2′-bipyrimidine;
5-(2-(3,4-difluoro-5-(3-(4-fluoro-3-methoxyphenyl)azetidin-1-yl)phenyl)cyclopropyl)-2,2′-bipyrimidine;
5-(2-(3-(3-(3,4-dimethoxyphenyl)azetidin-1-yl)-4,5-difluorophenyl)cyclopropyl)-2,2′-bipyrimidine;
5-(2-(3,4-difluoro-5-((1-methyl-1H-1,2,4-triazol-3-yl)methoxy)phenyl)cyclopropyl)-2,2′-bipyrimidine;
2-((5-(2-([2,2′-bipyrimidin]-5-yl)cyclopropyl)-2,3-difluorophenoxy)methyl)thiazole;
5-(2-(3,4-difluoro-5-((1-methyl-1H-pyrazol-3-yl)methoxy)phenyl)cyclopropyl)-2,2′-bipyrimidine;
5-(2-(3-(3,3-dimethylpyrrolidin-1-yl)-4,5-difluorophenyl)cyclopropyl)-2,2′-bipyrimidine;
6-(5-(2-([2,2′-bipyrimidin]-5-yl)cyclopropyl)-2,3-difluorophenyl)-2-oxa-6-azaspiro[3.4]octane;
1-((3S)-3-((5-(2-([2,2′-bipyrimidin]-5-yl)cyclopropyl)-2,3-difluorophenyl)amino)pyrrolidin-1-yl)ethenone;
1-((3R)-3-((5-(2-([2,2′-bipyrimidin]-5-yl)cyclopropyl)-2,3-difluorophenyl) amino) pyrrolidin-1-yl)ethenone;
(3S)-N-(5-(2-([2,2′-bipyrimidin]-5-yl)cyclopropyl)-2,3-difluorophenyl)-1-methylpyrrolidin-3-amine;
(3R)-N-(5-(2-([2,2′-bipyrimidin]-5-yl)cyclopropyl)-2,3-difluorophenyl)-1-methylpyrrolidin-3-amine;
5-(2-(3,4-difluoro-5-(4-methyl-1H-pyrazol-1-yl)phenyl)cyclopropyl)-2,2′-bipyrimidine;
N-(5-(2-([2,2′-bipyrimidin]-5-yl)cyclopropyl)-2,3-difluorophenyl)-N-methyloxetan-3-amine;
(1-(5-(2-([2,2′-bipyrimidin]-5-yl)cyclopropyl)-2,3-difluorophenyl)-1H-pyrazol-4-yl)methanol;
5-(2-(3-((3R,4 S)-3,4-dimethoxypyrrolidin-1-yl)-4,5-difluorophenyl)cyclopropyl)-2,2′-bipyrimidine;
5-(2-(3,4-difluoro-5-(4-(methoxymethyl)-1H-pyrazol-1-yl)phenyl)cyclopropyl)-2,2′-bipyrimidine;
1-(5-(2-([2,2′-bipyrimidin]-5-yl)cyclopropyl)-2,3-difluorophenyl)-5,6-dimethoxy-1H-benzo[d]imidazole;
2-(5-(2-([2,2′-bipyrimidin]-5-yl)cyclopropyl)-2,3-difluorophenyl)-2-azaspiro[3.3]heptan-6-ol;
(3R,4R)-1-(5-(2-([2,2′-bipyrimidin]-5-yl)cyclopropyl)-2,3-difluorophenyl)pyrrolidine-3,4-diol;
4-(3-(5-(2-([2,2′-bipyrimidin]-5-yl)cyclopropyl)-2,3-difluorophenoxy)propyl)morpholine;
4-(3-(4-(2-([2,2′-bipyrimidin]-5-yl)cyclopropyl)-2,6-difluorophenoxy)propyl)morpholine;
5-(2-(4-((2-oxaspiro[3.3]heptan-6-yl)oxy)-3,5-difluorophenyl)cyclopropyl)-2,2′-bipyrimidine;
5-(2-(3-((2-oxaspiro[3.3]heptan-6-yl)oxy)-4,5-difluorophenyl)cyclopropyl)-2,2′-bipyrimidine;
2-((5-(2-([2,2′-bipyrimidin]-5-yl)cyclopropyl)-2,3-difluorophenyl)(methyl)amino)ethan-1-ol;
5-(2-(3-(cyclopentyloxy)-4,5-difluorophenyl)cyclopropyl)-2,2′-bipyrimidine;
5-(2-(3,4-difluoro-5-(((R)-tetrahydrofuran-3-yl)oxy)phenyl)cyclopropyl)-2,2′-bipyrimidine;
5-(2-(3,4-difluoro-5-(((S)-tetrahydrofuran-3-yl)oxy)phenyl)cyclopropyl)-2,2′-bipyrimidine;
5-(2-(3,4-difluoro-5-(4-methoxy-1H-pyrazol-1-yl)phenyl)cyclopropyl)-2,2′-bipyrimidine;
5-(2-(3,4-difluoro-5-(1H-pyrazol-1-yl)phenyl)cyclopropyl)-2,2′-bipyrimidine;
5-(2-(3,4-difluoro-5-(3-phenylazetidin-1-yl)phenyl)cyclopropyl)-2,2′-bipyrimidine;
5-(2-(3-(3,4-difluoro-1H-pyrrol-1-yl)-4,5-difluorophenyl)cyclopropyl)-2,2′-bipyrimidine;
1-(5-(2-([2,2′-bipyrimidin]-5-yl)cyclopropyl)-2,3-difluorophenyl)-1H-pyrazol-4-ol;
5-(2-(3,4-difluoro-5-(4-methyl-1H-imidazol-1-yl)phenyl)cyclopropyl)-2,2′-bipyrimidine;
ethyl 2-([2,2′-bipyrimidin]-5-yl)-3-(3,4-difluoro-5-methoxyphenyl)cyclopropane-1-carboxylate;
4-(2-([2,2′-bipyrimidin]-5-yl)cyclopropyl)-6,7-difluoro-1-(3-methoxypropyl)-1H-indazole;
6-(2-([2,2′-bipyrimidin]-5-yl)cyclopropyl)-4-fluoro-2-(3-methoxypropyl)-2H-indazole;
2-(2-([2,2′-bipyrimidin]-5-yl)cyclopropyl)-4,6-difluorobenzo[d]thiazole;
6-(2-([2,2′-bipyrimidin]-5-yl)cyclopropyl)-4-fluoro-1-isopropyl-2-methyl-1H-benzo[d]imidazole;
6-(2-([2,2′-bipyrimidin]-5-yl)cyclopropyl)-4-fluoro-2-methylbenzo[d]thiazole;
5-(2-([2,2′-bipyrimidin]-5-yl)cyclopropyl)-7-fluoro-1-(3-methoxypropyl)-1H-benzo[d]imidazole;
6-(2-([2,2′-bipyrimidin]-5-yl)cyclopropyl)-4-fluoro-1-(3-methoxypropyl)-1H-benzo[d]imidazole;
6-(2-([2,2′-bipyrimidin]-5-yl)cyclopropyl)-4-fluoro-1-(3-methoxypropyl)-1H-indazole;
4-(2-([2,2′-bipyrimidin]-5-yl)cyclopropyl)-7-fluoro-1-(3-methoxypropyl)-1H-indazole;
4-(2-([2,2′-bipyrimidin]-5-yl)cyclopropyl)-7-fluoro-2-(3-methoxypropyl)-2H-indazole;
5-(2-([2,2′-bipyrimidin]-5-yl)cyclopropyl)-2-methylbenzo[d]thiazole.

In certain embodiments, the compound, or a salt, solvate, isotopically labeled, stereoisomer, any mixture of stereoisomers, tautomer, and/or any mixture of tautomers thereof, is one of the following:
trans-5-(2-(2-fluoro-4-methoxyphenyl)cyclopropyl)-2,2′-bipyrimidine;
trans-4-(2-(3,4-difluorophenyl)cyclopropyl)-2,2′-bipyrimidine;
trans-5-(2-(4-fluoro-2-methoxyphenyl)cyclopropyl)-2,2′-bipyrimidine;
trans-5-(2-(4-fluoro-3-methoxyphenyl)cyclopropyl)-2,2′-bipyrimidine;
trans-5-(2-(3-fluoro-4-methoxyphenyl)cyclopropyl)-2,2′-bipyrimidine;
trans-5-(2-(3,4-difluorophenyl)cyclopropyl)-2,2′-bipyrimidine;
trans-5-(2-(4-fluoro-3-methoxyphenyl)cyclopropyl)-4-methyl-2,2′-bipyrimidine;
trans-5-(2-(4-fluoro-3-(pyrrolidin-1-yl)phenyl)cyclopropyl)-2,2′-bipyrimidine;
trans-5-(2-(4-fluoro-3-(2-methoxyethoxy)phenyl)cyclopropyl)-2,2′-bipyrimidine;
trans-5-(2-(3-fluoro-4-(2-methoxyethoxy)phenyl)cyclopropyl)-2,2′-bipyrimidine;
trans-5-(2-(3-(cyclopropylmethoxy)-4-fluorophenyl)cyclopropyl)-2,2′-bipyrimidine;
trans-5-(2-(3-(2,2-difluoroethoxy)-4-fluorophenyl)cyclopropyl)-2,2′-bipyrimidine;
trans-5-(2-(3-chloro-4-fluorophenyl)cyclopropyl)-2,2′-bipyrimidine;
trans-5-(2-(3,4-dimethoxyphenyl)cyclopropyl)-2,2′-bipyrimidine;
trans-5-(2-(4-chloro-3-methoxyphenyl)cyclopropyl)-2,2′-bipyrimidine;
trans-5-(2-(2-ethyl-4-fluoro-5-methoxyphenyl)cyclopropyl)-2,2′-bipyrimidine;
trans-5-(2-(4-fluoro-5-methoxy-2-propylphenyl)cyclopropyl)-2,2′-bipyrimidine;
trans-5-(2-(4-fluoro-3-(trifluoromethoxy)phenyl)cyclopropyl)-2,2′-bipyrimidine;
trans-5-(2-(4-fluoro-3-(4-(methylsulfonyl)piperazin-1-yl)phenyl)cyclopropyl)-2,2′-bipyrimidine;
trans-5-(2-(3-(3,3-difluoropyrrolidin-1-yl)-4-fluorophenyl)cyclopropyl)-2,2′-bipyrimidine;
trans-5-(2-(4-chloro-3-fluoro-5-methoxyphenyl)cyclopropyl)-2,2′-bipyrimidine;
trans-5-(2-(4-fluoro-3-(3-methoxyazetidin-1-yl)phenyl)cyclopropyl)-2,2′-bipyrimidine;
trans-5-((2-(4-chloro-3-(cyclopropylmethoxy)-5-methylphenyl)cyclopropyl)-2,2′-bipyrimidine;
trans-5-(2-(4-chloro-5-methoxy-2-methylphenyl)cyclopropyl)-2,2′-bipyrimidine;
trans-1-(5-(2-([2,2′-bipyrimidin]-5-yl)cyclopropyl)-2-fluorophenyl)azetidin-3-ol;
trans-5-(2-(4-chloro-2-fluoro-3-methoxyphenyl)cyclopropyl)-2,2′-bipyrimidine;
trans-5-(2-(4-chloro-3-methoxy-5-methylphenyl)cyclopropyl)-2,2′-bipyrimidine;
trans-5-(2-(3,4-dichloro-5-methoxyphenyl)cyclopropyl)-2,2′-bipyrimidine;
trans-5-(2-(4-chloro-3-(cyclopropylmethoxy)-2-methylphenyl)cyclopropyl)-2,2′-bipyrimidine;
trans-5-(2-([2,2′-bipyrimidin]-5-yl)cyclopropyl)-2-fluoro-N,N-dimethylaniline;
trans-(3S)-1-(5-(2-([2,2′-bipyrimidin]-5-yl)cyclopropyl)-2-fluorophenyl)pyrrolidin-3-ol;
trans-5-(2-(4-fluoro-3-((S)-3-methoxypyrrolidin-1-yl)phenyl)cyclopropyl)-2,2′-bipyrimidine;
trans-(3R)-1-(5-(2-([2,2′-bipyrimidin]-5-yl)cyclopropyl)-2-fluorophenyl)pyrrolidin-3-ol;
trans-5-(2-(4-chloro-3-methoxy-2-methylphenyl)cyclopropyl)-2,2′-bipyrimidine;
trans-5-(2-(4-fluoro-3-methoxyphenyl)cyclopropyl)-4-isopropyl-2,2′-bipyrimidine;
trans-4-ethyl-5-(2-(4-fluoro-3-methoxyphenyl)cyclopropyl)-2,2′-bipyrimidine;
trans-4-cyclohexyl-5-(2-(4-fluoro-3-methoxyphenyl)cyclopropyl)-2,2′-bipyrimidine;
trans-5-(2-(4-fluoro-3-methoxyphenyl)cyclopropyl)-4-methoxy-2,2′-bipyrimidine;
trans-5-(2-(3-(azetidin-1-yl)-4-fluorophenyl)cyclopropyl)-2,2′-bipyrimidine;
trans-5-(2-(3,4-difluoro-5-methoxyphenyl)cyclopropyl)-2,2′-bipyrimidine;
trans-5-(2-(4-chloro-2-fluoro-5-methoxyphenyl)cyclopropyl)-2,2′-bipyrimidine;
trans-5-(2-(2,4-dichloro-5-methoxyphenyl)cyclopropyl)-2,2′-bipyrimidine;
trans-5-(2-(4-chloro-3-fluoro-5-(pyrrolidin-1-yl)phenyl)cyclopropyl)-2,2′-bipyrimidine;
trans-5-(2-(3,4-difluoro-5-(pyrrolidin-1-yl)phenyl)cyclopropyl)-2,2′-bipyrimidine;
trans-5-(2-(4-fluoro-3-((R)-3-methoxypyrrolidin-1-yl)phenyl)cyclopropyl)-2,2′-bipyrimidine;
trans-5-(2-(3,4-difluoro-5-((R)-3-methoxypyrrolidin-1-yl)phenyl)cyclopropyl)-2,2′-bipyrimidine;
trans-5-(2-(4-chloro-3,5-dimethoxyphenyl)cyclopropyl)-2,2′-bipyrimidine;
trans-5-(2-(4-fluoro-3-methoxy-5-(pyrrolidin-1-yl)phenyl)cyclopropyl)-2,2′-bipyrimidine;
trans-5-(2-(3,4-difluoro-5-((S)-3-methoxypyrrolidin-1-yl)phenyl)cyclopropyl)-2,2′-bipyrimidine;
trans-(3R)-1-(5-(2-([2,2′-bipyrimidin]-5-yl)cyclopropyl)-2,3-difluorophenyl)pyrrolidin-3-ol;
trans-(3S)-1-(5-(2-([2,2′-bipyrimidin]-5-yl)cyclopropyl)-2,3-difluorophenyl)pyrrolidin-3-ol;
trans-5-(2-(3-chloro-4-fluoro-5-(pyrrolidin-1-yl)phenyl)cyclopropyl)-2,2′-bipyrimidine;
trans-5-(2-(3-chloro-4-fluoro-5-((R)-3-methoxypyrrolidin-1-yl)phenyl)cyclopropyl)-2,2′-bipyrimidine;
trans-5-(2-(3-fluoro-5-methoxyphenyl)cyclopropyl)-2,2′-bipyrimidine;
trans-5-(2-(4-chloro-2,3-dimethoxyphenyl)cyclopropyl)-2,2′-bipyrimidine;
trans-5-(2-(3-chloro-4-fluoro-5-((S)-3-methoxypyrrolidin-1-yl)phenyl)cyclopropyl)-2,2′-bipyrimidine;
trans-5-(2-(3-chloro-5-fluorophenyl)cyclopropyl)-2,2′-bipyrimidine;
trans-(3S)-1-(5-(2-([2,2′-bipyrimidin]-5-yl)cyclopropyl)-3-chloro-2-fluorophenyl)pyrrolidin-3-ol;
trans-5-(2-(3,4-difluoro-5-(3-methoxyazetidin-1-yl)phenyl)cyclopropyl)-2,2′-bipyrimidine;
trans-2-(5-(2-(3,4-difluoro-5-methoxyphenyl)cyclopropyl)pyridin-2-yl)pyrimidine;
trans-(3R)-1-(5-(2-([2,2′-bipyrimidin]-5-yl)cyclopropyl)-3-chloro-2-fluorophenyl)pyrrolidin-3-ol;
trans-5-(2-(3-(cyclopropylmethoxy)-4,5-difluorophenyl)cyclopropyl)-2,2′-bipyrimidine;
trans-5-(2-(3,4-difluoro-5-(3-methoxypropoxy)phenyl)cyclopropyl)-2,2′-bipyrimidine;
trans-5-(2-(2,4-dichloro-3-methoxyphenyl)cyclopropyl)-2,2′-bipyrimidine;
trans-5-(2-(3,4-difluoro-5-(2-methoxyethoxy)phenyl)cyclopropyl)-2,2′-bipyrimidine;
trans-2-(5-(2-([2,2′-bipyrimidin]-5-yl)cyclopropyl)-2,3-difluorophenyl)-7-oxa-2-azaspiro[3.5]nonane;
trans-5-(2-(3-(3,3-dimethylazetidin-1-yl)-4,5-difluorophenyl)cyclopropyl)-2,2′-bipyrimidine;
trans-6-(5-(2-(4-fluoro-3-methoxyphenyl)cyclopropyl)-[2,2′-bipyrimidin]-4-yl)-2-methylbenzo[d]thiazole;
trans-5-(2-(4-Fluoro-3-methoxyphenyl)cyclopropyl)-4-phenyl-2,2′-bipyrimidine;
trans-5-(2-(4-fluoro-3-methoxyphenyl)cyclopropyl)-4-(piperidin-1-yl)-2,2′-bipyrimidine;
trans-5-(2-(4-fluoro-3,5-dimethoxyphenyl)cyclopropyl)-2,2′-bipyrimidine;
trans-5-(2-(3-chloro-4-fluoro-5-methoxyphenyl)cyclopropyl)-2,2′-bipyrimidine;
trans-5-(2-(4-fluoro-3-methoxy-5-methylphenyl)cyclopropyl)-2,2′-bipyrimidine;
trans-5-(2-(3-methoxy-4-(trifluoromethoxy)phenyl)cyclopropyl)-2,2′-bipyrimidine;
trans-5-(2-(3-methoxy-4-(trifluoromethyl)phenyl)cyclopropyl)-2,2′-bipyrimidine;
trans-5-(2-(5-chloro-4-fluoro-2-(3-methoxypropoxy)phenyl)cyclopropyl)-2,2′-bipyrimidine;
trans-5-(2-(5-chloro-4-fluoro-2-(2-methoxyethoxy)phenyl)cyclopropyl)-2,2′-bipyrimidine;
trans-5-(2-(5-chloro-4-methoxy-[1,1′-biphenyl]-2-yl)cyclopropyl)-2,2′-bipyrimidine;
trans-5-(2-(3,4,5-trifluorophenyl)cyclopropyl)-2,2′-bipyrimidine;
trans-5-(2-(3-methoxy-5-(trifluoromethyl)phenyl)cyclopropyl)-2,2′-bipyrimidine;
trans-5-(2-(3-methoxy-5-(trifluoromethoxy)phenyl)cyclopropyl)-2,2′-bipyrimidine;
trans-5-(2-(4-fluoro-3-methoxy-5-(trifluoromethyl)phenyl)cyclopropyl)-2,2′-bipyrimidine;
trans-5-(2-(naphthalen-1-yl)cyclopropyl)-2,2′-bipyrimidine;
trans-5-(2-(naphthalen-2-yl)cyclopropyl)-2,2′-bipyrimidine;
trans-5-(2-(4-fluoronaphthalen-1-yl)cyclopropyl)-2,2′-bipyrimidine;
trans-5-(2-(4-fluoronaphthalen-2-yl)cyclopropyl)-2,2′-bipyrimidine;
trans-3-(2-([2,2′-bipyrimidin]-5-yl)cyclopropyl)imidazo[1,2-a]pyridine;
trans-5-(2-([2,2′-bipyrimidin]-5-yl)cyclopropyl)quinoline;
trans-5-(2-([2,2′-bipyrimidin]-5-yl)cyclopropyl)-8-fluoroquinoline;
trans-5-(2-([2,2′-bipyrimidin]-5-yl)cyclopropyl)-8-methoxyquinoline;
trans-5-(2-(3,4-difluoro-5-methoxyphenyl)cyclopropyl)-2-(pyridin-2-yl)pyrimidine;
trans-5-(2-(4-fluoro-3,5-dimethoxyphenyl)cyclopropyl)-2-(pyridin-2-yl)pyrimidine;
trans-2-(pyridin-2-yl)-5-(2-(3,4,5-trimethoxyphenyl)cyclopropyl)pyrimidine;
trans-5-(2-(3,4-difluoro-5-methoxyphenyl)cyclopropyl)-2,4′-bipyrimidine;
trans-5-(2-(3,4-difluoro-5-methoxyphenyl)cyclopropyl)-2-(pyrazin-2-yl)pyrimidine;
trans-5-(2-(3,4-difluoro-5-methoxyphenyl)cyclopropyl)-2-(3-fluoropyridin-2-yl)pyrimidine;
trans-5-(2-(3,4-difluoro-5-methoxyphenyl)cyclopropyl)-2-(3-methoxypyridin-2-yl)pyrimidine;
trans-5-(2-(3,4-difluoro-5-(1H-imidazol-1-yl)phenyl)cyclopropyl)-2,2′-bipyrimidine;
trans-5-(2-(5,6-dimethoxypyridin-3-yl)cyclopropyl)-2,2′-bipyrimidine;
trans-5-(2-(3-(difluoromethoxy)-4-fluorophenyl)cyclopropyl)-2,2′-bipyrimidine;
trans-5-(2-(4-fluoro-3-(4-methylpiperazin-1-yl)phenyl)cyclopropyl)-2,2′-bipyrimidine;
trans-5-(2-(2,4-difluoro-3-methoxyphenyl)cyclopropyl)-2,2′-bipyrimidine;
trans-3-(2-(4-fluoro-3-methoxyphenyl)cyclopropyl)-6-(pyrimidin-2-yl)pyridazine;
trans-4-(2-(4-fluoro-3-methoxyphenyl)cyclopropyl)-1-(pyrimidin-2-yl)isoquinoline;
trans-5-(2-(4-fluoro-3-(piperidin-1-yl)phenyl)cyclopropyl)-2,2′-bipyrimidine;
trans-1-(5-(2-([2,2′-bipyrimidin]-5-yl)cyclopropyl)-2-fluorophenyl)pyrrolidin-2-one;
trans-5-(2-(4-chloro-3-(pyrrolidin-1-yl)phenyl)cyclopropyl)-2,2′-bipyrimidine;
trans-5-((2-([2,2′-bipyrimidin]-5-yl)cyclopropyl)-2-chloro-N-methylbenzamide;
trans-5-(2-([2,2′-bipyrimidin]-5-yl)cyclopropyl)-2-chloro-N,N-dimethylbenzamide;
trans-N-(5-(2-([2,2′-bipyrimidin]-5-yl)cyclopropyl)-2-chlorophenyl)acetamide;
trans-5-(2-([2,2′-bipyrimidin]-5-yl)cyclopropyl)-N-cyclopentyl-2,3-difluoroaniline;
trans-6-(5-(2-([2,2′-bipyrimidin]-5-yl)cyclopropyl)-2,3-difluorophenyl)-2-oxa-6-azaspiro[3.3]heptane;
trans-5-(2-(3,4-difluoro-5-(3-isopropoxyazetidin-1-yl)phenyl)cyclopropyl)-2,2′-bipyrimidine;
trans-5-(2-(3-(3-(tert-butyl sulfonyl)azetidin-1-yl)-4,5-difluorophenyl)cyclopropyl)-2,2′-bipyrimidine;
trans-5-(2-(3,4-difluoro-5-(3-(2-methoxyethoxy)azetidin-1-yl)phenyl)cyclopropyl)-2,2′-bipyrimidine;
trans-5-(2-(3-(3-(3,4-difluoro-5-methoxyphenyl)azetidin-1-yl)-4,5-difluorophenyl)cyclopropyl)-2,2′-bipyrimidine;
trans-5-(2-(3-(3-(3,4-difluorophenyl)azetidin-1-yl)-4,5-difluorophenyl)cyclopropyl)-2,2′-bipyrimidine;
trans-5-(2-(3,4-difluoro-5-(3-(4-fluoro-3-methoxyphenyl)azetidin-1-yl)phenyl)cyclopropyl)-2,2′-bipyrimidine;
trans-5-(2-(3-(3-(3,4-dimethoxyphenyl)azetidin-1-yl)-4,5-difluorophenyl)cyclopropyl)-2,2′-bipyrimidine;
trans-5-(2-(3,4-difluoro-5-((1-methyl-1H-1,2,4-triazol-3-yl)methoxy)phenyl)cyclopropyl)-2,2′-bipyrimidine;
trans-2((5-(2-([2,2′-bipyrimidin]-5-yl)cyclopropyl)-2,3-difluorophenoxy)methyl)thiazole;
trans-5-(2-(3,4-difluoro-5-(1-methyl-1H-pyrazol-3-yl)methoxy)phenyl)cyclopropyl)-2,2′-bipyrimidine;
trans-5-(2-(3-(3,3-dimethylpyrrolidin-1-yl)-4,5-difluorophenyl)cyclopropyl)-2,2′-bipyrimidine;
trans-6-(5-(2-([2,2′-bipyrimidin]-5-yl)cyclopropyl)-2,3-difluorophenyl)-2-oxa-6-azaspiro[3.4]octane;
trans-1-((3S)-3-((5-(2-([2,2′-bipyrimidin]-5-yl)cyclopropyl)-2,3-difluorophenyl)amino)pyrrolidin-1-yl)ethenone;
trans-1-((3R)-3-((5-(2-([2,2′-bipyrimidin]-5-yl)cyclopropyl)-2,3-difluorophenyl) amino) pyrrolidin-1-yl)ethenone;
trans-(3S)-N-(5-(2-([2,2′-bipyrimidin]-5-yl)cyclopropyl)-2,3-difluorophenyl)-1-methylpyrrolidin-3-amine;
trans-(3R)-N-(5-(2-([2,2′-bipyrimidin]-5-yl)cyclopropyl)-2,3-difluorophenyl)-1-methylpyrrolidin-3-amine;
trans-5-(2-(3,4-difluoro-5-(4-methyl-1H-pyrazol-1-yl)phenyl)cyclopropyl)-2,2′-bipyrimidine;
trans-N-(5-(2-([2,2′-bipyrimidin]-5-yl)cyclopropyl)-2,3-difluorophenyl)-N-methyloxetan-3-amine;
trans-(1-(5-(2-([2,2′-bipyrimidin]-5-yl)cyclopropyl)-2,3-difluorophenyl)-1H-pyrazol-4-yl)methanol;
trans-5-(2-(3-((3R,4S)-3,4-dimethoxypyrrolidin-1-yl)-4,5-difluorophenyl)cyclopropyl)-2,2′-bipyrimidine;
trans-5-(2-(3,4-difluoro-5-(4-(methoxymethyl)-1H-pyrazol-1-yl)phenyl)cyclopropyl)-2,2′-bipyrimidine;
trans-1-(5-(2-([2,2′-bipyrimidin]-5-yl)cyclopropyl)-2,3-difluorophenyl)-5,6-dimethoxy-1H-benzo[d]imidazole;
trans-2-(5-(2-([2,2′-bipyrimidin]-5-yl)cyclopropyl)-2,3-difluorophenyl)-2-azaspiro[3.3]heptan-6-ol;
trans-(3R,4R)-1-(5-(2-([2,2′-bipyrimidin]-5-yl)cyclopropyl)-2,3-difluorophenyl)pyrrolidine-3,4-diol;
trans-4-(3-(5-(2-([2,2′-bipyrimidin]-5-yl)cyclopropyl)-2,3-difluorophenoxy)propyl)morpholine;
trans-4-(3-(4-(2-([2,2′-bipyrimidin]-5-yl)cyclopropyl)-2,6-difluorophenoxy)propyl)morpholine;
trans-5-(2-(4-((2-oxaspiro[3.3]heptan-6-yl)oxy)-3,5-difluorophenyl)cyclopropyl)-2,2′-bipyrimidine;
trans-5-(2-(3-((2-oxaspiro[3.3]heptan-6-yl)oxy)-4,5-difluorophenyl)cyclopropyl)-2,2′-bipyrimidine;
trans-2-((5-(2-([2,2′-bipyrimidin]-5-yl)cyclopropyl)-2,3-difluorophenyl)(methyl)amino)ethan-1-ol;
trans-5-(2-(3-(cyclopentyloxy)-4,5-difluorophenyl)cyclopropyl)-2,2′-bipyrimidine;
trans-5-(2-(3,4-difluoro-5-((R)-tetrahydrofuran-3-yl)oxy)phenyl)cyclopropyl)-2,2′-bipyrimidine;
trans-5-(2-(3,4-difluoro-5-((S)-tetrahydrofuran-3-yl)oxy)phenyl)cyclopropyl)-2,2′-bipyrimidine;
trans-5-(2-(3,4-difluoro-5-(4-methoxy-1H-pyrazol-1-yl)phenyl)cyclopropyl)-2,2′-bipyrimidine;
trans-5-(2-(3,4-difluoro-5-(1H-pyrazol-1-yl)phenyl)cyclopropyl)-2,2′-bipyrimidine;
trans-5-(2-(3,4-difluoro-5-(3-phenylazetidin-1-yl)phenyl)cyclopropyl)-2,2′-bipyrimidine;
trans-5-(2-(3-(3,4-difluoro-1H-pyrrol-1-yl)-4,5-difluorophenyl)cyclopropyl)-2,2′-bipyrimidine;
trans-1-(5-(2-([2,2′-bipyrimidin]-5-yl)cyclopropyl)-2,3-difluorophenyl)-1H-pyrazol-4-ol;
trans-5-(2-(3,4-difluoro-5-(4-methyl-1H-imidazol-1-yl)phenyl)cyclopropyl)-2,2′-bipyrimidine;
trans-ethyl 2-([2,2′-bipyrimidin]-5-yl)-3-(3,4-difluoro-5-methoxyphenyl)cyclopropane-1-carboxylate;
trans-4-(2-([2,2′-bipyrimidin]-5-yl)cyclopropyl)-6,7-difluoro-1-(3-methoxypropyl)-1H-indazole;
trans-6-(2-([2,2′-bipyrimidin]-5-yl)cyclopropyl)-4-fluoro-2-(3-methoxypropyl)-2H-indazole;
trans-2-(2-([2,2′-bipyrimidin]-5-yl)cyclopropyl)-4,6-difluorobenzo[d]thiazole;
trans-6-(2-([2,2′-bipyrimidin]-5-yl)cyclopropyl)-4-fluoro-1-isopropyl-2-methyl-1H-benzo[d]imidazole;
trans-6-(2-([2,2′-bipyrimidin]-5-yl)cyclopropyl)-4-fluoro-2-methylbenzo[d]thiazole;
trans-5-(2-([2,2′-bipyrimidin]-5-yl)cyclopropyl)-7-fluoro-1-(3-methoxypropyl)-1H-benzo[d]imidazole;
trans-6-(2-([2,2′-bipyrimidin]-5-yl)cyclopropyl)-4-fluoro-1-(3-methoxypropyl)-1H-benzo[d]imidazole;
trans-6-(2-([2,2′-bipyrimidin]-5-yl)cyclopropyl)-4-fluoro-1-(3-methoxypropyl)-1H-indazole;
trans-4-(2-([2,2′-bipyrimidin]-5-yl)cyclopropyl)-7-fluoro-1-(3-methoxypropyl)-1H-indazole;
trans-4-(2-([2,2′-bipyrimidin]-5-yl)cyclopropyl)-7-fluoro-2-(3-methoxypropyl)-2H-indazole;
trans-5-(2-([2,2′-bipyrimidin]-5-yl)cyclopropyl)-2-methylbenzo[d]thiazole.

In certain embodiments, the compound, or a salt, solvate, isotopically labeled, stereoisomer, any mixture of stereoisomers, tautomer, and/or any mixture of tautomers thereof, is one of the following:
cis-5-(2-(2-fluoro-4-methoxyphenyl)cyclopropyl)-2,2′-bipyrimidine;
cis-4-(2-(3,4-difluorophenyl)cyclopropyl)-2,2′-bipyrimidine;
cis-5-(2-(4-fluoro-2-methoxyphenyl)cyclopropyl)-2,2′-bipyrimidine;
cis-5-(2-(4-fluoro-3-methoxyphenyl)cyclopropyl)-2,2′-bipyrimidine;
cis-5-(2-(3-fluoro-4-methoxyphenyl)cyclopropyl)-2,2′-bipyrimidine;
cis-5-(2-(3,4-difluorophenyl)cyclopropyl)-2,2′-bipyrimidine;
cis-5-(2-(4-fluoro-3-methoxyphenyl)cyclopropyl)-4-methyl-2,2′-bipyrimidine;
cis-5-(2-(4-fluoro-3-(pyrrolidin-1-yl)phenyl)cyclopropyl)-2,2′-bipyrimidine;
cis-5-(2-(4-fluoro-3-(2-methoxyethoxy)phenyl)cyclopropyl)-2,2′-bipyrimidine;
cis-5-(2-(3-fluoro-4-(2-methoxyethoxy)phenyl)cyclopropyl)-2,2′-bipyrimidine;
cis-5-(2-(3-(cyclopropylmethoxy)-4-fluorophenyl)cyclopropyl)-2,2′-bipyrimidine;
cis-5-(2-(3-(2,2-difluoroethoxy)-4-fluorophenyl)cyclopropyl)-2,2′-bipyrimidine;
cis-5-(2-(3-chloro-4-fluorophenyl)cyclopropyl)-2,2′-bipyrimidine;
cis-5-(2-(3,4-dimethoxyphenyl)cyclopropyl)-2,2′-bipyrimidine;
cis-5-(2-(4-chloro-3-methoxyphenyl)cyclopropyl)-2,2′-bipyrimidine;
cis-5-(2-(2-ethyl-4-fluoro-5-methoxyphenyl)cyclopropyl)-2,2′-bipyrimidine;
cis-5-(2-(4-fluoro-5-methoxy-2-propylphenyl)cyclopropyl)-2,2′-bipyrimidine;
cis-5-(2-(4-fluoro-3-(trifluoromethoxy)phenyl)cyclopropyl)-2,2′-bipyrimidine;
cis-5-(2-(4-fluoro-3-(4-(methylsulfonyl)piperazin-1-yl)phenyl)cyclopropyl)-2,2′-bipyrimidine;
cis-5-(2-(3-(3,3-difluoropyrrolidin-1-yl)-4-fluorophenyl)cyclopropyl)-2,2′-bipyrimidine;
cis-5-(2-(4-chloro-3-fluoro-5-methoxyphenyl)cyclopropyl)-2,2′-bipyrimidine;
cis-5-(2-(4-fluoro-3-(3-methoxyazetidin-1-yl)phenyl)cyclopropyl)-2,2′-bipyrimidine;
cis-5-((2-(4-chloro-3-(cyclopropylmethoxy)-5-methylphenyl)cyclopropyl)-2,2′-bipyrimidine;
cis-5-(2-(4-chloro-5-methoxy-2-methylphenyl)cyclopropyl)-2,2′-bipyrimidine;
cis-1-(5-(2-([2,2′-bipyrimidin]-5-yl)cyclopropyl)-2-fluorophenyl)azetidin-3-ol;
cis-5-(2-(4-chloro-2-fluoro-3-methoxyphenyl)cyclopropyl)-2,2′-bipyrimidine;
cis-5-(2-(4-chloro-3-methoxy-5-methylphenyl)cyclopropyl)-2,2′-bipyrimidine;
cis-5-(2-(3,4-dichloro-5-methoxyphenyl)cyclopropyl)-2,2′-bipyrimidine;
cis-5-(2-(4-chloro-3-(cyclopropylmethoxy)-2-methylphenyl)cyclopropyl)-2,2′-bipyrimidine;
cis-5-(2-([2,2′-bipyrimidin]-5-yl)cyclopropyl)-2-fluoro-N,N-dimethylaniline;
cis-(3S)-1-(5-(2-([2,2′-bipyrimidin]-5-yl)cyclopropyl)-2-fluorophenyl)pyrrolidin-3-ol;
cis-5-(2-(4-fluoro-3-((S)-3-methoxypyrrolidin-1-yl)phenyl)cyclopropyl)-2,2′-bipyrimidine;
cis-(3R)-1-(5-(2-([2,2′-bipyrimidin]-5-yl)cyclopropyl)-2-fluorophenyl)pyrrolidin-3-ol;
cis-5-(2-(4-chloro-3-methoxy-2-methylphenyl)cyclopropyl)-2,2′-bipyrimidine;
cis-5-(2-(4-fluoro-3-methoxyphenyl)cyclopropyl)-4-isopropyl-2,2′-bipyrimidine;
cis-4-ethyl-5-(2-(4-fluoro-3-methoxyphenyl)cyclopropyl)-2,2′-bipyrimidine;
cis-4-cyclohexyl-5-(2-(4-fluoro-3-methoxyphenyl)cyclopropyl)-2,2′-bipyrimidine;
cis-5-(2-(4-fluoro-3-methoxyphenyl)cyclopropyl)-4-methoxy-2,2′-bipyrimidine;
cis-5-(2-(3-(azetidin-1-yl)-4-fluorophenyl)cyclopropyl)-2,2′-bipyrimidine;
cis-5-(2-(3,4-difluoro-5-methoxyphenyl)cyclopropyl)-2,2′-bipyrimidine;
cis-5-(2-(4-chloro-2-fluoro-5-methoxyphenyl)cyclopropyl)-2,2′-bipyrimidine;
cis-5-(2-(2,4-dichloro-5-methoxyphenyl)cyclopropyl)-2,2′-bipyrimidine;
cis-5-(2-(4-chloro-3-fluoro-5-(pyrrolidin-1-yl)phenyl)cyclopropyl)-2,2′-bipyrimidine;
cis-5-(2-(3,4-difluoro-5-(pyrrolidin-1-yl)phenyl)cyclopropyl)-2,2′-bipyrimidine;
cis-5-(2-(4-fluoro-3-((R)-3-methoxypyrrolidin-1-yl)phenyl)cyclopropyl)-2,2′-bipyrimidine;
cis-5-(2-(3,4-difluoro-5-((R)-3-methoxypyrrolidin-1-yl)phenyl)cyclopropyl)-2,2′-bipyrimidine;
cis-5-(2-(4-chloro-3,5-dimethoxyphenyl)cyclopropyl)-2,2′-bipyrimidine;
cis-5-(2-(4-fluoro-3-methoxy-5-(pyrrolidin-1-yl)phenyl)cyclopropyl)-2,2′-bipyrimidine;
cis-5-(2-(3,4-difluoro-5-((S)-3-methoxypyrrolidin-1-yl)phenyl)cyclopropyl)-2,2′-bipyrimidine;
cis-(3R)-1-(5-(2-([2,2′-bipyrimidin]-5-yl)cyclopropyl)-2,3-difluorophenyl)pyrrolidin-3-ol;
cis-(3S)-1-(5-(2-([2,2′-bipyrimidin]-5-yl)cyclopropyl)-2,3-difluorophenyl)pyrrolidin-3-ol;
cis-5-(2-(3-chloro-4-fluoro-5-(pyrrolidin-1-yl)phenyl)cyclopropyl)-2,2′-bipyrimidine;
cis-5-(2-(3-chloro-4-fluoro-5-((R)-3-methoxypyrrolidin-1-yl)phenyl)cyclopropyl)-2,2′-bipyrimidine;
cis-5-(2-(3-fluoro-5-methoxyphenyl)cyclopropyl)-2,2′-bipyrimidine;
cis-5-(2-(4-chloro-2,3-dimethoxyphenyl)cyclopropyl)-2,2′-bipyrimidine;
cis-5-(2-(3-chloro-4-fluoro-5-((S)-3-methoxypyrrolidin-1-yl)phenyl)cyclopropyl)-2,2′-bipyrimidine;
cis-5-(2-(3-chloro-5-fluorophenyl)cyclopropyl)-2,2′-bipyrimidine;
cis-(3S)-1-(5-(2-([2,2′-bipyrimidin]-5-yl)cyclopropyl)-3-chloro-2-fluorophenyl)pyrrolidin-3-ol;
cis-5-(2-(3,4-difluoro-5-(3-methoxyazetidin-1-yl)phenyl)cyclopropyl)-2,2′-bipyrimidine;
cis-2-(5-(2-(3,4-difluoro-5-methoxyphenyl)cyclopropyl)pyridin-2-yl)pyrimidine;
cis-(3R)-1-(5-(2-([2,2′-bipyrimidin]-5-yl)cyclopropyl)-3-chloro-2-fluorophenyl)pyrrolidin-3-ol;
cis-5-(2-(3-(cyclopropylmethoxy)-4,5-difluorophenyl)cyclopropyl)-2,2′-bipyrimidine;
cis-5-(2-(3,4-difluoro-5-(3-methoxypropoxy)phenyl)cyclopropyl)-2,2′-bipyrimidine;
cis-5-(2-(2,4-dichloro-3-methoxyphenyl)cyclopropyl)-2,2′-bipyrimidine;
cis-5-(2-(3,4-difluoro-5-(2-methoxyethoxy)phenyl)cyclopropyl)-2,2′-bipyrimidine;
cis-2-(5-(2-([2,2′-bipyrimidin]-5-yl)cyclopropyl)-2,3-difluorophenyl)-7-oxa-2-azaspiro[3.5]nonane;
cis-5-(2-(3-(3,3-dimethylazetidin-1-yl)-4,5-difluorophenyl)cyclopropyl)-2,2′-bipyrimidine;
cis-6-(5-(2-(4-fluoro-3-methoxyphenyl)cyclopropyl)-[2,2′-bipyrimidin]-4-yl)-2-methylbenzo[d]thiazole;
cis-5-(2-(4-Fluoro-3-methoxyphenyl)cyclopropyl)-4-phenyl-2,2′-bipyrimidine;
cis-5-(2-(4-fluoro-3-methoxyphenyl)cyclopropyl)-4-(piperidin-1-yl)-2,2′-bipyrimidine;
cis-5-(2-(4-fluoro-3,5-dimethoxyphenyl)cyclopropyl)-2,2′-bipyrimidine;
cis-5-(2-(3-chloro-4-fluoro-5-methoxyphenyl)cyclopropyl)-2,2′-bipyrimidine;
cis-5-(2-(4-fluoro-3-methoxy-5-methylphenyl)cyclopropyl)-2,2′-bipyrimidine;
cis-5-(2-(3-methoxy-4-(trifluoromethoxy)phenyl)cyclopropyl)-2,2′-bipyrimidine;
cis-5-(2-(3-methoxy-4-(trifluoromethyl)phenyl)cyclopropyl)-2,2′-bipyrimidine;
cis-5-(2-(5-chloro-4-fluoro-2-(3-methoxypropoxy)phenyl)cyclopropyl)-2,2′-bipyrimidine;
cis-5-(2-(5-chloro-4-fluoro-2-(2-methoxyethoxy)phenyl)cyclopropyl)-2,2′-bipyrimidine;
cis-5-(2-(5-chloro-4-methoxy-[1,1′-biphenyl]-2-yl)cyclopropyl)-2,2′-bipyrimidine;
cis-5-(2-(3,4,5-trifluorophenyl)cyclopropyl)-2,2′-bipyrimidine;
cis-5-(2-(3-methoxy-5-(trifluoromethyl)phenyl)cyclopropyl)-2,2′-bipyrimidine;
cis-5-(2-(3-methoxy-5-(trifluoromethoxy)phenyl)cyclopropyl)-2,2′-bipyrimidine;
cis-5-(2-(4-fluoro-3-methoxy-5-(trifluoromethyl)phenyl)cyclopropyl)-2,2′-bipyrimidine;
cis-5-(2-(naphthalen-1-yl)cyclopropyl)-2,2′-bipyrimidine;
cis-5-(2-(naphthalen-2-yl)cyclopropyl)-2,2′-bipyrimidine;
cis-5-(2-(4-fluoronaphthalen-1-yl)cyclopropyl)-2,2′-bipyrimidine;
cis-5-(2-(4-fluoronaphthalen-2-yl)cyclopropyl)-2,2′-bipyrimidine;
cis-3-(2-([2,2′-bipyrimidin]-5-yl)cyclopropyl)imidazo[1,2-a]pyridine;
cis-5-(2-([2,2′-bipyrimidin]-5-yl)cyclopropyl)quinoline;
cis-5-(2-([2,2′-bipyrimidin]-5-yl)cyclopropyl)-8-fluoroquinoline;
cis-5-(2-([2,2′-bipyrimidin]-5-yl)cyclopropyl)-8-methoxyquinoline;
cis-5-(2-(3,4-difluoro-5-methoxyphenyl)cyclopropyl)-2-(pyridin-2-yl)pyrimidine;
cis-5-(2-(4-fluoro-3,5-dimethoxyphenyl)cyclopropyl)-2-(pyridin-2-yl)pyrimidine;
cis-2-(pyridin-2-yl)-5-(2-(3,4,5-trimethoxyphenyl)cyclopropyl)pyrimidine;
cis-5-(2-(3,4-difluoro-5-methoxyphenyl)cyclopropyl)-2,4′-bipyrimidine;
cis-5-(2-(3,4-difluoro-5-methoxyphenyl)cyclopropyl)-2-(pyrazin-2-yl)pyrimidine;
cis-5-(2-(3,4-difluoro-5-methoxyphenyl)cyclopropyl)-2-(3-fluoropyridin-2-yl)pyrimidine;
cis-5-(2-(3,4-difluoro-5-methoxyphenyl)cyclopropyl)-2-(3-methoxypyridin-2-yl)pyrimidine;
cis-5-(2-(3,4-difluoro-5-(1H-imidazol-1-yl)phenyl)cyclopropyl)-2,2′-bipyrimidine;
cis-5-(2-(5,6-dimethoxypyridin-3-yl)cyclopropyl)-2,2′-bipyrimidine;
cis-5-(2-(3-(difluoromethoxy)-4-fluorophenyl)cyclopropyl)-2,2′-bipyrimidine;
cis-5-(2-(4-fluoro-3-(4-methylpiperazin-1-yl)phenyl)cyclopropyl)-2,2′-bipyrimidine;
cis-5-(2-(2,4-difluoro-3-methoxyphenyl)cyclopropyl)-2,2′-bipyrimidine;
cis-3-(2-(4-fluoro-3-methoxyphenyl)cyclopropyl)-6-(pyrimidin-2-yl)pyridazine;
cis-4-(2-(4-fluoro-3-methoxyphenyl)cyclopropyl)-1-(pyrimidin-2-yl)isoquinoline;
cis-5-(2-(4-fluoro-3-(piperidin-1-yl)phenyl)cyclopropyl)-2,2′-bipyrimidine;
cis-1-(5-(2-([2,2′-bipyrimidin]-5-yl)cyclopropyl)-2-fluorophenyl)pyrrolidin-2-one;
cis-5-(2-(4-chloro-3-(pyrrolidin-1-yl)phenyl)cyclopropyl)-2,2′-bipyrimidine;
cis-5-((2-([2,2′-bipyrimidin]-5-yl)cyclopropyl)-2-chloro-N-methylbenzamide;
cis-5-(2-([2,2′-bipyrimidin]-5-yl)cyclopropyl)-2-chloro-N,N-dimethylbenzamide;
cis-N-(5-(2-([2,2′-bipyrimidin]-5-yl)cyclopropyl)-2-chlorophenyl)acetamide;
cis-5-(2-([2,2′-bipyrimidin]-5-yl)cyclopropyl)-N-cyclopentyl-2,3-difluoroaniline;
cis-6-(5-(2-([2,2′-bipyrimidin]-5-yl)cyclopropyl)-2,3-difluorophenyl)-2-oxa-6-azaspiro[3.3]heptane;
cis-5-(2-(3,4-difluoro-5-(3-isopropoxyazetidin-1-yl)phenyl)cyclopropyl)-2,2′-bipyrimidine;
cis-5-(2-(3-(3-(tert-butyl sulfonyl)azetidin-1-yl)-4,5-difluorophenyl)cyclopropyl)-2,2′-bipyrimidine;
cis-5-(2-(3,4-difluoro-5-(3-(2-methoxyethoxy)azetidin-1-yl)phenyl)cyclopropyl)-2,2′-bipyrimidine;
cis-5-(2-(3-(3-(3,4-difluoro-5-methoxyphenyl)azetidin-1-yl)-4,5-difluorophenyl)cyclopropyl)-2,2′-bipyrimidine;
cis-5-(2-(3-(3-(3,4-difluorophenyl)azetidin-1-yl)-4,5-difluorophenyl)cyclopropyl)-2,2′-bipyrimidine;
cis-5-(2-(3,4-difluoro-5-(3-(4-fluoro-3-methoxyphenyl)azetidin-1-yl)phenyl)cyclopropyl)-2,2′-bipyrimidine;
cis-5-(2-(3-(3-(3,4-dimethoxyphenyl)azetidin-1-yl)-4,5-difluorophenyl)cyclopropyl)-2,2′-bipyrimidine;
cis-5-(2-(3,4-difluoro-5-((1-methyl-1H-1,2,4-triazol-3-yl)methoxy)phenyl)cyclopropyl)-2,2′-bipyrimidine;
cis-2-((5-(2-([2,2′-bipyrimidin]-5-yl)cyclopropyl)-2,3-difluorophenoxy)methyl)thiazole;
cis-5-(2-(3,4-difluoro-5-((1-methyl-1H-pyrazol-3-yl)methoxy)phenyl)cyclopropyl)-2,2′-bipyrimidine;
cis-5-(2-(3-(3,3-dimethylpyrrolidin-1-yl)-4,5-difluorophenyl)cyclopropyl)-2,2′-bipyrimidine;
cis-6-(5-(2-([2,2′-bipyrimidin]-5-yl)cyclopropyl)-2,3-difluorophenyl)-2-oxa-6-azaspiro[3.4]octane;
cis-14(3S)-3-((5-(2-([2,2′-bipyrimidin]-5-yl)cyclopropyl)-2,3-difluorophenyl)amino)pyrrolidin-1-yl)ethenone;
cis-1-((3R)-3-((5-(2-([2,2′-bipyrimidin]-5-yl)cyclopropyl)-2,3-difluorophenyl) amino) pyrrolidin-1-yl)ethenone;
cis-(3S)-N-(5-(2-([2,2′-bipyrimidin]-5-yl)cyclopropyl)-2,3-difluorophenyl)-1-methylpyrrolidin-3-amine;
cis-(3R)-N-(5-(2-([2,2′-bipyrimidin]-5-yl)cyclopropyl)-2,3-difluorophenyl)-1-methylpyrrolidin-3-amine;
cis-5-(2-(3,4-difluoro-5-(4-methyl-1H-pyrazol-1-yl)phenyl)cyclopropyl)-2,2′-bipyrimidine;
cis-N-(5-(2-([2,2′-bipyrimidin]-5-yl)cyclopropyl)-2,3-difluorophenyl)-N-methyloxetan-3-amine;
cis-(1-(5-(2-([2,2′-bipyrimidin]-5-yl)cyclopropyl)-2,3-difluorophenyl)-1H-pyrazol-4-yl)methanol;
cis-5-(2-(3-((3R,4 S)-3,4-dimethoxypyrrolidin-1-yl)-4,5-difluorophenyl)cyclopropyl)-2,2′-bipyrimidine;
cis-5-(2-(3,4-difluoro-5-(4-(methoxymethyl)-1H-pyrazol-1-yl)phenyl)cyclopropyl)-2,2′-bipyrimidine;
cis-1-(5-(2-([2,2′-bipyrimidin]-5-yl)cyclopropyl)-2,3-difluorophenyl)-5,6-dimethoxy-1H-benzo[d]imidazole;
cis-2-(5-(2-([2,2′-bipyrimidin]-5-yl)cyclopropyl)-2,3-difluorophenyl)-2-azaspiro[3.3]heptan-6-ol;
cis-(3R,4R)-1-(5-(2-([2,2′-bipyrimidin]-5-yl)cyclopropyl)-2,3-difluorophenyl)pyrrolidine-3,4-diol;
cis-4-(3-(5-(2-([2,2′-bipyrimidin]-5-yl)cyclopropyl)-2,3-difluorophenoxy)propyl)morpholine;
cis-4-(3-(4-(2-([2,2′-bipyrimidin]-5-yl)cyclopropyl)-2,6-difluorophenoxy)propyl)morpholine;
cis-5-(2-(4-((2-oxaspiro[3.3]heptan-6-yl)oxy)-3,5-difluorophenyl)cyclopropyl)-2,2′-bipyrimidine;
cis-5-(2-(3-((2-oxaspiro[3.3]heptan-6-yl)oxy)-4,5-difluorophenyl)cyclopropyl)-2,2′-bipyrimidine;
cis-2-((5-(2-([2,2′-bipyrimidin]-5-yl)cyclopropyl)-2,3-difluorophenyl)(methyl)amino)ethan-1-ol;
cis-5-(2-(3-(cyclopentyloxy)-4,5-difluorophenyl)cyclopropyl)-2,2′-bipyrimidine;
cis-5-(2-(3,4-difluoro-5-((R)-tetrahydrofuran-3-yl)oxy)phenyl)cyclopropyl)-2,2′-bipyrimidine;
cis-5-(2-(3,4-difluoro-5-((S)-tetrahydrofuran-3-yl)oxy)phenyl)cyclopropyl)-2,2′-bipyrimidine;
cis-5-(2-(3,4-difluoro-5-(4-methoxy-1H-pyrazol-1-yl)phenyl)cyclopropyl)-2,2′-bipyrimidine;
cis-5-(2-(3,4-difluoro-5-(1H-pyrazol-1-yl)phenyl)cyclopropyl)-2,2′-bipyrimidine;
cis-5-(2-(3,4-difluoro-5-(3-phenylazetidin-1-yl)phenyl)cyclopropyl)-2,2′-bipyrimidine;
cis-5-(2-(3-(3,4-difluoro-1H-pyrrol-1-yl)-4,5-difluorophenyl)cyclopropyl)-2,2′-bipyrimidine;
cis-1-(5-(2-([2,2′-bipyrimidin]-5-yl)cyclopropyl)-2,3-difluorophenyl)-1H-pyrazol-4-ol;
cis-5-(2-(3,4-difluoro-5-(4-methyl-1H-imidazol-1-yl)phenyl)cyclopropyl)-2,2′-bipyrimidine;
cis-ethyl 2-([2,2′-bipyrimidin]-5-yl)-3-(3,4-difluoro-5-methoxyphenyl)cyclopropane-1-carboxylate;
cis-4-(2-([2,2′-bipyrimidin]-5-yl)cyclopropyl)-6,7-difluoro-1-(3-methoxypropyl)-1H-indazole;
cis-6-(2-([2,2′-bipyrimidin]-5-yl)cyclopropyl)-4-fluoro-2-(3-methoxypropyl)-2H-indazole;
cis-2-(2-([2,2′-bipyrimidin]-5-yl)cyclopropyl)-4,6-difluorobenzo[d]thiazole;
cis-6-(2-([2,2′-bipyrimidin]-5-yl)cyclopropyl)-4-fluoro-1-isopropyl-2-methyl-1H-benzo[d]imidazole;
cis-6-(2-([2,2′-bipyrimidin]-5-yl)cyclopropyl)-4-fluoro-2-methylbenzo[d]thiazole;
cis-5-(2-([2,2′-bipyrimidin]-5-yl)cyclopropyl)-7-fluoro-1-(3-methoxypropyl)-1H-benzo[d]imidazole;
cis-6-(2-([2,2′-bipyrimidin]-5-yl)cyclopropyl)-4-fluoro-1-(3-methoxypropyl)-1H-benzo[d]imidazole;
cis-6-(2-([2,2′-bipyrimidin]-5-yl)cyclopropyl)-4-fluoro-1-(3-methoxypropyl)-1H-indazole;
cis-4-(2-([2,2′-bipyrimidin]-5-yl)cyclopropyl)-7-fluoro-1-(3-methoxypropyl)-1H-indazole;
cis-4-(2-([2,2′-bipyrimidin]-5-yl)cyclopropyl)-7-fluoro-2-(3-methoxypropyl)-2H-indazole;
cis-5-(2-([2,2′-bipyrimidin]-5-yl)cyclopropyl)-2-methylbenzo[d]thiazole.

The compounds of the disclosure may possess one or more stereocenters, and each stereocenter may exist independently in either the (R) or (S) configuration. In certain embodiments, compounds described herein are present in optically active or racemic forms. The compounds described herein encompass racemic, optically active, regioisomeric and stereoisomeric forms, or combinations thereof that possess the therapeutically useful properties described herein. Preparation of optically active forms is achieved in any suitable manner, including, by way of non-limiting example, by resolution of the racemic form with recrystallization techniques, synthesis from optically active starting materials, chiral synthesis, or chromatographic separation using a chiral stationary phase. A compound illustrated herein by the racemic formula further represents either of the two enantiomers or any mixtures thereof, or in the case where two or more chiral centers are present, all diastereomers or any mixtures thereof.

In certain embodiments, the compounds of the disclosure exist as tautomers. All tautomers are included within the scope of the compounds recited herein.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S.

In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

In all of the embodiments provided herein, examples of suitable optional substituents are not intended to limit the scope of the claimed disclosure. The compounds of the disclosure may contain any of the substituents, or combinations of substituents, provided herein.

### Salts

The compounds described herein may form salts with acids or bases, and such salts are included in the present disclosure. The term “salts” embraces addition salts of free acids or bases that are useful within the methods of the disclosure. The term “pharmaceutically acceptable salt” refers to salts that possess toxicity profiles within a range that affords utility in pharmaceutical applications. In certain embodiments, the salts are pharmaceutically acceptable salts. Pharmaceutically unacceptable salts may nonetheless possess properties such as high crystallinity, which have utility in the practice of the present disclosure, such as for example utility in process of synthesis, purification or formulation of compounds useful within the methods of the disclosure.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, β-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the disclosure.

Suitable pharmaceutically acceptable base addition salts of compounds of the disclosure include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N-dibenzylethylene-diamine, chloroprocaine, choline, diethanolamine, ethylenediamine, meglumine (or N-methylglucamine) and procaine. All of these salts may be prepared from the corresponding compound by reacting, for example, the appropriate acid or base with the compound.

Combination Therapies

In one aspect, the compounds of the disclosure are useful within the methods of the disclosure in combination with one or more additional agents useful for treating HBV and/or HDV infections. These additional agents may comprise compounds or compositions identified herein, or compounds (e.g., commercially available compounds) known to treat, prevent, or reduce the symptoms of HBV and/or HDV infections.

Non-limiting examples of one or more additional agents useful for treating HBV and/or HDV infections include: (a) reverse transcriptase inhibitors; (b) capsid inhibitors; (c) cccDNA formation inhibitors; (d) RNA destabilizers; (e) oligomeric nucleotides targeted against the HBV genome; (f) immunostimulators, such as checkpoint inhibitors (e.g., PD-L1 inhibitors); and (g) GalNAc-siRNA conjugates targeted against an HBV gene transcript.

(a) Reverse Transcriptase Inhibitors

In certain embodiments, the reverse transcriptase inhibitor is a reverse-transcriptase inhibitor (NARTI or NRTI). In other embodiments, the reverse transcriptase inhibitor is a nucleotide analog reverse-transcriptase inhibitor (NtARTI or NtRTI).

Reported reverse transcriptase inhibitors include, but are not limited to, entecavir, clevudine, telbivudine, lamivudine, adefovir, and tenofovir, tenofovir disoproxil, tenofovir alafenamide, adefovir dipovoxil, (1R,2R,3R,5R)-3-(6-amino-9H-9-purinyl)-2-fluoro-5-(hydroxymethyl)-4-methylenecyclopentan-1-ol (described in U.S. Pat. No. 8,816,074, incorporated herein in its entirety by reference), emtricitabine, abacavir, elvucitabine, ganciclovir, lobucavir, famciclovir, penciclovir, and amdoxovir.

Reported reverse transcriptase inhibitors further include, but are not limited to, entecavir, lamivudine, and (1R,2R,3R,5R)-3-(6-amino-9H-9-purinyl)-2-fluoro-5-(hydroxymethyl)-4-methylenecyclopentan-1-ol.

Reported reverse transcriptase inhibitors further include, but are not limited to, a covalently bound phosphoramidate or phosphonamidate moiety of the above-mentioned reverse transcriptase inhibitors, or as described in for example U.S. Pat. No. 8,816,074, US Patent Application Publications No. US 2011/0245484 A1, and US 2008/0286230A1, all of which incorporated herein in their entireties by reference.

Reported reverse transcriptase inhibitors further include, but are not limited to, nucleotide analogs that comprise a phosphoramidate moiety, such as, for example, methyl ((((1R,3R,4R,5R)-3-(6-amino-9H-purin-9-yl)-4-fluoro-5-hydroxy-2-methylenecyclopentyl) methoxy)(phenoxy) phosphoryl)-(D or L)-alaninate and methyl ((((1R2R,3R,4R)-3-fluoro-2-hydroxy-5-methylene-4-(6-oxo-1,6-dihydro-9H-purin-9-yl)cyclopentyl)methoxy)(phenoxy) phosphoryl)-(D or L)-alaninate. Also included are the individual diastereomers thereof, which include, for example, methyl ((R)-(((1R,3R,4R,5R)-3-(6-amino-9H-purin-9-yl)-4-fluoro-5-hydroxy-2-methylenecyclopentyl)methoxy)(phenoxy)phosphoryl)-(D or L)-alaninate and methyl ((S)-(((1R,3R,4R,5R)-3-(6-amino-9H-purin-9-yl)-4-fluoro-5-hydroxy-2-methylenecyclopentyl) methoxy)(phenoxy)phosphoryl)-(D or L)-alaninate.

Reported reverse transcriptase inhibitors further include, but are not limited to, compounds comprising a phosphonamidate moiety, such as, for example, tenofovir alafenamide, as well as those described in U.S. Patent Application Publication No. US 2008/0286230 A1, incorporated herein in its entirety by reference. Methods for preparing stereoselective phosphoramidate or phosphonamidate containing actives are described in, for example, U.S. Pat. No. 8,816,074, as well as U.S. Patent Application Publications No. US 2011/0245484 A1 and US 2008/0286230 A1, all of which incorporated herein in their entireties by reference.

(b) Capsid Inhibitors

As described herein, the term “capsid inhibitor” includes compounds that are capable of inhibiting the expression and/or function of a capsid protein either directly or indirectly. For example, a capsid inhibitor may include, but is not limited to, any compound that inhibits capsid assembly, induces formation of non-capsid polymers, promotes excess capsid assembly or misdirected capsid assembly, affects capsid stabilization, and/or inhibits encapsidation of RNA (pgRNA). Capsid inhibitors also include any compound that inhibits capsid function in a downstream event(s) within the replication process (e.g., viral DNA synthesis, transport of relaxed circular DNA (rcDNA) into the nucleus, covalently closed circular DNA (cccDNA) formation, virus maturation, budding and/or release, and the like). For example, in certain embodiments, the inhibitor detectably inhibits the expression level or biological activity of the capsid protein as measured, e.g., using an assay described herein. In certain embodiments, the inhibitor inhibits the level of rcDNA and downstream products of viral life cycle by at least 5%, at least 10%, at least 20%, at least 50%, at least 75%, or at least 90%.

Reported capsid inhibitors include, but are not limited to, compounds described in International Patent Applications Publication Nos WO 2013006394, WO 2014106019, and W02014089296, all of which incorporated herein in their entireties by reference.

Reported capsid inhibitors also include, but are not limited to, the following compounds and pharmaceutically acceptable salts and/or solvates thereof: Bay-41-4109 (see Int'l Patent Application Publication No. WO 2013144129), AT-61 (see Int'l Patent Application Publication No. WO 1998033501; and King, et al., 1998, Antimicrob. Agents Chemother. 42(12):3179-3186), DVR-01 and DVR-23 (see Int'l Patent Application Publication No. WO 2013006394; and Campagna, et al., 2013, J. Virol. 87(12):6931, all of which incorporated herein in their entireties by reference.

In addition, reported capsid inhibitors include, but are not limited to, those generally and specifically described in U.S. Patent Application Publication Nos. US 2015/0225355, US 2015/0132258, US 2016/0083383, US 2016/0052921, US 2019/0225593, and Int'l Patent Application Publication Nos. WO 2013096744, WO 2014165128, WO 2014033170, WO 2014033167, WO 2014033176, WO 2014131847, WO 2014161888, WO 2014184350, WO 2014184365, WO 2015059212, WO 2015011281, WO 2015118057, WO 2015109130, WO 2015073774, WO 2015180631, WO 2015138895, WO 2016089990, WO 2017015451, WO 2016183266, WO 2017011552, WO 2017048950, W02017048954, WO 2017048962, WO 2017064156, WO 2018052967, WO 2018172852, WO 2020023710 and are incorporated herein in their entirety by reference.

(c) cccDNA Formation Inhibitors

Covalently closed circular DNA (cccDNA) is generated in the cell nucleus from viral rcDNA and serves as the transcription template for viral mRNAs. As described herein, the term “cccDNA formation inhibitor” includes compounds that are capable of inhibiting the formation and/or stability of cccDNA either directly or indirectly. For example, a cccDNA formation inhibitor may include, but is not limited to, any compound that inhibits capsid disassembly, rcDNA entry into the nucleus, and/or the conversion of rcDNA into cccDNA. For example, in certain embodiments, the inhibitor detectably inhibits the formation and/or stability of the cccDNA as measured, e.g., using an assay described herein. In certain embodiments, the inhibitor inhibits the formation and/or stability of cccDNA by at least 5%, at least 10%, at least 20%, at least 50%, at least 75%, or at least 90%.

Reported cccDNA formation inhibitors include, but are not limited to, compounds described in Int'l Patent Application Publication No. WO 2013130703, and are incorporated herein in their entirety by reference.

In addition, reported cccDNA formation inhibitors include, but are not limited to, those generally and specifically described in U.S. Patent Application Publication No. US 2015/0038515 A1, and are incorporated herein in their entirety by reference.

(d) RNA Destabilizer

As used herein, the term “RNA destabilizer” refers to a molecule, or a salt or solvate thereof, that reduces the total amount of HBV RNA in mammalian cell culture or in a live human subject. In a non-limiting example, an RNA destabilizer reduces the amount of the RNA transcript(s) encoding one or more of the following HBV proteins: surface antigen, core protein, RNA polymerase, and e antigen. In certain embodiments, the RNA destabilizer reduces the total amount of HBV RNA in mammalian cell culture or in a live human subject by at least 5%, at least 10%, at least 20%, at least 50%, at least 75%, or at least 90%. Reported RNA destabilizers include compounds described in U.S. Pat. No.

8,921,381, as well as compounds described in U.S. Patent Application Publication Nos. US 2015/0087659 and US 2013/0303552, all of which are incorporated herein in their entireties by reference.

In addition, reported RNA destabilizers include, but are not limited to, those generally and specifically described in Int'l Patent Application Publication Nos. WO 2015113990, WO 2015173164, US 2016/0122344, WO 2016107832, WO 2016023877, WO 2016128335, WO 2016177655, WO 2016071215, WO 2017013046, WO 2017016921, WO 2017016960, WO 2017017042, WO 2017017043, WO 2017102648, WO 2017108630, WO 2017114812, WO 2017140821, WO 2018085619, and are incorporated herein in their entirety by reference.

(e) Oligomeric Nucleotides Targeted Against the HBV Genome

Reported oligomeric nucleotides targeted against the HBV genome include, but are not limited to, Arrowhead-ARC-520 (see U.S. Pat. No. 8,809,293; and Wooddell et al., 2013, Molecular Therapy 21(5):973-985, all of which incorporated herein in their entireties by reference).

In certain embodiments, the oligomeric nucleotides can be designed to target one or more genes and/or transcripts of the HBV genome. Oligomeric nucleotide targeted to the HBV genome also include, but are not limited to, isolated, double stranded, siRNA molecules, that each include a sense strand and an antisense strand that is hybridized to the sense strand. In certain embodiments, the siRNA target one or more genes and/or transcripts of the HBV genome.

(f) Immunostimulators

Checkpoint Inhibitors

As described herein, the term “checkpoint inhibitor” includes any compound that is capable of inhibiting immune checkpoint molecules that are regulators of the immune system (e.g., stimulate or inhibit immune system activity). For example, some checkpoint inhibitors block inhibitory checkpoint molecules, thereby stimulating immune system function, such as stimulation of T cell activity against cancer cells. A non-limiting example of a checkpoint inhibitor is a PD-L1 inhibitor.

As described herein, the term “PD-L1 inhibitor” includes any compound that is capable of inhibiting the expression and/or function of the protein Programmed Death-Ligand 1 (PD-L1) either directly or indirectly. PD-L1, also known as cluster of differentiation 274 (CD274) or B7 homolog 1 (B7-H1), is a type 1 transmembrane protein that plays a major role in suppressing the adaptive arm of immune system during pregnancy, tissue allograft transplants, autoimmune disease, and hepatitis. PD-L1 binds to its receptor, the inhibitory checkpoint molecule PD-1 (which is found on activated T cells, B cells, and myeloid cells) so as to modulate activation or inhibition of the adaptive arm of immune system. In certain embodiments, the PD-L1 inhibitor inhibits the expression and/or function of PD-L1 by at least 5%, at least 10%, at least 20%, at least 50%, at least 75%, or at least 90%.

Reported PD-L1 inhibitors include, but are not limited to, compounds recited in one of the following patent application publications: US 2018/0057455; US 2018/0057486; WO 2017/106634; WO 2018/026971; WO 2018/045142; WO 2018/118848; WO 2018/119221; WO 2018/119236; WO 2018/119266; WO 2018/119286; WO 2018/121560; WO 2019/076343; WO 2019/087214; and are incorporated herein in their entirety by reference.

(g) GalNAc-siRNA Conjugates Targeted Against an HBV Gene Transcript

“GalNAc” is the abbreviation for N-acetylgalactosamine, and “siRNA” is the abbreviation for small interfering RNA. An siRNA that targets an HBV gene transcript is covalently bonded to GalNAc in a GalNAc-siRNA conjugate useful in the practice of the present disclosure. While not wishing to be bound by theory, it is believed that GalNAc binds to asialoglycoprotein receptors on hepatocytes thereby facilitating the targeting of the siRNA to the hepatocytes that are infected with HBV. The siRNA enter the infected hepatocytes and stimulate destruction of HBV gene transcripts by the phenomenon of RNA interference.

Examples of GalNAc-siRNA conjugates useful in the practice of this aspect of the present disclosure are set forth in published international application PCT/CA2017/050447 (PCT Application Publication number WO/2017/177326, published on Oct. 19, 2017) which is hereby incorporated by reference in its entirety.

A synergistic effect may be calculated, for example, using suitable methods such as, for example, the Sigmoid-Emax equation (Holford & Scheiner, 1981, Clin. Pharmacokinet. 6:429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22:27-55). Each equation referred to elsewhere herein may be applied to experimental data to generate a corresponding graph to aid in assessing the effects of the drug combination. The corresponding graphs associated with the equations referred to elsewhere herein are the concentration-effect curve, isobologram curve and combination index curve, respectively.

### Synthesis

The present disclosure further provides methods of preparing compounds of the present disclosure. Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field.

It is appreciated that where typical or preferred process conditions (i.e., reaction temperatures, times, mole ratios of reactants, solvents, pressures, and so forth) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high-performance liquid chromatograpy (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene, et al., Protective Groups in Organic

Synthesis, 2d. Ed. (Wiley & Sons, 1991), the entire disclosure of which is incorporated by reference herein for all purposes.

The reactions or the processes described herein can be carried out in suitable solvents that can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

In the following schemes, compounds with R^{4a}═R^{4b}═H are illustrated in a non-limiting manner. Likewise, compounds wherein R^{4a} and/or R^{4b} is/are not H can be prepared using the synthetic procedures illustrated herein starting with appropriate starting materials.

In certain embodiments, a compound of the disclosure can be prepared, for example, according to the illustrative synthetic methods outlined in Scheme I:

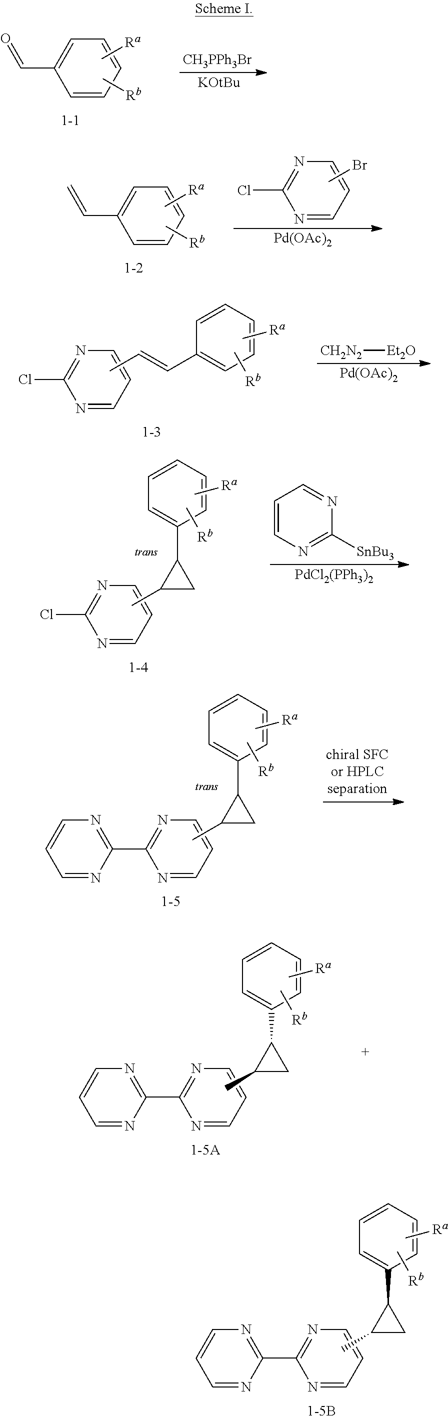

In certain embodiments, a compound of the disclosure can be prepared, for example, according to the illustrative synthetic methods outlined in Scheme II:

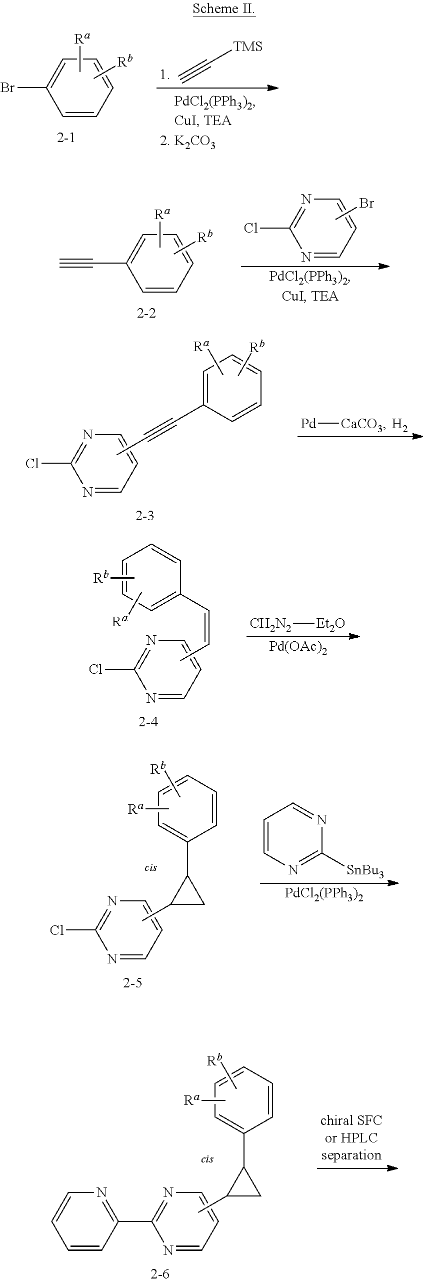

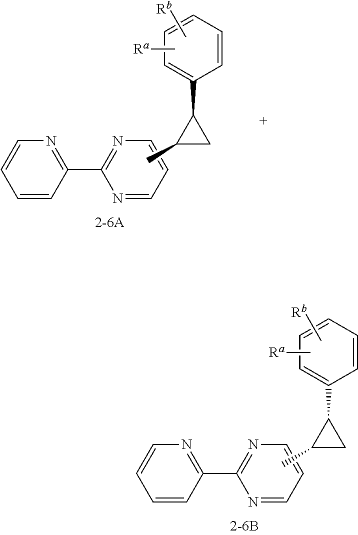

In certain embodiments, a compound of the disclosure can be prepared, for example, according to the illustrative synthetic methods outlined in Scheme III:

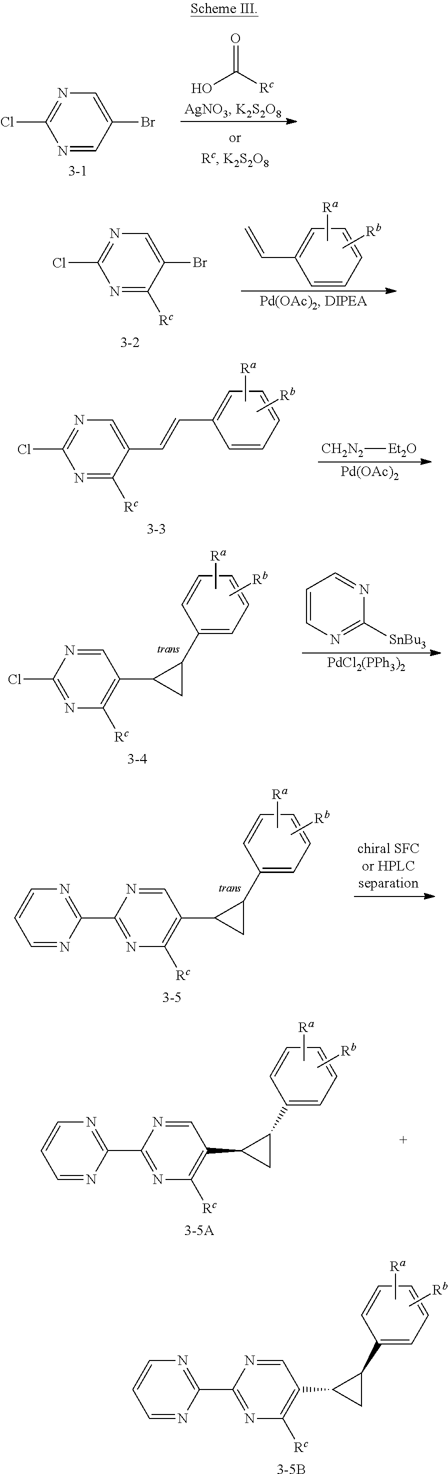

In certain embodiments, a compound of the disclosure can be prepared, for example, according to the illustrative synthetic methods outlined in Scheme IV:

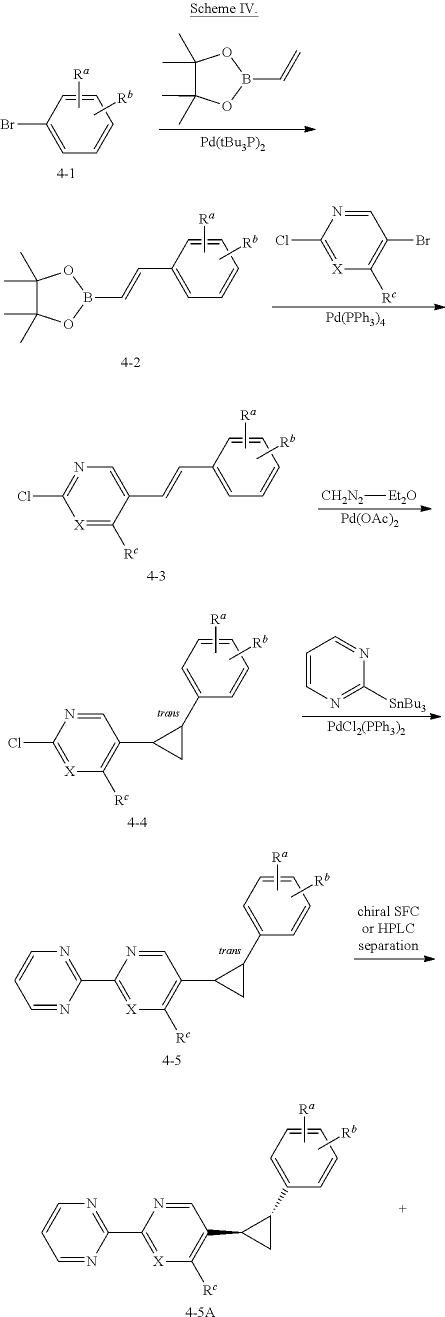

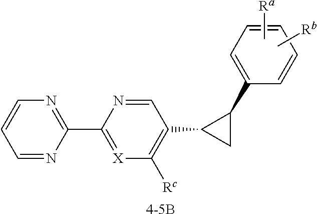

In certain embodiments, a compound of the disclosure can be prepared, for example, according to the illustrative synthetic methods outlined in Scheme V:

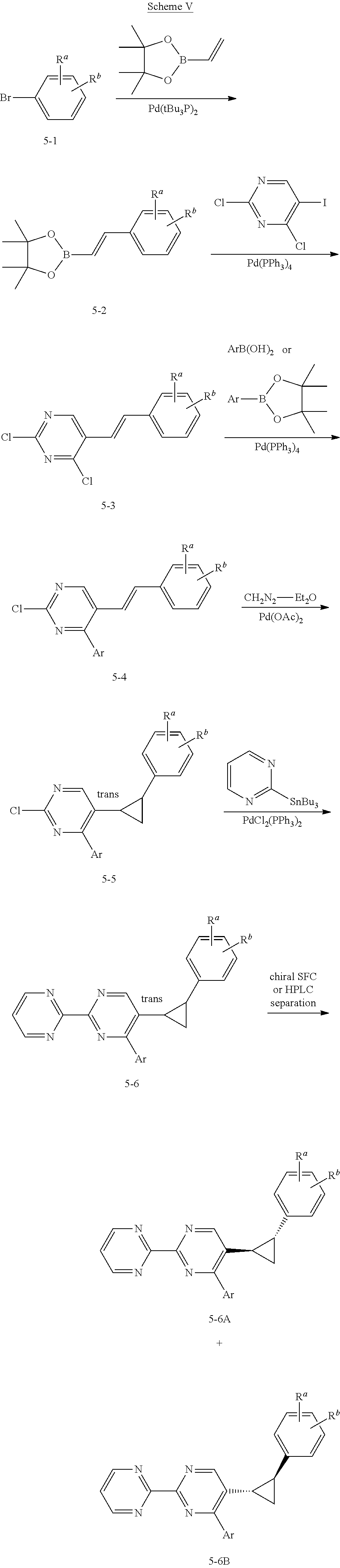

In certain embodiments, a compound of the disclosure can be prepared, for example, according to the illustrative synthetic methods outlined in Scheme VI:

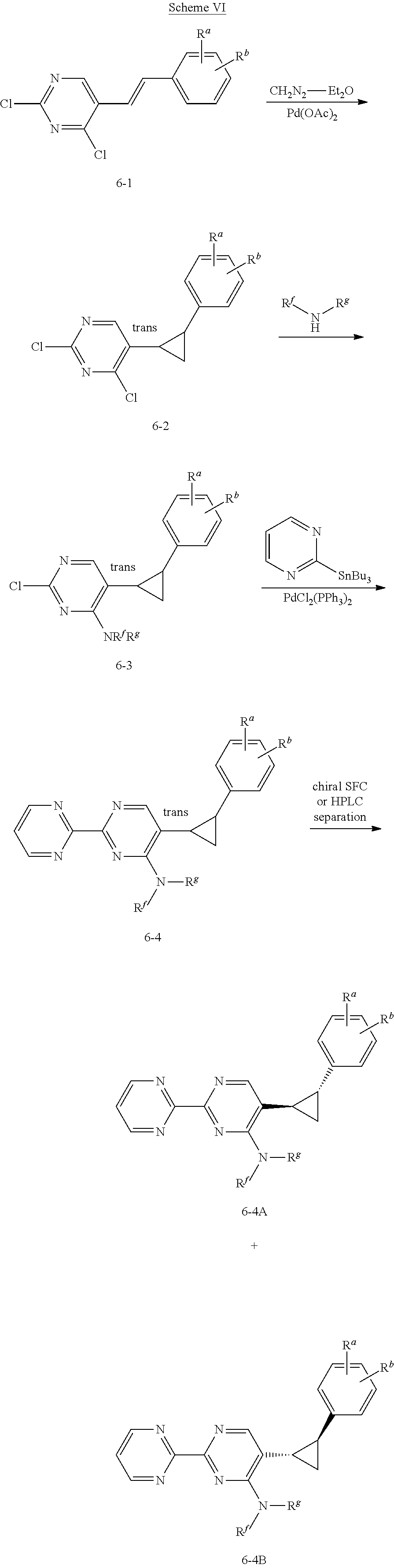

In certain embodiments, a compound of the disclosure can be prepared, for example, according to the illustrative synthetic methods outlined in Scheme VII:

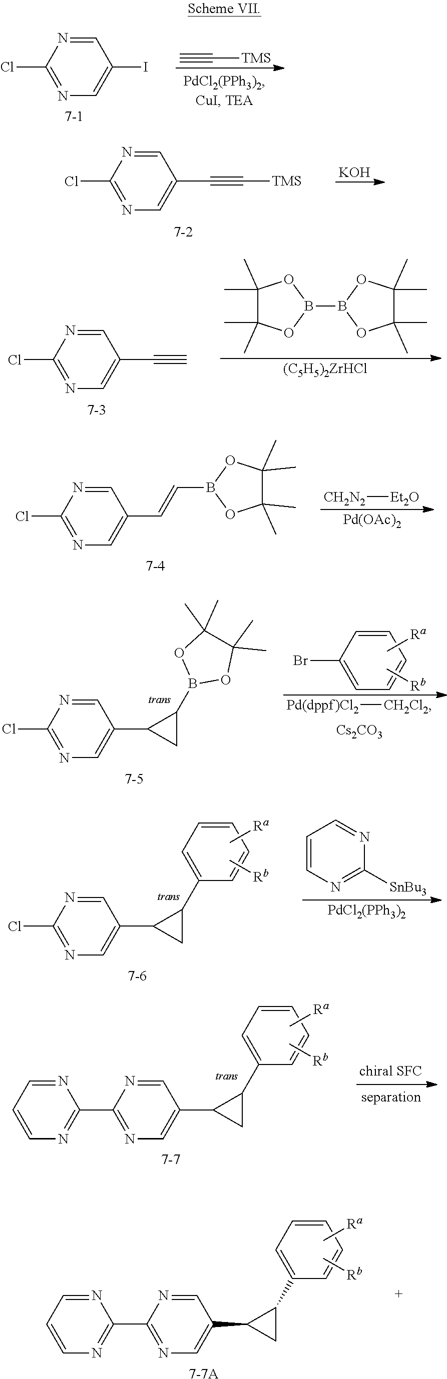

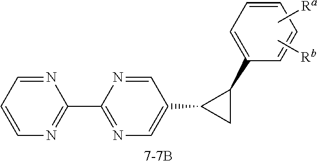

In certain embodiments, a compound of the disclosure can be prepared, for example, according to the illustrative synthetic methods outlined in Scheme VIII:

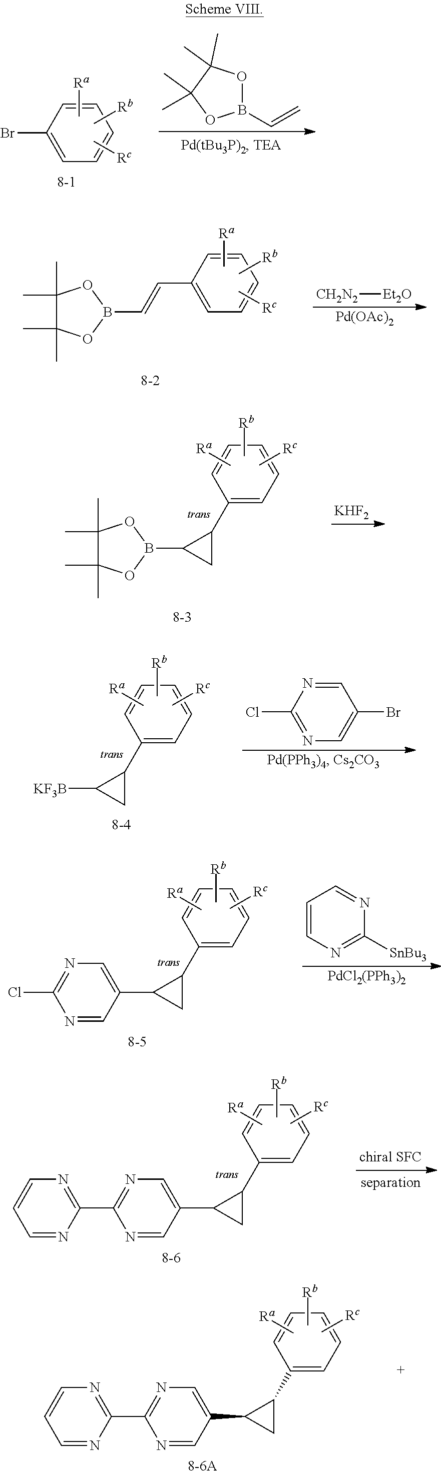

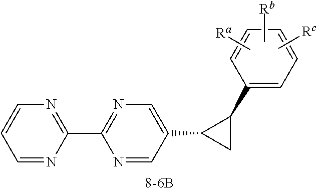

In certain embodiments, a compound of the disclosure can be prepared, for example, according to the illustrative synthetic methods outlined in Scheme IX:

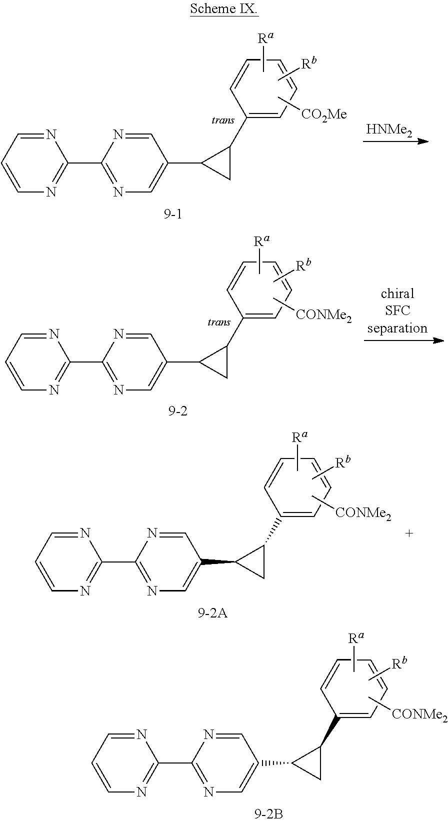

In certain embodiments, a compound of the disclosure can be prepared, for example, according to the illustrative synthetic methods outlined in Scheme X:

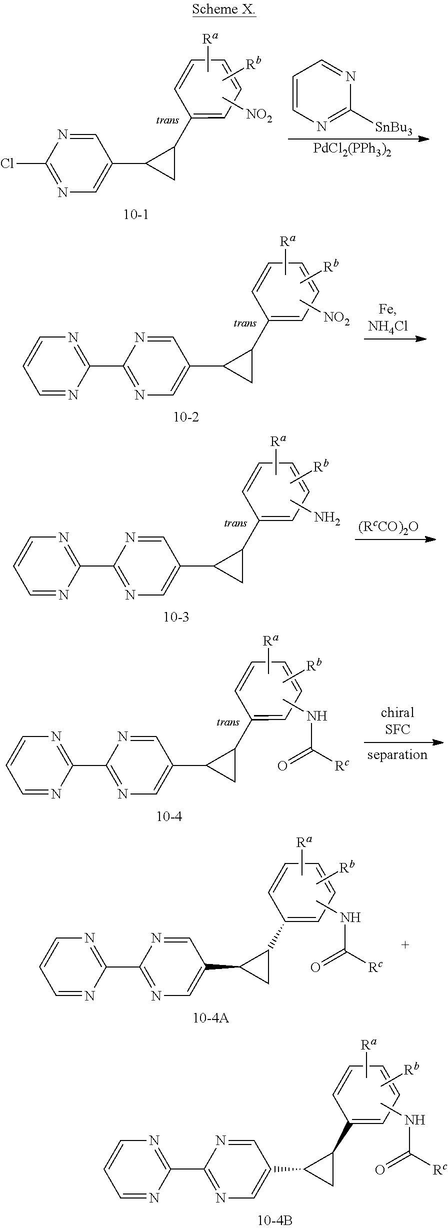

In certain embodiments, a compound of the disclosure can be prepared, for example, according to the illustrative synthetic methods outlined in Scheme XI:

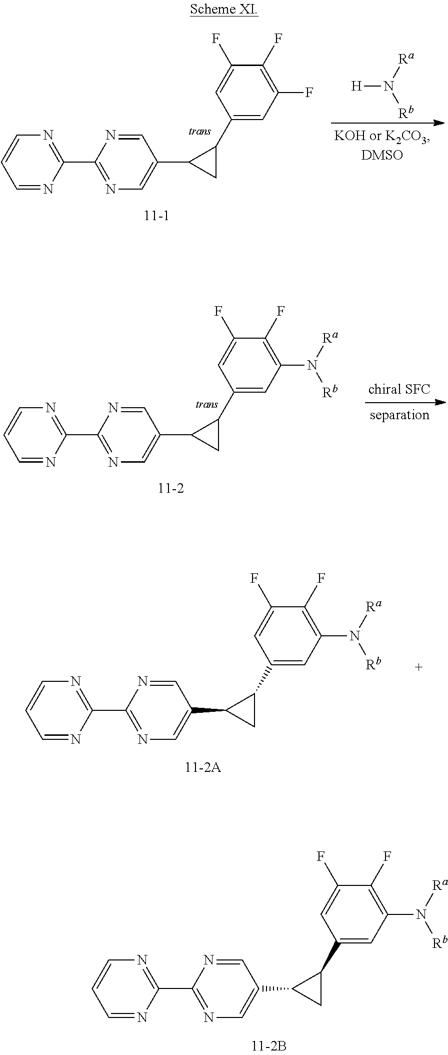

In certain embodiments, a compound of the disclosure can be prepared, for example, according to the illustrative synthetic methods outlined in Scheme XII:

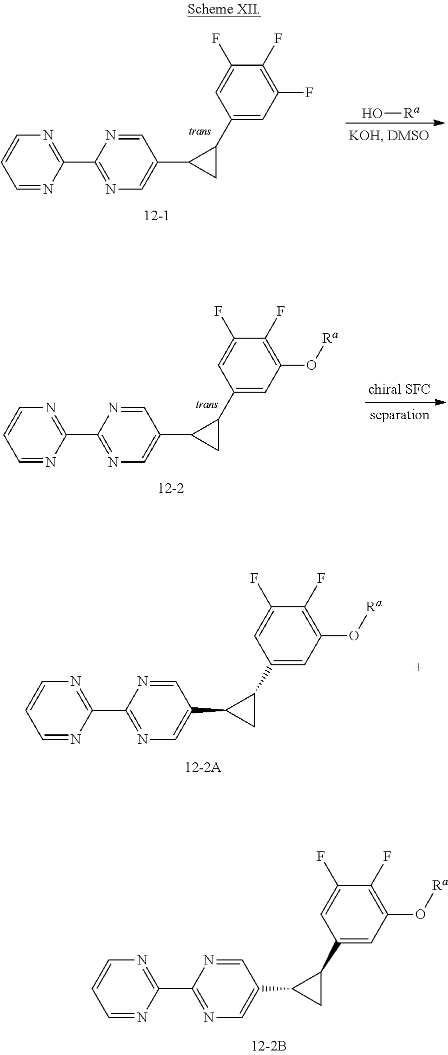

In certain embodiments, a compound of the disclosure can be prepared, for example, according to the illustrative synthetic methods outlined in Scheme XIII:

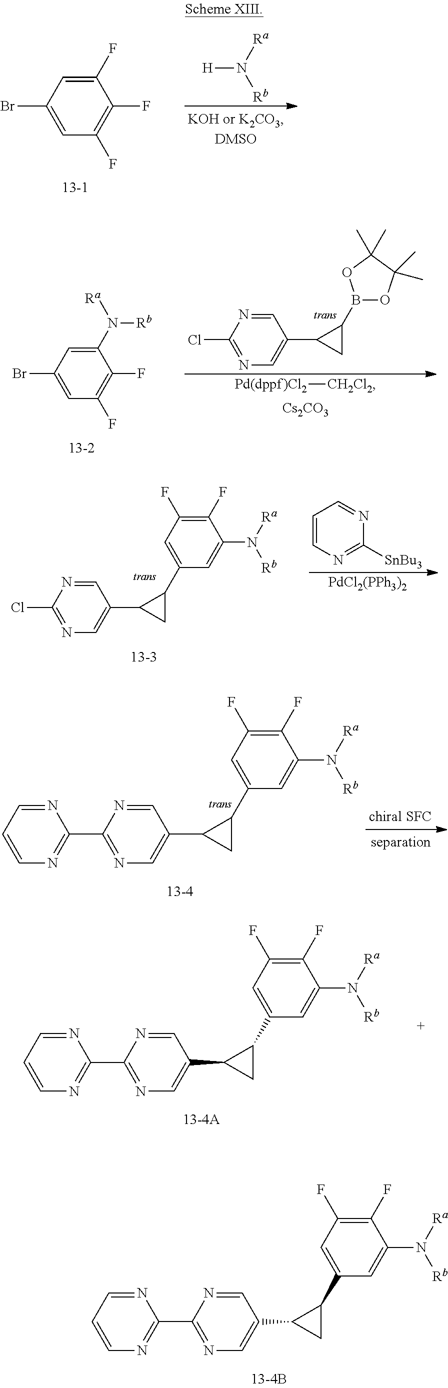

In certain embodiments, a compound of the disclosure can be prepared, for example, according to the illustrative synthetic methods outlined in Scheme XIV:

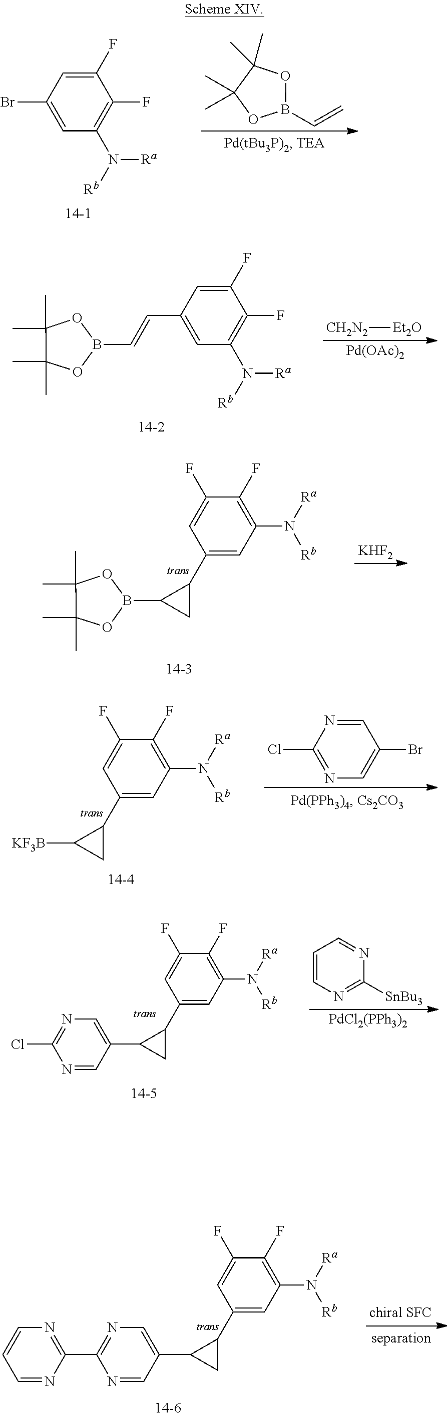

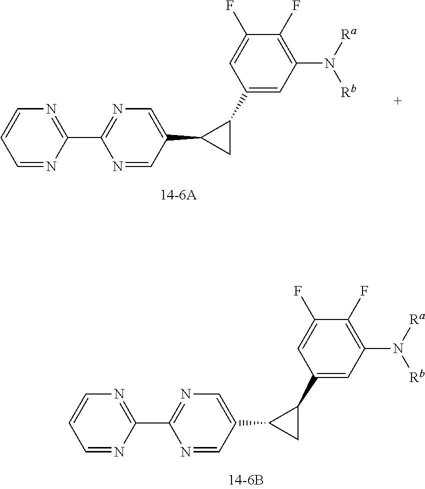

In certain embodiments, a compound of the disclosure can be prepared, for example, according to the illustrative synthetic methods outlined in Scheme XV:

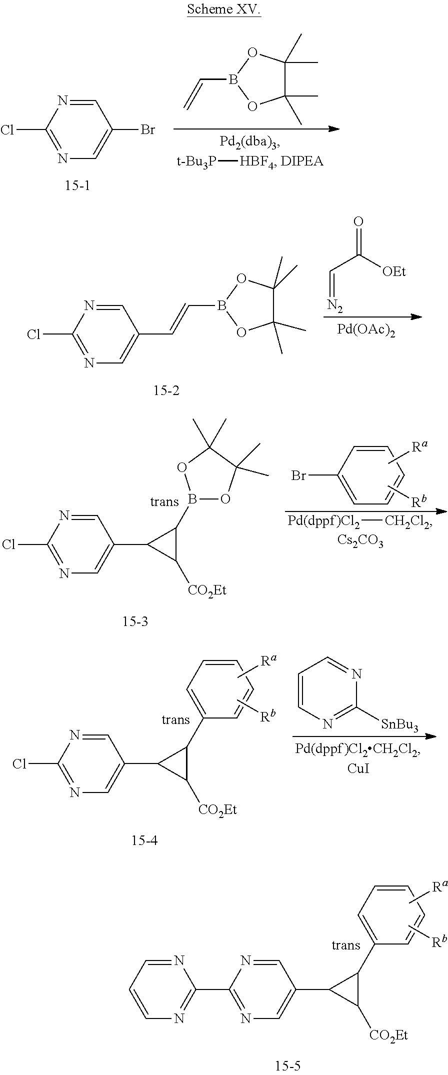

In certain embodiments, a compound of the disclosure can be prepared, for example, according to the illustrative synthetic methods outlined in Scheme XVI:

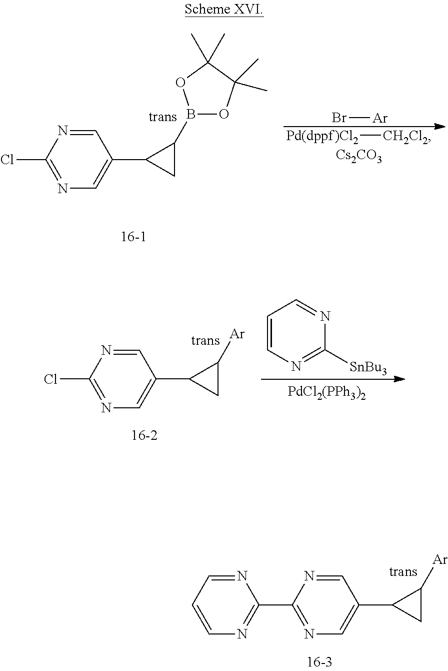

### Methods

The disclosure provides a method of treating or preventing hepatitis virus infection in a subject. In certain embodiments, the virus comprises hepatitis B virus (HBV). In other embodiments, the virus comprises hepatitis D virus (HDV). In yet other embodiments, the virus comprises HBV and HDV. In yet other embodiments, the method comprises administering to the subject in need thereof a therapeutically effective amount of at least one compound of the disclosure. In yet other embodiments, the compound of the disclosure is the only antiviral agent administered to the subject. In yet other embodiments, the at least one compound is administered to the subject in a pharmaceutically acceptable composition. In yet other embodiments, the subject is further administered at least one additional agent useful for treating the hepatitis virus infection. In yet other embodiments, the at least one additional agent comprises at least one selected from the group consisting of reverse transcriptase inhibitors, capsid inhibitors, cccDNA formation inhibitors, RNA destabilizers, oligomeric nucleotides targeted against the HBV genome, immunostimulators, and GalNAc-siRNA conjugates targeted against an HBV gene transcript. In yet other embodiments, the subject is co-administered the at least one compound and the at least one additional agent. In yet other embodiments, the at least one compound and the at least one additional agent are coformulated.

The disclosure further provides a method of inhibiting and/or reducing HBV surface antigen (HBsAg) secretion either directly or indirectly in a subject. The disclosure further provides a method of reducing or minimizing levels of HBsAg in an HBV-infected subject. The disclosure further provides a method of reducing or minimizing levels of HBeAg in an HBV-infected subject. The disclosure further provides a method of reducing or minimizing levels of hepatitis B core protein in an HBV-infected subject. The disclosure further provides a method of reducing or minimizing levels of pg RNA in an HBV-infected subject.

In certain embodiments, the method comprises administering to the subject in need thereof a therapeutically effective amount of at least one compound of the disclosure. In other embodiments, the at least one compound is administered to the subject in a pharmaceutically acceptable composition. In yet other embodiments, the compound of the disclosure is the only antiviral agent administered to the subject. In yet other embodiments, the subject is further administered at least one additional agent useful for treating HBV infection. In yet other embodiments, the at least one additional agent comprises at least one selected from the group consisting of reverse transcriptase inhibitors, capsid inhibitors, cccDNA formation inhibitors, RNA destabilizers, oligomeric nucleotides targeted against the HBV genome, immunostimulators, and GalNAc-siRNA conjugates targeted against an HBV gene transcript. In yet other embodiments, the subject is co-administered the at least one compound and the at least one additional agent. In yet other embodiments, the at least one compound and the at least one additional agent are coformulated.

In certain embodiments, the subject is a subject in need thereof.

In certain embodiments, the subject is a mammal. In other embodiments, the mammal is a human.

### Pharmaceutical Compositions and Formulations

The disclosure provides pharmaceutical compositions comprising at least one compound of the disclosure or a salt or solvate thereof, which are useful to practice methods of the disclosure. Such a pharmaceutical composition may consist of at least one compound of the disclosure or a salt or solvate thereof, in a form suitable for administration to a subject, or the pharmaceutical composition may comprise at least one compound of the disclosure or a salt or solvate thereof, and one or more pharmaceutically acceptable carriers, one or more additional ingredients, or any combinations of these. At least one compound of the disclosure may be present in the pharmaceutical composition in the form of a physiologically acceptable salt, such as in combination with a physiologically acceptable cation or anion, as is well known in the art.

In certain embodiments, the pharmaceutical compositions useful for practicing the method of the disclosure may be administered to deliver a dose of between 1 ng/kg/day and 100 mg/kg/day. In other embodiments, the pharmaceutical compositions useful for practicing the disclosure may be administered to deliver a dose of between 1 ng/kg/day and 1,000 mg/kg/day.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the disclosure may be suitably developed for nasal, inhalational, oral, rectal, vaginal, pleural, peritoneal, parenteral, topical, transdermal, pulmonary, intranasal, buccal, ophthalmic, epidural, intrathecal, intravenous, or another route of administration. A composition useful within the methods of the disclosure may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In certain embodiments, the compositions of the disclosure are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as cattle, pigs, horses, sheep, cats, and dogs.

In certain embodiments, the compositions of the disclosure are formulated using one or more pharmaceutically acceptable excipients or carriers. In certain embodiments, the pharmaceutical compositions of the disclosure comprise a therapeutically effective amount of at least one compound of the disclosure and a pharmaceutically acceptable carrier. Pharmaceutically acceptable carriers, which are useful, include, but are not limited to, glycerol, water, saline, ethanol, recombinant human albumin (e.g., RECOMBUMIN®), solubilized gelatins (e.g., GELOFUSINE®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring, and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the disclosure may comprise a preservative from about 0.005% to 2.0% by total weight of the composition. The preservative is used to prevent spoilage in the case of exposure to contaminants in the environment. Examples of preservatives useful in accordance with the disclosure include but are not limited to those selected from the group consisting of benzyl alcohol, sorbic acid, parabens, imidurea and any combinations thereof. One such preservative is a combination of about 0.5% to 2.0% benzyl alcohol and 0.05-0.5% sorbic acid.

The composition may include an antioxidant and a chelating agent that inhibit the degradation of the compound. Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.01% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non-ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the disclosure may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the disclosure may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, ionic and non-ionic surfactants, and a preservative. Additional excipients, such as fillers and sweetening, flavoring, or coloring agents, may also be included in these formulations.

A pharmaceutical composition of the disclosure may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, such as a mammal, such as a human, may be carried out using known procedures, at dosages and for periods of time effective to treat a disease or disorder contemplated herein. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the disclosure is from about 0.01 mg/kg to 100 mg/kg of body weight/per day. One of ordinary skill in the art would be able to study the relevant factors and make the determination regarding the effective amount of the therapeutic compound without undue experimentation.

The compound may be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder in a patient.

In certain embodiments, the compositions of the disclosure are administered to the patient in dosages that range from one to five times per day or more. In other embodiments, the compositions of the disclosure are administered to the patient in range of dosages that include, but are not limited to, once every day, every two days, every three days to once a week, and once every two weeks. It will be readily apparent to one skilled in the art that the frequency of administration of the various combination compositions of the disclosure will vary from subject to subject depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physician taking all other factors about the patient into account.

Compounds of the disclosure for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of a compound of the disclosure is from about 0.5 μg and about 5,000 mg. In some embodiments, a dose of a compound of the disclosure used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder in a patient.

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

### Administration

Routes of administration of any of the compositions of the disclosure include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic, generally recognized as safe (GRAS) pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation. Hard capsules comprising the active ingredient may be made using a physiologically degradable composition, such as gelatin. The capsules comprise the active ingredient, and may further comprise additional ingredients including, for example, an inert solid diluent such as calcium carbonate, calcium phosphate, or kaolin.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin from animal-derived collagen or from a hypromellose, a modified form of cellulose, and manufactured using optional mixtures of gelatin, water and plasticizers such as sorbitol or glycerol. Such soft capsules comprise the active ingredient, which may be mixed with water or an oil medium such as peanut oil, liquid paraffin, or olive oil.

For oral administration, the compounds of the disclosure may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY® film coating systems available from Colorcon, West Point, Pa. (e.g., OPADRY® OY Type, OYC Type, Organic Enteric OY-P Type, Aqueous Enteric OY-A Type, OY-PM Type and OPADRY® White, 32K18400). It is understood that similar type of film coating or polymeric products from other companies may be used.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface-active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present disclosure also includes a multi-layer tablet comprising a layer providing for the delayed release of one or more compounds useful within the methods of the disclosure, and a further layer providing for the immediate release of one or more compounds useful within the methods of the disclosure. Using a wax/pH-sensitive polymer mix, a gastric insoluble composition may be obtained in which the active ingredient is entrapped, ensuring its delayed release.

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the disclosure which are suitable for oral administration may be prepared, packaged, and sold either in liquid form or in the form of a dry product intended for reconstitution with water or another suitable vehicle prior to use.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the disclosure may contain liposomes. The composition of the liposomes and their use are known in the art (i.e., U.S. Pat. No. 6,323,219).

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In other embodiments, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. For example, it should be present in an amount from about 0.0005% to about 5% of the composition; for example, it should be present in an amount of from about 0.001% to about 1% of the composition. Such compounds may be synthetically-or naturally derived.

### Buccal Administration

A pharmaceutical composition of the disclosure may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, may have an average particle or droplet size in the range from about 0.1 to about 200 nanometers, and may further comprise one or more of the additional ingredients described herein. The examples of formulations described herein are not exhaustive and it is understood that the disclosure includes additional modifications of these and other formulations not described herein, but which are known to those of skill in the art.

### Rectal Administration

A pharmaceutical composition of the disclosure may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Additional Administration Forms

Additional dosage forms of this disclosure include dosage forms as described in U.S. Pat. Nos. 6,340,475, 6,488,962, 6,451,808, 5,972,389, 5,582,837, and 5,007,790. Additional dosage forms of this disclosure also include dosage forms as described in U.S. Patent Applications Nos. 20030147952, 20030104062, 20030104053, 20030044466, 20030039688, and 20020051820. Additional dosage forms of this disclosure also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems:

In certain embodiments, the compositions and/or formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In certain embodiments of the disclosure, the compounds useful within the disclosure are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the disclosure. Accordingly, all values and ranges encompassed by these values and ranges are meant to be encompassed within the scope of the present disclosure. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present disclosure. However, they are in no way a limitation of the teachings or disclosure of the present disclosure as set forth herein.

### EXAMPLES

The disclosure is now described with reference to the following Examples. These Examples are provided for the purpose of illustration only, and the disclosure is not limited to these Examples, but rather encompasses all variations that are evident as a result of the teachings provided herein.

### Materials & Methods

The following procedures can be utilized in preparing and/or testing exemplary compounds of the disclosure.

As described herein, “Enantiomer I” or “Diastereoisomer I” refers to the first enantiomer or diastereoisomer eluded from the chiral column under the specific chiral analytical conditions detailed for examples provided elsewhere herein; and “Enantiomer II” or “Diastereoisomer II” refers to the second enantiomer or diastereoisomer eluded from the chiral column under the specific chiral analytical conditions detailed for examples provided elsewhere herein. Such nomenclature does not imply or impart any particular relative and/or absolute configuration for these compounds.

### Example 1: trans-5-(2-(2-Fluoro-4-methoxyphenyl)cyclopropyl)-2,2′-bipyrimidine

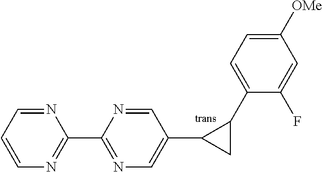 [SMILES: COc1ccc(C2CC2c2cnc(-c3ncccn3)nc2)c(F)c1]

### 2-Fluoro-4-methoxy-1-vinylbenzene

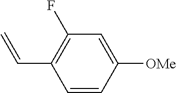

To a cooled solution of methyltriphenylphosphonium bromide (9.3 g, 26 mmol) in THF (20 mL) was added potassium tert-butoxide solution (1.0M in THF, 19.6 mL, 19.6 mmol) at 0° C., and the mixture was stirred for 30 min. To the reaction mixture was added 2-fluoro-4-methoxybenzaldehyde (2.0 g, 12.9 mmol, 1.0 eq.) at 0° C., and the mixture was stirred at rt for 3 h. The mixture was poured into ice-cold water (50 mL) and extracted with petroleum ether (2×50 mL). The organic layer was evaporated. The residue was stirred in n-pentane (20 mL) for 5 minutes. The solids were filtered, and the filtrate was evaporated under reduced pressure to give 2-fluoro-4-methoxy-1-vinylbenzene of as an off-white liquid (1.5 g, 76% yield). ¹H NMR (400 MHz, CDCl₃): δ 7.38 (t, 1H), 6.78 (dd, 1H), 6.66 (dd, 1H), 6.58 (dd, 1H), 5.68 (d, 1H), 5.23 (dd, 1H), 3.79 (s, 3H).

### (E)-2-Chloro-5-(2-fluoro-4-methoxystyryl)pyrimidine

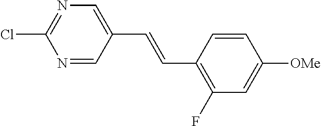 [SMILES: COc1ccc(/C=C/c2cnc(Cl)nc2)c(F)c1]

To a solution of 2-fluoro-4-methoxy-1-vinylbenzene (500 mg, 3.28 mmol) and 5-bromo-2-chloro pyrimidine (762 mg, 3.9 mmol) in acetonitrile (5 mL) was added DIPEA (1.1 mL, 6.6 mmol), and the mixture was degassed with argon for 5 min in a MW vial. To the reaction mixture was added Pd(OAc)₂ (72 mg, 0.32 mmol), and the resulting mixture was degassed with argon for 2 minutes. The reaction mixture was irradiated at 100° C. for 2 h, diluted with EtOAc (50 mL), and filtered through a CELITE® pad. The filtrate was washed with saturated aqueous brine solution (20 mL), dried over sodium sulfate, and evaporated to dryness under reduced pressure. The residue was purified by normal phase SiO₂ chromatography (0-10% EtOAc/petroleum ether) to give (E)-2-chloro-5-(2-fluoro-4-methoxystyryl)pyrimidine as an off-white liquid (200 mg, 23% yield, m/z: 265 [M+H]⁺ observed). ¹H NMR (400 MHz, CDCl₃): δ 8.71 (s, 2H), 7.48 (t, 1H), 7.27 (d, 1H), 6.91 (d, 1H), 6.73 (dd, 1H), 6.66 (dd, 1H), 3.83 (s, 3H).

### trans-2-Chloro-5-(2-(2-fluoro-4-methoxyphenyl)cyclopropyl)pyrimidine

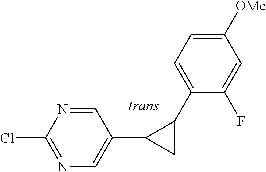 [SMILES: COc1ccc(C2CC2c2cnc(Cl)nc2)c(F)c1]

To a solution of (E)-2-chloro-5-(2-fluoro-4-methoxystyryl)pyrimidine (700 mg, 2.64 mmol) in CH₂Cl₂ (7 mL) at 0° C. was added Pd₃(OAc)₆ (180 mg, 0.26 mmol) and ethereal diazomethane [freshly prepared from N-methyl-N-nitroso urea (5.4 g, 52.8 mmol), KOH solution (50% in water, 60 mL) and Et₂O (60 mL) at 0° C.] and stirred at 0-5° C. for 20 h. The reaction mixture was filtered through a CELITE® pad, and the filtrate was concentrated under reduced pressure. The residue was dissolved in CH₂Cl₂ (7 mL) at 0° C., then Pd₃(OAc)₆ (180 mg, 0.26 mmol) was added followed by ethereal diazomethane [freshly prepared from N-methyl-N-nitroso urea (5.4 g, 52.8 mmol), KOH solution (50% in water, 60 mL) and Et₂O (60 mL) at 0° C.]. The mixture was stirred at 0-5° C. for 20 h. The reaction mixture was filtered through a CELITE® pad, and the filtrate was concentrated under reduced pressure. The residue was dissolved again in CH₂Cl₂ (7 mL) at 0° C., then Pd₃(OAc)₆ (180 mg, 0.26 mmol) was added, followed by ethereal diazomethane [freshly prepared from N-methyl-N-nitroso urea (5.4 g, 52.8 mmol), KOH solution (50% in water, 60 mL) and Et₂O (60 mL) at 0° C.]. The mixture was stirred at 0-5° C. for 20 h. The reaction mixture was filtered through a CELITE® pad and the filtrate was evaporated. The residue was purified by normal phase SiO₂ chromatography (0-10% EtOAc/petroleum ether) to give trans-2-chloro-5-(2-(2-fluoro-4-methoxyphenyl)cyclopropyl)pyrimidine as a pale yellow liquid (180 mg, m/z: 279 [M+H]⁺ observed), which was used in the next step without further purification.

### trans-5-(2-(2-Fluoro-4-methoxyphenyl)cyclopropyl)-2,2′-bipyrimidine

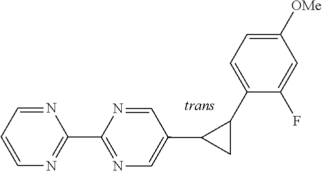 [SMILES: COc1ccc(C2CC2c2cnc(-c3ncccn3)nc2)c(F)c1]

To a solution of trans-2-chloro-5-(2-(2-fluoro-4-methoxyphenyl)cyclopropyl)pyrimidine (0.18 g, 0.64 mmol) in DMF (2 mL) was added 2-(tributylstannyl)pyrimidine (0.21 mL, 0.65 mmol), tetraethylammonium chloride (0.11 g, 0.66 mmol), and K₂CO₃ (0.18 g, 1.3 mmol) at rt, and the mixture was purged with N₂ gas for 10 min. Bis(triphenylphosphine)palladium(II) dichloride (32 mg, 0.046 mmol) was added and the mixture was purged with N₂ gas for 10 min. The reaction mixture was stirred at 100° C. for 24 h, cooled to rt, diluted with water (20 mL), and extracted with EtOAc (2×20 mL). The combined organic layer was washed with saturated aqueous brine solution (20 mL), dried over anhydrous sodium sulfate, filtered, and evaporated. The residue was purified by reverse phase HPLC to afford trans-5-(2-(2-fluoro-4-methoxyphenyl)cyclopropyl)-2,2′-bipyrimidine as a white solid (45 mg, 5% yield, m/z: 323 [M+H]+ observed). ¹H NMR (400 MHz, DMSO-d₆): δ 8.93 (d, 2H), 8.87 (s, 2H), 7.62 (t, 1H), 7.17 (t, 1H), 6.84-6.75 (m, 2H) 3.75 (s, 3H), 2.49-2.44 (m,1H), 2.33-2.28 (m, 1H), 1.72-1.60 (m, 2H).

### Example 2: trans-5-(2-(2-Fluoro-4-methoxyphenyl)cyclopropyl)-2,2′-bipyrimidine (single enantiomer I)

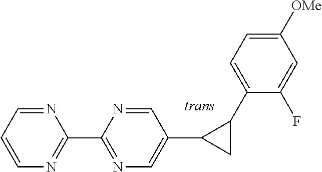 [SMILES: COc1ccc(C2CC2c2cnc(-c3ncccn3)nc2)c(F)c1]

### Example 3: trans-5-(2-(2-Fluoro-4-methoxyphenyl)cyclopropyl)-2,2′-bipyrimidine (single enantiomer II)

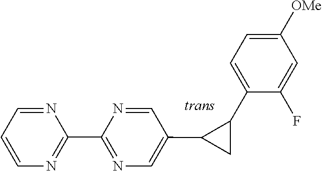 [SMILES: COc1ccc(C2CC2c2cnc(-c3ncccn3)nc2)c(F)c1]

A mixture of enantiomers (40 mg) was separated by SFC (supercritical fluid chromatography) on a CHIRALCEL® OD-H column using liquid CO₂ and iPrOH (1:1) to give trans-5-(2-(2-fluoro-4-methoxyphenyl)cyclopropyl)-2,2′-bipyrimidine (single enantiomer I) as a white solid (faster eluting enantiomer, 12 mg, 30%, m/z: 323 [M+H]⁺ observed), and trans-5-(2-(2-fluoro-4-methoxyphenyl)cyclopropyl)-2,2′-bipyrimidine (single enantiomer II) as a white solid (slower eluting enantiomer, 12 mg, 30%, m/z: 323 [M+H]⁺ observed).

### Example 2: trans-5-(2-(2-Fluoro-4-methoxyphenyl)cyclopropyl)-2,2′-bipyrimidine (single enantiomer I)

m/z: 323 [M+H]⁺ observed. ¹H NMR (400 MHz, DMSO-d₆): δ 8.93 (d, 2H), 8.87 (s, 2H), 7.62 (t, 1H), 7.17 (t, 1H), 6.84-6.75 (m, 2H) 3.75 (s, 3H), 2.49-2.44 (m,1H), 2.33-2.28 (m, 1H), 1.72-1.60 (m, 2H).

### Example 3: trans-5-(2-(2-Fluoro-4-methoxyphenyl)cyclopropyl)-2,2′-bipyrimidine (single enantiomer II)

m/z: 323 [M+H]⁺ observed. ¹H NMR (400 MHz, DMSO-d₆): δ 8.93 (d, 2H), 8.87 (s, 2H), 7.62 (t, 1H), 7.17 (t, 1H), 6.84-6.75 (m, 2H) 3.75 (s, 3H), 2.49-2.44 (m,1H), 2.33-2.28 (m, 1H), 1.72-1.60 (m, 2H).

The following examples were prepared in a similar manner as trans-5-(2-(2-fluoro-4-methoxyphenyl)cyclopropyl)-2,2′-bipyrimidine from an appropriately substituted benzaldehyde and an appropriately substituted 2-chloropyrimidine.

### Example 4: trans-4-(2-(3,4-Difluorophenyl)cyclopropyl)-2,2′-bipyrimidine

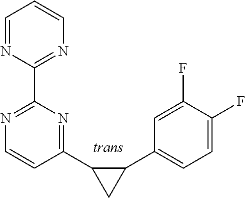 [SMILES: Fc1ccc(C2CC2c2ccnc(-c3ncccn3)n2)cc1F]

m/z: 311 [M+H]⁺ observed. ¹H NMR (400 MHz, DMSO-d₆): δ 8.99 (d, 2H), 8.79 (s, 1H), 7.65-7.61 (m, 2H), 7.37-7.30 (m, 2H), 7.13-7.11 (m, 1H), 2.64-2.59 (m, 2H), 1.85-1.82 (m, 1H), 1.68-1.65 (m, 1H).

### Example 5: trans-4-(2-(3,4-Difluorophenyl)cyclopropyl)-2,2′-bipyrimidine (single enantiomer I)

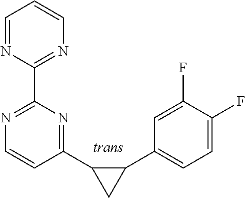 [SMILES: Fc1ccc(C2CC2c2ccnc(-c3ncccn3)n2)cc1F]

### Example 6: trans-4-(2-(3,4-Difluorophenyl)cyclopropyl)-2,2′-bipyrimidine (single enantiomer II)

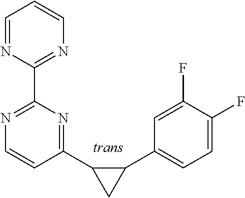 [SMILES: Fc1ccc(C2CC2c2ccnc(-c3ncccn3)n2)cc1F]

A mixture of enantiomers (140 mg) was separated by SFC (supercritical fluid chromatography) on a CHIRALCEL® OD-H column using liquid CO₂ and EtOH (55:45) to give trans-4-(2-(3,4-difluorophenyl)cyclopropyl)-2,2′-bipyrimidine (single enantiomer I) as a white solid (faster eluting enantiomer, 39 mg, 28%, m/z: 311 [M+H]⁺ observed), and trans-4-(2-(3,4-difluorophenyl)cyclopropyl)-2,2′-bipyrimidine (single enantiomer II) as a pale yellow solid (slower eluting enantiomer, 37 mg, 26%, m/z: 311 [M+H]⁺ observed).

### Example 5: trans-4-(2-(3,4-Difluorophenyl)cyclopropyl)-2,2′-bipyrimidine (single enantiomer I)

m/z: 311 [M+H]⁺ observed. ¹H NMR (400 MHz, DMSO-d₆): δ 8.99 (d, 2H), 8.79 (s, 1H), 7.65-7.61 (m, 2H), 7.37-7.30 (m, 2H), 7.13-7.11 (m, 1H), 2.64-2.59 (m, 2H), 1.85-1.82 (m, 1H), 1.68-1.65 (m, 1H).

### Example 6: trans-4-(2-(3,4-Difluorophenyl)cyclopropyl)-2,2′-bipyrimidine (single enantiomer II)

m/z: 311 [M+H]⁺ observed. ¹H NMR (400 MHz, DMSO-d₆): δ 8.99 (d, 2H), 8.79 (s, 1H), 7.65-7.61 (m, 2H), 7.37-7.30 (m, 2H), 7.13-7.11 (m, 1H), 2.64-2.59 (m, 2H), 1.85-1.82 (m, 1H), 1.68-1.65 (m, 1H).

### Example 7: trans-5-(2-(4-Fluoro-2-methoxyphenyl)cyclopropyl)-2,2′-bipyrimidine

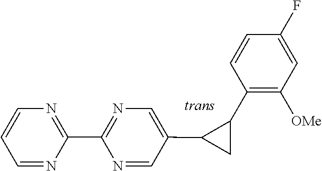 [SMILES: COc1cc(F)ccc1C1CC1c1cnc(-c2ncccn2)nc1]

m/z: 323 [M+H]⁺ observed. ¹H NMR (400 MHz, DMSO-d₆): δ 8.95 (d, 2H), 8.83 (s, 2H), 7.60-7.58 (m, 1H), 7.10-7.06 (m, 1H), 6.88-6.84 (m, 1H), 6.72-6.67 (m, 1H), 3.77 (s, 3H), 2.47-2.38 (m, 1H), 2.19-2.14 (m, 1H), 1.65-1.54 (m, 2H).

### Example 8: trans-5-(2-(4-Fluoro-2-methoxyphenyl)cyclopropyl)-2,2′-bipyrimidine (single enantiomer I)

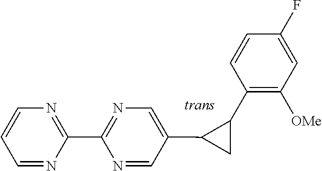 [SMILES: COc1cc(F)ccc1C1CC1c1cnc(-c2ncccn2)nc1]

### Example 9: trans-5-(2-(4-Fluoro-2-methoxyphenyl)cyclopropyl)-2,2′-bipyrimidine (single enantiomer II)

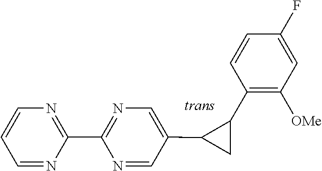 [SMILES: COc1cc(F)ccc1C1CC1c1cnc(-c2ncccn2)nc1]

A mixture of enantiomers (120 mg) was separated by chiral HPLC on a LUX® Amylose-2 column using n-hexane and EtOH (70:30) to give trans-5-(2-(4-fluoro-2-methoxyphenyl)cyclopropyl)-2,2′-bipyrimidine (single enantiomer I) as a white solid (faster eluting enantiomer, 21 mg, 28%, m/z: 323 [M+H]⁺ observed), and trans-5-(2-(4-fluoro-2-methoxyphenyl)cyclopropyl)-2,2′-bipyrimidine (single enantiomer II) as a pale yellow solid (slower eluting enantiomer, 20 mg, 26%, m/z: 323 [M+H]⁺ observed).

### Example 8: trans-5-(2-(4-Fluoro-2-methoxyphenyl)cyclopropyl)-2,2′-bipyrimidine (single enantiomer I)

m/z: 323 [M+H]⁺ observed. ¹H NMR (400 MHz, DMSO-d₆): δ 8.95 (d, 2H), 8.83 (s, 2H), 7.60-7.58 (m, 1H), 7.10-7.06 (m, 1H), 6.88-6.84 (m, 1H), 6.72-6.67 (m, 1H), 3.77 (s, 3H), 2.47-2.38 (m, 1H), 2.19-2.14 (m, 1H), 1.65-1.54 (m, 2H).

### Example 9: trans-5-(2-(4-Fluoro-2-methoxyphenyl)cyclopropyl)-2,2′-bipyrimidine (single enantiomer II)

m/z: 323 [M+H]⁺ observed. ¹H NMR (400 MHz, DMSO-d₆): δ 8.95 (d, 2H), 8.83 (s, 2H), 7.60-7.58 (m, 1H), 7.10-7.06 (m, 1H), 6.88-6.84 (m, 1H), 6.72-6.67 (m, 1H), 3.77 (s, 3H), 2.47-2.38 (m, 1H), 2.19-2.14 (m, 1H), 1.65-1.54 (m, 2H).

### Example 10: trans 5-(2-(4-Fluoro-3-methoxyphenyl)cyclopropyl)-2,2′-bipyrimidine (single enantiomer I)

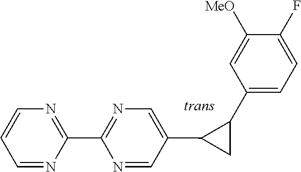 [SMILES: COc1cc(C2CC2c2cnc(-c3ncccn3)nc2)ccc1F]

### Example 11: trans-5-(2-(4-Fluoro-3-methoxyphenyl)cyclopropyl)-2,2′-bipyrimidine (single enantiomer II)

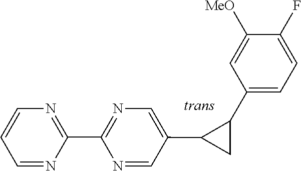 [SMILES: COc1cc(C2CC2c2cnc(-c3ncccn3)nc2)ccc1F]

A mixture of enantiomers (130 mg) was separated by SFC (supercritical fluid chromatography) on a CHIRALPAK® IG column using liquid CO₂ and MeOH [50:50; 0.1% methanolic NH₃ as modifier)] to give trans-5-(2-(4-fluoro-3-methoxyphenyl)cyclopropyl)-2,2′-bipyrimidine (single enantiomer I) as a white solid (faster eluting enantiomer, 32 mg, 25%, m/z: 323 [M+H]⁺ observed), and trans-5-(2-(4-fluoro-2-methoxyphenyl)cyclopropyl)-2,2′-bipyrimidine (single enantiomer II) as a white solid (slower eluting enantiomer, 25 mg, 19%, m/z: 323 [M+H]⁺ observed).

### Example 10: trans-5-(2-(4-Fluoro-3-methoxyphenyl)cyclopropyl)-2,2′-bipyrimidine (single enantiomer I)

m/z: 323 [M+H]⁺ observed. ¹H NMR (400 MHz, DMSO-d₆): δ 8.98 (d, 2H), 8.84 (s, 2H), 7.63-7.61 (t, 1H), 7.15-7.10 (m, 1H), 7.03-7.01 (m, 1H) ,6.81-6.77 (m, 1H), 3.85 (s, 3H), 2.53-2.49 (m, 1H), 2.37-2.35 (m, 1H), 1.73-1.64 (m, 2H).

### Example 11: trans-5-(2-(4-Fluoro-3-methoxyphenyl)cyclopropyl)-2,2′-bipyrimidine (single enantiomer II)

m/z: 323 [M+H]⁺ observed. ¹H NMR (400 MHz, DMSO-d₆): δ 8.98 (d, 2H), 8.84 (s, 2H), 7.63-7.61 (t, 1H), 7.15-7.10 (m, 1H), 7.03-7.01 (m, 1H) ,6.81-6.77 (m, 1H), 3.85 (s, 3H), 2.53-2.49 (m, 1H), 2.37-2.35 (m, 1H), 1.73-1.64 (m, 2H).

### Example 12: trans-5-(2-(3-Fluoro-4-methoxyphenyl)cyclopropyl)-2,2′-bipyrimidine (single enantiomer I)

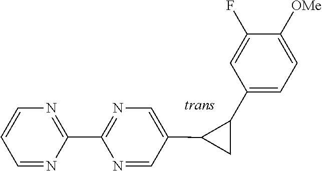 [SMILES: COc1ccc(C2CC2c2cnc(-c3ncccn3)nc2)cc1F]

### Example 13: trans-5-(2-(3-Fluoro-4-methoxyphenyl)cyclopropyl)-2,2′-bipyrimidine (single enantiomer II)

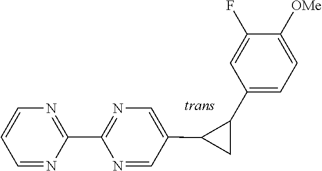 [SMILES: COc1ccc(C2CC2c2cnc(-c3ncccn3)nc2)cc1F]

A mixture of enantiomers (81 mg) was separated by SFC (supercritical fluid chromatography) on a CHIRALPAK® OD-H column using liquid CO₂ and MeOH (55:45) to give trans-5-(2-(3-fluoro-4-methoxyphenyl)cyclopropyl)-2,2′-bipyrimidine (single enantiomer I) as an off-white solid (faster eluting enantiomer, 23 mg, 28%, m/z: 323 [M+H]⁺ observed), and trans-5-(2-(3-fluoro-4-methoxyphenyl)cyclopropyl)-2,2′-bipyrimidine (single enantiomer II) as an off-white solid (slower eluting enantiomer, 18 mg, 22%, m/z: 323 [M+H]⁺ observed).

### Example 12: trans-5-(2-(3-Fluoro-4-methoxyphenyl)cyclopropyl)-2,2′-bipyrimidine (single enantiomer I)

m/z: 323 [M+H]⁺ observed. ¹H NMR (400 MHz, DMSO-d₆): δ 8.98 (d, 2H), 8.83 (s, 2H), 7.62 (t, 1H), 7.11-7.02 (m, 3H), 3.81 (s, 3H), 2.49-2.45 (m, 1H), 2.33-2.28 (m, 1H),1.72-1.59 (m, 2H).

### Example 13: trans-5-(2-(3-Fluoro-4-methoxyphenyl)cyclopropyl)-2,2′-bipyrimidine (single enantiomer II)

m/z: 323 [M+H]⁺ observed. ¹H NMR (400 MHz, DMSO-d₆): δ 8.98 (d, 2H), 8.83 (s, 2H), 7.62 (t, 1H), 7.11-7.02 (m, 3H), 3.81 (s, 3H), 2.49-2.45 (m, 1H), 2.33-2.28 (m, 1H),1.72-1.59 (m, 2H).

### Example 14: trans-5-(2-(3,4-Difluorophenyl)cyclopropyl)-2,2′-bipyrimidine (single enantiomer I)

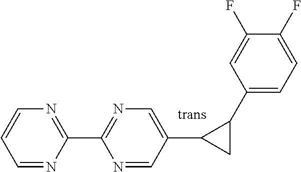 [SMILES: Fc1ccc(C2CC2c2cnc(-c3ncccn3)nc2)cc1F]

### Example 15: trans-5-(2-(3,4-Difluorophenyl)cyclopropyl)-2,2′-bipyrimidine (single enantiomer II)

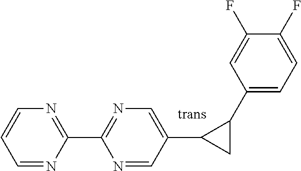 [SMILES: Fc1ccc(C2CC2c2cnc(-c3ncccn3)nc2)cc1F]

A mixture of enantiomers (130 mg) was separated by chiral HPLC on a CHIRALPAK® AD-H column using n-hexane and EtOH (25:75) to give trans-5-(2-(3,4-difluorophenyl)cyclopropyl)-2,2′-bipyrimidine (single enantiomer I) as an off-white solid (faster eluting enantiomer, 25 mg, 19%, m/z: 311 [M+H]⁺ observed), and trans-5-(2-(3,4-difluorophenyl)cyclopropyl)-2,2′-bipyrimidine (single enantiomer II) as an off-white solid (slower eluting enantiomer, 45 mg, 35%, m/z: 311 [M+H]+ observed).

### Example 14: trans-5-(2-(3,4-Difluorophenyl)cyclopropyl)-2,2′-bipyrimidine (single enantiomer I)

m/z: 311 [M+H]⁺ observed. ¹H NMR (400 MHz, DMSO-d₆): δ 8.99 (d, 2H), 8.84 (s, 2H), 7.63 (t, 1H), 7.40-7.30 (m, 2H), 7.14-7.11 (m, 1H), 2.54 (s, 1H), 2.40-2.36 (m, 1H), 1.78-1.73 (m, 1H), 1.68-1.63 (m, 1H).

### Example 15: trans-5-(2-(3,4-Difluorophenyl)cyclopropyl)-2,2′-bipyrimidine (single enantiomer II)

m/z: 311 [M+H]⁺ observed. ¹H NMR (400 MHz, DMSO-d₆): δ 8.99 (d, 2H), 8.84 (s, 2H), 7.63 (t, 1H), 7.40-7.30 (m, 2H), 7.14-7.11 (m, 1H), 2.54 (s, 1H), 2.40-2.36 (m, 1H), 1.78-1.73 (m, 1H), 1.68-1.63 (m, 1H).

### Example 16: trans-5-(2-(4-Fluoro-3-methoxyphenyl)cyclopropyl)-4-methyl-2,2′-bipyrimidine

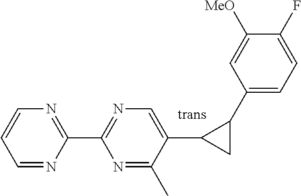 [SMILES: COc1cc(C2CC2c2cnc(-c3ncccn3)nc2C)ccc1F]

m/z: 337 [M+H]⁺ observed. ¹H NMR (400 MHz, DMSO-d₆): δ 8.98 (d, 2H), 8.67 (s, 1H), 7.63-7.60 (m, 1H), 7.15-7.06 (m, 2H), 6.86-6.83 (m, 1H), 3.86 (s, 3H), 2.59 (m, 3H), 2.36-2.30 (m, 2H), 1.67-1.52 (m, 2H).

A mixture of enantiomers (42 mg) was separated by SFC (supercritical fluid chromatography) on a CHIRALPAK® OD-H column using liquid CO₂ and MeOH (60:40) to give trans-5-(2-(4-fluoro-3-methoxyphenyl)cyclopropyl)-4-methyl-2,2′-bipyrimidine (single enantiomer I) as an off-white solid (faster eluting enantiomer, 10 mg, 24%, m/z: 337 [M+H]⁺ observed), and trans-5-(2-(4-fluoro-3-methoxyphenyl)cyclopropyl)-4-methyl-2,2′-bipyrimidine (single enantiomer II) as an off-white solid (slower eluting enantiomer, 8 mg, 19%, m/z: 337 [M+H]⁺ observed).

### Example 17: trans-5-(2-(4-Fluoro-3-methoxyphenyl)cyclopropyl)-4-methyl-2,2′-bipyrimidine (single enantiomer I)

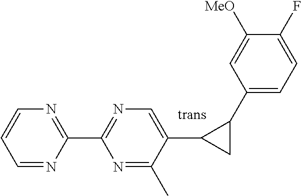 [SMILES: COc1cc(C2CC2c2cnc(-c3ncccn3)nc2C)ccc1F]

m/z: 337 [M+H]⁺ observed. ¹H NMR (400 MHz, DMSO-d₆): δ 8.98 (d, 2H), 8.67 (s, 1H), 7.63-7.60 (m, 1H), 7.15-7.06 (m, 2H), 6.86-6.83 (m, 1H), 3.86 (s, 3H), 2.59 (m, 3H), 2.36-2.30 (m, 2H), 1.67-1.52 (m, 2H).

### Example 18: trans-5-(2-(4-Fluoro-3-methoxyphenyl)cyclopropyl)-4-methyl-2,2′-bipyrimidine (single enantiomer II)

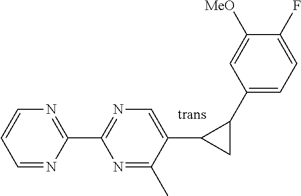 [SMILES: COc1cc(C2CC2c2cnc(-c3ncccn3)nc2C)ccc1F]

m/z: 337 [M+H]⁺ observed. ¹H NMR (400 MHz, DMSO-d₆): δ 8.98 (d, 2H), 8.67 (s, 1H), 7.63-7.60 (m, 1H), 7.15-7.06 (m, 2H), 6.86-6.83 (m, 1H), 3.86 (s, 3H), 2.59 (m, 3H), 2.36-2.30 (m, 2H), 1.67-1.52 (m, 2H).

### Example 19: trans-5-(2-(4-Fluoro-3-(pyrrolidin-1-yl)phenyl)cyclopropyl)-2,2′-bipyrimidine (single enantiomer I)

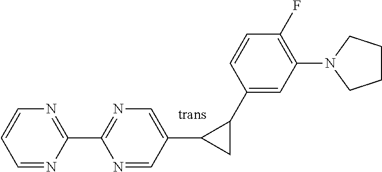 [SMILES: Fc1ccc(C2CC2c2cnc(-c3ncccn3)nc2)cc1N1CCCC1]

### Example 20: trans-5-(2-(4-Fluoro-3-(pyrrolidin-1-yl)phenyl)cyclopropyl)-2,2′-bipyrimidine (single enantiomer II)

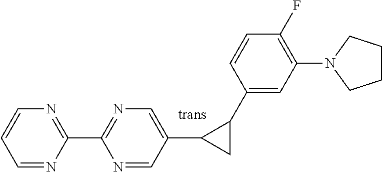 [SMILES: Fc1ccc(C2CC2c2cnc(-c3ncccn3)nc2)cc1N1CCCC1]

A mixture of enantiomers (100 mg) was separated by SFC (supercritical fluid chromatography) on a CHIRALPAK® OD-H column using liquid CO₂ and EtOH (50:50) to give trans-5-(2-(4-fluoro-3-methoxyphenyl)cyclopropyl)-4-methyl-2,2′-bipyrimidine (single enantiomer I) as an off-white solid (faster eluting enantiomer, 26 mg, 26%, m/z: 362 [M+H]⁺ observed), and trans-5-(2-(4-fluoro-3-methoxyphenyl)cyclopropyl)-4-methyl-2,2′-bipyrimidine (single enantiomer II) as an off-white solid (slower eluting enantiomer, 25 mg, 25%, m/z: 362 [M+H]⁺ observed).

### Example 19: trans-5-(2-(4-Fluoro-3-(pyrrolidin-1-yl)phenyl)cyclopropyl)-2,2′-bipyrimidine (single enantiomer I)

m/z: 362 [M+H]⁺ observed. ¹H NMR (400 MHz, DMSO-d₆): δ 8.99 (d, 2H), 8.83 (s, 2H), 7.63-7.60 (m, 1H), 6.99-6.94 (m, 1H), 6.58-6.56 (m, 1H), 6.50-6.48 (m, 1H), 3.31 (br m, 4H), 2.46-2.42 (m, 1H), 2.33-2.30 (m, 1H), 1.90-1.87 (m, 4H), 1.68-1.66 (m, 1H), 1.61-1.57 (m, 1H).

### Example 20: trans-5-(2-(4-Fluoro-3-(pyrrolidin-1-yl)phenyl)cyclopropyl)-2,2′-bipyrimidine (single enantiomer II)

m/z: 362 [M+H]⁺ observed. ¹H NMR (400 MHz, DMSO-d₆): δ 8.99 (d, 2H), 8.83 (s, 2H), 7.63-7.60 (m, 1H), 6.99-6.94 (m, 1H), 6.58-6.56 (m, 1H), 6.50-6.48 (m, 1H), 3.31 (br m, 4H), 2.46-2.42 (m, 1H), 2.33-2.30 (m, 1H), 1.90-1.87 (m, 4H), 1.68-1.66 (m, 1H), 1.61-1.57 (m, 1H).

### Example 21: trans-5-(2-(4-Fluoro-3-(2-methoxyethoxy)phenyl)cyclopropyl)-2,2′-bipyrimidine (single enantiomer I)

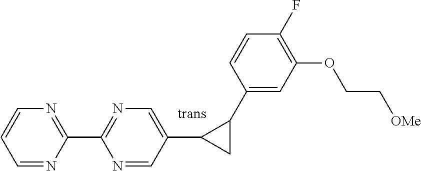 [SMILES: COCCOc1cc(C2CC2c2cnc(-c3ncccn3)nc2)ccc1F]

### Example 22: trans-5-(2-(4-Fluoro-3-(2-methoxyethoxy)phenyl)cyclopropyl)-2,2′-bipyrimidine (single enantiomer II)

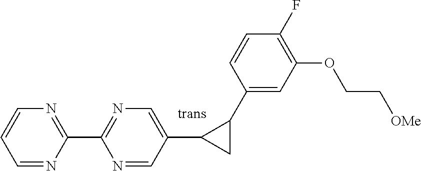 [SMILES: COCCOc1cc(C2CC2c2cnc(-c3ncccn3)nc2)ccc1F]

A mixture of enantiomers (230 mg) was separated by SFC (supercritical fluid chromatography) on a CHIRALPAK® OD-H column using liquid CO₂ and EtOH (55:45) to give trans-5-(2-(4-fluoro-3-(2-methoxyethoxy)phenyl)cyclopropyl)-2,2′-bipyrimidine (single enantiomer I) as an pale brown solid (faster eluting enantiomer, 68 mg, 29%, m/z: 367 [M+H]⁺ observed), and trans-5-(2-(4-fluoro-3-(2-methoxyethoxy)phenyl)cyclopropyl)-2,2′-bipyrimidine (single enantiomer II) as an pale brown solid (slower eluting enantiomer, 66 mg, 28%, m/z: 367 [M+H]⁺ observed).

### Example 21: trans-5-(2-(4-Fluoro-3-(2-methoxyethoxy)phenyl)cyclopropyl)-2,2′-bipyrimidine (single enantiomer I)

m/z: 367 [M+H]⁺ observed. ¹H NMR (400 MHz, DMSO-d₆): δ 8.97 (d, 2H), 8.84 (s, 2H), 7.63-7.61 (m, 1H), 7.15-7.10 (m, 1H), 7.03-7.01 (m, 1H), 6.82-6.79 (m, 1H), 4.19 (m, 2H), 3.67 (m, 2H), 3.32 (s, 3H), 2.36-2.34 (m, 2H), 1.72-1.64 (m, 2H).

### Example 22: trans-5-(2-(4-Fluoro-3-(2-methoxyethoxy)phenyl)cyclopropyl)-2,2′-bipyrimidine (single enantiomer II)

m/z: 367 [M+H]⁺ observed. ¹H NMR (400 MHz, DMSO-d₆): δ 8.97 (d, 2H), 8.84 (s, 2H), 7.63-7.61 (m, 1H), 7.15-7.10 (m, 1H), 7.03-7.01 (m, 1H), 6.82-6.79 (m, 1H), 4.19 (m, 2H), 3.67 (m, 2H), 3.32 (s, 3H), 2.36-2.34 (m, 2H), 1.72-1.64 (m, 2H).

### Example 23: trans-5-(2-(3-Fluoro-4-(2-methoxyethoxy)phenyl)cyclopropyl)-2,2′-bipyrimidine (single enantiomer I)

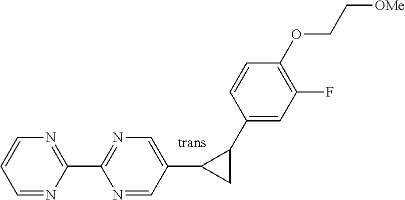 [SMILES: COCCOc1ccc(C2CC2c2cnc(-c3ncccn3)nc2)cc1F]

### Example 24: trans-5-(2-(3-Fluoro-4-(2-methoxyethoxy)phenyl)cyclopropyl)-2,2′-bipyrimidine (single enantiomer II)

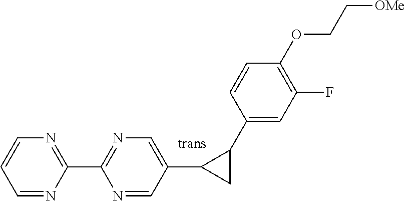 [SMILES: COCCOc1ccc(C2CC2c2cnc(-c3ncccn3)nc2)cc1F]

A mixture of enantiomers (120 mg) was separated by SFC (supercritical fluid chromatography) on a CHIRALPAK® OD-H column using liquid CO₂ and MeOH (50:50) to give trans-5-(2-(3-fluoro-4-(2-methoxyethoxy)phenyl)cyclopropyl)-2,2′-bipyrimidine (single enantiomer I) as an white solid (faster eluting enantiomer, 25 mg, 21%, m/z: 367 [M+H]⁺ observed), and trans-5-(2-(3-fluoro-4-(2-methoxyethoxy)phenyl)cyclopropyl)-2,2′-bipyrimidine (single enantiomer II) as an white solid (slower eluting enantiomer, 23 mg, 19%, m/z: 367 [M+H]⁺ observed).

### Example 23: trans-5-(2-(3-Fluoro-4-(2-methoxyethoxy)phenyl)cyclopropyl)-2,2′-bipyrimidine (single enantiomer I)

m/z: 367 [M+H]⁺ observed. ¹H NMR (400 MHz, DMSO-d₆): δ 8.99 (d, 2H), 8.83 (s, 2H), 7.63-7.61 (m, 1H), 7.12-7.08 (m, 2H), 7.03-7.00 (m, 1H), 4.15-4.13 (m, 2H), 3.67-3.64 (m, 2H), 3.31 (s, 3H), 2.50-2.45 (m, 1H), 2.33-2.29 (m, 1H), 1.76-1.67 (m, 1H), 1.62-1.57 (m, 1H).

### Example 24: trans-5-(2-(3-fluoro-4-(2-methoxyethoxy)phenyl)cyclopropyl)-2,2′-bipyrimidine (single enantiomer II)

m/z: 367 [M+H]⁺ observed. ¹H NMR (400 MHz, DMSO-d₆): δ 8.99 (d, 2H), 8.83 (s, 2H), 7.63-7.61 (m, 1H), 7.12-7.08 (m, 2H), 7.03-7.00 (m, 1H), 4.15-4.13 (m, 2H), 3.67-3.64 (m, 2H), 3.31 (s, 3H), 2.50-2.45 (m, 1H), 2.33-2.29 (m, 1H), 1.76-1.67 (m, 1H), 1.62-1.57 (m, 1H).

### Example 25: trans-5-(2-(3-(Cyclopropylmethoxy)-4-fluorophenyl)cyclopropyl)-2,2′-bipyrimidine (single enantiomer I)

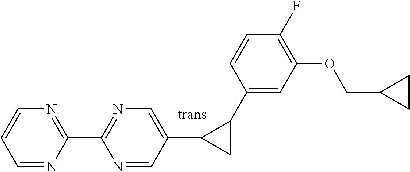 [SMILES: Fc1ccc(C2CC2c2cnc(-c3ncccn3)nc2)cc1OCC1CC1]

### Example 26: trans-5-(2-(3-(Cyclopropylmethoxy)-4-fluorophenyl)cyclopropyl)-2,2′-bipyrimidine (single enantiomer II)

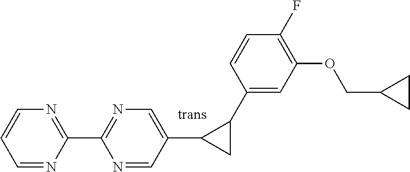 [SMILES: Fc1ccc(C2CC2c2cnc(-c3ncccn3)nc2)cc1OCC1CC1]

A mixture of enantiomers (120 mg) was separated by SFC (supercritical fluid chromatography) on a CHIRALPAK® OD-H column using liquid CO₂ and MeOH (60:40) to give trans-5-(2-(3-(cyclopropylmethoxy)-4-fluorophenyl)cyclopropyl)-2,2′-bipyrimidine (single enantiomer I) as a red solid (faster eluting enantiomer, 21 mg, 17%, m/z: 363 [M+H]⁺ observed), and trans-5-(2-(3-(cyclopropylmethoxy)-4-fluorophenyl)cyclopropyl)-2,2′-bipyrimidine (single enantiomer II) as an white solid (slower eluting enantiomer, 23 mg, 19%, m/z: 363 [M+H]⁺ observed).

### Example 25: trans-5-(2-(3-(Cyclopropylmethoxy)-4-fluorophenyl)cyclopropyl)-2,2′-bipyrimidine (single enantiomer I)

m/z: 363 [M+H]⁺ observed. ¹H NMR (400 MHz, DMSO-d₆): δ 8.98 (d, 2H), 8.83 (s, 2H), 7.63-7.61 (m, 1H), 7.14-7.09 (m, 1H), 6.99-6.96 (m, 1H), 6.78-6.77 (m, 1H), 3.91 (d, 2H), 2.52-2.48 (m, 1H), 2.35-2.32 (m, 1H), 1.71-1.62 (m, 2H), 1.24-1.23 (m, 1H), 0.60-0.56 (m, 2H), 0.34-0.30 (m, 2H).

### Example 26: trans-5-(2-(3-(Cyclopropylmethoxy)-4-fluorophenyl)cyclopropyl)-2,2′-bipyrimidine (single enantiomer II)

m/z: 363 [M+H]⁺ observed. ¹H NMR (400 MHz, DMSO-d₆): δ 8.98 (d, 2H), 8.83 (s, 2H), 7.63-7.61 (m, 1H), 7.14-7.09 (m, 1H), 6.99-6.96 (m, 1H), 6.78-6.77 (m, 1H), 3.91 (d, 2H), 2.52-2.48 (m, 1H), 2.35-2.32 (m, 1H), 1.71-1.62 (m, 2H), 1.24-1.23 (m, 1H), 0.60-0.56 (m, 2H), 0.34-0.30 (m, 2H).

### Example 27: trans-5-(2-(3-(2,2-Difluoroethoxy)-4-fluorophenyl)cyclopropyl)-2,2′-bipyrimidine (single enantiomer I)

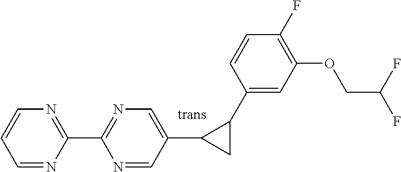 [SMILES: Fc1ccc(C2CC2c2cnc(-c3ncccn3)nc2)cc1OCC(F)F]

### Example 28: trans-5-(2-(3-(2,2-Difluoroethoxy)-4-fluorophenyl)cyclopropyl)-2,2′-bipyrimidine (single enantiomer II)

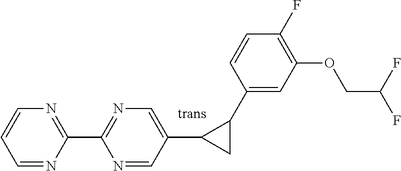 [SMILES: Fc1ccc(C2CC2c2cnc(-c3ncccn3)nc2)cc1OCC(F)F]

A mixture of enantiomers (100 mg) was separated by SFC (supercritical fluid chromatography) on a CHIRALPAK® OD-H column using liquid CO₂ and MeOH (60:40) to give trans-5-(2-(3-(2,2-difluoroethoxy)-4-fluorophenyl)cyclopropyl)-2,2′-bipyrimidine (single enantiomer I) as a brown solid (faster eluting enantiomer, 35 mg, 35%, m/z: 373 [M+H]⁺ observed), and trans-5-(2-(3-(2,2-difluoroethoxy)-4-fluorophenyl)cyclopropyl)-2,2′-bipyrimidine (single enantiomer II) as a brown solid (slower eluting enantiomer, 40 mg, 40%, m/z: 373 [M+H]⁺ observed).

### Example 27: trans-5-(2-(3-(2,2-Difluoroethoxy)-4-fluorophenyl)cyclopropyl)-2,2′-bipyrimidine (single enantiomer I)

m/z: 373 [M+H]⁺ observed. ¹H NMR (400 MHz, DMSO-d₆): δ 8.98 (d, 2H), 8.84 (s, 2H), 7.63-7.61 (m, 1H), 7.20-7.06 (m, 2H), 6.90-6.87 (m, 1H), 6.56-6.27 (m, 1H), 4.46-4.38 (m, 2H), 2.53-2.50 (m, 1H), 2.39-2.32 (m, 1H), 1.81-1.65 (m, 2H).

### Example 28: trans-5-(2-(3-(2,2-Difluoroethoxy)-4-fluorophenyl)cyclopropyl)-2,2′-bipyrimidine (single enantiomer II)

m/z: 373 [M+H]⁺ observed. ¹H NMR (400 MHz, DMSO-d₆): δ 8.98 (d, 2H), 8.84 (s, 2H), 7.63-7.61 (m, 1H), 7.20-7.06 (m, 2H), 6.90-6.87 (m, 1H), 6.56-6.27 (m, 1H), 4.46-4.38 (m, 2H), 2.53-2.50 (m, 1H), 2.39-2.32 (m, 1H), 1.81-1.65 (m, 2H).

### Example 29: trans-5-(2-(3-Chloro-4-fluorophenyl)cyclopropyl)-2,2′-bipyrimidine (single enantiomer I)

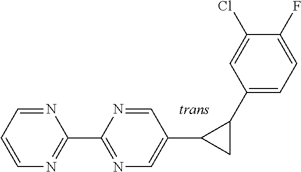 [SMILES: Fc1ccc(C2CC2c2cnc(-c3ncccn3)nc2)cc1Cl]

### Example 30: trans-5-(2-(3-Chloro-4-fluorophenyl)cyclopropyl)-2,2′-bipyrimidine (single enantiomer II)

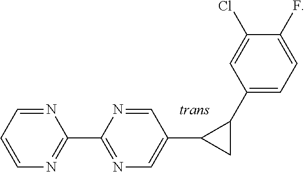 [SMILES: Fc1ccc(C2CC2c2cnc(-c3ncccn3)nc2)cc1Cl]

A mixture of enantiomers (180 mg) was separated by SFC (supercritical fluid chromatography) on a CHIRALPAK® IC column using liquid CO₂ and EtOH [50:50; 0.1% methanolic NH₃ as modifier)] to give trans-5-(2-(3-chloro-4-fluorophenyl)cyclopropyl)-2,2′-bipyrimidine (single enantiomer I) as an off-white solid (faster eluting enantiomer, 55 mg, 31%, m/z: 327 [M+H]⁺ observed), and trans-5-(2-(3-chloro-4-fluorophenyl)cyclopropyl)-2,2′-bipyrimidine (single enantiomer II) as an off-white solid (slower eluting enantiomer, 55 mg, 31%, m/z: 327 [M+H]⁺ observed).

### Example 29: trans-5-(2-(3-Chloro-4-fluorophenyl)cyclopropyl)-2,2′-bipyrimidine (single enantiomer I)

m/z: 327 [M+H]⁺ observed. ¹H NMR (400 MHz, DMSO-d₆): δ 8.99 (d, 2H), 8.84 (s, 2H), 7.63 (t, 1H), 7.49-7.46 (m, 1H), 7.38-7.28 (m, 1H), 7.29-7.25 (m, 1H), 2.59-2.54 (m, 1H), 2.43-2.37 (m 1H), 1.77-1.64 (m, 2H).

### Example 30: trans-5-(2-(3-Chloro-4-fluorophenyl)cyclopropyl)-2,2′-bipyrimidine (single enantiomer II)

m/z: 327 [M+H]⁺ observed. ¹H NMR (400 MHz, DMSO-d₆): δ 8.99 (d, 2H), 8.84 (s, 2H), 7.63 (t, 1H), 7.49-7.46 (m, 1H), 7.38-7.28 (m, 1H), 7.29-7.25 (m, 1H), 2.59-2.54 (m, 1H), 2.43-2.37 (m 1H), 1.77-1.64 (m, 2H).

### Example 31: trans-5-(2-(3,4-Dimethoxyphenyl)cyclopropyl)-2,2′-bipyrimidine (single enantiomer I)

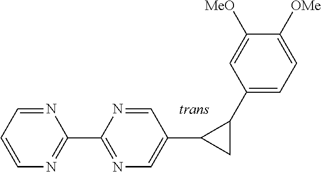 [SMILES: COc1ccc(C2CC2c2cnc(-c3ncccn3)nc2)cc1OC]

### Example 32: trans-5-(2-(3,4-Dimethoxyphenyl)cyclopropyl)-2,2′-bipyrimidine (single enantiomer II)

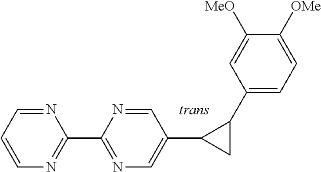 [SMILES: COc1ccc(C2CC2c2cnc(-c3ncccn3)nc2)cc1OC]

A mixture of enantiomers (200 mg) was separated by SFC (supercritical fluid chromatography) on a CHIRALPAK® OJ-H column using liquid CO₂ and MeOH [85:15; 0.1% methanolic NH₃ as modifier)] to give trans-5-(2-(3,4-dimethoxyphenyl)cyclopropyl)-2,2′-bipyrimidine (single enantiomer I) as an off-white solid (faster eluting enantiomer, 60 mg, 30%, m/z: 335 [M+H]⁺ observed), and trans-5-(2-(3,4-dimethoxyphenyl)cyclopropyl)-2,2′-bipyrimidine (single enantiomer II) as an off-white solid (slower eluting enantiomer, 63 mg, 32%, m/z: 335 [M+H]⁺ observed).

### Example 31: trans-5-(2-(3,4-Dimethoxyphenyl)cyclopropyl)-2,2′-bipyrimidine (single enantiomer I)

m/z: 335 [M+H]⁺ observed. ¹H NMR (400 MHz, DMSO-d₆): δ 8.99 (d, 2H), 8.83 (s, 2H), 7.62 (t, 1H), 6.88 (d, 1H), 6.82 (s, 1H), 6.76-6.74 (m, 1H), 3.76 (s, 3H), 3.72 (s, 3H), 2.50-2.43 (m, 1H), 2.32-2.27 (m, 1H), 1.70-1.59 (m, 2H).

### Example 32: trans-5-(2-(3,4-Dimethoxyphenyl)cyclopropyl)-2,2′-bipyrimidine (single enantiomer II)

m/z: 335 [M+H]⁺ observed. ¹H NMR (400 MHz, DMSO-d₆): δ 8.99 (d, 2H), 8.83 (s, 2H), 7.62 (t, 1H), 6.88 (d, 1H), 6.82 (s, 1H), 6.76-6.74 (m, 1H), 3.76 (s, 3H), 3.72 (s, 3H), 2.50-2.43 (m, 1H), 2.32-2.27 (m, 1H), 1.70-1.59 (m, 2H).

### Example 33: trans-5-(2-(4-Chloro-3-methoxyphenyl)cyclopropyl)-2,2′-bipyrimidine (single enantiomer I)

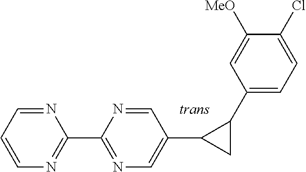 [SMILES: COc1cc(C2CC2c2cnc(-c3ncccn3)nc2)ccc1Cl]

### Example 34: trans-5-(2-(4-Chloro-3-methoxyphenyl)cyclopropyl)-2,2′-bipyrimidine (single enantiomer II)

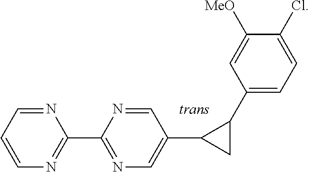 [SMILES: COc1cc(C2CC2c2cnc(-c3ncccn3)nc2)ccc1Cl]

A mixture of enantiomers (100 mg) was separated by SFC (supercritical fluid chromatography) on a CHIRALPAK® IG column using liquid CO₂ and MeOH [50:50; 0.1% methanolic NH₃ as modifier)] to give trans-5-(2-(4-chloro-3-methoxyphenyl)cyclopropyl)-2,2′-bipyrimidine (single enantiomer I) as an off-white solid (faster eluting enantiomer, 15 mg, 15%, m/z: 339 [M+H]⁺ observed), and trans-5-(2-(4-chloro-3-methoxyphenyl)cyclopropyl)-2,2′-bipyrimidine (single enantiomer II) as an off-white solid (slower eluting enantiomer, 16 mg, 16%, m/z: 339 [M+H]⁺ observed).

### Example 33: trans-5-(2-(4-Chloro-3-methoxyphenyl)cyclopropyl)-2,2′-bipyrimidine (single enantiomer I)

m/z: 339 [M+H]⁺ observed. ¹H NMR (400 MHz, DMSO-d₆): δ 8.99 (d, 2H), 8.85 (s, 2H), 7.62 (t, 1H), 7.33 (d, 1H), 7.01 (s, 1H), 6.83-6.80 (m, 1H), 3.87 (s, 3H), 2.56-2.53 (m, 1H), 2.43-2.38 (m, 1H), 1.78-1.66 (m, 2H).

### Example 34: trans-5-(2-(4-Chloro-3-methoxyphenyl)cyclopropyl)-2,2′-bipyrimidine (single enantiomer II)

m/z: 339 [M+H]⁺ observed. ¹H NMR (400 MHz, DMSO-d₆): δ 8.99 (d, 2H), 8.85 (s, 2H), 7.62 (t, 1H), 7.33 (d, 1H), 7.01 (s, 1H), 6.83-6.80 (m, 1H), 3.87 (s, 3H), 2.56-2.53 (m, 1H), 2.43-2.38 (m, 1H), 1.78-1.66 (m, 2H).

### Example 35: trans-5-(2-(2-Ethyl-4-fluoro-5-methoxyphenyl)cyclopropyl)-2,2′-bipyrimidine (single enantiomer I)

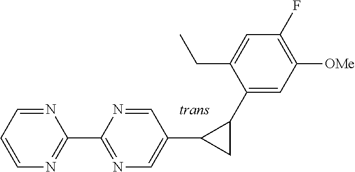 [SMILES: CCc1cc(F)c(OC)cc1C1CC1c1cnc(-c2ncccn2)nc1]

### Example 36: trans-5-(2-(2-Ethyl-4-fluoro-5-methoxyphenyl)cyclopropyl)-2,2′-bipyrimidine (single enantiomer II)

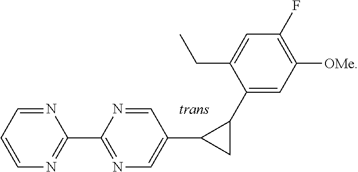 [SMILES: CCc1cc(F)c(OC)cc1C1CC1c1cnc(-c2ncccn2)nc1]

A mixture of enantiomers (30 mg) was separated by HPLC on a CHIRALPAK® IG column using n-hexane and EtOH (30:70) to give trans-5-(2-(2-ethyl-4-fluoro-5-methoxyphenyl)cyclopropyl)-2,2′-bipyrimidine (single enantiomer I) as an off-white solid (faster eluting enantiomer, 11 mg, 37%, m/z: 351 [M+H]⁺ observed), and trans-5-(2-(2-ethyl-4-fluoro-5-methoxyphenyl)cyclopropyl)-2,2′-bipyrimidine (single enantiomer II) as an off-white solid (slower eluting enantiomer, 9 mg, 30%, m/z: 351 [M+H]⁺ observed).

### Example 35: trans-5-(2-(2-Ethyl-4-fluoro-5-methoxyphenyl)cyclopropyl)-2,2′-bipyrimidine (single enantiomer I)

m/z: 351 [M+H]⁺ observed. ¹H NMR (400 MHz, DMSO-d₆): δ 8.99 (d, 2H), 8.90 (s, 2H), 7.64-7.61 (m, 1H), 7.05-7.01 (m, 1H), 6.89-6.86 (m, 1H), 3.85 (s, 3H), 2.67-2.58 (m, 2H), 2.51-2.50 (m, 1H), 2.25-2.20 (m, 1H), 1.78-1.68 (m, 2H), 1.10-1.06 (m, 3H).

### Example 36: trans-5-(2-(2-Ethyl-4-fluoro-5-methoxyphenyl)cyclopropyl)-2,2′-bipyrimidine (single enantiomer II)

m/z: 351 [M+H]⁺ observed. ¹H NMR (400 MHz, DMSO-d₆): δ 8.99 (d, 2H), 8.90 (s, 2H), 7.64-7.61 (m, 1H), 7.05-7.01 (m, 1H), 6.89-6.86 (m, 1H), 3.85 (s, 3H), 2.67-2.58 (m, 2H), 2.51-2.50 (m, 1H), 2.25-2.20 (m, 1H), 1.78-1.68 (m, 2H), 1.10-1.06 (m, 3H).

### Example 37: trans-5-(2-(4-Fluoro-5-methoxy-2-propylphenyl)cyclopropyl)-2,2′-bipyrimidine (single enantiomer I)

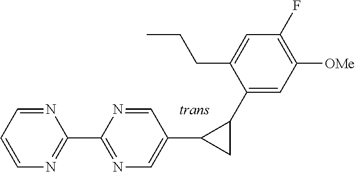 [SMILES: CCCc1cc(F)c(OC)cc1C1CC1c1cnc(-c2ncccn2)nc1]

### Example 38: trans-5-(2-(4-Fluoro-5-methoxy-2-propylphenyl)cyclopropyl)-2,2′-bipyrimidine (single enantiomer II)

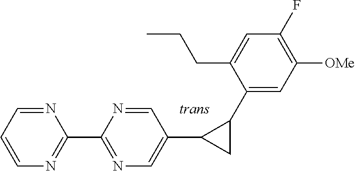 [SMILES: CCCc1cc(F)c(OC)cc1C1CC1c1cnc(-c2ncccn2)nc1]

A mixture of enantiomers (60 mg) was separated by SFC (supercritical fluid chromatography) on a CHIRALPAK® IG column using liquid CO₂ and MeOH [60:40; 0.1% methanolic NH₃ as modifier)] to give trans-5-(2-(4-fluoro-5-methoxy-2-propylphenyl)cyclopropyl)-2,2′-bipyrimidine (single enantiomer I) as a brown solid (faster eluting enantiomer, 19 mg, 37%, m/z: 365 [M+H]⁺ observed), and trans-5-(2-(4-fluoro-5-methoxy-2-propylphenyl)cyclopropyl)-2,2′-bipyrimidine (single enantiomer II) as a brown solid (slower eluting enantiomer, 27 mg, 30%, m/z: 365 [M+H]⁺ observed).

### Example 37: trans-5-(2-(4-Fluoro-5-methoxy-2-propylphenyl)cyclopropyl)-2,2′-bipyrimidine (single enantiomer I)

m/z: 365 [M+H]⁺ observed. ¹H NMR (400 MHz, DMSO-d₆): δ 8.99 (d, 2H), 8.90 (s, 2H), 7.64-7.61 (m, 1H), 7.03-7.00 (m, 1H), 6.87-6.85 (m, 1H), 3.86 (s, 3H), 2.58-2.50 (m, 3H), 2.23-2.20 (m, 1H), 1.77-1.73 (m, 1H), 1.69-1.65 (m, 1H), 1.50-1.45 (m, 2H), 0.76 (t, 3H).

### Example 38: trans-5-(2-(4-Fluoro-5-methoxy-2-propylphenyl)cyclopropyl)-2,2′-bipyrimidine (single enantiomer II)

m/z: 365 [M+H]⁺ observed. ¹H NMR (400 MHz, DMSO-d₆): δ 8.99 (d, 2H), 8.90 (s, 2H), 7.64-7.61 (m, 1H), 7.03-7.00 (m, 1H), 6.87-6.85 (m, 1H), 3.86 (s, 3H), 2.58-2.50 (m, 3H), 2.23-2.20 (m, 1H), 1.77-1.73 (m, 1H), 1.69-1.65 (m, 1H), 1.50-1.45 (m, 2H), 0.76 (t, 3H).

### Example 39: trans-5-(2-(4-Fluoro-3-(trifluoromethoxy)phenyl)cyclopropyl)-2,2′-bipyrimidine (single enantiomer I)

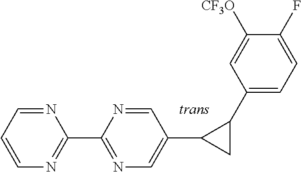

### Example 40: trans-5-(2-(4-Fluoro-3-(trifluoromethoxy)phenyl)cyclopropyl)-2,2′-bipyrimidine (single enantiomer II)

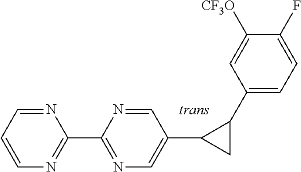

A mixture of enantiomers (70 mg) was separated by SFC (supercritical fluid chromatography) on a CHIRALPAK® IC column using liquid CO₂ and IPA [50:50; 0.1% methanolic NH₃ as modifier)] to give trans-5-(2-(4-fluoro-3-(trifluoromethoxy)phenyl)cyclopropyl)-2,2′-bipyrimidine (single enantiomer I) as a pale orange solid (faster eluting enantiomer, 15 mg, 21%, m/z: 377 [M+H]⁺ observed), and trans-5-(2-(4-fluoro-3-(trifluoromethoxy)phenyl)cyclopropyl)-2,2′-bipyrimidine (single enantiomer II) as a pale orange solid (slower eluting enantiomer, 15 mg, 21%, m/z: 377 [M+H]⁺ observed).

### Example 39: trans-5-(2-(4-Fluoro-3-(trifluoromethoxy)phenyl)cyclopropyl)-2,2′-bipyrimidine (single enantiomer I)

m/z: 377 [M+H]⁺ observed. ¹H NMR (400 MHz, DMSO-d₆): δ 8.99 (d, 2H), 8.85 (s, 2H), 7.63-7.61 (m, 1H), 7.48-7.43 (m, 2H), 7.35-7.31 (m, 1H), 2.65-2.61 (m, 1H), 2.43-2.38 (m, 1H), 1.79-1.74 (m, 1H), 1.70-1.65 (m, 1H).

### Example 40: trans-5-(2-(4-Fluoro-3-(trifluoromethoxy)phenyl)cyclopropyl)-2,2′-bipyrimidine (single enantiomer II)

m/z: 377 [M+H]⁺ observed. ¹H NMR (400 MHz, DMSO-d₆): δ 8.99 (d, 2H), 8.85 (s, 2H), 7.63-7.61 (m, 1H), 7.48-7.43 (m, 2H), 7.35-7.31 (m, 1H), 2.65-2.61 (m, 1H), 2.43-2.38 (m, 1H), 1.79-1.74 (m, 1H), 1.70-1.65 (m, 1H).

### Example 41: trans-5-(2-(4-Fluoro-3-(4-(methylsulfonyl)piperazin-1-yl)phenyl)cyclopropyl)-2,2′-bipyrimidine (single enantiomer I)

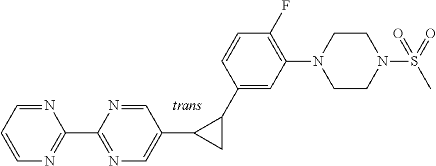 [SMILES: CS(=O)(=O)N1CCN(c2cc(C3CC3c3cnc(-c4ncccn4)nc3)ccc2F)CC1]

### Example 42: trans-5-(2-(4-Fluoro-3-(4-(methylsulfonyl)piperazin-1-yl)phenyl)cyclopropyl)-2,2′-bipyrimidine (single enantiomer II)

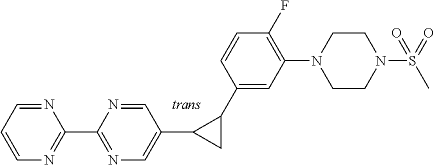 [SMILES: CS(=O)(=O)N1CCN(c2cc(C3CC3c3cnc(-c4ncccn4)nc3)ccc2F)CC1]

A mixture of enantiomers (190 mg) was separated by SFC (supercritical fluid chromatography) on a CHIRALPAK® OD-H column using liquid CO₂ and MeOH (50:50) to give trans-5-(2-(4-fluoro-3-(4-(methylsulfonyl)piperazin-1-yl)phenyl)cyclopropyl)-2,2′-bipyrimidine (single enantiomer I) as an off-white solid (faster eluting enantiomer, 49 mg, 26%, m/z: 455 [M+H]⁺ observed), and trans-5-(2-(4-fluoro-3-(4-(methylsulfonyl)piperazin-l-yl)phenyl)cyclopropyl)-2,2′-bipyrimidine (single enantiomer II) as an off-white solid (slower eluting enantiomer, 46 mg, 24%, m/z: 455 [M+H]⁺ observed).

### Example 41: trans-5-(2-(4-Fluoro-3-(4-(methylsulfonyl)piperazin-1-yl)phenyl)cyclopropyl)-2,2′-bipyrimidine (single enantiomer I)

m/z: 455 [M+H]⁺ observed. ¹H NMR (400 MHz, DMSO-d₆): δ 8.99 (d, 2H), 8.84 (s, 2H), 7.63-7.61 (m, 1H), 7.12-7.07 (m, 1H), 6.94-6.91 (m, 1H), 6.87-6.84 (m, 1H), 3.28-3.26 (m, 4H), 3.14-3.11 (m, 4H), 2.93 (s, 3H), 2.53-2.49 (m, 1H), 2.36-2.33 (m, 1H), 1.72-1.68 (m, 1H), 1.66-1.62 (m, 1H).

### Example 42: trans-5-(2-(4-Fluoro-3-(4-(methylsulfonyl)piperazin-1-yl)phenyl)cyclopropyl)-2,2′-bipyrimidine (single enantiomer II)

m/z: 455 [M+H]⁺ observed. ¹H NMR (400 MHz, DMSO-d₆): δ 8.99 (d, 2H), 8.84 (s, 2H), 7.63-7.61 (m, 1H), 7.12-7.07 (m, 1H), 6.94-6.91 (m, 1H), 6.87-6.84 (m, 1H), 3.28-3.26 (m, 4H), 3.14-3.11 (m, 4H), 2.93 (s, 3H), 2.53-2.49 (m, 1H), 2.36-2.33 (m, 1H), 1.72-1.68 (m, 1H), 1.66-1.62 (m, 1H).

### Example 43: trans-5-(2-(3-(3,3-Difluoropyrrolidin-1-yl)-4-fluorophenyl)cyclopropyl)-2,2′-bipyrimidine (single enantiomer I)

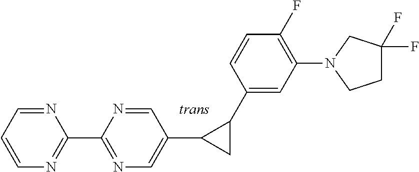 [SMILES: Fc1ccc(C2CC2c2cnc(-c3ncccn3)nc2)cc1N1CCC(F)(F)C1]

### Example 44: trans-5-(2-(3-(3,3-Difluoropyrrolidin-1-yl)-4-fluorophenyl)cyclopropyl)-2,2′-bipyrimidine (single enantiomer II)

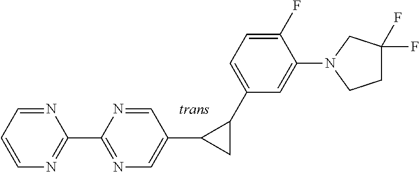 [SMILES: Fc1ccc(C2CC2c2cnc(-c3ncccn3)nc2)cc1N1CCC(F)(F)C1]

A mixture of enantiomers of trans-5-(2-(3-(3,3-difluoropyrrolidin-1-yl)-4-fluorophenyl)cyclopropyl)-2,2′-bipyrimidine (100 mg) was separated by SFC (supercritical fluid chromatography) on a CHIRALPAK® IC column using liquid CO₂ and 30 mM methanolic ammonia in EtOH (50:50) to give trans-5-(2-(3-(3,3-difluoropyrrolidin-1-yl)-4-fluorophenyl)cyclopropyl)-2,2′-bipyrimidine (single enantiomer I) as an off-white solid (faster eluting enantiomer, 12 mg, 12%, m/z: 398 [M+H]⁺ observed), and trans-5-(2-(3-(3,3-difluoropyrrolidin-1-yl)-4-fluorophenyl)cyclopropyl)-2,2′-bipyrimidine (single enantiomer II) as an off-white solid (slower eluting enantiomer, 20 mg, 20%, m/z: 398 [M+H]⁺ observed).

### Example 43: trans-5-(2-(3-(3,3-Difluoropyrrolidin-1-yl)-4-fluorophenyl)cyclopropyl)-2,2′-bipyrimidine (single enantiomer I)

m/z: 398 [M+H]⁺ observed. ¹H NMR (400 MHz, DMSO-d₆): δ 8.98 (d, 2H), 8.83 (s, 2H), 7.62 (t, 1H), 7.07-7.02 (m, 1H), 6.68-6.65 (m, 2H), 3.73 (t, 2H), 3.53 (t, 2H), 2.50-2.43 (m, 3H), 2.35-2.31 (m, 1H), 1.70-1.62 (m, 2H).

### Example 44: trans-5-(2-(3-(3,3-Difluoropyrrolidin-1-yl)-4-fluorophenyl)cyclopropyl)-2,2′-bipyrimidine (single enantiomer II)

m/z: 398 [M+H]⁺ observed. ¹H NMR (400 MHz, DMSO-d₆): δ 8.98 (d, 2H), 8.83 (s, 2H), 7.62 (t, 1H), 7.07-7.02 (m, 1H), 6.68-6.65 (m, 2H), 3.73 (t, 2H), 3.53 (t, 2H), 2.50-2.43 (m, 3H), 2.35-2.31 (m, 1H), 1.70-1.62 (m, 2H).

### Example 45: trans-5-(2-(4-Chloro-3-fluoro-5-methoxyphenyl)cyclopropyl)-2,2′-bipyrimidine (single enantiomer I)

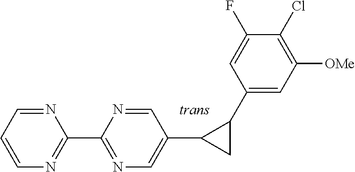 [SMILES: COc1cc(C2CC2c2cnc(-c3ncccn3)nc2)cc(F)c1Cl]

### Example 46: trans-5-(2-(4-Chloro-3-fluoro-5-methoxyphenyl)cyclopropyl)-2,2′-bipyrimidine (single enantiomer II)

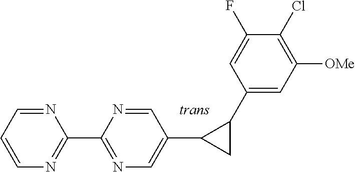 [SMILES: COc1cc(C2CC2c2cnc(-c3ncccn3)nc2)cc(F)c1Cl]

A mixture of enantiomers of trans-5-(2-(4-chloro-3-fluoro-5-methoxyphenyl)cyclopropyl)-2,2′-bipyrimidine (80 mg) was separated by SFC (supercritical fluid chromatography) on a CHIRALCEL® OD-H column using liquid CO₂ and MeOH (50:50) to give trans-5-(2-(4-chloro-3-fluoro-5-methoxyphenyl)cyclopropyl)-2,2′-bipyrimidine (single enantiomer I) as a brick red solid (faster eluting enantiomer, 23 mg, 29%, m/z: 357 [M+H]⁺ observed), and trans-5-(2-(3-(3,3-difluoropyrrolidin-1-yl)-4-fluorophenyl)cyclopropyl)-2,2′-bipyrimidine (single enantiomer II) as a pale orange solid (slower eluting enantiomer, 31 mg, 39%, m/z: 357 [M+H]⁺ observed).

### Example 45: trans-5-(2-(4-Chloro-3-fluoro-5-methoxyphenyl)cyclopropyl)-2,2′-bipyrimidine (single enantiomer I)

m/z: 357 [M+H]⁺ observed. ¹H NMR (400 MHz, DMSO-d₆): δ 8.99 (d, 2H), 8.85 (s, 2H), 7.63 (t, 1H), 6.92-6.88 (m, 2H), 3.90 (s, 3H), 2.59-2.54 (m, 1H), 2.46-2.43 (m, 1H), 1.81-1.71 (m, 2H).

### Example 46: trans-5-(2-(4-Chloro-3-fluoro-5-methoxyphenyl)cyclopropyl)-2,2′-bipyrimidine (single enantiomer II)

m/z: 357 [M+H]⁺ observed. ¹H NMR (400 MHz, DMSO-d₆): δ 8.99 (d, 2H), 8.85 (s, 2H), 7.63 (t, 1H), 6.92-6.88 (m, 2H), 3.90 (s, 3H), 2.59-2.54 (m, 1H), 2.46-2.43 (m, 1H), 1.81-1.71 (m, 2H).

### Example 47: trans-5-(2-(4-Fluoro-3-(3-methoxyazetidin-1-yl)phenyl)cyclopropyl)-2,2′-bipyrimidine (single enantiomer I)

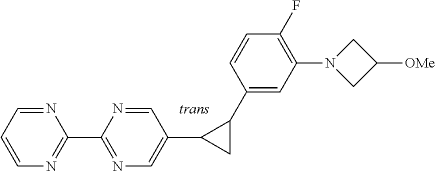 [SMILES: COC1CN(c2cc(C3CC3c3cnc(-c4ncccn4)nc3)ccc2F)C1]

### Example 48: trans-5-(2-(4-Fluoro-3-(3-methoxyazetidin-1-yl)phenyl)cyclopropyl)-2,2′-bipyrimidine (single enantiomer II)

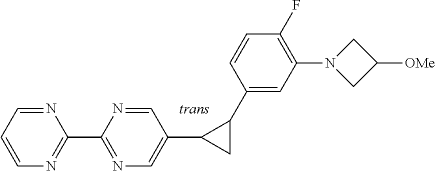 [SMILES: COC1CN(c2cc(C3CC3c3cnc(-c4ncccn4)nc3)ccc2F)C1]

A mixture of enantiomers of trans-5-(2-(4-fluoro-3-(3-methoxyazetidin-1-yl)phenyl)cyclopropyl)-2,2′-bipyrimidine (130 mg) was separated by SFC (supercritical fluid chromatography) on a CHIRALCEL® OD-H column using liquid CO₂ and MeOH (50:50) to give trans-5-(2-(4-fluoro-3-(3-methoxyazetidin-1-yl)phenyl)cyclopropyl)-2,2′-bipyrimidine (single enantiomer I) as a pale yellow solid (faster eluting enantiomer, 43 mg, 33%, m/z: 378 [M+H]⁺ observed), and trans-5-(2-(4-fluoro-3-(3-methoxyazetidin-1-yl)phenyl)cyclopropyl)-2,2′-bipyrimidine (single enantiomer II) as a pale yellow solid (slower eluting enantiomer, 36 mg, 27%, m/z: 378 [M+H]⁺ observed).

### Example 47: trans-5-(2-(4-Fluoro-3-(3-methoxyazetidin-1-yl)phenyl)cyclopropyl)-2,2′-bipyrimidine (single enantiomer I)

m/z: 378 [M+H]⁺ observed. ¹H NMR (400 MHz, DMSO-d₆): δ 8.99 (d, 2H), 8.83 (s, 2H), 7.62 (t, 1H), 6.99-6.94 (m, 1H), 6.58-6.55 (m, 1H), 6.41-6.38 (m, 1H), 4.28-4.26 (m, 1H), 4.15-4.10 (m, 2H), 3.69-3.66 (m, 2H), 3.23 (s, 3H), 2.49-2.43 (m, 1H), 2.32-2.29 (m, 1H), 1.68-1.66 (m, 1H), 1.60-1.58 (m, 1H).

### Example 48: trans-5-(2-(4-Fluoro-3-(3-methoxyazetidin-1-yl)phenyl)cyclopropyl)-2,2′-bipyrimidine (single enantiomer II)

m/z: 378 [M+H]⁺ observed. ¹H NMR (400 MHz, DMSO-d₆): δ 8.99 (d, 2H), 8.83 (s, 2H), 7.62 (t, 1H), 6.99-6.94 (m, 1H), 6.58-6.55 (m, 1H), 6.41-6.38 (m, 1H), 4.28-4.26 (m, 1H), 4.15-4.10 (m, 2H), 3.69-3.66 (m, 2H), 3.23 (s, 3H), 2.49-2.43 (m, 1H), 2.32-2.29 (m, 1H), 1.68-1.66 (m, 1H), 1.60-1.58 (m, 1H).

### Example 49: trans-5-((2-(4-Chloro-3-(cyclopropylmethoxy)-5-methylphenyl)cyclopropyl)-2,2′-bipyrimidine (single enantiomer I)

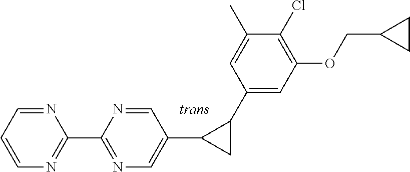 [SMILES: Cc1cc(C2CC2c2cnc(-c3ncccn3)nc2)cc(OCC2CC2)c1Cl]

### Example 50: trans-5-((2-(4-Chloro-3-(cyclopropylmethoxy)-5-m ethylphenyl)cyclopropyl)-2,2′-bipyrimidine (single enantiomer II)

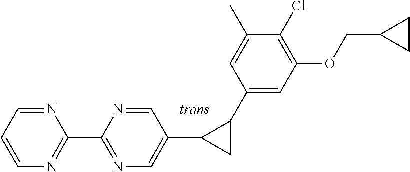 [SMILES: Cc1cc(C2CC2c2cnc(-c3ncccn3)nc2)cc(OCC2CC2)c1Cl]

A mixture of enantiomers of trans-5-((2-(4-chloro-3-(cyclopropylmethoxy)-5-methylphenyl)cyclopropyl)-2,2′-bipyrimidine (110 mg) was separated by SFC (supercritical fluid chromatography) on a CHIRALPAK® AD column using liquid CO₂ and IPA (0.1% aqueous NH₃) (50:50) to give trans-5-((2-(4-chloro-3-(cyclopropylmethoxy)-5-methylphenyl)cyclopropyl)-2,2′-bipyrimidine (single enantiomer I) as a yellow solid (faster eluting enantiomer, 31 mg, 28% yield, m/z: 393 [M+H]⁺ observed) and trans-5-((2-(4-chloro-3-(cyclopropylmethoxy)-5-methylphenyl)cyclopropyl)-2,2′-bipyrimidine (single enantiomer II) as a brown solid (slower eluting enantiomer, 32 mg, 29% yield, m/z: 393 [M+H]⁺ observed).

### Example 49: trans-5-((2-(4-Chloro-3-(cyclopropylmethoxy)-5-methylphenyl)cyclopropyl)-2,2′-bipyrimidine (single enantiomer I)

m/z: 393 [M+H]⁺ observed. ¹H NMR (400 MHz, DMSO-d₆): δ 8.94 (d, J=4.8 Hz, 2 H), 8.83 (s, 2 H), 7.62 (t, J=4.8 Hz, 1 H), 6.80 (d, J=6.8 Hz, 2 H), 3.91 (d, J=4.8 Hz, 2 H), 2.50-2.45 (m, 1 H), 2.37-2.33 (m, 1 H), 2.29 (s, 3 H), 1.73-1.65 (m, 2 H), 1.27-1.23 (m, 1H), 0.60-0.57 (m, 2H), 0.36-0.33 (m, 2H).

### Example 50: trans-5-((2-(4-Chloro-3-(cyclopropylmethoxy)-5-methylphenyl)cyclopropyl)-2,2′-bipyrimidine (single enantiomer II)

m/z: 393 [M+H]⁺ observed. ¹H NMR (400 MHz, DMSO-d₆): δ 8.94 (d, J=4.8 Hz, 2 H), 8.83 (s, 2 H), 7.62 (t, J=4.8 Hz, 1 H), 6.80 (d, J=6.8 Hz, 2 H), 3.91 (d, J=4.8 Hz, 2 H), 2.50-2.45 (m, 1 H), 2.37-2.33 (m, 1 H), 2.29 (s, 3 H), 1.73-1.65 (m, 2 H), 1.27-1.23 (m, 1H), 0.60-0.57 (m, 2H), 0.36-0.33 (m, 2H).

### Example 51: trans-5-(2-(4-Chloro-5-methoxy-2-methylphenyl)cyclopropyl)-2,2′-bipyrimidine (single enantiomer I)

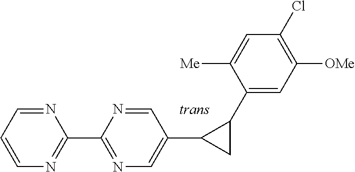 [SMILES: COc1cc(C2CC2c2cnc(-c3ncccn3)nc2)c(C)cc1Cl]

### Example 52: trans-5-(2-(4-Chloro-5-methoxy-2-methylphenyl)cyclopropyl)-2,2′-bipyrimidine (single enantiomer II)

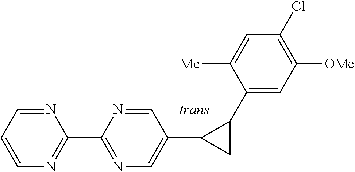 [SMILES: COc1cc(C2CC2c2cnc(-c3ncccn3)nc2)c(C)cc1Cl]

A mixture of enantiomers of trans-5-(2-(4-chloro-5-methoxy-2-methylphenyl)cyclopropyl)-2,2′-bipyrimidine (53 mg) was separated by SFC (supercritical fluid chromatography) on a CHIRALPAK® AD-H column using liquid CO₂ and 30 mM Methanolic ammonia in EtOH (50:50) to give trans-5-(2-(4-chloro-5-methoxy-2-methylphenyl)cyclopropyl)-2,2′-bipyrimidine (single enantiomer I) as a brown solid (faster eluting enantiomer, 8 mg, 15%, m/z: 353 [M+H]⁺ observed), and trans-5-(2-(4-chloro-5-methoxy-2-methylphenyl)cyclopropyl)-2,2′-bipyrimidine (single enantiomer II) as a brown solid (slower eluting enantiomer, 16 mg, 30%, m/z: 353 [M+H]⁺ observed).

### Example 51: trans-5-(2-(4-Chloro-5-methoxy-2-methylphenyl)cyclopropyl)-2,2′-bipyrimidine (single enantiomer I)

m/z: 353 [M+H]⁺ observed. ¹H NMR (400 MHz, DMSO-d₆): δ 8.99 (d, 2H), 8.91 (s, 2H), 7.63 (t, 1H), 7.24 (s, 1H), 6.86 (s, 1H), 3.87 (s, 3H), 2.52-2.48 (m, 1H), 2.28-2.22 (m, 4H), 1.75-1.67 (m, 2H).

### Example 52: trans-5-(2-(4-Chloro-5-methoxy-2-methylphenyl)cyclopropyl)-2,2′-bipyrimidine (single enantiomer II)

m/z: 353 [M+H]⁺ observed. ¹H NMR (400 MHz, DMSO-d₆): δ 8.99 (d, 2H), 8.91 (s, 2H), 7.63 (t, 1H), 7.24 (s, 1H), 6.86 (s, 1H), 3.87 (s, 3H), 2.52-2.48 (m, 1H), 2.28-2.22 (m, 4H), 1.75-1.67 (m, 2H).

### Example 53: trans-1-(5-(2-([2,2′-Bipyrimidin]-5-yl)cyclopropyl)-2-fluorophenyl)azetidin-3-ol (single enantiomer I)

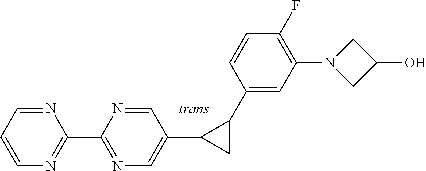 [SMILES: OC1CN(c2cc(C3CC3c3cnc(-c4ncccn4)nc3)ccc2F)C1]

### Example 54: trans-1-(5-(2-([2,2′-Bipyrimidin]-5-yl)cyclopropyl)-2-fluorophenyl)azetidin-3-ol (single enantiomer II)

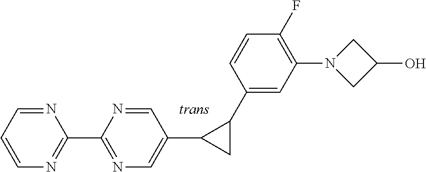 [SMILES: OC1CN(c2cc(C3CC3c3cnc(-c4ncccn4)nc3)ccc2F)C1]

A mixture of enantiomers of trans-1-(5-(2-([2,2′-bipyrimidin]-5-yl)cyclopropyl)-2-fluorophenyl)azetidin-3-ol (120 mg) was separated by SFC (supercritical fluid chromatography) on a CHIRALPAK® OD-H column using liquid CO₂ and MeOH (50:50) to give trans-1-(5-(2-([2,2′-bipyrimidin]-5-yl)cyclopropyl)-2-fluorophenyl)azetidin-3-ol (single enantiomer I) as a pale yellow solid (faster eluting enantiomer, 21 mg, 17%, m/z: 364 [M+H]⁺ observed), and trans-1-(5-(2-([2,2′-bipyrimidin]-5-yl)cyclopropyl)-2-fluorophenyl)azetidin-3-ol (single enantiomer II) as a pale yellow solid (slower eluting enantiomer, 23 mg, 19%, m/z: 364 [M+H]⁺ observed).

### Example 53: trans-1-(5-(2-([2,2′-Bipyrimidin]-5-yl)cyclopropyl)-2-fluorophenyl)azetidin-3-ol (single enantiomer I)

m/z: 364 [M+H]⁺ observed. ¹H NMR (400 MHz, DMSO-d₆): δ 8.99 (d, 2H), 8.83 (s, 2H), 7.62 (t, 1H), 6.98-6.93 (m, 1H), 6.56-6.53 (m, 1H), 6.40-6.38 (m, 1H), 5.57 (d, 1H), 4.53-4.52 (br s, 1H), 4.15-4.11 (m, 2H), 3.60-3.57 (m, 2H), 2.49-2.42 (m, 1H), 2.32-2.29 (m, 1H), 1.68-1.64 (m, 1H), 1.59-1.58 (m, 1H).

### Example 54: trans-1-(5-(2-([2,2′-Bipyrimidin]-5-yl)cyclopropyl)-2-fluorophenyl)azetidin-3-ol (single enantiomer II)

m/z: 364 [M+H]⁺ observed. ¹H NMR (400 MHz, DMSO-d₆): δ 8.99 (d, 2H), 8.83 (s, 2H), 7.62 (t, 1H), 6.98-6.93 (m, 1H), 6.56-6.53 (m, 1H), 6.40-6.38 (m, 1H), 5.57 (d, 1H), 4.53-4.52 (br s, 1H), 4.15-4.11 (m, 2H), 3.60-3.57 (m, 2H), 2.49-2.42 (m, 1H), 2.32-2.29 (m, 1H), 1.68-1.64 (m, 1H), 1.59-1.58 (m, 1H).

### Example 55: trans-5-(2-(4-Chloro-2-fluoro-3-methoxyphenyl)cyclopropyl)-2,2′-bipyrimidine (single enantiomer I)

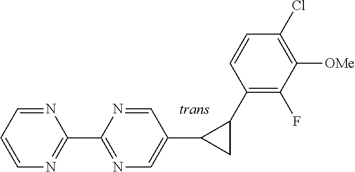 [SMILES: COc1c(Cl)ccc(C2CC2c2cnc(-c3ncccn3)nc2)c1F]

### Example 56: trans-5-(2-(4-Chloro-2-fluoro-3-methoxyphenyl)cyclopropyl)-2,2′-bipyrimidine (single enantiomer II)

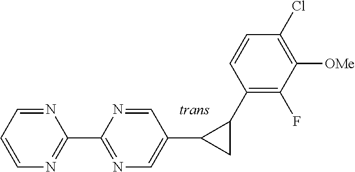 [SMILES: COc1c(Cl)ccc(C2CC2c2cnc(-c3ncccn3)nc2)c1F]

A mixture of enantiomers of trans-5-(2-(4-chloro-2-fluoro-3-methoxyphenyl)cyclopropyl)-2,2′-bipyrimidine (110 mg) was separated by chiral chromatography on a CHIRALPAK® IC column using n-Hexane:Ethanol (20:80) to give trans-5-(2-(4-chloro-2-fluoro-3-methoxyphenyl)cyclopropyl)-2,2′-bipyrimidine (single enantiomer I) as a pale gray solid (faster eluting enantiomer, 9 mg, 8%, m/z: 357 [M+H]⁺ observed), and trans-5-(2-(4-chloro-2-fluoro-3-methoxyphenyl)cyclopropyl)-2,2′-bipyrimidine (single enantiomer II) as a pale brown solid (slower eluting enantiomer, 14 mg, 12%, m/z: 357 [M+H]⁺ observed).

### Example 55: trans-5-(2-(4-Chloro-2-fluoro-3-methoxyphenyl)cyclopropyl)-2,2′-bipyrimidine (single enantiomer I)

m/z: 357 [M+H]⁺ observed. ¹H NMR (400 MHz, DMSO-d₆): δ 8.99 (d, 2H), 8.89 (s, 2H), 7.62 (t, 1H), 7.30-7.27 (d, 1H), 7.03-6.99 (m, 1H), 3.88(s, 3H), 2.59-2.50 (m, 1H), 2.49-2.41 (m, 1H), 1.80-1.66 (m, 2H).

### Example 56: trans-5-(2-(4-Chloro-2-fluoro-3-methoxyphenyl)cyclopropyl)-2,2′-bipyrimidine (single enantiomer II)

m/z: 357 [M+H]⁺ observed. ¹H NMR (400 MHz, DMSO-d₆): δ 8.99 (d, 2H), 8.89 (s, 2H), 7.62 (t, 1H), 7.30-7.27 (d, 1H), 7.03-6.99 (m, 1H), 3.88(s, 3H), 2.59-2.50 (m, 1H), 2.49-2.41 (m, 1H), 1.80-1.66 (m, 2H).

### Example 57: trans-5-(2-(4-Chloro-3-methoxy-5-methylphenyl)cyclopropyl)-2,2′-bipyrimidine (single enantiomer I)

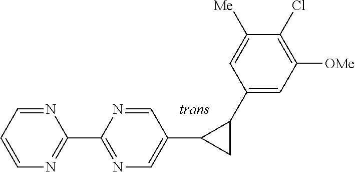 [SMILES: COc1cc(C2CC2c2cnc(-c3ncccn3)nc2)cc(C)c1Cl]

### Example 58: trans-5-(2-(4-Chloro-3-methoxy-5-methylphenyl)cyclopropyl)-2,2′-bipyrimidine (single enantiomer II)

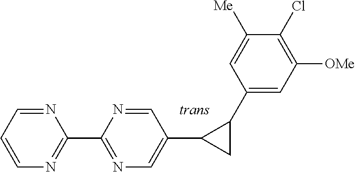 [SMILES: COc1cc(C2CC2c2cnc(-c3ncccn3)nc2)cc(C)c1Cl]

A mixture of enantiomers of trans-5-(2-(4-chloro-3-methoxy-5-methylphenyl)cyclopropyl)-2,2′-bipyrimidine (200 mg) was separated by SFC (supercritical fluid chromatography) on a LUX® Amylose-2 column using liquid CO₂ and MeOH (50:50) to give trans-5-(2-(4-chloro-3-methoxy-5-methylphenyl)cyclopropyl)-2,2′-bipyrimidine (single enantiomer I) as an off-white solid (faster eluting enantiomer, 35 mg, 17%, m/z: 353 [M+H]⁺ observed), and trans-5-(2-(4-chloro-3-methoxy-5-methylphenyl)cyclopropyl)-2,2′-bipyrimidine (single enantiomer II) as an off-white solid (slower eluting enantiomer, 35 mg, 17%, m/z: 353 [M+H]⁺ observed).

### Example 57: trans-5-(2-(4-Chloro-3-methoxy-5-methylphenyl)cyclopropyl)-2,2′-bipyrimidine (single enantiomer I)

m/z: 353 [M+H]⁺ observed. ¹H NMR (400 MHz, DMSO-d₆): δ 8.99 (d, 2H), 8.84 (s, 2H), 7.62 (t, 1H), 6.86-6.81 (m, 2H), 3.85(s, 3H), 2.50-2.46 (m, 1H), 2.39-2.37 (m, 1H), 2.30 (s, 3H), 1.75-1.68 (m, 2H).

### Example 58: trans-5-(2-(4-Chloro-3-methoxy-5-methylphenyl)cyclopropyl)-2,2′-bipyrimidine (single enantiomer II)

m/z: 353 [M+H]⁺ observed. ¹H NMR (400 MHz, DMSO-d₆): δ 8.99 (d, 2H), 8.84 (s, 2H), 7.62 (t, 1H), 6.86-6.81 (m, 2H), 3.85(s, 3H), 2.50-2.46 (m, 1H), 2.39-2.37 (m, 1H), 2.30 (s, 3H), 1.75-1.68 (m, 2H).

### Example 59: trans-5-(2-(3,4-Dichloro-5-methoxyphenyl)cyclopropyl)-2,2′-bipyrimidine (single enantiomer I)

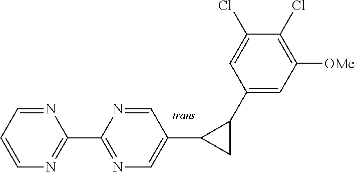 [SMILES: COc1cc(C2CC2c2cnc(-c3ncccn3)nc2)cc(Cl)c1Cl]

### Example 60: trans-5-(2-(3,4-Dichloro-5-methoxyphenyl)cyclopropyl)-2,2′-bipyrimidine (single enantiomer II)

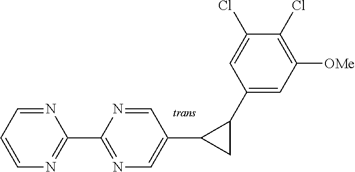 [SMILES: COc1cc(C2CC2c2cnc(-c3ncccn3)nc2)cc(Cl)c1Cl]

A mixture of enantiomers of trans-5-(2-(3,4-dichloro-5-methoxyphenyl)cyclopropyl)-2,2′-bipyrimidine (160 mg) was separated by SFC (supercritical fluid chromatography) on a LUX® Amylose-2 column using liquid CO₂ and 30 mM Methanolic ammonia in MeOH (60:40) to give trans-5-(2-(3,4-dichloro-5-methoxyphenyl)cyclopropyl)-2,2′-bipyrimidine (single enantiomer I) as an off-white solid (faster eluting enantiomer, 35 mg, 21%, m/z: 373 [M+H]⁺ observed), and trans-5-(2-(3,4-dichloro-5-methoxyphenyl)cyclopropyl)-2,2′-bipyrimidine (single enantiomer II) as an off-white solid (slower eluting enantiomer, 41 mg, 25%, m/z: 373 [M+H]⁺ observed).

### Example 59: trans-5-(2-(3,4-Dichloro-5-methoxyphenyl)cyclopropyl)-2,2′-bipyrimidine (single enantiomer I)

m/z: 373 [M+H]⁺ observed. ¹H NMR (400 MHz, DMSO-d₆): δ 8.99 (d, 2H), 8.85 (s, 2H), 7.62 (t, 1H), 7.10 (s, 1H), 7.02 (s, 1H), 3.90 (s, 3H), 2.57-2.54 (m, 1H), 2.47-2.44 (m, 1H), 1.79-1.74 (m, 2H).

### Example 60: trans-5-(2-(3,4-Dichloro-5-methoxyphenyl)cyclopropyl)-2,2′-bipyrimidine (single enantiomer II)

m/z: 373 [M+H]⁺ observed. ¹H NMR (400 MHz, DMSO-d₆): δ 8.99 (d, 2H), 8.85 (s, 2H), 7.62 (t, 1H), 7.10 (s, 1H), 7.02 (s, 1H), 3.90 (s, 3H), 2.57-2.54 (m, 1H), 2.47-2.44 (m, 1H), 1.79-1.74 (m, 2H).

### Example 61: trans-5-(2-(4-Chloro-3-(cyclopropylmethoxy)-2-methylphenyl)cyclopropyl)-2,2′-bipyrimidine (single enantiomer I)

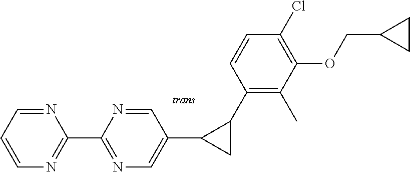 [SMILES: Cc1c(C2CC2c2cnc(-c3ncccn3)nc2)ccc(Cl)c1OCC1CC1]

### Example 62: trans-5-(2-(4-Chloro-3-(cyclopropylmethoxy)-2-methylphenyl)cyclopropyl)-2,2′-bipyrimidine (single enantiomer II)

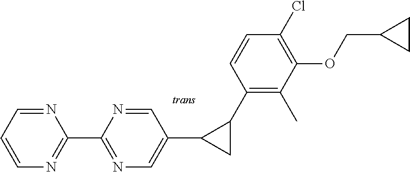 [SMILES: Cc1c(C2CC2c2cnc(-c3ncccn3)nc2)ccc(Cl)c1OCC1CC1]

A mixture of enantiomers of trans-5-(2-(4-chloro-3-(cyclopropylmethoxy)-2-methylphenyl)cyclopropyl)-2,2′-bipyrimidine (95 mg) was separated by SFC (supercritical fluid chromatography) on a CHIRALCEL® OD column using liquid CO₂ and EtOH (0.1% aqueous NH₃) (45:55) to give trans-5-(2-(4-chloro-3-(cyclopropylmethoxy)-2-methylphenyl)cyclopropyl)-2,2′-bipyrimidine (single enantiomer I) as an off-white solid (faster eluting enantiomer, 29 mg, 31% yield, m/z: 393 [M+H]⁺ observed) and trans-5-(2-(4-chloro-3-(cyclopropylmethoxy)-2-methylphenyl)cyclopropyl)-2,2′-bipyrimidine (single enantiomer II) as a yellow solid (slower eluting enantiomer, 33 mg, 35% yield, m/z: 393 [M+H]⁺ observed).

### Example 61: trans-5-(2-(4-Chloro-3-(cyclopropylmethoxy)-2-methylphenyl)cyclopropyl)-2,2′-bipyrimidine (single enantiomer I)

m/z: 393 [M+H]⁺ observed. ¹H NMR (400 MHz, DMSO-d₆): δ 9.00 (d, J=4.8 Hz, 2H), 8.91 (s, 2H), 7.63 (t, J=4.8 Hz, 1H), 7.26 (d, J=8.4 Hz, 1H), 6.98 (d, J=8.4 Hz, 1H), 3.69 (d, J=6.8 Hz, 2H), 2.27 (s, 3H), 2.23-2.19 (m, 2H), 1.72-1.68 (m, 1H), 1.65-1.61 (m, 1H), 1.28-1.20 (m, 1H), 0.58-0.54 (m, 2H), 0.34-0.28 (m, 2H).

### Example 62: trans-5-(2-(4-Chloro-3-(cyclopropylmethoxy)-2-methylphenyl)cyclopropyl)-2,2′-bipyrimidine (single enantiomer II)

m/z: 393 [M+H]⁺ observed. ¹H NMR (400 MHz, DMSO-d₆): δ 9.00 (d, J=4.8 Hz, 2H), 8.91 (s, 2H), 7.63 (t, J=4.8 Hz, 1H), 7.26 (d, J=8.4 Hz, 1H), 6.98 (d, J=8.4 Hz, 1H), 3.69 (d, J=6.8 Hz, 2H), 2.27 (s, 3H), 2.23-2.19 (m, 2H), 1.72-1.68 (m, 1H), 1.65-1.61 (m, 1H), 1.28-1.20 (m, 1H), 0.58-0.54 (m, 2H), 0.34-0.28 (m, 2H).

### Example 63: trans-5-(2-([2,2′-Bipyrimidin]-5-yl)cyclopropyl)-2-fluoro-N,N-dimethylaniline (single enantiomer I)

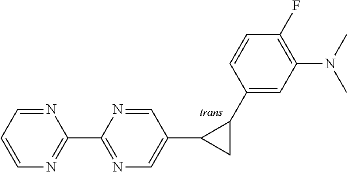 [SMILES: CN(C)c1cc(C2CC2c2cnc(-c3ncccn3)nc2)ccc1F]

### Example 64: trans-5-(2-([2,2′-Bipyrimidin]-5-yl)cyclopropyl)-2-fluoro-N,N-dimethylaniline (single enantiomer II)

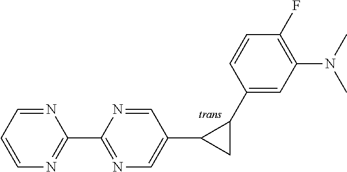 [SMILES: CN(C)c1cc(C2CC2c2cnc(-c3ncccn3)nc2)ccc1F]

A mixture of enantiomers of trans-5-(2-([2,2′-bipyrimidin]-5-yl)cyclopropyl)-2-fluoro-N,N-dimethylaniline (200 mg) was separated by SFC (supercritical fluid chromatography) on a CHIRALCEL® AD column using liquid CO₂ and IPA (45%; 0.1% aqueous NH₃ as modifier) to give trans-5-(2-([2,2′-bipyrimidin]-5-yl)cyclopropyl)-2-fluoro-N,N-dimethylaniline (single enantiomer I) as a yellow solid (faster eluting enantiomer, 103 mg, 49% yield, m/z: 336 [M+H]⁺ observed) and trans-5-(2-([2,2′-bipyrimidin]-5-yl)cyclopropyl)-2-fluoro-N,N-dimethylaniline (trans, single enantiomer II) as a yellow solid (slower eluting enantiomer, 103 mg, 49% yield, m/z: 336 [M+H]⁺ observed).

### Example 63: trans-5-(2-([2,2′-Bipyrimidin]-5-yl)cyclopropyl)-2-fluoro-N,N-dimethylaniline (single enantiomer I)

m/z: 336 [M+H]⁺ observed. ¹H NMR (400 MHz, DMSO-d₆): δ 8.99 (d, J=4.8 Hz, 2H), 8.84 (s, 2H), 7.62 (t, J=4.8 Hz, 1H), 7.05-7.00 (m, 1H), 6.83-6.80 (m, 1H), 6.73-6.69 (m, 1H), 2.78 (s, 6H), 2.50-2.47 (m, 1H), 2.36-2.31 (m, 1H), 1.72-1.60 (m, 2H).

### Example 64: trans-5-(2-([2,2′-Bipyrimidin]-5-yl)cyclopropyl)-2-fluoro-N,N-dimethylaniline (single enantiomer II)

m/z: 336 [M+H]⁺ observed. ¹H NMR (400 MHz, DMSO-d₆): δ 8.99 (d, J=4.8 Hz, 2H), 8.84 (s, 2H), 7.62 (t, J=4.8 Hz, 1H), 7.05-7.00 (m, 1H), 6.83-6.80 (m, 1H), 6.73-6.69 (m, 1H), 2.78 (s, 6H), 2.50-2.47 (m, 1H), 2.36-2.31 (m, 1H), 1.72-1.60 (m, 2H).

### Example 65: trans-(3S)-1-(5-(2-([2,2′-Bipyrimidin]-5-yl)cyclopropyl)-2-fluorophenyl)pyrrolidin-3-ol (single diastereomer I)

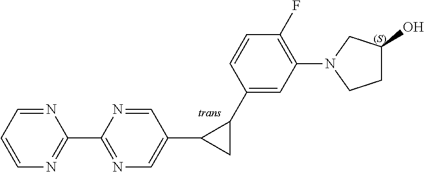

### Example 66: trans-(3S)-1-(5-(2-([2,2′-Bipyrimidin]-5-yl)cyclopropyl)-2-fluorophenyl)pyrrolidin-3-ol (single diastereomer II)

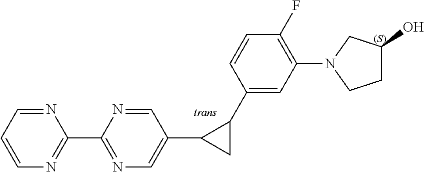

A mixture of diastereomers of trans-(3S)-1-(5-(2-([2,2′-bipyrimidin]-5-yl)cyclopropyl)-2-fluorophenyl)pyrrolidin-3-ol (60 mg) was separated by SFC (supercritical fluid chromatography) on a CHIRALCEL® OD-H column using liquid CO₂ and MeOH (50:50) to give trans-(3S)-1-(5-(2-([2,2′-bipyrimidin]-5-yl)cyclopropyl)-2-fluorophenyl)pyrrolidin-3-ol (single diastereomer I) as an off-white solid (faster eluting diastereomer, 12 mg, 20%, m/z: 378 [M+H]⁺ observed), and trans-(3S)-1-(5-(2-([2,2′-bipyrimidin]-5-yl)cyclopropyl)-2-fluorophenyl)pyrrolidin-3-ol (single diastereomer II) as an off-white solid (slower eluting diastereomer, 14 mg, 23%, m/z: 378 [M+H]⁺ observed).

### Example 65: trans-(3S)-1-(5-(2-([2,2′-Bipyrimidin]-5-yl)cyclopropyl)-2-fluorophenyl)pyrrolidin-3-ol (single diastereomer I)

m/z: 378 [M+H]⁺ observed. ¹H NMR (400 MHz, DMSO-d₆): δ 8.99 (d, 2H), 8.83 (s, 2H), 7.62 (t, 1H), 6.99-6.93 (m, 1H), 6.55-6.53 (m, 1H), 6.49-6.46 (m, 1H), 4.90 (s, 1H), 4.33 (s, 1H), 3.57-3.54 (m, 1H), 3.46-3.44 (m, 1H), 3.33-3.30 (m, 1H), 3.17-3.14 (m, 1H), 2.46-2.44 (m, 1H), 2.33-2.30 (m, 1H), 1.98-1.94 (m, 1H), 1.83-1.82 (m, 1H), 1.68-1.66 (m, 1H), 1.59-1.57 (m, 1H).

### Example 66: trans-(3S)-1-(5-(2-([2,2′-Bipyrimidin]-5-yl)cyclopropyl)-2-fluorophenyl)pyrrolidin-3-ol (single diastereomer II)

m/z: 378 [M+H]⁺ observed. ¹H NMR (400 MHz, DMSO-d₆): δ 8.99 (d, 2H), 8.83 (s, 2H), 7.62 (t, 1H), 6.99-6.93 (m, 1H), 6.55-6.53 (m, 1H), 6.49-6.46 (m, 1H), 4.90 (s, 1H), 4.33 (s, 1H), 3.57-3.54 (m, 1H), 3.46-3.44 (m, 1H), 3.33-3.30 (m, 1H), 3.17-3.14 (m, 1H), 2.46-2.44 (m, 1H), 2.33-2.30 (m, 1H), 1.98-1.94 (m, 1H), 1.83-1.82 (m, 1H), 1.68-1.66 (m, 1H), 1.59-1.57 (m, 1H).

### Example 67: trans-5-(2-(4-Fluoro-3-((S)-3-methoxypyrrolidin-1-yl)phenyl)cyclopropyl)-2,2′-bipyrimidine (single diastereomer I)

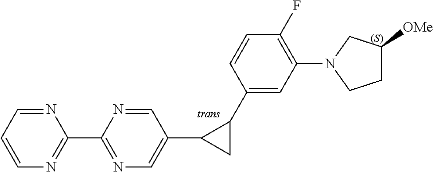

### Example 68: trans-5-(2-(4-Fluoro-3-((S)-3-methoxypyrrolidin-1-yl)phenyl)cyclopropyl)-2,2′-bipyrimidine (single diastereomer II)

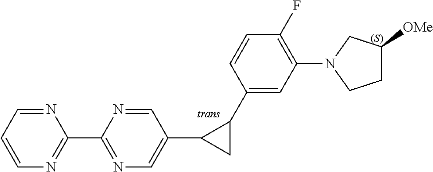

A mixture of diasteromers of trans-5-(2-(4-fluoro-3-((S)-3-methoxypyrrolidin-1-yl)phenyl)cyclopropyl)-2,2′-bipyrimidine (350 mg) was separated by SFC (supercritical fluid chromatography) on a CHIRALCEL® OD-H column using liquid CO₂ and MeOH (50:50) to give trans-5-(2-(4-fluoro-3-((S)-3-methoxypyrrolidin-1-yl)phenyl)cyclopropyl)-2,2′-bipyrimidine (single diastereomer I) as a pale yellow solid (faster eluting diastereomer, 60 mg, 17%, m/z: 392 [M+H]⁺ observed), and trans-5-(2-(4-fluoro-3-((S)-3-methoxypyrrolidin-1-yl)phenyl)cyclopropyl)-2,2′-bipyrimidine (single diastereomer II) as an off-white solid (slower eluting diastereomer, 62 mg, 18%, m/z: 392 [M+H]⁺ observed).

### Example 67: trans-5-(2-(4-Fluoro-3-((-3-methoxypyrrolidin-1-yl)phenyl)cyclopropyl)-2,2′-bipyrimidine (single diastereomer I)

m/z: 392 [M+H]⁺ observed. ¹H NMR (400 MHz, DMSO-d₆): δ 8.98 (d, 2H), 8.83 (s, 2H), 7.62 (t, 1H), 7.00-6.94 (m, 1H), 6.58-6.56 (m, 1H), 6.52-6.50 (m, 1H), 4.03-4.02 (m, 1H), 3.54-3.52 (m, 1H), 3.40-3.27 (m, 3H), 3.25 (s, 3H), 2.49-2.44 (m, 1H), 2.32-2.30 (m, 1H), 2.00-1.97 (m, 2H), 1.68-1.66 (m, 1H), 1.60-1.58 (m, 1H).

### Example 68: trans-5-(2-(4-Fluoro-34(S)-3-methoxypyrrolidin-1-yl)phenyl)cyclopropyl)-2,2′-bipyrimidine (single diastereomer II)

m/z: 392 [M+H]⁺ observed. ¹H NMR (400 MHz, DMSO-d₆): δ 8.98 (d, 2H), 8.83 (s, 2H), 7.62 (t, 1H), 7.00-6.94 (m, 1H), 6.58-6.56 (m, 1H), 6.52-6.50 (m, 1H), 4.03-4.02 (m, 1H), 3.54-3.52 (m, 1H), 3.40-3.27 (m, 3H), 3.25 (s, 3H), 2.49-2.44 (m, 1H), 2.32-2.30 (m, 1H), 2.00-1.97 (m, 2H), 1.68-1.66 (m, 1H), 1.60-1.58 (m, 1H).

### Example 69: trans-(3R)-1-(5-(2-([2,2′-Bipyrimidin]-5-yl)cyclopropyl)-2-fluorophenyl)pyrrolidin-3-ol (single diastereomer I)

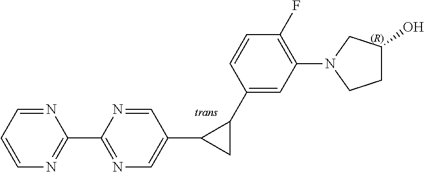

### Example 70: trans-(3R)-1-(5-(2-([2,2′-Bipyrimidin]-5-yl)cyclopropyl)-2-fluorophenyl)pyrrolidin-3-ol (single diastereomer II)

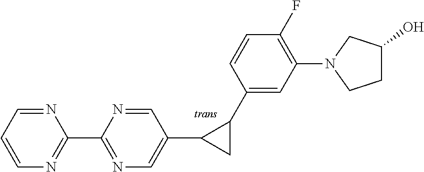 [SMILES: O[C@@H]1CCN(c2cc(C3CC3c3cnc(-c4ncccn4)nc3)ccc2F)C1]

A mixture of diastereomers of trans-(3R)-1-(5-(2-([2,2′-bipyrimidin]-5-yl)cyclopropyl)-2-fluorophenyl)pyrrolidin-3-ol (180 mg) was separated by SFC (supercritical fluid chromatography) on a CHIRALCEL® OD-H column using liquid CO₂ and MeOH (50:50) to give trans-(3R)-1-(5-(2-([2,2′-bipyrimidin]-5-yl)cyclopropyl)-2-fluorophenyl)pyrrolidin-3-ol (single diastereomer I) as an off-white solid (faster eluting diastereomer, 44 mg, 24%, m/z: 378 [M+H]⁺ observed), and trans-(3R)-1-(5-(2-([2,2′-bipyrimidin]-5-yl)cyclopropyl)-2-fluorophenyl)pyrrolidin-3-ol (single diastereomer II) as an off-white solid (slower eluting diastereomer, 14 mg, 7%, m/z: 378 [M+H]⁺ observed).

### Example 69: trans-(3R)-1-(5-(2-([2,2′-Bipyrimidin]-5-yl)cyclopropyl)-2-fluorophenyl)pyrrolidin-3-ol (single diastereomer I)

m/z: 378 [M+H]⁺ observed. ¹H NMR (400 MHz, DMSO-d₆): δ 8.99 (d, 2H), 8.83 (s, 2H), 7.62 (t, 1H), 6.99-6.93 (m, 1H), 6.55-6.53 (m, 1H), 6.49-6.46 (m, 1H), 4.90 (d, 1H), 4.33 (s, 1H), 3.57-3.54 (m, 1H), 3.46-3.44 (m, 1H), 3.33-25 (m, 1H), 3.17-3.14 (m, 1H), 2.46-2.44 (m, 1H), 2.33-2.29 (m, 1H), 1.98-1.94 (m, 1H), 1.83-1.82 (m, 1H), 1.68-1.66 (m, 1H), 1.59-1.57 (m, 1H).

### Example 70: trans-(3R)-1-(5-(2-([2,2′-Bipyrimidin]-5-yl)cyclopropyl)-2-fluorophenyl)pyrrolidin-3-ol (single diastereomer II)

m/z: 378 [M+H]⁺ observed. ¹H NMR (400 MHz, DMSO-d₆): δ 8.99 (d, 2H), 8.83 (s, 2H), 7.62 (t, 1H), 6.99-6.93 (m, 1H), 6.55-6.53 (m, 1H), 6.49-6.46 (m, 1H), 4.90 (d, 1H), 4.33 (s, 1H), 3.57-3.54 (m, 1H), 3.46-3.44 (m, 1H), 3.33-25 (m, 1H), 3.17-3.14 (m, 1H), 2.46-2.44 (m, 1H), 2.33-2.29 (m, 1H), 1.98-1.94 (m, 1H), 1.83-1.82 (m, 1H), 1.68-1.66 (m, 1H), 1.59-1.57 (m, 1H).

### Example 71: trans-5-(2-(4-Chloro-3-methoxy-2-methylphenyl)cyclopropyl)-2,2′-bipyrimidine (single enantiomer I)

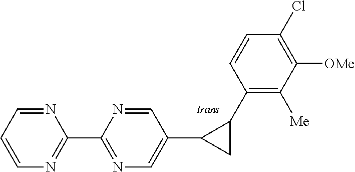 [SMILES: COc1c(Cl)ccc(C2CC2c2cnc(-c3ncccn3)nc2)c1C]

### Example 72: trans-5-(2-(4-Chloro-3-methoxy-2-methylphenyl)cyclopropyl)-2,2′-bipyrimidine (single enantiomer II)

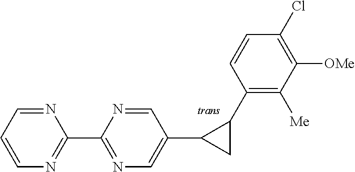 [SMILES: COc1c(Cl)ccc(C2CC2c2cnc(-c3ncccn3)nc2)c1C]

A mixture of enantiomers of trans-5-(2-(4-chloro-3-methoxy-2-methylphenyl)cyclopropyl)-2,2′-bipyrimidine (100 mg) was separated by SFC (supercritical fluid chromatography) on a CHIRALCEL® OD-H column using liquid CO₂ and MeOH (60:40) to give trans-5-(2-(4-chloro-3-methoxy-2-methylphenyl)cyclopropyl)-2,2′-bipyrimidine (single enantiomer I) as an off-white solid (faster eluting enantiomer, 30 mg, 30%, m/z: 353 [M+H]⁺ observed), and trans-5-(2-(4-chloro-3-methoxy-2-methylphenyl)cyclopropyl)-2,2′-bipyrimidine (single enantiomer II) as an off-white solid (slower eluting enantiomer, 30 mg, 30%, m/z: 353 [M+H]⁺ observed).

### Example 71: trans-5-(2-(4-Chloro-3-methoxy-2-methylphenyl)cyclopropyl)-2,2′-bipyrimidine (single enantiomer I)

m/z: 353 [M+H]⁺ observed. ¹H NMR (400 MHz, DMSO-d₆): δ 9.00 (d, 2H), 8.91 (s, 2H), 7.62 (t, 1H), 7.28 (d, 1H), 7.00 (d, 1H), 3.72 (s, 3H), 2.50-2.47 (m, 1H), 2.26 (s, 3H), 2.25-2.19 (m, 1H), 1.72-1.60 (m, 2H).

### Example 72: trans-5-(2-(4-Chloro-3-methoxy-2-methylphenyl)cyclopropyl)-2,2′-bipyrimidine (single enantiomer II)

m/z: 353 [M+H]⁺ observed. ¹H NMR (400 MHz, DMSO-d₆): δ 9.00 (d, 2H), 8.91 (s, 2H), 7.62 (t, 1H), 7.28 (d, 1H), 7.00 (d, 1H), 3.72 (s, 3H), 2.50-2.47 (m, 1H), 2.26 (s, 3H), 2.25-2.19 (m, 1H), 1.72-1.60 (m, 2H).

### Example 73: cis-5-(2-(2-Fluoro-4-methoxyphenyl)cyclopropyl)-2,2′-bipyrimidine

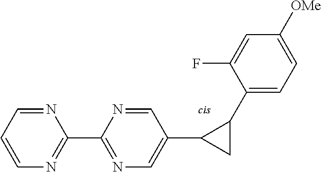 [SMILES: COc1ccc(C2CC2c2cnc(-c3ncccn3)nc2)c(F)c1]

### 1-Ethynyl-2-fluoro-4-methoxybenzene

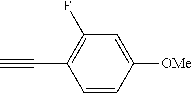

A solution of 1-bromo-2-fluoro-4-methoxybenzene (5.0 g, 24.4 mmol) in triethylamine (50 mL) was treated with ethynyltrimethylsilane (10.2 mL, 73.3 mmol) and degassed with argon for 5 minutes. To the reaction mixture was added CuI (47 mg, 0.24 mmol), followed by bis(triphenylphosphine)palladium(II) dichloride (171 mg, 0.24 mmol) at rt, and the reaction mixture was heated at 100° C. for 16 h. The reaction mixture was cooled to rt and evaporated under reduced pressure. The obtained residue was dissolved in MeOH-CH₂Cl₂ (1:1, 100 mL), K₂CO₃ (13.5 g, 97.6 mmol) was added, and the mixture was stirred at rt for 16 h. The reaction mixture was filtered, and the filtrate was evaporated. The residue was purified by normal phase SiO₂ chromatography (0-3% EtOAc/petroleum ether) to give 1-ethynyl-2-fluoro-4-methoxybenzene as a brown liquid (1.5g, 41% yield). ¹H NMR (400 MHz, CDCl₃): δ 7.38 (t, 1H), 6.68-6.60 (m, 2H), 3.81 (s, 3H), 3.21 (s, 1H).

### 2-Chloro-5-((2-fluoro-4-methoxyphenyl)ethynyl)pyrimidine

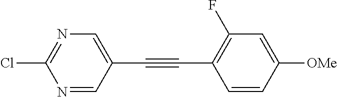 [SMILES: COc1ccc(C#Cc2cnc(Cl)nc2)c(F)c1]

To a solution of 1-ethynyl-2-fluoro-4-methoxybenzene (1.0 g, 6.7 mmol) and 5-bromo-2-chloro pyrimidine (1.3g, 6.7 mmol) in THF (10 mL) was added triethylamine (2.6 mL, 18.6 mmol), and the mixture was degassed with argon for 5 min in a sealed tube. Bis(triphenylphosphine) palladium(II) dichloride (234 mg, 0.33 mmol) was added and the mixture was degassed with argon for 2 minutes. The reaction mixture was heated at 100° C. for 16 h. The reaction mixture was cooled to rt, diluted with EtOAc (50 mL) and filtered through a CELITE® pad. The filtrate was washed with saturated aqueous brine solution (20 mL), dried over anhydrous sodium sulfate, filtered, and evaporated under reduced pressure. The residue was purified by normal phase SiO₂ chromatography (0-5% EtOAc/petroleum ether) to give 2-chloro-5-((2-fluoro-4-methoxyphenyl)ethynyl)pyrimidine as a yellow solid (600 mg, 34% yield, m/z: 263 [M+H]⁺ observed). ¹H NMR (400 MHz, CDCl₃): δ 8.72 (s, 2H), 7.43 (t, 1H), 6.73-6.61 (m, 2H), 3.84 (s, 3H).

### (Z)-2-Chloro-5-(2-fluoro-4-methoxystyryl)pyrimidine:

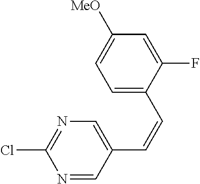 [SMILES: COc1ccc(/C=C\c2cnc(Cl)nc2)c(F)c1]

To a solution of 2-chloro-5-((2-fluoro-4-methoxyphenyl)ethynyl)pyrimidine (450 mg, 1.7 mmol) in EtOAc (10 mL) was added Pd-CaCO₃ (5 wt. % loading on calcium carbonate, 225 mg, 0.11 mmol) at rt and stirred under hydrogen balloon pressure for 2 h. The reaction mixture was filtered through a CELITE® pad and was evaporated under reduced pressure.

The residue was purified by normal phase SiO₂ chromatography (0-10% EtOAc/petroleum ether) to give (Z)-2-chloro-5-(2-fluoro-4-methoxystyryl)pyrimidine as a pale yellow solid (280 mg, 61% yield, m/z: 265 [M+H]⁺ observed). ¹H NMR (400 MHz, CDCl₃): δ 8.44 (s, 2H), 7.04 (t, 1H), 6.81 (d, 1H), 6.63-6.59 (m, 2H), 6.45 (d, 1H), 3.79 (s, 3H). cis-2-Chloro-5-(2-(2-fluoro-4-methoxyphenyl)cyclopropyl)pyrimidine:

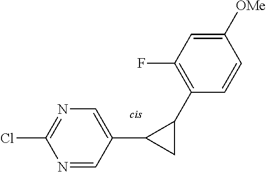 [SMILES: COc1ccc(C2CC2c2cnc(Cl)nc2)c(F)c1]

To a solution of (Z)-2-chloro-5-(2-fluoro-4-methoxystyryl)pyrimidine (280 mg, 1.06 mmol) in CH₂Cl₂ (2 mL) at 0° C. was added Pd₃(OAc)₆ (71 mg, 0.10 mmol) and ethereal diazomethane [freshly prepared from N-methyl-N-nitroso urea (2.2 g, 21.2 mmol), KOH (50% aqueous solution, 20 mL) and Et₂O (20 mL) at 0° C.] and stirred at 0° C. for 20 h. The reaction mixture was filtered through a CELITE® pad and the filtrate was evaporated. The residue was dissolved again in CH₂Cl₂ (2 mL), followed by the addition of Pd₃(OAc)₆ (71 mg, 0.10 mmol) and ethereal diazomethane [freshly prepared from N-methyl-N-nitroso urea (2.2 g, 21.2 mmol), KOH (50% aqueous solution, 20 mL) and Et₂O (20 mL) at 0° C.] and the mixture was stirred at 0° C. for 20 h. The reaction mixture was filtered through a CELITE® pad and the filtrate was evaporated. The residue was dissolved one more time in CH₂Cl₂ (2 mL), followed by the addition of Pd₃(OAc)₆ (71 mg, 0.10 mmol) and ethereal diazomethane [freshly prepared from N-methyl-N-nitroso urea (2.2 g, 21.2 mmol), KOH (50% aqueous solution, 20 mL) and Et₂O (20 mL) at 0° C.] and the mixture was stirred at 0° C. for 20 h. The reaction mixture was filtered through a CELITE® pad and filtrate was evaporated. The residue was purified by normal phase SiO₂ chromatography (0-10% EtOAc/petroleum ether) to give cis-2-chloro-5-(2-(2-fluoro-4-methoxyphenyl)cyclopropyl)pyrimidine as a pale yellow liquid (60 mg, 20% yield, m/z: 279 [M+H]⁺ observed). ¹H NMR (400 MHz, CDCl₃): δ 8.13 (s, 2H), 6.91 (t, 1H), 6.55-6.52 (m, 1H), 6.46-6.42 (m, 1H), 3.72 (s, 3H), 2.58-2.56 (m, 1H), 2.37-2.36 (m, 1H), 1.74-1.68 (m, 1H), 1.61-1.59 (m, 1H).

### cis-5-(2-(2-Fluoro-4-methoxyphenyl)cyclopropyl)-2,2′-bipyrimidine

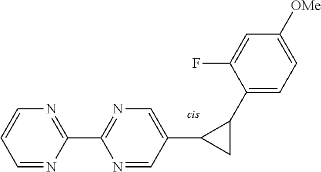 [SMILES: COc1ccc(C2CC2c2cnc(-c3ncccn3)nc2)c(F)c1]

To a solution of cis-2-chloro-5-(2-(2-fluoro-4-methoxyphenyl)cyclopropyl)pyrimidine (190 mg, 0.68 mmol) in DMF (2 mL) was added 2-(tributylstannyl)pyrimidine (0.21 mL, 0.65 mmol), tetraethylammonium chloride (107 mg, 0.65 mmol) and K₂CO₃ (178 mg, 1.29 mmol) at rt and the mixture was degassed with N₂ gas for 10 min. Bis(triphenylphosphine)palladium(II) dichloride (45 mg, 0.064 mmol) was added and the mixture was purged with N₂ gas for 10 min. The reaction mixture was stirred at 100° C. for 16 h, diluted with water (20 mL), and extracted with EtOAc (2×20 mL). The combined organic layer was washed with saturated aqueous brine solution (20 mL), dried over anhydrous sodium sulfate, filtered, and evaporated under reduced pressure. The residue was purified by reverse phase HPLC to give cis-5-(2-(2-fluoro-4-methoxyphenyl)cyclopropyl)-2,2′-bipyrimidine as an off-white solid (9.6 mg, 4% yield, m/z: 323 [M+H]⁺ observed). ¹H NMR (400 MHz, DMSO-d₆): δ 8.93-8.92 (m, 2H), 8.52 (s, 2H), 7.57 (t, 1H), 7.23-7.19 (m, 1H), 6.66-6.59 (m, 2H) 3.66 (s, 3H), 2.63-2.59 (m, 2H), 1.87-1.85 (m, 1H), 1.47-1.45 (m, 1H).

### Example 74: trans-5-(2-(4-Fluoro-3-methoxyphenyl)cyclopropyl)-4-isopropyl-2,2′-bipyrimidine (single enantiomer I)

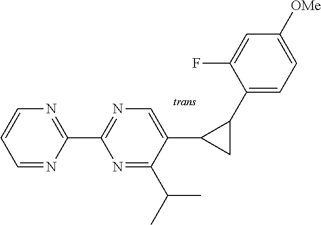 [SMILES: COc1ccc(C2CC2c2cnc(-c3ncccn3)nc2C(C)C)c(F)c1]

### Example 75: trans-5-(2-(4-Fluoro-3-methoxyphenyl)cyclopropyl)-4-isopropyl-2,2′-bipyrimidine (single enantiomer II)

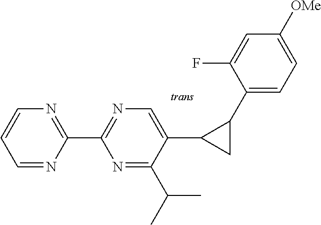 [SMILES: COc1ccc(C2CC2c2cnc(-c3ncccn3)nc2C(C)C)c(F)c1]

### 5-Bromo-2-chloro-4-isopropylpyrimidine

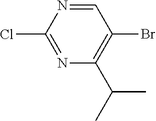

To a solution of 5-bromo-2-chloropyrimidine (5.0 g, 25.8 mmol) in CH₂Cl₂/H₂O (1:1, 40 mL) was added isobutyric acid (3.8 mL, 41 mmol), AgNO₃ (3.5 g, 20.7 mmol), and K₂S₂O₈ (11.2 g, 41.4 mmol). The reaction mixture was stirred at room temperature for 16 h. The reaction mixture was diluted with water (200 mL) and extracted with CH₂Cl₂ (2×200 mL). The combined organic phase was washed with saturated aqueous brine solution (200 mL), dried over anhydrous sodium sulfate, filtered, and evaporated under reduced pressure. The residue was purified by normal phase SiO₂ chromatography (0-5% EtOAc/petroleum ether) to give 5-bromo-2-chloro-4-isopropylpyrimidine as a white solid (1.6 g, 26% yield, m/z: 233 [M+H]⁺ observed). ¹H NMR (400 MHz, CDCl₃): δ 8.57 (s, 1H), 3.41-3.47 (m, 1H), 1.26-1.60 (m, 6H).

### (E)-2-Chloro-5-(4-fluoro-3-methoxystyryl)-4-isopropylpyrimidine

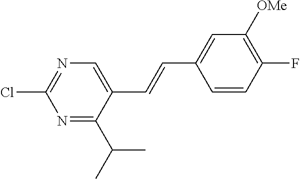 [SMILES: COc1cc(/C=C/c2cnc(Cl)nc2C(C)C)ccc1F]

To a solution of 5-bromo-2-chloro-4-isopropylpyrimidine (2.0 g, 8.5 mmol) in acetonitrile (10 mL) was added 1-fluoro-2-methoxy-4-vinylbenzene (2.0 g, 13 mmol) and DIPEA (3 mL, 17 mmol) at room temperature, and the mixture was degassed with N₂ gas for 10 min. Then Pd(OAc)₂ (0.19 g, 0.85 mmol) was added and the mixture was purged with N₂ gas for 10 min. The reaction mixture was heated at 100° C. for 16 h in a sealed tube. The reaction mixture was diluted with water (200 mL) and extracted with EtOAc (2×200 mL). The combined organic phase was washed with saturated aqueous brine solution (200 mL), dried over anhydrous sodium sulfate, filtered, and evaporated under reduced pressure. The residue was purified by normal phase SiO₂ chromatography (0-20% EtOAc/petroleum ether) to give (E)-2-chloro-5-(4-fluoro-3-methoxystyryl)-4-isopropylpyrimidine as a white solid (0.50 g, 19% yield, m/z: 307 [M+H]⁺ observed).

### trans-2-Chloro-5-(2-(4-fluoro-3-methoxyphenyl)cyclopropyl)-4-isopropylpyrimidine

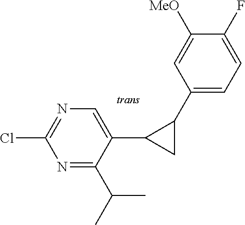 [SMILES: COc1cc(C2CC2c2cnc(Cl)nc2C(C)C)ccc1F]

To a solution of (E)-2-chloro-5-(4-fluoro-3-methoxystyryl)-4-isopropylpyrimidine (0.50 g, 1.6 mmol) in CH₂Cl₂ (5 mL) at 0° C. was added Pd(OAc)₂ (36 mg, 0.16 mmol) and diazomethane [freshly prepared from of N-methyl-N-nitroso urea (1.68 g, 16.3 mmol), KOH (50% aqueous solution, 40 mL) of Et₂O (40 mL) at 0° C.] and stirred at 0-5° C. for 16 h. The reaction mixture was filtered through CELITE° pad and concentrated under reduced pressure. The residue was dissolved in CH₂Cl₂ (5 mL) at 0° C. and Pd(OAc)₂ (36 mg, 0.16 mmol) was added, followed by diazomethane [freshly prepared from of N-methyl-N-nitroso urea (1.68 g, 16.3 mmol), KOH (50% aqueous solution, 40 mL) of Et₂O (40 mL) at 0° C.]. The reaction mixture was stirred at 0-5° C. for 16 h. The residue was purified by normal phase SiO₂ chromatography (0-20% EtOAc/petroleum ether) to give trans-2-chloro-5-(2-(4-fluoro-3-methoxyphenyl)cyclopropyl)-4-isopropylpyrimidine as a yellow oil (0.20 g, 38% yield, m/z: 321 [M+H]⁺ observed). ¹H NMR (300 MHz, DMSO-d₆): δ 8.49 (s, 1H), 7.15-7.08 (m, 1H), 7.03-6.99 (m, 1H), 6.82-6.77 (m, 1H), 3.84 (s, 3H), 3.45-3.41 (m, 1H), 2.28-2.23 (m, 2H), 1.63-1.49 (m, 2H), 1.20-1.14 (m, 6H).

### trans-5-(2-(4-Fluoro-3-methoxyphenyl)cyclopropyl)-4-isopropyl-2,2′-bipyrimidine

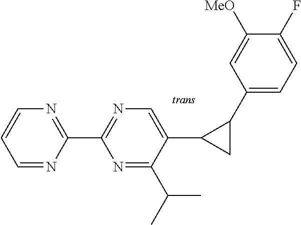 [SMILES: COc1cc(C2CC2c2cnc(-c3ncccn3)nc2C(C)C)ccc1F]

To a solution of trans-2-chloro-5-(2-(4-fluoro-3-methoxyphenyl)cyclopropyl)-4-isopropylpyrimidine (0.30 g, 0.94 mmol) in DMF (5 mL) was added 2-(tributylstannyl)pyrimidine (0.45 mL, 1.4 mmol), tetraethylammonium chloride (0.16 g, 0.97 mmol), and K₂CO₃ (0.26 g, 1.9 mmol) at rt, and the mixture purged with N₂ gas for 10 min. Bis(triphenylphosphine)palladium(II) dichloride (66 mg, 0.094 mmol) was added, and the mixture was purged with N₂ gas for 10 min. The reaction mixture was stirred at 110° C. for 16 h. The reaction mixture was diluted with water (100 mL) and extracted with EtOAc (2×200 mL). The combined organic phase was washed with saturated aqueous brine solution (100 mL), dried over anhydrous sulfate, filtered and evaporated under reduced pressure. The residue was purified by reverse phase HPLC to give trans-5-(2-(4-fluoro-3-methoxyphenyl)cyclopropyl)-4-isopropyl-2,2′-bipyrimidine as an off-white solid (50.0 mg, 14% yield, m/z: 365 [M+H]⁺ observed). ¹H NMR (400 MHz, DMSO-d₆): δ 8.99 (d, 2H), 8.67 (s, 1H), 7.63-7.61 (m, 1H), 7.13-7.06 (m, 1H), 7.06-7.04 (m, 1H), 6.85-6.81 (m, 1H), 3.85 (s, 3H), 2.54-2.47 (m, 1H), 2.39-2.30 (m, 2H), 1.70-1.68 (m, 1H), 1.57-1.54 (m, 1H), 1.26-1.21 (m, 6H).

A mixture of enantiomers (50 mg) was separated by SFC (supercritical fluid chromatography) on a CHIRALPAK® IG column using liquid CO₂ and EtOH (60:40) to give trans-5-(2-(4-fluoro-3-methoxyphenyl)cyclopropyl)-4-isopropyl-2,2′-bipyrimidine (single enantiomer I) as a white solid (faster eluting enantiomer, 17 mg, 34%, m/z: 365 [M+H]⁺ observed), and trans-5-(2-(4-fluoro-3-methoxyphenyl)cyclopropyl)-4-isopropyl-2,2′-bipyrimidine (single enantiomer II) as an off-white solid (slower eluting enantiomer, 18 mg, 36%, m/z: 365 [M+H]⁺ observed).

### Example 74: trans-5-(2-(4-Fluoro-3-methoxyphenyl)cyclopropyl)-4-isopropyl-2,2′-bipyrimidine (single enantiomer I)

m/z: 365 [M+H]⁺ observed. ¹H NMR (400 MHz, DMSO-d₆): δ 8.99 (d, 2H), 8.67 (s, 1H), 7.63-7.61 (m, 1H), 7.13-7.06 (m, 1H), 7.06-7.04 (m, 1H), 6.85-6.81 (m, 1H), 3.85 (s, 3H), 2.54-2.47 (m, 1H), 2.39-2.30 (m, 2H), 1.70-1.68 (m, 1H), 1.57-1.54 (m, 1H), 1.26-1.21 (m, 6H).

### Example 75: trans-5-(2-(4-Fluoro-3-methoxyphenyl)cyclopropyl)-4-isopropyl-2,2′-bipyrimidine (single enantiomer II)

m/z: 365 [M+H]⁺ observed. ¹H NMR (400 MHz, DMSO-d₆): δ 8.99 (d, 2H), 8.67 (s, 1H), 7.63-7.61 (m, 1H), 7.13-7.06 (m, 1H), 7.06-7.04 (m, 1H), 6.85-6.81 (m, 1H), 3.85 (s, 3H), 2.54-2.47 (m, 1H), 2.39-2.30 (m, 2H), 1.70-1.68 (m, 1H), 1.57-1.54 (m, 1H), 1.26-1.21 (m, 6H).

The following examples were prepared in a similar manner as trans-5-(2-(4-fluoro-3-methoxyphenyl)cyclopropyl)-4-isopropyl-2,2′-bipyrimidine from an appropriately substituted styrene and an appropriately substituted 2-chloropyrimidine:

### Example 76: trans-4-Ethyl-5-(2-(4-fluoro-3-methoxyphenyl)cyclopropyl)-2,2′-bipyrimidine (single enantiomer I)

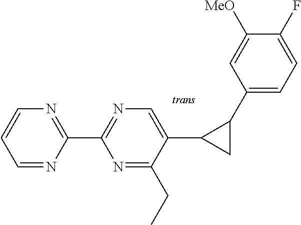 [SMILES: CCc1nc(-c2ncccn2)ncc1C1CC1c1ccc(F)c(OC)c1]

### Example 77: trans-4-Ethyl-5-(2-(4-fluoro-3-methoxyphenyl)cyclopropyl)-2,2′-bipyrimidine (single enantiomer II)

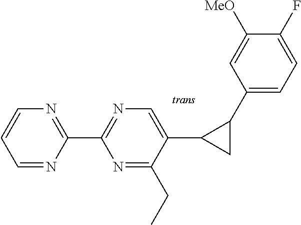 [SMILES: CCc1nc(-c2ncccn2)ncc1C1CC1c1ccc(F)c(OC)c1]

A mixture of enantiomers (35 mg) was separated by SFC (supercritical fluid chromatography) on a CHIRALPAK® IG column using liquid CO₂ and EtOH [60:40; 0.1% methanolic NH₃ as modifier)] to give trans-4-ethyl-5-(2-(4-fluoro-3-methoxyphenyl)cyclopropyl)-2,2′-bipyrimidine (single enantiomer I) as a white solid (faster eluting enantiomer, 9.5 mg, 27%, m/z: 351 [M+H]⁺ observed) and trans-4-ethyl-5-(2-(4-fluoro-3-methoxyphenyl)cyclopropyl)-2,2′-bipyrimidine (single enantiomer II) as an off-white solid (slower eluting enantiomer, 10 mg, 39%, m/z: 351 [M+H]⁺ observed).

### Example 76: trans-4-Ethyl-5-(2-(4-fluoro-3-methoxyphenyl)cyclopropyl)-2,2′-bipyrimidine (single enantiomer I)

m/z: 351 [M+H]⁺ observed. ¹H NMR (400 MHz, DMSO-d₆): δ 8.99 (d, 2H), 8.65 (s, 1H), 7.63-7.61 (m, 1H), 7.16-7.11 (m, 1H), 7.07-7.05 (m, 1H), 6.85-6.82 (m, 1H), 3.85 (s, 3H), 2.93 (q, 2H), 2.36-2.21 (m, 2H), 1.71-1.66 (m, 1H), 1.57-1.52 (m, 1H), 1.24-1.21 (m, 3H).

### Example 77: trans-4-Ethyl-5-(2-(4-fluoro-3-methoxyphenyl)cyclopropyl)-2,2′-bipyrimidine (single enantiomer II)

m/z: 351 [M+H]⁺ observed. ¹H NMR (400 MHz, DMSO-d₆): δ 8.99 (d, 2H), 8.65 (s, 1H), 7.63-7.61 (m, 1H), 7.16-7.11 (m, 1H), 7.07-7.05 (m, 1H), 6.85-6.82 (m, 1H), 3.85 (s, 3H), 2.93 (q, 2H), 2.36-2.21 (m, 2H), 1.71-1.66 (m, 1H), 1.57-1.52 (m, 1H), 1.24-1.21 (m, 3H).

### Example 78: trans-4-Cyclohexyl-5-(2-(4-fluoro-3-methoxyphenyl)cyclopropyl)-2,2′-bipyrimidine (single enantiomer I)

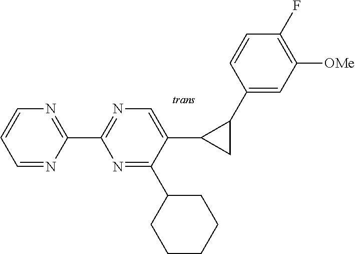 [SMILES: COc1cc(C2CC2c2cnc(-c3ncccn3)nc2C2CCCCC2)ccc1F]

### Example 79: trans-4-Cyclohexyl-5-(2-(4-fluoro-3-methoxyphenyl)cyclopropyl)-2,2′-bipyrimidine (single enantiomer II)

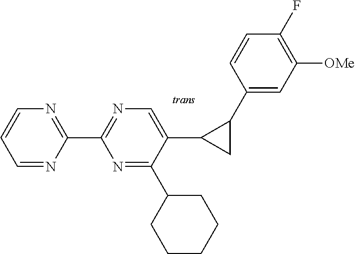 [SMILES: COc1cc(C2CC2c2cnc(-c3ncccn3)nc2C2CCCCC2)ccc1F]

A mixture of enantiomers (55 mg) was separated by HPLC on a CHIRALPAK® AD-H column using n-hexane and EtOH (25:75) to give trans-4-cyclohexyl-5-(2-(4-fluoro-3-methoxyphenyl)cyclopropyl)-2,2′-bipyrimidine (single enantiomer I) as a brown solid (faster eluting enantiomer, 19 mg, 35%, m/z: 405 [M+H]⁺ observed), and trans-4-cyclohexyl-5-(2-(4-fluoro-3-methoxyphenyl)cyclopropyl)-2,2′-bipyrimidine (single enantiomer II) as a brown solid (slower eluting enantiomer, 19 mg, 35%, m/z: 405 [M+H]⁺ observed).

### Example 78: trans-4-Cyclohexyl-5-(2-(4-fluoro-3-methoxyphenyl)cyclopropyl)-2,2′-bipyrimidine (single enantiomer I)

m/z: 405 [M+H]⁺ observed. ¹H NMR (400 MHz, DMSO-d₆): δ 8.98 (d, 2H), 8.66 (s, 1H), 7.63-7.61 (m, 1H), 7.17-7.12 (m, 1H), 7.08-7.05 (m, 1H), 6.84-6.80 (m, 1H), 3.05 (s, 3H), 3.20-3.09 (m, 1H), 2.37-2.32 (m, 1H), 2.26-2.21 (m, 1H), 1.81-1.52 (m, 9H), 1.23-1.10 (m, 3H).

### Example 79: trans-4-Cyclohexyl-5-(2-(4-fluoro-3-methoxyphenyl)cyclopropyl)-2,2′-bipyrimidine (single enantiomer II)

m/z: 405 [M+H]⁺ observed. ¹H NMR (400 MHz, DMSO-d₆): δ 8.98 (d, 2H), 8.66 (s, 1H), 7.63-7.61 (m, 1H), 7.17-7.12 (m, 1H), 7.08-7.05 (m, 1H), 6.84-6.80 (m, 1H), 3.05 (s, 3H), 3.20-3.09 (m, 1H), 2.37-2.32 (m, 1H), 2.26-2.21 (m, 1H), 1.81-1.52 (m, 9H), 1.23-1.10 (m, 3H).

### Example 80: trans-5-(2-(4-Fluoro-3-methoxyphenyl)cyclopropyl)-4-methoxy-2,2′-bipyrimidine (single enantiomer I)

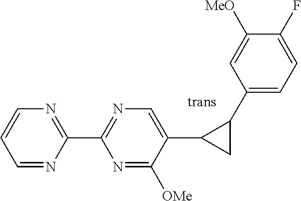 [SMILES: COc1cc(C2CC2c2cnc(-c3ncccn3)nc2OC)ccc1F]

### Example 81: trans-5-(2-(4-Fluoro-3-methoxyphenyl)cyclopropyl)-4-methoxy-2,2′-bipyrimidine (single enantiomer II)

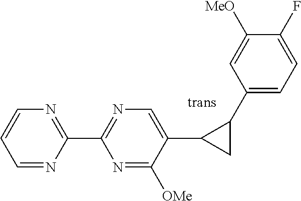 [SMILES: COc1cc(C2CC2c2cnc(-c3ncccn3)nc2OC)ccc1F]

### (E)-2-(4-Fluoro-3-methoxystyryl)-4,4,5,5-tetramethyl-1,3,2-dioxaborolane

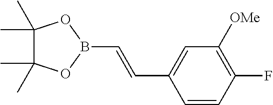 [SMILES: COc1cc(/C=C/B2OC(C)(C)C(C)(C)O2)ccc1F]

To a solution of 4-bromo-1-fluoro-2-methoxybenzene (4.0 g, 19.5 mmol) in toluene (20 mL) at rt was added triethylamine (8.2 mL, 59 mmol) and 4,4,5,5-tetramethyl-2-vinyl-1,3,2-dioxaborolane (6.0 g, 39 mmol). The mixture was purged with N₂ gas for 10 min. Bis(tri-tert-butylphosphine)palladium (100 mg, 0.19 mmol) was added and the mixture was purged with N₂ gas for 10 min. The reaction mixture was heated at 120° C. for 16 h in a sealed tube. The reaction mixture was cooled to rt, poured into ice water (200 mL), and extracted with EtOAc (2×300 mL). The combined organic phase was washed with saturated aqueous brine solution (100 mL), dried over anhydrous sodium sulfate, filtered, and evaporated under reduced pressure. The residue was purified by normal phase SiO₂ chromatography (0-10% EtOAc/petroleum ether) to give (E)-2-(4-fluoro-3-methoxystyryl)-4,4,5,5-tetramethyl-1,3,2-dioxaborolane as an orange solid (2.6 g, 48% yield, m/z: 279 [M+H]⁺ observed). ¹H NMR (400 MHz, CDCl₃): δ 7.15 (d, 1H), 7.12-7.09 (m, 1H), 7.05-6.99 (m, 2H), 6.06 (d, 1H), 3.88 (s, 3H), 1.31 (s, 12H).

### (E)-2-Chloro-5-(4-fluoro-3-methoxystyryl)-4-methoxypyrimidine

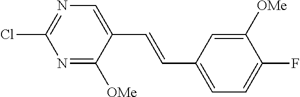 [SMILES: COc1cc(/C=C/c2cnc(Cl)nc2OC)ccc1F]

To a solution of (E)-2-(4-fluoro-3-methoxystyryl)-4,4,5,5-tetramethyl-1,3,2-dioxaborolane (2.2 g, 7.9 mmol) in 1,4-dioxane/water (1:1, 20 mL) was added 5-bromo-2-chloro-4-methoxypyrimidine (2.64 g, 11.8 mmol) and Na2CO3 (1.6 g, 15 mmol). Tetrakis(triphenylphosphine)palladium(0) (914 mg, 0.79 mmol) was added and the mixture purged with N₂ gas for 10 min. The reaction mixture was heated at 90° C. for 16 h in a sealed tube. The reaction mixture was cooled to rt, diluted with water (200 mL), and extracted with EtOAc (2×200 mL). The combined organic phase was washed with saturated aqueous brine solution (200 mL), dried over anhydrous sodium sulfate, filtered, and evaporated under reduced pressure. The residue was purified by normal phase SiO₂ chromatography (0-10% EtOAc/petroleum ether) to give (E)-2-chloro-5-(4-fluoro-3-methoxystyryl)-4-methoxypyrimidine as yellow solid (1.1 g, 47% yield, m/z: 295 [M+H]⁺ observed).

### trans-2-Chloro-5-(2-(4-fluoro-3-methoxyphenyl)cyclopropyl)-4-methoxypyrimidine

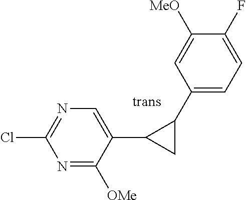 [SMILES: COc1cc(C2CC2c2cnc(Cl)nc2OC)ccc1F]

To a solution of (E)-2-chloro-5-(4-fluoro-3-methoxystyryl)-4-methoxypyrimidine (1.0 g, 3.3 mmol) in THF (5 mL) at 0° C. was added Pd₃(OAc)₆ (228 mg, 0.34 mmol), followed by freshly prepared ethereal diazomethane [prepared from N-methyl-N-nitroso urea (7.0 g, 68.0 mmol), KOH solution (50% aqueous solution, 50 mL) and Et₂O (50 mL) at 0° C.] and the mixture was stirred at 0-5° C. for 16 h. The mixture was filtered through a CELITE° pad and the filtrate was concentrated under reduced pressure. The residue was dissolved in THF (5 mL) at 0° C. and Pd₃(OAc)₆ (228 mg, 0.34 mmol) was added, followed by freshly prepared ethereal diazomethane [prepared from N-methyl-N-nitroso urea (7.0 g, 68.0 mmol), KOH solution (50% aqueous solution, 50 mL) and Et₂O (50 mL) at 0° C.]. The reaction mixture was stirred at 0-5° C. for 16 h. The mixture was filtered through a CELITE° pad and the filtrate was concentrated under reduced pressure. The residue was purified by normal phase SiO₂ chromatography (0-10% EtOAc/petroleum ether) to give trans-2-chloro-5-(2-(4-fluoro-3-methoxyphenyl)cyclopropyl)-4-methoxypyrimidine as a yellow oil (0.40 g, 38% yield, m/z: 309 [M+H]⁺ observed).

### trans-5-(2-(4-Fluoro-3-methoxyphenyl)cyclopropyl)-4-methoxy-2,2′-bipyrimidine:

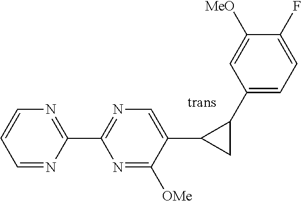 [SMILES: COc1cc(C2CC2c2cnc(-c3ncccn3)nc2OC)ccc1F]

To a solution of trans-2-chloro-5-(2-(4-fluoro-3-methoxyphenyl)cyclopropyl)-4-methoxypyrimidine (0.40 g, 1.3 mmol) in DIVIF (5 mL) at rt was added 2-(tributylstannyl)pyrimidine (0.41 mL, 1.3 mmol), followed by tetraethylammonium chloride (0.21 g, 1.3 mmol) and K₂CO₃ (0.36 g, 2.6 mmol). The mixture was purged with N₂ gas for 10 min. Bis(triphenylphosphine)palladium(II) dichloride (90 mg, 0.13 mmol) was added and the mixture was purged with N₂ gas for 10 min. The reaction mixture was stirred at 110° C. for 16 h. The mixture was cooled to rt, diluted with water (100 mL), and extracted with EtOAc (2×200 mL). The combined organic layer was washed with saturated aqueous brine solution (100 mL), dried over anhydrous sodium sulfate, filtered, and evaporated under reduced pressure. The residue was purified by reverse phase HPLC to give trans-5-(2-(4-fluoro-3-methoxyphenyl)cyclopropyl)-4-methoxy -2,2′-bipyrimidine (90 mg, 19% yield, m/z: 353 [M+H]⁺ observed).

A mixture of enantiomers (90 mg) was separated by SFC (supercritical fluid chromatography) on a CHIRALPAK® IG column using liquid CO₂ and EtOH [60:40; 0.1% methanolic NH₃ as modifier)] to give trans-5-(2-(4-fluoro-3-methoxyphenyl)cyclopropyl)-4-methoxy-2,2′-bipyrimidine (single enantiomer I) as an off-white solid (faster eluting enantiomer, 12 mg, 12%, m/z: 353 [M+H]⁺ observed), and trans-5-(2-(4-fluoro-3-methoxyphenyl)cyclopropyl)-4-methoxy-2,2′-bipyrimidine (single enantiomer II) as an off-white solid (slower eluting enantiomer, 11 mg, 12%, m/z: 353 [M+H]⁺ observed).

### Example 80: trans-5-(2-(4-Fluoro-3-methoxyphenyl)cyclopropyl)-4-methoxy-2,2′-bipyrimidine (single enantiomer I)

m/z: 353 [M+H]⁺ observed. ¹H NMR (400 MHz, DMSO-d₆): δ 8.98 (d, 2H), 8.48 (s, 1H), 7.62 (t, 1H), 7.14-7.09 (m, 1H), 7.04-7.02 (m, 1H), 6.80-6.77 (m,1H), 4.03 (s, 3H), 3.85 (s, 3H), 2.43-2.39 (m, 1H), 2.25-2.20 (m, 1H), 1.70-1.65 (m, 1H), 1.56-1.50 (m, 1H).

### Example 81: trans-5-(2-(4-Fluoro-3-methoxyphenyl)cyclopropyl)-4-methoxy-2,2′-bipyrimidine (single enantiomer II)

m/z: 353 [M+H]⁺ observed. ¹H NMR (400 MHz, DMSO-d₆): δ 8.98 (d, 2H), 8.48 (s, 1H), 7.62 (t, 1H), 7.14-7.09 (m, 1H), 7.04-7.02 (m, 1H), 6.80-6.77 (m,1H), 4.03 (s, 3H), 3.85 (s, 3H), 2.43-2.39 (m, 1H), 2.25-2.20 (m, 1H), 1.70-1.65 (m, 1H), 1.56-1.50 (m, 1H).

The following examples were prepared in a similar manner as trans-5-(2-(4-fluoro-3-methoxyphenyl)cyclopropyl)-4-methoxy-2,2′-bipyrimidine from an appropriately substituted styryl-4,4,5,5-tetramethyl-1,3,2-dioxaborolane and an appropriately substituted 2-chloropyrimidine:

### Example 82: trans-5-(2-(3-(Azetidin-1-yl)-4-fluorophenyl)cyclopropyl)-2,2′-bipyrimidine (single enantiomer I)

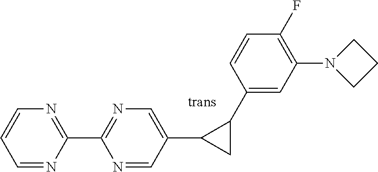 [SMILES: Fc1ccc(C2CC2c2cnc(-c3ncccn3)nc2)cc1N1CCC1]

### Example 83: trans-5-(2-(3-(Azetidin-1-yl)-4-fluorophenyl)cyclopropyl)-2,2′-bipyrimidine (single enantiomer II)

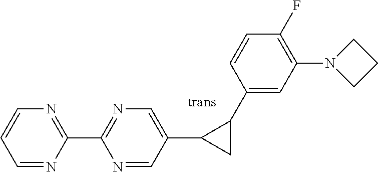 [SMILES: Fc1ccc(C2CC2c2cnc(-c3ncccn3)nc2)cc1N1CCC1]

A mixture of enantiomers (210 mg) was separated by SFC (supercritical fluid chromatography) on a CHIRALPAK® OD-H column using liquid CO₂ and MeOH (65:35) to give trans-5-(2-(3-(azetidin-1-yl)-4-fluorophenyl)cyclopropyl)-2,2′-bipyrimidine (single enantiomer I) as an off-white solid (faster eluting enantiomer, 73 mg, 35%, m/z: 348 [M+H]⁺ observed), and trans-5-(2-(3-(azetidin-1-yl)-4-fluorophenyl)cyclopropyl)-2,2′-bipyrimidine (single enantiomer II) as an off-white solid (slower eluting enantiomer, 62 mg, 30%, m/z: 348 [M+H]⁺ observed).

### Example 82: trans-5-(2-(3-(Azetidin-1-yl)-4-fluorophenyl)cyclopropyl)-2,2′-bipyrimidine (single enantiomer I)

m/z: 348 [M+H]+ observed. ¹H NMR (400 MHz, DMSO-d₆): δ 8.99-8.98 (m, 2H), 8.83 (s, 2H), 7.63-7.61 (m, 1H), 6.97-6.92 (m, 1H), 6.55-6.51 (m, 1H), 6.38-6.35 (m, 1H), 3.91-3.87 (m, 4H), 2.47-2.42 (m, 1H), 2.32-2.23 (m, 3H), 1.69-1.64 (m, 1H), 1.60-1.55 (m, 1H).

### Example 83: trans-5-(2-(3-(Azetidin-1-yl)-4-fluorophenyl)cyclopropyl)-2,2′-bipyrimidine (single enantiomer II)

m/z: 348 [M+H]⁺ observed. ¹H NMR (400 MHz, DMSO-d₆): δ 8.99-8.98 (m, 2H), 8.83 (s, 2H), 7.63-7.61 (m, 1H), 6.97-6.92 (m, 1H), 6.55-6.51 (m, 1H), 6.38-6.35 (m, 1H), 3.91-3.87 (m, 4H), 2.47-2.42 (m, 1H), 2.32-2.23 (m, 3H), 1.69-1.64 (m, 1H), 1.60-1.55 (m, 1H).

### Example 84: trans-5-(2-(3,4-Difluoro-5-methoxyphenyl)cyclopropyl)-2,2′-bipyrimidine (single enantiomer I)

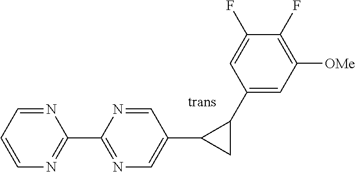 [SMILES: COc1cc(C2CC2c2cnc(-c3ncccn3)nc2)cc(F)c1F]

### Example 85: trans-5-(2-(3,4-Difluoro-5-methoxyphenyl)cyclopropyl)-2,2′-bipyrimidine (single enantiomer II)

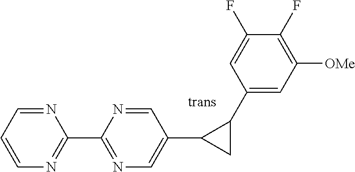 [SMILES: COc1cc(C2CC2c2cnc(-c3ncccn3)nc2)cc(F)c1F]

A mixture of enantiomers (110 mg) was separated by SFC (supercritical fluid chromatography) on a CHIRALPAK® OD-H column using liquid CO₂ and MeOH (50:50) to give trans-5-(2-(3,4-difluoro-5-methoxyphenyl)cyclopropyl)-2,2′-bipyrimidine (single enantiomer I) as an off-white solid (faster eluting enantiomer, 35 mg, 32%, m/z: 341 [M+H]⁺ observed), and trans-5-(2-(3,4-difluoro-5-methoxyphenyl)cyclopropyl)-2,2′-bipyrimidine (single enantiomer II) as an off-white solid (slower eluting enantiomer, 27 mg, 25%, m/z: 341 [M+H]⁺ observed).

### Example 84: trans-5-(2-(3,4-Difluoro-5-methoxyphenyl)cyclopropyl)-2,2′-bipyrimidine (single enantiomer I)

m/z: 341 [M+H]⁺ observed. ¹H NMR (400 MHz, DMSO-d₆): δ 8.99 (d, 2H), 8.84 (s, 2H), 7.62 (t, 1H), 6.94-6.85 (m, 2H), 3.88 (s, 3H), 2.55-2.50 (m, 1H), 2.42-2.37 (m, 1H), 1.77-1.67 (m, 2H).

### Example 85: trans-5-(2-(3,4-Difluoro-5-methoxyphenyl)cyclopropyl)-2,2′-bipyrimidine (single enantiomer II)

m/z: 341 [M+H]⁺ observed. ¹H NMR (400 MHz, DMSO-d₆): δ 8.99 (d, 2H), 8.84 (s, 2H), 7.62 (t, 1H), 6.94-6.85 (m, 2H), 3.88 (s, 3H), 2.55-2.50 (m, 1H), 2.42-2.37 (m, 1H), 1.77-1.67 (m, 2H).

### Example 86: trans-5-(2-(4-Chloro-2-fluoro-5-methoxyphenyl)cyclopropyl)-2,2′-bipyrimidine (single enantiomer I)

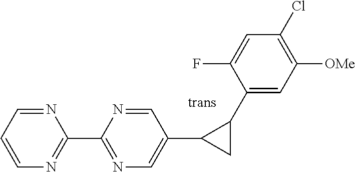 [SMILES: COc1cc(C2CC2c2cnc(-c3ncccn3)nc2)c(F)cc1Cl]

### Example 87: trans-5-(2-(4-Chloro-2-fluoro-5-methoxyphenyl)cyclopropyl)-2,2′-bipyrimidine (single enantiomer II)

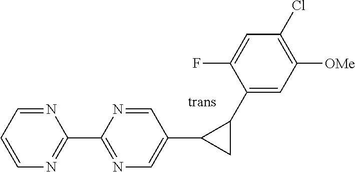 [SMILES: COc1cc(C2CC2c2cnc(-c3ncccn3)nc2)c(F)cc1Cl]

A mixture of enantiomers of trans-5-(2-(4-chloro-2-fluoro-5-methoxyphenyl)cyclopropyl)-2,2′-bipyrimidine (100 mg) was separated by chiral chromatography on a LUX® Amylose-2 column using n-Hexane:Ethanol (30:70) to give trans-5-(2-(4-chloro-2-fluoro-5-methoxyphenyl)cyclopropyl)-2,2′-bipyrimidine (single enantiomer I) as a brick red solid (faster eluting enantiomer, 28 mg, 28%, m/z: 357 [M+H]⁺ observed), and trans-5-(2-(4-chloro-2-fluoro-5-methoxyphenyl)cyclopropyl)-2,2′-bipyrimidine (single enantiomer II) as a pale orange solid (slower eluting enantiomer, 28 mg, 28%, m/z: 357 [M+H]⁺ observed).

### Example 86: trans-5-(2-(4-Chloro-2-fluoro-5-methoxyphenyl)cyclopropyl)-2,2′-bipyrimidine (single enantiomer I)

m/z: 357 [M+H]⁺ observed. ¹H NMR (400 MHz, DMSO-d₆): δ 8.99 (d, 2H), 8.89 (s, 2H), 7.63 (t, 1H), 7.41-7.39 (d, 1H), 6.93-6.91 (d, 1H), 3.88 (s, 3H), 2.55-2.49 (m, 2H), 1.81-1.76 (m, 2H).

### Example 87: trans-5-(2-(4-Chloro-2-fluoro-5-methoxyphenyl)cyclopropyl)-2,2′-bipyrimidine (single enantiomer II)

m/z: 357 [M+H]⁺ observed. ¹H NMR (400 MHz, DMSO-d₆): δ 8.99 (d, 2H), 8.89 (s, 2H), 7.63 (t, 1H), 7.41-7.39 (d, 1H), 6.93-6.91 (d, 1H), 3.88 (s, 3H), 2.55-2.49 (m, 2H), 1.81-1.76 (m, 2H).

### Example 88: trans-5-(2-(2,4-Dichloro-5-methoxyphenyl)cyclopropyl)-2,2′-bipyrimidine (single enantiomer I)

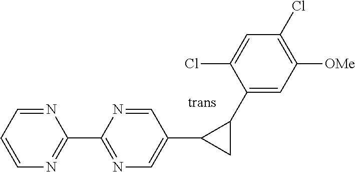 [SMILES: COc1cc(C2CC2c2cnc(-c3ncccn3)nc2)c(Cl)cc1Cl]

### Example 89: trans-5-(2-(2,4-Dichloro-5-methoxyphenyl)cyclopropyl)-2,2′-bipyrimidine (single enantiomer II)

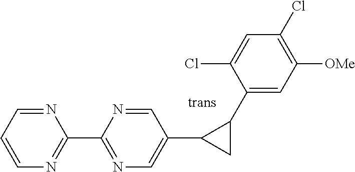 [SMILES: COc1cc(C2CC2c2cnc(-c3ncccn3)nc2)c(Cl)cc1Cl]

A mixture of enantiomers of trans-5-(2-(2,4-dichloro-5-methoxyphenyl)cyclopropyl)-2,2′-bipyrimidine (105 mg) was separated by SFC (supercritical fluid chromatography) on a LUX® Amylose-2 column using liquid CO₂ and MeOH (50:50) to give trans-5-(2-(2,4-dichloro-5-methoxyphenyl)cyclopropyl)-2,2′-bipyrimidine (single enantiomer I) as a brick red solid (faster eluting enantiomer, 10 mg, 9%, m/z: 373 [M+H]⁺ observed), and trans-5-(2-(2,4-dichloro-5-methoxyphenyl)cyclopropyl)-2,2′-bipyrimidine (single enantiomer II) as a pale orange solid (slower eluting enantiomer, 16 mg, 15%, m/z: 373 [M+H]⁺ observed).

### Example 88: trans-5-(2-(2,4-Dichloro-5-methoxyphenyl)cyclopropyl)-2,2′-bipyrimidine (single enantiomer I)

m/z: 373 [M+H]⁺ observed. ¹H NMR (400 MHz, DMSO-d₆): δ 8.99 (d, 2H), 8.91 (s, 2H), 7.63 (t, 1H), 7.58 (s, 1H), 6.97 (s, 1H), 3.92 (s, 3H), 2.67-2.60 (m, 1H), 2.38-2.32 (m, 1H), 1.89-1.78 (m, 2H).

### Example 89: trans-5-(2-(2,4-Dichloro-5-methoxyphenyl)cyclopropyl)-2,2′-bipyrimidine (single enantiomer II)

m/z: 373 [M+H]⁺ observed. ¹H NMR (400 MHz, DMSO-d₆): δ 8.99 (d, 2H), 8.91 (s, 2H), 7.63 (t, 1H), 7.58 (s, 1H), 6.97 (s, 1H), 3.92 (s, 3H), 2.67-2.60 (m, 1H), 2.38-2.32 (m, 1H), 1.89-1.78 (m, 2H).

### Example 90: trans-5-(2-(4-Chloro-3-fluoro-5-(pyrrolidin-1-yl)phenyl)cyclopropyl)-2,2′-bipyrimidine (single enantiomer I)

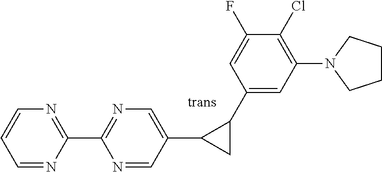 [SMILES: Fc1cc(C2CC2c2cnc(-c3ncccn3)nc2)cc(N2CCCC2)c1Cl]

### Example 91: trans-5-(2-(4-Chloro-3-fluoro-5-(pyrrolidin-1-yl)phenyl)cyclopropyl)-2,2′-bipyrimidine (single enantiomer II)

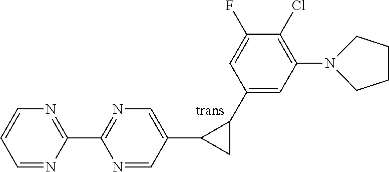 [SMILES: Fc1cc(C2CC2c2cnc(-c3ncccn3)nc2)cc(N2CCCC2)c1Cl]

A mixture of enantiomers of trans-5-(2-(4-chloro-3-fluoro-5-(pyrrolidin-1-yl)phenyl)cyclopropyl)-2,2′-bipyrimidine (170 mg) was separated by SFC (supercritical fluid chromatography) on a CHIRALPAK® OJ-H column using liquid CO₂ and MeOH (50:50) to give trans-5-(2-(4-chloro-3-fluoro-5-(pyrrolidin-1-yl)phenyl)cyclopropyl)-2,2′-bipyrimidine (single enantiomer I) as an off-white solid (faster eluting enantiomer, 48 mg, 28%, m/z: 396 [M+H]⁺ ob served), and trans-5-(2-(4-chloro-3-fluoro-5-(pyrrolidin-1-yl)phenyl)cyclopropyl)-2,2′-bipyrimidine (single enantiomer II) as an off-white solid (slower eluting enantiomer, 47 mg, 27%, m/z: 396 [M+H]⁺ observed).

### Example 90: trans-5-(2-(4-Chloro-3-fluoro-5-(pyrrolidin-1-yl)phenyl)cyclopropyl)-2,2′-bipyrimidine (single enantiomer I)

m/z: 396 [M+H]⁺ observed. ¹H NMR (400 MHz, DMSO-d₆): δ 8.99-8.98 (d, 2H), 8.83 (s, 2H), 7.63-7.61 (m, 1H), 6.65-6.62 (m, 2H), 3.40-3.36 (m, 4H), 2.50-2.49 (m, 1H), 2.40-2.38 (m, 1H), 1.89-1.86 (m, 4H), 1.76-1.63 (m, 2H).

### Example 91: trans-5-(2-(4-Chloro-3-fluoro-5-(pyrrolidin-1-yl)phenyl)cyclopropyl)-2,2′-bipyrimidine (single enantiomer II)

m/z: 396 [M+H]⁺ observed. ¹H NMR (400 MHz, DMSO-d₆): δ 8.99-8.98 (d, 2H), 8.83 (s, 2H), 7.63-7.61 (m, 1H), 6.65-6.62 (m, 2H), 3.40-3.36 (m, 4H), 2.50-2.49 (m, 1H), 2.40-2.38 (m, 1H), 1.89-1.86 (m, 4H), 1.76-1.63 (m, 2H).

### Example 92: trans-5-(2-(3,4-Difluoro-5-(pyrrolidin-1-yl)phenyl)cyclopropyl)-2,2′-bipyrimidine (single enantiomer I)

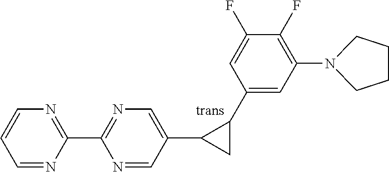 [SMILES: Fc1cc(C2CC2c2cnc(-c3ncccn3)nc2)cc(N2CCCC2)c1F]

### Example 93: trans-5-(2-(3,4-Difluoro-5-(pyrrolidin-1-yl)phenyl)cyclopropyl)-2,2′-bipyrimidine (single enantiomer II)

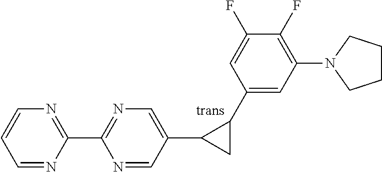 [SMILES: Fc1cc(C2CC2c2cnc(-c3ncccn3)nc2)cc(N2CCCC2)c1F]

A mixture of enantiomers of trans-5-(2-(3,4-difluoro-5-(pyrrolidin-1-yl)phenyl)cyclopropyl)-2,2′-bipyrimidine (140 mg) was separated by SFC (supercritical fluid chromatography) on a CHIRALCEL® OD-H column using liquid CO₂ and MeOH (50:50) to give trans-5-(2-(3,4-difluoro-5-(pyrrolidin-1-yl)phenyl)cyclopropyl)-2,2′-bipyrimidine (single enantiomer I) as an off-white solid (faster eluting enantiomer, 26 mg, 18%, m/z: 380 [M+H]⁺ observed), and trans-5-(2-(3,4-difluoro-5-(pyrrolidin-1-yl)phenyl)cyclopropyl)-2,2′-bipyrimidine (single enantiomer II) as an off-white solid (slower eluting enantiomer, 25 mg, 17%, m/z: 380 [M+H]⁺ observed).

### Example 92: trans-5-(2-(3,4-Difluoro-5-(pyrrolidin-1-yl)phenyl)cyclopropyl)-2,2′-bipyrimidine (single enantiomer I)

m/z: 380 [M+H]⁺ observed. ¹H NMR (400 MHz, DMSO-d₆): δ 8.99 (d, 2H), 8.84 (s, 2H), 7.63-7.61 (m, 1H), 6.50-6.48 (m, 1H), 6.47-6.40 (m, 1H), 3.36-3.26 (m, 4H), 2.50-2.42 (m, 1H), 2.36-2.31 (m, 1H), 1.91-1.97 (m, 4H), 1.71-1.66 (m, 1H), 1.63-1.58 (m, 1H).

### Example 93: trans-5-(2-(3,4-Difluoro-5-(pyrrolidin-1-yl)phenyl)cyclopropyl)-2,2′-bipyrimidine (single enantiomer II)

m/z: 380 [M+H]⁺ observed. ¹H NMR (400 MHz, DMSO-d₆): δ 8.99 (d, 2H), 8.84 (s, 2H), 7.63-7.61 (m, 1H), 6.50-6.48 (m, 1H), 6.47-6.40 (m, 1H), 3.36-3.26 (m, 4H), 2.50-2.42 (m, 1H), 2.36-2.31 (m, 1H), 1.91-1.97 (m, 4H), 1.71-1.66 (m, 1H), 1.63-1.58 (m, 1H).

### Example 94: trans-5-(2-(4-Fluoro-3-((R)-3-methoxypyrrolidin-1-yl)phenyl)cyclopropyl)-2,2′-bipyrimidine (single diastereomer I)

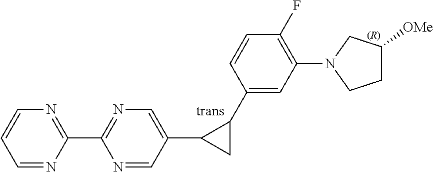

### Example 95: trans-5-(2-(4-Fluoro-3-((R)-3-methoxypyrrolidin-1-yl)phenyl)cyclopropyl)-2,2′-bipyrimidine (single diastereomer II)

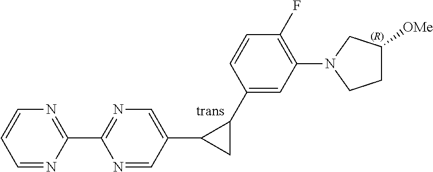

A mixture of diastereomers of trans-5-(2-(4-fluoro-3-((R)-3-methoxypyrrolidin-1-yl)phenyl)cyclopropyl)-2,2′-bipyrimidine (260 mg) was separated by SFC (supercritical fluid chromatography) on a CHIRALCEL® OD-H column using liquid CO₂ and MeOH (50:50) to give trans-5-(2-(4-fluoro-3-((R)-3-methoxypyrrolidin-1-yl)phenyl)cyclopropyl)-2,2′-bipyrimidine (single diastereomer I) as a pale yellow solid (faster eluting diastereomer, 55 mg, 21%, m/z: 392 [M+H]⁺ observed), and trans-5-(2-(4-fluoro-3-((R)-3-methoxypyrrolidin-1-yl)phenyl)cyclopropyl)-2,2′-bipyrimidine (single diastereomer II) as an off-white solid (slower eluting diastereomer, 70 mg, 27%, m/z: 392 [M+H]⁺ observed).

### Example 94: trans-5-(2-(4-Fluoro-3-((R)-3-methoxypyrrolidin-1-yl)phenyl)cyclopropyl)-2,2′-bipyrimidine (single diastereomer I)

m/z: 392 [M+H]⁺ observed. ¹H NMR (400 MHz, DMSO-d₆): δ 8.98 (d, 2H), 8.83 (s, 2H), 7.62 (t, 1H), 7.00-6.94 (m, 1H), 6.58-6.56 (m, 1H), 6.52-6.50 (m, 1H), 4.03-4.02 (m, 1H), 3.54-3.52 (m, 1H), 3.40-3.27 (m, 3H), 3.25 (s, 3H), 2.49-2.44 (m, 1H), 2.32-2.30 (m, 1H), 2.00-1.97 (m, 2H), 1.68-1.66 (m, 1H), 1.62-1.58 (m, 1H).

### Example 95: trans-5-(2-(4-Fluoro-3-((R)-3-methoxypyrrolidin-1-yl)phenyl)cyclopropyl)-2,2′-bipyrimidine (single diastereomer II)

m/z: 392 [M+H]⁺ observed. ¹H NMR (400 MHz, DMSO-d₆): δ 8.98 (d, 2H), 8.83 (s, 2H), 7.62 (t, 1H), 7.00-6.94 (m, 1H), 6.58-6.56 (m, 1H), 6.52-6.50 (m, 1H), 4.03-4.02 (m, 1H), 3.54-3.52 (m, 1H), 3.40-3.27 (m, 3H), 3.25 (s, 3H), 2.49-2.44 (m, 1H), 2.32-2.30 (m, 1H), 2.00-1.97 (m, 2H), 1.68-1.66 (m, 1H), 1.62-1.58 (m, 1H).

### Example 96: trans-5-(2-(3,4-Difluoro-5-((R)-3-methoxypyrrolidin-1-yl)phenyl)cyclopropyl)-2,2′-bipyrimidine (single diastereomer I)

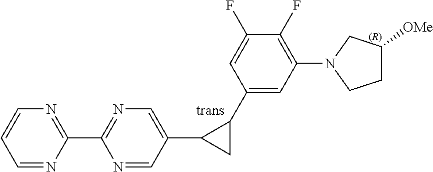

### Example 97: trans-5-(2-(3,4-Difluoro-5-((R)-3-methoxypyrrolidin-1-yl)phenyl)cyclopropyl)-2,2′-bipyrimidine (single diastereomer II)

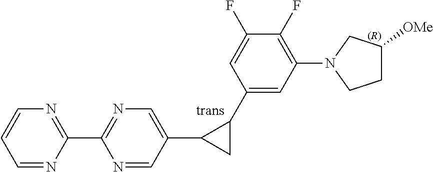

A mixture of diastereomers of trans-5-(2-(3,4-difluoro-5-((R)-3-methoxypyrrolidin-1-yl)phenyl)cyclopropyl)-2,2′-bipyrimidine (245 mg) was separated by SFC (supercritical fluid chromatography) on a CHIRALCEL® OD-H column using liquid CO₂ and MeOH (50:50) to give trans-5-(2-(3,4-difluoro-5-((R)-3-methoxypyrrolidin-1-yl)phenyl)cyclopropyl)-2,2′-bipyrimidine (single diastereomer I) as an off-white solid (faster eluting diastereomer, 62 mg, 25%, m/z: 410 [M+H]⁺ observed), and trans-5-(2-(3,4-difluoro-5-((R)-3-methoxypyrrolidin-1-yl)phenyl)cyclopropyl)-2,2′-bipyrimidine (single diastereomer II) as an off-white solid (slower eluting diastereomer, 58 mg, 23%, m/z: 410 [M+H]⁺ observed).

### Example 96: trans-5-(2-(3,4-Difluoro-5-((R)-3-methoxypyrrolidin-1-yl)phenyl)cyclopropyl)-2,2′-bipyrimidine (single diastereomer I)

m/z: 410 [M+H]⁺ observed. ¹H NMR (400 MHz, DMSO-d₆): δ 8.99 (d, 2H), 8.83 (s, 2H), 7.62 (t, 1H), 6.53-6.48 (m, 1H), 6.42-6.40 (m, 1H), 4.03 (m, 1H), 3.57-3.55 (m, 1H), 3.41-3.34 (m, 3H), 3.25 (s, 3H), 2.49-2.42 (m, 1H), 2.35-2.31 (m, 1H), 2.02-2.00 (m, 2H), 1.70-1.61 (m, 2H).

### Example 97: trans-5-(2-(3,4-Difluoro-5-((R)-3-methoxypyrrolidin-1-yl)phenyl)cyclopropyl)-2,2′-bipyrimidine (single diastereomer II)

m/z: 410 [M+H]⁺ observed. ¹H NMR (400 MHz, DMSO-d₆): δ 8.99 (d, 2H), 8.83 (s, 2H), 7.62 (t, 1H), 6.53-6.48 (m, 1H), 6.42-6.40 (m, 1H), 4.03 (m, 1H), 3.57-3.55 (m, 1H), 3.41-3.34 (m, 3H), 3.25 (s, 3H), 2.49-2.42 (m, 1H), 2.35-2.31 (m, 1H), 2.02-2.00 (m, 2H), 1.70-1.61 (m, 2H).

### Example 98: trans-5-(2-(4-Chloro-3,5-dimethoxyphenyl)cyclopropyl)-2,2′-bipyrimidine (single enantiomer I)

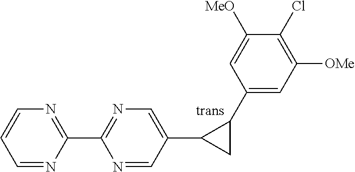 [SMILES: COc1cc(C2CC2c2cnc(-c3ncccn3)nc2)cc(OC)c1Cl]

### Example 99: trans-5-(2-(4-Chloro-3,5-dimethoxyphenyl)cyclopropyl)-2,2′-bipyrimidine (single enantiomer II)

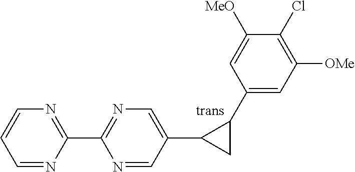 [SMILES: COc1cc(C2CC2c2cnc(-c3ncccn3)nc2)cc(OC)c1Cl]

A mixture of enantiomers of trans-5-(2-(4-chloro-3,5-dimethoxyphenyl)cyclopropyl)-2,2′-bipyrimidine (200 mg) was separated by SFC (supercritical fluid chromatography) on a LUX® Amylose-2 column using liquid CO₂ and MeOH (50:50) to give trans-5-(2-(4-chloro-3,5-dimethoxyphenyl)cyclopropyl)-2,2′-bipyrimidine (single enantiomer I) as an off-white solid (faster eluting enantiomer, 52 mg, 26%, m/z: 369 [M+H]⁺ observed), and trans-5-(2-(4-chloro-3,5-dimethoxyphenyl)cyclopropyl)-2,2′-bipyrimidine (single enantiomer II) as an off-white solid (slower eluting enantiomer, 51 mg, 25%, m/z: 369 [M+H]⁺ observed).

### Example 98: trans-5-(2-(4-Chloro-3,5-dimethoxyphenyl)cyclopropyl)-2,2′-bipyrimidine (single enantiomer I)

m/z: 369 [M+H]⁺ observed. ¹H NMR (400 MHz, DMSO-d₆): δ 8.99 (d, 2H), 8.85 (s, 2H), 7.62 (t, 1H), 6.64 (s, 2H), 3.85 (s, 6H), 2.55-2.51 (m, 1H), 2.49-2.43 (m, 1H), 1.77-1.73 (m, 2H).

### Example 99: trans-5-(2-(4-Chloro-3,5-dimethoxyphenyl)cyclopropyl)-2,2′-bipyrimidine (single enantiomer II)

m/z: 369 [M+H]⁺ observed. ¹H NMR (400 MHz, DMSO-d₆): δ 8.99 (d, 2H), 8.85 (s, 2H), 7.62 (t, 1H), 6.64 (s, 2H), 3.85 (s, 6H), 2.55-2.51 (m, 1H), 2.49-2.43 (m, 1H), 1.77-1.73 (m, 2H).

### Example 100: trans-5-(2-(4-Fluoro-3-methoxy-5-(pyrrolidin-1-yl)phenyl)cyclopropyl)-2,2′-bipyrimidine (single enantiomer I)

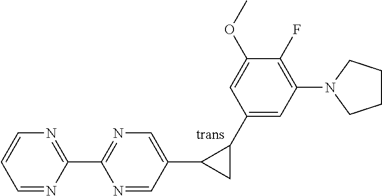 [SMILES: COc1cc(C2CC2c2cnc(-c3ncccn3)nc2)cc(N2CCCC2)c1F]

### Example 101: trans-5-(2-(4-Fluoro-3-methoxy-5-(pyrrolidin-1-yl)phenyl)cyclopropyl)-2,2′-bipyrimidine (single enantiomer II)

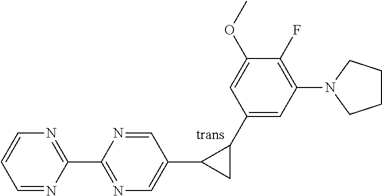 [SMILES: COc1cc(C2CC2c2cnc(-c3ncccn3)nc2)cc(N2CCCC2)c1F]

A mixture of enantiomers of trans-5-(2-(4-fluoro-3-methoxy-5-(pyrrolidin-1-yl)phenyl)cyclopropyl)-2,2′-bipyrimidine (200 mg) was separated by SFC (supercritical fluid chromatography) on a LUX® Amylose-2 column using liquid CO₂ and MeOH (50:50) to give trans-5-(2-(4-fluoro-3-methoxy-5-(pyrrolidin-1-yl)phenyl)cyclopropyl)-2,2′-bipyrimidine (single enantiomer I) as an off-white solid (faster eluting enantiomer, 36 mg, 18%, m/z: 392 [M+H]⁺ observed), and of trans-5-(2-(4-fluoro-3-methoxy-5-(pyrrolidin-1-yl)phenyl)cyclopropyl)-2,2′-bipyrimidine (single enantiomer II) as an off-white solid (slower eluting enantiomer, 34 mg, 17%, m/z: 392 [M+H]⁺ observed).

### Example 100: trans-5-(2-(4-Fluoro-3-methoxy-5-(pyrrolidin-1-yl)phenyl)cyclopropyl)-2,2′-bipyrimidine (single enantiomer I)

m/z: 392 [M+H]⁺ observed. ¹H NMR (400 MHz, DMSO-d₆): δ 8.99 (d, 2H), 8.83 (s, 2H), 7.62 (t, 1H), 6.34-6.31 (m, 1H), 6.22-6.20 (m, 1H), 3.78 (s, 3H), 3.32-3.30 (m, 4H), 2.43-2.40 (m, 1H), 2.36-2.31 (m, 1H),1.88-187(m, 4H), 1.67-1.62 (m, 2H).

### Example 101: trans-5-(2-(4-Fluoro-3-methoxy-5-(pyrrolidin-1-yl)phenyl)cyclopropyl)-2,2′-bipyrimidine (single enantiomer II)

m/z: 392 [M+H]⁺ observed. ¹H NMR (400 MHz, DMSO-d₆): δ 8.99 (d, 2H), 8.83 (s, 2H), 7.62 (t, 1H), 6.34-6.31 (m, 1H), 6.22-6.20 (m, 1H), 3.78 (s, 3H), 3.32-3.30 (m, 4H), 2.43-2.40 (m, 1H), 2.36-2.31 (m, 1H),1.88-187(m, 4H), 1.67-1.62 (m, 2H).

### Example 102: trans-5-(2-(3,4-Difluoro-5-((S)-3-methoxypyrrolidin-1-yl)phenyl)cyclopropyl)-2,2′-bipyrimidine (single diastereomer I)

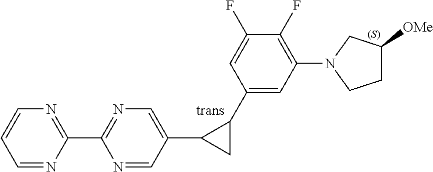

### Example 103: trans-5-(2-(3,4-Difluoro-5-((S)-3-methoxypyrrolidin-1-yl)phenyl)cyclopropyl)-2,2′-bipyrimidine (single diastereomer II)

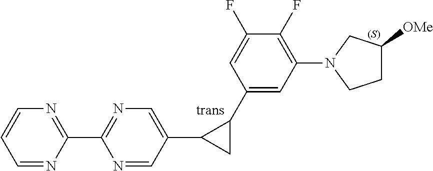

A mixture of diastereomers of trans-5-(2-(3,4-difluoro-5-((S)-3-methoxypyrrolidin-1-yl)phenyl)cyclopropyl)-2,2′-bipyrimidine (110 mg) was separated by SFC (supercritical fluid chromatography) on a CHIRALCEL® OD-H column using liquid CO₂ and MeOH (50:50) to give trans-5-(2-(3,4-difluoro-5-((S)-3-methoxypyrrolidin-1-yl)phenyl)cyclopropyl)-2,2′-bipyrimidine (single diastereomer I) as an off-white solid (faster eluting diastereomer, 30 mg, 27%, m/z: 410 [M+H]⁺ observed), and trans-5-(2-(3,4-difluoro-5-((S)-3-methoxypyrrolidin-l-yl)phenyl)cyclopropyl)-2,2′-bipyrimidine (single diastereomer II) as an off-white solid (slower eluting diastereomer, 27 mg, 24%, m/z: 410 [M+H]⁺ observed).

### Example 102: trans-5-(2-(3,4-Difluoro-5-((S)-3-methoxypyrrolidin-1-yl)phenyl)cyclopropyl)-2,2′-bipyrimidine (single diastereomer I)

m/z: 410 [M+H]⁺ observed. ¹H NMR (400 MHz, DMSO-d₆): δ 8.99 (d, 2H), 8.83 (s, 2H), 7.63 (t, 1H), 6.53-6.48 (m, 1H), 6.42-6.40 (m, 1H), 4.03 (s, 1H), 3.57-3.55 (m, 1H), 3.41-3.34 (m, 3H), 3.25 (s, 3H), 2.49-2.42 (m, 1H), 2.35-2.31 (m, 1H), 2.02-2.00 (m, 2H), 1.70-1.61 (m, 2H).

### Example 103: trans-5-(2-(3,4-Difluoro-5-((S)-3-methoxypyrrolidin-1-yl)phenyl)cyclopropyl)-2,2′-bipyrimidine (single diastereomer II)

m/z: 410 [M+H]⁺ observed. ¹H NMR (400 MHz, DMSO-d₆): δ 8.99 (d, 2H), 8.83 (s, 2H), 7.63 (t, 1H), 6.53-6.48 (m, 1H), 6.42-6.40 (m, 1H), 4.03 (s, 1H), 3.57-3.55 (m, 1H), 3.41-3.34 (m, 3H), 3.25 (s, 3H), 2.49-2.42 (m, 1H), 2.35-2.31 (m, 1H), 2.02-2.00 (m, 2H), 1.70-1.61 (m, 2H).

### Example 104: trans-(3R)-1-(5-(2-([2,2′-Bipyrimidin]-5-yl)cyclopropyl)-2,3-difluorophenyl)pyrrolidin-3-ol (single diastereomer I)

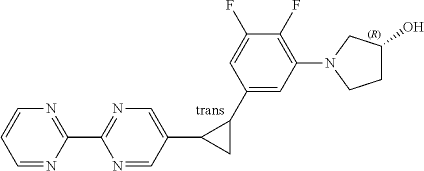

### Example 105: trans-(3R)-1-(5-(2-([2,2′-Bipyrimidin]-5-yl)cyclopropyl)-2,3-difluorophenyl)pyrrolidin-3-ol (single diastereomer II)

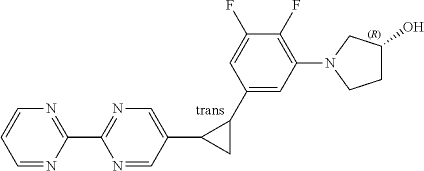

A mixture of diastereomers of trans-(3R)-1-(5-(2-([2,2′-bipyrimidin]-5-yl)cyclopropyl)-2,3-difluorophenyl)pyrrolidin-3-ol (110 mg) was separated by SFC (supercritical fluid chromatography) on a CHIRALCEL® OD-H column using liquid CO₂ and MeOH (50:50) to give trans-(3R)-1-(5-(2-([2,2′-bipyrimidin]-5-yl)cyclopropyl)-2,3-difluorophenyl)pyrrolidin-3-ol (single diastereomer I) as a pale yellow solid (faster eluting diastereomer, 33 mg, 30%, m/z: 396 [M+H]⁺ observed), and trans-(3R)-1-(5-(2-([2,2′-bipyrimidin]-5-yl)cyclopropyl)-2,3-difluorophenyl)pyrrolidin-3-ol (single diastereomer II) as a pale yellow solid (slower eluting diastereomer, 35 mg, 31%, m/z: 396 [M+H]⁺ observed).

### Example 104: trans-(3R)-1-(5-(2-([2,2′-Bipyrimidin]-5-yl)cyclopropyl)-2,3-difluorophenyl)pyrrolidin-3-ol (single diastereomer I)

m/z: 396 [M+H]⁺ observed. ¹H NMR (400 MHz, DMSO-d₆): δ 8.99 (d, 2H), 8.82 (s, 2H), 7.62 (t, 1H), 6.49-6.44 (m, 1H), 6.40-6.38 (m, 1H), 4.96-4.94 (m, 1H), 4.35-4.33 (m, 1H), 3.61-3.46 (m, 2H), 3.39-3.36 (m, 1H), 3.22-3.19 (m, 1H), 2.49-2.43 (m, 1H), 2.35-2.32 (m, 1H), 1.99-1.94 (m, 1H), 1.85-1.84 (m, 1H), 1.70-1.68 (m, 1H), 1.62-1.60 (m, 1H).

### Example 105: trans-(3R)-1-(5-(2-([2,2′-Bipyrimidin]-5-yl)cyclopropyl)-2,3-difluorophenyl)pyrrolidin-3-ol (single diastereomer II)

m/z: 396 [M+H]⁺ observed. ¹H NMR (400 MHz, DMSO-d₆): δ 8.99 (d, 2H), 8.82 (s, 2H), 7.62 (t, 1H), 6.49-6.44 (m, 1H), 6.40-6.38 (m, 1H), 4.96-4.94 (m, 1H), 4.35-4.33 (m, 1H), 3.61-3.46 (m, 2H), 3.39-3.36 (m, 1H), 3.22-3.19 (m, 1H), 2.49-2.43 (m, 1H), 2.35-2.32 (m, 1H), 1.99-1.94 (m, 1H), 1.85-1.84 (m, 1H), 1.70-1.68 (m, 1H), 1.62-1.60 (m, 1H).

### Example 106: trans-(3S)-1-(5-(2-([2,2′-Bipyrimidin]-5-yl)cyclopropyl)-2,3-difluorophenyl)pyrrolidin-3-ol (single diastereomer I)

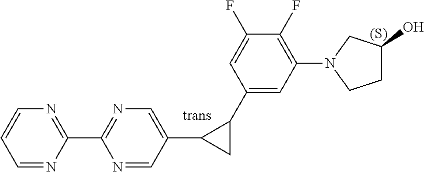

### Example 107: trans-(3S)-1-(5-(2-([2,2′-Bipyrimidin]-5-yl)cyclopropyl)-2,3-difluorophenyl)pyrrolidin-3-ol (single diastereomer II)

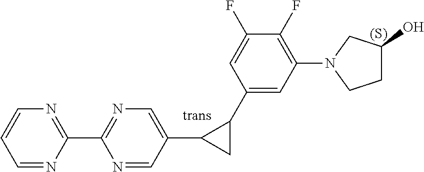

A mixture of diastereomers of trans-(3S)-1-(5-(2-([2,2′-bipyrimidin]-5-yl)cyclopropyl)-2,3-difluorophenyl)pyrrolidin-3-ol (100 mg) was separated by SFC (supercritical fluid chromatography) on a CHIRALCEL® OD-H column using liquid CO₂ and MeOH (55:45) to give trans-(3S)-1-(5-(2-([2,2′-bipyrimidin]-5-yl)cyclopropyl)-2,3-difluorophenyl)pyrrolidin-3-ol (single diastereomer I) as an off-white solid (faster eluting diastereomer, 32 mg, 32%, m/z: 396 [M+H]⁺ observed), and trans-(3S)-1-(5-(2-([2,2′-bipyrimidin]-5-yl)cyclopropyl)-2,3-difluorophenyl)pyrrolidin-3-ol (single diastereomer II) as a pale yellow solid (slower eluting diastereomer, 35 mg, 35%, m/z: 396 [M+H]⁺ observed).

### Example 106: trans-(3S)-1-(5-(2-([2,2′-Bipyrimidin]-5-yl)cyclopropyl)-2,3-difluorophenyl)pyrrolidin-3-ol (single diastereomer I)

m/z: 396 [M+H]⁺ observed. ¹H NMR (400 MHz, DMSO-d₆): δ 8.99 (d, 2H), 8.82 (s, 2H), 7.62 (t, 1H), 6.49-6.44 (m, 1H), 6.40-6.38 (m, 1H), 4.94 (d, 1H), 4.35-4.33 (m, 1H), 3.61-3.46 (m, 2H), 3.39-3.36 (m, 1H), 3.22-3.19 (m, 1H), 2.49-2.43 (m, 1H), 2.35-2.32 (m, 1H), 1.99-1.94 (m, 1H), 1.85-1.84 (m, 1H), 1.70-1.60 (m, 2H).

### Example 107: trans-(3S)-1-(5-(2-([2,2′-Bipyrimidin]-5-yl)cyclopropyl)-2,3-difluorophenyl)pyrrolidin-3-ol (single diastereomer II)

m/z: 396 [M+H]⁺ observed. ¹H NMR (400 MHz, DMSO-d₆): δ 8.99 (d, 2H), 8.82 (s, 2H), 7.62 (t, 1H), 6.49-6.44 (m, 1H), 6.40-6.38 (m, 1H), 4.94 (d, 1H), 4.35-4.33 (m, 1H), 3.61-3.46 (m, 2H), 3.39-3.36 (m, 1H), 3.22-3.19 (m, 1H), 2.49-2.43 (m, 1H), 2.35-2.32 (m, 1H), 1.99-1.94 (m, 1H), 1.85-1.84 (m, 1H), 1.70-1.60 (m, 2H).

### Example 108: trans-5-(2-(3-Chloro-4-fluoro-5-(pyrrolidin-1-yl)phenyl)cyclopropyl)-2,2′-bipyrimidine (single enantiomer I)

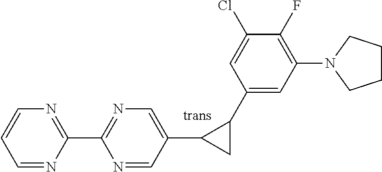 [SMILES: Fc1c(Cl)cc(C2CC2c2cnc(-c3ncccn3)nc2)cc1N1CCCC1]

### Example 109: trans-5-(2-(3-Chloro-4-fluoro-5-(pyrrolidin-1-yl)phenyl)cyclopropyl)-2,2′-bipyrimidine (single enantiomer II)

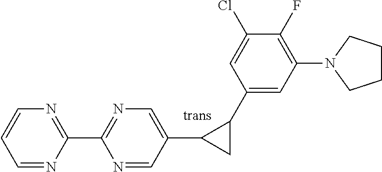 [SMILES: Fc1c(Cl)cc(C2CC2c2cnc(-c3ncccn3)nc2)cc1N1CCCC1]

A mixture of enantiomers of trans-5-(2-(3-chloro-4-fluoro-5-(pyrrolidin-1-yl)phenyl)cyclopropyl)-2,2′-bipyrimidine (120 mg) was separated by SFC (supercritical fluid chromatography) on a CHIRALCEL® OD-H column using liquid CO₂ and MeOH (50:50) to give trans-5-(2-(3-chloro-4-fluoro-5-(pyrrolidin-1-yl)phenyl)cyclopropyl)-2,2′-bipyrimidine (single enantiomer I) as an off-white solid (faster eluting enantiomer, 30 mg, 25%, m/z: 396 [M+H]⁺ observed), and trans-5-(2-(3-chloro-4-fluoro-5-(pyrrolidin-1-yl)phenyl)cyclopropyl)-2,2′-bipyrimidine (single enantiomer II) as an off-white solid (slower eluting enantiomer, 34 mg, 28%, m/z: 396 [M+H]⁺ observed).

### Example 108: trans-5-(2-(3-Chloro-4-fluoro-5-(pyrrolidin-1-yl)phenyl)cyclopropyl)-2,2′-bipyrimidine (single enantiomer I)

m/z: 396 [M+H]⁺ observed. ¹H NMR (400 MHz, DMSO-d₆): δ 9.01 (d, 2H), 8.76 (s, 2H), 7.42 (t, 1H), 6.46-6.44 (m, 1H), 6.37-6.34 (m, 1H), 3.44-3.37 (m, 4H), 2.27-2.20 (m, 1H), 2.18-2.15 (m, 1H), 1.98-1.94 (m, 4H), 1.63-1.54 (m, 2H).

### Example 109: trans-5-(2-(3-Chloro-4-fluoro-5-(pyrrolidin-1-yl)phenyl)cyclopropyl)-2,2′-bipyrimidine (single enantiomer II)

m/z: 396 [M+H]⁺ observed. ¹H NMR (400 MHz, DMSO-d₆): δ 9.01 (d, 2H), 8.76 (s, 2H), 7.42 (t, 1H), 6.46-6.44 (m, 1H), 6.37-6.34 (m, 1H), 3.44-3.37 (m, 4H), 2.27-2.20 (m, 1H), 2.18-2.15 (m, 1H), 1.98-1.94 (m, 4H), 1.63-1.54 (m, 2H).

### Example 110: trans-5-(2-(3-Chloro-4-fluoro-5-((R)-3-methoxypyrrolidin-1-yl)phenyl)cyclopropyl)-2,2′-bipyrimidine (single diastereomer I)

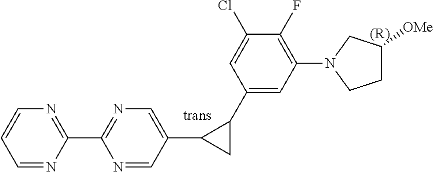

### Example 111: trans-5-(2-(3-Chloro-4-fluoro-5-((R)-3-methoxypyrrolidin-1-yl)phenyl)cyclopropyl)-2,2′-bipyrimidine (single diastereomer II)

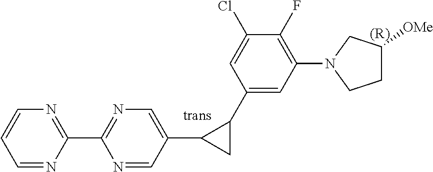

A mixture of diastereomers of trans-5-(2-(3-chloro-4-fluoro-5-((R)-3-methoxypyrrolidin-1-yl)phenyl)cyclopropyl)-2,2′-bipyrimidine (90 mg) was separated by SFC (supercritical fluid chromatography) on a CHIRALCEL® OD-H column using liquid CO₂ and MeOH (50:50) to give trans-5-(2-(3-chloro-4-fluoro-5-((R)-3-methoxypyrrolidin-1-yl)phenyl)cyclopropyl)-2,2′-bipyrimidine (single diastereomer I) as an off-white solid (faster eluting diastereomer, 26 mg, 28%, m/z: 426 [M+H]⁺ observed), and trans-5-(2-(3-chloro-4-fluoro-5-((R)-3-methoxypyrrolidin-1-yl)phenyl)cyclopropyl)-2,2′-bipyrimidine (single diastereomer II) as an off-white solid (slower eluting diastereomer, 22 mg, 24%, m/z: 426 [M+H]⁺ observed).

### Example 110: trans-5-(2-(3-Chloro-4-fluoro-5-((R)-3-methoxypyrrolidin-1-yl)phenyl)cyclopropyl)-2,2′-bipyrimidine (single diastereomer I)

m/z: 426 [M+H]⁺ observed. ¹H NMR (400 MHz, DMSO-d₆): δ 8.99 (d, 2H), 8.83 (s, 2H), 7.62 (t, 1H), 6.66-6.64 (m, 1H), 6.56-6.53 (m, 1H), 4.04-4.01 (m, 1H), 3.61-3.55 (m, 1H), 3.45-3.33 (m, 3H), 3.25 (s, 3H), 2.49-2.41 (m, 1H), 2.37-2.33 (m, 1H), 2.03-1.97 (m, 2H), 1.72-1.62 (m, 2H).

### Example 111: trans-5-(2-(3-Chloro-4-fluoro-5-((R)-3-methoxypyrrolidin-1-yl)phenyl)cyclopropyl)-2,2′-bipyrimidine (single diastereomer II)

m/z: 426 [M+H]⁺ observed. ¹H NMR (400 MHz, DMSO-d₆): δ 8.99 (d, 2H), 8.83 (s, 2H), 7.62 (t, 1H), 6.66-6.64 (m, 1H), 6.56-6.53 (m, 1H), 4.04-4.01 (m, 1H), 3.61-3.55 (m, 1H), 3.45-3.33 (m, 3H), 3.25 (s, 3H), 2.49-2.41 (m, 1H), 2.37-2.33 (m, 1H), 2.03-1.97 (m, 2H), 1.72-1.62 (m, 2H).

### Example 112: trans-5-(2-(3-Fluoro-5-methoxyphenyl)cyclopropyl)-2,2′-bipyrimidine (single enantiomer I)

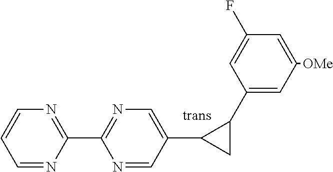 [SMILES: COc1cc(F)cc(C2CC2c2cnc(-c3ncccn3)nc2)c1]

### Example 113: trans-5-(2-(3-Fluoro-5-methoxyphenyl)cyclopropyl)-2,2′-bipyrimidine (single enantiomer II)

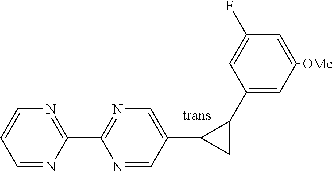 [SMILES: COc1cc(F)cc(C2CC2c2cnc(-c3ncccn3)nc2)c1]

A mixture of enantiomers of trans-5-(2-(3-fluoro-5-methoxyphenyl)cyclopropyl)-2,2′-bipyrimidine (200 mg) was separated by SFC (supercritical fluid chromatography) on a CHIRALCEL® OD-H column using liquid CO₂ and MeOH (55:45) to give trans-5-(2-(3-fluoro-5-methoxyphenyl)cyclopropyl)-2,2′-bipyrimidine (single enantiomer I) as an off-white solid (faster eluting enantiomer, 60 mg, 30%, m/z: 323 [M+H]⁺ observed), and trans-5-(2-(3-fluoro-5-methoxyphenyl)cyclopropyl)-2,2′-bipyrimidine (single enantiomer II) as an off-white solid (slower eluting enantiomer, 60 mg, 30%, m/z: 323 [M+H]⁺ observed).

### Example 112: trans-5-(2-(3-Fluoro-5-methoxyphenyl)cyclopropyl)-2,2′-bipyrimidine (single enantiomer I)

m/z: 323 [M+H]⁺ observed. ¹H NMR (400 MHz, DMSO-d₆): δ 9.01 (d, 2H), 8.77 (s, 2H), 7.42 (t, 1H), 6.53-6.45 (m, 3H), 3.80 (s, 3H), 2.32-2.22 (m, 2H), 1.66-1.62 (m, 2H).

### Example 113: trans-5-(2-(3-Fluoro-5-methoxyphenyl)cyclopropyl)-2,2′-bipyrimidine (single enantiomer II)

m/z: 323 [M+H]⁺ observed. ¹H NMR (400 MHz, DMSO-d₆): δ 9.01 (d, 2H), 8.77 (s, 2H), 7.42 (t, 1H), 6.53-6.45 (m, 3H), 3.80 (s, 3H), 2.32-2.22 (m, 2H), 1.66-1.62 (m, 2H).

### Example 114: trans-5-(2-(4-Chloro-2,3-dimethoxyphenyl)cyclopropyl)-2,2′-bipyrimidine (single enantiomer I)

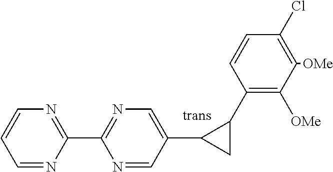 [SMILES: COc1c(Cl)ccc(C2CC2c2cnc(-c3ncccn3)nc2)c1OC]

### Example 115: trans-5-(2-(4-Chloro-2,3-dimethoxyphenyl)cyclopropyl)-2,2′-bipyrimidine (single enantiomer II)

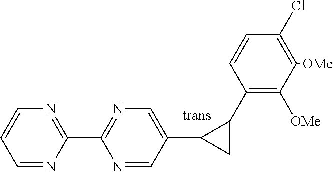 [SMILES: COc1c(Cl)ccc(C2CC2c2cnc(-c3ncccn3)nc2)c1OC]

A mixture of enantiomers of trans-5-(2-(4-chloro-2,3-dimethoxyphenyl)cyclopropyl)-2,2′-bipyrimidine (91 mg) was separated by SFC (supercritical fluid chromatography) on a CHIRALCEL® OD-H column using liquid CO₂ and MeOH (65:35) to give trans-5-(2-(4-chloro-2,3-dimethoxyphenyl)cyclopropyl)-2,2′-bipyrimidine (single enantiomer I) as an off-white solid (faster eluting enantiomer, 17 mg, 18%, m/z: 369 [M+H]⁺ observed), and trans-5-(2-(4-chloro-2,3-dimethoxyphenyl)cyclopropyl)-2,2′-bipyrimidine (single enantiomer II) as an off-white solid (slower eluting enantiomer, 12.5 mg, 13%, m/z: 369 [M+H]⁺ observed).

### Example 114: trans-5-(2-(4-Chloro-2,3-dimethoxyphenyl)cyclopropyl)-2,2′-bipyrimidine (single enantiomer I)

m/z: 369 [M+H]⁺ observed. ¹H NMR (400 MHz, DMSO-d₆): δ 9.02 (d, 2H), 8.83 (s, 2H), 7.43 (t, 1H), 7.10 (d, 1H), 6.67 (d, 1H), 3.91 (s, 3H), 3.86 (s, 3H), 2.59-2.53 (m, 1H), 2.16-2.10 (m, 1H), 1.66-1.57 (m, 2H).

### Example 115: trans-5-(2-(4-Chloro-2,3-dimethoxyphenyl)cyclopropyl)-2,2′-bipyrimidine (single enantiomer II)

m/z: 369 [M+H]⁺ observed. ¹H NMR (400 MHz, DMSO-d₆): δ 9.02 (d, 2H), 8.83 (s, 2H), 7.43 (t, 1H), 7.10 (d, 1H), 6.67 (d, 1H), 3.91 (s, 3H), 3.86 (s, 3H), 2.59-2.53 (m, 1H), 2.16-2.10 (m, 1H), 1.66-1.57 (m, 2H).

### Example 116: trans-5-(2-(3-Chloro-4-fluoro-5-((S)-3-methoxypyrrolidin-1-yl)phenyl)cyclopropyl)-2,2′-bipyrimidine (single diastereomer I)

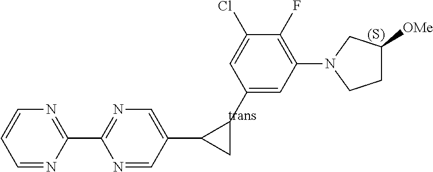

### Example 117: trans-5-(2-(3-Chloro-4-fluoro-5-((S)-3-methoxypyrrolidin-1-yl)phenyl)cyclopropyl)-2,2′-bipyrimidine (single diastereomer II)

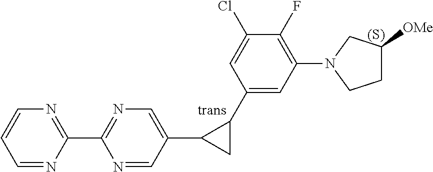

A mixture of diasteromers of trans-5-(2-(3-chloro-4-fluoro-5-((S)-3-methoxypyrrolidin-1-yl)phenyl)cyclopropyl)-2,2′-bipyrimidine (110 mg) was separated by SFC (supercritical fluid chromatography) on a CHIRALCEL® OD-H column using liquid CO₂ and MeOH (50:50) to give trans-5-(2-(3-chloro-4-fluoro-5-((S)-3-methoxypyrrolidin-1-yl)phenyl)cyclopropyl)-2,2′-bipyrimidine (single diastereomer I) as an off-white solid (faster eluting diastereomer, 32 mg, 29%, m/z: 426 [M+H]⁺ observed), and trans-5-(2-(3-chloro-4-fluoro-5-((S)-3-methoxypyrrolidin-1-yl)phenyl)cyclopropyl)-2,2′-bipyrimidine (single diastereomer II) as an off-white solid (slower eluting diastereomer, 26 mg, 23%, m/z: 426 [M+H]⁺ observed).

### Example 116: trans-5-(2-(3-Chloro-4-fluoro-5-((S)-3-methoxypyrrolidin-1-yl)phenyl)cyclopropyl)-2,2′-bipyrimidine (single diastereomer I)

m/z: 426 [M+H]⁺ observed. ¹H NMR (400 MHz, DMSO-d₆): δ 8.99 (d, 2H), 8.83 (s, 2H), 7.63-7.61 (m, 1H), 6.66-6.64 (m, 1H), 6.56-6.53 (m, 1H), 4.04-4.01 (m, 1H), 3.60-3.55 (m, 1H), 3.43-3.34 (m, 3H) , 3.25 (s, 3H), 2.49-2.43 (m, 1H), 2.39-2.32 (m, 1H), 2.03-1.97 (m, 2H), 1.71-1.62 (m, 2H).

### Example 117: trans-5-(2-(3-Chloro-4-fluoro-5-((S)-3-methoxypyrrolidin-1-yl)phenyl)cyclopropyl)-2,2′-bipyrimidine (single diastereomer II)

m/z: 426 [M+H]⁺ observed. ¹H NMR (400 MHz, DMSO-d₆): δ 8.99 (d, 2H), 8.83 (s, 2H), 7.63-7.61 (m, 1H), 6.66-6.64 (m, 1H), 6.56-6.53 (m, 1H), 4.04-4.01 (m, 1H), 3.60-3.55 (m, 1H), 3.43-3.34 (m, 3H) , 3.25 (s, 3H), 2.49-2.43 (m, 1H), 2.39-2.32 (m, 1H), 2.03-1.97 (m, 2H), 1.71-1.62 (m, 2H).

### Example 118: trans-5-(2-(3-Chloro-5-fluorophenyl)cyclopropyl)-2,2′-bipyrimidine (single enantiomer I)

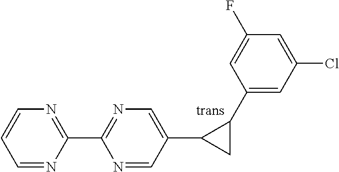 [SMILES: Fc1cc(Cl)cc(C2CC2c2cnc(-c3ncccn3)nc2)c1]

### Example 119: trans-5-(2-(3-Chloro-5-fluorophenyl)cyclopropyl)-2,2′-bipyrimidine (single enantiomer II)

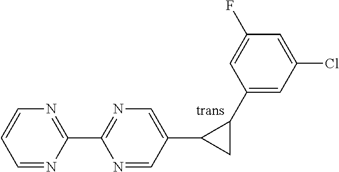 [SMILES: Fc1cc(Cl)cc(C2CC2c2cnc(-c3ncccn3)nc2)c1]

A mixture of enantiomers of trans-5-(2-(3-chloro-5-fluorophenyl)cyclopropyl)-2,2′-bipyrimidine (130 mg) was separated by SFC (supercritical fluid chromatography) on a CHIRALPAK® IC column using liquid CO₂ and 30 mM Methanolic ammonia in EtOH (50:50) to give trans-5-(2-(3-chloro-5-fluorophenyl)cyclopropyl)-2,2′-bipyrimidine (single enantiomer I) as an off-white solid (faster eluting enantiomer, 35 mg, 27%, m/z: 327 [M+H]⁺ observed), and trans-5-(2-(3-chloro-5-fluorophenyl)cyclopropyl)-2,2′-bipyrimidine (single enantiomer II) as an off-white solid (slower eluting enantiomer, 32 mg, 25%, m/z: 327 [M+H]⁺ observed).

### Example 118: trans-5-(2-(3-Chloro-5-fluorophenyl)cyclopropyl)-2,2′-bipyrimidine (single enantiomer I)

m/z: 327 [M+H]⁺ observed. ¹H NMR (400 MHz, DMSO-d₆): δ 9.02 (d, 2H), 8.78 (s, 2H), 7.43 (t, 1H), 6.98-6.95 (m, 2H), 6.79-6.77 (m, 1H), 2.35-2.23 (m, 2H), 1.71-1.62 (m, 2H).

### Example 119: trans-5-(2-(3-Chloro-5-fluorophenyl)cyclopropyl)-2,2′-bipyrimidine (single enantiomer II)

m/z: 327 [M+H]⁺ observed. ¹H NMR (400 MHz, DMSO-d₆): δ 9.02 (d, 2H), 8.78 (s, 2H), 7.43 (t, 1H), 6.98-6.95 (m, 2H), 6.79-6.77 (m, 1H), 2.35-2.23 (m, 2H), 1.71-1.62 (m, 2H).

### Example 120: trans-(3S)-1-(5-(2-([2,2′-Bipyrimidin]-5-yl)cyclopropyl)-3-chloro-2-fluorophenyl)pyrrolidin-3-ol (single diastereomer I)

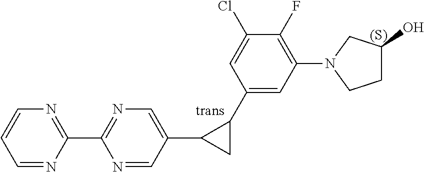

### Example 121: trans-(3S)-1-(5-(2-([2,2′-Bipyrimidin]-5-yl)cyclopropyl)-3-chloro-2-fluorophenyl)pyrrolidin-3-ol (single diastereomer II)

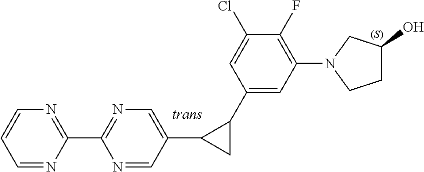

A mixture of diasteromers of trans-(3S)-1-(5-(2-([2,2′-bipyrimidin]-5-yl)cyclopropyl)-3-chloro-2-fluorophenyl)pyrrolidin-3-ol (75 mg) was separated by SFC (supercritical fluid chromatography) on a CHIRALCEL® OD-H column using liquid CO₂ and MeOH (55:45) to give trans-(3S)-1-(5-(2-([2,2′-bipyrimidin]-5-yl)cyclopropyl)-3-chloro-2-fluorophenyl)pyrrolidin-3-ol (single diastereomer I) as an off-white solid (faster eluting diastereomer, 25 mg, 33%, m/z: 412 [M+H]⁺ observed), and trans-(3S)-1-(5-(2-([2,2′-bipyrimidin]-5-yl)cyclopropyl)-3-chloro-2-fluorophenyl)pyrrolidin-3-ol (single diastereomer II) as an off-white solid (slower eluting diastereomer, 25 mg, 33%, m/z: 412 [M+H]⁺ observed).

### Example 120: trans-(3S)-1-(5-(2-([2,2′-Bipyrimidin]-5-yl)cyclopropyl)-3-chloro-2-fluorophenyl)pyrrolidin-3-ol (single diastereomer I)

m/z: 412 [M+H]⁺ observed. ¹H NMR (400 MHz, DMSO-d₆): δ 8.99 (d, 2H), 8.83 (s, 2H), 7.62 (t, 1H), 6.63-6.60 (m, 1H), 6.54-6.51 (m, 1H), 4.94 (br s, 1H), 4.35-4.33 (m, 1H), 3.61-3.57 (m, 1H), 3.55-3.46 (m, 1H), 3.39-3.32 (m, 1H), 3.21-3.17 (m, 1H), 2.52-2.45 (m, 1H), 2.40-2.32 (m, 1H), 2.01-1.91 (m, 1H), 1.87-1.82 (m, 1H), 1.71-1.60 (m, 2H).

### Example 121: trans-(3S)-1-(5-(2-([2,2′-Bipyrimidin]-5-yl)cyclopropyl)-3-chloro-2-fluorophenyl)pyrrolidin-3-ol (single diastereomer II)

m/z: 412 [M+H]⁺ observed. ¹H NMR (400 MHz, DMSO-d₆): δ 8.99 (d, 2H), 8.83 (s, 2H), 7.62 (t, 1H), 6.63-6.60 (m, 1H), 6.54-6.51 (m, 1H), 4.94 (br s, 1H), 4.35-4.33 (m, 1H), 3.61-3.57 (m, 1H), 3.55-3.46 (m, 1H), 3.39-3.32 (m, 1H), 3.21-3.17 (m, 1H), 2.52-2.45 (m, 1H), 2.40-2.32 (m, 1H), 2.01-1.91 (m, 1H), 1.87-1.82 (m, 1H), 1.71-1.60 (m, 2H).

### Example 122: trans-5-(2-(3,4-difluoro-5-(3-methoxyazetidin-1-yl)phenyl)cyclopropyl)-2,2′-bipyrimidine (single enantiomer I)

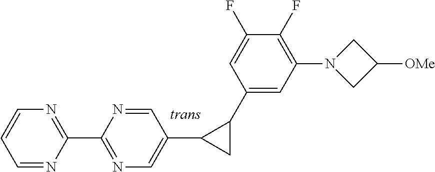 [SMILES: COC1CN(c2cc(C3CC3c3cnc(-c4ncccn4)nc3)cc(F)c2F)C1]

### Example 123: trans-5-(2-(3,4-Difluoro-5-(3-methoxyazetidin-1-yl)phenyl)cyclopropyl)-2,2′-bipyrimidine (single enantiomer II)

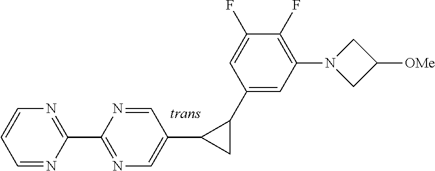 [SMILES: COC1CN(c2cc(C3CC3c3cnc(-c4ncccn4)nc3)cc(F)c2F)C1]

A mixture of enantiomers of trans-5-(2-(3,4-difluoro-5-(3-methoxyazetidin-1-yl)phenyl)cyclopropyl)-2,2′-bipyrimidine (260 mg) was separated by SFC (supercritical fluid chromatography) on a CHIRALCEL® OD-H column using liquid CO₂ and MeOH (60:40) to give trans-5-(2-(3,4-difluoro-5-(3-methoxyazetidin-1-yl)phenyl)cyclopropyl)-2,2′-bipyrimidine (single enantiomer I) as an off-white solid (faster eluting enantiomer, 22 mg, 8%, m/z: 396 [M+H]⁺ observed), and trans-5-(2-(3,4-difluoro-5-(3-methoxyazetidin-1-yl)phenyl)cyclopropyl)-2,2′-bipyrimidine (single enantiomer II) as an off-white solid (slower eluting enantiomer, 32 mg, 12%, m/z: 396 [M+H]⁺ observed).

### Example 122: trans-5-(2-(3,4-Difluoro-5-(3-methoxyazetidin-1-yl)phenyl)cyclopropyl)-2,2′-bipyrimidine (single enantiomer I)

m/z: 396 [M+H]⁺ observed. ¹H NMR (400 MHz, DMSO-d₆): δ 8.98 (d, 2H), 8.82 (s, 2H), 7.62 (t, 1H), 6.60-6.55 (m, 1H), 6.26-6.25 (m, 1H), 4.31-4.28 (m, 1H), 4.19-4.15 (m, 2H), 3.76-3.73 (m, 2H), 3.23 (s, 3H), 2.49-2.45 (m, 1H), 2.44-2.41 (m, 1H), 1.72-1.67 (m, 1H), 1.65-1.60 (m, 1H).

### Example 123: trans-5-(2-(3,4-Difluoro-5-(3-methoxyazetidin-1-yl)phenyl)cyclopropyl)-2,2′-bipyrimidine (single enantiomer II)

m/z: 396 [M+H]⁺ observed. ¹H NMR (400 MHz, DMSO-d₆): δ 8.98 (d, 2H), 8.82 (s, 2H), 7.62 (t, 1H), 6.60-6.55 (m, 1H), 6.26-6.25 (m, 1H), 4.31-4.28 (m, 1H), 4.19-4.15 (m, 2H), 3.76-3.73 (m, 2H), 3.23 (s, 3H), 2.49-2.45 (m, 1H), 2.44-2.41 (m, 1H), 1.72-1.67 (m, 1H), 1.65-1.60 (m, 1H).

### Example 124: trans-2-(5-(2-(3,4-Difluoro-5-methoxyphenyl)cyclopropyl)pyridin-2-yl)pyrimidine (single enantiomer I)

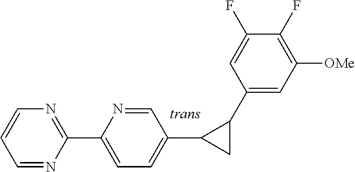 [SMILES: COc1cc(C2CC2c2ccc(-c3ncccn3)nc2)cc(F)c1F]

### Example 125: trans-2-(5-(2-(3,4-Difluoro-5-methoxyphenyl)cyclopropyl)pyridin-2-yl)pyrimidine (single enantiomer II)

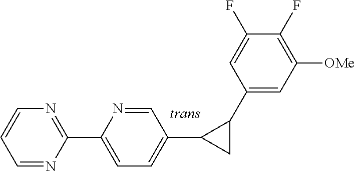 [SMILES: COc1cc(C2CC2c2ccc(-c3ncccn3)nc2)cc(F)c1F]

A mixture of enantiomers of trans-2-chloro-5-(2-(3,4-difluoro-5-methoxyphenyl)cyclopropyl)pyridine (750 mg) was separated by SFC (supercritical fluid chromatography) on a CHIRALPAK® IC column using liquid CO₂ and IPA (70:30) to give trans-2-chloro-5-(2-(3,4-difluoro-5-methoxyphenyl)cyclopropyl)pyridine (single enantiomer I) as a white solid (faster eluting enantiomer, 150 mg, 20%, m/z: 296 [M+H]⁺ observed), and trans-2-chloro-5-(2-(3,4-difluoro-5-methoxyphenyl)cyclopropyl)pyridine (single enantiomer II) as a white solid (slower eluting enantiomer, 150 mg, 20%, m/z: 296 [M+H]⁺ observed). To a solution of trans-2-chloro-5-(2-(3,4-difluoro-5-methoxyphenyl)cyclopropyl)pyridine (single enantiomer I, faster eluting enantiomer, 150 mg, 0.50 mmol) in 3 mL of DMF (3 mL) was added 2-(tributylstannyl)pyrimidine (188 mg, 0.50 mmol), tetraethylammonium chloride (83 mg, 0.50 mmol), and K₂CO₃ (140 mg, 1.01 mmol). The reaction mixture was purged with N₂ gas for 10 min followed by addition of PdCl₂(PPh₃)₂ (35 mg, 0.050 mmol). The mixture was purged with N₂ gas for 10 min. The reaction mixture was stirred at 110° C. for 16 h. The reaction mixture was diluted with water (50 mL) and extracted with EtOAc (2×50 mL). The combined organic layer was washed with saturated aqueous brine solution (50 mL), dried over Na₂SO₄ and evaporated to dryness. The crude was purified by reverse phase HPLC, to afford trans-2-(5-(2-(3,4-difluoro-5-methoxyphenyl)cyclopropyl)pyridin-2-yl)pyrimidine as a white solid (40 mg, 23% yield, m/z: 340 [M+H]⁺ observed). However, chiral purity was observed to be diminished.

The same reaction conditions were utilized with trans-2-chloro-5-(2-(3,4-difluoro-5-methoxyphenyl)cyclopropyl)pyridine (single enantiomer II, slower eluting enantiomer, 150 mg, 0.50 mmol) to afford trans-2-(5-(2-(3,4-difluoro-5-methoxyphenyl)cyclopropyl)pyridin-2-yl)pyrimidine as a white solid (40 mg, 23% yield, m/z: 340 [M+H]⁺ observed). However, chiral purity was observed to be diminished.

A mixture of enantiomers of trans-2-(5-(2-(3,4-difluoro-5-methoxyphenyl)cyclopropyl)pyridin-2-yl)pyrimidine (80 mg) was separated again by SFC (supercritical fluid chromatography) on a CHIRALCEL® OD-H column using liquid CO₂ and MeOH (70:30) to give trans-2-(5-(2-(3,4-difluoro-5-methoxyphenyl)cyclopropyl)pyridin-2-yl)pyrimidine (single enantiomer I) as an off-white solid (faster eluting enantiomer, 20 mg, 25%, m/z: 340 [M+H]⁺ observed), and trans-2-(5-(2-(3,4-difluoro-5-methoxyphenyl)cyclopropyl)pyridin-2-yl)pyrimidine (single enantiomer II) as an off-white solid (slower eluting enantiomer, 17 mg, 21%, m/z: 340 [M+H]⁺ observed).

### Example 124: trans-2-(5-(2-(3,4-Difluoro-5-methoxyphenyl)cyclopropyl)pyridin-2-yl)pyrimidine (single enantiomer I)

m/z: 340 [M+H]⁺ observed. ¹H NMR (400 MHz, DMSO-d₆): δ 8.94 (d, 2H), 8.64 (d, 1H), 8.31 (d, 1H), 7.71-7.68 (m, 1H), 7.53-7.51 (t, 1H), 6.92-6.83 (m, 2H), 3.88 (s, 3H), 2.43-2.39 (m, 2H), 1.68-1.61 (m, 2H).

### Example 125: trans-2-(5-(2-(3,4-Difluoro-5-methoxyphenyl)cyclopropyl)pyridin-2-yl)pyrimidine (single enantiomer II)

m/z: 340 [M+H]⁺ observed. ¹H NMR (400 MHz, DMSO-d₆): δ 8.94 (d, 2H), 8.64 (d, 1H), 8.31 (d, 1H), 7.71-7.68 (m, 1H), 7.53-7.51 (t, 1H), 6.92-6.83 (m, 2H), 3.88 (s, 3H), 2.43-2.39 (m, 2H), 1.68-1.61 (m, 2H).

### Example 126: trans-(3R)-1-(5-(2-([2,2′-Bipyrimidin]-5-yl)cyclopropyl)-3-chloro-2-fluorophenyl)pyrrolidin-3-ol (single diastereomer I)

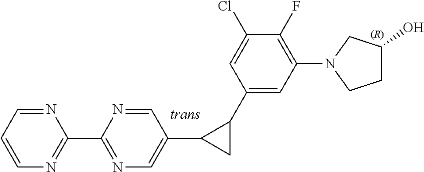

### Example 127: trans-(3R)-1-(5-(2-([2,2′-Bipyrimidin]-5-yl)cyclopropyl)-3-chloro-2-fluorophenyl)pyrrolidin-3-ol (single diastereomer II)

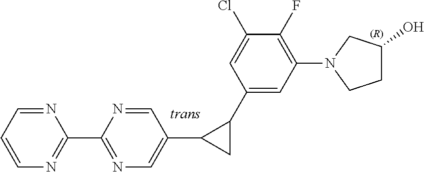

A mixture of diastereomers of trans-(3R)-1-(5-(2-([2,2′-bipyrimidin]-5-yl)cyclopropyl)-3-chloro-2-fluorophenyl)pyrrolidin-3-ol (100 mg) was separated by SFC (supercritical fluid chromatography) on a CHIRALPAK® OD-3 column using liquid CO₂ and MeOH (60:40) to give trans-(3R)-1-(5-(2-([2,2′-bipyrimidin]-5-yl)cyclopropyl)-3-chloro-2-fluorophenyl)pyrrolidin-3-ol (single diastereomer I) as an off-white solid (faster eluting diastereomer, 7 mg, 7% yield, m/z: 412 [M+H]⁺ observed), and trans-(3R)-1-(5-(2-([2,2′-bipyrimidin]-5-yl)cyclopropyl)-3-chloro-2-fluorophenyl)pyrrolidin-3-ol (single diastereomer II) as an off-white solid (slower eluting diastereomer, 7 mg, 7% yield, m/z: 412 [M+H]⁺ observed).

### Example 126: trans-(3R)-1-(5-(2-([2,2′-Bipyrimidin]-5-yl)cyclopropyl)-3-chloro-2-fluorophenyl)pyrrolidin-3-ol (single diastereomer I)

m/z: 412 [M+H]⁺ observed. ¹H NMR (400 MHz, DMSO-d₆): δ 8.99 (d, 2H), 8.83 (s, 2H), 7.62 (t, 1H), 6.65-6.58 (m, 1H), 6.52 (dd, J=7.9, 2.2 Hz, 1H), 4.95 (s, 1H), 4.39-4.30 (m, 1H), 3.6-3.55 (m, 1H), 3.53-3.45 (m, 1H), 3.40-3.33 (m, 1H), 3.22-3.16 (m, 1H), 2.48-2.43 (m, 1H), 2.41-2.34 (m, 1H), 2.02-1.91 (m, 1H), 1.88-1.79 (m, 1H), 1.73-1.59 (m, 2H).

### Example 127: trans-(3R)-1-(5-(2-([2,2′-Bipyrimidin]-5-yl)cyclopropyl)-3-chloro-2-fluorophenyl)pyrrolidin-3-ol (single diastereomer II)

m/z: 412 [M+H]⁺ observed. ¹H NMR (400 MHz, DMSO-d₆): δ 8.99 (d, 2H), 8.83 (s, 2H), 7.62 (t, 1H), 6.65-6.58 (m, 1H), 6.52 (dd, J=7.9, 2.2 Hz, 1H), 4.95 (s, 1H), 4.39-4.30 (m, 1H), 3.6-3.55 (m, 1H), 3.53-3.45 (m, 1H), 3.40-3.33 (m, 1H), 3.22-3.16 (m, 1H), 2.48-2.43 (m, 1H), 2.41-2.34 (m, 1H), 2.02-1.91 (m, 1H), 1.88-1.79 (m, 1H), 1.73-1.59 (m, 2H).

### Example 128: trans-5-(2-(3-(Cyclopropylmethoxy)-4,5-difluorophenyl)cyclopropyl)-2,2′-bipyrimidine (single enantiomer I)

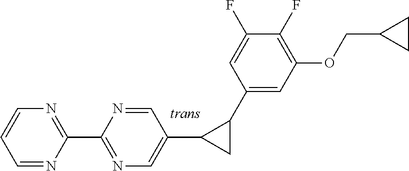 [SMILES: Fc1cc(C2CC2c2cnc(-c3ncccn3)nc2)cc(OCC2CC2)c1F]

### Example 129: trans-5-(2-(3-(Cyclopropylmethoxy)-4,5-difluorophenyl)cyclopropyl)-2,2′-bipyrimidine (single enantiomer II)

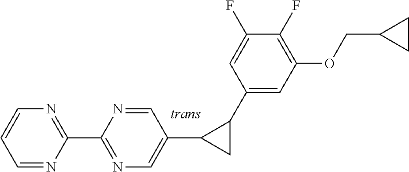 [SMILES: Fc1cc(C2CC2c2cnc(-c3ncccn3)nc2)cc(OCC2CC2)c1F]

A mixture of enantiomers of trans-5-(2-(3-(cyclopropylmethoxy)-4,5-difluorophenyl)cyclopropyl)-2,2′-bipyrimidine (240 mg) was separated by SFC (supercritical fluid chromatography) on a CHIRALCEL® OD-H column using liquid CO₂ and MeOH (60:40) to give trans-5-(2-(3-(cyclopropylmethoxy)-4,5-difluorophenyl)cyclopropyl)-2,2′-bipyrimidine (single enantiomer I) as an off-white solid (faster eluting enantiomer, 45 mg, 18%, m/z: 381 [M+H]⁺ observed), and trans-5-(2-(3-(cyclopropylmethoxy)-4,5-difluorophenyl)cyclopropyl)-2,2′-bipyrimidine (single enantiomer II) as an off-white solid (slower eluting enantiomer, 42 mg, 17%, m/z: 381 [M+H]⁺ observed).

### Example 128: trans-5-(2-(3-(Cyclopropylmethoxy)-4,5-difluorophenyl)cyclopropyl)-2,2′-bipyrimidine (single enantiomer I)

m/z: 381 [M+H]⁺ observed. ¹H NMR (400 MHz, CDCl₃): δ 9.02-9.01 (d, 2H), 8.76 (s, 2H), 7.44-7.41 (t, 1H), 6.58-6.52 (m, 2H), 3.90-3.89 (d, 2H), 2.29-2.26 (m, 1H), 2.20-2.15 (m, 1H), 1.63-1.55 (m, 2H), 1.32-1.28 (m, 1H), 0.69-0.64 (m, 2H), 0.39-0.35 (m, 2H).

### Example 129: trans-5-(2-(3-(Cyclopropylmethoxy)-4,5-difluorophenyl)cyclopropyl)-2,2′-bipyrimidine (single enantiomer II)

m/z: 381 [M+H]⁺ observed. ¹H NMR (400 MHz, CDCl₃): δ 9.02-9.01 (d, 2H), 8.76 (s, 2H), 7.44-7.41 (t, 1H), 6.58-6.52 (m, 2H), 3.90-3.89 (d, 2H), 2.29-2.26 (m, 1H), 2.20-2.15 (m, 1H), 1.63-1.55 (m, 2H), 1.32-1.28 (m, 1H), 0.69-0.64 (m, 2H), 0.39-0.35 (m, 2H).

### Example 130: trans-5-(2-(3,4-Difluoro-5-(3-methoxypropoxy)phenyl)cyclopropyl)-2,2′-bipyrimidine (single enantiomer I)

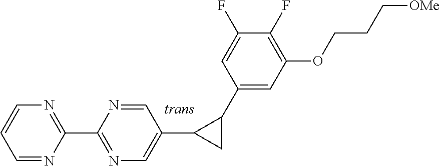 [SMILES: COCCCOc1cc(C2CC2c2cnc(-c3ncccn3)nc2)cc(F)c1F]

### Example 131: trans-5-(2-(3,4-Difluoro-5-(3-methoxypropoxy)phenyl)cyclopropyl)-2,2′-bipyrimidine (single enantiomer II)

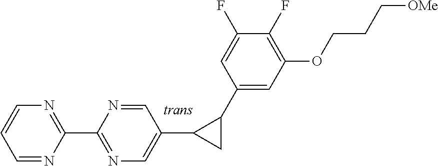 [SMILES: COCCCOc1cc(C2CC2c2cnc(-c3ncccn3)nc2)cc(F)c1F]

A mixture of enantiomers of trans-5-(2-(3,4-difluoro-5-(3-methoxypropoxy)phenyl)cyclopropyl)-2,2′-bipyrimidine (210 mg) was separated by SFC (supercritical fluid chromatography) on a CHIRALCEL® OD-H column using liquid CO₂ and MeOH (55:45) to give trans-5-(2-(3,4-difluoro-5-(3-methoxypropoxy)phenyl)cyclopropyl)-2,2′-bipyrimidine (single enantiomer I) as an off-white solid (faster eluting enantiomer, 42 mg, 20%, m/z: 399 [M+H]⁺ observed), and trans-5-(2-(3,4-difluoro-5-(3-methoxypropoxy)phenyl)cyclopropyl)-2,2′-bipyrimidine (single enantiomer II) as an off-white solid (slower eluting enantiomer, 45 mg, 21%, m/z: 399 [M+H]⁺ observed).

### Example 130: trans-5-(2-(3,4-Difluoro-5-(3-methoxypropoxy)phenyl)cyclopropyl)-2,2′-bipyrimidine (single enantiomer I)

m/z: 399 [M+H]⁺ observed. ¹H NMR (400 MHz, DMSO-d₆): δ 8.99-8.98 (d, 2H), 8.84 (s, 2H), 7.62(t, 1H), 6.94-6.85 (m, 2H), 4.15 (t, 2H), 3.47 (t, 2H), 3.27 (s, 3H), 2.53-2.51 (m, 1H), 2.41-2.36 (m, 1H), 2.00-1.94 (m, 2H), 1.76-1.71 (m, 2H).

### Example 131: trans-5-(2-(3,4-Difluoro-5-(3-methoxypropoxy)phenyl)cyclopropyl)-2,2′-bipyrimidine (single enantiomer II)

m/z: 399 [M+H]⁺ observed. ¹H NMR (400 MHz, DMSO-d₆): δ 8.99-8.98 (d, 2H), 8.84 (s, 2H), 7.62(t, 1H), 6.94-6.85 (m, 2H), 4.15 (t, 2H), 3.47 (t, 2H), 3.27 (s, 3H), 2.53-2.51 (m, 1H), 2.41-2.36 (m, 1H), 2.00-1.94 (m, 2H), 1.76-1.71 (m, 2H).

### Example 132: trans-5-(2-(2,4-Dichloro-3-methoxyphenyl)cyclopropyl)-2,2′-bipyrimidine (single enantiomer I)

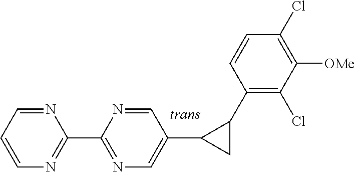 [SMILES: COc1c(Cl)ccc(C2CC2c2cnc(-c3ncccn3)nc2)c1Cl]

### Example 133: trans-5-(2-(2,4-Dichloro-3-methoxyphenyl)cyclopropyl)-2,2′-bipyrimidine (single enantiomer II)

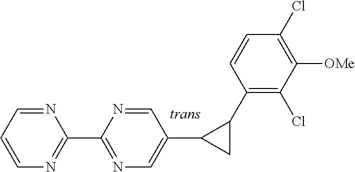 [SMILES: COc1c(Cl)ccc(C2CC2c2cnc(-c3ncccn3)nc2)c1Cl]

A mixture of enantiomers of trans-5-(2-(2,4-dichloro-3-methoxyphenyl)cyclopropyl)-2,2′-bipyrimidine (70 mg) was separated by SFC (supercritical fluid chromatography) on a CHIRALCEL® OD-H column using liquid CO₂ and MeOH (60:40) to give trans-5-(2-(2,4-dichloro-3-methoxyphenyl)cyclopropyl)-2,2′-bipyrimidine (single enantiomer I) as an off-white solid (faster eluting enantiomer, 15 mg, 21%, m/z: 373 [M+H]⁺ observed), and trans-5-(2-(2,4-dichloro-3-methoxyphenyl)cyclopropyl)-2,2′-bipyrimidine (single enantiomer II) as an off-white solid (slower eluting enantiomer, 15 mg, 21%, m/z: 373 [M+H]⁺ observed).

### Example 132: trans-5-(2-(2,4-Dichloro-3-methoxyphenyl)cyclopropyl)-2,2′-bipyrimidine (single enantiomer I)

m/z: 373 [M+H]⁺ observed. ¹H NMR (400 MHz, DMSO-d₆): δ 9.02 (d, 2H), 8.86 (s, 2H), 7.42 (t, 1H), 7.28 (d, 1H), 6.87 (d, 1H), 3.91 (s, 3H), 2.55-2.53 (m, 1H), 2.11-2.09 (m, 1H), 1.70-1.64 (m, 2H).

### Example 133: trans-5-(2-(2,4-Dichloro-3-methoxyphenyl)cyclopropyl)-2,2′-bipyrimidine (single enantiomer II)

m/z: 373 [M+H]⁺ observed. ¹H NMR (400 MHz, DMSO-d₆): δ 9.02 (d, 2H), 8.86 (s, 2H), 7.42 (t, 1H), 7.28 (d, 1H), 6.87 (d, 1H), 3.91 (s, 3H), 2.55-2.53 (m, 1H), 2.11-2.09 (m, 1H), 1.70-1.64 (m, 2H).

### Example 134: trans-5-(2-(3,4-Difluoro-5-(2-methoxyethoxy)phenyl)cyclopropyl)-2,2′-bipyrimidine (single enantiomer I)

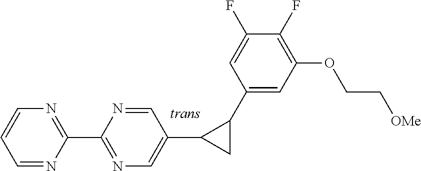 [SMILES: COCCOc1cc(C2CC2c2cnc(-c3ncccn3)nc2)cc(F)c1F]

### Example 135: trans-5-(2-(3,4-Difluoro-5-(2-methoxyethoxy)phenyl)cyclopropyl)-2,2′-bipyrimidine (single enantiomer II)

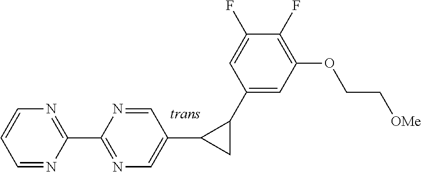 [SMILES: COCCOc1cc(C2CC2c2cnc(-c3ncccn3)nc2)cc(F)c1F]

A mixture of enantiomers of trans-5-(2-(3,4-difluoro-5-(2-methoxyethoxy)phenyl)cyclopropyl)-2,2′-bipyrimidine (110 mg) was separated by SFC (supercritical fluid chromatography) on a CHIRALCEL® OD-H column using liquid CO₂ and MeOH (55:45) to give trans-5-(2-(3,4-difluoro-5-(2-methoxyethoxy)phenyl)cyclopropyl)-2,2′-bipyrimidine (single enantiomer I) as an off-white solid (faster eluting enantiomer, 30 mg, 27%, m/z: 385 [M+H]⁺ observed), and trans-5-(2-(3,4-difluoro-5-(2-methoxyethoxy)phenyl)cyclopropyl)-2,2′-bipyrimidine (single enantiomer II) as an off-white solid (slower eluting enantiomer, 27 mg, 24%, m/z: 385 [M+H]⁺ observed).

### Example 134: trans-5-(2-(3,4-Difluoro-5-(2-methoxyethoxy)phenyl)cyclopropyl)-2,2′-bipyrimidine (single enantiomer I)

m/z: 385 [M+H]⁺ observed.; ¹H NMR (400 MHz, DMSO-d₆): δ 8.98 (d, 2H), 8.84 (s, 2H), 7.62 (t, 1H), 6.94-6.86 (m, 2H), 4.23 (t, 2H), 3.69-3.67 (m, 2H), 3.31 (s, 3H), 2.67-2.51 (m, 1H), 2.41-2.37 (m, 1H), 1.77-1.66 (m, 2H).

### Example 135: trans-5-(2-(3,4-Difluoro-5-(2-methoxyethoxy)phenyl)cyclopropyl)-2,2′-bipyrimidine (single enantiomer II)

m/z: 385 [M+H]⁺ observed.; ¹H NMR (400 MHz, DMSO-d₆): δ 8.98 (d, 2H), 8.84 (s, 2H), 7.62 (t, 1H), 6.94-6.86 (m, 2H), 4.23 (t, 2H), 3.69-3.67 (m, 2H), 3.31 (s, 3H), 2.67-2.51 (m, 1H), 2.41-2.37 (m, 1H), 1.77-1.66 (m, 2H).

### Example 136: trans-2-(5-(2-([2,2′-Bipyrimidin]-5-yl)cyclopropyl)-2,3-difluorophenyl)-7-oxa-2-azaspiro[3.5]nonane (single enantiomer I)

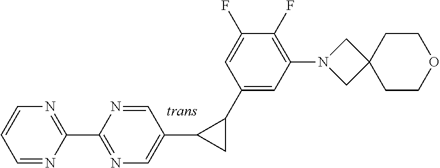 [SMILES: Fc1cc(C2CC2c2cnc(-c3ncccn3)nc2)cc(N2CC3(CCOCC3)C2)c1F]

### Example 137: trans-2-(5-(2-([2,2′-Bipyrimidin]-5-yl)cyclopropyl)-2,3-difluorophenyl)-7-oxa-2-azaspiro[3.5]nonane (single enantiomer II)

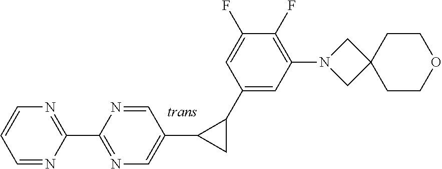 [SMILES: Fc1cc(C2CC2c2cnc(-c3ncccn3)nc2)cc(N2CC3(CCOCC3)C2)c1F]

A mixture of enantiomers of trans-2-(5-(2-([2,2′-bipyrimidin]-5-yl)cyclopropyl)-2,3-difluorophenyl)-7-oxa-2-azaspiro[3.5]nonane (90 mg) was separated by SFC (supercritical fluid chromatography) on a CHIRALCEL® OD-H column using liquid CO₂ and MeOH (55:45) to give trans-2-(5-(2-([2,2′-bipyrimidin]-5-yl)cyclopropyl)-2,3-difluorophenyl)-7-oxa-2-azaspiro[3.5]nonane (single enantiomer I) as an off-white solid (faster eluting enantiomer, 35 mg, 38%, m/z: 436 [M+H]⁺ observed), and trans-2-(5-(2-([2,2′-bipyrimidin]-5-yl)cyclopropyl)-2,3-difluorophenyl)-7-oxa-2-azaspiro[3.5]nonane (single enantiomer II) as an off-white solid (slower eluting enantiomer, 33 mg, 36%, m/z: 436 [M+H]⁺ observed).

### Example 136: trans-2-(5-(2-([2,2′-Bipyrimidin]-5-yl)cyclopropyl)-2,3-difluorophenyl)-7-oxa-2-azaspiro[3.5]nonane (single enantiomer I)

m/z: 436 [M+H]⁺ observed. ¹H NMR (400 MHz, DMSO-d₆): δ 8.98 (d, 2 H), 8.82 (s, 2H), 7.62 (t, 1 H), 6.57-6.52 (m, 1 H), 6.23 (d, 1 H), 3.75 (s, 4 H), 3.54 (t, 4H), 2.49-2.41 (m, 1 H), 2.35-2.30 (m, 1 H), 1.75-1.69 (m, 5H), 1.68-1.61 (m, 1H).

### Example 137: trans-2-(5-(2-([2,2′-Bipyrimidin]-5-yl)cyclopropyl)-2,3-difluorophenyl)-7-oxa-2-azaspiro[3.5]nonane (single enantiomer II)

m/z: 436 [M+H]⁺ observed. ¹H NMR (400 MHz, DMSO-d₆): δ 8.98 (d, 2 H), 8.82 (s, 2H), 7.62 (t, 1 H), 6.57-6.52 (m, 1 H), 6.23 (d, 1 H), 3.75 (s, 4 H), 3.54 (t, 4H), 2.49-2.41 (m, 1 H), 2.35-2.30 (m, 1 H), 1.75-1.69 (m, 5H), 1.68-1.61 (m, 1H).

### Example 138: trans-5-(2-(3-(3,3-Dimethylazetidin-1-yl)-4,5-difluorophenyl)cyclopropyl)-2,2′-bipyrimidine (single enantiomer I)

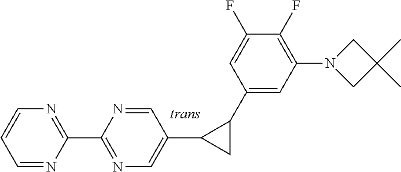 [SMILES: CC1(C)CN(c2cc(C3CC3c3cnc(-c4ncccn4)nc3)cc(F)c2F)C1]

### Example 139: trans-5-(2-(3-(3,3-Dimethylazetidin-1-yl)-4,5-difluorophenyl)cyclopropyl)-2,2′-bipyrimidine (single enantiomer II)

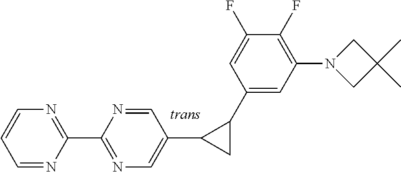 [SMILES: CC1(C)CN(c2cc(C3CC3c3cnc(-c4ncccn4)nc3)cc(F)c2F)C1]

A mixture of enantiomers of trans-5-(2-(3-(3,3-dimethylazetidin-1-yl)-4,5-difluorophenyl)cyclopropyl)-2,2′-bipyrimidine (150 mg) was separated by SFC (supercritical fluid chromatography) on a CHIRALPAK® AD-H column using liquid CO₂ and MeOH (55:45) to give trans-5-(2-(3-(3,3-dimethylazetidin-1-yl)-4,5-difluorophenyl)cyclopropyl)-2,2′-bipyrimidine (single enantiomer I) as an off-white solid (faster eluting enantiomer, 60 mg, 40%, m/z: 394 [M+H]⁺ observed), and trans-5-(2-(3-(3,3-dimethylazetidin-1-yl)-4,5-difluorophenyl)cyclopropyl)-2,2′-bipyrimidine (single enantiomer II) as an off-white solid (slower eluting enantiomer, 59 mg, 39%, m/z: 394 [M+H]⁺ observed).

### Example 138: trans-5-(2-(3-(3,3-Dimethylazetidin-1-yl)-4,5-difluorophenyl)cyclopropyl)-2,2′-bipyrimidine (single enantiomer I)

m/z: 394 [M+H]⁺ observed. ¹H NMR (400 MHz, DMSO-d₆): δ 8.99 (d, 2 H), 8.82 (s, 2 H), 7.62 (t, 1 H), 6.55-6.51 (m, 1 H), 6.20 (d, 1 H), 3.66 (s, 4 H), 2.45-2.41 (m, 1 H), 2.35-2.30 (m, 1 H), 1.71-1.66 (m, 1 H), 1.63-1.58 (m, 1 H), 1.27 (s, 6 H).

### Example 139: trans-5-(2-(3-(3,3-Dimethylazetidin-1-yl)-4,5-difluorophenyl)cyclopropyl)-2,2′-bipyrimidine (single enantiomer II)

m/z: 394 [M+H]⁺ observed. ¹H NMR (400 MHz, DMSO-d₆): δ 8.99 (d, 2 H), 8.82 (s, 2 H), 7.62 (t, 1 H), 6.55-6.51 (m, 1 H), 6.20 (d, 1 H), 3.66 (s, 4 H), 2.45-2.41 (m, 1 H), 2.35-2.30 (m, 1 H), 1.71-1.66 (m, 1 H), 1.63-1.58 (m, 1 H), 1.27 (s, 6 H).

### Example 140: trans-6-(5-(2-(4-Fluoro-3-methoxyphenyl)cyclopropyl)-[2,2′-bipyrimidin]-4-yl)-2-methylbenzoidlthiazole (single enantiomer I)

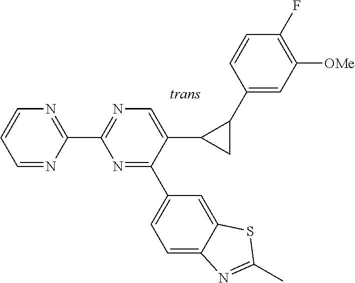 [SMILES: COc1cc(C2CC2c2cnc(-c3ncccn3)nc2-c2ccc3nc(C)sc3c2)ccc1F]

### Example 141: trans-6-(5-(2-(4-Fluoro-3-methoxyphenyl)cyclopropyl)-[2,2′-bipyrimidin]-4-yl)-2-methylbenzoklithiazole (single enantiomer II)

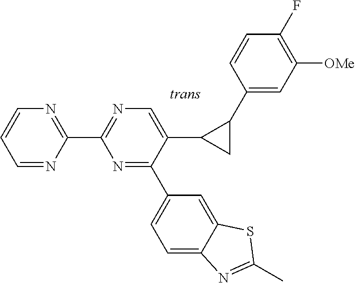 [SMILES: COc1cc(C2CC2c2cnc(-c3ncccn3)nc2-c2ccc3nc(C)sc3c2)ccc1F]

### (E)-2-(4-Fluoro-3-methoxystyryl)-4,4,5,5-tetramethyl-1,3,2-dioxaborolane

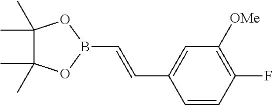 [SMILES: COc1cc(/C=C/B2OC(C)(C)C(C)(C)O2)ccc1F]

To a solution of 4-bromo-1-fluoro-2-methoxybenzene (4 g, 20 mmol) in toluene (20 mL) were added triethylamine (8.2 mL, 59 mmol) and 4,4,5,5-tetramethyl-2-vinyl-1,3,2-dioxaborolane (6 g, 39 mmol) at rt, and the mixture was purged with N₂ gas for 10 min. To this bis(tri-tert-butylphosphine)palladium (100 mg, 0.19 mmol) was added and the mixture was purged with N₂ gas for 10 min. The reaction mixture was heated at 120° C. for 16 h in a sealed tube. The mixture was cooled to rt, poured into ice water (200 mL), and extracted with EtOAc (2×300 mL). The combined organic phase was washed with saturated aqueous brine solution (100 mL), dried with anhydrous sodium sulfate, and concentrated under reduced pressure. The residue was purified by normal phase SiO₂ chromatography (0-10% EtOAc/petroleum ether) to give (E)-2-(4-fluoro-3-methoxystyryl)-4,4,5,5-tetramethyl-1,3,2-dioxaborolane as a white solid (2.6 g, 48% yield, m/z: 279 [M+H]⁺ observed).

### (E)-2,4-Dichloro-5-(4-fluoro-3-methoxystyryl)pyrimidine

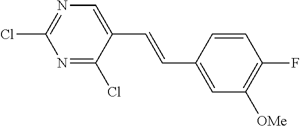 [SMILES: COc1cc(/C=C/c2cnc(Cl)nc2Cl)ccc1F]

To a solution (E)-2-(4-fluoro-3-methoxystyryl)-4,4,5,5-tetramethyl-1,3,2-dioxaborolane (2.5 g, 9.0 mmol) in 1,4-dioxane-water (1:1, 25 mL) was added 2,4-dichloro-5-iodopyrimidine (3.7 g, 13.5 mmol) and K₂CO₃ (2.4 g, 17.4 mmol) at rt, and the mixture was degassed with N₂ gas for 10 min. Tetrakis(triphenylphosphine)palladium(0) (520 mg, 0.45 mmol) was added and the mixture was degassed with N₂ gas for 10 min. The mixture was heated at 90° C. for 16 h in a sealed tube. The reaction mixture was cooled to rt, diluted with water (200 mL), and extracted with EtOAc (2×200 mL). The combined organic phase was washed with saturated aqueous brine solution (200 mL), dried over anhydrous sodium sulfate, filtered, and evaporated under reduced pressure. The residue was purified by normal phase SiO₂ chromatography (0-30% EtOAc/petroleum ether to give (E)-2,4-dichloro-5-(4-fluoro-3-methoxystyryl)pyrimidine as a yellow solid (0.90 g, 33% yield, m/z: 299 [M+H]⁺ observed). ¹H NMR (400 MHz, CDCl₃): δ 8.79 (d, 1H), 7.16-7.03 (m, 5H), 3.96 (s, 3H).

### 2-Methyl-6-(4,4,5,5-tetramethyl-1,3,2-dioxaborolan-2-yl)benzo[d]thiazole

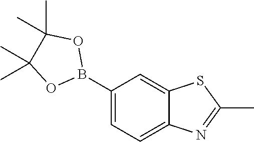

To a solution of 6-bromo-2-methylbenzo[d]thiazole (2.0 g, 8.8 mmol) in DMF (20 mL) was added bis(pinacolato)diboron (6.6 g, 26 mmol) and KOAc (2.5 g, 25 mmol) at rt, and the mixture was degassed with N₂ gas for 10 min. Bis(triphenylphosphine)palladium(II) dichloride (360 mg, 0.51 mmol) was added and the mixture was degassed with N₂ gas for 10 min. The reaction mixture was heated at 120° C. for 16 h in a sealed tube. The mixture was cooled to rt, diluted with water (200 mL), and extracted with EtOAc (2×200 mL). The combined organic phase was washed with saturated aqueous brine solution (200 mL), dried over anhydrous sodium sulfate, filtered, and evaporated under reduced pressure. The residue was purified by normal phase SiO₂ chromatography (0-20% EtOAc/petroleum ether) to give 2-methyl-6-(4,4,5,5-tetramethyl-1,3,2-dioxaborolan-2-yl)benzo[d]thiazole as a white solid (1.2 g, 50% yield, m/z: 276 [M+H]⁺ observed).

### (E)-6-(2-Chloro-5-(4-fluoro-3-methoxystyryl)pyrimidin-4-yl)-2-methylbenzo[d]thiazole

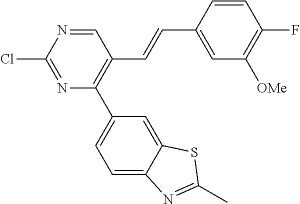 [SMILES: COc1cc(/C=C/c2cnc(Cl)nc2-c2ccc3nc(C)sc3c2)ccc1F]

To a solution of (E)-2,4-dichloro-5-(4-fluoro-3-methoxystyryl)pyrimidine (1.0 g, 3.3 mmol) in THF/H₂O (1:1, 10 mL) was added 2-methyl-6-(4,4,5,5-tetramethyl-1,3,2-dioxaborolan-2-yl)benzo[d]thiazole (1.0 g, 3.7 mmol) and K₂CO₃ (1.4 g, 10 mmol) at rt, and the mixture was purged with N₂ gas for 10 min. Tetrakis(triphenylphosphine)palladium(0) (155 mg, 0.134 mmol) was added and the mixture was degassed with N₂ gas for 10 min. The reaction mixture was heated at 90° C. for 16 h in a sealed tube. The mixture was cooled to rt, diluted with water (200 mL), and extracted with EtOAc (2×200 mL). The combined organic phase was washed with saturated aqueous brine solution (200 mL), dried over anhydrous sodium sulfate, filtered, and evaporated under reduced pressure. The residue was purified by normal phase SiO₂ chromatography (0-20% EtOAc/petroleum ether) to give (E)-6-(2-chloro-5-(4-fluoro-3-methoxystyryl)pyrimidin-4-yl)-2-methylbenzo[d] thiazole as a pale yellow solid (0.60 g, 43% yield, m/z: 412 [M+H]⁺ observed). ¹H NMR (400 MHz, CDCl₃): δ 8.76 (s, 1H), 8.19 (d, 1H), 7.95-7.93 (m, 1H), 7.70-7.68 (m, 1H), 7.03-6.82 (m, 5H), 3.78 (s, 3H), 2.79 (s, 3H).

### trans-6-(2-Chloro-5-(2-(4-fluoro-3-methoxyphenyl)cyclopropyl)pyrimidin-4-yl)-2-methylbenzo[d]thiazole

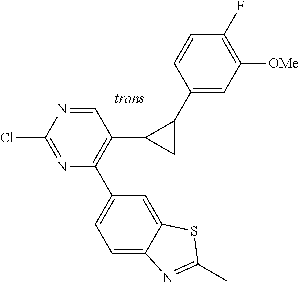 [SMILES: COc1cc(C2CC2c2cnc(Cl)nc2-c2ccc3nc(C)sc3c2)ccc1F]

To a solution of (E)-6-(2-chloro-5-(4-fluoro-3-methoxystyryl)pyrimidin-4-yl)-2-methylbenzo[d]thiazole (0.80 g, 1.9 mmol) in CH₂Cl₂ (10 mL) at −20° C. was added Pd₃(OAc)₆ (130 mg, 0.19 mmol) and freshly prepared ethereal diazomethane [prepared from N-methyl-N-nitroso urea (4.0 g, 39 mmol), KOH solution (50% aqueous solution, 40 mL) and Et₂O (40 mL) at 0° C.]. The reaction mixture was stirred at 0-5° C. for 16 h. The mixture was filtered through a CELITE® pad and filtrate concentrated under reduced pressure. The residue was dissolved in CH₂Cl₂ (10 mL) at −20° C. and Pd₃(OAc)₆ (130 mg, 0.19 mmol) was added, followed by freshly prepared ethereal diazomethane [prepared from N-methyl-N-nitroso urea (4.0 g, 39 mmol), KOH solution (50% aqueous solution, 40 mL) and Et₂O (40 mL) at 0° C.]. The reaction mixture was stirred at 0-5° C. for 16 h. The mixture was filtered through a CELITE® pad and filtrate concentrated under reduced pressure. The residue was purified by normal phase SiO₂ chromatography (0-20% EtOAc/petroleum ether) to give trans-6-(2-chloro-5-(2-(4-fluoro-3-methoxyphenyl)cyclopropyl)pyrimidin-4-yl)-2-methylbenzo[d]thiazole as a pale yellow solid (0.30 g, 36% yield, m/z: 426 [M+H]⁺ observed). ¹H NMR (400 MHz, CDCl₃): δ 8.44 (s, 1H), 8.11 (d, 1H), 7.91-7.89 (m, 1H), 7.82-7.79 (m, 1H), 6.99-6.94 (m, 1H), 6.52-6.43 (m, 2H), 3.76 (s, 3H), 2.84 (s, 3H), 2.23-2.15 (m, 1H), 2.06-2.00 (m, 1H), 1.63-1.51 (m, 2H).

### trans-6-(5-(2-(4-Fluoro-3-methoxyphenyl)cyclopropyl)-[2,2′-bipyrimidin]4-yl)-2-methylbenzo[d]thiazole

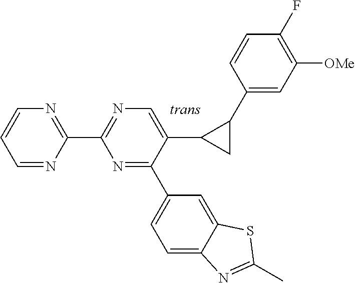 [SMILES: COc1cc(C2CC2c2cnc(-c3ncccn3)nc2-c2ccc3nc(C)sc3c2)ccc1F]

To a solution of trans-6-(2-chloro-5-(2-(4-fluoro-3-methoxyphenyl)cyclopropyl)pyrimidin-4-yl)-2-methylbenzo[d]thiazole (290 mg, 0.68 mmol) in DMF (10 mL) was added 2-(tributylstannyl)pyrimidine (0.3 mL, 0.95 mmol), tetraethylammonium chloride (105 mg, 0.63 mmol), and K₂CO₃ (176 mg, 1.27 mmol) at rt, and the mixture was purged with N₂ gas for 10 min. Bis(triphenylphosphine)palladium(II) dichloride (45 mg, 0.064 mmol) was added and the mixture was purged with N₂ gas for 10 min. The reaction mixture was stirred at 110 ° C. for 16 h. The mixture was cooled to rt, diluted with water (100 mL), and extracted with EtOAc (2×200 mL). The combined organic phase was washed with saturated aqueous brine solution (100 mL), dried over anhydrous sodium sulfate, filtered, and evaporated under reduced pressure. The residue was purified by purified by reverse phase HPLC to give trans-6-(5-(2-(4-fluoro-3-methoxyphenyl)cyclopropyl)-[2,2′-bipyrimidin]-4-yl)-2-methylbenzo[d]thiazole as an off-white gum (80 mg, 25% yield, m/z: 470 [M+H]⁺ observed). ¹H NMR (400 MHz, DMSO-d₆): δ 9.01 (d, 2H), 8.91 (s, 1H), 8.30 (s, 1H), 7.92-7.84 (m, 2H), 7.67-7.63 (m, 1H), 7.10-7.03 (m, 1H), 6.88-6.85 (m, 1H), 6.65-6.61 (m, 1H), 3.73 (s, 3H), 2.83 (s, 3H), 2.32-2.27 (m, 2H), 1.87-1.86 (m, 1H), 1.57-1.55 (m, 1H).

### Example 140: trans-6-(5-(2-(4-Fluoro-3-methoxyphenyl)cyclopropyl)-[2,2′-bipyrimidin]-4-yl)-2-methylbenzo[d]thiazole (single enantiomer I)

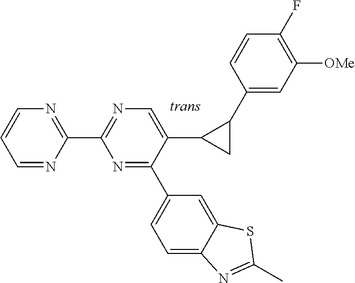 [SMILES: COc1cc(C2CC2c2cnc(-c3ncccn3)nc2-c2ccc3nc(C)sc3c2)ccc1F]

### Example 141: trans-6-(5-(2-(4-Fluoro-3-methoxyphenyl)cyclopropyl)-[2,2′-bipyrimidin]-4-yl)-2-methylbenzo[d]thiazole (single enantiomer II)

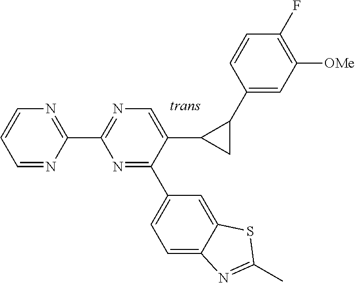 [SMILES: COc1cc(C2CC2c2cnc(-c3ncccn3)nc2-c2ccc3nc(C)sc3c2)ccc1F]

A mixture of enantiomers (80 mg) was separated by HPLC on a CHIRALCEL® OJ-H column using n-hexane and EtOH (35:65) to give trans-6-(5-(2-(4-fluoro-3-methoxyphenyl)cyclopropyl)-[2,2′-bipyrimidin]-4-yl)-2-methylbenzo[d]thiazole (single enantiomer I) as an off-white solid (faster eluting enantiomer, 18 mg, 23%, m/z: 470 [M+H]⁺ observed), and trans-6-(5-(2-(4-fluoro-3-methoxyphenyl)cyclopropyl)-[2,2′-bipyrimidin]-4-yl)-2-methylbenzo[d]thiazole (single enantiomer II) as an off-white solid (slower eluting enantiomer, 16 mg, 20%, m/z: 470 [M+H]+ observed).

### Example 140: trans-6-(5-(2-(4-Fluoro-3-methoxyphenyl)cyclopropyl)-[2,2′-bipyrimidin]-4-yl)-2-methylbenzo[d]thiazole (single enantiomer I)

m/z: 470 [M+H]⁺ observed. ¹H NMR (400 MHz, DMSO-d₆): δ 9.01 (d, 2H), 8.91 (s, 1H), 8.30 (s, 1H), 7.92-7.84 (m, 2H), 7.67-7.63 (m, 1H), 7.10-7.03 (m, 1H), 6.88-6.85 (m, 1H), 6.65-6.61 (m, 1H), 3.73 (s, 3H), 2.83 (s, 3H), 2.32-2.27 (m, 2H), 1.87-1.86 (m, 1H), 1.57-1.55 (m, 1H).

### Example 141: trans-6-(5-(2-(4-Fluoro-3-methoxyphenyl)cyclopropyl)-[2,2′-bipyrimidin]-4-yl)-2-methylbenzo[d]thiazole (single enantiomer II)

m/z: 470 [M+H]⁺ observed. ¹H NMR (400 MHz, DMSO-d₆): δ 9.01 (d, 2H), 8.91 (s, 1H), 8.30 (s, 1H), 7.92-7.84 (m, 2H), 7.67-7.63 (m, 1H), 7.10-7.03 (m, 1H), 6.88-6.85 (m, 1H), 6.65-6.61 (m, 1H), 3.73 (s, 3H), 2.83 (s, 3H), 2.32-2.27 (m, 2H), 1.87-1.86 (m, 1H), 1.57-1.55 (m, 1H).

The following examples were prepared in a similar manner as trans-6-(5-(2-(4-fluoro-3-methoxyphenyl)cyclopropyl)-[2,2′-bipyrimidin]-4-yl)-2-methylbenzo[d]thiazole from an appropriately substituted (E)-2,4-dichloro-5-(styryl)pyrimidine and an appropriately substituted aryl boronic acid or aryl boronic ester:

### Example 142: trans-5-(2-(4-Fluoro-3-methoxyphenyl)cyclopropyl)-4-phenyl-2,2′-bipyrimidine (single enantiomer I)

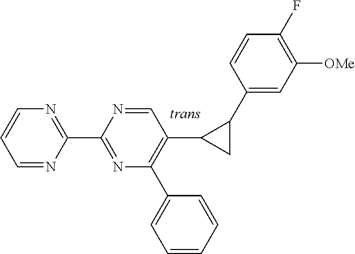 [SMILES: COc1cc(C2CC2c2cnc(-c3ncccn3)nc2-c2ccccc2)ccc1F]

### Example 143: trans-5-(2-(4-Fluoro-3-methoxyphenyl)cyclopropyl)-4-phenyl-2,2′-bipyrimidine (single enantiomer II)

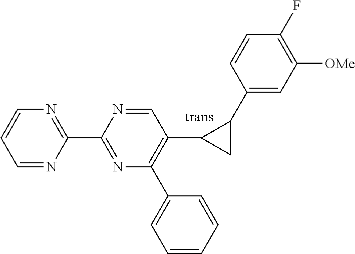 [SMILES: COc1cc(C2CC2c2cnc(-c3ncccn3)nc2-c2ccccc2)ccc1F]

A mixture of enantiomers (65 mg) was separated by chiral HPLC on a CHIRALCEL® OJ-H column using n-hexane and EtOH (65:35) to give trans-5-(2-(4-fluoro-3-methoxyphenyl)cyclopropyl)-4-phenyl-2,2′-bipyrimidine (single enantiomer I) as an brown solid (faster eluting enantiomer, 27 mg, 41%, m/z: 399 [M+H]⁺ observed), and trans-5-(2-(4-fluoro-3-methoxyphenyl)cyclopropyl)-4-phenyl-2,2′-bipyrimidine (single enantiomer II) as an brown solid (slower eluting enantiomer, 26 mg, 40%, m/z: 399 [M+H]⁺ observed).

### Example 142: trans-5-(2-(4-Fluoro-3-methoxyphenyl)cyclopropyl)-4-phenyl-2,2′-bipyrimidine (single enantiomer I)

m/z: 399 [M+H]⁺ observed. ¹H NMR (300 MHz, DMSO-d₆): δ 9.00 (d, 2H), 8.86 (s, 1H), 7.71-7.68 (m, 2H), 7.66-7.62 (m, 1H), 7.46-7.43 (m, 1H), 7.39-7.34 (m, 2H), 7.11-7.05 (m, 1H), 6.92-6.88 (m, 1H), 6.65 (s, 1H), 3.79 (s, 3H), 2.41-2.37 (m, 1H), 2.27-2.20 (m, 1H), 1.81-1.74 (m, 1H), 1.59-1.54 (m, 1H).

### Example 143: trans-5-(2-(4-Fluoro-3-methoxyphenyl)cyclopropyl)-4-phenyl-2,2′-bipyrimidine (single enantiomer II)

m/z: 399 [M+H]⁺ observed. ¹H NMR (300 MHz, DMSO-d₆): δ 9.00 (d, 2H), 8.86 (s, 1H), 7.71-7.68 (m, 2H), 7.66-7.62 (m, 1H), 7.46-7.43 (m, 1H), 7.39-7.34 (m, 2H), 7.11-7.05 (m, 1H), 6.92-6.88 (m, 1H), 6.65 (s, 1H), 3.79 (s, 3H), 2.41-2.37 (m, 1H), 2.27-2.20 (m, 1H), 1.81-1.74 (m, 1H), 1.59-1.54 (m, 1H).

### Example 144: trans-5-(2-(4-Fluoro-3-methoxyphenyl)cyclopropyl)-4-(piperidin-1-yl)-2,2′-bipyrimidine (single enantiomer I)

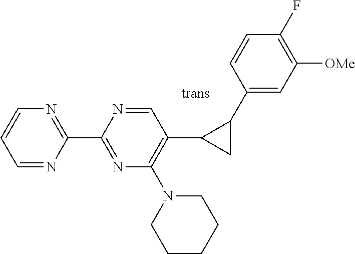 [SMILES: COc1cc(C2CC2c2cnc(-c3ncccn3)nc2N2CCCCC2)ccc1F]

### Example 145: trans-5-(2-(4-Fluoro-3-methoxyphenyl)cyclopropyl)-4-(piperidin-1-yl)-2,2′-bipyrimidine (single enantiomer II)

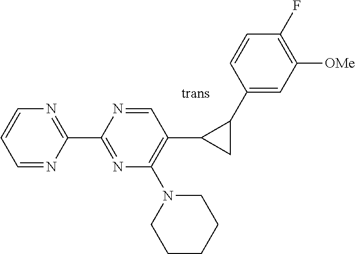 [SMILES: COc1cc(C2CC2c2cnc(-c3ncccn3)nc2N2CCCCC2)ccc1F]

### (E)-2,4-Dichloro-5-(4-fluoro-3-methoxystyryl)pyrimidine

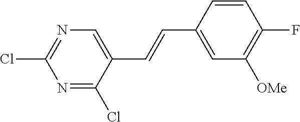 [SMILES: COc1cc(/C=C/c2cnc(Cl)nc2Cl)ccc1F]

To a solution of (E)-2-(4-fluoro-3-methoxystyryl)-4,4,5,5-tetramethyl-1,3,2-dioxaborolane (2.5 g, 9.0 mmol) in1,4-dioxane/water(1:1, 25 mL) was added 2,4-dichloro-5-iodopyrimidine (3.7 g, 13.5 mmol) and K₂CO₃ (2.4 g, 17.3 mmol) at rt, and the mixture was purged with N₂ gas for 10 min. Tetrakis(triphenylphosphine)palladium(0) (520 mg, 0.45 mmol) was added, and the mixture was degassed with N₂ gas for 10 min. The reaction mixture was heated at 90° C. for 16 h in a sealed tube. The mixture was cooled to rt, diluted with water (200 mL), and extracted with EtOAc (2×200 mL). The combined organic phase was washed with saturated aqueous brine solution (200 mL), dried over anhydrous sulfate, filtered, and evaporated under reduced pressure. The residue was purified using normal phase SiO₂ chromatography (0-30% EtOAc/petroleum ether) to give (E)-2,4-dichloro-5-(4-fluoro-3-methoxystyryl)pyrimidine as a yellow solid (0.90 g, 33% yield, m/z: 299 [M+H]⁺ observed). ¹H NMR (400 MHz, CDCl₃): δ 8.79 (d, 1H), 7.16-7.03 (m, 5H), 3.96 (s, 3H).

### trans-2,4-Dichloro-5-(2-(4-fluoro-3-methoxyphenyl)cyclopropyl)pyrimidine

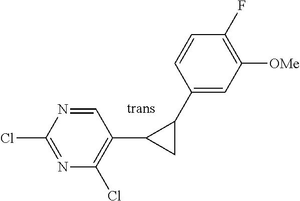 [SMILES: COc1cc(C2CC2c2cnc(Cl)nc2Cl)ccc1F]

To a solution of (E)-2-chloro-5-(4-fluoro-3-methoxystyryl)-4-methoxypyrimidine (0.90 g, 3.0 mmol) in THF (5 mL) at 0° C. was added Pd₃(OAc)₆ (200 mg, 0.30 mmol) and freshly prepared ethereal diazomethane [prepared from of N-methyl-N-nitroso urea (6.20 g, 60.3 mmol), KOH solution (50% aqueous, 50 mL) and Et₂O (50 mL) at 0° C.] and stirred at 0-5° C. for 16 h. The reaction mixture was filtered through CELITE® pad and the filtrate was concentrated under reduced pressure. The residue was dissolved in THF (5 mL) at 0° C. and Pd₃(OAc)₆ (200 mg, 0.30 mmol) was added, followed by freshly prepared ethereal diazomethane [prepared from of N-methyl-N-nitroso urea (6.20 g, 60.3 mmol), KOH solution (50% aqueous, 50 mL) and Et₂O (50 mL) at 0° C.] and stirred at 0-5° C. for 16 h. The reaction mixture was filtered through CELITE® pad and the filtrate was concentrated under reduced pressure. The residue was purified by normal phase SiO₂ chromatography (0-10% EtOAc/petroleum ether) to give trans-2,4-dichloro-5-(2-(4-fluoro-3-methoxyphenyl)cyclopropyl)pyrimidine as a yellow oil (0.40 g, 42% yield, m/z: 313 [M+H]⁺ observed).

### trans-2-Chloro-5-(2-(4-fluoro-3-methoxyphenyl)cyclopropyl)-4-(piperidin-1-yl)pyrimidine

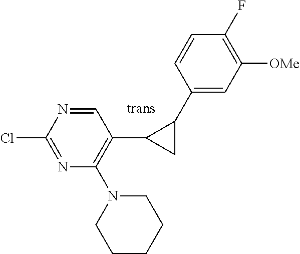 [SMILES: COc1cc(C2CC2c2cnc(Cl)nc2N2CCCCC2)ccc1F]

To a solution of trans-2,4-dichloro-5-(2-(4-fluoro-3-methoxyphenyl)cyclopropyl)pyrimidine (0.40 g, 1.3 mmol) in THF (4 mL) was added DIPEA (0.60 mL, 3.4 mmol) and piperidine (0.10 mL, 1.4 mmol) at rt and stirred for 3 h. The reaction mixture was diluted with water (100 mL) and extracted with EtOAc (2×100 mL). The combined organic phase was washed with saturated aqueous brine solution (100 mL), dried over anhydrous sodium sulfate, filtered and evaporated under reduced pressure. The residue was purified by normal phase SiO₂ chromatography (0-20% EtOAc/petroleum ether) to give trans-2-chloro-5-(2-(4-fluoro-3-methoxyphenyl)cyclopropyl)-4-(piperidin-1-yl)pyrimidine as an orange gum (0.36 g, 77% yield, m/z: 362 [M+H]⁺ observed). ¹H NMR (400 MHz, CDCl₃): δ 7.94 (s, 1H), 7.03-6.98 (m, 1H), 6.73-6.70 (m, 1H), 6.63-6.59 (m, 1H), 3.89 (s, 3H), 3.57-3.54 (m, 4H), 2.05-1.94 (m, 2H), 1.60-1.56 (m, 2H), 1.53-1.48 (m, 6H).

### trans-5-(2-(4-Fluoro-3-methoxyphenyl)cyclopropyl)-4-(piperidin-1-yl)-2,2′-bipyrimidine

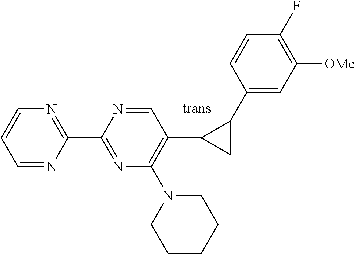 [SMILES: COc1cc(C2CC2c2cnc(-c3ncccn3)nc2N2CCCCC2)ccc1F]

To a solution of trans-2-chloro-5-(2-(4-fluoro-3-methoxyphenyl)cyclopropyl)-4-(piperidin-1-yl)pyrimidine (0.27 g, 0.75 mmol) in DMF (5 mL) was added 2-(tributylstannyl)pyrimidine (0.24 mL, 0.76 mmol), tetraethylammonium chloride (0.13 g, 0.78 mmol), and K₂CO₃ (0.21 g, 1.5 mmol) at rt and the mixture was purged with N₂ gas for 10 min. Bis(triphenylphosphine)palladium(II) dichloride (52 mg, 0.074 mmol) was added, and the reaction mixture was purged with N₂ gas for 10 min. The reaction mixture was stirred at 110° C. for 16 h. The reaction mixture was cooled to rt, diluted with water (100 mL), and extracted with EtOAc (2×100 mL). The combined organic phase was washed with saturated aqueous brine solution (100 mL), dried over anhydrous sodium sulfate, filtered, and evaporated to under reduced pressure. The residue was purified by reverse phase HPLC to give trans-5-(2-(4-fluoro-3-methoxyphenyl)cyclopropyl)-4-(piperidin-1-yl)-2,2′-bipyrimidine as an off-white solid (150 mg, 49% yield, m/z: 406 [M+H]⁺ observed). ¹H NMR (400 MHz, DMSO-d₆): δ 8.94-8.93 (m, 2H), 8.31 (s, 1H), 7.58-7.56 (m, 1H), 7.14-7.09 (m, 1H), 7.04-7.01 (m, 1H), 6.81-6.78 (m, 1H), 3.85 (s, 3H), 3.53-3.45 (m, 4H), 2.27-2.22 (m, 1H), 2.16-2.11 (m, 1H), 1.77-1.72 (m, 1H), 1.54-1.47 (m, 5H), 1.37-1.34 (m, 2H).

A mixture of enantiomers (150 mg) was separated by chiral HPLC on a CHIRALCEL® IG column using n-hexane and EtOH (30:70) to give trans-5-(2-(4-fluoro-3-methoxyphenyl)cyclopropyl)-4-(piperidin-1-yl)-2,2′-bipyrimidine (single enantiomer I) as an off-white solid (faster eluting enantiomer, 35 mg, 23%, m/z: 406 [M+H]+ observed), and trans-5-(2-(4-fluoro-3-methoxyphenyl)cyclopropyl)-4-(piperidin-1-yl)-2,2′-bipyrimidine (single enantiomer II) as an off-white solid (slower eluting enantiomer, 34 mg, 22%, m/z: 406 [M+H]⁺ observed).

### Example 144: trans-5-(2-(4-fluoro-3-methoxyphenyl)cyclopropyl)-4-(piperidin-1-yl)-2,2′-bipyrimidine (single enantiomer I)

m/z: 406 [M+H]⁺ observed. ¹H NMR (400 MHz, DMSO-d₆): δ 8.94-8.93 (m, 2H), 8.31 (s, 1H), 7.58-7.56 (m, 1H), 7.14-7.09 (m, 1H), 7.04-7.01 (m, 1H), 6.81-6.78 (m, 1H), 3.85 (s, 3H), 3.53-3.45 (m, 4H), 2.27-2.22 (m, 1H), 2.16-2.11 (m, 1H), 1.77-1.72 (m, 1H), 1.54-1.47 (m, 5H), 1.37-1.34 (m, 2H).

### Example 145: trans-5-(2-(4-fluoro-3-methoxyphenyl)cyclopropyl)-4-(piperidin-1-yl)-2,2′-bipyrimidine (single enantiomer II)

m/z: 406 [M+H]⁺ observed. ¹H NMR (400 MHz, DMSO-d₆): δ 8.94-8.93 (m, 2H), 8.31 (s, 1H), 7.58-7.56 (m, 1H), 7.14-7.09 (m, 1H), 7.04-7.01 (m, 1H), 6.81-6.78 (m, 1H), 3.85 (s, 3H), 3.53-3.45 (m, 4H), 2.27-2.22 (m, 1H), 2.16-2.11 (m, 1H), 1.77-1.72 (m, 1H), 1.54-1.47 (m, 5H), 1.37-1.34 (m, 2H).

### Example 146: trans-5-(2-(4-Fluoro-3,5-dimethoxyphenyl)cyclopropyl)-2,2′-bipyrimidine (single enantiomer I)

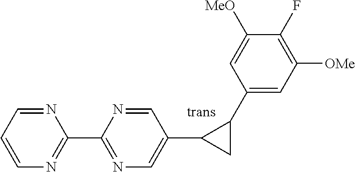 [SMILES: COc1cc(C2CC2c2cnc(-c3ncccn3)nc2)cc(OC)c1F]

### Example 147: trans-5-(2-(4-Fluoro-3,5-dimethoxyphenyl)cyclopropyl)-2,2′-bipyrimidine (single enantiomer II)

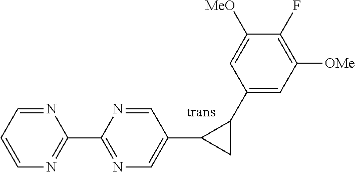 [SMILES: COc1cc(C2CC2c2cnc(-c3ncccn3)nc2)cc(OC)c1F]

### 2-Chloro-5-((trimethylsilyl)ethynyl)pyrimidine

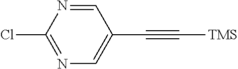 [SMILES: C[Si](C)(C)C#Cc1cnc(Cl)nc1]

To a mixture of 2-chloro-5-iodo-pyrimidine (25 g, 104 mmol), ethynyl(trimethyl)silane (20 mL, 141 mmol) and triethylamine (27 mL, 197 mmol) in THF (500 mL) was added copper(I) iodide (0.59 g, 3 mmol) and bis(triphenylphosphine)palladium(II) dichloride (2 g, 3 mmol) in one portion under N₂. The mixture was stirred at 50° C. for 16 hours. To the mixture was added H₂O (300 mL) and extracted with EtOAc (2×100 mL). The combined organic phase was washed with saturated aqueous brine solution (80 mL), dried over Na₂SO₄, filtered and concentrated in vacuum. The residue was purified by normal phase SiO₂ chromatography (0-5% EtOAc/petroleum ether) to give 2-chloro-5-((trimethylsilyl)ethynyl)pyrimidine as a yellow solid (16 g, 73% yield, m/z: 211 [M+H]⁺ observed). ¹H NMR (400 MHz, CDCl₃): δ 8.38 (s, 2H), 0.00 (s, 9 H).

### 2-Chloro-5-ethynylpyrimidine

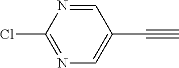

To a mixture of 2-chloro-5-((trimethylsilyl)ethynyl)pyrimidine (16 g, 76 mmol) in ACN (120 mL) and H₂O (40 mL) was added KOH (8.5 g, 152 mmol) in one portion under N₂. The mixture was stirred at rt for 1 hour. To the mixture was added H₂O (80 mL) and extracted with EtOAc (3×40 mL). The combined organic phase was washed with saturated aqueous brine solution (50 mL), dried over Na₂SO₄, filtered and concentrated in vacuum. The residue was purified by normal phase SiO₂ chromatography (0-6% EtOAc/petroleum ether) to give 2-chloro-5-ethynylpyrimidine as a white solid (3 g, 29% yield, m/z: 139 [M+H]⁺ observed). ¹H NMR (400 MHz, CDCl₃): δ 8.71 (s, 2H), 3.46 (s, 1 H).

### (E)-2-Chloro-5-(2-(4,4,5,5-tetramethyl-1,3,2-dioxaborolan-2-yl)vinyl)pyrimidine

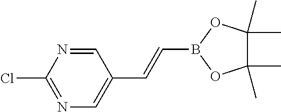 [SMILES: CC1(C)OB(/C=C/c2cnc(Cl)nc2)OC1(C)C]

To a mixture of 2-chloro-5-ethynylpyrimidine (3 g, 22 mmol) in CH₂Cl₂ (30 mL) was added di(cyclopentadienyl)zirconium(IV) chloride hydride (1.2 g, 4 mmol) in one portion under N₂. The mixture was stirred at rt for 1.5 hours. Then to the mixture was added 4,4,5,5-tetramethyl-1,3,2-dioxaborolane (2.8 g, 21.7 mmol) in one portion under N₂. The mixture was stirred at 60° C. for 16 hours. The mixture was purified without workup. The residue was purified by normal phase SiO₂ chromatography (0-9% EtOAc/petroleum ether) to give (E)-2-chloro-5-(2-(4,4,5,5-tetramethyl-1,3,2-dioxaborolan-2-yl)vinyl)pyrimidine as a white solid (3 g, 52% yield). ¹H NMR (400 MHz, CDCl₃): δ 8.69 (s, 2H), 7.27 (d, J=18.8 Hz, 1H), 6.33 (s, d, J=18.8 Hz, 1H), 1.32 (s, 12H).

### Diazomethane:

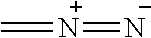

A two-necked round-bottomed flask, equipped with a dropping funnel and distillation apparatus was cooled in acetone-dry ice bath. A mixture of KOH (14 g, 252 mmol) in H₂O (20 mL) and 2-ethoxyethanol (60 mL) was heated to 70° C. and a solution of N,4-dimethyl-N-nitroso-benzenesulfonamide (40 g, 187 mmol) in ethoxyethane (300 mL) was added dropwise over 1 h. The ethereal diazomethane solution was collected at −20° C. to give diazomethane (280 mL, 0.75 M in Et₂O) as a yellow solution, which was used in the next step without further purification.

### trans-2-Chloro-5-(2-(4,4,5,5-tetramethyl-1,3,2-dioxaborolan-2-yl)cyclopropyl)pyrimidine

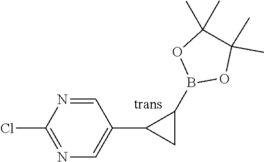 [SMILES: CC1(C)OB(C2CC2c2cnc(Cl)nc2)OC1(C)C]

To a mixture of (E)-2-chloro-5-(2-(4,4,5,5-tetramethyl-1,3,2-dioxaborolan-2-yl)vinyl)pyrimidine (3 g, 11.3 mmol) in THF (30 mL) was added diazomethane (0.75 M in Et₂O, 225 mL) in ethoxyethane in one portion at −30° C. under N₂. The mixture was stirred at −30° C. for 10 min, then to the mixture was added Pd(OAc)₂ (0.5 g, 2.3 mmol) in one portion under N₂. The mixture was stirred at −30° C. for 20 min. The mixture was filtered through CELITE® pad and washed with EtOAc (20 mL). The filtrate was concentrated in vacuum to give trans-2-chloro-5-(2-(4,4,5,5-tetramethyl-1,3,2-dioxaborolan-2-yl)cyclopropyl)pyrimidine as a yellow oil (3.2 g, 65% yield, m/z: 281 [M+H]⁺ observed), which was used in the next step without further purification.

### trans-2-Chloro-5-(2-(4-fluoro-3,5-dimethoxyphenyl)cyclopropyl)pyrimidine

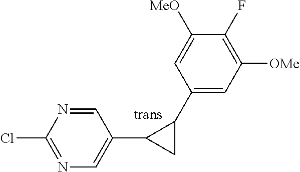 [SMILES: COc1cc(C2CC2c2cnc(Cl)nc2)cc(OC)c1F]

To a mixture of 5-bromo-2-fluoro-1,3-dimethoxybenzene (0.8 g, 3.4 mmol), crude 2-chloro-5-[2-(4,4,5,5-tetramethyl-1,3,2-dioxaborolan-2-yl)cyclopropyl]pyrimidine (1.1 g, 3.7 mmol) and Cs₂CO₃ (2.2 g, 6.8 mmol) in THF-E120 (4:1, 10 mL) was added Pd(dppf)Cl₂. CH₂Cl₂ (0.27 mg, 0.34 mmol) in one portion under N₂. The mixture was stirred at 80° C. for 16 hours. To the mixture was added H₂O (40 mL) and extracted with EtOAc (3×20 mL). The combined organic phase was washed with saturated aqueous brine solution (30 mL), dried over Na₂SO₄, filtered and concentrated in vacuum. The residue was purified by normal phase SiO₂ chromatography (0-25% EtOAc/petroleum ether) to give trans-2-chloro-5-(2-(4-fluoro-3,5-dimethoxyphenyl)cyclopropyl)pyrimidine as a yellow solid (0.36 g, 34% yield, m/z: 309 [M+H]⁺ observed). ¹H NMR (400 MHz, CDCl₃): δ 8.44 (s, 2H), 6.39 (d, J=7.2 Hz, 2H), 3.90 (s, 6H), 2.24-2.22 (m, 1H), 2.10-2.08 (m, 1H), 1.53-1.49 (m, 1H), 0.99-0.93 (m, 1H). trans-5-(2-(4-Fluoro-3,5-dimethoxyphenyl)cyclopropyl)-2,2′-bipyrimidine:

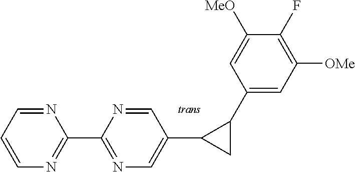 [SMILES: COc1cc(C2CC2c2cnc(-c3ncccn3)nc2)cc(OC)c1F]

To a mixture of trans-2-chloro-5-(2-(4-fluoro-3,5-dimethoxyphenyl)cyclopropyl)pyrimidine (310 mg, 1 mmol), 2-(tributylstannyl)pyrimidine (389 mg, 1.1 mmol), K₂CO₃ (153 mg, 1.1 mmol) and tetraethylammonium chloride (166 mg, 1 mmol) in DMF (4 mL) was added Pd(dppf)Cl₂ (73.5 mg, 0.1 mmol) in one portion under N₂. The mixture was stirred at 110° C. for 16 hours. The mixture was purified without workup. The residue was purified by normal phase SiO₂ chromatography (0-9% MeOH/CH₂Cl₂) followed by reverse phase HPLC to give trans-5-(2-(4-fluoro-3,5-dimethoxyphenyl)cyclopropyl)-2,2′-bipyrimidine as a white solid (130 mg, 36% yield, m/z: 353 [M+H]⁺ observed). ¹H NMR (400 MHz, DMSO-d₆): δ 8.99 (d, J=5.2 Hz, 2H), 8.85 (s, 2H), 7.62 (t, J=4.8 Hz, 1H), 6.64 (d, J=7.2 Hz, 2H), 3.83 (s, 6H), 2.50-2.45 (m, 1H), 2.41-2.36 (m, 1H), 1.71(t, J=7.6 Hz, 2H). A mixture of enantiomers trans-5-(2-(4-fluoro-3,5-dimethoxyphenyl)cyclopropyl)-2,2′-bipyrimidine (130 mg) was separated by SFC (supercritical fluid chromatography) on a CHIRALPAK® AD column using liquid CO₂ and IPA [0.1% aqueous NH₃ modifier] (50:50) to give trans-5-(2-(4-fluoro-3,5-dimethoxyphenyl)cyclopropyl)-2,2′-bipyrimidine (single enantiomer I) as a white solid (faster eluting enantiomer, 46 mg, 35% yield, m/z: 353 [M+H]⁺ observed) and trans-5-(2-(4-fluoro-3,5-dimethoxyphenyl)cyclopropyl)-2,2′-bipyrimidine (single enantiomer II) (slower eluting enantiomer, 47 mg, 36% yield, m/z: 353 [M+H]⁺ observed).

### Example 146: trans-5-(2-(4-Fluoro-3,5-dimethoxyphenyl)cyclopropyl)-2,2′-bipyrimidine (single enantiomer I)

m/z: 353 [M+H]⁺ observed. ¹H NMR (400 MHz, DMSO-d₆): δ 8.99 (d, J=5.2 Hz, 2H), 8.85 (s, 2H), 7.62 (t, J=4.8 Hz, 1H), 6.64 (d, J=7.2 Hz, 2H), 3.83 (s, 6H), 2.50-2.45 (m, 1H), 2.41-2.36 (m, 1H), 1.71(t, J=7.6 Hz, 2H).

### Example 147: trans-5-(2-(4-Fluoro-3,5-dimethoxyphenyl)cyclopropyl)-2,2′-bipyrimidine (single enantiomer II)

m/z: 353 [M+H]⁺ observed. ¹H NMR (400 MHz, DMSO-d₆): δ 8.99 (d, J=5.2 Hz, 2H), 8.85 (s, 2H), 7.62 (t, J=4.8 Hz, 1H), 6.64 (d, J=7.2 Hz, 2H), 3.83 (s, 6H), 2.50-2.45 (m, 1H), 2.41-2.36 (m, 1H), 1.71(t, J=7.6 Hz, 2H).

The following examples were prepared in a similar manner as trans-5-(2-(4-fluoro-3,5-dimethoxyphenyl)cyclopropyl)-2,2′-bipyrimidine from trans-2-chloro-5-(2-(4,4,5,5-tetramethyl-1,3,2-dioxaborolan-2-yl)cyclopropyl)pyrimidine and an appropriately substituted aryl bromide:

### Example 148: trans-5-(2-(3-Chloro-4-fluoro-5-methoxyphenyl)cyclopropyl)-2,2′-bipyrimidine (single enantiomer I)

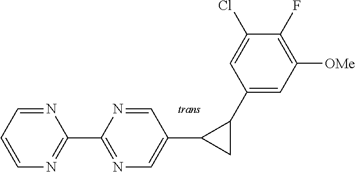 [SMILES: COc1cc(C2CC2c2cnc(-c3ncccn3)nc2)cc(Cl)c1F]

### Example 149: trans-5-(2-(3-Chloro-4-fluoro-5-methoxyphenyl)cyclopropyl)-2,2′-bipyrimidine (single enantiomer II)

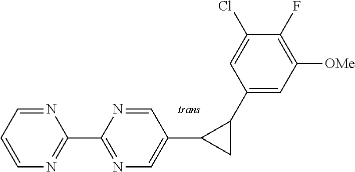 [SMILES: COc1cc(C2CC2c2cnc(-c3ncccn3)nc2)cc(Cl)c1F]

A mixture of enantiomers of trans-5-(2-(3-chloro-4-fluoro-5-methoxyphenyl)cyclopropyl)-2,2′-bipyrimidine (80 mg) was separated by SFC (supercritical fluid chromatography) on a CHIRALCEL® OD column using liquid CO₂ and MeOH [0.1% aqueous NH₃ modifier] (40:60) to give trans-5-(2-(3-chloro-4-fluoro-5-methoxyphenyl)cyclopropyl)-2,2′-bipyrimidine (single enantiomer I) as a white solid (faster eluting enantiomer, 23 mg, 27% yield, m/z: 357 [M+H]⁺ observed) and trans-5-(2-(3-chloro-4-fluoro-5-methoxyphenyl)cyclopropyl)-2,2′-bipyrimidine (single enantiomer II) (slower eluting enantiomer, 24 mg, 30% yield, m/z: 357 [M+H]⁺ observed).

### Example 148: trans-5-(2-(3-Chloro-4-fluoro-5-methoxyphenyl)cyclopropyl)-2,2′-bipyrimidine (single enantiomer I)

m/z: 357 [M+H]⁺ observed. ¹H NMR (400 MHz, DMSO-d₆): δ 8.99 (d, J=5.2 Hz, 2H), 8.85 (s, 2H), 7.62 (t, J=4.8 Hz, 1H), 7.05 (dd, J=2 Hz, J=7.6 Hz, 1H), 6.99 (dd, J=2 Hz, J=6 Hz, 1H), 3.88 (s, 3H), 2.56-2.55 (m, 1H), 2.45-2.40 (m, 1H), 1.77-1.69 (m, 2H).

### Example 149: trans-5-(2-(3-Chloro-4-fluoro-5-methoxyphenyl)cyclopropyl)-2,2′-bipyrimidine (single enantiomer II)

m/z: 357 [M+H]⁺ observed. ¹H NMR (400 MHz, DMSO-d₆): δ 8.99 (d, J=5.2 Hz, 2H), 8.85 (s, 2H), 7.62 (t, J=4.8 Hz, 1H), 7.05 (dd, J=2 Hz, J=7.6 Hz, 1H), 6.99 (dd, J=2 Hz, J=6 Hz, 1H), 3.88 (s, 3H), 2.56-2.55 (m, 1H), 2.45-2.40 (m, 1H), 1.77-1.69 (m, 2H).

### Example 150: trans-5-(2-(4-Fluoro-3-methoxy-5-methylphenyl)cyclopropyl)-2,2′-bipyrimidine (single enantiomer I)

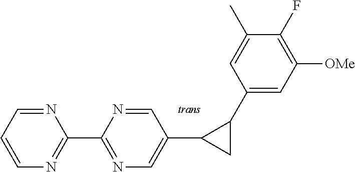 [SMILES: COc1cc(C2CC2c2cnc(-c3ncccn3)nc2)cc(C)c1F]

### Example 151: trans-5-(2-(4-Fluoro-3-methoxy-5-methylphenyl)cyclopropyl)-2,2′-bipyrimidine (single enantiomer II)

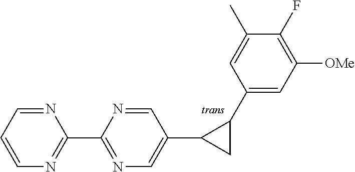 [SMILES: COc1cc(C2CC2c2cnc(-c3ncccn3)nc2)cc(C)c1F]

A mixture of enantiomers of trans-5-(2-(4-fluoro-3-methoxy-5-methylphenyl)cyclopropyl)-2,2′-bipyrimidine (70 mg) was separated by SFC (supercritical fluid chromatography) on a CHIRALCEL® OD column using liquid CO₂ and MeOH [0.1% aqueous NH₃ modifier] (40:60) to give trans-5-(2-(4-fluoro-3-methoxy-5-methylphenyl)cyclopropyl)-2,2′-bipyrimidine (single enantiomer I) as a white solid (faster eluting enantiomer, 31 mg, 43% yield, m/z: 337 [M+H]⁺ observed) and trans-5-(2-(4-fluoro-3-methoxy-5-methylphenyl)cyclopropyl)-2,2′-bipyrimidine (single enantiomer II) (slower eluting enantiomer, 27 mg, 37% yield, m/z: 337 [M+H]⁺ observed).

### Example 150: trans-5-(2-(4-Fluoro-3-methoxy-5-methylphenyl)cyclopropyl)-2,2′-bipyrimidine (single enantiomer I)

m/z: 337 [M+H]⁺ observed. ¹H NMR (400 MHz, DMSO-d₆): δ 8.99 (d, J=4.8 Hz, 2H), 8.84 (s, 2H), 7.62 (t, J=4.8 Hz, 1H), 6.85 (dd, J=1.6 Hz, J=7.6 Hz, 1H), 6.66 (dd, J=1.2 Hz, J=6.4 Hz, 1H), 3.82 (s, 3H), 2.47-2.45 (m, 1H), 2.34-2.32 (m, 1H), 2.19 (d, J=2 Hz, 3H), 1.71-1.63 (m, 2H).

### Example 151: trans-5-(2-(4-Fluoro-3-methoxy-5-methylphenyl)cyclopropyl)-2,2′-bipyrimidine (single enantiomer II):

m/z: 337 [M+H]⁺ observed. ¹H NMR (400 MHz, DMSO-d₆): δ 8.99 (d, J=4.8 Hz, 2H), 8.84 (s, 2H), 7.62 (t, J=4.8 Hz, 1H), 6.85 (dd, J=1.6 Hz, J=7.6 Hz, 1H), 6.66 (dd, J=1.2 Hz, J=6.4 Hz, 1H), 3.82 (s, 3H), 2.47-2.45 (m, 1H), 2.34-2.32 (m, 1H), 2.19 (d, J=2 Hz, 3H), 1.71-1.63 (m, 2H).

### Example 152: trans-5-(2-(3-Methoxy-4-(trifluoromethoxy)phenyl)cyclopropyl)-2,2′-bipyrimidine

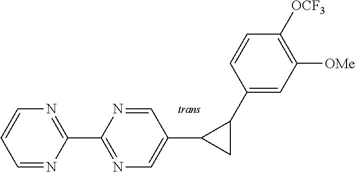 [SMILES: COc1cc(C2CC2c2cnc(-c3ncccn3)nc2)ccc1OC(F)(F)F]

m/z: 389 [M+H]⁺ observed. ¹H NMR (400 MHz, CDCl₃) δ 9.02 (d, J=4.9 Hz, 2H), 8.79 (s, 2H), 7.43 (t, J=4.8 Hz, 1H), 7.19 (dd, J=8.3, 1.4 Hz, 1H), 6.81 (d, J=2.1 Hz, 1H), 6.72 (dd, J=8.3, 2.1 Hz, 1H), 3.90 (s, 3H), 2.41-2.31 (m, 1H), 2.30-2.20 (m, 1H), 1.71-1.62 (m, 2H).

### Example 153: trans-5-(2-(3-Methoxy-4-(trifluoromethyl)phenyl)cyclopropyl)-2,2′-bipyrimidine

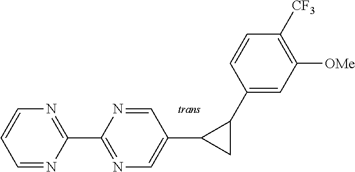 [SMILES: COc1cc(C2CC2c2cnc(-c3ncccn3)nc2)ccc1C(F)(F)F]

m/z: 373 [M+H]⁺ observed. ¹H NMR (400 MHz, CDCl₃) δ 9.02 (d, J=4.8 Hz, 2H), 8.79 (s, 2H), 7.51 (d, J=8.0 Hz, 1H), 7.43 (t, J=4.8 Hz, 1H), 6.82 (s, 1H), 6.77 (d, J=8.0 Hz, 1H), 3.93 (s, 3H), 2.39 (td, J=7.4, 7.0, 4.5 Hz, 1H), 2.30 (td, J=7.8, 4.6 Hz, 1H), 1.76-1.67 (m, 2H).

### Example 154: trans-5-(2-(5-Chloro-4-fluoro-2-(3-methoxypropoxy)phenyl)cyclopropyl)-2,2′-bipyrimidine (single enantiomer I)

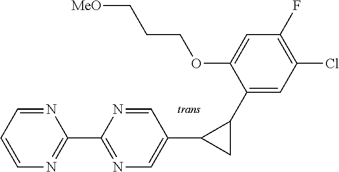 [SMILES: COCCCOc1cc(F)c(Cl)cc1C1CC1c1cnc(-c2ncccn2)nc1]

### Example 155: trans-5-(2-(5-Chloro-4-fluoro-2-(3-methoxypropoxy)phenyl)cyclopropyl)-2,2′-bipyrimidine (single enantiomer II)

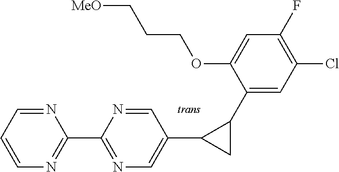 [SMILES: COCCCOc1cc(F)c(Cl)cc1C1CC1c1cnc(-c2ncccn2)nc1]

A mixture of enantiomers of trans-5-(2-(5-chloro-4-fluoro-2-(3-methoxypropoxy)phenyl)cyclopropyl)-2,2′-bipyrimidine (100 mg) was separated by SFC (supercritical fluid chromatography) on a CHIRALCEL® OD column using liquid CO₂ and EtOH [0.1% aqueous NH₃ modifier] (55:45) to give trans-5-(2-(5-chloro-4-fluoro-2-(3-methoxypropoxy)phenyl)cyclopropyl)-2,2′-bipyrimidine (single enantiomer I) as a white solid (faster eluting enantiomer, 26 mg, 25% yield, m/z: 415 [M+H]⁺ observed) and trans-5-(2-(5-chloro-4-fluoro-2-(3-methoxypropoxy)phenyl)cyclopropyl)-2,2′-bipyrimidine (single enantiomer II) as a white solid (slower eluting enantiomer, 23 mg, 23% yield, m/z: 415 [M+H]⁺ observed).

### Example 154: trans-5-(2-(5-Chloro-4-fluoro-2-(3-methoxypropoxy)phenyl)cyclopropyl)-2,2′-bipyrimidine (single enantiomer I)

m/z: 415 [M+H]⁺ observed. ¹H NMR (400 MHz, DMSO-d₆): δ 8.99 (d, J=4.8 Hz, 2H), 8.87 (s, 2H), 7.62 (t, J=4.8 Hz, 1H), 7.29 (d, J=8.8 Hz, 1H), 7.12 (d, J=11.6 Hz, 1H), 4.03 (t, J=6 Hz, 2H), 3.25 (t, J=6.4 Hz, 2H), 3.06 (s, 3H), 2.46-2.45 (m, 1H), 2.22-2.18 (m, 1H), 1.86-1.83 (m, 2H), 1.77-1.75 (m, 1H), 1.66-1.64 (m, 1H).

### Example 155: trans-5-(2-(5-Chloro-4-fluoro-2-(3-methoxypropoxy)phenyl)cyclopropyl)-2,2′-bipyrimidine (single enantiomer II)

m/z: 415 [M+H]⁺ observed. ¹H NMR (400 MHz, DMSO-d₆): δ 8.99 (d, J=4.8 Hz, 2H), 8.87 (s, 2H), 7.62 (t, J=4.8 Hz, 1H), 7.29 (d, J=8.8 Hz, 1H), 7.12 (d, J=11.6 Hz, 1H), 4.03 (t, J=6 Hz, 2H), 3.25 (t, J=6.4 Hz, 2H), 3.06 (s, 3H), 2.46-2.45 (m, 1H), 2.22-2.18 (m, 1H), 1.86-1.83 (m, 2H), 1.77-1.75 (m, 1H), 1.66-1.64 (m, 1H).

### Example 156: trans-5-(2-(5-Chloro-4-fluoro-2-(2-methoxyethoxy)phenyl)cyclopropyl)-2,2′-bipyrimidine (single enantiomer I)

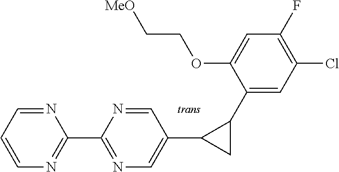 [SMILES: COCCOc1cc(F)c(Cl)cc1C1CC1c1cnc(-c2ncccn2)nc1]

### Example 157: trans-5-(2-(5-Chloro-4-fluoro-2-(2-methoxyethoxy)phenyl)cyclopropyl)-2,2′-bipyrimidine (single enantiomer II)

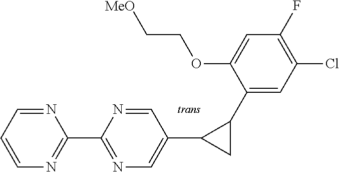 [SMILES: COCCOc1cc(F)c(Cl)cc1C1CC1c1cnc(-c2ncccn2)nc1]

A mixture of enantiomers of trans-5-(2-(5-chloro-4-fluoro-2-(2-methoxyethoxy)phenyl)cyclopropyl)-2,2′-bipyrimidine (90 mg) was separated by SFC (supercritical fluid chromatography) on a CHIRALCEL® OD column using liquid CO₂ and MeOH [0.1% aqueous NH₃ as modifier] (60:40) to give trans-5-(2-(5-chloro-4-fluoro-2-(2-methoxyethoxy)phenyl)cyclopropyl)-2,2′-bipyrimidine (single enantiomer I) as a white solid (faster eluting enantiomer, 32 mg, 36% yield, m/z: 401 [M+H]⁺ observed), and trans-5-(2-(5-chloro-4-fluoro-2-(2-methoxyethoxy)phenyl)cyclopropyl)-2,2′-bipyrimidine (single enantiomer II) as a white solid (slower eluting enantiomer, 28mg, 30% yield, m/z: 401 [M+H]⁺ observed).

### Example 156: trans-5-(2-(5-Chloro-4-fluoro-2-(2-methoxyethoxy)phenyl)cyclopropyl)-2,2′-bipyrimidine (single enantiomer I)

m/z: 401 [M+H]⁺ observed. ¹H NMR (400 MHz, DMSO-d₆): δ 8.99 (d, J=4.8 Hz, 2H), 8.86 (s, 2H), 7.62 (t, J=4.8 Hz, 1H), 7.30 (d, J=8.4 Hz, 1H), 7.15 (d, J=11.2 Hz, 1H), 4.13 (t, J=4 Hz, 2H), 3.61-3.56 (m, 2H), 3.14 (s, 3H), 2.44-2.40 (m, 1H), 2.23-2.19 (m, 1H), 1.76-1.71 (m, 1H), 1.68-1.63 (m, 1H).

### Example 157: trans-5-(2-(5-Chloro-4-fluoro-2-(2-methoxyethoxy)phenyl)cyclopropyl)-2,2′-bipyrimidine (single enantiomer II)

m/z: 401 [M+H]⁺ observed. ¹H NMR (400 MHz, DMSO-d₆): δ 8.99 (d, J=4.8 Hz, 2H), 8.86 (s, 2H), 7.62 (t, J=4.8 Hz, 1H), 7.30 (d, J=8.4 Hz, 1H), 7.15 (d, J=11.2 Hz, 1H), 4.13 (t, J=4 Hz, 2H), 3.61-3.56 (m, 2H), 3.14 (s, 3H), 2.44-2.40 (m, 1H), 2.23-2.19 (m, 1H), 1.76-1.71 (m, 1H), 1.68-1.63 (m, 1H).

### Example 158: trans-5-(2-(5-Chloro-4-methoxy-[1,1′-biphenyl]-2-yl)cyclopropyl)-2,2′-bipyrimidine (single enantiomer I)

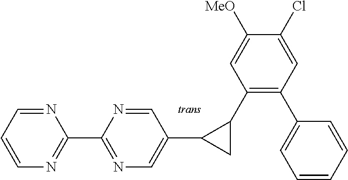 [SMILES: COc1cc(C2CC2c2cnc(-c3ncccn3)nc2)c(-c2ccccc2)cc1Cl]

### Example 159: trans-5-(2-(5-Chloro-4-methoxy-[1,1′-biphenyl]-2-yl)cyclopropyl)-2,2′-bipyrimidine (single enantiomer II)

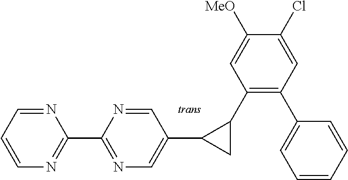 [SMILES: COc1cc(C2CC2c2cnc(-c3ncccn3)nc2)c(-c2ccccc2)cc1Cl]

A mixture of enantiomers of trans-5-(2-(5-chloro-4-methoxy-[1,1′-biphenyl]-2-yl)cyclopropyl)-2,2′-bipyrimidine (60 mg) was separated by SFC (supercritical fluid chromatography) on a CHIRALCEL® OD column using liquid CO₂ and EtOH [0.1% aqueous NH₃ modifier] (40:60) to give trans-5-(2-(5-chloro-4-methoxy-[1,1′-biphenyl]-2-yl)cyclopropyl)-2,2′-bipyrimidine (single enantiomer I) as a gray solid (faster eluting enantiomer, 22 mg, 37% yield, m/z: 415 [M+H]⁺ observed) and trans-5-(2-(5-chloro-4-methoxy-[1,1′-biphenyl]-2-yl)cyclopropyl)-2,2′-bipyrimidine (single enantiomer II) as a brown solid (slower eluting enantiomer, 23 mg, 37% yield, m/z: 415 [M+H]⁺ observed).

### Example 158: trans-5-(2-(5-Chloro-4-methoxy-[1,1′-biphenyl]-2-yl)cyclopropyl)-2,2′-bipyrimidine (single enantiomer I)

m/z: 415 [M+H]⁺ observed. ¹H NMR (400 MHz, DMSO-d₆): δ 8.98 (d, J=4.8 Hz, 2H), 8.56 (s, 2H), 7.62 (t, J=4.8 Hz, 1H), 7.30-7.29 (m, 2H), 7.28 (s, 1H), 7.22-7.20 (m, 3H), 6.95 (s, 1H), 3.96 (s, 3H), 2.32-2.29 (m, 1H), 2.17-2.15 (m, 1H), 1.84-1.82 (m, 1H), 1.65-1.62 (m, 1H).

### Example 159: trans-5-(2-(5-Chloro-4-methoxy-[1,1′-biphenyl]-2-yl)cyclopropyl)-2,2′-bipyrimidine (single enantiomer II)

m/z: 415 [M+H]⁺ observed. ¹H NMR (400 MHz, DMSO-d₆): δ 8.98 (d, J=4.8 Hz, 2H), 8.56 (s, 2H), 7.62 (t, J=4.8 Hz, 1H), 7.30-7.29 (m, 2H), 7.28 (s, 1H), 7.22-7.20 (m, 3H), 6.95 (s, 1H), 3.96 (s, 3H), 2.32-2.29 (m, 1H), 2.17-2.15 (m, 1H), 1.84-1.82 (m, 1H), 1.65-1.62 (m, 1H).

### Example 160: trans-5-(2-(3,4,5-Trifluorophenyl)cyclopropyl)-2,2′-bipyrimidine

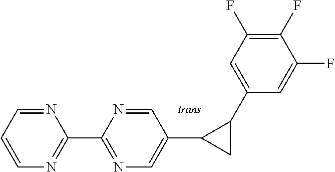 [SMILES: Fc1cc(C2CC2c2cnc(-c3ncccn3)nc2)cc(F)c1F]

m/z: 329 [M+H]⁺ observed. ¹H NMR (400 MHz, CDCl₃): δ 9.00 (d, J=4.9 Hz, 2H), 8.75 (d, J=0.4 Hz, 2H), 7.42 (t, J=4.8 Hz, 1H), 6.83-6.70 (m, 2H), 2.29 (ddd, J=8.9, 6.1, 4.5 Hz, 1H), 2.19 (ddd, J=9.0, 6.0, 4.6 Hz, 1H), 1.68-1.55 (m, 2H).

### Example 161: trans-5-(2-(3,4,5-Trifluorophenyl)cyclopropyl)-2,2′-bipyrimidine (single enantiomer I)

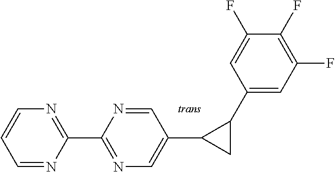 [SMILES: Fc1cc(C2CC2c2cnc(-c3ncccn3)nc2)cc(F)c1F]

### Example 162: trans-5-(2-(3,4,5-Trifluorophenyl)cyclopropyl)-2,2′-bipyrimidine (single enantiomer II)

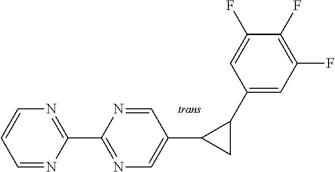 [SMILES: Fc1cc(C2CC2c2cnc(-c3ncccn3)nc2)cc(F)c1F]

A mixture of enantiomers of trans-5-(2-(3,4,5-trifluoro phenyl)cyclopropyl)-2,2′-bipyrimidine (450 mg) was separated by SFC (supercritical fluid chromatography) on a CHIRALCEL® IG column using liquid CO₂ and EtOH [0.1% aqueous NH₃ modifier] (55:45) to give trans-5-(2-(3,4,5-trifluoro phenyl)cyclopropyl)-2,2′-bipyrimidine (single enantiomer I) as a white solid (faster eluting enantiomer, 131 mg, 28% yield, m/z: 329 [M+H]⁺ observed) and trans-5-(2-(3,4,5-trifluoro phenyl)cyclopropyl)-2,2′-bipyrimidine (single enantiomer II) (slower eluting enantiomer, 63 mg, 14% yield, m/z: 329 [M+H]⁺ observed).

### Example 161: trans-5-(2-(3,4,5-Trifluorophenyl)cyclopropyl)-2,2′-bipyrimidine (single enantiomer I)

m/z: 329 [M+H]⁺ observed. ¹H NMR (400 MHz, DMSO-d₆): δ 8.99 (d, J=4.8 Hz, 2 H), 8.84 (s, 2H), 7.63 (t, J=5.2 Hz, 1H), 7.28-7.24 (m, 2H), 2.57-2.54 (m, 1 H), 2.43-2.41 (m, 1 H), 1.80-1.78 (m, 1 H), 1.71-1.69 (m, 1H).

### Example 162: trans-5-(2-(3,4,5-Trifluorophenyl)cyclopropyl)-2,2′-bipyrimidine (single enantiomer II)

m/z: 329 [M+H]⁺ observed. ¹H NMR (400 MHz, DMSO-d₆): δ 8.99 (d, J=4.8 Hz, 2 H), 8.84 (s, 2H), 7.63 (t, J=5.2 Hz, 1H), 7.28-7.24 (m, 2H), 2.57-2.54 (m, 1 H), 2.43-2.41 (m, 1 H), 1.80-1.78 (m, 1 H), 1.71-1.69 (m, 1H).

### Example 163: trans-5-(2-(3-Methoxy-5-(trifluoromethyl)phenyl)cyclopropyl)-2,2′-bipyrimidine

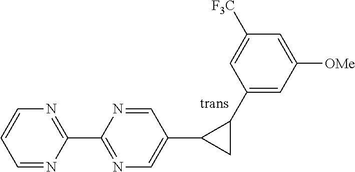 [SMILES: COc1cc(C2CC2c2cnc(-c3ncccn3)nc2)cc(C(F)(F)F)c1]

m/z: 373 [M+H]⁺ observed. ¹H NMR (400 MHz, CDCl₃): δ 9.02 (d, J=4.8 Hz, 2H), 8.79 (s, 2H), 7.43 (t, J=4.8 Hz, 1H), 7.01-6.98 (m, 2H), 6.90-6.87 (m, 1H), 3.86 (s, 3H), 2.43-2.34 (m, 1H), 2.33-2.24 (m, 1H), 1.74-1.64 (m, 2H).

### Example 164: trans-5-(2-(3-Methoxy-5-(trifluoromethoxy)phenyl)cyclopropyl)-2,2′-bipyrimidine

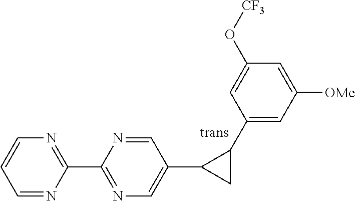 [SMILES: COc1cc(OC(F)(F)F)cc(C2CC2c2cnc(-c3ncccn3)nc2)c1]

m/z: 389 [M+H]⁺ observed. ¹H NMR (400 MHz, CDCl₃): δ 9.02 (d, J=4.8 Hz, 2H), 8.78 (s, 2H), 7.43 (t, J=4.8 Hz, 1H), 6.68-6.58 (m, 3H), 3.82 (s, 3H), 2.38-2.29 (m, 1H), 2.30-2.20 (m, 1H), 1.66 (t, J=7.5 Hz, 2H).

### Example 165: trans-5-(2-(4-Fluoro-3-methoxy-5-(trifluoromethyl)phenyl)cyclopropyl)-2,2′-bipyrimidine

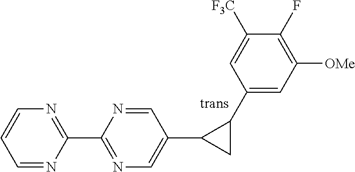 [SMILES: COc1cc(C2CC2c2cnc(-c3ncccn3)nc2)cc(C(F)(F)F)c1F]

m/z: 391 [M+H]⁺ observed. ¹H NMR (400 MHz, CDCl₃): δ 9.02 (d, J=4.8 Hz, 2H), 8.79 (s, 2H), 7.44 (t, J=4.8 Hz, 1H), 6.98 (dd, J=7.5, 2.2 Hz, 1H), 6.91 (dd, J=5.5, 2.1 Hz, 1H), 3.94 (s, 3H), 2.43-2.33 (m, 1H), 2.30-2.20 (m, 1H), 1.72-1.63 (m, 2H).

### Example 166: trans-5-(2-(Naphthalen-1-yl)cyclopropyl)-2,2′-bipyrimidine

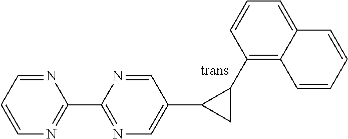 [SMILES: c1cnc(-c2ncc(C3CC3c3cccc4ccccc34)cn2)nc1]

m/z: 325 [M+H]⁺ observed. ¹H NMR (400 MHz, CDCl₃): δ 9.04 (d, J=4.9 Hz, 2H), 8.90 (s, 2H), 8.05-8.01 (m, 1H), 7.91-7.86 (m, 1H), 7.79 (d, J=8.1 Hz, 1H), 7.55-7.42 (m, 4H), 7.39 (d, J=7.1 Hz, 1H), 2.85-2.78 (m, 1H), 2.18 (dt, J=8.7, 5.3 Hz, 1H), 1.89 (dt, J=8.7, 6.0 Hz, 1H), 1.77 (dt, J=8.8, 5.6 Hz, 1H).

### Example 167: trans-5-(2-(Naphthalen-2-yl)cyclopropyl)-2,2′-bipyrimidine

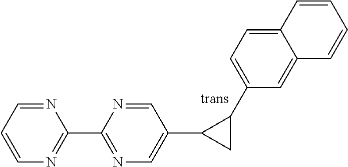 [SMILES: c1cnc(-c2ncc(C3CC3c3ccc4ccccc4c3)cn2)nc1]

m/z: 325 [M+H]⁺ observed. ¹H NMR (400 MHz, CDCl₃): δ 9.02 (d, J=4.9 Hz, 2H), 8.82 (s, 2H), 7.85-7.77 (m, 3H), 7.64 (s, 1H), 7.52-7.40 (m, 3H), 7.29 (dd, J=8.4, 1.8 Hz, 1H), 2.54 (ddd, J=9.0, 6.0, 4.7 Hz, 1H), 2.35 (ddd, J=8.9, 5.8, 4.7 Hz, 1H), 1.81 (dt, J=8.8, 5.9 Hz, 1H), 1.71 (dt, J=8.9, 5.8 Hz, 1H).

### Example 168: trans-5-(2-(4-Fluoronaphthalen-1-yl)cyclopropyl)-2,2′-bipyrimidine

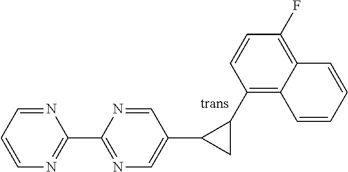 [SMILES: Fc1ccc(C2CC2c2cnc(-c3ncccn3)nc2)c2ccccc12]

m/z: 343 [M+H]+ observed. ¹H NMR (400 MHz, CDCl₃): δ 9.04 (d, J=4.8 Hz, 2H), 8.90 (s, 2H), 8.15 (d, J=7.3 Hz, 1H), 8.02 (d, J=8.1 Hz, 1H), 7.61-7.52 (m, 2H), 7.45 (t, J=4.9 Hz, 1H), 7.34-7.29 (m, 1H), 7.10 (dd, J=10.3, 7.9 Hz, 1H), 2.79-2.72 (m, 1H), 2.18-2.12 (m, 1H), 1.85 (dt, J=8.8, 6.0 Hz, 1H), 1.75 (dt, J=8.7, 5.5 Hz, 1H).

### Example 169: trans-5-(2-(4-Fluoronaphthalen-2-yl)cyclopropyl)-2,2′-bipyrimidine

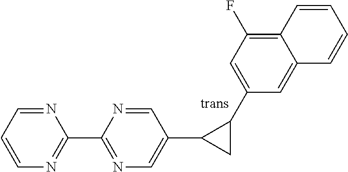 [SMILES: Fc1cc(C2CC2c2cnc(-c3ncccn3)nc2)cc2ccccc12]

m/z: 343 [M+H]⁺ observed. ¹H NMR (400 MHz, CDCl₃): δ 9.02 (d, J=4.8 Hz, 2H), 8.82 (s, 2H), 8.09-8.02 (m, 1H), 7.84-7.77 (m, 1H), 7.58-7.45 (m, 2H), 7.46 (s, 1H), 7.43 (t, J=4.8 Hz, 1H), 6.95 (dd, J=11.5, 1.6 Hz, 1H), 2.57-2.47 (m, 1H), 2.39-2.30 (m, 1H), 1.83-1.67 (m, 2H).

### Example 170: trans-3-(2-([2,2′-Bipyrimidin]-5-yl)cyclopropyl)imidazo[1,2-a]pyridine

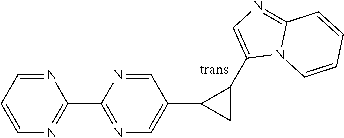 [SMILES: c1cnc(-c2ncc(C3CC3c3cnc4ccccn34)cn2)nc1]

m/z: 315 [M+H]⁺ observed. ¹H NMR (400 MHz, CDCl₃) δ 9.04 (d, J=4.8 Hz, 2H), 8.88 (s, 2H), 8.02 (d, J=6.8 Hz, 1H), 7.66 (d, J=9.1 Hz, 1H), 7.52 (s, 1H), 7.45 (t, J=4.9 Hz, 1H), 7.31-7.18 (m, 1H), 6.88 (t, J=6.8 Hz, 1H), 2.40-2.33 (m, 1H), 2.29-2.22 (m, 1H), 1.82-1.74 (m, 2H).

### Example 171: trans-5-(2-([2,2′-Bipyrimidin]-5-yl)cyclopropyl)quinoline

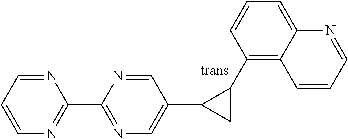 [SMILES: c1cnc(-c2ncc(C3CC3c3cccc4ncccc34)cn2)nc1]

m/z: 326 [M+H]⁺ observed. ¹H NMR (400 MHz, CDCl₃): δ 9.05 (d, J=4.9 Hz, 2H), 8.96 (dd, J=4.2, 1.7 Hz, 1H), 8.90 (s, 2H), 8.40-8.36 (m, 1H), 8.05 (d, J=8.5 Hz, 1H), 7.69 (dd, J=8.5, 7.1 Hz, 1H), 7.49-7.38 (m, 3H), 2.84-2.77 (m, 1H), 2.22 (dt, J=8.8, 5.3 Hz, 1H), 1.89 (dt, J=8.7, 5.9 Hz, 1H), 1.80 (dt, J=8.9, 5.6 Hz, 1H).

### Example 172: trans-5-(2-([2,2′-Bipyrimidin]-5-yl)cyclopropyl)-8-fluoroquinoline

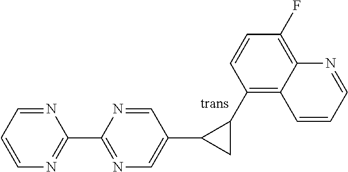 [SMILES: Fc1ccc(C2CC2c2cnc(-c3ncccn3)nc2)c2cccnc12]

m/z: 344 [M+H]⁺ observed. ¹H NMR (400 MHz, CDCl₃): δ 9.05 (d, J=4.8 Hz, 2H), 9.02 (dd, J=4.2, 1.6 Hz, 1H), 8.89 (s, 2H), 8.38 (dt, J=8.6, 1.6 Hz, 1H), 7.50 (dd, J=8.6, 4.2 Hz, 1H), 7.46 (t, J=4.9 Hz, 1H), 7.42-7.34 (m, 2H), 2.78-2.71 (m, 1H), 2.20 (dt, J=8.8, 5.3 Hz, 1H), 1.88-1.83 (m, 1H), 1.79 (dt, J=8.9, 5.6 Hz, 1H).

### Example 173: trans-5-(2-([2,2′-Bipyrimidin]-5-yl)cyclopropyl)-8-methoxyquinoline

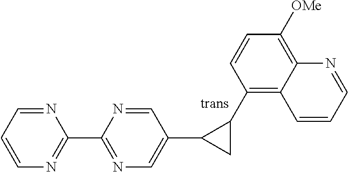 [SMILES: COc1ccc(C2CC2c2cnc(-c3ncccn3)nc2)c2cccnc12]

m/z: 356 [M+H]⁺ observed. ¹H NMR (400 MHz, CDCl₃): δ 9.04 (d, J=4.9 Hz, 2H), 8.97 (dd, J=4.2, 1.7 Hz, 1H), 8.88 (s, 2H), 8.33 (dd, J=8.5, 1.7 Hz, 1H), 7.48-7.43 (m, 2H), 7.38 (dd, J=8.0, 0.8 Hz, 1H), 7.00 (d, J=7.9 Hz, 1H), 4.10 (s, 3H), 2.73-2.66 (m, 1H), 2.15 (dt, J=8.9, 5.3 Hz, 1H), 1.86-1.79 (m, 1H), 1.74 (dt, J=8.8, 5.5 Hz, 1H).

### Example 174: trans-5-(2-(3,4-Difluoro-5-methoxyphenyl)cyclopropyl)-2-(pyridin-2-yl)pyrimidine

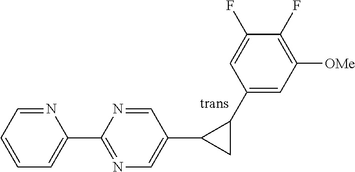 [SMILES: COc1cc(C2CC2c2cnc(-c3ccccn3)nc2)cc(F)c1F]

m/z: 340 [M+H]⁺ observed. ¹H NMR (400 MHz, CDCl₃): δ 9.12 (d, J=5.5 Hz, 1H), 8.82 (dd, J=8.1, 1.2 Hz, 1H), 8.74 (s, 2H), 8.42 (t, J=7.8 Hz, 1H), 7.89 (t, J=6.4 Hz, 1H), 6.60 (dt, J=6.8, 2.0 Hz, 1H), 6.53 (ddd, J=10.6, 6.3, 2.1 Hz, 1H), 3.92 (s, 3H), 2.36 (td, J=7.5, 4.5 Hz, 1H), 2.17 (td, J=7.4, 4.5 Hz, 1H), 1.66 (dd, J=8.1, 6.9 Hz, 2H).

### Example 175: trans-5-(2-(4-Fluoro-3,5-dimethoxyphenyl)cyclopropyl)-2-(pyridin-2-yl)pyrimidine

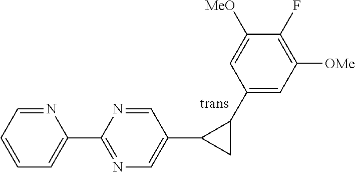 [SMILES: COc1cc(C2CC2c2cnc(-c3ccccn3)nc2)cc(OC)c1F]

m/z: 352 [M+H]⁺ observed. ¹H NMR (400 MHz, CDCl₃): δ 9.12 (ddd, J=5.7, 1.6, 0.7 Hz, 1H), 8.90 (ddd, J=8.1, 1.3, 0.7 Hz, 1H), 8.74 (d, J=3.8 Hz, 2H), 8.53 (td, J=7.9, 1.6 Hz, 1H), 7.99 (ddd, J=7.7, 5.7, 1.3 Hz, 1H), 6.41 (d, J=6.8 Hz, 2H), 3.89 (s, 6H), 2.45-2.30 (m, 1H), 2.18 (ddd, J=8.8, 5.9, 4.5 Hz, 1H), 1.68 (ddt, J=25.5, 9.0, 5.9 Hz, 2H).

### Example 176: trans-2-(Pyridin-2-yl)-5-(2-(3,4,5-trimethoxyphenyl)cyclopropyl)pyrimidine

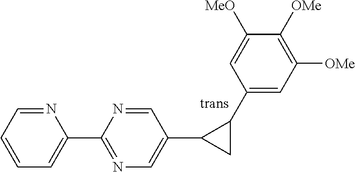 [SMILES: COc1cc(C2CC2c2cnc(-c3ccccn3)nc2)cc(OC)c1OC]

m/z: 364 [M+H]⁺ observed. ¹H NMR (400 MHz, CDCl₃): δ 9.13 (d, J=5.6 Hz, 1H), 8.87 (ddd, J=8.1, 1.4, 0.7 Hz, 1H), 8.75 (s, 2H), 8.54-8.42 (m, 1H), 7.99-7.87 (m, 1H), 6.39 (s, 2H), 3.88 (s, 6H), 3.84 (s, 3H), 2.39 (ddd, J=8.9, 6.3, 4.5 Hz, 1H), 2.24-2.14 (m, 1H), 1.69 (ddt, J=31.6, 8.9, 5.9 Hz, 2H).

### Example 177: trans-5-(2-(3,4-Difluoro-5-methoxyphenyl)cyclopropyl)-2,4′-bipyrimidine

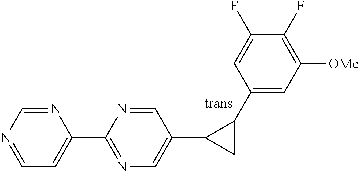 [SMILES: COc1cc(C2CC2c2cnc(-c3ccncn3)nc2)cc(F)c1F]

m/z: 341 [M+H]⁺ observed. ¹H NMR (400 MHz, CDCl₃): δ 9.45 (s, 1H), 8.95 (d, J=5.2 Hz, 1H), 8.73 (s, 2H), 8.42 (dd, J=5.2, 1.4 Hz, 1H), 6.62-6.50 (m, 2H), 3.92 (s, 3H), 2.36-2.26 (m, 1H), 2.23-2.13 (m, 1H), 1.67-1.58 (m, 2H).

### Example 178: trans-5-(2-(3,4-Difluoro-5-methoxyphenyl)cyclopropyl)-2-(pyrazin-2-yl)pyrimidine

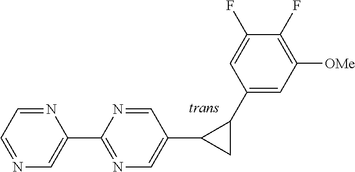 [SMILES: COc1cc(C2CC2c2cnc(-c3cnccn3)nc2)cc(F)c1F]

m/z: 341.2 [M+H]. ¹H NMR (400 MHz, CDCl₃): δ 9.71 (s, 1H), 8.77 (s, 1H), 8.75-8.66 (m, 3H), 6.62-6.50 (m, 2H), 3.91 (s, 3H), 2.34-2.24 (m, 1H), 2.22-2.12 (m, 1H), 1.66-1.56 (m, 2H).

### Example 179: trans-5-(2-(3,4-Difluoro-5-methoxyphenyl)cyclopropyl)-2-(3-fluoropyridin-2-yl)pyrimidine

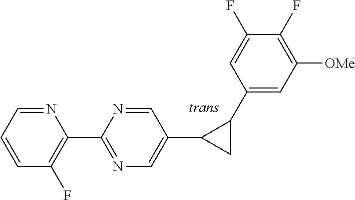 [SMILES: COc1cc(C2CC2c2cnc(-c3ncccc3F)nc2)cc(F)c1F]

m/z: 358 [M+H]⁺ observed. ¹H NMR (400 MHz, CDCl₃): δ 8.73 (s, 2H), 8.64-8.63 (m, 1H), 7.65-7.55 (m, 1H), 7.51-7.39 (m, 1H), 6.63-6.50 (m, 2H), 3.92 (s, 3H), 2.33-2.23 (m, 1H), 2.21-2.11 (m, 1H), 1.66-1.55 (m, 2H).

### Example 180: trans-5-(2-(3,4-Difluoro-5-methoxyphenyl)cyclopropyl)-2-(3-methoxypyridin-2-yl)pyrimidine

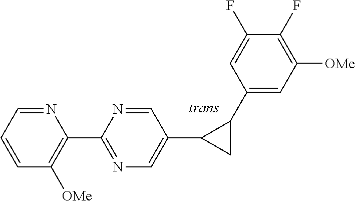 [SMILES: COc1cccnc1-c1ncc(C2CC2c2cc(F)c(F)c(OC)c2)cn1]

m/z: 370 [M+H]⁺ observed. ¹H NMR (400 MHz, CDCl₃): δ 8.70 (s, 2H), 8.39-8.37 (m, 1H), 7.41-7.36 (m, 2H), 6.62-6.49 (m, 2H), 3.92 (s, 3H), 3.90 (s, 3H), 2.31-2.21 (m, 1H), 2.20-2.10 (m, 1H), 1.65-1.51 (m, 2H).

### Example 181: trans-5-(2-(3,4-Difluoro-5-(1H-imidazol-1-yl)phenyl)cyclopropyl)-2,2′-bipyrimidine (single enantiomer I)

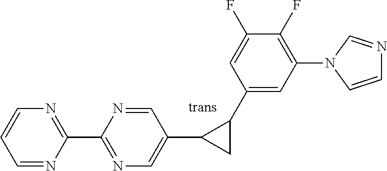 [SMILES: Fc1cc(C2CC2c2cnc(-c3ncccn3)nc2)cc(-n2ccnc2)c1F]

### Example 182: trans-5-(2-(3,4-Difluoro-5-(1H-imidazol-1-yl)phenyl)cyclopropyl)-2,2′-bipyrimidine (single enantiomer II)

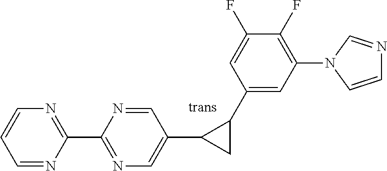 [SMILES: Fc1cc(C2CC2c2cnc(-c3ncccn3)nc2)cc(-n2ccnc2)c1F]

### (E)-4,4,5,5-Tetramethyl-2-(3,4,5-trifluorostyryl)-1,3,2-dioxaborolane

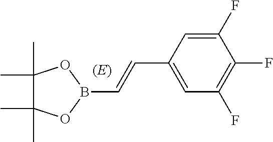 [SMILES: CC1(C)OB(/C=C/c2cc(F)c(F)c(F)c2)OC1(C)C]

To a solution of 5-bromo-13-trifluorobenzene (10 g, 47.6 mmol) in toluene (100 mL) was added of triethylamine (19.8 mL, 143.5 mmol) then 4,4,5,5-tetramethyl-2-vinyl-1,3,2-dioxaborolane (11 g, 71.4 mmol) was added at room temperature and the mixture was degassed with N₂ gas for 5 min. Bis(tri-tert-butylphosphine)palladium (244 mg, 0.478 mmol) was added and the mixture was heated to 120° C. for 16 h in a sealed tube. The reaction mixture was poured into ice water (200 mL) and extracted with EtOAc (2×200 mL). The organic layer was washed with saturated aqueous brine solution (100 mL), dried over Na₂SO₄, filtered, and evaporated under reduced pressure. The residue was washed with n-pentane (2×20 mL) then dried to give (E)-4,4,5,5-tetramethyl-2-(3,4,5-trifluorostyryl)-1,3,2-dioxaborolane as a brown liquid, which was used in the next step without further purification (7.0 g, 51% yield). ¹H NMR (400 MHz, CDCl₃): δ 7.19 (d, 1H), 7.10-7.04 (m, 2H), 6.07 (d, 1H), 1.30 (s, 12H).

### trans-4,4,5,5-Tetramethyl-2-(2-(3,4,5-trifluorophenyl)cyclopropyl)-1,3,2-dioxaborolane

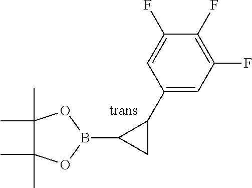 [SMILES: CC1(C)OB(C2CC2c2cc(F)c(F)c(F)c2)OC1(C)C]

To a solution of (E)-4,4,5,5-tetramethyl-2-(3,4,5-trifluorostyryl)-1,3,2-dioxaborolane (7.0 g, 25 mmol) in THF (70 mL) at 0° C. was added Pd(OAc)₂ (trimer, 559 mg, 2.46 mmol) and freshly prepared ethereal diazomethane [prepared from N-methyl-N-nitroso urea (50.8 g, 492 mmol), KOH solution (50% in H₂O, 500 mL) and Et₂O (500 mL) at 0° C.] and the reaction mixture was stirred at 0° C. for 4 h. The reaction mixture was filtered through a pad of CELITE® and filtrate was evaporated. The cyclopropanation process was repeated twice until complete product formation was observed. The residue was purified by normal phase SiO₂ chromatography (0-20% EtOAc/petroleum ether) to afford trans-4,4,5,5-tetramethyl-2-(2-(3,4,5-trifluorophenyl)cyclopropyl)-1,3,2-dioxaborolane as a yellow solid (6.0 g, 80% yield). ¹H NMR (400 MHz, CDCl₃): δ 0.68-6.64 (m, 2H), 2.04-1.99 (m, 1H), 1.24 (s, 12H), 1.17-1.14 (m, 1H), 0.94-0.90 (m, 1H), 0.24-0.21(m, 1H).

### trans-Trifluoro(2-(3,4,5-trifluorophenyl)cyclopropyl)-λ⁴-borane, potassium salt

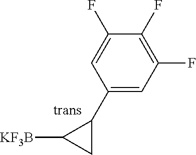

To a solution of trans-4,4,5,5-tetramethyl-2-(2-(3,4,5-trifluorophenyl)cyclopropyl)-1,3,2-dioxaborolane (6.0 g, 20 mmol) in MeOH-H₂O (8:2, 60 mL) was added KHF₂ (11 g, 141 mmol) at room temperature. The reaction mixture was heated at 80° C. for 16 h. The solvent was evaporated under reduced pressure. The residue was dissolved in MeCN (200 mL), filtered through CELITE® and the filtrate was evaporated under reduced pressure. The residue was triturated with n-hexane (3×50 mL) to afford trans-trifluoro(2-(3,4,5-trifluorophenyl)cyclopropyl)-λ⁴-borane, potassium salt as white solid (5.5 g, 89% yield). ¹H NMR (400 MHz, DMSO-d₆) δ 6.84-6.80 (m, 2H), 1.50-1.46 (m, 1.10-1.06 (m, 1H), 0.67-0.66 (d, 1H), 0.40-0.38 (d, 1H).

### trans-2-Chloro-5-(2-(3,4, 5-trifluorophenyl)cyclopropyl)pyrimidine

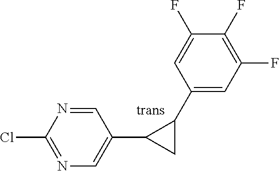 [SMILES: Fc1cc(C2CC2c2cnc(Cl)nc2)cc(F)c1F]

To a solution of trans-trifluoro(2-(3,4,5-trifluorophenyl)cyclopropyl)-λ⁴-borane, potassium salt (5.5 g, 19.8 mmol) in 1,4-dioxane-water (9:1, 55 mL) was added 5-bromo-2-chloropyridine (5.70, 29.5 mmol) of and Cs₂CO₃ (12.9 g, 40.0 mmol) at room temperature and the mixture was purged with N₂ gas for 10 min followed by addition of Pd(dppf)Cl₂ (1.44 g, 1.97 mmol) and the purging with N₂ gas was continued for 10 min. The reaction mixture was heated at 100° C. for 16 h in a sealed tube. The reaction mixture was cooled to room temperature, filtered through CELITE®, and the filtrate was evaporated under reduced pressure. The residue was purified by normal phase SiO₂ chromatography (0-30% EtOAc/petroleum ether) to afford trans-2-chloro-5-(2-(3,4,5-trifluorophenyl)cyclopropyl)pyrimidine as yellow solid (1.6 g, 28% yield, m/z: 285 [M+H]⁺ observed). ¹H NMR (400 MHz, CDCl₃): δ 8.41 (s, 2H), 6.77-6.74 (m, 2H), 2.21-2.16 (m, 1H), 2.09-2.06 (m, 1H), 1.68-1.54 (m, 2H).

### trans-5-(2-(3,4,5-Trifluorophenyl)cyclopropyl)-2,2′-bipyrimidine

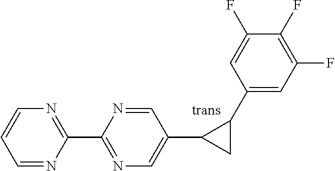 [SMILES: Fc1cc(C2CC2c2cnc(-c3ncccn3)nc2)cc(F)c1F]

To a solution of trans-2-chloro-5-(2-(3,4,5-trifluorophenypcyclopropyppyrimidine (1.6 g, 5.6 mmol) in 1,4-dioxane (15 mL) was added 2-(tributylstannyl)pyrimidine (1.8 mL, 5.6 mmol) and Cu' (110 mg, 0.57 mmol) of at room temperature and the mixture was purged with N₂ gas for 10 min, followed by the addition of PdCl₂(PPh₃)₂ (393 mg, 0.56 mmol). The purging with N₂ gas was continued for 10 min. The reaction mixture was stirred at 120° C. for 48 h in a sealed tube. The reaction mixture was cooled to room temperature, filtered through CELTTE® and the filtrate was concentrated under vacuum. The residue was purified by normal phase SiO₂ chromatography (0-3% MeOH/CH₂Cl₂) to afford trans-5-(2-(3,4,5-trifluorophenyl)cyclopropyl)-2,2° -bipyrimidine as a white solid (0.60 g, 32% yield, m/z: 329 [M+H]⁺ observed). ¹H NMR (400 MHz, CDCl₃): δ 9.02 (d, 2H), 8.77 (s, 2H), 7.43 (t, 1H), 6.81-6.78 (m, 2H), 2.30-2.29 (m, 1H), 2.21-2.19 (m, 1H), 1.68-1.54 (m,

### trans-5-(2-(3,4-Difluoro-5-(1H-imidazol-1-yl)phenyl)cyclopropyl)-2,2′-bipyrimidine

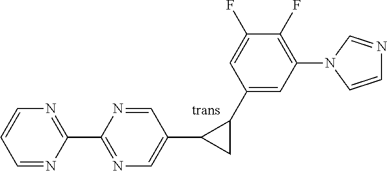 [SMILES: Fc1cc(C2CC2c2cnc(-c3ncccn3)nc2)cc(-n2ccnc2)c1F]

To a solution of trans-5-(2-(3,4,5-trifluorophenyl)cyclopropyl)-2,2′-bipyrimidine (0.60 g, 1.8 mmol) in DMSO (6 mL) was added K₂CO₃(0.25 g, 1.8 mmol) and imidazole (0.12 g, 1.8 mmol). The reaction mixture was stirred and heated at 120° C. for 16 h. The reaction mixture was poured into ice-water (20 mL) and extracted with CH₂Cl₂ (2×20 mL). The organic layer was washed with saturated aqueous brine solution (10 mL), dried over Na₂SO₄, and evaporated under reduced pressure. The residue was purified by normal phase SiO₂ chromatography (0-6% MeOH/CH₂Cl₂) to afford trans-5-(2-(3,4-difluoro-5-(1H-imidazol-1-yl)phenyl)cyclopropyl)-2,2′-bipyrimidine as an off-white solid (70 mg, 10% yield, m/z: 377 [M+H]⁺ observed).

A mixture of enantiomers of trans-5-(2-(3,4-difluoro-5-(1H-imidazol-1-yl)phenyl)cyclopropyl)-2,2′-bipyrimidine (70 mg) was separated by SFC (supercritical fluid chromatography) on a CHIRALCEL® OJ-H column using liquid CO₂ and [7M Ammonia in MeOH] (78:22) to give trans-5-(2-(3,4-difluoro-5-(1H-imidazol-1-yl)phenyl)cyclopropyl)-2,2′-bipyrimidine (single enantiomer I) as an off-white solid (faster eluting enantiomer, 8 mg, 11% yield , m/z: 377 [M+H]⁺ observed), and trans-5-(2-(3,4-difluoro-5-(1H-imidazol-1-yl)phenyl)cyclopropyl)-2,2′-bipyrimidine (single enantiomer II) as an off-white solid (slower eluting enantiomer, 8 mg, 11% yield, m/z: 377 [M+H]⁺ observed).

### Example 181: trans-5-(2-(3,4-Difluoro-5-(1H-imidazol-1-yl)phenyl)cyclopropyl)-2,2′-bipyrimidine (single enantiomer I)

m/z: 377 [M+H]⁺ observed. ¹H NMR (400 MHz, DMSO-d₆): δ 8.99 (d, 2H), 8.85 (s, 2H), 8.11 (s, 1H) 7.64-7.61 (m, 2H), 7.47-7.38 (m, 2H), 7.15 (s, 1H), 2.62-2.57 (m, 2H), 1.84-1.76 (m, 2H).

### Example 182: trans-5-(2-(3,4-Difluoro-5-(1H-imidazol-1-yl)phenyl)cyclopropyl)-2,2′-bipyrimidine (single enantiomer II)

m/z: 377 [M+H]⁺ observed. ¹H NMR (400 MHz, DMSO-d₆): δ 8.99 (d, 2H), 8.85 (s, 2H), 8.11 (s, 1H) 7.64-7.61 (m, 2H), 7.47-7.38 (m, 2H), 7.15 (s, 1H), 2.62-2.57 (m, 2H), 1.84-1.76 (m, 2H).

The following examples were prepared in a similar manner as trans-5-(2-(3,4-Difluoro-5-(1H-imidazol-1-yl)phenyl)cyclopropyl)-2,2′-bipyrimidine from and appropriately substituted aryl bromide and 4,4,5,5-tetramethyl-2-vinyl-1,3,2-dioxaborolane:

### Example 183: trans-5-(2-(5,6-Dimethoxypyridin-3-yl)cyclopropyl)-2,2′-bipyrimidine (single enantiomer I)

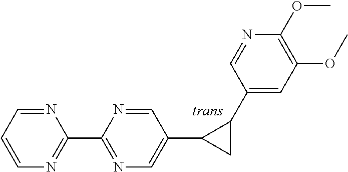 [SMILES: COc1cc(C2CC2c2cnc(-c3ncccn3)nc2)cnc1OC]

### Example 184: trans-5-(2-(5,6-Dimethoxypyridin-3-yl)cyclopropyl)-2,2′-bipyrimidine (single enantiomer II)

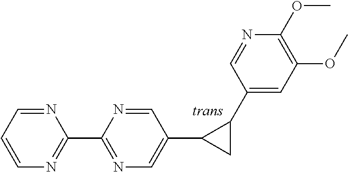 [SMILES: COc1cc(C2CC2c2cnc(-c3ncccn3)nc2)cnc1OC]

A mixture of enantiomers of trans-5-(2-(5,6-dimethoxypyridin-3-yl)cyclopropyl)-2,2′-bipyrimidine (36 mg) was separated by SFC on a CHIRALPAK® AD-H column using liquid CO₂ and 30 mM Methanolic ammonia in EtOH (55:45) to give trans-5-(2-(5,6-dimethoxypyridin-3-yl)cyclopropyl)-2,2′-bipyrimidine (single enantiomer I) as a brown solid (faster eluting enantiomer, 12 mg, 33% yield, m/z: 336 [M+H]⁺ observed) and trans-5-(2-(5,6-dimethoxypyridin-3-yl)cyclopropyl)-2,2′-bipyrimidine (single enantiomer II) as a brown solid (slower eluting enantiomer, 13 mg, 36% yield, m/z: 336 [M+H]⁺ observed).

### Example 183: trans-5-(2-(5,6-Dimethoxypyridin-3-yl)cyclopropyl)-2,2′-bipyrimidine (single enantiomer I)

m/z: 336 [M+H]⁺ observed. ¹H NMR (400 MHz, DMSO-d₆): δ 8.99 (d, 2H), 8.85 (s, 2H), 7.64-7.61 (m, 2H), 7.10 (d, 1H), 3.83 (s, 3H), 3.80 (s, 3H), 2.51-2.49 (m, 1H), 2.38-2.36 (m, 1H), 1.71-1.67 (m, 2H).

### Example 184: trans-5-(2-(5,6-Dimethoxypyridin-3-yl)cyclopropyl)-2,2′-bipyrimidine (single enantiomer II)

m/z: 336 [M+H]⁺ observed. ¹H NMR (400 MHz, DMSO-d₆): δ 8.99 (d, 2H), 8.85 (s, 2H), 7.64-7.61 (m, 2H), 7.10 (d, 1H), 3.83 (s, 3H), 3.80 (s, 3H), 2.51-2.49 (m, 1H), 2.38-2.36 (m, 1H), 1.71-1.67 (m, 2H).

### Example 185: trans-5-(2-(3-(Difluoromethoxy)-4-fluorophenyl)cyclopropyl)-2,2′-bipyrimidine (single enantiomer I)

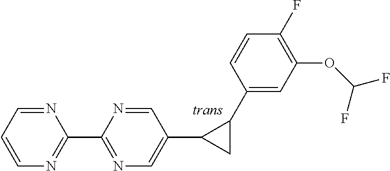 [SMILES: Fc1ccc(C2CC2c2cnc(-c3ncccn3)nc2)cc1OC(F)F]

### Example 186: trans-5-(2-(3-(Difluoromethoxy)-4-fluorophenyl)cyclopropyl)-2,2′-bipyrimidine (single enantiomer II)

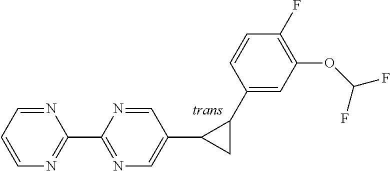 [SMILES: Fc1ccc(C2CC2c2cnc(-c3ncccn3)nc2)cc1OC(F)F]

A mixture of enantiomers of trans-5-(2-(3-(difluoromethoxy)-4-fluorophenyl)cyclopropyl)-2,2′-bipyrimidine (60 mg) was separated by HPLC on a CHIRALPAK® OJ-H column using n-hexanes and EtOH (60:40) to give trans-5-(2-(3-(difluoromethoxy)-4-fluorophenyl)cyclopropyl)-2,2′-bipyrimidine (single enantiomer I) as a brown solid (faster eluting enantiomer, 8 mg, 13% yield, m/z: 359 [M+H]⁺ observed) and trans-5-(2-(3-(difluoromethoxy)-4-fluorophenyl)cyclopropyl)-2,2′-bipyrimidine (single enantiomer II) as a brown solid (slower eluting enantiomer, 9 mg, 15% yield, m/z: 359 [M+H]⁺ observed).

### Example 185: trans-5-(2-(3-(Difluoromethoxy)-4-fluorophenyl)cyclopropyl)-2,2′-bipyrimidine (single enantiomer I)

m/z: 359 [M+H]⁺ observed. ¹H NMR (400 MHz, DMSO-d₆): δ 8.99 (d, 2H), 8.85 (s, 2H), 7.63-7.61 (m, 1H), 7.44-7.07 (m, 4H), 2.67-2.56 (m, 1H), 2.40-2.35 (m, 1H), 1.77-1.72 (m, 1H), 1.68-1.64 (m, 1H).

### Example 186: trans-5-(2-(3-(Difluoromethoxy)-4-fluorophenyl)cyclopropyl)-2,2′-bipyrimidine (single enantiomer II)

m/z: 359 [M+H]⁺ observed. ¹H NMR (400 MHz, DMSO-d₆): δ 8.99 (d, 2H), 8.85 (s, 2H), 7.63-7.61 (m, 1H), 7.44-7.07 (m, 4H), 2.67-2.56 (m, 1H), 2.40-2.35 (m, 1H), 1.77-1.72 (m, 1H), 1.68-1.64 (m, 1H).

### Example 187: trans-5-(2-(4-Fluoro-3-(4-methylpiperazin-1-yl)phenyl)cyclopropyl)-2,2′-bipyrimidine (single enantiomer I)

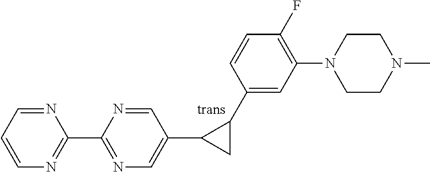 [SMILES: CN1CCN(c2cc(C3CC3c3cnc(-c4ncccn4)nc3)ccc2F)CC1]

### Example 188: trans-5-(2-(4-Fluoro-3-(4-methylpiperazin-1-yl)phenyl)cyclopropyl)-2,2′-bipyrimidine (single enantiomer II)

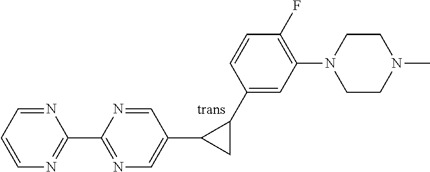 [SMILES: CN1CCN(c2cc(C3CC3c3cnc(-c4ncccn4)nc3)ccc2F)CC1]

A mixture of trans-5-(2-(4-fluoro-3-(4-methylpiperazin-1-yl)phenyl)cyclopropyl)-2,2′-bipyrimidine (90 mg) was separated by HPLC on a CHIRALPAK® AD-H column using n-hexanes and EtOH (30:70) to give trans-5-(2-(4-fluoro-3-(4-methylpiperazin-1-yl)phenyl)cyclopropyl)-2,2′-bipyrimidine (single enantiomer I) as an off-white solid (faster eluting enantiomer, 15 mg, 17% yield, m/z: 391 [M+H]⁺ observed) and trans-5-(2-(4-fluoro-3-(4-methylpiperazin-1-yl)phenyl)cyclopropyl)-2,2′-bipyrimidine (single enantiomer II) as an off-white solid (slower eluting enantiomer, 19 mg, 21% yield, m/z: 391 [M+H]⁺ observed).

### Example 187: trans-5-(2-(4-Fluoro-3-(4-methylpiperazin-1-yl)phenyl)cyclopropyl)-2,2′-bipyrimidine (single enantiomer I)

m/z: 391 [M+H]⁺ observed. ¹H NMR (400 MHz, DMSO-d₆): δ 8.99 (d, 2H), 8.84 (s, 2H), 7.62 (t, 1H), 7.14-7.09 (m, 1H), 6.96-6.94 (m, 1H), 6.88-6.84 (m, 1H), 3.56-3.30 (m, 4H), 3.26-3.05 (m, 4H), 2.78 (br s, 3H), 2.51-2.37 (m, 1H), 2.36-2.32 (m, 1H), 1.74-1.62 (m, 2H).

### Example 188: trans-5-(2-(4-Fluoro-3-(4-methylpiperazin-1-yl)phenyl)cyclopropyl)-2,2′-bipyrimidine (single enantiomer II)

m/z: 391 [M+H]⁺ observed. ¹H NMR (400 MHz, DMSO-d₆): δ 8.99 (d, 2H), 8.84 (s, 2H), 7.62 (t, 1H), 7.14-7.09 (m, 1H), 6.96-6.94 (m, 1H), 6.88-6.84 (m, 1H), 3.56-3.30 (m, 4H), 3.26-3.05 (m, 4H), 2.78 (br s, 3H), 2.51-2.37 (m, 1H), 2.36-2.32 (m, 1H), 1.74-1.62 (m, 2H).

### Example 189: trans-5-(2-(2,4-Difluoro-3-methoxyphenyl)cyclopropyl)-2,2′-bipyrimidine (single enantiomer I)

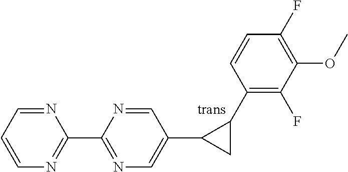 [SMILES: COc1c(F)ccc(C2CC2c2cnc(-c3ncccn3)nc2)c1F]

### Example 190: trans-5-(2-(2,4-Difluoro-3-methoxyphenyl)cyclopropyl)-2,2′-bipyrimidine (single enantiomer II)

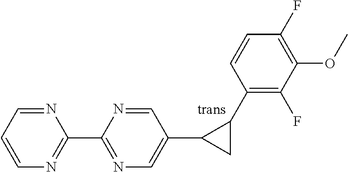 [SMILES: COc1c(F)ccc(C2CC2c2cnc(-c3ncccn3)nc2)c1F]

A mixture of trans-5-(2-(2,4-difluoro-3-methoxyphenyl)cyclopropyl)-2,2′-bipyrimidine (70 mg) was separated by SFC on a CHIRALPAK® OD-H column using liquid CO₂ and MeOH (50:50) to give trans-5-(2-(2,4-difluoro-3-methoxyphenyl)cyclopropyl)-2,2′-bipyrimidine (single enantiomer I) as an off-white solid (faster eluting enantiomer, 11 mg, 16% yield, m/z: 341 [M+H]⁺ observed) and trans-5-(2-(2,4-difluoro-3-methoxyphenyl)cyclopropyl)-2,2′-bipyrimidine (single enantiomer II) as an off-white solid (slower eluting enantiomer, 13 mg, 19% yield, m/z: 341 [M+H]⁺ observed).

### Example 189: trans-5-(2-(2,4-Difluoro-3-methoxyphenyl)cyclopropyl)-2,2′-bipyrimidine (single enantiomer I)

m/z: 341 [M+H]⁺ observed. ¹H NMR (400 MHz, DMSO-d₆): δ 8.99 (d, 2H), 8.89 (s, 2H), 7.62 (t, 1H), 7.14-7.09 (m, 1H), 7.10-6.95 (m, 1H), 3.92 (s, 3H), 2.55-2.50 (m, 1H), 2.42-2.32 (m, 1H), 1.77-1.62 (m, 2H).

### Example 190: trans-5-(2-(2,4-Difluoro-3-methoxyphenyl)cyclopropyl)-2,2′-bipyrimidine (single enantiomer II)

m/z: 341 [M+H]⁺ observed. ¹H NMR (400 MHz, DMSO-d₆): δ 8.99 (d, 2H), 8.89 (s, 2H), 7.62 (t, 1H), 7.14-7.09 (m, 1H), 7.10-6.95 (m, 1H), 3.92 (s, 3H), 2.55-2.50 (m, 1H), 2.42-2.32 (m, 1H), 1.77-1.62 (m, 2H).

### Example 191: trans-3-(2-(4-Fluoro-3-methoxyphenyl)cyclopropyl)-6-(pyrimidin-2-yl)pyridazine (single enantiomer I)

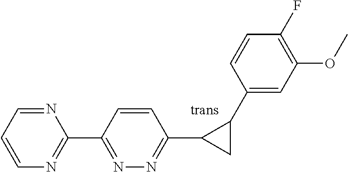 [SMILES: COc1cc(C2CC2c2ccc(-c3ncccn3)nn2)ccc1F]

### Example 192: trans-3-(2-(4-Fluoro-3-methoxyphenyl)cyclopropyl)-6-(pyrimidin-2-yl)pyridazine (single enantiomer II)

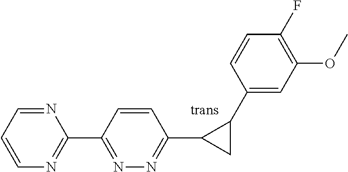 [SMILES: COc1cc(C2CC2c2ccc(-c3ncccn3)nn2)ccc1F]

A mixture of trans-3-(2-(4-fluoro-3-methoxyphenyl)cyclopropyl)-6-(pyrimidin-2-yl)pyridazine (45 mg) was separated by SFC on a CHIRALPAK® OD-H column using liquid CO₂ and 30 mM Methanolic ammonia in EtOH (60:40) to give trans-3-(2-(4-fluoro-3-methoxyphenyl)cyclopropyl)-6-(pyrimidin-2-yl)pyridazine (single enantiomer I) as a brick red solid (faster eluting enantiomer, 5 mg, 11% yield, m/z: 323 [M+H]⁺ observed) and trans-3-(2-(4-fluoro-3-methoxyphenyl)cyclopropyl)-6-(pyrimidin-2-yl)pyridazine (single enantiomer II) as a brick red solid (slower eluting enantiomer, 6 mg, 13% yield, m/z: 323 [M+H]⁺ observed).

### Example 191: trans-3-(2-(4-Fluoro-3-methoxyphenyl)cyclopropyl)-6-(pyrimidin-2-yl)pyridazine (single enantiomer I)

m/z: 323 [M+H]⁺ observed. ¹H NMR (400 MHz, DMSO-d₆): δ 9.04-9.03 (m, 2H), 8.37 (d, 1H), 7.79 (d, 1H), 7.65-7.62 (m, 1H), 7.15-7.04 (m, 2H), 6.83-6.79 (m, 1H), 3.82 (s, 3H), 2.71-2.66 (m, 2H), 1.91-1.87 (m, 1H), 1.75-1.70 (m, 1H).

### Example 192: trans-3-(2-(4-Fluoro-3-methoxyphenyl)cyclopropyl)-6-(pyrimidin-2-yl)pyridazine (single enantiomer II)

m/z: 323 [M+H]⁺ observed. ¹H NMR (400 MHz, DMSO-d₆): δ 9.04-9.03 (m, 2H), 8.37 (d, 1H), 7.79 (d, 1H), 7.65-7.62 (m, 1H), 7.15-7.04 (m, 2H), 6.83-6.79 (m, 1H), 3.82 (s, 3H), 2.71-2.66 (m, 2H), 1.91-1.87 (m, 1H), 1.75-1.70 (m, 1H).

### Example 193: trans-4-(2-(4-Fluoro-3-methoxyphenyl)cyclopropyl)-1-(pyrimidin-2-yl)isoquinoline (single enantiomer I)

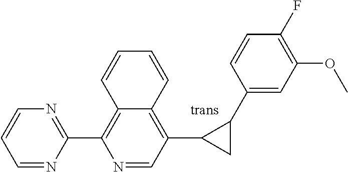 [SMILES: COc1cc(C2CC2c2cnc(-c3ncccn3)c3ccccc23)ccc1F]

### Example 194: trans-4-(2-(4-Fluoro-3-methoxyphenyl)cyclopropyl)-1-(pyrimidin-2-yl)isoquinoline (single enantiomer II)

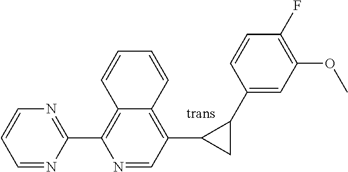 [SMILES: COc1cc(C2CC2c2cnc(-c3ncccn3)c3ccccc23)ccc1F]

A mixture of trans-4-(2-(4-fluoro-3-methoxyphenyl)cyclopropyl)-1-(pyrimidin-2-yl)isoquinoline (130 mg) was separated by SFC on a LUX® Amylose-2 column using liquid CO₂ and 30 mM Methanolic ammonia in EtOH (50:50) to give trans-4-(2-(4-fluoro-3-methoxyphenyl)cyclopropyl)-1-(pyrimidin-2-yl)isoquinoline (single enantiomer I) as an off-white solid (faster eluting enantiomer, 15 mg, 12% yield, m/z: 372 [M+H]⁺ observed) and trans-4-(2-(4-fluoro-3-methoxyphenyl)cyclopropyl)-1-(pyrimidin-2-yl)isoquinoline (single enantiomer II) as an off-white solid (slower eluting enantiomer, 20 mg, 15% yield, m/z: 372 [M+H]⁺ observed).

### Example 193: trans-4-(2-(4-Fluoro-3-methoxyphenyl)cyclopropyl)-1-(pyrimidin-2-yl)isoquinoline (single enantiomer I)

m/z: 372 [M+H]⁺ observed. ¹H NMR (400 MHz, DMSO-d₆): δ 9.05-9.04 (m, 2H), 8.51 (s, 1H), 8.23 (d, 1H), 8.07 (d, 1H), 7.87-7.83 (m, 1H), 7.69-7.64 (m, 2H), 7.19-7.11 (m, 2H), 6.93-6.89 (m, 1H), 3.88 (s, 3H), 2.82-2.77 (m, 1H), 2.35-2.31 (m, 1H), 1.78-1.74 (m, 1H), 1.65-1.62 (m, 1H).

### Example 194: trans-4-(2-(4-Fluoro-3-methoxyphenyl)cyclopropyl)-1-(pyrimidin-2-yl)isoquinoline (single enantiomer II)

m/z: 372 [M+H]⁺ observed. ¹H NMR (400 MHz, DMSO-d₆): δ 9.05-9.04 (m, 2H), 8.51 (s, 1H), 8.23 (d, 1H), 8.07 (d, 1H), 7.87-7.83 (m, 1H), 7.69-7.64 (m, 2H), 7.19-7.11 (m, 2H), 6.93-6.89 (m, 1H), 3.88 (s, 3H), 2.82-2.77 (m, 1H), 2.35-2.31 (m, 1H), 1.78-1.74 (m, 1H), 1.65-1.62 (m, 1H).

### Example 195: trans-5-(2-(4-Fluoro-3-(piperidin-1-yl)phenyl)cyclopropyl)-2,2′-bipyrimidine (single enantiomer I)

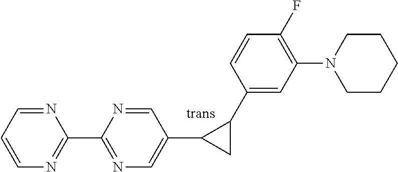 [SMILES: Fc1ccc(C2CC2c2cnc(-c3ncccn3)nc2)cc1N1CCCCC1]

### Example 196: trans-5-(2-(4-Fluoro-3-(piperidin-1-yl)phenyl)cyclopropyl)-2,2′-bipyrimidine (single enantiomer II)

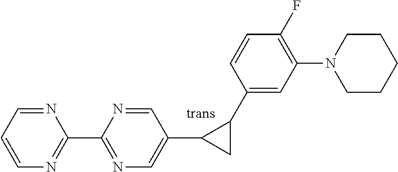 [SMILES: Fc1ccc(C2CC2c2cnc(-c3ncccn3)nc2)cc1N1CCCCC1]

A mixture of trans-5-(2-(4-fluoro-3-(piperidin-1-yl)phenyl)cyclopropyl)-2,2′-bipyrimidine (220 mg) was separated by SFC on a CHIRALPAK® OD-H column using liquid CO₂ and MeOH (50:50) to give trans-5-(2-(4-fluoro-3-(piperidin-1-yl)phenyl)cyclopropyl)-2,2′-bipyrimidine (single enantiomer I) as a pale orange solid (faster eluting enantiomer, 64 mg, 29% yield, m/z: 376 [M+H]⁺ observed) and trans-5-(2-(4-fluoro-3-(piperidin-1-yl)phenyl)cyclopropyl)-2,2′-bipyrimidine (single enantiomer II) as a pale orange solid (slower eluting enantiomer, 60 mg, 27% yield, m/z: 376 [M+H]⁺ observed).

### Example 195: trans-5-(2-(4-Fluoro-3-(piperidin-1-yl)phenyl)cyclopropyl)-2,2′-bipyrimidine (single enantiomer I)

m/z: 376 [M+H]⁺ observed. ¹H NMR (400 MHz, DMSO-d₆): δ 8.99 (d, 2H), 8.83 (s, 2H), 7.62 (t, 1H), 7.05-7.00 (m, 1H), 6.88-6.85 (m, 1H), 6.79-6.75 (m, 1H), 2.98-2.95 (m, 4H), 2.50-2.46 (m, 1H), 2.36-2.32 (m, 1H), 1.71-1.59 (m, 6H), 1.56-1.55 (m, 2H).

### Example 196: trans-5-(2-(4-Fluoro-3-(piperidin-1-yl)phenyl)cyclopropyl)-2,2′-bipyrimidine (single enantiomer II)

m/z: 376 [M+H]⁺ observed. ¹H NMR (400 MHz, DMSO-d₆): δ 8.99 (d, 2H), 8.83 (s, 2H), 7.62 (t, 1H), 7.05-7.00 (m, 1H), 6.88-6.85 (m, 1H), 6.79-6.75 (m, 1H), 2.98-2.95 (m, 4H), 2.50-2.46 (m, 1H), 2.36-2.32 (m, 1H), 1.71-1.59 (m, 6H), 1.56-1.55 (m, 2H).

### Example 197: trans-1-(5-(2-([2,2′-Bipyrimidin]-5-yl)cyclopropyl)-2-fluorophenyl)pyrrolidin-2-one (single enantiomer I)

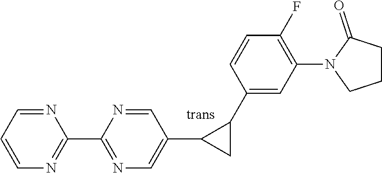 [SMILES: O=C1CCCN1c1cc(C2CC2c2cnc(-c3ncccn3)nc2)ccc1F]

### Example 198: trans-1-(5-(2-([2,2′-Bipyrimidin]-5-yl)cyclopropyl)-2-fluorophenyl)pyrrolidin-2-one (single enantiomer II)

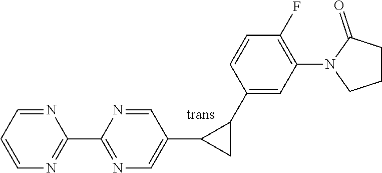 [SMILES: O=C1CCCN1c1cc(C2CC2c2cnc(-c3ncccn3)nc2)ccc1F]

A mixture of trans-1-(5-(2-([2,2′-bipyrimidin]-5-yl)cyclopropyl)-2-fluorophenyl)pyrrolidin-2-one (200 mg) was separated by SFC on a CHIRALPAK® OD-H column using liquid CO₂ and MeOH (45:55) to give trans-1-(5-(2-([2,2′-bipyrimidin]-5-yl)cyclopropyl)-2-fluorophenyl)pyrrolidin-2-one (single enantiomer I) as a brown solid (faster eluting enantiomer, 40 mg, 20% yield, m/z: 376 [M+H]⁺ observed) and trans-1-(5-(2-([2,2′-bipyrimidin]-5-yl)cyclopropyl)-2-fluorophenyl)pyrrolidin-2-one (single enantiomer II) as a brown solid (slower eluting enantiomer, 40 mg, 20% yield, m/z: 376 [M+H]⁺ observed).

### Example 197: trans-1-(5-(2-([2,2′-Bipyrimidin]-5-yl)cyclopropyl)-2-fluorophenyl)pyrrolidin-2-one (single enantiomer I)

m/z: 376 [M+H]⁺ observed. ¹H NMR (400 MHz, DMSO-d₆): δ 8.99 (d, 2H), 8.85 (s, 2H), 7.62 (t, 1H), 7.32-7.17 (m, 3H), 3.76 (t, 2H), 2.55-2.50 (m, 1H), 2.42 (t, 2H), 2.37-2.34 (m, 1H), 2.14-2.10 (m, 2H), 1.73-1.65 (m, 2H).

### Example 198: trans-1-(5-(2-([2,2′-Bipyrimidin]-5-yl)cyclopropyl)-2-fluorophenyl)pyrrolidin-2-one (single enantiomer II)

m/z: 376 [M+H]⁺ observed. ¹H NMR (400 MHz, DMSO-d₆): δ 8.99 (d, 2H), 8.85 (s, 2H), 7.62 (t, 1H), 7.32-7.17 (m, 3H), 3.76 (t, 2H), 2.55-2.50 (m, 1H), 2.42 (t, 2H), 2.37-2.34 (m, 1H), 2.14-2.10 (m, 2H), 1.73-1.65 (m, 2H).

### Example 199: trans-5-(2-(4-Chloro-3-(pyrrolidin-1-yl)phenyl)cyclopropyl)-2,2′-bipyrimidine (single enantiomer I)

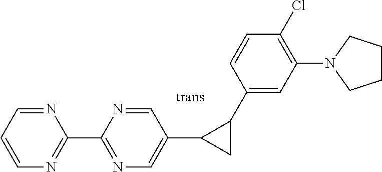 [SMILES: Clc1ccc(C2CC2c2cnc(-c3ncccn3)nc2)cc1N1CCCC1]

### Example 200: trans-5-(2-(4-Chloro-3-(pyrrolidin-1-yl)phenyl)cyclopropyl)-2,2′-bipyrimidine (single enantiomer II)

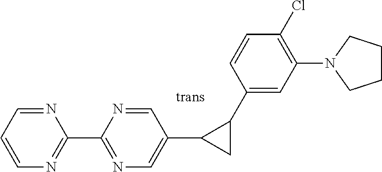 [SMILES: Clc1ccc(C2CC2c2cnc(-c3ncccn3)nc2)cc1N1CCCC1]

A mixture of trans-5-(2-(4-chloro-3-(pyrrolidin-1-yl)phenyl)cyclopropyl)-2,2′-bipyrimidine (200 mg) was separated by SFC on a CHIRALPAK® OD-H column using liquid CO₂ and MeOH (45:55) to give trans-5-(2-(4-chloro-3-(pyrrolidin-1-yl)phenyl)cyclopropyl)-2,2′-bipyrimidine (single enantiomer I) as a pale grey solid (faster eluting enantiomer, 70 mg, 35% yield, m/z: 378 [M+H]⁺ observed) and trans-5-(2-(4-chloro-3-(pyrrolidin-1-yl)phenyl)cyclopropyl)-2,2′-bipyrimidine (single enantiomer II) as a pale grey solid (slower eluting enantiomer, 62 mg, 31% yield, m/z: 378 [M+H]⁺ observed).

### Example 199: trans-5-(2-(4-Chloro-3-(pyrrolidin-1-yl)phenyl)cyclopropyl)-2,2′-bipyrimidine (single enantiomer I)

m/z: 378 [M+H]⁺ observed. ¹H NMR (400 MHz, DMSO-d₆): δ 8.99 (d, 2H), 8.83 (s, 2H), 7.62 (t, 1H), 7.21 (d, 1H), 6.81 (s, 1H), 6.65-6.63 (m, 1H), 3.31(m, 4H), 2.49-2.46 (m, 1H), 2.36-2.32 (m, 1H), 1.89-1.85 (m, 4H), 1.72-1.70 (m, 1H), 1.64-1.62 (m, 1H).

### Example 200: trans-5-(2-(4-Chloro-3-(pyrrolidin-1-yl)phenyl)cyclopropyl)-2,2′-bipyrimidine (single enantiomer II)

m/z: 378 [M+H]⁺ observed. ¹H NMR (400 MHz, DMSO-d₆): δ 8.99 (d, 2H), 8.85 (s, 2H), 7.62 (t, 1H), 7.32-7.17 (m, 3H), 3.76 (t, 2H), 2.55-2.50 (m, 1H), 2.42 (t, 2H), 2.37-2.34 (m, 1H), 2.14-2.10 (m, 2H), 1.73-1.65 (m, 2H).

### Example 201: trans-5-02-([2,2′-Bipyrimidin]-5-yl)cyclopropyl)-2-chloro-N-methylbenzamide (single enantiomer I)

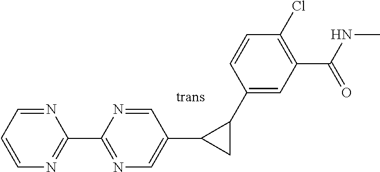 [SMILES: CNC(=O)c1cc(C2CC2c2cnc(-c3ncccn3)nc2)ccc1Cl]

### Example 202: trans-5-02-([2,2′-Bipyrimidin]-5-yl)cyclopropyl)-2-chloro-N-methylbenzamide (single enantiomer II)

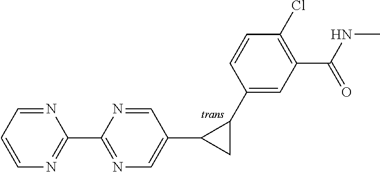 [SMILES: CNC(=O)c1cc(C2CC2c2cnc(-c3ncccn3)nc2)ccc1Cl]

### Methyl 5-bromo-2-chlorobenzoate

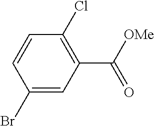 [SMILES: COC(=O)c1cc(Br)ccc1Cl]

To a mixture of 5-bromo-2-chloro-benzoic acid (40 g, 170 mmol) and MeOH (1.2 L, 29.7 mol) was added concentrated H2SO₄ (9.1 mL, 170 mmol) in one portion and the reaction was stirred for 16 hr at 60° C. The mixture (combined with another batch at same scale) was concentrated directly. Then saturated aqueous sodium bicarbonate solution was added to adjust the pH to 3 and the mixture was extracted with EtOAc (3×300 mL). The combined organic phase was washed with saturated aqueous brine solution (300 mL), dried over Na₂SO₄, filtered, and concentrated under vacuum. The residue was purified by normal phase SiO₂ chromatography (petroleum ether) to afford methyl 5-bromo-2-chlorobenzoate as a white solid (72 g, 85% yield). ¹H NMR (400 MHz, CDCl₃): δ 7.97 (d, J=1.6 Hz, 1H), 7.55-7.53 (m, 1H), 7.33 (d, J=8.4 Hz, 1H), 3.94 (s, 3H).

### Methyl 2-chloro-5-vinylbenzoate

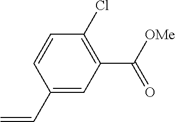

To a mixture of methyl 5-bromo-2-chlorobenzoate (35 g, 140 mmol) and potassium vinyltrifluoroborate (18.8 g, 140 mmol) in THF (630 mL) and H₂O (70 mL) was added Pd(dppf)Cl₂. CH₂Cl₂ (11.5 g, 14 mmol), followed by Cs₂CO₃ (137 g, 421 mmol) under N₂. The reaction mixture was stirred at 80° C. for 3.5 hr. The mixture was filtered and washed with EtOAc (500 mL). Water (500 mL) was added to the filtrate and extracted with EtOAc (3×300 mL). The combined organic phase was washed with saturated aqueous brine solution (500 mL), dried over Na₂SO₄, filtered, and concentrated under vacuum. The residue was purified by normal phase SiO₂ chromatography (petroleum ether) to afford methyl 2-chloro-5-vinylbenzoate as light-yellow oil (23 g, 82% yield). ¹H NMR (400 MHz, CDCl₃): δ 7.84 (s, 1H), 7.46-7.39 (m, 2H), 6.68 (dd, J=17.6, 10.8 Hz, 1H), 5.79 (d, J=17.6 Hz, 1H), 5.34 (d, J=10.8 Hz, 1H), 3.95 (s, 3H).

### (E)-Methyl 2-chloro-5-(2-(2-chloropyrimidin-5-yl)vinyl)benzoate

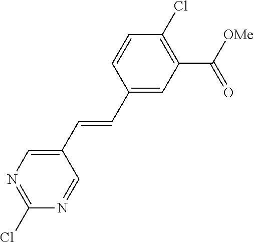 [SMILES: COC(=O)c1cc(/C=C/c2cnc(Cl)nc2)ccc1Cl]

To a solution of methyl 2-chloro-5-vinylbenzoate (23 g, 117 mmol) in acetonitrile (460 mL) was added 5-bromo-2-chloropyrimidine (45.3 g, 234 mmol) and DIPEA (61 mL, 351 mmol) under N2, followed by Pd(OAc)₂ (5.25 g, 23.4 mmol). The reaction mixture was heated at 100° C. for 16 h under N₂. The mixture was filtered and washed with EtOAc (400 mL). Water (500 mL) was added to the filtrate, the layers separated and the aqueous phase was extracted with EtOAc (3×400 mL). The combined organic phase was washed with saturated aqueous brine solution (500 mL), dried over Na₂SO₄, filtered, and concentrated under vacuum. The residue was purified by normal phase SiO₂ chromatography (0-25% EtOAc/petroleum ether), followed by reverse phase HPLC to afford (E)-methyl 2-chloro-5-(2-(2-chloropyrimidin-5-yl)vinyl)benzoate as a light-yellow solid (13 g, 36% yield, m/z: 309 [M+H]⁺ observed). trans-Methyl 2-chloro-5-(2-(2-chloropyrimidin-5-yl)cyclopropyl)benzoate:

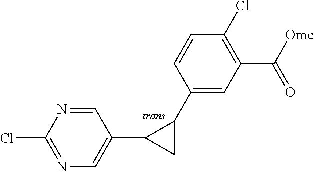

To a solution of (E)-methyl 2-chloro-5-(2-(2-chloropyrimidin-5-yl)vinyl)benzoate (1 g, 3.23 mmol) in THF (16 mL) was added Pd(OAc)₂ (72.6 mg, 0.32 mmol), then to the mixture was added dropwise diazomethane solution (0.5 M in Et₂O, 259 mL, 130 mmol) at −20° C. The mixture was stirred at −20° C. for 1 h. The reaction mixture (combined with another 3 batches at same scale) was filtered and the filtrate was concentrated under vacuum. The residue was purified by normal phase SiO₂ chromatography (9-25% EtOAc/petroleum ether) to afford trans-methyl 2-chloro-5-(2-(2-chloropyrimidin-5-yl)cyclopropyl)benzoate as a yellow solid (1.46 g, 17% yield, m/z: 323 [M+H]⁺ observed).

### trans-Methyl 5-(2-([2,2′-bipyrimidin]-5-yl)cyclopropyl)-2-chlorobenzoate

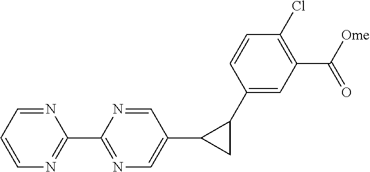

To a solution of trans-methyl 2-chloro-5-(2-(2-chloropyrimidin-5-yl)cyclopropyl)benzoate (1.46 g, 4.52 mmol) and 2-(tributylstannyl)pyrimidine (1.6 mL, 4.97 mmol) in DMF (30 mL) was added tetraethylammonium chloride (0.75 g, 4.5 mmol) and potassium carbonate (1.25 g, 9.04 mmol) under N₂ followed by Pd(PPh₃)₂Cl₂ (0.32 g, 0.45 mmol). The reaction mixture was stirred at 110° C. for 6 h. The reaction mixture was purified directly by normal phase SiO₂ chromatography (0-100% EtOAc/petroleum ether, followed by 0-20% MeOH/EtOAc), followed by reverse phase HPLC to afford trans-methyl 5-(2-([2,2′-bipyrimidin]-5-yl)cyclopropyl)-2-chlorobenzoate as a yellow solid (75 mg, 5% yield, m/z: 367 [M+H]⁺ observed).

### trans-5-(2-([2,2′-Bipyrimidin]-5-yl)cyclopropyl)-2-chloro-N-methylbenzamide

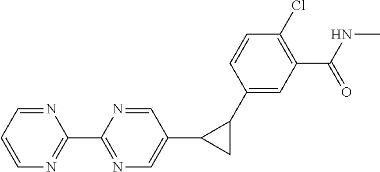 [SMILES: CNC(=O)c1cc(C2CC2c2cnc(-c3ncccn3)nc2)ccc1Cl]

A mixture of trans-methyl 5-(2-([2,2′-bipyrimidin]-5-yl)cyclopropyl)-2-chlorobenzoate (70 mg, 0.19 mmol) and MeNH₂ (30 wt. % in EtOH, 1.8 mL, 19 mmol) was stirred at rt under N₂ atmosphere for 16 hr. The mixture was concentrated in vacuum. The residue was purified by reverse phase HPLC to afford trans-5-(2-([2,2′-bipyrimidin]-5-yl)cyclopropyl)-2-chloro-N-methylbenzamide as a white solid (50 mg, 72% yield, m/z: 366 [M+H]⁺ observed). A mixture of enantiomers of trans-5-((2-([2,2′-bipyrimidin]-5-yl)cyclopropyl)-2-chloro-N-methylbenzamide (50 mg) was separated by SFC (supercritical fluid chromatography) on a CHIRALCEL® OD column using liquid CO₂ and MeOH [0.1% aqueous NH₃ as modifier] (50:50) to give trans-5-((2-([2,2′-bipyrimidin]-5-yl)cyclopropyl)-2-chloro-N-methylbenzamide (single enantiomer I) as a white solid (faster eluting enantiomer, 11 mg, 22%, m/z: 366 [M+H]⁺ observed), and trans-5-(2-([2,2′-bipyrimidin]-5-yl)cyclopropyl)-2-chloro-N-methylbenzamide (single enantiomer II) as a white solid (slower eluting enantiomer, 13 mg, 26%, m/z: 366 [M+H]⁺ observed).

### Example 201: trans-5-(2-([2,2′-Bipyrimidin]-5-yl)cyclopropyl)-2-chloro-N-methylbenzamide (single enantiomer I)

m/z: 366 [M+H]⁺ observed. ¹H NMR (400 MHz, DMSO-d₆): δ 8.99 (d, J=4.8 Hz, 2 H), 8.85 (s, 2 H), 8.33-8.31 (m, 1 H), 7.62 (t, J=4.8 Hz, 1 H), 7.41 (d, J=8.4 Hz, 1 H), 7.31-7.29 (m, 2 H), 2.75 (d, J=4.4 Hz, 3 H), 2.61-2.57 (m, 1 H), 2.41-2.38 (m, 1 H), 1.79-1.76 (m, 1 H), 1.68-1.66 (m, 1 H).

### Example 202: trans-5-(2-([2,2′-Bipyrimidin]-5-yl)cyclopropyl)-2-chloro-N-methylbenzamide (single enantiomer II)

m/z: 366 [M+H]⁺ observed. ¹H NMR (400 MHz, DMSO-d₆): δ 8.99 (d, J=4.8 Hz, 2 H), 8.85 (s, 2 H), 8.33-8.31 (m, 1 H), 7.62 (t, J=4.8 Hz, 1 H), 7.41 (d, J=8.4 Hz, 1 H), 7.31-7.29 (m, 2 H), 2.75 (d, J=4.4 Hz, 3 H), 2.61-2.57 (m, 1 H), 2.41-2.38 (m, 1 H), 1.79-1.76 (m, 1 H), 1.68-1.66 (m, 1 H).

The following examples were prepared in a similar manner as trans-5-(2-([2,2′-bipyrimidin]-5-yl)cyclopropyl)-2-chloro-N-methylbenzamide from trans-methyl 5-(2-([2,2′-bipyrimidin]-5-yl)cyclopropyl)-2-chlorobenzoate and dimethyl amine:

### Example 203: trans-5-(2-([2,2′-Bipyrimidin]-5-yl)cyclopropyl)-2-chloro-N,N-dimethylbenzamide (single enantiomer I)

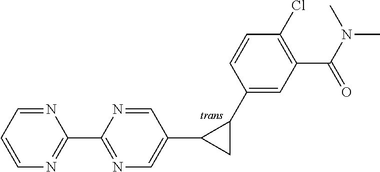 [SMILES: CN(C)C(=O)c1cc(C2CC2c2cnc(-c3ncccn3)nc2)ccc1Cl]

### Example 204: trans-5-(2-([2,2′-Bipyrimidin]-5-yl)cyclopropyl)-2-chloro-N,N-dimethylbenzamide (single enantiomer II)

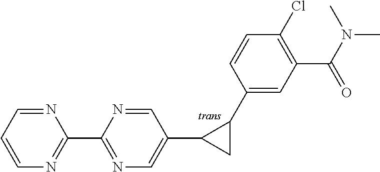 [SMILES: CN(C)C(=O)c1cc(C2CC2c2cnc(-c3ncccn3)nc2)ccc1Cl]

A mixture of enantiomers of trans-5-(2-([2,2′-bipyrimidin]-5-yl)cyclopropyl)-2-chloro-N,N-dimethylbenzamide (27 mg) was separated by SFC on a CHIRALCEL® OD column using liquid CO₂ and MeOH [0.1% aqueous NH₃ modifier] (40:60) to give trans-5-(2-([2,2′-bipyrimidin]-5-yl)cyclopropyl)-2-chloro-N,N-dimethylbenzamide (single enantiomer I) as a yellow solid (faster eluting enantiomer, 5 mg, 19% yield, m/z: 380 [M+H]⁺ observed), and trans-5-(2-([2,2′-bipyrimidin]-5-yl)cyclopropyl)-2-chloro-N,N-dimethyl benzamide (single enantiomer II) as a yellow solid (slower eluting enantiomer, 5 mg, 19% yield, m/z: 380 [M+H]⁺ observed).

### Example 203: trans-5-(2-([2,2′-Bipyrimidin]-5-yl)cyclopropyl)-2-chloro-N,N-dimethylbenzamide (single enantiomer I)

m/z: 380 [M+H]⁺ observed. ¹H NMR (400 MHz, DMSO-d₆): δ 8.99 (d, J=5.2 Hz, 2 H), 8.85 (s, 2 H), 7.62 (t, J=4.8 Hz, 1 H), 7.44 (d, J=8.4 Hz, 1 H), 7.30 (d, J=8 Hz, 1 H), 7.23 (s, 1H), 3.00 (s, 3 H), 2.79 (s, 3 H), 2.61-2.58 (m, 1 H), 2.43-2.41 (m, 1 H), 1.79-1.66 (m, 2 H).

### Example 204: trans-5-(2-([2,2′-Bipyrimidin]-5-yl)cyclopropyl)-2-chloro-N,N-dimethylbenzamide (single enantiomer II)

m/z: 380 [M+H]⁺ observed. ¹H NMR (400 MHz, DMSO-d₆): δ 8.99 (d, J=5.2 Hz, 2 H), 8.85 (s, 2 H), 7.62 (t, J=4.8 Hz, 1 H), 7.44 (d, J=8.4 Hz, 1 H), 7.30 (d, J=8 Hz, 1 H), 7.23 (s, 1H), 3.00 (s, 3 H), 2.79 (s, 3 H), 2.61-2.58 (m, 1 H), 2.43-2.41 (m, 1 H), 1.79-1.66 (m, 2 H).

### Example 205: trans-N-(5-(2-([2,2′Bipyrimidin]-5-yl)cyclopropyl)-2-chlorophenyl)acetamide (single enantiomer I)

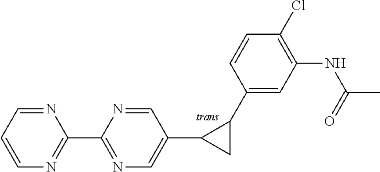 [SMILES: CC(=O)Nc1cc(C2CC2c2cnc(-c3ncccn3)nc2)ccc1Cl]

### Example 206: trans-N-(5-(2-([2,2′-Bipyrimidin]-5-yl)cyclopropyl)-2-chlorophenyl)acetamide (single enantiomer II)

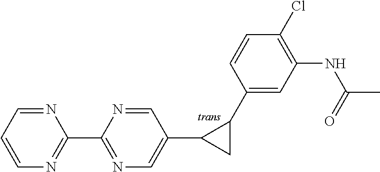 [SMILES: CC(=O)Nc1cc(C2CC2c2cnc(-c3ncccn3)nc2)ccc1Cl]

### trans-2-Chloro-5-(2-(4-chloro-3-nitrophenyl)cyclopropyl)pyrimidine

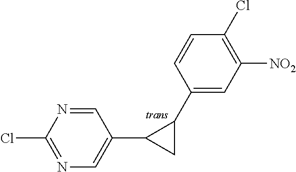 [SMILES: O=[N+]([O-])c1cc(C2CC2c2cnc(Cl)nc2)ccc1Cl]

To a mixture of 4-bromo-1-chloro-2-nitrobenzene (0.5 mL, 4.2 mmol) and trans-2-chloro-5-(2-(4,4,5,5-tetramethyl-1,3,2-dioxaborolan-2-yl)cyclopropyl)pyrimidine (1.3 g, 4.7 mmol) in THF (8 mL) and H₂O (2 mL) was added Cs₂CO₃ (2.76 g, 8.46 mmol), followed by Pd(dppf)Cl₂. CH₂Cl₂ (345 mg, 0.42 mmol) in one portion under N₂. The mixture was stirred at 80° C. for 16 h. The reaction mixture was cooled to room temperature and H₂O (50 mL) was added. The mixture was extracted with EtOAc (2×30 mL). The combined organic phase was washed with saturated aqueous brine solution (50 mL), dried over Na₂SO₄, filtered, and concentrated under vacuum. The residue was purified by normal phase SiO₂ chromatography (0-15% EtOAc/petroleum ether) to afford trans-2-chloro-5-(2-(4-chloro-3-nitrophenyl)cyclopropyl)pyrimidine as a yellow solid (0.80 g, 62% yield, m/z: 310 [M+H]⁺ observed).

### trans-5-(2-(4-Chloro-3-nitrophenyl)cyclopropyl)-2,2′-bipyrimidine

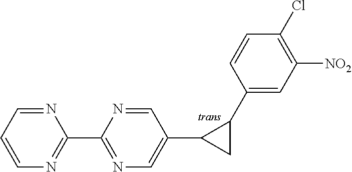 [SMILES: O=[N+]([O-])c1cc(C2CC2c2cnc(-c3ncccn3)nc2)ccc1Cl]

To a mixture of trans-2-chloro-5-(2-(4-chloro-3-nitrophenyl)cyclopropyl)pyrimidine (600 mg, 1.93 mmol, 85% purity) and 2-(tributylstannyl)pyrimidine (0.7 mL, 2.13 mmol) in DMF (5 mL) was added K₂CO₃ (294 mg, 2.13 mmol) and tetraethylammonium chloride (321 mg, 1.93 mmol) and Pd(dppf)Cl₂ (142 mg, 0.19 mmol) in one portion under N₂. The mixture was stirred at 110° C. for 12 hours. The mixture was cooled to rt and purified directly without further work up. The mixture was purified by normal phase SiO₂ chromatography (0-15% MeOH/CH₂Cl₂) which was further purified by reverse phase HPLC to afford trans-5-(2-(4-chloro-3-nitrophenyl)cyclopropyl)-2,2′-bipyrimidine as a white solid (55 mg, 8% yield, m/z: 354 [M+H]⁺ observed).

### trans-5-(2-([2,2′-Bipyrimidin]-5-yl)cyclopropyl)-2-chloroandine

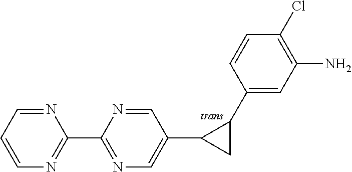 [SMILES: Nc1cc(C2CC2c2cnc(-c3ncccn3)nc2)ccc1Cl]

To a mixture of trans-5-(2-(4-chloro-3-nitrophenyl)cyclopropyl)-2,2′-bipyrimidine (55 mg, 0.16 mmol) in saturated aqueous NH₄Cl solution (1 mL) and EtOH (1 mL) was added Fe (87 mg, 1.55 mmol) in one portion. The mixture was stirred at rt for 1 hour. The mixture was filtered and the filtrate was concentrated in reduced pressure to give trans-5-(2-([2,2′-bipyrimidin]-5-yl)cyclopropyl)-2-chloroaniline as a yellow solid (40 mg, m/z: 324 [M+H]⁺ observed), which was used in the next step without further purification.

### trans-N-(5-(2-([2,2′-Bipyrimidin]-5-yl)cyclopropyl)-2-chlorophenyl)acetamide

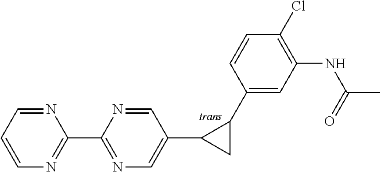 [SMILES: CC(=O)Nc1cc(C2CC2c2cnc(-c3ncccn3)nc2)ccc1Cl]

A mixture of trans-5-(2-([2,2′-bipyrimidin]-5-yl)cyclopropyl)-2-chloroaniline (40 mg, 0.12 mmol) and acetic anhydride (5 mL, 53.4 mmol) was stirred at rt for 0.5 hour. The mixture was concentrated in vacuum. The residue was purified by reverse phase HPLC to afford trans-N-(5-(2-([2,2′-bipyrimidin]-5-yl)cyclopropyl)-2-chlorophenyl)acetamide as a yellow solid (15 mg, 33% yield, m/z: 366 [M+H]⁺ observed).

A mixture of enantiomers of trans-N-(5-(2-([2,2′-bipyrimidin]-5-yl)cyclopropyl)-2-chlorophenyl)acetamide (35 mg) was separated by SFC (supercritical fluid chromatography) on a CHIRALPAK® AD column using liquid CO₂ and IPA [0.1% aqueous NH₃ as modifier] (45:55) to give trans-N-(5-(2-([2,2′-bipyrimidin]-5-yl)cyclopropyl)-2-chlorophenyl)acetamide (single enantiomer I) as a white solid (faster eluting enantiomer, 3 mg, 11% yield, m/z: 366 [M+H]⁺ observed), and trans-N-(5-(2-([2,2′-bipyrimidin]-5-yl)cyclopropyl)-2-chlorophenyl)acetamide (single enantiomer II) as a yellow solid (slower eluting enantiomer, 5 mg, 15% yield, m/z: 366 [M+H]⁺ observed).

### Example 205: trans-N-(5-(2-([2,2′-Bipyrimidin]-5-yl)cyclopropyl)-2-chlorophenyl)acetamide (single enantiomer I)

m/z: 366 [M+H]⁺ observed. ¹H NMR (400 MHz, Me0D): 6 9.02 (d, J=4.8 Hz, 2H), 8.85 (s, 2H), 7.63 (t, J=4.8 Hz, 2H), 7.38 (d, J=8.4 Hz, 1H), 7.08 (d, J=7.6 Hz, 1H), 2.54-2.49 (m, 1H), 2.41-2.36 (m, 1H), 2.09 (s, 3H), 1.76-1.66 (m, 2H).

### Example 206: trans-N-(5-(2-([2,2′-Bipyrimidin]-5-yl)cyclopropyl)-2-chlorophenyl)acetamide (single enantiomer II)

m/z: 366 [M+H]⁺ observed. ¹H NMR (400 MHz, Me0D): 6 9.02 (d, J=4.8 Hz, 2H), 8.85 (s, 2H), 7.63 (t, J=4.8 Hz, 2H), 7.38 (d, J=8.4 Hz, 1H), 7.08 (d, J=7.6 Hz, 1H), 2.54-2.49 (m, 1H), 2.41-2.36 (m, 1H), 2.09 (s, 3H), 1.76-1.66 (m, 2H).

### Example 207: trans-5-(2-([2,2′-Bipyrimidin]-5-yl)cyclopropyl)-N-cyclopentyl-2,3-difluoroaniline (single enantiomer I)

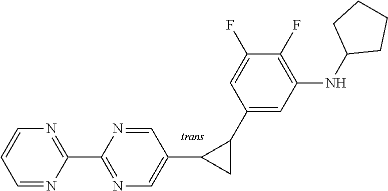 [SMILES: Fc1cc(C2CC2c2cnc(-c3ncccn3)nc2)cc(NC2CCCC2)c1F]

### Example 208: trans-5-(2-([2,2′Bipyrimidin]-5-yl)cyclopropyl)-N-cyclopentyl-2,3-difluoroaniline (single enantiomer II):

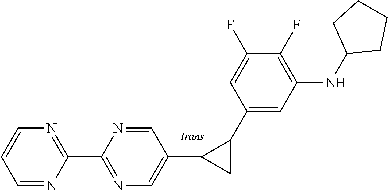 [SMILES: Fc1cc(C2CC2c2cnc(-c3ncccn3)nc2)cc(NC2CCCC2)c1F]

### trans-5-(2-([2,2′-Bipyrimidin]-5-yl)cyclopropyl)-N-cyclopentyl-2,3-difluoroaniline

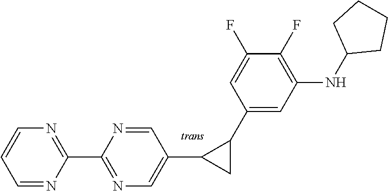 [SMILES: Fc1cc(C2CC2c2cnc(-c3ncccn3)nc2)cc(NC2CCCC2)c1F]

To a mixture of trans-5-(2-(3,4,5-trifluorophenyl)cyclopropyl)-2,2′-bipyrimidine (200 mg, 0.6 mmol) in DMSO (1 mL) was added cyclopentylamine (150 mg, 1.83 mmol) in one portion under N₂. The mixture was stirred at 180° C. for 2 hours with microwave irradiation. The mixture was combined with another two batches at same scale. The mixture was purified directly by normal phase SiO₂ chromatography (0-9% MeOH/CH₂Cl₂) to give trans-5-(2-([2,2′-bipyrimidin]-5-yl)cyclopropyl)-N-cyclopentyl-2,3-difluoroaniline as a white solid (85 mg, 36% yield, m/z: 394 [M+H]⁺ observed).

A mixture of enantiomers of trans-5-(2-([2,2′-bipyrimidin]-5-yl)cyclopropyl)-N-cyclopentyl-2,3-difluoroaniline (85 mg) was separated by SFC (supercritical fluid chromatography) on a CHIRALCEL® OD column using liquid CO₂ and EtOH [0.1% aqueous NH₃ modifier] (45:55) to give trans-5-(2-([2,2′-bipyrimidin]-5-yl)cyclopropyl)-N-cyclopentyl-2,3-difluoroaniline (single enantiomer I) as a white solid (faster eluting enantiomer, 32 mg, 36% yield, m/z: 394 [M+H]⁺ observed) and trans-5-(2-([2,2′-bipyrimidin]-5-yl)cyclopropyl)-N-cyclopentyl-2,3-difluoroaniline (single enantiomer II) as a white solid (slower eluting enantiomer, 27 mg, 30% yield, m/z: 394 [M+H]⁺ observed).

### Example 207: trans-5-(2-([2,2′-Bipyrimidin]-5-yl)cyclopropyl)-N-cyclopentyl-2,3-difluoroaniline (single enantiomer I)

m/z: 394 [M+H]⁺ observed. ¹H NMR (400 MHz, DMSO-d₆): δ 8.99 (d, J=4.8 Hz, 2 H), 8.83 (s, 2 H), 7.62 (t, J=4.8 Hz, 1 H), 6.45 (d, J=6.8 Hz, 1 H), 6.38-6.34 (m, 1H), 5.55 (d, J=6 Hz, 1 H), 3.82-3.77 (m, 1 H), 2.45-2.43 (m, 1 H), 2.33-2.32 (m, 1H), 1.95-1.94 (m, 2 H), 1.69-1.66 (m, 3H), 1.53-1.51 (m, 5H).

### Example 208: trans-5-(2-([2,2′-Bipyrimidin]-5-yl)cyclopropyl)-N-cyclopentyl-2,3-difluoroaniline (single enantiomer II)

m/z: 394 [M+H]⁺ observed. ¹H NMR (400 MHz, DMSO-d₆): δ 8.99 (d, J=4.8 Hz, 2 H), 8.83 (s, 2 H), 7.62 (t, J=4.8 Hz, 1 H), 6.45 (d, J=6.8 Hz, 1 H), 6.38-6.34 (m, 1H), 5.55 (d, J=6 Hz, 1 H), 3.82-3.77 (m, 1 H), 2.45-2.43 (m, 1 H), 2.33-2.32 (m, 1H), 1.95-1.94 (m, 2 H), 1.69-1.66 (m, 3H), 1.53-1.51 (m, 5H).

The following examples were prepared in a similar manner as trans-5-(2-([2,2′-bipyrimidin]-5-yl)cyclopropyl)-N-cyclopentyl-2,3-difluoroaniline from trans-5-(2-(3,4,5-trifluorophenyl)cyclopropyl)-2,2′-bipyrimidine and an appropriate amine.

### Example 209: trans-6-(5-(2-([2,2′-Bipyrimidin]-5-yl)cyclopropyl)-2,3-difluorophenyl)-2-oxa-6-azaspiro[3.3]heptane

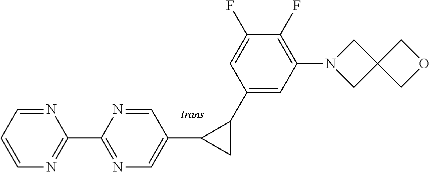 [SMILES: Fc1cc(C2CC2c2cnc(-c3ncccn3)nc2)cc(N2CC3(COC3)C2)c1F]

m/z: 408 [M+H]⁺ observed. ¹H NMR (400 MHz, CDCl₃): δ 9.12-8.94 (m, 2H), 8.75 (s, 2H), 7.42 (tdd, J=4.8, 1.6, 0.5 Hz, 1H), 6.31 (ddd, J=10.9, 6.5, 2.2 Hz, 1H), 6.02 (dt, J=7.3, 1.9 Hz, 1H), 4.83 (s, 4H), 4.15 (d, J=2.1 Hz, 4H), 2.23 (ddd, J=8.7, 6.4, 4.5 Hz, 1H), 2.20-2.11 (m, 1H), 1.60-1.54 (m, 2H).

### Example 210: trans-5-(2-(3,4-Difluoro-5-(3-isopropoxyazetidin-1-yl)phenyl)cyclopropyl)-2,2′-bipyrimidine

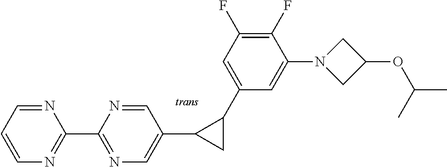 [SMILES: CC(C)OC1CN(c2cc(C3CC3c3cnc(-c4ncccn4)nc3)cc(F)c2F)C1]

m/z: 424 [M+H]⁺ observed. ¹H NMR (400 MHz, CDCl₃): δ 9.01 (d, J=4.9 Hz, 2H), 8.75 (s, 2H), 7.42 (t, J=4.8 Hz, 1H), 6.31 (ddd, J=10.9, 6.2, 2.2 Hz, 1H), 6.04 (d, J=7.3 Hz, 1H), 4.46 (q, J=5.8 Hz, 1H), 4.33-4.20 (m, 2H), 3.86-3.74 (m, 2H), 3.65 (p, J=6.1 Hz, 1H), 2.29-2.19 (m, 1H), 2.19-2.08 (m, 1H), 1.17 (d, J=6.1 Hz, 6H), 0.90-0.80 (m, 2H).

### Example 211: trans-5-(2-(3-(3-(tert-Butylsulfonyl)azetidin-1-yl)-4,5-difluorophenyl)cyclopropyl)-2,2′-bipyrimidine

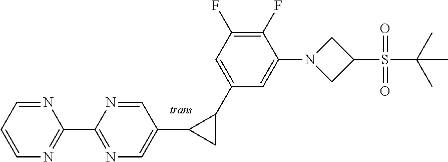 [SMILES: CC(C)(C)S(=O)(=O)C1CN(c2cc(C3CC3c3cnc(-c4ncccn4)nc3)cc(F)c2F)C1]

m/z: 486 [M+H]⁺ observed. ¹H NMR (400 MHz, CDCl₃): δ 9.02 (d, J=4.8 Hz, 2H), 8.76 (s, 2H), 7.42 (t, J=4.9 Hz, 1H), 6.39 (s, 1H), 6.06 (d, J=7.2 Hz, 1H), 4.42-4.23 (m, 5H), 2.30-2.13 (m, 2H), 1.40 (s , 9H), 0.86-0.84 (m, 2H).

### Example 212: trans-5-(2-(3,4-Difluoro-5-(3-(2-methoxyethoxy)azetidin-1-yl)phenyl)cyclopropyl)-2,2′-bipyrimidine

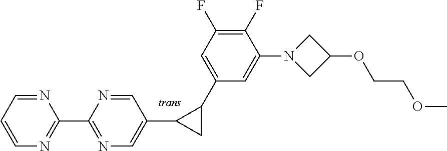 [SMILES: COCCOC1CN(c2cc(C3CC3c3cnc(-c4ncccn4)nc3)cc(F)c2F)C1]

m/z: 440 [M+H]⁺ observed. ¹H NMR (400 MHz, CDCl₃) δ 9.01 (d, J=4.8 Hz, 2H), 8.76 (s, 2H), 7.42 (t, J=4.8 Hz, 1H), 6.30 (ddd, J=10.9, 6.3, 2.1 Hz, 1H), 6.03 (d, J=7.3 Hz, 1H), 4.53-4.40 (m, 1H), 4.23 (d, J=6.3 Hz, 2H), 3.92-3.83 (m, 2H), 3.61-3.50 (m, 4H), 3.39 (s, 3H), 2.28-2.20 (m, 1H), 2.14 (d, J=10.7 Hz, 1H), 0.86-0.85 (m, 2H).

### Example 213: trans-5-(2-(3-(3-(3,4-Difluoro-5-methoxyphenyl)azetidin-1-yl)-4,5-difluorophenyl)cyclopropyl)-2,2′-bipyrimidine

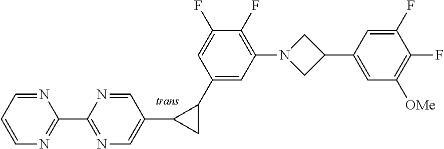 [SMILES: COc1cc(C2CN(c3cc(C4CC4c4cnc(-c5ncccn5)nc4)cc(F)c3F)C2)cc(F)c1F]

m/z: 508 [M+H]⁺ observed.' H NMR (400 MHz, CDCl₃): δ 9.01 (d, J=4.8 Hz, 2H), 8.76 (s, 2H), 7.42 (t, J=4.8 Hz, 1H), 6.81 (ddd, J=10.6, 6.4, 2.1 Hz, 1H), 6.74 (dt, J=6.7, 2.0 Hz, 1H), 6.34 (ddd, J=10.9, 6.4, 2.2 Hz, 1H), 6.08 (dt, J=7.3, 2.0 Hz, 1H), 4.41 (td, J=7.9, 2.3 Hz, 2H), 3.97 (ddt, J=7.7, 6.0, 1.7 Hz, 2H), 3.91 (s, 3H), 3.90-3.78 (m, 1H), 2.31-2.21 (m, 1H), 2.21-2.13 (m, 1H), 1.62-1.58 (m , 2H).

### Example 214: trans-5-(2-(3-(3-(3,4-difluorophenyl)azetidin-1-yl)-4,5-difluorophenyl)cyclopropyl)-2,2′-bipyrimidine

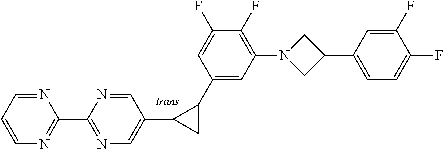 [SMILES: Fc1ccc(C2CN(c3cc(C4CC4c4cnc(-c5ncccn5)nc4)cc(F)c3F)C2)cc1F]

m/z: 478 [M+H]⁺ observed. ¹H NMR (400 MHz, CDCl₃): δ 9.01 (d, J=4.9 Hz, 2H), 8.75 (s, 2H), 7.42 (t, J=4.8 Hz, 1H), 7.22 (ddd, J=11.3, 7.5, 2.1 Hz, 1H), 7.17-6.99 (m, 3H), 6.34 (ddd, J=10.9, 6.4, 2.2 Hz, 1H), 6.07 (dt, J=7.3, 1.9 Hz, 1H), 4.42 (td, J=7.8, 2.2 Hz, 2H), 3.97 (ddt, J=7.6, 5.9, 1.7 Hz, 2H), 3.91-3.80 (m, 1H), 2.32-2.19 (m, 1H), 2.18-2.12 (m, 1H), 1.59-1.61 (m, 2H).

### Example 215: trans-5-(2-(3,4-Difluoro-5-(3-(4-fluoro-3-methoxyphenyl)azetidin-1-yl)phenyl)cyclopropyl)-2,2′-bipyrimidine

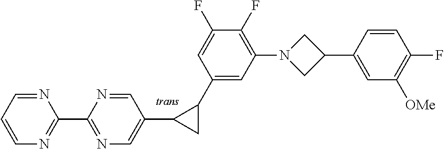 [SMILES: COc1cc(C2CN(c3cc(C4CC4c4cnc(-c5ncccn5)nc4)cc(F)c3F)C2)ccc1F]

m/z: 490 [M+H]⁺ observed. ¹H NMR (400 MHz, CDCl₃): δ 9.01 (d, J=4.8 Hz, 2H), 8.76 (s, 2H), 7.42 (t, J=4.9 Hz, 1H), 7.09-7.00 (m, 1H), 6.97 (dd, J=8.1, 2.2 Hz, 1H), 6.88 (ddd, J=8.4, 4.3, 2.1 Hz, 1H), 6.33 (ddd, J=10.8, 6.4, 2.1 Hz, 1H), 6.09 (d, J=7.4 Hz, 1H), 4.42 (td, J=7.8, 2.3 Hz, 2H), 4.00 (dd, J=7.6, 6.1 Hz, 2H), 3.93-3.83 (m, 4H), 2.29-2.21 (m, 1H), 2.17 (ddd, J=8.8, 6.6, 4.5 Hz, 1H), 1.62-1.59 (m, 2H).

### Example 216: trans-5-(2-(3-(3-(3,4-Dimethoxyphenyl)azetidin-1-yl)-4,5-difluorophenyl)cyclopropyl)-2,2′-bipyrimidine

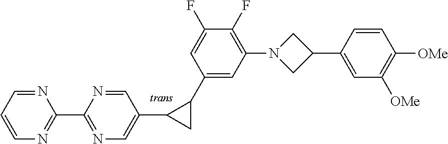 [SMILES: COc1ccc(C2CN(c3cc(C4CC4c4cnc(-c5ncccn5)nc4)cc(F)c3F)C2)cc1OC]

m/z: 502 [M+H]⁺ observed. ¹H NMR (400 MHz, CDCl₃): δ 9.01 (d, J=4.8 Hz, 2H), 8.76 (s, 2H), 7.42 (t, J=4.8 Hz, 1H), 6.90 (d, J=7.4 Hz, 2H), 6.87-6.81 (m, 1H), 6.32 (ddd, J=10.9, 6.3, 2.1 Hz, 1H), 6.09 (dt, J=7.2, 1.9 Hz, 1H), 4.41 (td, J=7.8, 2.3 Hz, 2H), 4.03-3.95 (m, 2H), 3.90-3.80 (m , 7H), 2.25 (td, J=7.4, 4.5 Hz, 1H), 2.21-2.12 (m, 1H), 1.62-1.55 (m, 2H).

### Example 217: trans-5-(2-(3,4-Difluoro-5-((1-methyl-1H-1,2,4-triazol-3-yl)methoxy)phenyl)cyclopropyl)-2,2′-bipyrimidine

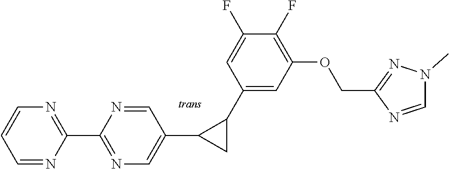 [SMILES: Cn1cnc(COc2cc(C3CC3c3cnc(-c4ncccn4)nc3)cc(F)c2F)n1]

To a microwave vial equipped with a stir bar was added trans-5-(2-(3,4,5-trifluorophenyl)cyclopropyl)-2,2′-bipyrimidine (20.0 mg, 0.06 mmol), (1-methyl-1H-1,2,4-triazol-3-yl)methanol (25.0 mg, 0.22 mmol), KOH (14.0 mg , 0.25 mmol) and DMSO (0.5 mL). The flask was sealed and the mixture was heated thermally at 135° C. overnight. The crude reaction mixture was passed through a syringe filter and purified by reverse phase HPLC to give trans-5-(2-(3,4-difluoro-5#1-methyl-1H-1,2,4-triazol-3-yl)methoxy)phenyl)cyclopropyl)-2,2′-bipyrimidine as the TFA salt (12.9 mg, 39% yield, m/z: 422 [M+H]⁺ observed). ¹H NMR (400 MHz, CDCl3-d) δ 9.09 (d, J=4.9 Hz, 2H), 8.84 (s, 2H), 8.47 (s, 1H), 7.56 (t, J=4.9 Hz, 1H), 6.75 (dt, J=6.6, 1.9 Hz, 1H), 6.63 (ddd, J=10.6, 6.3, 2.1 Hz, 1H), 5.29 (s, 2H), 4.01 (s, 3H), 2.33 (ddd, J=8.8, 6.4, 4.5 Hz, 1H), 2.28-2.15 (m, 1H), 1.66 (ddt, J=11.2, 8.7, 6.1 Hz, 2H).

The following examples were prepared in a similar manner as trans-5-(2-(3,4-difluoro-5-((1-methyl-1H-1,2,4-triazol-3-yl)methoxy)phenyl)cyclopropyl)-2,2′-bipyrimidine from trans-5-(2-(3,4,5-trifluorophenyl)cyclopropyl)-2,2′-bipyrimidine and an appropriate alcohol.

### Example 218: trans-2-((5-(2-([2,2′-Bipyrimidin]-5-yl)cyclopropyl)-2,3-difluorophenoxy)methyl)thiazole

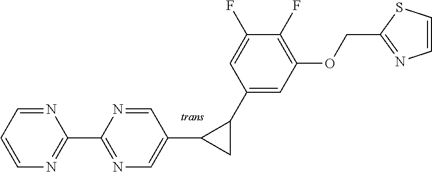 [SMILES: Fc1cc(C2CC2c2cnc(-c3ncccn3)nc2)cc(OCc2nccs2)c1F]

m/z: 424 [M+H]⁺ observed. ¹H NMR (400 MHz, CDCl₃): δ 9.54 (s, 1H), 9.08 (s, 2H), 8.83 (s, 2H), 7.91 (d, J=3.4 Hz, 1H), 7.53 (d, J=3.4 Hz, 1H), 6.74-6.69 (m, 1H), 6.67 (ddd, J=10.5, 6.3, 2.0 Hz, 1H), 5.58 (s, 2H), 2.37-2.26 (m, 1H), 2.22 (s, 1H), 1.64 (ddt, J=12.2, 9.1, 6.0 Hz, 2H).

### Example 219: trans-5-(2-(3,4-Difluoro-5-((1-methyl-1H-pyrazol-3-yl)methoxy)phenyl)cyclopropyl)-2,2′-bipyrimidine

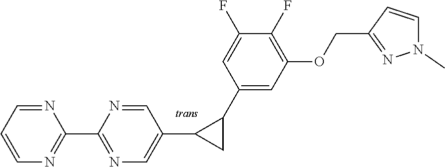 [SMILES: Cn1ccc(COc2cc(C3CC3c3cnc(-c4ncccn4)nc3)cc(F)c2F)n1]

m/z: 421 [M+H]⁺ observed. ¹H NMR (400 MHz, CDCl₃): δ 9.05 (s, 2H), 8.80 (s, 2H), 7.56-7.45 (m, 1H), 7.37 (d, J=2.3 Hz, 1H), 6.72 (dt, J=6.7, 2.0 Hz, 1H), 6.58 (ddd, J=10.7, 6.4, 2.1 Hz, 1H), 6.40 (d, J=2.3 Hz, 1H), 5.19 (s, 2H), 3.93 (s, 3H), 2.30 (ddd, J=8.8, 6.2, 4.5 Hz, 1H), 2.20 (dt, J=9.8, 5.5 Hz, 1H), 1.62 (dp, J=8.8, 6.0 Hz, 2H).

### Example 220: trans-5-(2-(3-(3,3-Dimethylpyrrolidin-1-yl)-4,5-difluorophenyl)cyclopropyl)-2,2′-bipyrimidine (single enantiomer I)

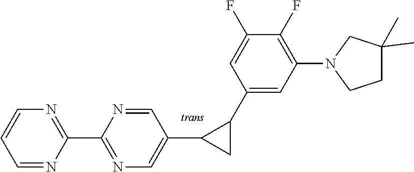 [SMILES: CC1(C)CCN(c2cc(C3CC3c3cnc(-c4ncccn4)nc3)cc(F)c2F)C1]

### Example 221: trans-5-(2-(3-(3,3-Dimethylpyrrolidin-1-yl)-4,5-difluorophenyl)cyclopropyl)-2,2′-bipyrimidine (single enantiomer II)

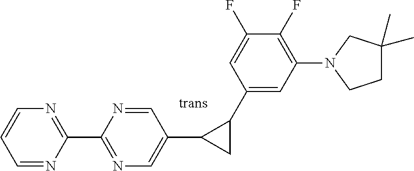 [SMILES: CC1(C)CCN(c2cc(C3CC3c3cnc(-c4ncccn4)nc3)cc(F)c2F)C1]

### 1-(5-Bromo-2,3-fluorophenyl)-3,3-dimethylpyrrolidine

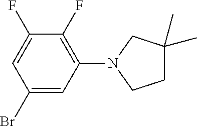 [SMILES: CC1(C)CCN(c2cc(Br)cc(F)c2F)C1]

A mixture of 5-bromo-1,2,3-trifluorobenzene (1 g, 4.74 mmol), 3,3-dimethylpyrrolidine hydrochloride salt (643 mg, 4.74 mmol) and potassium carbonate (1.31 g, 9.48 mmol) in DMSO (10 mL) was heated to 90° C. for 3 hr under N₂. After cooling, to the mixture was added H₂O (40 mL) and extracted with EtOAc (2×40 mL). The combined organic layer was washed with saturated aqueous brine solution (100 mL) then concentrated under vacuum. The reaction was purified by normal phase SiO₂ chromatography (100% petroleum ether) to afford 1-(5-bromo-2,3-difluorophenyl)-3,3-dimethylpyrrolidine as a colorless oil (630 mg, 45% yield). ¹H NMR (400 MHz, CDCl₃): δ 6.59-6.55 (m, 1H), 6.44-6.42 (m, 1H), 3.52-3.48 (m, 2H), 3.15 (d, J=2.8 Hz, 2H), 1.72 (t, J=7.2 Hz, 2H), 1.12 (s, 6H).

### trans-2-Chloro-5-(2-(3-(3,3-dimethylpyrrolidin-1-yl)-4,5-difluorophenyl)cyclopropyl)pyrimidine

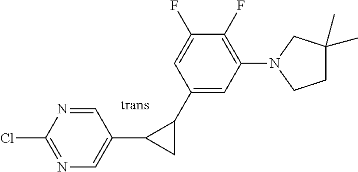 [SMILES: CC1(C)CCN(c2cc(C3CC3c3cnc(Cl)nc3)cc(F)c2F)C1]

To a mixture of 1-(5-bromo-2,3-difluorophenyl)-3,3-dimethylpyrrolidine (890 mg, 3.07 mmol) and trans-2-chloro-5-(2-(4 ,4 ,5,5-tetramethyl-1,3,2-dioxaborolan-2-yl)cyclopropyl)pyrimidine (1.29 g, 4.60 mmol) in THF-H₂O (4:1, 25 mL) was added Cs₂CO₃ (2.0 g, 6.1 mmol), followed by Pd(dppf)Cl₂. CH₂Cl₂ (251 mg, 0.31 mmol) under N₂. The reaction mixture was heated at 80° C. for 12 hr. After cooling, H₂O (50 mL) was added and the mixture was extracted with EtOAc (3×50 mL). The organic layer was concentrated under vacuum. The reaction was purified by normal phase SiO₂ chromatography (0-10% EtOAc/petroleum ether1) to afford trans-2-chloro-5-(2-(3-(3,3-dimethylpyrrolidin-1-yl)-4,5-difluorophenyl)cyclopropyl) pyrimidine as a white solid (0.56 g, 50% yield). ¹H NMR (400 MHz, CDCl₃): δ 8.35-8.27 (m, 2H), 6.11-6.05 (m, 2H), 3.48-3.44 (m, 2H), 3.10 (d, J=2.4 Hz, 2H), 2.05-1.96 (m, 2H), 1.68-1.64 (m, 2H), 1.47-1.39 (m, 2H), 1.10 (s, 6H). 5-(2-(3-(3,3-Dimethylpyrrolidin-1-yl)-4,5-difluorophenyl)cyclopropyl)-2,2′-bipyrimidine:

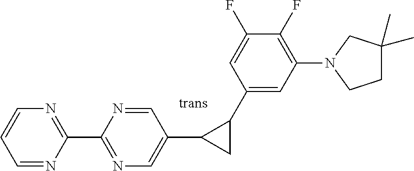 [SMILES: CC1(C)CCN(c2cc(C3CC3c3cnc(-c4ncccn4)nc3)cc(F)c2F)C1]

To a mixture of trans-2-chloro-5-(2-(3-(3,3-dimethylpyrrolidin-1-yl)-4,5-difluorophenyl) cyclopropyl)pyrimidine (610 mg, 1.68 mmol) in 1,4-dioxane (10 mL) was added 2-(tributylstannyl)pyrimidine (0.75 mL, 2.35 mmol), copper(I) iodide (31.9 mg, 0.17 mmol) and Pd(dppf)Cl₂ (123 mg, 0.17 mmol) under N₂. Then the mixture was heated to 110° C. for 12 hr. The reaction mixture was purified directly by normal phase SiO₂ chromatography (0-40% MeOH/EtOAc) to give a semi-purified product which was further purified by reverse phase HPLC to give trans-5-(2-(3-(3,3-dimethylpyrrolidin-1-yl)-4,5-difluorophenyl)cyclopropyl)-2,2′-bipyrimidine as a light red solid (400 mg, 58% yield). ¹H NMR (400 MHz, DMSO): 6 8.99 (d, J=4.8 Hz, 2H), 8.83 (s, 2H), 7.63 (t, J=4.8 Hz, 1H), 6.49-6.45 (m, 1H), 6.37 (d, J=7.6 Hz, 1H), 3.51-3.48 (m, 2H), 3.15 (d, J=1.6 Hz, 2H), 2.48-2.45 (m, 1H), 2.44-2.33 (m, 1H), 1.73-1.61 (m, 4H), 1.10 (s, 6H).

A mixture of enantiomers of trans-5-(2-(3-(3,3-dimethylpyrrolidin-1-yl)-4,5-difluorophenyl)cyclopropyl)-2,2′-bipyrimidine (400 mg) was separated by SFC (supercritical fluid chromatography) on a CHIRALCEL° OJ column using liquid CO₂ and MeOH [0.1% aqueous NH₃ as modifier] (69:31) to give trans-5-(2-(3-(3,3-dimethylpyrrolidin-1-yl)-4,5-difluorophenyl)cyclopropyl)-2,2′-bipyrimidine (single enantiomer I) as a white solid (faster eluting enantiomer, 98 mg, 24% yield, m/z: 408 [M+H]⁺ observed), and trans-5-(2-(3-(3,3-dimethylpyrrolidin-1-yl)-4,5-difluorophenyl)cyclopropyl)-2,2′-bipyrimidine (single enantiomer II) as a white solid (slower eluting enantiomer, 93 mg, 23% yield, m/z: 408 [M+H]⁺ observed).

### Example 220: trans-5-(2-(3-(3,3-Dimethylpyrrolidin-1-yl)-4,5-difluorophenyl)cyclopropyl)-2,2′-bipyrimidine (single enantiomer I)

m/z: 408 [M+H]⁺ observed. ¹H NMR (400 MHz, DMSO-d₆): δ 9.01 (d, J=4.8 Hz, 2H), 8.84 (s, 2H), 7.64 (t, J=4.8 Hz, 1H), 6.50-6.46 (m, 1H), 6.38 (d, J=8.0 Hz, 1H), 3.52-3.48 (m, 2H), 3.16 (d, J=2.4 Hz, 2H), 2.47-2.44 (m, 1H), 2.38-2.34 (m, 1H), 1.74-1.62 (m, 4H), 1.11 (s, 6H).

### Example 221: trans-5-(2-(3-(3,3-Dimethylpyrrolidin-1-yl)-4,5-difluorophenyl)cyclopropyl)-2,2′-bipyrimidine (single enantiomer II)

m/z: 408 [M+H]⁺ observed. ¹H NMR (400 MHz, DMSO-d₆): δ 9.01 (d, J=4.8 Hz, 2H), 8.84 (s, 2H), 7.64 (t, J=4.8 Hz, 1H), 6.50-6.46 (m, 1H), 6.38 (d, J=8.0 Hz, 1H), 3.52-3.48 (m, 2H), 3.16 (d, J=2.4 Hz, 2H), 2.47-2.44 (m, 1H), 2.38-2.34 (m, 1H), 1.74-1.62 (m, 4H), 1.11 (s, 6H).

The following examples were prepared in a similar manner as trans-5-(2-(3-(3,3-dimethylpyrrolidin-1-yl)-4,5-difluorophenyl)cyclopropyl)-2,2′-bipyrimidine from 5-bromo-1,2,3-trifluorobenzene and an appropriate amine or an appropriate alcohol.

### Example 222: trans-6-(5-(2-([2,2′-Bipyrimidin]-5-yl)cyclopropyl)-2,3-difluorophenyl)-2-oxa-6-azaspiro[3.4]octane (single enantiomer I)

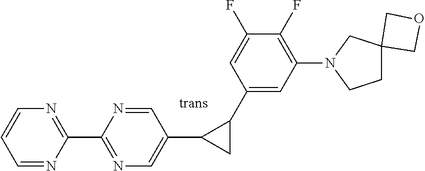 [SMILES: Fc1cc(C2CC2c2cnc(-c3ncccn3)nc2)cc(N2CCC3(COC3)C2)c1F]

### Example 223: trans-6-(5-(2-([2,2′-Bipyrimidin]-5-yl)cyclopropyl)-2,3-difluorophenyl)-2-oxa-6-azaspiro[3.4]octane (single enantiomer II)

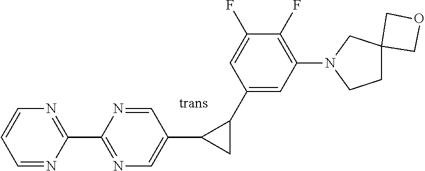 [SMILES: Fc1cc(C2CC2c2cnc(-c3ncccn3)nc2)cc(N2CCC3(COC3)C2)c1F]

A mixture of enantiomers of trans-6-(5-(2-([2,2′-bipyrimidin]-5-yl)cyclopropyl)-2,3-difluorophenyl)-2-oxa-6-azaspiro[3.4]octane (150 mg) was separated by SFC (supercritical fluid chromatography) on a Phenomenex Cellulose-2® column using liquid CO₂ and MeOH [0.1% aqueous NH₃ as modifier] (40:60) to give trans-6-(5-(2-([2,2′-bipyrimidin]-5-yl)cyclopropyl)-2,3-difluorophenyl)-2-oxa-6-azaspiro[3.4]octane (single enantiomer I) as a white solid (faster eluting enantiomer, 39 mg, 26% yield, m/z: 422 [M+H]⁺), and trans-6-(5-(2-([2,2′-bipyrimidin]-5-yl)cyclopropyl)-2,3-difluorophenyl)-2-oxa-6-azaspiro[3.4]octane (single enantiomer II) as a white solid (slower eluting enantiomer, 43 mg, 29% yield, m/z: 422 [M+H]⁺).

### Example 222: trans-6-(5-(2-([2,2′-Bipyrimidin]-5-yl)cyclopropyl)-2,3-difluorophenyl)-2-oxa-6-azaspiro[3.4]octane (single enantiomer I)

m/z: 422 [M+H]⁺. ¹H NMR (400 MHz, DMSO-d₆): δ 8.99 (d, J=4.8 Hz, 2H), 8.83 (s, 2H), 7.63 (t, J=4.8 Hz, 1H), 6.55-6.51 (m, 1H), 6.41 (d, J=7.2 Hz, 1H), 4.57 (d, J=6 Hz, 2H), 4.51 (d, J=6 Hz, 2H), 3.62 (d, J=2.4 Hz, 2H), 3.39-3.38 (m, 2H), 2.46-2.43 (m, 1H), 2.36-2.33 (m, 1H), 2.20 (t, J=6.8 Hz, 2H), 1.72-1.69 (m, 1H), 1.65-1.62 (m, 1H).

### Example 223: trans-6-(5-(2-([2,2′-Bipyrimidin]-5-yl)cyclopropyl)-2,3-difluorophenyl)-2-oxa-6-azaspiro[3.4]octane (single enantiomer II)

m/z: 422 [M+H]t ¹H NMR (400 MHz, DMSO-d₆): δ 8.99 (d, J=4.8 Hz, 2H), 8.83 (s, 2H), 7.63 (t, J=4.8 Hz, 1H), 6.55-6.51 (m, 1H), 6.41 (d, J=7.2 Hz, 1H), 4.57 (d, J=6 Hz, 2H), 4.51 (d, J=6 Hz, 2H), 3.62 (d, J=2.4 Hz, 2H), 3.39-3.38 (m, 2H), 2.46-2.43 (m, 1H), 2.36-2.33 (m, 1H), 2.20 (t, J=6.8 Hz, 2H), 1.72-1.69 (m, 1H), 1.65-1.62 (m, 1H).

### Example 224: trans-1-((3S)-3-((5-(2-([2,2′-Bipyrimidin]-5-yl)cyclopropyl)-2,3-difluorophenyl)amino)pyrrolidin-1-yl)ethanone(single diastereomer I)

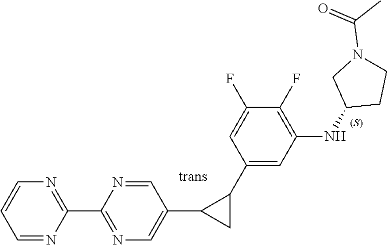

### Example 225: trans-1-((3S)-3-((5-(2-([2,2′-Bipyrimidin]-5-yl)cyclopropyl)-2,3-difluorophenyl)amino)pyrrolidin-1-yl)ethanone (single diastereomer II)

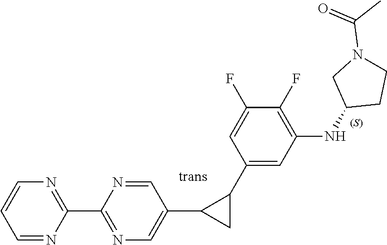

A mixture of diastereomers of trans-1-((3S)-3-((5-(2-([2,2′-bipyrimidin]-5-yl)cyclopropyl)-2,3-difluorophenyl)amino)pyrrolidin-1-yl)ethanone (200 mg) was separated by SFC on a CHIRALCEL® OD column using liquid CO₂ and MeOH [0.1% aqueous NH₃ modifier] (50:50) to give trans-1-((3S)-3-((5-(2-([2,2′-bipyrimidin]-5-yl)cyclopropyl)-2,3-difluorophenyl)amino)pyrrolidin-1-yl)ethanone (single diastereomer I) as a white solid (faster eluting diastereomer, 80 mg, 40% yield, m/z: 437 [M+H]⁺ observed) and trans-1-((3S)-3-((5-(2-([2,2′-bipyrimidin]-5-yl)cyclopropyl)-2,3-difluorophenyl)amino)pyrrolidin-1-yl)ethanone (single diastereomer II) (slower eluting diastereomer, 90 mg, 45% yield, m/z: 437 [M+H]⁺ observed).

### Example 224: trans-1-((3S)-3-((5-(2-([2,2′-Bipyrimidin]-5-yl)cyclopropyl)-2,3-difluorophenyl)amino)pyrrolidin-1-yl)ethenone (single diastereomer I)

m/z: 437 [M+H]⁺ observed. ¹H NMR (400 MHz, DMSO-d₆): δ 8.99 (d, J=4.8 Hz, 2H), 8.83 (s, 2H), 7.62 (t, J=4.8 Hz, 1H), 6.52 (t, J=8 Hz, 1H), 6.45 (m, 1H), 5.92 (dd, J₁=6.4 Hz, J₂=23.2 Hz, 1H), 4.19-4.14 (m, 1H), 3.80-3.77 (m, 0.5H), 3.63-3.58 (m, 1H), 3.49-3.47 (m, 1H), 3.25-3.24 (m, 0.5H), 2.45-2.44 (m, 1H), 2.35-2.32 (m, 1H), 2.30-2.22 (m, 1H), 1.93-1.92 (m, 4H), 1.72-1.62 (m, 2H).

### Example 225: trans-1-((3S)-3-((5-(2-([2,2′-Bipyrimidin]-5-yl)cyclopropyl)-2,3-difluorophenyl)amino)pyrrolidin-1-yl)ethanone (single diastereomer II)

m/z: 437 [M+H]⁺ observed. ¹H NMR (400 MHz, DMSO-d₆): δ 8.99 (d, J=4.8 Hz, 2H), 8.83 (s, 2H), 7.62 (t, J=4.8 Hz, 1H), 6.52 (t, J=8 Hz, 1H), 6.45 (m, 1H), 5.92 (dd, J₁=6.4 Hz, J₂=23.2 Hz, 1H), 4.19-4.14 (m, 1H), 3.80-3.77 (m, 0.5H), 3.63-3.58 (m, 1H), 3.49-3.47 (m, 1H), 3.25-3.24 (m, 0.5H), 2.45-2.44 (m, 1H), 2.35-2.32 (m, 1H), 2.30-2.22 (m, 1H), 1.93-1.92 (m, 4H), 1.72-1.62 (m, 2H).

### Example 226: trans-1-(3R)-3-((5-(2-([2,2′-Bipyrimidin]-5-yl)cyclopropyl)-2,3-difluorophenyl) amino) pyrrolidin-1-yl)ethenone (single diastereomer I)

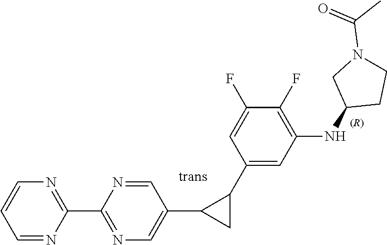

### Example 227: trans-1-(3R)-3-((5-(2-([2,2′-Bipyrimidin]-5-yl)cyclopropyl)-2,3-difluorophenyl) amino) pyrrolidin-1-yl)ethanone (single diastereomer II)

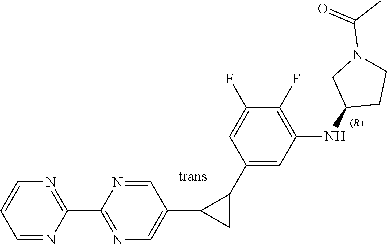

A mixture of diastereomers of trans-1-[(3R)-3-[2,3-difluoro-5-[2-(2-pyrimidin-2-ylpyrimidin-5-yl)cyclopropyl]anilino]pyrrolidin-1-yl]ethanone (210 mg) was separated by SFC on a Phenomenex Cellulose-2® column using liquid CO₂ and MeOH [0.1% aqueous NH₃ modifier] (40:60) to give trans-1-[(3R)-3-[2,3-difluoro-5-[2-(2-pyrimidin-2-ylpyrimidin-5-yl)cyclopropyl]anilino]pyrrolidin-1-yl]ethenone (single diastereomer I) as a white solid (faster eluting diastereomer, 44 mg, 21% yield, m/z: 437[M+H]⁺ observed), and trans-1-[(3R)-3-[2,3-difluoro-5-[2-(2-pyrimidin-2-ylpyrimidin-5 yl)cyclopropyl]anilino]pyrrolidin-1-yl]ethanone (single diastereomer II) as a white solid (slower eluting diastereomer, 55 mg, 26% yield, m/z: 437[M+H]⁺ observed).

### Example 226: trans-1-((3R)-3-((5-(2-([2,2′-Bipyrimidin]-5-yl)cyclopropyl)-2,3-difluorophenyl) amino) pyrrolidin-1-yl)ethenone (single diastereomer I)

m/z: 437[M+H]⁺ observed. ¹H NMR (400 MHz, DMSO-d₆): δ 8.99 (d, J=4.8 Hz, 2H), 8.83 (s, 2H), 7.62 (t, J=4.8 Hz, 1H), 6.54 (t, J=8.4 Hz, 1H), 6.45-6.41 (m, 1H), 5.92 (dd, J=23.2 Hz, J=6.4 Hz, 1H), 4.17-4.04 (m, 1H), 3.83-3.80 (m, 1.5H), 3.62-3.60 (m, 1H), 3.48-3.40 (m, 0.5H), 3.33-3.24 (m, 1H), 2.50-2.44 (m, 1H), 2.37-2.33 (m, 1H), 2.24-2.06 (m, 1H), 2.00-1.86 (m, 4H), 1.73-1.61 (m, 2H).

### Example 227: trans-1-((3R)-3-((5-(2-([2,2′-Bipyrimidin]-5-yl)cyclopropyl)-2,3-difluorophenyl) amino) pyrrolidin-1-yl)ethanone (single diastereomer II)

m/z: 437[M+H]⁺ observed. ¹H NMR (400 MHz, DMSO-d₆): δ 8.99 (d, J=4.8 Hz, 2H), 8.83 (s, 2H), 7.62 (t, J=4.8 Hz, 1H), 6.54 (t, J=8.4 Hz, 1H), 6.45-6.41 (m, 1H), 5.92 (dd, J=23.2 Hz, J=6.4 Hz, 1H), 4.17-4.04 (m, 1H), 3.83-3.80 (m, 1.5H), 3.62-3.60 (m, 1H), 3.48-3.40 (m, 0.5H), 3.33-3.24 (m, 1H), 2.50-2.44 (m, 1H), 2.37-2.33 (m, 1H), 2.24-2.06 (m, 1H), 2.00-1.86 (m, 4H), 1.73-1.61 (m, 2H).

### Example 228: trans-(3S)-N-(5-(2-([2,2′-Bipyrimidin]-5-yl)cyclopropyl)-2,3-difluorophenyl)-1-methylpyrrolidin-3-amine (single diastereomer I)

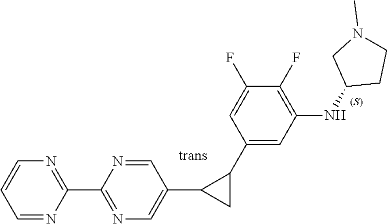

### Example 229: trans-(3S)-N-(5-(2-([2,2′-Bipyrimidin]-5-yl)cyclopropyl)-2,3-difluorophenyl)-1-methylpyrrolidin-3-amine (single diastereomer II)

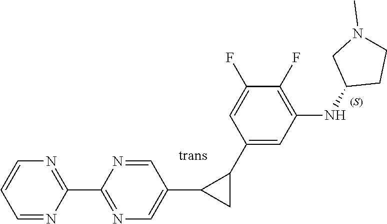

A mixture of diastereomers of trans-(3S)-N-(5-(2-([2,2′-bipyrimidin]-5-yl)cyclopropyl)-2,3-difluorophenyl)-1-methylpyrrolidin-3-amine (50 mg) was separated by SFC (supercritical fluid chromatography) on a CHIRALCEL ®OD column using liquid CO₂ and MeOH [0.1% aqueous NH₃ modifier] (50:50) to give trans-(3S)-N-(5-(2-([2,2′-bipyrimidin]-5-yl)cyclopropyl)-2,3-difluorophenyl)-1-methylpyrrolidin-3-amine (single diastereomer I) as a white solid (faster eluting diastereomer, 9 mg, 17% yield, m/z: 409 [M+H]⁺ observed) and trans-(3S)-N-(5-(2-([2,2′-bipyrimidin]-5-yl)cyclopropyl)-2,3-difluorophenyl)-1-methylpyrrolidin-3-amine (single diastereomer II) as a white solid (slower eluting diastereomer, 12 mg, 23% yield, m/z: 409 [M+H]⁺ observed).

### Example 228: trans-(3S)-N-(5-(2-([2,2′-Bipyrimidin]-5-yl)cyclopropyl)-2,3-difluorophenyl)-1-methylpyrrolidin-3-amine (single diastereomer I)

m/z: 409 [M+H]⁺ observed. ¹H NMR (400 MHz, DMSO-d₆): δ 8.99 (d, J=4.8 Hz, 2H), 8.83 (s, 2H), 7.62 (t, J=4.8 Hz, 1H), 6.43-6.38 (m, 2H), 5.64 (d, J=7.2 Hz, 1H), 4.01-3.96 (m, 1H), 2.77-2.76 (m, 1H), 2.74-2.55 (m, 1H), 2.44-2.36 (m, 4H), 2.33-2.24 (m, 4H), 1.71-1.67 (m, 2H), 1.60-1.58 (m, 1H).

### Example 229: trans-(3S)-N-(5-(2-([2,2′-Bipyrimidin]-5-yl)cyclopropyl)-2,3-difluorophenyl)-1-methylpyrrolidin-3-amine (single diastereomer II)

m/z: 409 [M+H]⁺ observed. ¹H NMR (400 MHz, DMSO-d₆): δ 8.99 (d, J=4.8 Hz, 2H), 8.83 (s, 2H), 7.62 (t, J=4.8 Hz, 1H), 6.43-6.38 (m, 2H), 5.64 (d, J=7.2 Hz, 1H), 4.01-3.96 (m, 1H), 2.77-2.76 (m, 1H), 2.74-2.55 (m, 1H), 2.44-2.36 (m, 4H), 2.33-2.24 (m, 4H), 1.71-1.67 (m, 2H), 1.60-1.58 (m, 1H).

### Example 230: trans-(3R)-N-(5-(2-([2,2′-Bipyrimidin]-5-yl)cyclopropyl)-2,3-difluorophenyl)-1-methylpyrrolidin-3-amine(single diastereomer I)

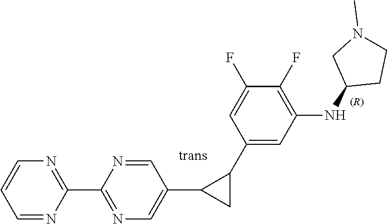

### Example 231: trans-(3R)-N-(5-(2-([2,2′-Bipyrimidin]-5-yl)cyclopropyl)-2,3-difluorophenyl)-1-methylpyrrolidin-3-amine(single diastereomer II)

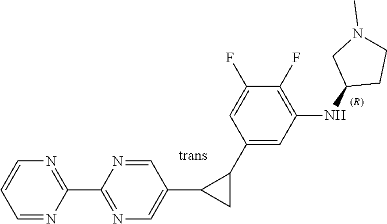

A mixture of diastereomers of trans-(3R)-N-(5-(2-([2,2′-bipyrimidin]-5-yl)cyclopropyl)-2,3-difluorophenyl)-1-methylpyrrolidin-3-amine (70 mg) was separated by SFC (supercritical fluid chromatography) on a CHIRALCEL® OD column using liquid CO₂ and MeOH [0.1% aqueous NH₃ modifier] (50:50) to give trans-(3R)-N-(5-(2-([2,2′-bipyrimidin]-5-yl)cyclopropyl)-2,3-difluorophenyl)-1-methylpyrrolidin-3-amine (single diastereomer I) as a white solid (faster eluting diastereomer, 15 mg, 21% yield, m/z: 409[M+H]⁺ observed), and trans-(3R)-N-(5-(2-([2,2′-bipyrimidin]-5-yl) cyclopropyl)-2,3-difluorophenyl)-1-methylpyrrolidin-3-amine (single diastereomer II) as white solid (slower eluting diastereomer, 25 mg, 35% yield, m/z: 409[M+H]⁺ observed).

### Example 230: trans-(3R)-N-(5-(2-([2,2′-Bipyrimidin]-5-yl)cyclopropyl)-2,3-difluorophenyl)-1-methylpyrrolidin-3-amine(single diastereomer I)

m/z: 409[M+H]⁺ observed. ¹H NMR (400 MHz, DMSO-d₆): δ 8.99 (d, J=4.4 Hz, 2H), 8.83 (s, 2H), 7.62 (t, J=5.6 Hz, 1H), 6.44-6.38 (m, 2H), 5.68 (d, J=6.8 Hz, 1H), 4.07 (s, 1H), 2.82 (t, J=8.4 Hz, 1H), 2.62-2.44 (m, 5H), 2.35-2.20 (m, 4H), 1.70-1.67 (m, 2H), 1.62-1.59 (m, 1H).

### Example 231: trans-(3R)-N-(5-(2-([2,2′-bipyrimidin]-5-yl)cyclopropyl)-2,3-difluorophenyl)-1-methylpyrrolidin-3-amine(single diastereomer II)

m/z: 409[M+H]⁺ observed. ¹H NMR (400 MHz, DMSO-d₆): δ 8.99 (d, J=4.4 Hz, 2H), 8.83 (s, 2H), 7.62 (t, J=5.6 Hz, 1H), 6.44-6.38 (m, 2H), 5.68 (d, J=6.8 Hz, 1H), 4.07 (s, 1H), 2.82 (t, J=8.4 Hz, 1H), 2.62-2.44 (m, 5H), 2.35-2.20 (m, 4H), 1.70-1.67 (m, 2H), 1.62-1.59 (m, 1H).

### Example 232: trans-5-(2-(3,4-Difluoro-5-(4-methyl-1H-pyrazol-1-yl)phenyl)cyclopropyl)-2,2′-bipyrimidine

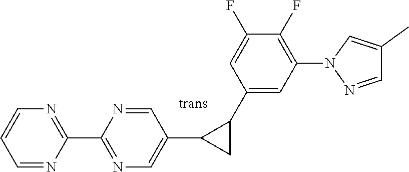 [SMILES: Cc1cnn(-c2cc(C3CC3c3cnc(-c4ncccn4)nc3)cc(F)c2F)c1]

m/z: 391[M+H]⁺ observed. ¹H NMR (400 MHz, CDCl₃): δ 9.00 (d, J=4.8 Hz, 2H), 8.76 (d, J=2.2 Hz, 2H), 7.80 (d, J=3.1 Hz, 1H), 7.55 (s, 1H), 7.50 (dt, J=6.4, 2.1 Hz, 1H), 7.41 (t, J=4.8 Hz, 1H), 6.89 (ddd, J=10.6, 6.8, 2.3 Hz, 1H), 2.36 (ddd, J=8.7, 6.3, 4.5 Hz, 1H), 2.27 (ddd, J=8.7, 6.3, 4.4 Hz, 1H), 2.16 (s, 3H), 1.64 (d, J=3.5 Hz, 1H), 1.41-1.20 (m, 1H).

### Example 233: trans-N-(5-(2-([2,2′-Bipyrimidin]-5-yl)cyclopropyl)-2,3-difluorophenyl)-N-methyloxetan-3-amine

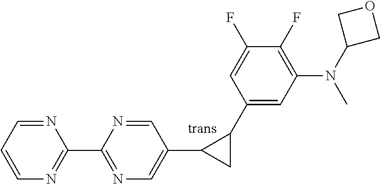 [SMILES: CN(c1cc(C2CC2c2cnc(-c3ncccn3)nc2)cc(F)c1F)C1COC1]

m/z: 396 [M+H]⁺ observed. ¹H NMR (400 MHz, CDCl₃): δ 9.02 (d, J=4.8 Hz, 2H), 8.76 (s, 2H), 7.43 (t, J=4.8 Hz, 1H), 6.58-6.48 (m, 1H), 6.20 (d, J=6.9, 2.0 Hz, 1H), 4.86-4.76 (m, 2H), 4.73-4.64 (m, 2H), 4.59-4.48 (m, 1H), 2.87 (s, 3H), 2.29-2.19 (m, 1H), 2.20-2.10 (m, 1H), 1.72-1.46 (m, 2H).

### Example 234: trans-(1-(5-(2-([2,2′-Bipyrimidin]-5-yl)cyclopropyl)-2,3-difluorophenyl)-1H-pyrazol-4-yl)methanol

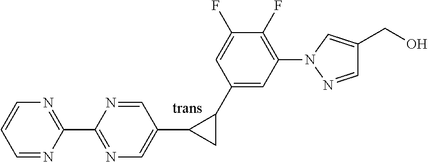 [SMILES: OCc1cnn(-c2cc(C3CC3c3cnc(-c4ncccn4)nc3)cc(F)c2F)c1]

m/z: 407[M+H]⁺ observed. ¹H NMR (400 MHz, CDCl₃): δ 9.01 (d, J=4.8 Hz, 2H), 8.77 (s, 2H), 8.03 (d, J=2.9 Hz, 1H), 7.76 (s, 1H), 7.52 (dt, J=6.2, 2.1 Hz, 1H), 7.42 (t, J=4.8 Hz, 1H), 6.94 (ddd, J=10.5, 6.7, 2.3 Hz, 1H), 4.69 (d, J=5.5 Hz, 2H), 2.37 (td, J=7.9, 7.3, 4.5 Hz, 1H), 2.28 (td, J=8.3, 7.8, 4.5 Hz, 1H), 1.79 (t, J=5.6 Hz, 1H), 1.70-1.67 (m, 1H).

### Example 235: trans-5-(2-(3-((3R,4S)-3,4-Dimethoxypyrrolidin-1-yl)-4,5-difluorophenyl)cyclopropyl)-2,2′-bipyrimidine

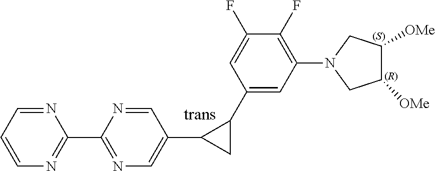

m/z: 440 [M+H]⁺ observed. ¹H NMR (400 MHz, CDCl₃): δ 9.01 (d, J=4.9 Hz, 2H), 8.75 (s, 2H), 7.42 (t, J=4.9 Hz, 1H), 6.32-6.22 (m, 1H), 6.18 (d, J=7.4 Hz, 1H), 3.97 (q, J=4.0 Hz, 2H), 3.67-3.62 (m, 2H), 3.59-3.50 (m, 2H), 3.47 (s, 6H), 2.29-2.19 (m, 1H), 2.21-2.11 (m, 1H), 1.62-1.54 (m, 2H).

### Example 236: trans-5-(2-(3,4-Difluoro-5-(4-(methoxymethyl)-1H-pyrazol-1-yl)phenyl)cyclopropyl)-2,2′-bipyrimidine

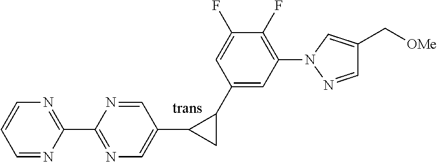 [SMILES: COCc1cnn(-c2cc(C3CC3c3cnc(-c4ncccn4)nc3)cc(F)c2F)c1]

m/z: 421 [M+H]⁺ observed. ¹H NMR (400 MHz, CDCl₃): δ 9.01 (d, J=4.8 Hz, 2H), 8.77 (s, 2H), 8.02 (d, J=2.9 Hz, 1H), 7.74 (s, 1H), 7.53 (dt, J=6.4, 2.1 Hz, 1H), 7.42 (t, J=4.8 Hz, 1H), 6.94 (ddd, J=10.6, 6.7, 2.3 Hz, 1H), 4.44 (s, 2H), 3.40 (s, 3H), 2.43-2.20 (m, 2H), 1.70-1.67 (m, 2H).

### Example 237: trans-1-(5-(2-([2,2′-Bipyrimidin]-5-yl)cyclopropyl)-2,3-difluorophenyl)-5,6-dimethoxy-1H-benzo [d]imidazole

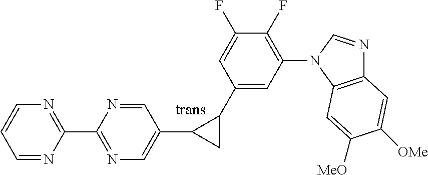 [SMILES: COc1cc2ncn(-c3cc(C4CC4c4cnc(-c5ncccn5)nc4)cc(F)c3F)c2cc1OC]

m/z: 487 [M+H]⁺ observed. ¹H NMR (400 MHz, CDCl₃): δ 9.01 (d, J=1.5 Hz, 2H), 8.78 (s, 2H), 7.91 (d, J=1.4 Hz, 1H), 7.43 (dt, J=5.7, 4.8 Hz, 2H), 7.33 (s, 1H), 7.10 (dd, J=6.0, 2.6 Hz, 1H), 6.78 (d, J=2.0 Hz, 1H), 3.96 (s, 3H), 3.89 (s, 3H), 2.41 (ddd, J=8.9, 6.0, 4.5 Hz, 1H), 2.29 (ddd, J=9.0, 6.0, 4.5 Hz, 1H), 1.75-1.71 (m, 2H).

### Example 238: trans-2-(5-(2-([2,2′-Bipyrimidin]-5-yl)cyclopropyl)-2,3-difluorophenyl)-2-azaspiro[3.3]heptan-6-ol

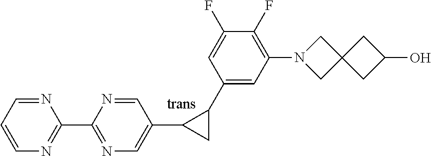 [SMILES: OC1CC2(C1)CN(c1cc(C3CC3c3cnc(-c4ncccn4)nc3)cc(F)c1F)C2]

m/z: 422 [M+H]⁺ observed. ¹H NMR (400 MHz, CDCl₃): δ 9.01 (d, J=4.9 Hz, 2H), 8.75 (s, 2H), 7.42 (t, J=4.8 Hz, 1H), 6.33-6.23 (m, 1H), 5.99 (d, 1H), 4.26 (s, 1H), 3.97 (dd, J=9.1, 2.1 Hz, 4H), 2.66-2.56 (m, 2H), 2.27-2.17 (m, 1H), 2.19-2.09 (m, 3H), 1.75 (s, 1H), 1.57 (t, J=2.3 Hz, 2H).

### Example 239: trans-(3R,4R)-1-(5-(2-([2,2′-Bipyrimidin]-5-yl)cyclopropyl)-2,3-difluorophenyl)pyrrolidine-3,4-diol

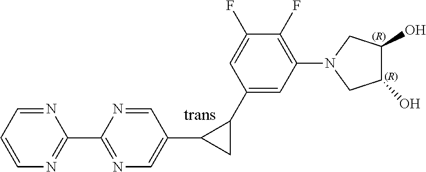

m/z: 412 [M+H]⁺ observed. ¹H NMR (400 MHz, CDCl₃): δ 9.01 (d, J=4.8 Hz, 2H), 8.76 (s, 2H), 7.43 (t, J=4.8 Hz, 1H), 6.35-6.17 (m, 2H), 4.31 (s, 2H), 3.90-3.86 (m, 2H), 3.40 (d, J=11.0 Hz, 2H), 2.31-2.22 (m, 1H), 2.21-2.11 (m, 1H), 1.91 (s, 2H), 1.59 (t, J=7.4 Hz, 2H).

### Example 240: trans-4-(3-(5-(2-([2,2′-Bipyrimidin]-5-yl)cyclopropyl)-2,3-difluorophenoxy)propyl)morpholine

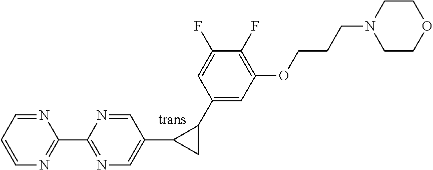 [SMILES: Fc1cc(C2CC2c2cnc(-c3ncccn3)nc2)cc(OCCCN2CCOCC2)c1F]

m/z: 454 [M+H]⁺ observed. ¹H NMR (400 MHz, CDCl₃): δ 9.01 (d, J=4.8 Hz, 2H), 8.76 (s, 2H), 7.47-7.38 (m, 1H), 6.59 (dt, J=6.7, 2.0 Hz, 1H), 6.55-6.48 (m, 1H), 4.11 (t, J=6.3 Hz, 2H), 3.73-3.65 (m, 4H), 2.53 (dd, J=7.6, 6.7 Hz, 2H), 2.46 (t, J=4.5 Hz, 4H), 2.29 (ddd, J=8.7, 6.3, 4.6 Hz, 1H), 2.18 (ddd, J=8.7, 6.1, 4.5 Hz, 1H), 2.05-1.95 (m, 2H), 1.62 (dq, J=8.8, 6.0 Hz, 2H).

### Example 241: trans-4-(3-(4-(2-([2,2′-Bipyrimidin]-5-yl)cyclopropyl)-2,6-difluorophenoxy)propyl)morpholine

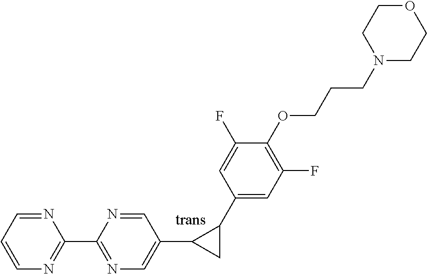 [SMILES: Fc1cc(C2CC2c2cnc(-c3ncccn3)nc2)cc(F)c1OCCCN1CCOCC1]

m/z: 454 [M+H]⁺ observed. ¹H NMR (400 MHz, CDCl₃): δ 9.02 (dd, J=4.8, 0.6 Hz, 2H), 8.76 (d, J=0.6 Hz, 2H), 7.43 (td, J=4.8, 0.7 Hz, 1H), 6.80-6.62 (m, 2H), 4.17 (t, J=6.3 Hz, 2H), 3.71 (t, J=4.7 Hz, 4H), 2.55 (t, J=7.3 Hz, 2H), 2.47 (b.s., 4H), 2.32-2.23 (m, 1H), 2.19 (dt, J=9.0, 5.5 Hz, 1H), 1.93 (p, J=6.6 Hz, 2H), 1.72-1.59 (m, 2H).

### Example 242: trans-5-(2-(4-((2-Oxaspiro[3.3]heptan-6-yl)oxy)-3,5-difluorophenyl)cyclopropyl)-2,2′-bipyrimidine

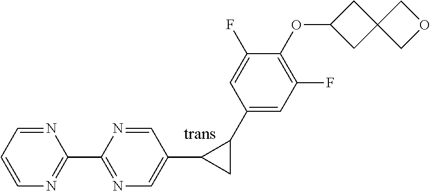 [SMILES: Fc1cc(C2CC2c2cnc(-c3ncccn3)nc2)cc(F)c1OC1CC2(COC2)C1]

m/z: 423 [M+H]⁺ observed. ¹H NMR (400 MHz, CDCl₃): δ 9.02 (d, J=4.9 Hz, 2H), 8.76 (s, 2H), 7.43 (t, J=4.8 Hz, 1H), 6.71 (d, J=8.9 Hz, 2H), 4.69 (d, J=11.9 Hz, 4H), 4.53 (q, J=6.7 Hz, 1H), 2.70-2.60 (m, 2H), 2.45-2.35 (m, 2H), 2.32-2.22 (m, 1H), 2.24-2.14 (m, 1H), 1.70-1.55 (m, 2H).

### Example 243: trans-5-(2-(3-((2-Oxaspiro[3.3]heptan-6-yl)oxy)-4,5-difluorophenyl)cyclopropyl)-2,2′-bipyrimidine

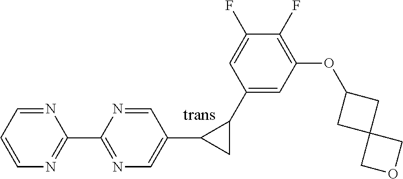 [SMILES: Fc1cc(C2CC2c2cnc(-c3ncccn3)nc2)cc(OC2CC3(COC3)C2)c1F]

m/z: 423 [M+H]⁺ observed. ¹H NMR (400 MHz, CDCl₃): δ 9.02 (d, J=4.9 Hz, 2H), 8.77 (s, 2H), 7.44 (t, J=4.8 Hz, 1H), 6.56-6.51 (m, 1H), 6.40 (d, J=6.6 Hz, 1H), 4.72 (d, J=17.1 Hz, 4H), 4.63-4.52 (m, 1H), 2.85-2.76 (m, 2H), 2.49-2.39 (m, 2H), 2.32-2.22 (m, 1H), 2.21-2.11 (m, 1H), 1.69-1.55 (m, 2H).

### Example 244: trans-2-((5-(2-([2,2′-Bipyrimidin]-5-yl)cyclopropyl)-2,3-difluorophenyl)(methyl)amino)ethan-1-ol

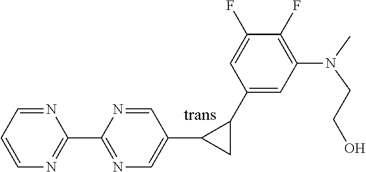 [SMILES: CN(CCO)c1cc(C2CC2c2cnc(-c3ncccn3)nc2)cc(F)c1F]

m/z: 384 [M+H]⁺ observed. ¹H NMR (400 MHz, CDCl₃): δ 9.02 (d, J=4.5 Hz, 2H), 8.77 (s, 2H), 7.43 (t, J=4.8 Hz, 1H), 6.56 (d, J=7.1 Hz, 1H), 6.53-6.43 (m, 1H), 3.82 (t, J=5.5 Hz, 2H), 3.33 (t, J=5.5 Hz, 2H), 2.91 (s, 3H), 2.33-2.23 (m, 1H), 2.23-2.13 (m, 1H), 1.65-1.56 (m, 2H).

### Example 245: trans-5-(2-(3-(Cyclopentyloxy)-4,5-difluorophenyl)cyclopropyl)-2,2′-bipyrimidine (single enantiomer I)

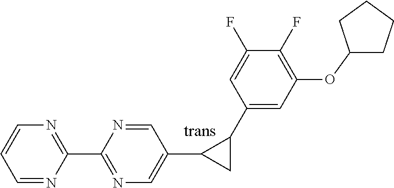 [SMILES: Fc1cc(C2CC2c2cnc(-c3ncccn3)nc2)cc(OC2CCCC2)c1F]

### Example 246: trans-5-(2-(3-(Cyclopentyloxy)-4,5-difluorophenyl)cyclopropyl)-2,2′-bipyrimidine (single enantiomer II)

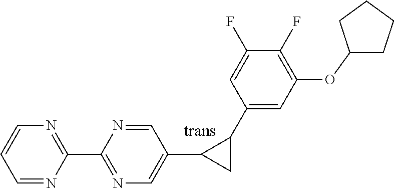 [SMILES: Fc1cc(C2CC2c2cnc(-c3ncccn3)nc2)cc(OC2CCCC2)c1F]

A mixture of enantiomers of trans-5-[2-[3-(cyclopentoxy)-4,5-difluoro-phenyl]cyclopropyl]-2-pyrimidin-2-yl-pyrimidine (180 mg) was separated by SFC (supercritical fluid chromatography) on a CHIRALCEL® OD column using liquid CO₂ and MeOH [0.1% aqueous NH₃ modifier] (40:60) to give trans-5-[2-[3-(cyclopentoxy)-4,5-difluoro-phenyl]cyclopropyl]-2-pyrimidin-2-yl-pyrimidine (single enantiomer I) as a white solid (faster eluting enantiomer, 72 mg, 39% yield, m/z: 395 [M+H]⁺ observed), and trans-5-[2-[3-(cyclopentoxy)-4,5-difluoro-phenyl]cyclopropyl]-2-pyrimidin-2-yl-pyrimidine(single enantiomer II) as a white solid (slower eluting enantiomer, 81 mg, 44% yield, m/z: 395 [M+H]⁺ observed).

### Example 245: trans-5-(2-(3-(Cyclopentyloxy)-4,5-difluorophenyl)cyclopropyl)-2,2′-bipyrimidine (single enantiomer I)

m/z: 395 [M+H]⁺ observed. ¹H NMR (400 MHz, DMSO-d₆): δ 8.99 (d, J=4.8 Hz, 2H), 8.84 (s, 2H), 7.62 (t, J=4.8 Hz, 1H), 6.90 (d, J=6.8 Hz, 1H), 6.87-6.82 (m, 1H), 4.97-4.94 (m, 1H), 2.39-2.38 (m, 1H), 1.95-1.92 (m, 2H), 1.76-1.68 (m, 7H), 1.59 (s, 2H).

### Example 246: trans-5-(2-(3-(Cyclopentyloxy)-4,5-difluorophenyl)cyclopropyl)-2,2′-bipyrimidine (single enantiomer II)

m/z: 395 [M+H]⁺ observed. ¹H NMR (400 MHz, DMSO-d₆): δ 8.99 (d, J=4.8 Hz, 2H), 8.84 (s, 2H), 7.62 (t, J=4.8 Hz, 1H), 6.90 (d, J=6.8 Hz, 1H), 6.87-6.82 (m, 1H), 4.97-4.94 (m, 1H), 2.39-2.38 (m, 1H), 1.95-1.92 (m, 2H), 1.76-1.68 (m, 7H), 1.59 (s, 2H).

### Example 247: trans-5-(2-(3,4-Difluoro-5-(((R)-tetrahydrofuran-3-yl)oxy)phenyl)cyclopropyl)-2,2′-bipyrimidine (single diastereomer I)

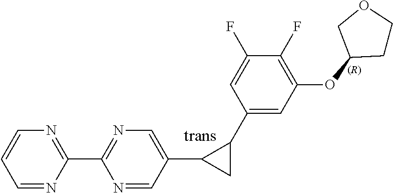

### Example 248: trans-5-(2-(3,4-Difluoro-5-(((R)-tetrahydrofuran-3-yl)oxy)phenyl)cyclopropyl)-2,2′-bipyrimidine (single diastereomer II)

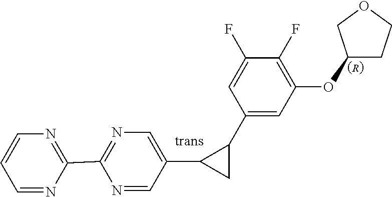

A mixture of diastereomers of trans-5-(2-(3,4-difluoro-5-((R)-tetrahydrofuran-3-yl)oxy)phenyl)cyclopropyl)-2,2′-bipyrimidine (280 mg) was separated by SFC (supercritical fluid chromatography) on a CHIRALPAK® IG column using liquid CO₂ and MeOH [0.1% aqueous NH₃ as modifier] (40:60) to give trans-5-(2-(3,4-difluoro-5-((R)-tetrahydrofuran-3-yl)oxy)phenyl)cyclopropyl)-2,2′-bipyrimidine (single diastereomer I) as a white solid (faster eluting diastereomer, 95 mg, 38% yield, m/z: 397 [M+H]⁺ observed) and trans-5-(2-(3,4-difluoro-5-((R)-tetrahydrofuran-3-yl)oxy)phenyl)cyclopropyl)-2,2′-bipyrimidine (single diastereomer II) as a white solid (slower eluting diastereomer, 113 mg, 45% yield, m/z: 397 [M+H]⁺ observed).

### Example 247: trans-5-(2-(3,4-Difluoro-5-(((R)-tetrahydrofuran-3-yl)oxy)phenyl)cyclopropyl)-2,2′-bipyrimidine (single diastereomer I)

m/z: 397 [M+H]⁺ observed. ¹H NMR (400 MHz, DMSO-d₆): δ 8.99 (d, J=4.8 Hz, 2H), 8.85 (s, 2H), 7.63 (t, J=4.8 Hz, 1H), 6.92-6.87 (m, 2H), 5.16-5.15 (m, 1H), 3.90-3.81 (m, 3H), 3.78-3.76 (m, 1H), 2.55-2.54 (m, 1H), 2.40-2.38 (m, 1H), 2.28-2.23 (m, 1H), 2.04-1.99 (m, 1H), 1.77-1.75 (m, 1H), 1.72-1.68 (m, 1H).

### Example 248: trans-5-(2-(3,4-Difluoro-5-(((R)-tetrahydrofuran-3-yl)oxy)phenyl)cyclopropyl)-2,2′-bipyrimidine (single diastereomer II)

m/z: 397 [M+H]⁺ observed. ¹H NMR (400 MHz, DMSO-d₆): δ 8.99 (d, J=4.8 Hz, 2H), 8.85 (s, 2H), 7.63 (t, J=4.8 Hz, 1H), 6.92-6.87 (m, 2H), 5.16-5.15 (m, 1H), 3.90-3.81 (m, 3H), 3.78-3.76 (m, 1H), 2.55-2.54 (m, 1H), 2.40-2.38 (m, 1H), 2.28-2.23 (m, 1H), 2.04-1.99 (m, 1H), 1.77-1.75 (m, 1H), 1.72-1.68 (m, 1H).

### Example 249: trans-5-(2-(3,4-Difluoro-5-(((S)-tetrahydrofuran-3-yl)oxy)phenyl)cyclopropyl)-2,2′-bipyrimidine (single diastereomer I)

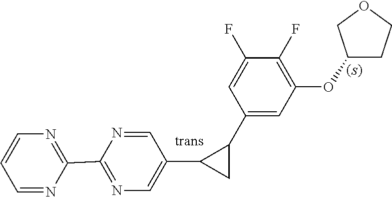

### Example 250: trans-5-(2-(3,4-Difluoro-5-(((S)-tetrahydrofuran-3-yl)oxy)phenyl)cyclopropyl)-2,2′-bipyrimidine (single diastereomer II)

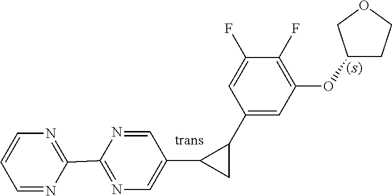

A mixture of diastereomers (350 mg) was separated by SFC (supercritical fluid chromatography) on a CHIRALCEL® OD column using liquid CO₂ and MeOH [0.1% aqueous NH₃ as modifier] (40:60) to give trans-5-(2-(3,4-difluoro-5-((S)-tetrahydrofuran-3-yl)oxy)phenyl)cyclopropyl)-2,2′-bipyrimidine (single diastereomer I) as a white solid (faster eluting diastereomer, 106 mg, 30% yield, m/z: 397 [M+H]⁺), and trans-5-(2-(3,4-difluoro-5-(((S)-tetrahydrofuran-3-yl)oxy)phenyl)cyclopropyl)-2,2′-bipyrimidine (single diastereomer II) as a white solid (slower eluting diastereomer, 135 mg, 38% yield, m/z: 397 [M+H]⁺).

### Example 249: trans-5-(2-(3,4-Difluoro-5-(((S)-tetrahydrofuran-3-yl)oxy)phenyl)cyclopropyl)-2,2′-bipyrimidine (single diastereomer I)

m/z: 397 [M+H]⁺. ¹H NMR (400 MHz, DMSO-d₆): δ 8.99 (d, J=4.8 Hz, 2H), 8.85 (s, 2H), 7.63 (t, J=4.8 Hz, 1H), 6.92-6.87 (m, 2H), 5.16 (t, J=4.8 Hz, 1H), 3.91-3.81 (m, 3H), 3.78-3.76 (m, 1H), 2.55-2.54 (m, 1H), 2.41-2.38 (m, 1H), 2.28-2.23 (m, 1H), 2.03-1.96 (m, 1H), 1.77-1.73 (m, 1H), 1.70-1.68 (m, 1H).

### Example 250: trans-5-(2-(3,4-Difluoro-5-(((S)-tetrahydrofuran-3-yl)oxy)phenyl)cyclopropyl)-2,2′-bipyrimidine (single diastereomer II)

m/z: 397 [M+H]⁺. ¹H NMR (400 MHz, DMSO-d₆): δ 8.99 (d, J=4.8 Hz, 2H), 8.85 (s, 2H), 7.63 (t, J=4.8 Hz, 1H), 6.92-6.87 (m, 2H), 5.16 (t, J=4.8 Hz, 1H), 3.91-3.81 (m, 3H), 3.78-3.76 (m, 1H), 2.55-2.54 (m, 1H), 2.41-2.38 (m, 1H), 2.28-2.23 (m, 1H), 2.03-1.96 (m, 1H), 1.77-1.73 (m, 1H), 1.70-1.68 (m, 1H).

### Example 251: trans-5-(2-(3,4-Difluoro-5-(4-methoxy-1H-pyrazol-1-yl)phenyl)cyclopropyl)-2,2′-bipyrimidine (single enantiomer I)

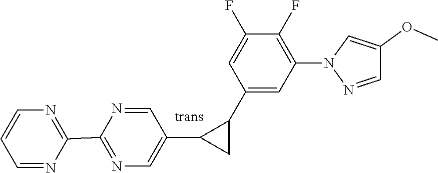 [SMILES: COc1cnn(-c2cc(C3CC3c3cnc(-c4ncccn4)nc3)cc(F)c2F)c1]

### Example 252: trans-5-(2-(3,4-Difluoro-5-(4-methoxy-1H-pyrazol-1-yl)phenyl)cyclopropyl)-2,2′-bipyrimidine (single enantiomer II)

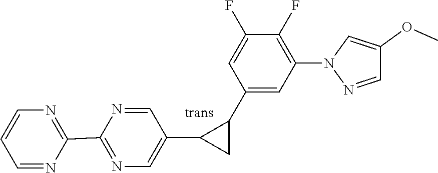 [SMILES: COc1cnn(-c2cc(C3CC3c3cnc(-c4ncccn4)nc3)cc(F)c2F)c1]

### 1-(5-Bromo-2,3-fluorophenyl)-4-methoxy-1H-pyrazole

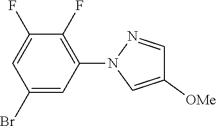 [SMILES: COc1cnn(-c2cc(Br)cc(F)c2F)c1]

To a solution of 5-bromo-1,2,3-trifluorobenzene (500.0 mg, 2.38 mmol) in DMSO (5 mL) was added K₂CO₃ (490 mg, 3.55 mmol) and 4-methoxy-1H-pyrazole (0.94 g, 9.6 mmol). The reaction mixture was stirred at 50° C. for 6 h. The mixture was cooled to rt, diluted with water (100 mL) and extracted with EtOAc (2×100 mL). The organic layer was washed with saturated aqueous brine solution (100 mL), dried over Na₂SO₄ and evaporated. The crude was purified by normal phase SiO₂ chromatography (0-3% EtOAc/petroleum ether) to afford 1-(5-bromo-2,3-difluorophenyl)-4-methoxy-1H-pyrazole as an off-white solid (0.50 g, 72% yield, m/z: 289 [M+H]⁺ observed. ¹H NMR (400 MHz, CDCl₃): δ 7.91-7.92 (m, 1 H), 7.67-7.66 (m, 1 H), 7.52 (s, 1 H), 7.26-7.20 (m, 1 H), 3.82 (s, 3H).

### (E)-1-(2,3-Difluoro-5-(2-(4,4,5,5-tetramethyl-1,3,2-dioxaborolan-2-yl)vinyl)phenyl)-4-methoxy-1H-pyrazole:

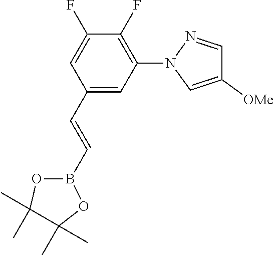 [SMILES: COc1cnn(-c2cc(/C=C/B3OC(C)(C)C(C)(C)O3)cc(F)c2F)c1]

To a solution of 1-(5-bromo-2,3-difluorophenyl)-4-methoxy-1H-pyrazole (1.5 g, 5.2 mmol) in toluene (25 mL) was added 4,4,5,5-tetramethyl-2-vinyl-1,3,2-dioxaborolane (1.20 g, 7.78 mmol) and TEA (4.2 mL, 30 mmol) at room temperature. The reaction mixture purged with N₂ gas for 10 min, then Pd(t-Bu3P)2 (53 mg, 0.10 mmol) was added and the degassing continued for 10 min. The reaction mixture was heated to 120° C. for 16 h in a sealed tube. The reaction mixture was cooled to rt, diluted with EtOAc (250 mL), filtered through a CELITE® pad and the filtrate was evaporated to afford (E) -1-(2,3-difluoro-5-(2-(4,4,5,5-tetramethyl-1,3,2-dioxaborolan-2-yl)vinyl)phenyl)-4-methoxy-1H-pyrazole as a pale yellow resin (1.6 g, 85% yield, m/z: 363 [M+H]⁺observed), which was used in the next step without further purification.

### trans-1-(2,3-Difluoro-5-(2-(4,4,5,5-tetramethyl-1,3,2-dioxaborolan-2-yl)cyclopropyl)phenyl)-4-methoxy-1H-pyrazole

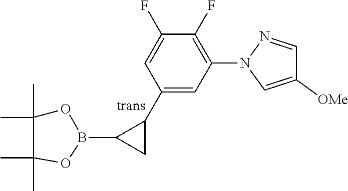 [SMILES: COc1cnn(-c2cc(C3CC3B3OC(C)(C)C(C)(C)O3)cc(F)c2F)c1]

To a solution (E)-1-(2,3-difluoro-5-(2-(4,4,5,5-tetramethyl-1,3,2-dioxaborolan-2-yl)vinyl)phenyl)-4-methoxy-1H-pyrazole (2.0 g, 5.5 mmol) in THF (15 mL) at 0° C. was added Pd(OAc)₂ (trimer, 0.74 g, 0.11 mmol) and freshly prepared ethereal diazomethane [prepared from N-methyl-N-nitroso urea (13.9 g, 115 mmol) in KOH (50% solution in H₂O, 100 mL) and Et₂O (100 mL) at 0° C.]. The reaction mixture was stirred at 0° C. for 5 min, then kept in a refrigerator for 16 h. The reaction mixture was filtered through a CELITE® pad and filtrate was evaporated to dryness and the process was repeated twice to afford 1-(2,3-difluoro-5-(2-(4,4,5,5-tetramethyl-1,3,2-dioxaborolan-2-yl)cyclopropyl)phenyl)-4-methoxy-1H-pyrazole as a yellow resin (2.0 g, 96% yield, m/z: 377 [M+H]^{P}observed), which was used in the next step without further purification.

### trans-2-Chloro-5-(2-(3,4-difluoro-5-(4-methoxy-1H-pyrazol-1-yl)phenyl)cyclopropyl)pyrimidine

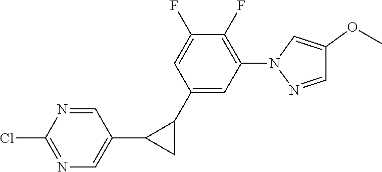 [SMILES: COc1cnn(-c2cc(C3CC3c3cnc(Cl)nc3)cc(F)c2F)c1]

To a solution of 1-(2,3-difluoro-5-(2-(4,4,5,5-tetramethyl-1,3,2-dioxaborolan-2-yl)cyclopropyl)phenyl)-4-methoxy-1H-pyrazole (1.0 g, 4.0 mmol) in MeOH-H₂O (1:1, 20 mL) at 0° C. was added KHF₂ (1.55 g, 19.9 mmol). The reaction mixture was stirred at 90° C. for 16 h. The mixture was filtered through CELITE® pad and the filtrate was evaporated to dryness. The residue was redissolved in MeOH and the mixture was evaporated to dryness to afford 1-(2,3-difluoro-5-(2-(trifluoro-λ⁴-boraneyl)cyclopropyl)phenyl)-4-methoxy-1H-pyrazole, potassium salt as a yellow resin (1.0 g, 71% yield), which was used in the next step without further purification.

To a solution of crude 1-(2,3-difluoro-5-(2-(trifluoro-λ⁴-boraneyl)cyclopropyl)phenyl)-4-methoxy-1H-pyrazole, potassium salt (0.70 g, 2.0 mmol) in 1,4-dioxane-water (5:1, 54 mL) was added 5-bromo-2-chloropyrimidine (0.52 g, 2.66 mmol) and Cs₂CO₃ (2.17 g, 6.66 mmol) at room temperature. The mixture was purged with N₂ gas for 10 min, then Pd(PPh3)4 (109.0 mg, 0.090 mmol) was added and the degassing was continued for 10 min. The reaction mixture was heated at 100° C. for 16 h in a sealed tube. The reaction mixture was cooled to rt, diluted with EtOAc (500 mL), filtered through a CELITE® pad and the filtrate was evaporated to afford 2-chloro-5-(2-(3,4-difluoro-5-(4-methoxy-1H-pyrazol-1-yl)phenyl)cyclopropyl)pyrimidine as an off-white solid (0.40 g, 56% yield, m/z: 363 [M+H]⁺ observed), which was used in the next step without further purification.

### trans-5-(2-(3,4-Difluoro-5-(4-methoxy-1H-pyrazol-1-yl)phenyl)cyclopropyl)-2,2′-bipyrimidine

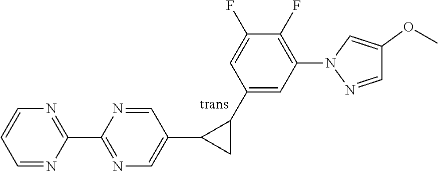 [SMILES: COc1cnn(-c2cc(C3CC3c3cnc(-c4ncccn4)nc3)cc(F)c2F)c1]

To a solution 2-chloro-5-(2-(3,4-difluoro-5-(4-methoxy-1H-pyrazol-1-yl)phenyl)cyclopropyl)pyrimidine (0.30 g, 0.83 mmol) in DMF (5 mL) was added 2-(tributylstannyl)pyrimidine (0.26 mL, 0.83 mmol), tetraethylammonium chloride (0.14 g, 0.83 mmol) and K₂CO₃ (0.23 g, 1.66 mmol) at room temperature. The mixture was purged with N₂ gas for 10 min, then PdCl₂(PPh₃)₂ (58 mg, 0.083 mmol) was added and the degassing was continued for 10 min. The reaction mixture was stirred at 110° C. for 16 h in a sealed tube. The reaction mixture was cooled to rt, diluted with water (50 mL) and extracted with EtOAc (2×50 mL). The organic layer was washed with saturated aqueous brine solution (50 mL), dried over Na₂SO₄ and evaporated to dryness. The residue was purified by reverse phase HPLC to afford trans-5-(2-(3,4-difluoro-5-(4-methoxy-1H-pyrazol-1-yl)phenyl)cyclopropyl)-2,2′-bipyrimidine (170 mg, 50% yield, m/z: 407 [M+H]⁺ observed). A mixture of enantiomers of trans-5-(2-(3,4-difluoro-5-(4-methoxy-1H-pyrazol-1-yl)phenyl)cyclopropyl)-2,2′-bipyrimidine (150 mg) was separated by SFC (supercritical fluid chromatography) on a CHIRALCEL® OD-H column using liquid CO₂ and MeOH (60:40) to give trans-5-(2-(3,4-difluoro-5-(4-methoxy-1H-pyrazol-1-yl)phenyl)cyclopropyl)-2,2′-bipyrimidine (single enantiomer I) as an off-white solid (faster eluting enantiomer, 21 mg, 14%, m/z: 407 [M+H]⁺ observed), and trans-5-(2-(3,4-difluoro-5-(4-methoxy-1H-pyrazol-1-yl)phenyl)cyclopropyl)-2,2′-bipyrimidine (single enantiomer II) as an off-white solid (slower eluting enantiomer, 20 mg, 14%, m/z: 407 [M+H]⁺ observed).

### Example 251: trans-5-(2-(3,4-Difluoro-5-(4-methoxy-1H-pyrazol-1-yl)phenyl)cyclopropyl)-2,2′-bipyrimidine (single enantiomer I)

m/z: 407 [M+H]⁺ observed. ¹H NMR (400 MHz, DMSO-d₆): δ 8.99 (d, 2H), 8.86 (s, 2 H), 8.01 (d, 1H), 7.66 (s, 1H), 7.62 (t, 1H), 7.35-7.33 (m, 1H), 7.33-7.30 (m, 1H), 3.71 (s, 3H), 2.67-2.63 (m, 1H), 2.51-2.37 (m, 1H), 1.81-1.72 (m, 2H).

### Example 252: trans-5-(2-(3,4-Difluoro-5-(4-methoxy-1H-pyrazol-1-yl)phenyl)cyclopropyl)-2,2′-bipyrimidine (single enantiomer II)

m/z: 407 [M+H]⁺ observed. ¹H NMR (400 MHz, DMSO-d₆): δ 8.99 (d, 2H), 8.86 (s, 2 H), 8.01 (d, 1H), 7.66 (s, 1H), 7.62 (t, 1H), 7.35-7.33 (m, 1H), 7.33-7.30 (m, 1H), 3.71 (s, 3H), 2.67-2.63 (m, 1H), 2.51-2.37 (m, 1H), 1.81-1.72 (m, 2H).

The following examples were prepared in a similar manner as trans-5-(2-(3,4-difluoro-5-(4-methoxy-1H-pyrazol-1-yl)phenyl)cyclopropyl)-2,2′-bipyrimidine from 5-bromo-1,2,3-trifluorobenzene and an appropriate amine.

### Example 253: trans-5-(2-(3,4-difluoro-5-(1H-pyrazol-1-yl)phenyl)cyclopropyl)-2,2′-bipyrimidine (single enantiomer I)

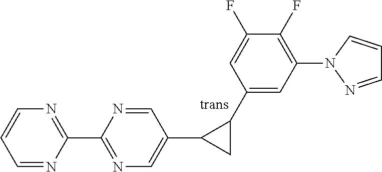 [SMILES: Fc1cc(C2CC2c2cnc(-c3ncccn3)nc2)cc(-n2cccn2)c1F]

### Example 254: trans-5-(2-(3,4-Difluoro-5-(1H-pyrazol-1-yl)phenyl)cyclopropyl)-2,2′-bipyrimidine (single enantiomer II)

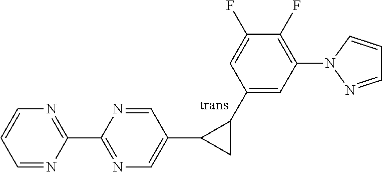 [SMILES: Fc1cc(C2CC2c2cnc(-c3ncccn3)nc2)cc(-n2cccn2)c1F]

A mixture of enantiomers of trans-5-(2-(3,4-difluoro-5-(1H-pyrazol-1-yl)phenyl)cyclopropyl)-2,2′-bipyrimidine (150 mg) was separated by SFC (supercritical fluid chromatography) on a CHIRALPAK® AD-H column using liquid CO₂ and 30 mM ammonia in MeOH (55:45) to give trans-5-(2-(3,4-difluoro-5-(1H-pyrazol-1-yl)phenyl)cyclopropyl)-2,2′-bipyrimidine (single enantiomer I) as an off-white solid (faster eluting enantiomer, 38 mg, 25%, m/z: 377 [M+H]⁺ observed), and trans-5-(2-(3,4-difluoro-5-(1H-pyrazol-1-yl)phenyl)cyclopropyl)-2,2′-bipyrimidine (single enantiomer II) as an off-white solid (slower eluting enantiomer, 36 mg, 24%, m/z: 377 [M+H]⁺ observed).

### Example 253: trans-5-(2-(3,4-Difluoro-5-(1H-pyrazol-1-yl)phenyl)cyclopropyl)-2,2′-bipyrimidine (single enantiomer I)

m/z: 377 [M+H]⁺ observed. ¹H NMR (400 MHz, DMSO-d₆): δ 8.99 (d, 2H), 8.86 (s, 2H), 8.27 (t, 1H), 7.84 (d, 1H), 7.63 (t, 1H), 7.56-7.51 (m, 1H), 7.43-7.34 (m, 1H), 6.64-6.57 (m, 1H), 2.71-2.64 (m, 1H), 2.49-2.43 (m, 1H), 1.85-1.69 (m, 2H).

### Example 254: trans-5-(2-(3,4-Difluoro-5-(1H-pyrazol-1-yl)phenyl)cyclopropyl)-2,2′-bipyrimidine (single enantiomer II)

m/z: 377 [M+H]⁺ observed. ¹H NMR (400 MHz, DMSO-d₆): δ 8.99 (d, 2H), 8.86 (s, 2H), 8.27 (t, 1H), 7.84 (d, 1H), 7.63 (t, 1H), 7.56-7.51 (m, 1H), 7.43-7.34 (m, 1H), 6.64-6.57 (m, 1H), 2.71-2.64 (m, 1H), 2.49-2.43 (m, 1H), 1.85-1.69 (m, 2H).

### Example 255: trans-5-(2-(3,4-Difluoro-5-(3-phenylazetidin-1-yl)phenyl)cyclopropyl)-2,2′-bipyrimidine (single enantiomer I)

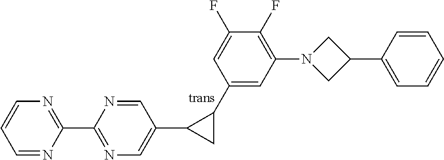 [SMILES: Fc1cc(C2CC2c2cnc(-c3ncccn3)nc2)cc(N2CC(c3ccccc3)C2)c1F]

### Example 256: trans-5-(2-(3,4-Difluoro-5-(3-phenylazetidin-1-yl)phenyl)cyclopropyl)-2,2′-bipyrimidine (single enantiomer II)

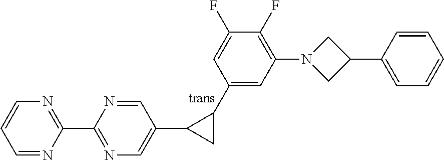 [SMILES: Fc1cc(C2CC2c2cnc(-c3ncccn3)nc2)cc(N2CC(c3ccccc3)C2)c1F]

A mixture of enantiomers of trans-5-(2-(3,4-difluoro-5-(3-phenylazetidin-1-yl)phenyl)cyclopropyl)-2,2′-bipyrimidine (100 mg) was separated by SFC (supercritical fluid chromatography) on a CHIRALCEL® OD-3 column using liquid CO₂ and MeOH-MeCN [1:1] (60:40) to give trans-5-(2-(3,4-difluoro-5-(3-phenylazetidin-1-yl)phenyl)cyclopropyl)-2,2′-bipyrimidine (single enantiomer I) as an off-white solid (faster eluting enantiomer, 20 mg, 20%, m/z: 442 [M+H]⁺ observed), and trans-5-(2-(3,4-difluoro-5-(3-phenylazetidin-1-yl)phenyl)cyclopropyl)-2,2′-bipyrimidine (single enantiomer II) as an off-white solid (slower eluting enantiomer, 25 mg, 25%, m/z: 442 [M+H]⁺ observed).

### Example 255: trans-5-(2-(3,4-Difluoro-5-(3-phenylazetidin-1-yl)phenyl)cyclopropyl)-2,2′-bipyrimidine (single enantiomer I)

m/z: 442 [M+H]⁺ observed. ¹H NMR (400 MHz, DMSO-d₆): δ 8.98 (d, 2H), 8.83 (s, 2H), 7.62 (t, 1H), 7.42-7.34 (m, 4H), 7.26-7.23 (m, 1H), 6.63-6.59 (m, 1H), 6.62 (d, 1H), 4.39 (br s, 2H), 4.00-3.93 (m, 3H), 2.50-2.32 (m, 2H), 1.73-1.62 (m, 2H).

### Example 256: trans-5-(2-(3,4-Difluoro-5-(3-phenylazetidin-1-yl)phenyl)cyclopropyl)-2,2′-bipyrimidine (single enantiomer II)

m/z: 442 [M+H]⁺ observed. ¹H NMR (400 MHz, DMSO-d₆): δ 8.98 (d, 2H), 8.83 (s, 2H), 7.62 (t, 1H), 7.42-7.34 (m, 4H), 7.26-7.23 (m, 1H), 6.63-6.59 (m, 1H), 6.62 (d, 1H), 4.39 (br s, 2H), 4.00-3.93 (m, 3H), 2.50-2.32 (m, 2H), 1.73-1.62 (m, 2H).

### Example 257: trans-5-(2-(3-(3,4-Difluoro-1H-pyrrol-1-yl)-4,5-difluorophenyl)cyclopropyl)-2,2′-bipyrimidine (single enantiomer I)

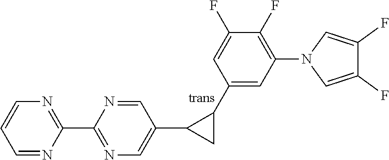 [SMILES: Fc1cn(-c2cc(C3CC3c3cnc(-c4ncccn4)nc3)cc(F)c2F)cc1F]

### Example 258: trans-5-(2-(3-(3,4-Difluoro-1H-pyrrol-1-yl)-4,5-difluorophenyl)cyclopropyl)-2,2′-bipyrimidine (single enantiomer II)

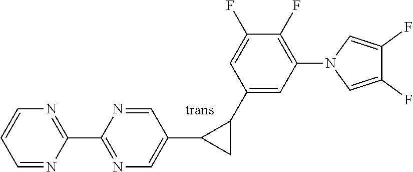 [SMILES: Fc1cn(-c2cc(C3CC3c3cnc(-c4ncccn4)nc3)cc(F)c2F)cc1F]

A mixture of enantiomers of trans-5-(2-(3-(3,4-difluoro-1H-pyrrol-1-yl)-4,5-difluorophenyl)cyclopropyl)-2,2′-bipyrimidine (120 mg) was separated by SFC (supercritical fluid chromatography) on a CHIRALPAK® OD-3 column using liquid CO₂ and MeOH (60:40) to give trans-5-(2-(3-(3,4-difluoro-1H-pyrrol -1-yl)-4,5-difluorophenyl)cyclopropyl)-2,2′-bipyrimidine (single enantiomer I) as an off-white solid (faster eluting enantiomer, 38 mg, 32% yield, m/z: 412 [M+H]⁺ observed), and trans-5-(2-(3-(3,4-difluoro-1H-pyrrol-1-yl)-4,5-difluorophenyl)cyclopropyl)-2,2′-bipyrimidine (single enantiomer II) as an off-white solid (slower eluting enantiomer, 39 mg, 32% yield, m/z: 412 [M+H]⁺ observed).

### Example 257: trans-5-(2-(3-(3,4-Difluoro-1H-pyrrol-1-yl)-4,5-difluorophenyl)cyclopropyl)-2,2′-bipyrimidine (single enantiomer I)

m/z: 412 [M+H]⁺ observed. ¹H NMR (400 MHz, DMSO-d₆): δ 8.99 (d, 2H), 8.85 (s, 2H), 7.62 (t, 1H), 7.39-7.36 (m, 1H), 7.34 (d, 2H), 7.25 (d, 1H), 2.58-2.54 (m, 1H), 2.50-2.46 (m, 1H),1.83-1.75 (m, 2H).

### Example 258: trans-5-(2-(3-(3,4-Difluoro-1H-pyrrol-1-yl)-4,5-difluorophenyl)cyclopropyl)-2,2′-bipyrimidine (single enantiomer II)

m/z: 412 [M+H]⁺ observed. ¹H NMR (400 MHz, DMSO-d₆): δ 8.99 (d, 2H), 8.85 (s, 2H), 7.62 (t, 1H), 7.39-7.36 (m, 1H), 7.34 (d, 2H), 7.25 (d, 1H), 2.58-2.54 (m, 1H), 2.50-2.46 (m, 1H),1.83-1.75 (m, 2H).

### Example 259: trans-1-(5-(2-([2,2′-Bipyrimidin]-5-yl)cyclopropyl)-2,3-difluorophenyl)-1H-pyrazol-4-ol (single enantiomer I)

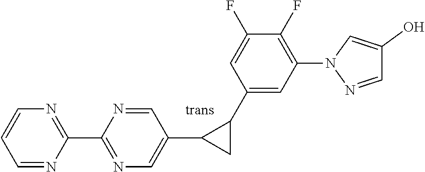 [SMILES: Oc1cnn(-c2cc(C3CC3c3cnc(-c4ncccn4)nc3)cc(F)c2F)c1]

### Example 260: trans-1-(5-(2-([2,2′-Bipyrimidin]-5-yl)cyclopropyl)-2,3-difluorophenyl)-1H-pyrazol-4-ol (single enantiomer II)

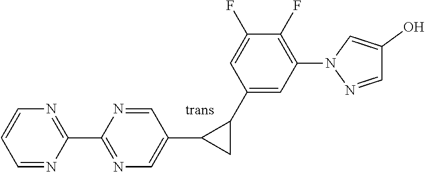 [SMILES: Oc1cnn(-c2cc(C3CC3c3cnc(-c4ncccn4)nc3)cc(F)c2F)c1]

To a solution of trans-5-(2-(3,4-difluoro-5-(4-methoxy-1H-pyrazol-1-yl)phenyl)cyclopropyl)-2,2′-bipyrimidine (single enantiomer I, faster eluting enantiomer) (20.0 mg, 0.049 mmol) in CH₂Cl₂ (3 mL) was added BBr₃ (0.31g, 0.20 mmol) was added at 0° C. The reaction mixture stirred at room temperature for 16 h. The reaction mixture was quenched with MeOH (0.5 mL), diluted with water (10 mL) and extracted with CH₂Cl₂-MeOH (9:1, 2×30 mL). The organic layer was washed with saturated aqueous NaHCO₃ solution (15 mL), saturated aqueous brine solution (20 mL), dried over Na₂SO₄ and evaporated to dryness. The residue was triturated with diethyl ether and dried to afford trans-1-(5-(2-([2,2′-bipyrimidin]-5-yl)cyclopropyl)-2,3-difluorophenyl)-1H-pyrazol-4-ol as an off-white solid (12 mg, 62% yield, m/z: 393 [M+H]⁺ observed).

The reaction conditions above were utilized to prepare trans-1-(5-(2-([2,2′-bipyrimidin]-5-yl)cyclopropyl)-2,3-difluorophenyl)-1H-pyrazol-4-ol as an off-white solid, enantiomer II, (16 mg, 80% yield, m/z: 393 [M+H]⁺ observed, from trans-5-(2-(3,4-difluoro-5-(4-methoxy-1H-pyrazol-1-yl)phenyl)cyclopropyl)-2,2′-bipyrimidine (single enantiomer II, slower eluting enantiomer) (20.0 mg, 0.049 mmol).

### Example 259: trans-1-(5-(2-([2,2′-Bipyrimidin]-5-yl)cyclopropyl)-2,3-difluorophenyl)-1H-pyrazol-4-ol (single enantiomer I)

m/z: 393 [M+H]⁺ observed. ¹H NMR (400 MHz, DMSO-d₆): δ 8.99 (br s, 3H), 8.86 (br s, 2H), 7.71 (d, 1H), 7.64-7.62 (m, 1H), 7.50-7.46 (m, 2H), 7.31-7.26 (m, 1H), 2.67-2.62 (m, 1H), 2.51-2.40 (m, 1H), 1.81-1.75 (m, 1H), 1.73-1.68 (m, 1H).

### Example 260: trans-1-(5-(2-([2,2′-Bipyrimidin]-5-yl)cyclopropyl)-2,3-difluorophenyl)-1H-pyrazol-4-ol (single enantiomer II)

m/z: 393 [M+H]⁺ observed. ¹H NMR (400 MHz, DMSO-d₆): δ 8.99 (br s, 3H), 8.86 (br s, 2H), 7.71 (d, 1H), 7.64-7.62 (m, 1H), 7.50-7.46 (m, 2H), 7.31-7.26 (m, 1H), 2.67-2.62 (m, 1H), 2.51-2.40 (m, 1H), 1.81-1.75 (m, 1H), 1.73-1.68 (m, 1H).

### Example 261: trans-5-(2-(3,4-Difluoro-5-(4-methyl-1H-imidazol-1-yl)phenyl)cyclopropyl)-2,2′-bipyrimidine (single enantiomer I)

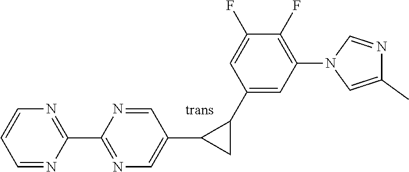 [SMILES: Cc1cn(-c2cc(C3CC3c3cnc(-c4ncccn4)nc3)cc(F)c2F)cn1]

### Example 262: trans-5-(2-(3,4-Difluoro-5-(4-methyl-1H-imidazol-1-yl)phenyl)cyclopropyl)-2,2′-bipyrimidine (single enantiomer II)

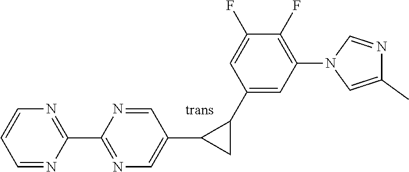 [SMILES: Cc1cn(-c2cc(C3CC3c3cnc(-c4ncccn4)nc3)cc(F)c2F)cn1]

A mixture of enantiomers of trans-5-(2-(3,4-difluoro-5-(4-methyl-1H-imidazol-1-yl)phenyl)cyclopropyl)-2,2′-bipyrimidine (100 mg) was separated by SFC (supercritical fluid chromatography) on a CHIRALCEL® OJ-H column using liquid CO₂ and 7N ammonia in MeOH (80:20) to give trans-5-(2-(3,4-difluoro-5-(4-methyl-1H-imidazol-1-yl)phenyl)cyclopropyl)-2,2′-bipyrimidine (single enantiomer I) as an off-white solid (faster eluting enantiomer, 30 mg, 30%, m/z: 391 [M+H]⁺ observed), and trans-5-(2-(3,4-difluoro-5-(4-methyl-1H-imidazol-1-yl)phenyl)cyclopropyl)-2,2′-bipyrimidine (single enantiomer II) as an off-white solid (slower eluting enantiomer, 25 mg, 25%, m/z: 391 [M+H]⁺ observed).

### Example 261: trans-5-(2-(3,4-Difluoro-5-(4-methyl-1H-imidazol-1-yl)phenyl)cyclopropyl)-2,2′-bipyrimidine (single enantiomer I)

m/z: 391 [M+H]⁺ observed. ¹H NMR (400 MHz, DMSO-d₆): δ 8.99 (d, 2H), 8.85 (s, 2H), 7.98 (s, 1H) 7.62 (t, 1H), 7.43-7.38 (m, 1H), 7.35-7.33 (m, 2H), 2.59-2.55 (m, 1H), 2.50-2.46 (m, 1H), 2.11 (s, 3H), 1.82-1.75 (m, 2H).

### Example 262: trans-5-(2-(3,4-Difluoro-5-(4-methyl-1H-imidazol-1-yl)phenyl)cyclopropyl)-2,2′-bipyrimidine (single enantiomer II)

m/z: 391 [M+H]⁺ observed. ¹H NMR (400 MHz, DMSO-d₆): δ 8.99 (d, 2H), 8.85 (s, 2H), 7.98 (s, 1H) 7.62 (t, 1H), 7.43-7.38 (m, 1H), 7.35-7.33 (m, 2H), 2.59-2.55 (m, 1H), 2.50-2.46 (m, 1H), 2.11 (s, 3H), 1.82-1.75 (m, 2H).

### Example 263: trans-Ethyl 2-([2,2′-bipyrimidin]-5-yl)-3-(3,4-difluoro-5-methoxyphenyl)cyclopropane-1-carboxylate

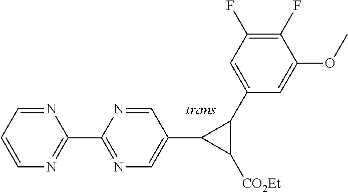 [SMILES: CCOC(=O)C1C(c2cnc(-c3ncccn3)nc2)C1c1cc(F)c(F)c(OC)c1]

### (E)-2-Chloro-5-(2-(4,4,5,5-tetramethyl-1,3,2-dioxaborolan-2-yl)vinyl)pyrimidine

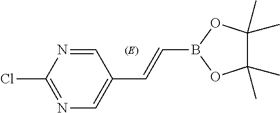 [SMILES: CC1(C)OB(/C=C/c2cnc(Cl)nc2)OC1(C)C]

A dry, 100 mL round bottom flask equipped with a stir bar, reflux condenser and gas inlet adapter was charged with 4,4,5,5-tetramethyl-2-vinyl-1,3,2-dioxaborolane (2.19 g, 14.2 mmol), 5-bromo-2-chloropyrimidine (2.5 g, 12.9 mmol), tri-t-butylphosphonium tetrafluoroborate (750 mg, 2.58 mmol), tris(dibenzylideneacetone)dipalladium(0) (1.18 g, 1.29 mmol) and dry, degassed toluene (40 mL), followed by DIPEA (4.5 mL, 25.9 mmol). The reaction mixture was purged with nitrogen gas for 5 minutes. The mixture was heated at 95° C. for 4 hours under nitrogen, then cooled to rt. The volatiles were evaporated, to the residue was added water (50 mL) and the mixture extracted with EtOAc (3×50 mL). The combined organic phase was dried over Na₂SO₄, filtered and evaporated. The residue was purified via normal phase SiO₂ chromatography (10% EtOAc/Hexanes). The desired fractions were collected and evaporated to give (E)-2-chloro-5-(2-(4,4,5,5-tetramethyl-1,3,2-dioxaborolan-2-yl)vinyl)pyrimidine as a pale yellow solid (1.1 g, 32% yield, m/z: 267 [M+H]⁺ observed). ¹H NMR (400 MHz, CDCl₃): δ 8.69 (s, 2H), 7.26 (d, J=18.5 Hz, 1H), 6.33 (d, J=18.6 Hz, 1H), 1.32 (s, 12H).

### trans-Ethyl 2-(2-chloropyrimidin-5-yl)-3-(4,4,5,5-tetramethyl-1,3,2-dioxaborolan-2-yl)cyclopropane-1-carboxylate

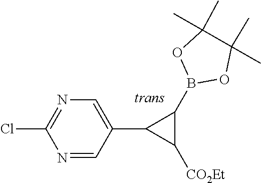 [SMILES: CCOC(=O)C1C(B2OC(C)(C)C(C)(C)O2)C1c1cnc(Cl)nc1]

To a dry microwave vial equipped with a stir bar was added (E)-2-chloro-5-(2-(4,4,5,5-tetramethyl-1,3,2-dioxaborolan-2-yl)vinyl)pyrimidine (250 mg, 0.94 mmol) and palladium(II) acetate (21 mg, 0.09 mmol), followed by dry THF (1 mL). The reaction mixture was purged with nitrogen gas for 5 minutes. The mixture was cooled to 0-5° C., then ethyl 2-diazoacetate (15 wt % solution in toluene, 1.3 mL, 1.88 mmol) was added dropwise over 1 hour. After 6 hours, additional ethyl 2-diazoacetate (15 wt % solution in toluene, 1.3 mL, 1.88 mmol) was added dropwise over 1 hour to the reaction mixture at rt. The mixture was stirred at rt under nitrogen overnight. The reaction mixture was evaporated to give an orange viscous oil. The residue was purified via normal phase SiO₂ chromatography (0-30% EtOAc/Hexanes). The desired fractions were collected and evaporated to give trans-ethyl 2-(2-chloropyrimidin-5-yl)-3-(4,4,5,5-tetramethyl-1,3 ,2-dioxaborolan-2-yl)cyclopropane-1-carboxylate as a pale yellow viscous oil (157 mg, 47% yield, m/z: 353 [M+H]⁺ observed), which was used in the next step without further purification.

### trans-Ethyl 2-(2-chloropyrimidin-5-yl)-3-(3,4-difluoro-5-methoxyphenyl)cyclopropane-1-carboxylate

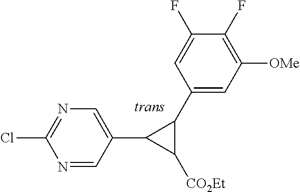 [SMILES: CCOC(=O)C1C(c2cnc(Cl)nc2)C1c1cc(F)c(F)c(OC)c1]

A microwave vial equipped with a stir bar was charged with 5-bromo-1,2-difluoro-3-methoxy-benzene (116 mg , 0.52 mmol), crude trans-ethyl 2-(2-chloropyrimidin-5-yl)-3-(4,4,5,5-tetramethyl-1,3,2-dioxaborolan-2-yl)cyclopropanecarboxylate (166 mg , 0.47 mmol), [1,1′-bis(diphenylphosphino)ferrocene]dichloropalladium(II), 1:1 complex with dichloromethane, (39 mg, 0.05 mmol), cesium carbonate (461 mg, 1.41 mmol) and THF-Water (9:1, 10 mL). The mixture was purged with nitrogen gas for 5 minutes. The vial was sealed and heated thermally at 80° C. for 4 hours behind a blast shield. The reaction mixture was cooled to rt, diluted with CH₂Cl₂ (20 mL) and filtered through a plug of CELITE®, rinsed and evaporated to a dark brown viscous oil. The residue was purified via normal phase SiO₂ chromatography (0-30% EtOAc/Hexanes). The desired fractions were collected and evaporated under reduced pressure to give trans-ethyl 2-(2-chloropyrimidin-5-yl)-3-(3,4-difluoro-5-methoxy-phenyl)cyclopropanecarboxylate as a pale yellow resin (25 mg, 14% yield, m/z: 269 [M+H]⁺ observed). ¹H NMR (400 MHz, CDCl₃): δ 8.59 (s, 2H), 6.68-6.55 (m, 2H), 4.12-3.99 (m, 2H), 3.93 (s, 3H), 3.14 (dd, J=6.7, 5.5 Hz, 1H), 2.72 (dd, J=9.3, 7.0 Hz, 1H), 2.46 (dd, J=9.3, 5.3 Hz, 1H), 1.17 (t, J=7.1 Hz, 3H).

### trans-Ethyl 2-([2,2′-bipyrimidin]-5-yl)-3-(3,4-difluoro-5-methoxyphenyl)cyclopropane-1-carboxylate

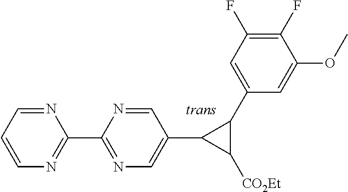 [SMILES: CCOC(=O)C1C(c2cnc(-c3ncccn3)nc2)C1c1cc(F)c(F)c(OC)c1]

A microwave vial equipped with a stir bar was charged with trans-ethyl 2-(2-chloropyrimidin-5-yl)-3-(3,4-difluoro-5-methoxy-phenyl)cyclopropanecarboxylate (92 mg, 0.25 mmol), [1,1′-bis(diphenylphosphino)ferrocene]dichloropalladium(II), 1:1 complex with dichloromethane (20 mg, 0.03 mmol), copper(I) iodide (5 mg , 0.030 mmol), 2-(tributylstannyl)pyrimidine (0.12 mL, 0.38 mmol) and dry 1,4-dioxane (1 mL). The reaction mixture was purged with nitrogen gas for 5 minutes. The vial was sealed and heated thermally at 110° C. for 16 hours behind a blast shield. The reaction mixture was cooled to rt, diluted with CH₂Cl₂ (10 mL), filtered through a plug of CELITE® and evaporated to a dark brown resin. The residue was partitioned between CH₃CN (20 mL) and hexanes (30 mL). The CH₃CN layer was evaporated to a resin. The resin was purified via normal phase SiO₂ chromatography (0-5% MeOH/CH₂Cl₂). The desired fractions were evaporated to give, as a mixture of diastereomers, racemic, trans-ethyl 2-([2,2′-bipyrimidin]-5-yl)-3-(3,4-difluoro-5-methoxyphenyl)cyclopropane-1-carboxylate as a tan solid (70 mg, 68% yield, m/z: 413 [M+H]⁺ observed). ¹H NMR (400 MHz, CDCl₃): δ 9.06-9.00 (m, 2H), 8.98-8.85 (m, 2H), 7.44 (t, J=4.9 Hz, 1H), 6.72-6.60 (m, 2H), 4.10-3.97 (m, 2H), 3.97-3.90 (m, 3H), 3.27 (t, J=6.0 Hz, 1H), 2.91-2.82 (m, 1H), 2.51 (dd, J=9.4, 5.4 Hz, 1H), 1.17-0.90 (m, 3H).

### Example 264: trans-4-(2-([2,2′-Bipyrimidin]-5-yl)cyclopropyl)-6,7-difluoro-1-(3-methoxypropyl)-1H-indazole

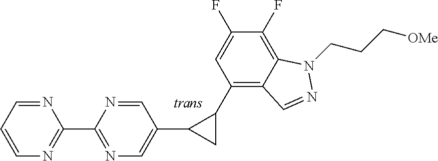 [SMILES: COCCCn1ncc2c(C3CC3c3cnc(-c4ncccn4)nc3)cc(F)c(F)c21]

### 4-Bromo-6,7-difluoro-1H-indazole

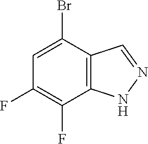 [SMILES: Fc1cc(Br)c2cn[nH]c2c1F]

A flask equipped with a magnetic stir bar and reflux condenser was charged with 6-bromo-2,3,4-trifluoro-benzaldehyde (1.00 g, 4.18 mmol) and 1,4-dioxane (10 mL), followed by hydrazine hydrate (0.61 mL, 12.6 mmol). The mixture was heated at 80° C. under a nitrogen atmosphere for 24 hours. The mixture was cooled to room temperature and evaporated under reduced pressure. The residue was dissolved in EtOAc (50 mL) and washed with water (3×15 mL). The combined organic phase was dried over Na₂SO₄, filtered and evaporated to give 4-bromo-6,7-difluoro-1H-indazole as a yellow solid, which was used in the next step without further purification (0.94 g, 96% yield, m/z: 233 [M+H]⁺ observed). ¹H NMR (400 MHz, CDCl₃) δ 10.76 (s, 1H), 8.10 (d, J=3.2 Hz, 1H), 7.23 (dd, J=9.9, 5.8 Hz, 1H).

### 4-Bromo-6,7-difluoro-1-(3-methoxypropylfindazole and 4-bromo-6,7-difluoro-2-(3-methoxypropyl)indazole

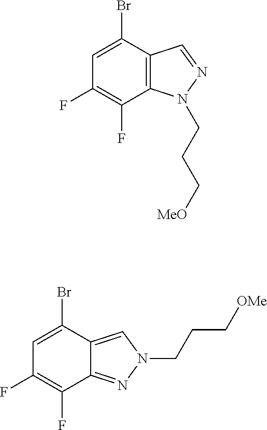 [SMILES: COCCCn1ncc2c(Br)cc(F)c(F)c21 ; COCCCn1cc2c(Br)cc(F)c(F)c2n1]

A suspension of 4-bromo-6,7-difluoro-1H-indazole (930 mg, 4.0 mmol), 1-bromo-3-methoxy-propane (0.67 mL, 6.0 mmol) and potassium carbonate (1.38 g, 10 mmol) in dry CH₃CN (20 mL) was heated at 40° C. for 24 hours. The reaction mixture was cooled to room temperature and the volatiles were evaporated. The residue was partitioned between water (50 mL) and EtOAc (50 mL). The combined organic phase was washed with water (2×50 mL) and saturated aqueous brine solution (10 mL). The combined organic phase was dried over Na₂SO₄, filtered and evaporated. The residue was purified via normal phase SiO₂ chromatography (20% EtOAc/Hexanes). The desired fractions were collected and evaporated to give 4-bromo-6,7-difluoro-1-(3-methoxypropyl)indazole as a yellow oil, which was used in the next step without further purification [faster eluting peak, 517 mg, 42% yield, m/z: 305 [M+H]⁺ observed, ¹H NMR (400 MHz, CDCl₃): δ 7.95 (d, J=2.1 Hz, 1H), 7.17 (dd, J=9.8, 5.7 Hz, 1H), 4.61 (t, J=6.8 Hz, 2H), 3.35 (t, J=6.0 Hz, 2H), 3.30 (s, 3H), 2.17 (p, J=6.4 Hz, 2H)] and 4-bromo-6,7-difluoro-2-(3-methoxypropyl)indazole as a red-orange oil [slower eluting peak, 471 mg, 38% yield, m/z: 305 [M+H]⁺ observed, ¹H NMR (400 MHz, CDCl₃): δ 7.96 (d, J=2.4 Hz, 1H), 7.14 (dd, J=10.1, 5.7 Hz, 1H), 4.53 (t, J=6.9 Hz, 2H), 3.35-3.30 (m, 5H), 2.31-2.23 (m, 2H)].

### 4-(2-(2-Chloropyrimidin-5-yl)cyclopropyl)-6,7-difluoro-1-(3-methoxypropyl)-1H-indazole

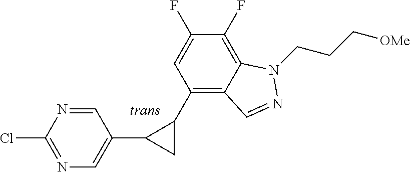 [SMILES: COCCCn1ncc2c(C3CC3c3cnc(Cl)nc3)cc(F)c(F)c21]

A microwave vial equipped with a stir bar was charged with 4-bromo-6,7-difluoro-1-(3-methoxypropyl)indazole (faster eluting fraction, 419 mg, 1.37 mmol), crude 2-chloro-5-[2-(4,4,5,5-tetramethyl-1,3,2-dioxaborolan-2-yl)cyclopropyl]pyrimidine (350 mg, 1.25 mmol), [1,1′-bis(diphenylphosphino)ferrocene]dichloropalladium(II), 1:1 complex with dichloromethane (102 mg, 0.12 mmol), cesium carbonate (1220.0 mg , 3.74 mmol) and THF-Water (9:1, 1.5 mL). The mixture was purged with nitrogen gas for 5 minutes. The vial was sealed and heated at 80° C. for 4 hours behind a blast shield. The reaction mixture was cooled to rt, diluted with CH₂Cl₂ (10 mL), filtered through a plug of CELITE®, rinsed and evaporated to give a dark brown viscous oil. The residue was purified via normal phase SiO₂ chromatography (0-10% MeOH/CH₂Cl₂). The desired fractions were collected and evaporated under reduced pressure to give trans-4-(2-(2-chloropyrimidin-5-yl)cyclopropyl)-6,7-difluoro-1-(3-methoxypropyl)-1H-indazole as a yellow viscous oil, which was used in the next step without further purification (310 mg, 65% yield, m/z: 379 [M+H]⁺ observed). ¹H NMR (400 MHz, CDCl₃): δ 8.48 (s, 2H), 7.96 (d, J=2.2 Hz, 1H), 6.66 (dd, J=11.2, 6.0 Hz, 1H), 4.62 (t, J=6.9 Hz, 2H), 3.37 (t, J=6.1 Hz, 2H), 3.31 (s, 3H), 2.53-2.47 (m, 1H), 2.26-2.13 (m, 3H), 1.71 (dt, J=8.9, 5.9 Hz, 1H), 1.62 (dt, J=8.9, 5.8 Hz, 1H).

### trans-4-(2-([2,2′-Bipyrimidin]-5-yl)cyclopropyl)-6,7-difluoro-1-(3-methoxypropyl)-1H-indazole

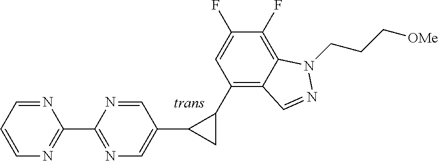 [SMILES: COCCCn1ncc2c(C3CC3c3cnc(-c4ncccn4)nc3)cc(F)c(F)c21]

A microwave vial equipped with a stir bar was charged with 4-[2-(2-chloropyrimidin-5-yl)cyclopropyl]-6,7-difluoro-1-(3-methoxypropyl)indazole (310 mg, 0.82 mmol), [1,1′-bis(diphenylphosphino)ferrocene]dichloropalladium(II), 1:1 complex with dichloromethane (67 mg , 0.08 mmol), copper(I) iodide (16 mg, 0.08 mmol), 2-(tributylstannyl)pyrimidine (0.39 mL, 1.23 mmol) and dry 1,4-dioxane (1 mL). The mixture was purged with nitrogen gas for 5 minutes. The vial was sealed and heated at 110° C. behind a blast shield for 16 hours. The mixture was cooled to rt, diluted with CH₂Cl₂ (10 mL), filtered through a plug of CELITE® and evaporated. The residue was partitioned between CH₃CN (20 mL) and hexanes (30 mL). The CH₃CN layer was washed with hexanes (2×30 mL) and evaporated under reduced pressure to give a dark brown resin. The resin was purified via normal phase SiO₂ chromatography (0-10% MeOH/CH₂Cl₂). The recovered material was further purified by reverse phase HPLC to give trans-6,7-difluoro-1-(3-methoxypropyl)-4-[2-(2-pyrimidin-2-ylpyrimidin-5-yl)cyclopropyl]indazole as a tan solid (167 mg, 48% yield, m/z: 423 [M+H]⁺ observed). ¹H NMR (400 MHz, CDCl₃): δ 9.03 (d, J=4.8 Hz, 2H), 8.84 (s, 2H), 7.97 (d, J=2.2 Hz, 1H), 7.44 (t, J=4.8 Hz, 1H), 6.71 (dd, J=11.2, 5.9 Hz, 1H), 4.62 (t, J=6.8 Hz, 2H), 3.37 (t, J=6.1 Hz, 2H), 3.31 (s, 3H), 2.64-2.57 (m, 1H), 2.38-2.31 (m, 1H), 2.18 (p, J=6.4 Hz, 2H), 1.82-1.70 (m, 2H).

### Example 265: trans-4-(2-([2,2′-Bipyrimidin]-5-yl)cyclopropyl)-6,7-difluoro-1-(3-methoxypropyl)-1H-indazole (single enantiomer I)

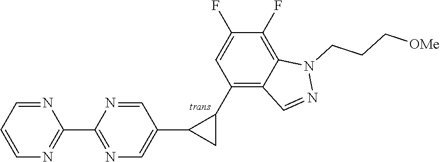 [SMILES: COCCCn1ncc2c(C3CC3c3cnc(-c4ncccn4)nc3)cc(F)c(F)c21]

### Example 266: trans-4-(2-([2,2′-Bipyrimidin]-5-yl)cyclopropyl)-6,7-difluoro-1-(3-methoxypropyl)-1H-indazole (single enantiomer II)

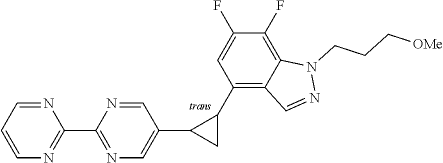 [SMILES: COCCCn1ncc2c(C3CC3c3cnc(-c4ncccn4)nc3)cc(F)c(F)c21]

A mixture of enantiomers of trans-4-(2-([2,2′-bipyrimidin]-5-yl)cyclopropyl)-6,7-difluoro-1-(3-methoxypropyl)-1H-indazole (160 mg) was separated by SFC (supercritical fluid chromatography) on a CHIRALCEL® OD-3 column using liquid CO₂ and 0.5% DEA in MeOH (60:40) to give trans-4-(2-([2,2′-bipyrimidin]-5-yl)cyclopropyl)-6,7-difluoro-1-(3-methoxypropyl)-1H-indazole (single enantiomer I) as a white solid (faster eluting enantiomer, 58 mg, 36%, m/z: 423 [M+H]⁺ observed), and trans-4-(2-([2,2′-bipyrimidin]-5-yl)cyclopropyl)-6,7-difluoro-1-(3-methoxypropyl)-1H-indazole (single enantiomer II) as a white solid (slower eluting enantiomer, 52 mg, 32%, m/z: 423 [M+H]⁺ observed).

### Example 265: trans-4-(2-([2,2′-Bipyrimidin]-5-yl)cyclopropyl)-6,7-difluoro-1-(3-methoxypropyl)-1H-indazole (single enantiomer I)

m/z: 423 [M+H]⁺ observed. ¹H NMR (400 MHz, CDCl₃): δ 9.03 (d, J=4.8 Hz, 2H), 8.84 (s, 2H), 7.97 (d, J=2.2 Hz, 1H), 7.44 (t, J=4.8 Hz, 1H), 6.71 (dd, J=11.2, 5.9 Hz, 1H), 4.62 (t, J=6.8 Hz, 2H), 3.37 (t, J=6.1 Hz, 2H), 3.31 (s, 3H), 2.64-2.57 (m, 1H), 2.38-2.31 (m, 1H), 2.18 (p, J=6.4 Hz, 2H), 1.82-1.70 (m, 2H).

### Example 266: trans-4-(2-([2,2′-Bipyrimidin]-5-yl)cyclopropyl)-6,7-difluoro-1-(3-methoxypropyl)-1H-indazole (single enantiomer II)

m/z: 423 [M+H]⁺ observed. ¹H NMR (400 MHz, CDCl₃): δ 9.03 (d, J=4.8 Hz, 2H), 8.84 (s, 2H), 7.97 (d, J=2.2 Hz, 1H), 7.44 (t, J=4.8 Hz, 1H), 6.71 (dd, J=11.2, 5.9 Hz, 1H), 4.62 (t, J=6.8 Hz, 2H), 3.37 (t, J=6.1 Hz, 2H), 3.31 (s, 3H), 2.64-2.57 (m, 1H), 2.38-2.31 (m, 1H), 2.18 (p, J=6.4 Hz, 2H), 1.82-1.70 (m, 2H).

The following examples were prepared in a similar manner as trans-4-(2-([2,2′-bipyrimidin]-5-yl)cyclopropyl)-6,7-difluoro-1-(3-methoxypropyl)-1H-indazole from 2-chloro-5-[2-(4,4,5,5-tetramethyl-1,3,2-dioxaborolan-2-yl)cyclopropyl]pyrimidine and an appropriate aryl bromide.

### Example 267: trans-6-(2-([2,2′-Bipyrimidin]-5-yl)cyclopropyl)-4-fluoro-2-(3-methoxypropyl)-2H-indazole

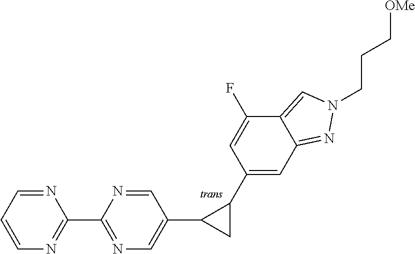 [SMILES: COCCCn1cc2c(F)cc(C3CC3c3cnc(-c4ncccn4)nc3)cc2n1]

m/z: 405 [M+H]⁺ observed. ¹H NMR (400 MHz, CDCl₃): δ 9.02 (d, J=4.9 Hz, 2H), 8.80 (s, 2H), 7.96 (s, 1H), 7.42 (t, J=4.8 Hz, 1H), 7.30 (s, 1H), 6.54 (dd, J=11.2, 0.9 Hz, 1H), 4.51 (t, J=6.8 Hz, 2H), 3.34-3.30 (m, 5H), 2.47-2.40 (m, 1H), 2.27 (h, J=6.7 Hz, 3H), 1.70 (ddt, J=25.0, 8.9, 5.9 Hz, 2H).

### Example 268: trans-6-(2-([2,2′-Bipyrimidin]-5-yl)cyclopropyl)-4-fluoro-2-(3-methoxypropyl)-2H-indazole (single enantiomer I)

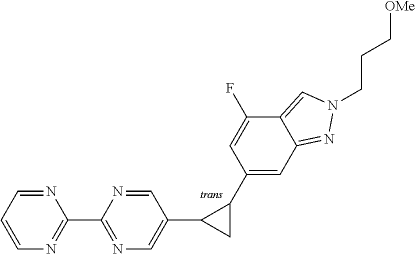 [SMILES: COCCCn1cc2c(F)cc(C3CC3c3cnc(-c4ncccn4)nc3)cc2n1]

### Example 269: trans-6-(2-([2,2′-Bipyrimidin]-5-yl)cyclopropyl)-4-fluoro-2-(3-methoxypropyl)-2H-indazole (single enantiomer II)

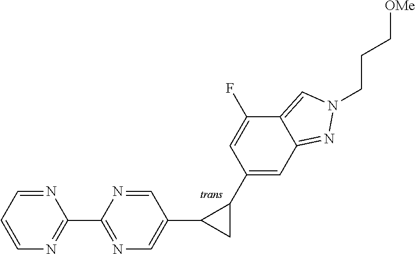 [SMILES: COCCCn1cc2c(F)cc(C3CC3c3cnc(-c4ncccn4)nc3)cc2n1]

A mixture of enantiomers of trans-6-(2-([2,2′-bipyrimidin]-5-yl)cyclopropyl)-4-fluoro-2-(3-methoxypropyl)-2H-indazole (84 mg) was separated by SFC (supercritical fluid chromatography) on a CHIRALCEL® OD-3 column using liquid CO₂ and 0.5% DEA in MeOH (60:40) to give trans-6-(2-([2,2′-bipyrimidin]-5-yl)cyclopropyl)-4-fluoro-2-(3-methoxypropyl)-2H-indazole (single enantiomer I) as a white solid (faster eluting enantiomer, 20 mg, 23%, m/z: 405 [M+H]⁺ observed), and trans-6-(2-([2,2′-bipyrimidin]-5-yl)cyclopropyl)-4-fluoro-2-(3-methoxypropyl)-2H-indazole (single enantiomer II) as a white solid (slower eluting enantiomer, 18 mg, 21%, m/z: 405 [M+H]⁺ observed).

### Example 268: trans-6-(2-([2,2′-Bipyrimidin]-5-yl)cyclopropyl)-4-fluoro-2-(3-methoxypropyl)-2H-indazole (single enantiomer I)

m/z: 405 [M+H]⁺ observed. ¹H NMR (400 MHz, CDCl₃) δ 9.02 (d, J=4.9 Hz, 2H), 8.80 (s, 2H), 7.96 (s, 1H), 7.42 (t, J=4.8 Hz, 1H), 7.30 (s, 1H), 6.54 (dd, J=11.2, 0.9 Hz, 1H), 4.51 (t, J=6.8 Hz, 2H), 3.34-3.30 (m, 5H), 2.47-2.40 (m, 1H), 2.27 (h, J=6.7 Hz, 3H), 1.70 (ddt, J=25.0, 8.9, 5.9 Hz, 2H).

### Example 269: trans-6-(2-([2,2′-Bipyrimidin]-5-yl)cyclopropyl)-4-fluoro-2-(3-methoxypropyl)-2H-indazole (single enantiomer II)

m/z: 405 [M+H]⁺ observed. ¹H NMR (400 MHz, CDCl₃) δ 9.02 (d, J=4.9 Hz, 2H), 8.80 (s, 2H), 7.96 (s, 1H), 7.42 (t, J=4.8 Hz, 1H), 7.30 (s, 1H), 6.54 (dd, J=11.2, 0.9 Hz, 1H), 4.51 (t, J=6.8 Hz, 2H), 3.34-3.30 (m, 5H), 2.47-2.40 (m, 1H), 2.27 (h, J=6.7 Hz, 3H), 1.70 (ddt, J=25.0, 8.9, 5.9 Hz, 2H).

### Example 270: trans-2-(2-([2,2′-Bipyrimidin]-5-yl)cyclopropyl)-4,6-difluorobenzo[d]thiazole

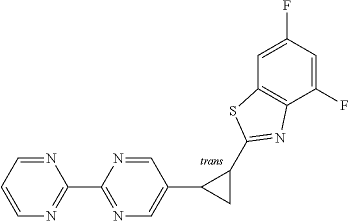 [SMILES: Fc1cc(F)c2nc(C3CC3c3cnc(-c4ncccn4)nc3)sc2c1]

m/z: 368 [M+H]⁺ observed. ¹H NMR (400 MHz, CDCl₃): δ 9.12-8.95 (m, 2H), 8.83 (s, 2H), 7.43 (td, J=4.8, 0.4 Hz, 1H), 7.38-7.28 (m, 1H), 7.08-6.89 (m, 1H), 3.00-2.86 (m, 1H), 2.77 (ddd, J=8.7, 5.7, 4.3 Hz, 1H), 2.16 (dt, J=9.2, 5.5 Hz, 1H), 1.87 (ddd, J=8.8, 6.4, 5.4 Hz, 1H).

### Example 271: trans-6-(2-([2,2′-Bipyrimidin]-5-yl)cyclopropyl)-4-fluoro-1-isopropyl-2-methyl-1H-benzo[d]imidazole

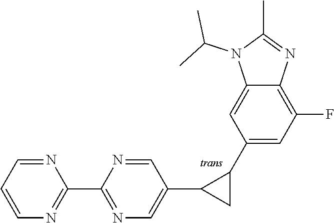 [SMILES: Cc1nc2c(F)cc(C3CC3c3cnc(-c4ncccn4)nc3)cc2n1C(C)C]

m/z: 389 [M+H]⁺ observed. ¹H NMR (400 MHz, CDCl₃): δ 9.02-8.95 (m, 2H), 8.78 (s, 2H), 7.48-7.35 (m, 1H), 7.10 (d, J=1.2 Hz, 1H), 6.72-6.57 (m, 1H), 4.63 (p, J=7.0 Hz, 1H), 2.61 (s, 3H), 2.51-2.37 (m, 1H), 2.27-2.17 (m, 1H), 1.67 (dt, J=8.8, 5.6 Hz, 2H), 1.62 (dd, J=7.0, 3.1 Hz, 6H).

### Example 272: trans-6-(2-([2,2′-Bipyrimidin]-5-yl)cyclopropyl)-4-fluoro-2-methylbenzo[d]thiazole

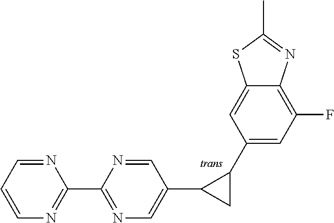 [SMILES: Cc1nc2c(F)cc(C3CC3c3cnc(-c4ncccn4)nc3)cc2s1]

m/z: 364 [M+H]⁺ observed. ¹H NMR (400 MHz, CDCl₃): δ 9.08-8.93 (m, 2H), 8.79 (d, J=0.4 Hz, 2H), 7.49-7.34 (m, 2H), 6.95 (ddd, J=11.2, 1.6, 0.4 Hz, 1H), 2.85 (s, 3H), 2.46 (td, J=7.8, 4.5 Hz, 1H), 2.33-2.25 (m, 1H), 1.78-1.65 (m, 2H).

### Example 273: trans-5-(2-([2,2′-Bipyrimidin]-5-yl)cyclopropyl)-7-fluoro-1-(3-methoxypropyl)-1H-benzo[d]imidazole

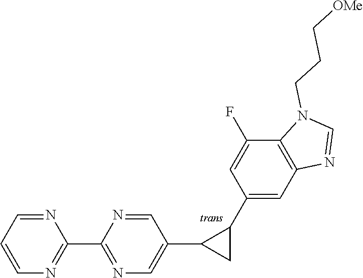 [SMILES: COCCCn1cnc2cc(C3CC3c3cnc(-c4ncccn4)nc3)cc(F)c21]

m/z: 405 [M+H]⁺ observed. ¹H NMR (400 MHz, CDCl₃) δ 9.02 (d, J=4.8 Hz, 2H), 8.80 (s, 2H), 7.85 (s, 1H), 7.43 (t, J=4.9 Hz, 1H), 7.08-7.06 (m, 1H), 6.76 (dd, J=11.4, 1.4 Hz, 1H), 4.30 (t, J=6.7 Hz, 2H), 3.34 (s, 3H), 3.29 (t, J=5.6 Hz, 2H), 2.49 (dt, J=8.8, 5.9 Hz, 1H), 2.31-2.25 (m, 1H), 2.10 (p, J=6.3 Hz, 2H), 1.76-1.65 (m, 2H).

### Example 274: trans-6-(2-([2,2′-Bipyrimidin]-5-yl)cyclopropyl)-4-fluoro-1-(3-methoxypropyl)-1H-benzo[d]imidazole

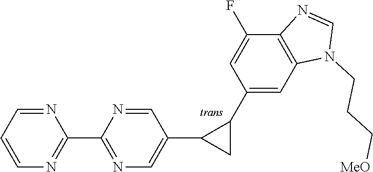 [SMILES: COCCCn1cnc2c(F)cc(C3CC3c3cnc(-c4ncccn4)nc3)cc21]

m/z: 405 [M+H]⁺ observed. ¹H NMR (400 MHz, CDCl₃): δ 9.02 (d, J=4.8 Hz, 2H), 8.79 (s, 2H), 7.82 (s, 1H), 7.42 (t, J=4.8 Hz, 1H), 7.39 (s, 1H), 6.85 (d, J=12.2 Hz, 1H), 4.41 (t, J=6.7 Hz, 2H), 3.34 (s, 3H), 3.29 (t, J=5.7 Hz, 2H), 2.50-2.43 (m, 1H), 2.29-2.22 (m, 1H), 2.12 (p, J=6.2 Hz, 2H), 1.69 (ddt, J=18.4, 8.8, 5.8 Hz, 2H).

### Example 275: trans-6-(2-([2,2′-Bipyrimidin]-5-yl)cyclopropyl)-4-fluoro-1-(3-methoxypropyl)-1H-indazole

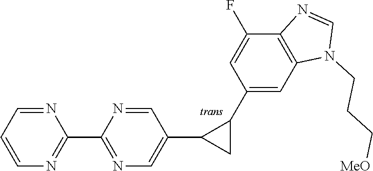 [SMILES: COCCCn1cnc2c(F)cc(C3CC3c3cnc(-c4ncccn4)nc3)cc21]

m/z: 405 [M+H]⁺ observed. ¹H NMR (400 MHz, CDCl₃): δ 9.02 (d, J=4.8 Hz, 2H), 8.80 (s, 2H), 8.02 (d, J=0.9 Hz, 1H), 7.43 (t, J=4.8 Hz, 1H), 7.07 (s, 1H), 6.57 (d, J=10.8 Hz, 1H), 4.46 (t, J=6.6 Hz, 2H), 3.31-3.26 (m, 5H), 2.52-2.45 (m, 1H), 2.35-2.28 (m, 1H), 2.17 (p, J=6.3 Hz, 2H), 1.73 (ddt, J=14.7, 8.9, 6.0 Hz, 2H).

### Example 276: trans-4-(2-([2,2′-Bipyrimidin]-5-yl)cyclopropyl)-7-fluoro-1-(3-methoxypropyl)-1H-indazole

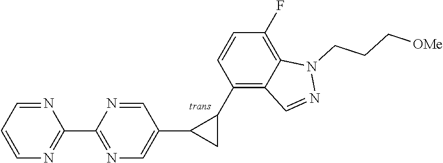 [SMILES: COCCCn1ncc2c(C3CC3c3cnc(-c4ncccn4)nc3)ccc(F)c21]

m/z: 405 [M+H]⁺ observed. ¹H NMR (400 MHz, CDCl₃): δ 9.03 (d, J=4.9 Hz, 2H), 8.85 (s, 2H), 8.01 (d, J=2.3 Hz, 1H), 7.43 (t, J=4.8 Hz, 1H), 6.98 (dd, J=11.7, 7.9 Hz, 1H), 6.77 (dd, J=7.8, 3.6 Hz, 1H), 4.65 (t, J=6.9 Hz, 2H), 3.37 (t, J=6.1 Hz, 2H), 3.31 (s, 3H), 2.67-2.59 (m, 1H), 2.39-2.29 (m, 1H), 2.18 (p, J=6.5 Hz, 2H), 1.85-1.77 (m, 1H), 1.75-1.68 (m, 1H).

### Example 277: trans-4-(2-([2,2′-Bipyrimidin]-5-yl)cyclopropyl)-7-fluoro-2-(3-methoxypropyl)-2H-indazole

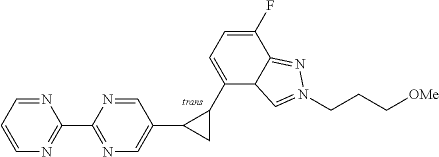

m/z: 405 [M+H]⁺ observed. ¹H NMR (400 MHz, CDCl₃): δ 9.03 (d, J=4.8 Hz, 2H), 8.84 (s, 2H), 7.95 (d, J=2.7 Hz, 1H), 7.44 (t, J=4.8 Hz, 1H), 6.88 (dd, J=11.1, 7.6 Hz, 1H), 6.74 (ddd, J=7.6, 3.9, 0.8 Hz, 1H), 4.54 (t, J=6.9 Hz, 2H), 3.35-3.30 (m, 5H), 2.56-2.50 (m, 1H), 2.32-2.22 (m, 3H), 1.85-1.78 (m, 1H), 1.70-1.63 (m, 1H).

### Example 278: trans-5-(2-([2,2′-Bipyrimidin]-5-yl)cyclopropyl)-2-methylbenzo[d]thiazole

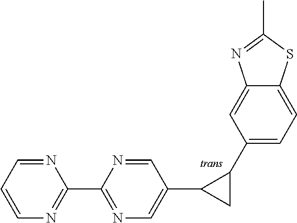 [SMILES: Cc1nc2cc(C3CC3c3cnc(-c4ncccn4)nc3)ccc2s1]

m/z: 346 [M+H]⁺ observed. ¹H NMR (400 MHz, CDCl₃): δ 9.01 (d, J=4.9 Hz, 2H), 8.79 (d, J=0.4 Hz, 2H), 7.80-7.70 (m, 2H), 7.42 (t, J=4.9 Hz, 1H), 7.20 (dd, J=8.2, 1.9 Hz, 1H), 2.83 (s, 3H), 2.49 (ddd, J=8.9, 6.2, 4.5 Hz, 1H), 2.28 (ddd, J=8.7, 6.0, 4.5 Hz, 1H), 1.79-1.66 (m, 2H).

### Example 279: Biological Examples

### HBsAg Assay

Inhibition of HBsAg was determined in HepG2.2.15 cells. Cells were maintained in culture medium containing 10% fetal calf serum, G414, Glutamine, penicillin/streptomycin. Cells were seeded in 96-well collagen-coated plate at a density of 30,000 cells/well. Serially diluted compounds were added to cells next day at the final DMSO concentration of 0.5%. Cells were incubated with compounds for 2-3 days, after which medium was removed. Fresh medium containing compounds was added to cells for additional 3-4 days. At day 6 after exposure of compounds, supernatant was collected, the HBsAg immunoassay (microplate-based chemiluminescence immunoassay kits, CLIA, Autobio Diagnosics Co., Zhengzhou, China, Catalog # CL0310-2) was used to determine the level of HBsAg according to manufactory instruction. Dose-response curves were generated and the EC₅₀ value (effective concentrations that achieved 50% inhibitory effect) were determined using XLfit software. In addition, cells were seeded at a density of 5,000 cells/well for determination of cell viability in the presence and absence of compounds by using CellTiter-Glo reagent (Promega).

Table 1 illustrates EC₅₀ values obtained by the HBsAg assay for selected compounds.

TABLE 1

| | | | sAg |
|---|---|---|---|
| Ex. | | | EC₅₀, |
| No. | Structure | Nomenclature | μM |
| 1 | 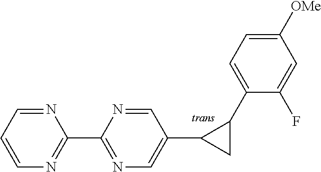 [SMILES: COc1ccc(C2CC2c2cnc(-c3ncccn3)nc2)c(F)c1] | trans-5-(2-(2-fluoro-4- methoxyphenyl)cyclopropyl)- 2,2-bipyrimidine | 0.2 |
| 2 | 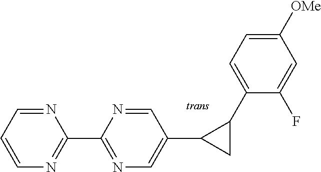 [SMILES: COc1ccc(C2CC2c2cnc(-c3ncccn3)nc2)c(F)c1] | trans-5-(2-(2-fluoro-4- methoxyphenyl)cyclopropyl)- 2,2'-bipyrimidine (single enantiomer I) | 0.23 |
| 3 | 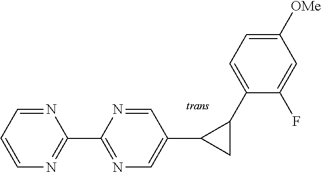 [SMILES: COc1ccc(C2CC2c2cnc(-c3ncccn3)nc2)c(F)c1] | trans-5-(2-(2-fluoro-4- methoxyphenyl)cyclopropyl)- 2,2'-bipyrimidine (single enantiomer II) | 0.19 |
| 4 | 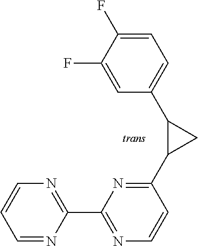 [SMILES: Fc1ccc(C2CC2c2ccnc(-c3ncccn3)n2)cc1F] | trans-4-(2-(3,4- difluorophenyl)cyclopropyl)- 2,2'-bipyrimidine | 7 |
| 5 | 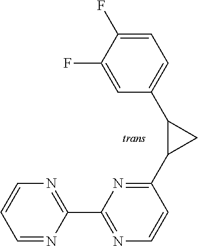 [SMILES: Fc1ccc(C2CC2c2ccnc(-c3ncccn3)n2)cc1F] | trans-4-(2-(3,4- difluorophenyl)cyclopropyl)- 2,2'-bipyrimidine (single enantiomer I) | 5 |
| 6 | 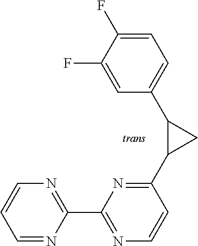 [SMILES: Fc1ccc(C2CC2c2ccnc(-c3ncccn3)n2)cc1F] | trans-4-(2-(3,4- difluorophenyl)cyclopropyl)- 2,2'-bipyrimidine (single enantiomer II) | 10 |
| 7 | 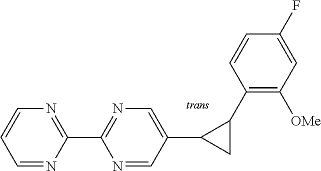 [SMILES: COc1cc(F)ccc1C1CC1c1cnc(-c2ncccn2)nc1] | trans-5-(2-(4-fluoro-2- methoxyphenyl)cyclopropyl)- 2,2'-bipyrimidine | 0.37 |
| 8 | 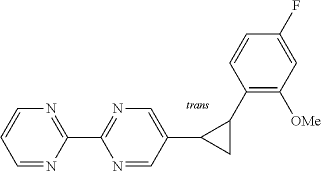 [SMILES: COc1cc(F)ccc1C1CC1c1cnc(-c2ncccn2)nc1] | trans-5-(2-(4-fluoro-2- methoxyphenyl)cyclopropyl)- 2,2'-bipyrimidine (single enantiomer I) | 0.45 |
| 9 | 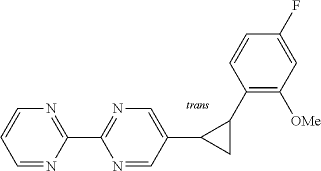 [SMILES: COc1cc(F)ccc1C1CC1c1cnc(-c2ncccn2)nc1] | trans-5-(2-(4-fluoro-2- methoxyphenyl)cyclopropyl)- 2,2'-bipyrimidine (single enantiomer II) | 0.085 |
| 10 | 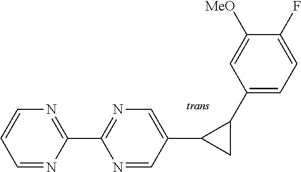 [SMILES: COc1cc(C2CC2c2cnc(-c3ncccn3)nc2)ccc1F] | trans-5-(2-(4-fluoro-3- methoxyphenyl)cyclopropyl)- 2,2'-bipyrimidine (single enantiomer I) | 0.04 |
| 11 | 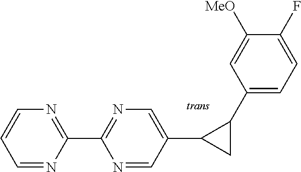 [SMILES: COc1cc(C2CC2c2cnc(-c3ncccn3)nc2)ccc1F] | trans-5-(2-(4-fluoro-3- methoxyphenyl)cyclopropyl)- 2,2'-bipyrimidine (single enantiomer II) | 0.73 |
| 12 | 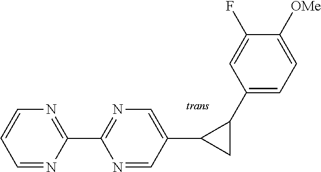 [SMILES: COc1ccc(C2CC2c2cnc(-c3ncccn3)nc2)cc1F] | trans-5-(2-(3-fluoro-4- methoxyphenyl)cyclopropyl)- 2,2'-bipyrimidine (single enantiomer I) | 0.11 |
| 13 | 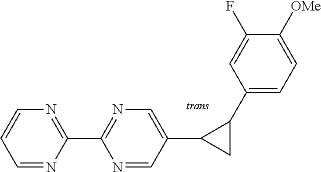 [SMILES: COc1ccc(C2CC2c2cnc(-c3ncccn3)nc2)cc1F] | trans-5-(2-(3-fluoro-4- methoxyphenyl)cyclopropyl)- 2,2'-bipyrimidine (single enantiomer II) | 0.082 |
| 14 | 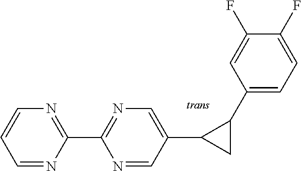 [SMILES: Fc1ccc(C2CC2c2cnc(-c3ncccn3)nc2)cc1F] | trans-5-(2-(3,4- difluorophenyl)cyclopropyl)- 2,2'-bipyrimidine (single enantiomer I) | 1 |
| 15 | 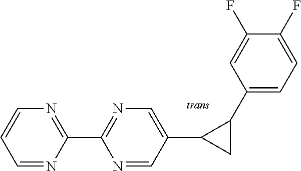 [SMILES: Fc1ccc(C2CC2c2cnc(-c3ncccn3)nc2)cc1F] | trans-5-(2-(3,4- difluorophenyl)cyclopropyl)- 2,2'-bipyrimidine (single enantiomer II) | 0.076 |
| 16 | 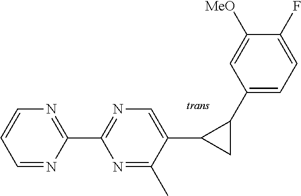 [SMILES: COc1cc(C2CC2c2cnc(-c3ncccn3)nc2C)ccc1F] | trans-5-(2-(4-fluoro-3- methoxyphenyl)cyclopropyl)- 4-methyl-2,2'-bipyrimidine | 0.75 |
| 17 | 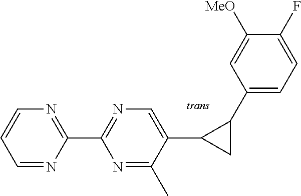 [SMILES: COc1cc(C2CC2c2cnc(-c3ncccn3)nc2C)ccc1F] | trans-5-(2-(4-fluoro-3- methoxyphenyl)cyclopropyl)- 4-methyl-2,2'-bipyrimidine (single enantiomer I) | 0.65 |
| 18 | 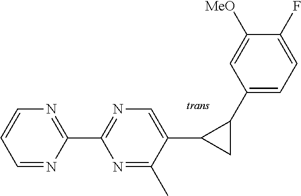 [SMILES: COc1cc(C2CC2c2cnc(-c3ncccn3)nc2C)ccc1F] | trans-5-(2-(4-fluoro-3- methoxyphenyl)cyclopropyl)- 4-methyl-2,2'-bipyrimidine (single enantiomer II) | 0.89 |
| 19 | 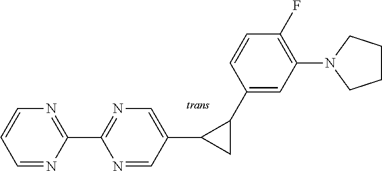 [SMILES: Fc1ccc(C2CC2c2cnc(-c3ncccn3)nc2)cc1N1CCCC1] | trans-5-(2-(4-fluoro-3- (pyrrolidin-1- yl)phenyl)cyclopropyl)-2,2'- bipyrimidine (single enantiomer I) | 0.039 |
| 20 | 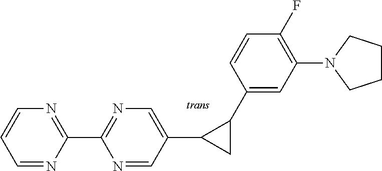 [SMILES: Fc1ccc(C2CC2c2cnc(-c3ncccn3)nc2)cc1N1CCCC1] | trans-5-(2-(4-fluoro-3- (pyrrolidin-1- yl)phenyl)cyclopropyl)-2,2'- bipyrimidine (single enantiomer II) | 0.023 |
| 21 | 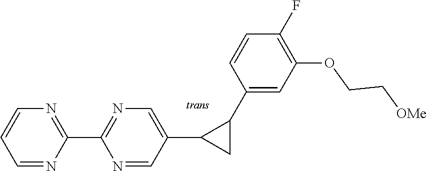 [SMILES: COCCOc1cc(C2CC2c2cnc(-c3ncccn3)nc2)ccc1F] | trans-5-(2-(4-fluoro-3-(2- methoxyethoxy)phenyl) cyclopropyl)-2,2'-bipyrimidine (single enantiomer I) | 0.49 |
| 22 | 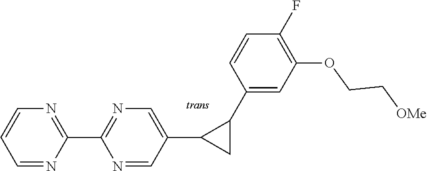 [SMILES: COCCOc1cc(C2CC2c2cnc(-c3ncccn3)nc2)ccc1F] | trans-5-(2-(4-fluoro-3-(2- methoxyethoxy)phenyl) cyclopropyl)-2,2'-bipyrimidine (single enantiomer II) | 0.059 |
| 23 | 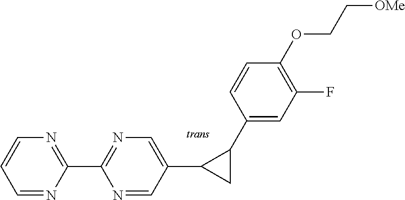 [SMILES: COCCOc1ccc(C2CC2c2cnc(-c3ncccn3)nc2)cc1F] | trans-5-(2-(3-fluoro-4-(2- methoxyethoxy)phenyl) cyclopropyl)-2,2'-bipyrimidine (single enantiomer I) | 0.095 |
| 24 | 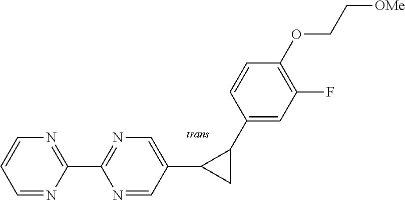 [SMILES: COCCOc1ccc(C2CC2c2cnc(-c3ncccn3)nc2)cc1F] | trans-5-(2-(3-fluoro-4-(2- methoxyethoxy)phenyl) cyclopropyl)-2,2'-bipyrimidine (single enantiomer II) | 0.15 |
| 25 | 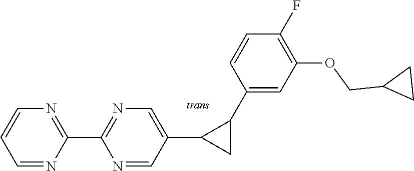 [SMILES: Fc1ccc(C2CC2c2cnc(-c3ncccn3)nc2)cc1OCC1CC1] | trans-5-(2-(3- (cyclopropylmethoxy)-4- fluorophenyl)cyclopropyl)- 2,2'-bipyrimidine (single enantiomer I) | 0.27 |
| 26 | 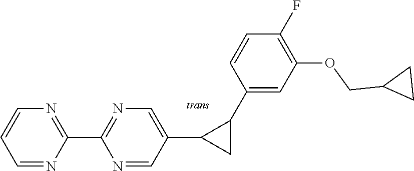 [SMILES: Fc1ccc(C2CC2c2cnc(-c3ncccn3)nc2)cc1OCC1CC1] | trans-5-(2-(3- (cyclopropylmethoxy)-4- fluorophenyl)cyclopropyl)- 2,2'-bipyrimidine (single enantiomer II) | 0.029 |
| 27 | 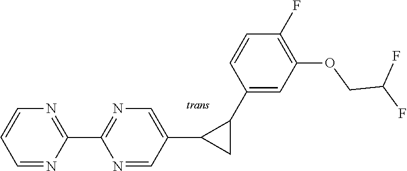 [SMILES: Fc1ccc(C2CC2c2cnc(-c3ncccn3)nc2)cc1OCC(F)F] | trans-5-(2-(3-(2,2- difluoroethoxy)-4- fluorophenyl)cyclopropyl)- 2,2'-bipyrimidine (single enantiomer I) | 0.63 |
| 28 | 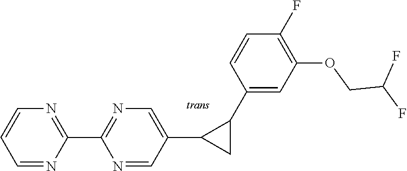 [SMILES: Fc1ccc(C2CC2c2cnc(-c3ncccn3)nc2)cc1OCC(F)F] | trans-5-(2-(3-(2,2- difluoroethoxy)-4- fluorophenyl)cyclopropyl)- 2,2'-bipyrimidine (single enantiomer II) | 0.056 |
| 29 | 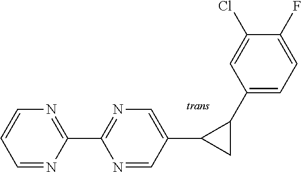 [SMILES: Fc1ccc(C2CC2c2cnc(-c3ncccn3)nc2)cc1Cl] | trans-5-(2-(3-chloro-4- fluorophenyl)cyclopropyl)- 2,2'-bipyrimidine (single enantiomer I) | 0.78 |
| 30 | 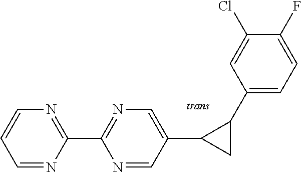 [SMILES: Fc1ccc(C2CC2c2cnc(-c3ncccn3)nc2)cc1Cl] | trans-5-(2-(3-chloro-4- fluorophenyl)cyclopropyl)- 2,2'-bipyrimidine (single enantiomer II) | 0.08 |
| 31 | 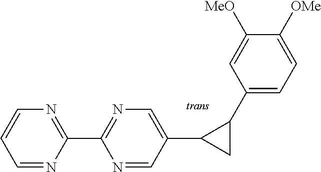 [SMILES: COc1ccc(C2CC2c2cnc(-c3ncccn3)nc2)cc1OC] | trans-5-(2-(3,4- dimethoxyphenyl)cyclopropyl)- 2,2'-bipyrimidine (single enantiomer I) | 0.33 |
| 32 | 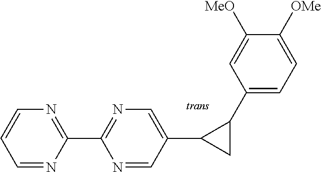 [SMILES: COc1ccc(C2CC2c2cnc(-c3ncccn3)nc2)cc1OC] | trans-5-(2-(3,4- dimethoxyphenyl)cyclopropyl)- 2,2'-bipyrimidine (single enantiomer II) | 0.48 |
| 33 | 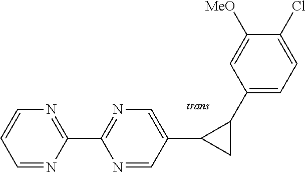 [SMILES: COc1cc(C2CC2c2cnc(-c3ncccn3)nc2)ccc1Cl] | trans-5-(2-(4-chloro-3- methoxyphenyl)cyclopropyl)- 2,2'-bipyrimidine (single enantiomer I) | 0.031 |
| 34 | 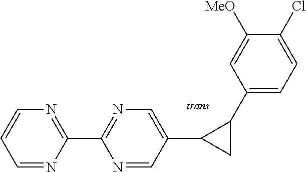 [SMILES: COc1cc(C2CC2c2cnc(-c3ncccn3)nc2)ccc1Cl] | trans-5-(2-(4-chloro-3- methoxyphenyl)cyclopropyl)- 2,2'-bipyrimidine (single enantiomer II) | 0.15 |
| 35 | 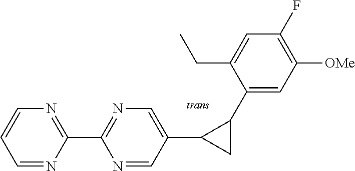 [SMILES: CCc1cc(F)c(OC)cc1C1CC1c1cnc(-c2ncccn2)nc1] | trans-5-(2-(2-ethyl-4-fluoro-5- methoxyphenyl)cyclopropyl)- 2,2'-bipyrimidine (single enantiomer I) | 0.55 |
| 36 | 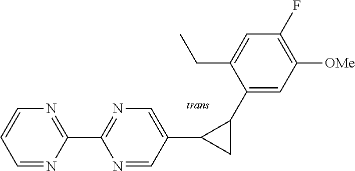 [SMILES: CCc1cc(F)c(OC)cc1C1CC1c1cnc(-c2ncccn2)nc1] | trans-5-(2-(2-ethyl-4-fluoro-5- methoxyphenyl)cyclopropyl)- 2,2'-bipyrimidine (single enantiomer II) | 0.72 |
| 37 | 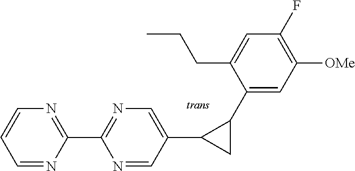 [SMILES: CCCc1cc(F)c(OC)cc1C1CC1c1cnc(-c2ncccn2)nc1] | trans-5-(2-(4-fluoro-5- methoxy-2- propylphenyl)cyclopropyl)- 2,2'-bipyrimidine (single enantiomer I) | 2 |
| 38 | 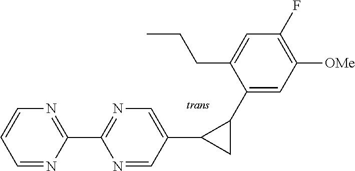 [SMILES: CCCc1cc(F)c(OC)cc1C1CC1c1cnc(-c2ncccn2)nc1] | trans-5-(2-(4-fluoro-5- methoxy-2- propylphenyl)cyclopropyl)- 2,2'-bipyrimidine (single enantiomer II) | 0.15 |
| 39 | 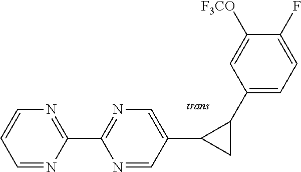 [SMILES: Fc1ccc(C2CC2c2cnc(-c3ncccn3)nc2)cc1OC(F)(F)F] | trans-5-(2-(4-fluoro-3- (trifluoromethoxy)phenyl) cyclopropyl)-2,2'-bipyrimidine (single enantiomer I) | 2 |
| 40 | 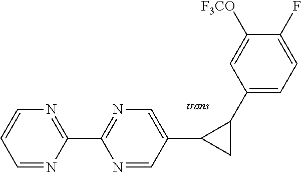 [SMILES: Fc1ccc(C2CC2c2cnc(-c3ncccn3)nc2)cc1OC(F)(F)F] | trans-5-(2-(4-fluoro-3- (trifluoromethoxy)phenyl) cyclopropyl)-2,2'-bipyrimidine (single enantiomer II) | 0.11 |
| 41 | 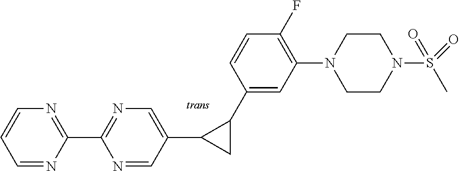 [SMILES: CS(=O)(=O)N1CCN(c2cc(C3CC3c3cnc(-c4ncccn4)nc3)ccc2F)CC1] | trans-5-(2-(4-fluoro-3-(4- (methylsulfonyl)piperazin-1- yl)phenyl)cyclopropyl)-2,2'- bipyrimidine (single enantiomer I) | 4 |
| 42 | 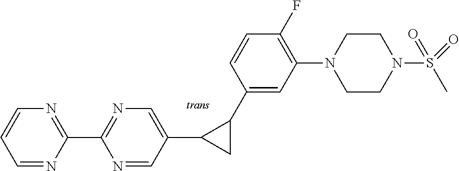 [SMILES: CS(=O)(=O)N1CCN(c2cc(C3CC3c3cnc(-c4ncccn4)nc3)ccc2F)CC1] | trans-5-(2-(4-fluoro-3-(4- (methylsulfonyl)piperazin-1- yl)phenyl)cyclopropyl)-2,2'- bipyrimidine (single enantiomer II) | 0.22 |
| 43 | 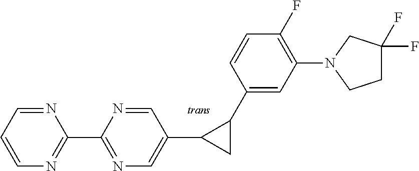 [SMILES: Fc1ccc(C2CC2c2cnc(-c3ncccn3)nc2)cc1N1CCC(F)(F)C1] | trans-5-(2-(3-(3,3- difluoropyrrolidin-1-yl)-4- fluorophenyl)cyclopropyl)- 2,2'-bipyrimidine (single enantiomer I) | 0.25 |
| 44 | 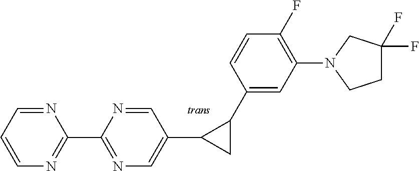 [SMILES: Fc1ccc(C2CC2c2cnc(-c3ncccn3)nc2)cc1N1CCC(F)(F)C1] | trans-5-(2-(3-(3,3- difluoropyrrolidin-1-yl)-4- fluorophenyl)cyclopropyl)- 2,2'-bipyrimidine (single enantiomer II) | 0.11 |
| 45 | 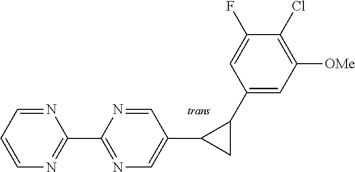 [SMILES: COc1cc(C2CC2c2cnc(-c3ncccn3)nc2)cc(F)c1Cl] | trans-5-(2-(4-chloro-3- fluoro-5- methoxyphenyl)cyclopropyl)- 2,2'-bipyrimidine (single enantiomer I) | 0.27 |
| 46 | 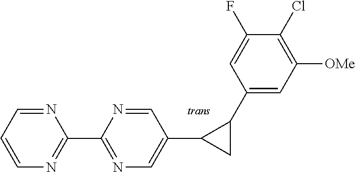 [SMILES: COc1cc(C2CC2c2cnc(-c3ncccn3)nc2)cc(F)c1Cl] | trans-5-(2-(4-chloro-3- fluoro-5- methoxyphenyl)cyclopropyl)- 2,2'-bipyrimidine (single enantiomer II) | 0.035 |
| 47 | 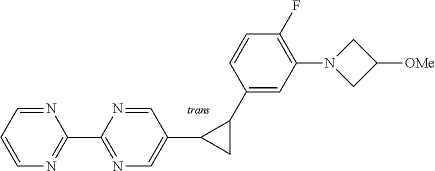 [SMILES: COC1CN(c2cc(C3CC3c3cnc(-c4ncccn4)nc3)ccc2F)C1] | trans-5-(2-(4-fluoro-3-(3- methoxyazetidin-1- yl)phenyl)cyclopropyl)-2,2'- bipyrimidine (single enantiomer I) | 0.37 |
| 48 | 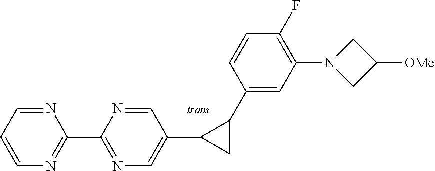 [SMILES: COC1CN(c2cc(C3CC3c3cnc(-c4ncccn4)nc3)ccc2F)C1] | trans-5-(2-(4-fluoro-3-(3- methoxyazetidin-1- yl)phenyl)cyclopropyl)-2,2'- bipyrimidine (single enantiomer II) | 0.032 |
| 49 | 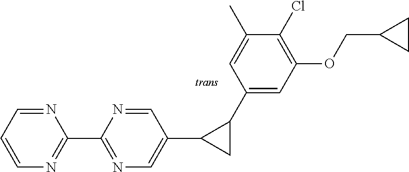 [SMILES: Cc1cc(C2CC2c2cnc(-c3ncccn3)nc2)cc(OCC2CC2)c1Cl] | trans-5-((2-(4-chloro-3- (cyclopropylmethoxy)-5- methylphenyl)cyclopropyl)- 2,2'-bipyrimidine (single enantiomer I) | 0.17 |
| 50 | 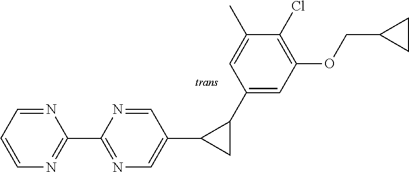 [SMILES: Cc1cc(C2CC2c2cnc(-c3ncccn3)nc2)cc(OCC2CC2)c1Cl] | trans-5-((2-(4-chloro-3- (cyclopropylmethoxy)-5- methylphenyl)cyclopropyl)- 2,2'-bipyrimidine (single enantiomer II) | 2 |
| 51 | 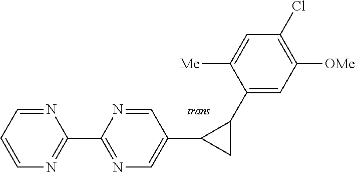 [SMILES: COc1cc(C2CC2c2cnc(-c3ncccn3)nc2)c(C)cc1Cl] | trans-5-(2-(4-chloro-5- methoxy-2- methylphenyl)cyclopropyl)- 2,2'-bipyrimidine (single enantiomer I) | 0.29 |
| 52 | 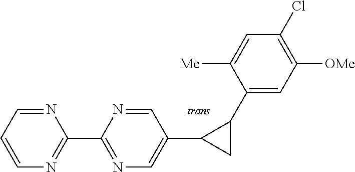 [SMILES: COc1cc(C2CC2c2cnc(-c3ncccn3)nc2)c(C)cc1Cl] | trans-5-(2-(4-chloro-5- methoxy-2- methylphenyl)cyclopropyl)- 2,2'-bipyrimidine (single enantiomer II) | 1 |
| 53 | 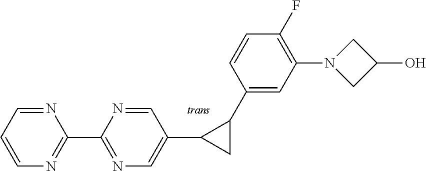 [SMILES: OC1CN(c2cc(C3CC3c3cnc(-c4ncccn4)nc3)ccc2F)C1] | trans-1-(5-(2-([2,2- bipyrimidin]-5- yl)cyclopropyl)-2- fluorophenyl)azetidin-3-ol (single enantiomer I) | 0.5 |
| 54 | 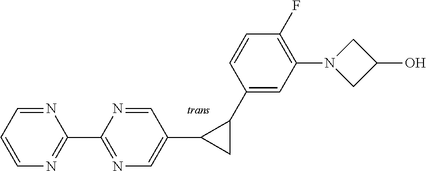 [SMILES: OC1CN(c2cc(C3CC3c3cnc(-c4ncccn4)nc3)ccc2F)C1] | trans-1-(5-(2-([2,2- bipyrimidin]-5- yl)cyclopropyl)-2- fluorophenyl)azetidin-3-ol (single enantiomer II) | 0.22 |
| 55 | 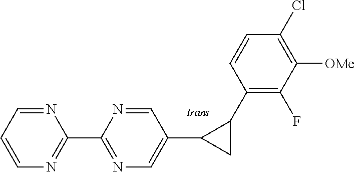 [SMILES: COc1c(Cl)ccc(C2CC2c2cnc(-c3ncccn3)nc2)c1F] | trans-5-(2-(4-chloro-2- fluoro-3- methoxyphenyl)cyclopropyl)- 2,2'-bipyrimidine (single enantiomer I) | 1 |
| 56 | 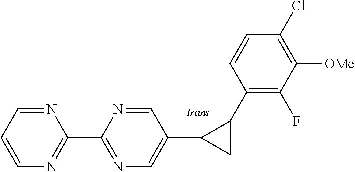 [SMILES: COc1c(Cl)ccc(C2CC2c2cnc(-c3ncccn3)nc2)c1F] | trans-5-(2-(4-chloro-2- fluoro-3- methoxyphenyl)cyclopropyl)- 2,2'-bipyrimidine (single enantiomer II) | 0.47 |
| 57 | 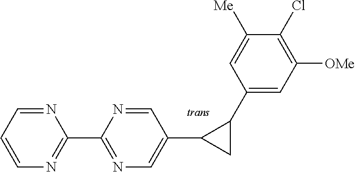 [SMILES: COc1cc(C2CC2c2cnc(-c3ncccn3)nc2)cc(C)c1Cl] | trans-5-(2-(4-chloro-3- methoxy-5- methylphenyl)cyclopropyl)- 2,2'-bipyrimidine (single enantiomer I) | 0.063 |
| 58 | 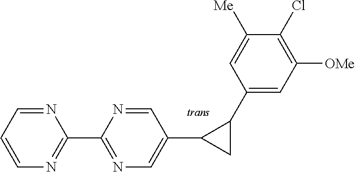 [SMILES: COc1cc(C2CC2c2cnc(-c3ncccn3)nc2)cc(C)c1Cl] | trans-5-(2-(4-chloro-3- methoxy-5- methylphenyl)cyclopropyl)- 2,2'-bipyrimidine (single enantiomer II) | 0.29 |
| 59 | 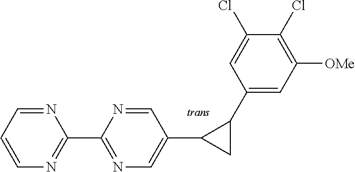 [SMILES: COc1cc(C2CC2c2cnc(-c3ncccn3)nc2)cc(Cl)c1Cl] | trans-5-(2-(3,4-dichloro-5- methoxyphenyl)cyclopropyl)- 2,2'-bipyrimidine (single enantiomer I) | 0.032 |
| 60 | 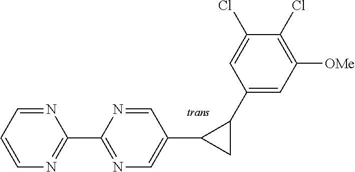 [SMILES: COc1cc(C2CC2c2cnc(-c3ncccn3)nc2)cc(Cl)c1Cl] | trans-5-(2-(3,4-dichloro-5- methoxyphenyl)cyclopropyl)- 2,2'-bipyrimidine (single enantiomer II) | 0.25 |
| 61 | 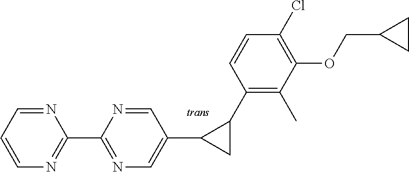 [SMILES: Cc1c(C2CC2c2cnc(-c3ncccn3)nc2)ccc(Cl)c1OCC1CC1] | trans-5-(2-(4-chloro-3- (cyclopropylmethoxy)-2- methylphenyl)cyclopropyl)- 2,2'-bipyrimidine (single enantiomer I) | 0.43 |
| 62 | 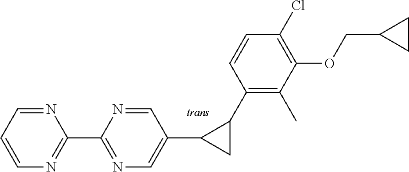 [SMILES: Cc1c(C2CC2c2cnc(-c3ncccn3)nc2)ccc(Cl)c1OCC1CC1] | trans-5-(2-(4-chloro-3- (cyclopropylmethoxy)-2- methylphenyl)cyclopropyl)- 2,2'-bipyrimidine (single enantiomer II) | 49 |
| 63 | 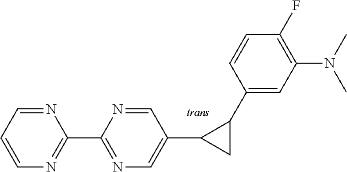 [SMILES: CN(C)c1cc(C2CC2c2cnc(-c3ncccn3)nc2)ccc1F] | trans-5-(2-([2,2'- bipyrimidin]-5- yl)cyclopropyl)-2-fluoro- N,N-dimethylaniline (single enantiomer I) | 0.39 |
| 64 | 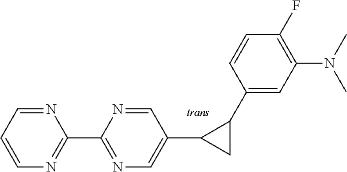 [SMILES: CN(C)c1cc(C2CC2c2cnc(-c3ncccn3)nc2)ccc1F] | trans-5-(2-([2,2'- bipyrimidin]-5- yl)cyclopropyl)-2-fluoro- N,N-dimethylaniline (single enantiomer II) | 3 |
| 65 | 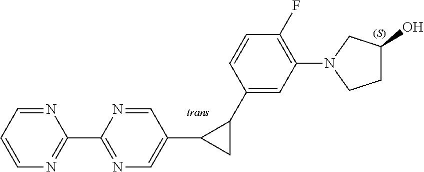 | trans-(3S)-1-(5-(2-([2,2'- bipyrimidin]-5- yl)cyclopropyl)-2- fluorophenyl)pyrrolidin-3-ol (single diastereomer I) | 0.58 |
| 66 | 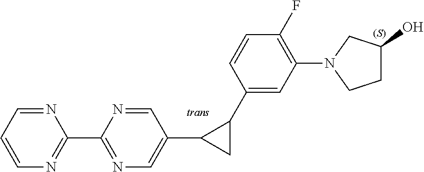 | trans-(3S)-1-(5-(2-([2,2'- bipyrimidin]-5- yl)cyclopropyl)-2- fluorophenyl)pyrrolidin-3-ol (single diastereomer II) | 0.23 |
| 67 | 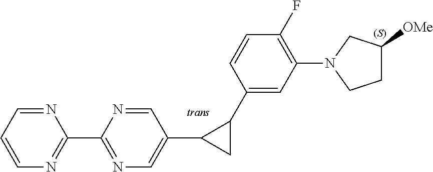 | trans-5-(2-(4-fluoro-3-((S)-3- methoxypyrrolidin-1- yl)phenyl)cyclopropyl)-2,2'- bipyrimidine (single diastereomer I) | 0.18 |
| 68 | 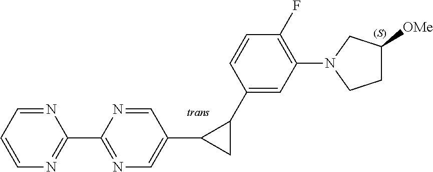 | trans-5-(2-(4-fluoro-3-((S)-3- methoxypyrrolidin-1- yl)phenyl)cyclopropyl)-2,2'- bipyrimidine (single diastereomer II) | 0.085 |
| 69 | 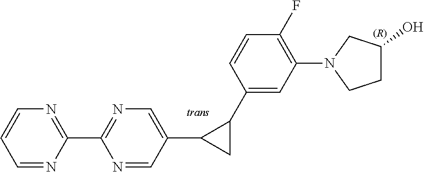 | trans-(3R)-1-(5-(2-([2,2- bipyrimidin]-5- yl)cyclopropyl)-2- fluorophenyl)pyrrolidin-3-ol (single diastereomer I) | 0.17 |
| 70 | 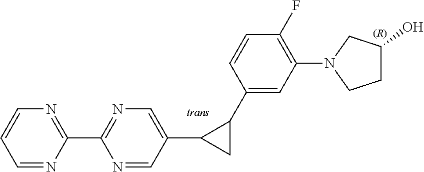 | trans-(3R)-1-(5-(2-([2,2- bipyrimidin]-5- yl)cyclopropyl)-2- fluorophenyl)pyrrolidin-3-ol (single diastereomer II) | 0.27 |
| 71 | 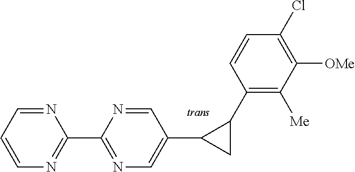 [SMILES: COc1c(Cl)ccc(C2CC2c2cnc(-c3ncccn3)nc2)c1C] | trans-5-(2-(4-chloro-3- methoxy-2- methylphenyl)cyclopropyl)- 2,2'-bipyrimidine (single enantiomer I) | 0.2 |
| 72 | 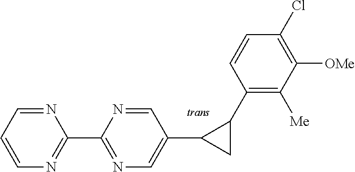 [SMILES: COc1c(Cl)ccc(C2CC2c2cnc(-c3ncccn3)nc2)c1C] | trans-5-(2-(4-chloro-3- methoxy-2- methylphenyl)cyclopropyl)- 2,2'-bipyrimidine (single enantiomer II) | 0.089 |
| 73 | 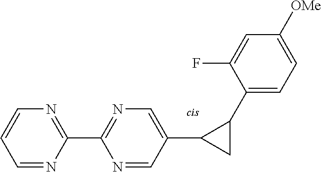 [SMILES: COc1ccc(C2CC2c2cnc(-c3ncccn3)nc2)c(F)c1] | cis-5-(2-(2-fluoro-4- methoxyphenyl)cyclopropyl)- 2,2-bipyrimidine | 9 |
| 74 | 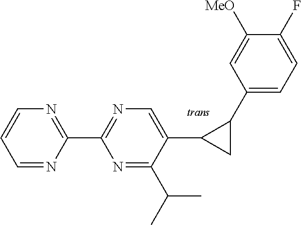 [SMILES: COc1cc(C2CC2c2cnc(-c3ncccn3)nc2C(C)C)ccc1F] | trans-5-(2-(4-fluoro-3- methoxyphenyl)cyclopropyl)- 4-isopropyl-2,2'- bipyrimidine (single enantiomer I) | 10 |
| 75 | 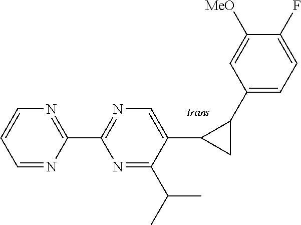 [SMILES: COc1cc(C2CC2c2cnc(-c3ncccn3)nc2C(C)C)ccc1F] | trans-5-(2-(4-fluoro-3- methoxyphenyl)cyclopropyl)- 4-isopropyl-2,2'- bipyrimidine (single enantiomer II) | 2 |
| 76 | 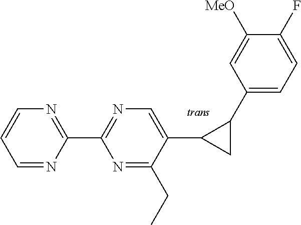 [SMILES: CCc1nc(-c2ncccn2)ncc1C1CC1c1ccc(F)c(OC)c1] | trans-4-ethyl-5-(2-(4-fluoro-3- methoxyphenyl)cyclopropyl)- 2,2'-bipyrimidine (single enantiomer I) | 2 |
| 77 | 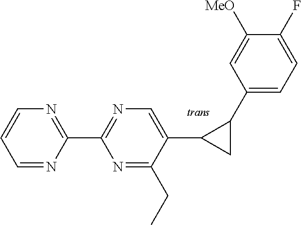 [SMILES: CCc1nc(-c2ncccn2)ncc1C1CC1c1ccc(F)c(OC)c1] | trans-4-ethyl-5-(2-(4-fluoro-3- methoxyphenyl)cyclopropyl)- 2,2'-bipyrimidine (single enantiomer II) | 5 |
| 78 | 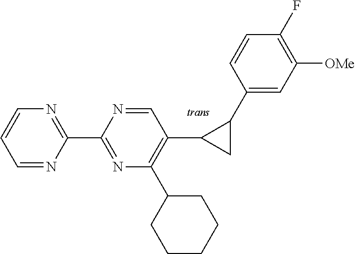 [SMILES: COc1cc(C2CC2c2cnc(-c3ncccn3)nc2C2CCCCC2)ccc1F] | trans-4-cyclohexyl-5-(2-(4- fluoro-3- methoxyphenyl)cyclopropyl)- 2,2'-bipyrimidine (single enantiomer I) | 11 |
| 79 | 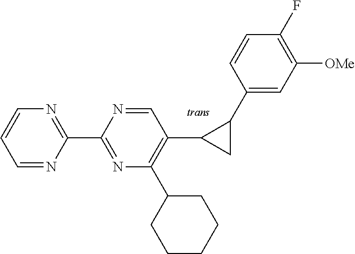 [SMILES: COc1cc(C2CC2c2cnc(-c3ncccn3)nc2C2CCCCC2)ccc1F] | trans-4-cyclohexyl-5-(2-(4- fluoro-3- methoxyphenyl)cyclopropyl)- 2,2'-bipyrimidine (single enantiomer II) | 0.22 |
| 80 | 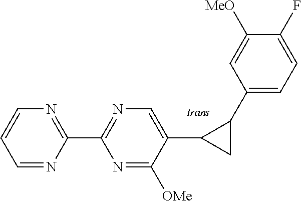 [SMILES: COc1cc(C2CC2c2cnc(-c3ncccn3)nc2OC)ccc1F] | trans-5-(2-(4-fluoro-3- methoxyphenyl)cyclopropyl)- 4-methoxy-2,2'- bipyrimidine (single enantiomer I) | 0.1 |
| 81 | 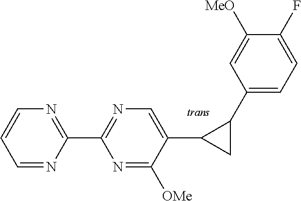 [SMILES: COc1cc(C2CC2c2cnc(-c3ncccn3)nc2OC)ccc1F] | trans-5-(2-(4-fluoro-3- methoxyphenyl)cyclopropyl)- 4-methoxy-2,2'- bipyrimidine (single enantiomer II) | 0.23 |
| 82 | 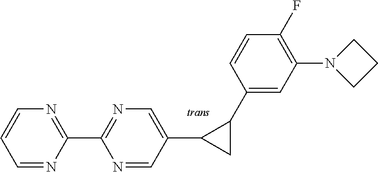 [SMILES: Fc1ccc(C2CC2c2cnc(-c3ncccn3)nc2)cc1N1CCC1] | trans-5-(2-(3-(azetidin-1-yl)- 4-fluorophenyl)cyclopropyl)- 2,2'-bipyrimidine (single enantiomer I) | 0.11 |
| 83 | 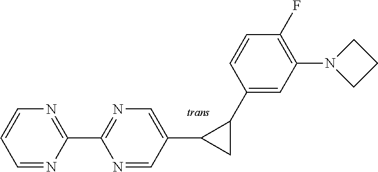 [SMILES: Fc1ccc(C2CC2c2cnc(-c3ncccn3)nc2)cc1N1CCC1] | trans-5-(2-(3-(azetidin-1-yl)- 4-fluorophenyl)cyclopropyl)- 2,2'-bipyrimidine (single enantiomer II) | 0.035 |
| 84 | 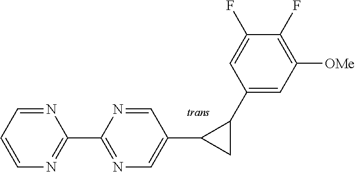 [SMILES: COc1cc(C2CC2c2cnc(-c3ncccn3)nc2)cc(F)c1F] | trans-5-(2-(3,4-difluoro-5- methoxyphenyl)cyclopropyl)- 2,2'-bipyrimidine (single enantiomer I) | 0.24 |
| 85 | 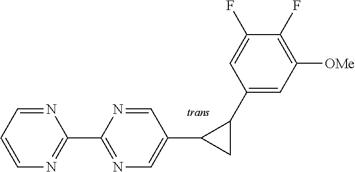 [SMILES: COc1cc(C2CC2c2cnc(-c3ncccn3)nc2)cc(F)c1F] | trans-5-(2-(3,4-difluoro-5- methoxyphenyl)cyclopropyl)- 2,2'-bipyrimidine (single enantiomer II) | 0.029 |
| 86 | 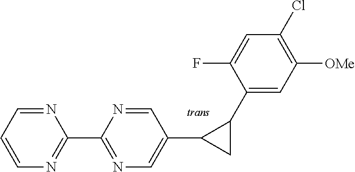 [SMILES: COc1cc(C2CC2c2cnc(-c3ncccn3)nc2)c(F)cc1Cl] | trans-5-(2-(4-chloro-2- fluoro-5- methoxyphenyl)cyclopropyl)- 2,2'-bipyrimidine (single enantiomer I) | 0.13 |
| 87 | 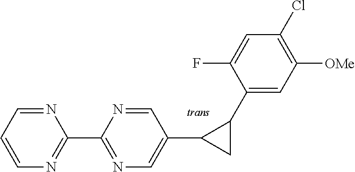 [SMILES: COc1cc(C2CC2c2cnc(-c3ncccn3)nc2)c(F)cc1Cl] | trans-5-(2-(4-chloro-2- fluoro-5- methoxyphenyl)cyclopropyl)- 2,2'-bipyrimidine (single enantiomer II) | 0.14 |
| 88 | 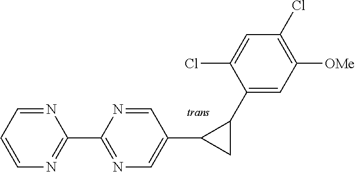 [SMILES: COc1cc(C2CC2c2cnc(-c3ncccn3)nc2)c(Cl)cc1Cl] | trans-5-(2-(2,4-dichloro-5- methoxyphenyl)cyclopropyl)- 2,2'-bipyrimidine (single enantiomer I) | 0.084 |
| 89 | 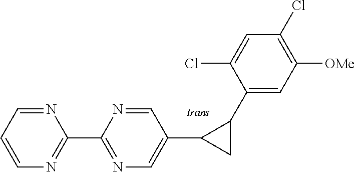 [SMILES: COc1cc(C2CC2c2cnc(-c3ncccn3)nc2)c(Cl)cc1Cl] | trans-5-(2-(2,4-dichloro-5- methoxyphenyl)cyclopropyl)- 2,2'-bipyrimidine (single enantiomer II) | 0.61 |
| 90 | 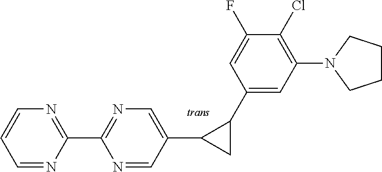 [SMILES: Fc1cc(C2CC2c2cnc(-c3ncccn3)nc2)cc(N2CCCC2)c1Cl] | trans-5-(2-(4-chloro-3- fluoro-5-(pyrrolidin-1- yl)phenyl)cyclopropyl)-2,2'- bipyrimidine (single enantiomer I) | 0.028 |
| 91 | 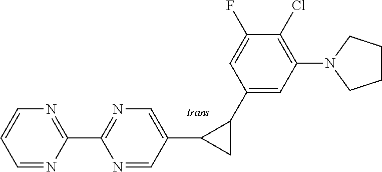 [SMILES: Fc1cc(C2CC2c2cnc(-c3ncccn3)nc2)cc(N2CCCC2)c1Cl] | trans-5-(2-(4-chloro-3- fluoro-5-(pyrrolidin-1- yl)phenyl)cyclopropyl)-2,2'- bipyrimidine (single enantiomer II) | 0.014 |
| 92 | 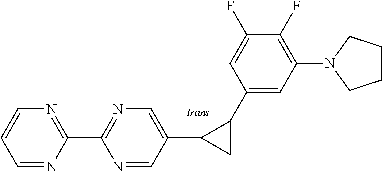 [SMILES: Fc1cc(C2CC2c2cnc(-c3ncccn3)nc2)cc(N2CCCC2)c1F] | tran-5-(2-(3,4-difluoro-5- (pyrrolidin-1- yl)phenyl)cyclopropyl)-2,2'- bipyrimidine (single enantiomer I) | 0.024 |
| 93 | 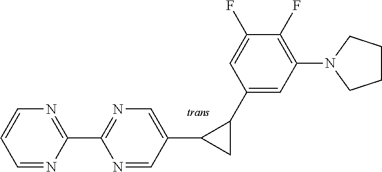 [SMILES: Fc1cc(C2CC2c2cnc(-c3ncccn3)nc2)cc(N2CCCC2)c1F] | tran-5-(2-(3,4-difluoro-5- (pyrrolidin-1- yl)phenyl)cyclopropyl)-2,2'- bipyrimidine (single enantiomer II) | 0.019 |
| 94 | 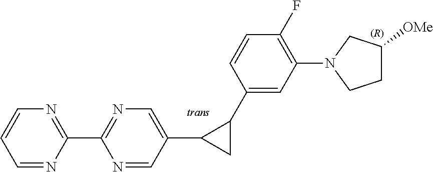 | trans-5-(2-(4-fluoro-3-((R)- 3-methoxypyrrolidin-1- yl)phenyl)cyclopropyl)-2,2'- bipyrimidine (single diastereomer I) | 0.27 |
| 95 | 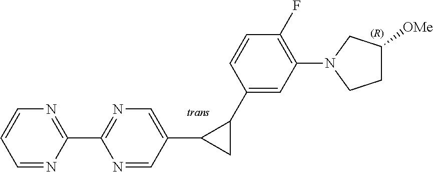 | trans-5-(2-(4-fluoro-3-((R)- 3-methoxypyrrolidin-1- yl)phenyl)cyclopropyl)-2,2'- bipyrimidine (single diastereomer II) | 0.17 |
| 96 | 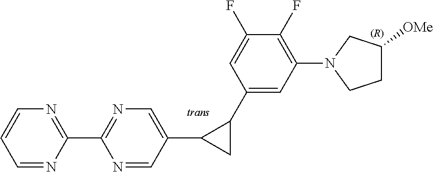 | trans-5-(2-(3,4-difluoro-5- ((R)-3-methoxypyrrolidin-1- yl)phenyl)cyclopropyl)-2,2'- bipyrimidine (single diastereomer I) | 0.093 |
| 97 | 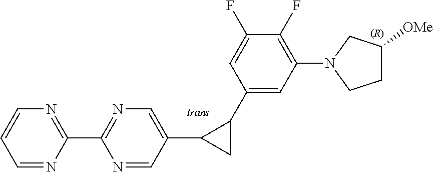 | trans-5-(2-(3,4-difluoro-5- ((R)-3-methoxypyrrolidin-1- yl)phenyl)cyclopropyl)-2,2'- bipyrimidine (single diastereomer II) | 0.014 |
| 98 | 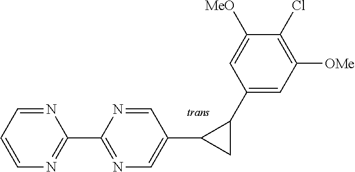 [SMILES: COc1cc(C2CC2c2cnc(-c3ncccn3)nc2)cc(OC)c1Cl] | trans-5-(2-(4-chloro-3,5- dimethoxyphenyl)cyclopropyl)- 2,2'-bipyrimidine (single enantiomer I) | 0.35 |
| 99 | 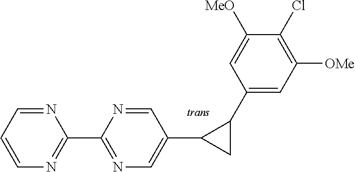 [SMILES: COc1cc(C2CC2c2cnc(-c3ncccn3)nc2)cc(OC)c1Cl] | trans-5-(2-(4-chloro-3,5- dimethoxyphenyl)cyclopropyl)- 2,2'-bipyrimidine (single enantiomer II) | 3 |
| 100 | 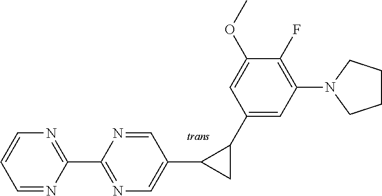 [SMILES: COc1cc(C2CC2c2cnc(-c3ncccn3)nc2)cc(N2CCCC2)c1F] | trans-5-(2-(4-fluoro-3- methoxy-5-(pyrrolidin-1- yl)phenyl)cyclopropyl)-2,2'- bipyrimidine (single enantiomer I) | 0.061 |
| 101 | 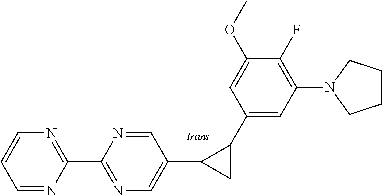 [SMILES: COc1cc(C2CC2c2cnc(-c3ncccn3)nc2)cc(N2CCCC2)c1F] | trans-5-(2-(4-fluoro-3- methoxy-5-(pyrrolidin-1- yl)phenyl)cyclopropyl)-2,2'- bipyrimidine (single enantiomer II) | 0.8 |
| 102 | 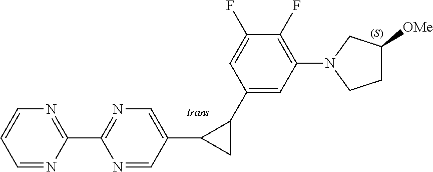 | trans-5-(2-(3,4-difluoro-5- ((S)-3-methoxypyrrolidin-1- yl)phenyl)cyclopropyl)-2,2'- bipyrimidine (single diastereomer I) | 0.029 |
| 103 | 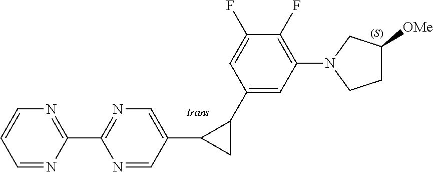 | trans-5-(2-(3,4-difluoro-5- ((S)-3-methoxypyrrolidin-1- yl)phenyl)cyclopropyl)-2,2'- bipyrimidine (single diastereomer II) | 0.008 |
| 104 | 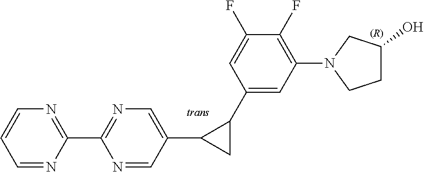 | trans-(3R)-1-(5-(2-([2,2- bipyrimidin]-5- yl)cyclopropyl)-2,3- difluorophenyl)pyrrolidin-3-ol (single diastereomer I) | 0.078 |
| 105 | 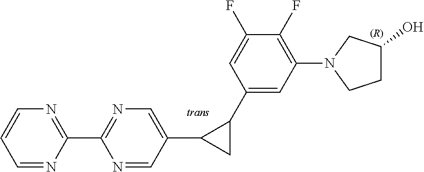 | trans-(3R)-1-(5-(2-([2,2- bipyrimidin]-5- yl)cyclopropyl)-2,3- difluorophenyl)pyrrolidin-3-ol (single diastereomer II) | 0.097 |
| 106 | 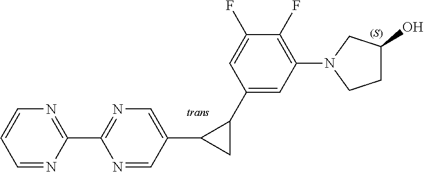 | trans-(2S)-1-(5-(2-([2,2'- bipyrimidin]-5- yl)cyclopropyl)-2,3- difluorophenyl)pyrrolidin-3- ol (single diastereomer I) | 0.085 |
| 107 | 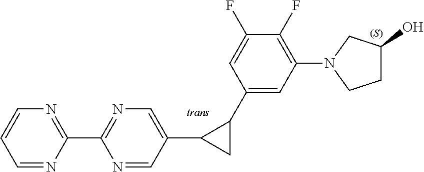 | trans-(2S)-1-(5-(2-([2,2'- bipyrimidin]-5- yl)cyclopropyl)-2,3- difluorophenyl)pyrrolidin-3- ol (single diastereomer II) | 0.021 |
| 108 | 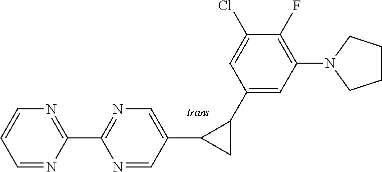 [SMILES: Fc1c(Cl)cc(C2CC2c2cnc(-c3ncccn3)nc2)cc1N1CCCC1] | trans-5-(2-(3-chloro-4- fluoro-5-(pyrrolidin-1- yl)phenyl)cyclopropyl)-2,2'- bipyrimidine (single enantiomer I) | 0.078 |
| 109 | 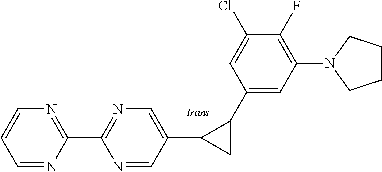 [SMILES: Fc1c(Cl)cc(C2CC2c2cnc(-c3ncccn3)nc2)cc1N1CCCC1] | trans-5-(2-(3-chloro-4- fluoro-5-(pyrrolidin-1- yl)phenyl)cyclopropyl)-2,2'- bipyrimidine (single enantiomer II) | 0.025 |
| 110 | 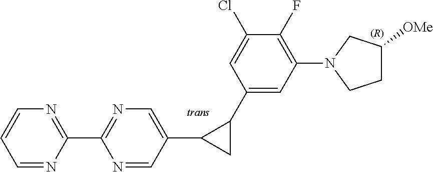 | trans-5-(2-(3-chloro-4- fluoro-5-((R)-3- methoxypyrrolidin-1- yl)phenyl)cyclopropyl)-2,2'- bipyrimidine (single diastereomer I) | 0.19 |
| 111 | 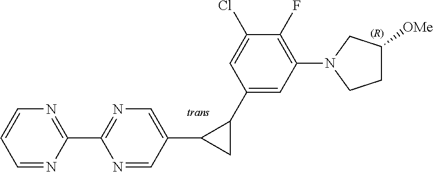 | trans-5-(2-(3-chloro-4- fluoro-5-((R)-3- methoxypyrrolidin-1- yl)phenyl)cyclopropyl)-2,2'- bipyrimidine (single diastereomer II) | 0.12 |
| 112 | 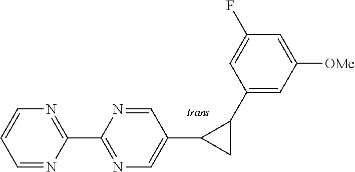 [SMILES: COc1cc(F)cc(C2CC2c2cnc(-c3ncccn3)nc2)c1] | trans-5-(2-(3-fluoro-5- methoxyphenyl)cyclopropyl)- 2,2'-bipyrimidine (single enantiomer I) | 0.5 |
| 113 | 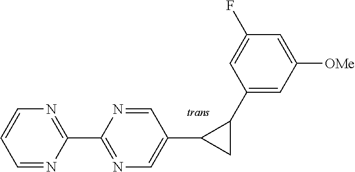 [SMILES: COc1cc(F)cc(C2CC2c2cnc(-c3ncccn3)nc2)c1] | trans-5-(2-(3-fluoro-5- methoxyphenyl)cyclopropyl)- 2,2'-bipyrimidine (single enantiomer II) | 0.026 |
| 114 | 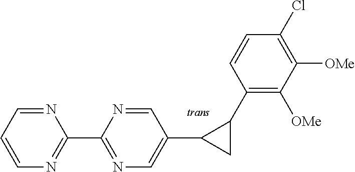 [SMILES: COc1c(Cl)ccc(C2CC2c2cnc(-c3ncccn3)nc2)c1OC] | trans-5-(2-(4-chloro-2,3- dimethoxyphenyl)cyclopropyl)- 2,2'-bipyrimidine (single enantiomer I) | 3 |
| 115 | 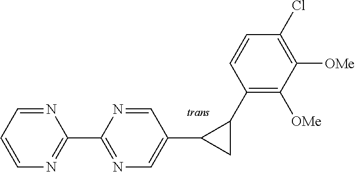 [SMILES: COc1c(Cl)ccc(C2CC2c2cnc(-c3ncccn3)nc2)c1OC] | trans-5-(2-(4-chloro-2,3- dimethoxyphenyl)cyclopropyl)- 2,2'-bipyrimidine (single enantiomer II) | 0.32 |
| 116 | 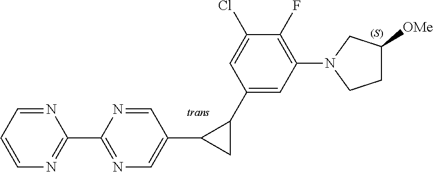 | trans-5-(2-(3-chloro-4- fluoro-5-((S)-3- methoxypyrrolidin-1- yl)phenyl)cyclopropyl)-2,2'- bipyrimidine (single diastereomer I) | 0.09 |
| 117 | 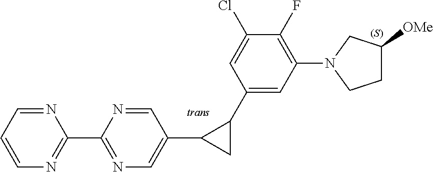 | trans-5-(2-(3-chloro-4- fluoro-5-((S)-3- methoxypyrrolidin-1- yl)phenyl)cyclopropyl)-2,2'- bipyrimidine (single diastereomer II) | 0.011 |
| 118 | 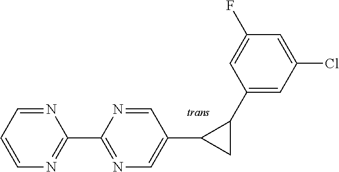 [SMILES: Fc1cc(Cl)cc(C2CC2c2cnc(-c3ncccn3)nc2)c1] | trans-5-(2-(3-chloro-5- fluorophenyl)cyclopropyl)- 2,2'-bipyrimidine (single enantiomer I) | 2 |
| 119 | 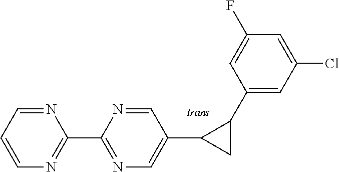 [SMILES: Fc1cc(Cl)cc(C2CC2c2cnc(-c3ncccn3)nc2)c1] | trans-5-(2-(3-chloro-5- fluorophenyl)cyclopropyl)- 2,2'-bipyrimidine (single enantiomer II) | 0.33 |
| 120 | 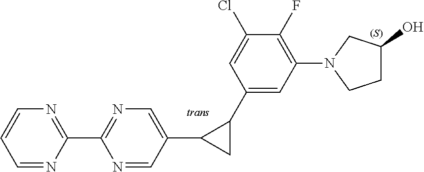 | trans-(3S)-1-(5-(2-([2,2'- bipyrimidin]-5- yl)cyclopropyl)-3-chloro-2- fluorophenyl)pyrrolidin-3-ol (single diastereomer I) | 0.099 |
| 121 | 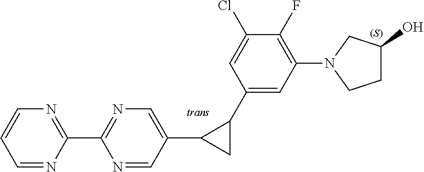 | trans-(3S)-1-(5-(2-([2,2'- bipyrimidin]-5- yl)cyclopropyl)-3-chloro-2- fluorophenyl)pyrrolidin-3-ol (single diastereomer II) | 0.011 |
| 122 | 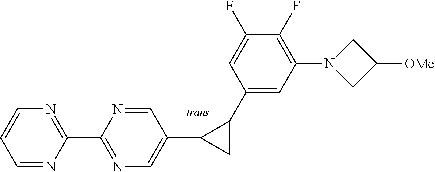 [SMILES: COC1CN(c2cc(C3CC3c3cnc(-c4ncccn4)nc3)cc(F)c2F)C1] | trans-5-(2-(3,4-difluoro-5- (3-methoxyazetidin-1- yl)phenyl)cyclopropyl)-2,2'- bipyrimidine (single enantiomer I) | 0.061 |
| 123 | 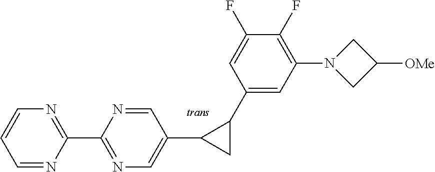 [SMILES: COC1CN(c2cc(C3CC3c3cnc(-c4ncccn4)nc3)cc(F)c2F)C1] | trans-5-(2-(3,4-difluoro-5- (3-methoxyazetidin-1- yl)phenyl)cyclopropyl)-2,2'- bipyrimidine (single enantiomer II) | 0.012 |
| 124 | 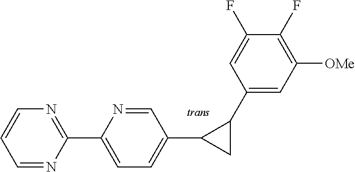 [SMILES: COc1cc(C2CC2c2ccc(-c3ncccn3)nc2)cc(F)c1F] | trans-2-(5-(2-(3,4-difluoro-5- methoxyphenyl)cyclopropyl) pyridin-2-yl)pyrimidine (single enantiomer I) | 0.29 |
| 125 | 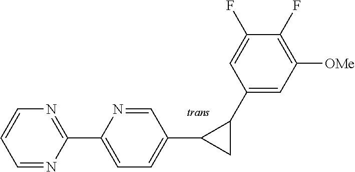 [SMILES: COc1cc(C2CC2c2ccc(-c3ncccn3)nc2)cc(F)c1F] | trans-2-(5-(2-(3,4-difluoro-5- methoxyphenyl)cyclopropyl) pyridin-2-yl)pyrimidine (single enantiomer II) | 0.004 |
| 126 | 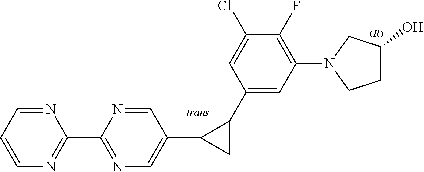 | trans-(3R)-1-(5-(2-([2,2'- bipyrimidin]-5- yl)cyclopropyl)-3-chloro-2- fluorophenyl)pyrrolidin-3-ol (single diastereomer I) | 0.12 |
| 127 | 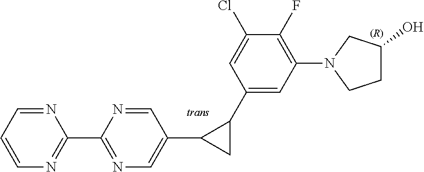 | trans-(3R)-1-(5-(2-([2,2'- bipyrimidin]-5- yl)cyclopropyl)-3-chloro-2- fluorophenyl)pyrrolidin-3-ol (single diastereomer II) | 0.11 |
| 128 | 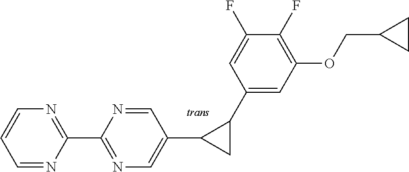 [SMILES: Fc1cc(C2CC2c2cnc(-c3ncccn3)nc2)cc(OCC2CC2)c1F] | trans-5-(2-(3- (cyclopropylmethoxy)-4,5- difluorophenyl)cyclopropyl)- 2,2'-bipyrimidine (single enantiomer I) | 0.42 |
| 129 | 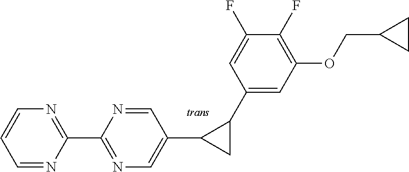 [SMILES: Fc1cc(C2CC2c2cnc(-c3ncccn3)nc2)cc(OCC2CC2)c1F] | trans-5-(2-(3- (cyclopropylmethoxy)-4,5- difluorophenyl)cyclopropyl)- 2,2'-bipyrimidine (single enantiomer II) | 0.018 |
| 130 | 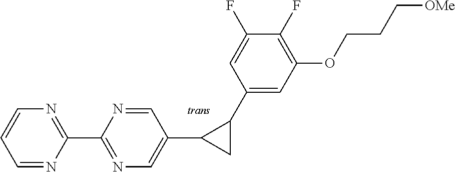 [SMILES: COCCCOc1cc(C2CC2c2cnc(-c3ncccn3)nc2)cc(F)c1F] | trans-5-(2-(3,4-difluoro-5-(3- methoxypropoxy)phenyl) cyclopropyl)-2,2'-bipyrimidine (single enantiomer I) | 0.26 |
| 131 | 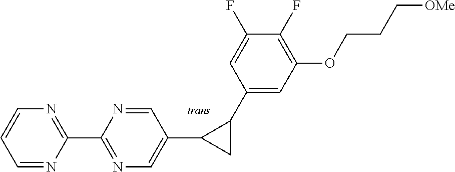 [SMILES: COCCCOc1cc(C2CC2c2cnc(-c3ncccn3)nc2)cc(F)c1F] | trans-5-(2-(3,4-difluoro-5-(3- methoxypropoxy)phenyl) cyclopropyl)-2,2'-bipyrimidine (single enantiomer II) | 0.015 |
| 132 | 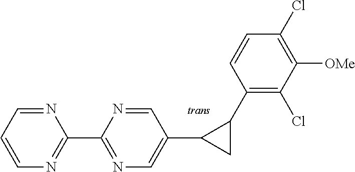 [SMILES: COc1c(Cl)ccc(C2CC2c2cnc(-c3ncccn3)nc2)c1Cl] | trans-5-(2-(2,4-dichloro-3- methoxyphenyl)cyclopropyl)- 2,2'-bipyrimidine (single enantiomer I) | 0.12 |
| 133 | 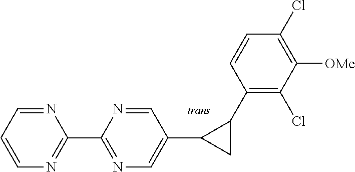 [SMILES: COc1c(Cl)ccc(C2CC2c2cnc(-c3ncccn3)nc2)c1Cl] | trans-5-(2-(2,4-dichloro-3- methoxyphenyl)cyclopropyl)- 2,2'-bipyrimidine (single enantiomer II) | 0.13 |
| 134 | 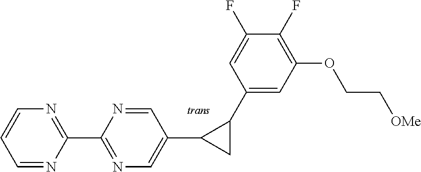 [SMILES: COCCOc1cc(C2CC2c2cnc(-c3ncccn3)nc2)cc(F)c1F] | trans-5-(2-(3,4-difluoro-5-(2- methoxyethoxy)phenyl) cyclopropyl)-2,2'-bipyrimidine (single enantiomer I) | 0.3 |
| 135 | 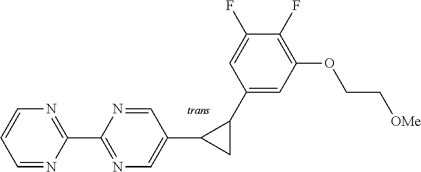 [SMILES: COCCOc1cc(C2CC2c2cnc(-c3ncccn3)nc2)cc(F)c1F] | trans-5-(2-(3,4-difluoro-5-(2- methoxyethoxy)phenyl) cyclopropyl)-2,2'-bipyrimidine (single enantiomer II) | 0.022 |
| 136 | 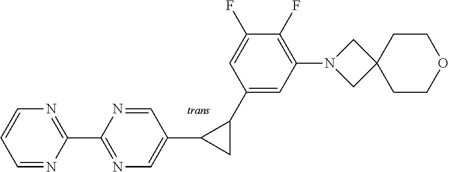 [SMILES: Fc1cc(C2CC2c2cnc(-c3ncccn3)nc2)cc(N2CC3(CCOCC3)C2)c1F] | trans-2-(5-(2-([2,2'- bipyrimidin]-5- yl)cyclopropyl)-2,3- difluorophenyl)-7-oxa-2- azaspiro[3.5]nonane (single enantiomer I) | 0.92 |
| 137 | 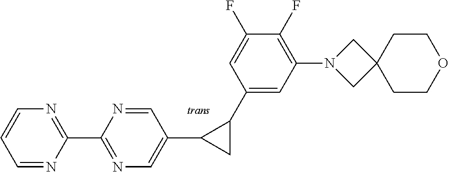 [SMILES: Fc1cc(C2CC2c2cnc(-c3ncccn3)nc2)cc(N2CC3(CCOCC3)C2)c1F] | trans-2-(5-(2-([2,2'- bipyrimidin]-5- yl)cyclopropyl)-2,3- difluorophenyl)-7-oxa-2- azaspiro[3.5]nonane (single enantiomer II) | 0.074 |
| 138 | 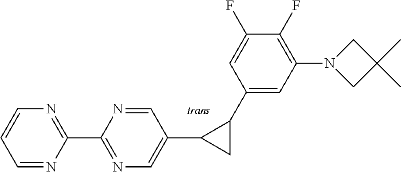 [SMILES: CC1(C)CN(c2cc(C3CC3c3cnc(-c4ncccn4)nc3)cc(F)c2F)C1] | trans-5-(2-(3-(3,3- dimethylazetidin-1-yl)-4,5- difluorophenyl)cyclopropyl)- 2,2'-bipyrimidine (single enantiomer I) | 1 |
| 139 | 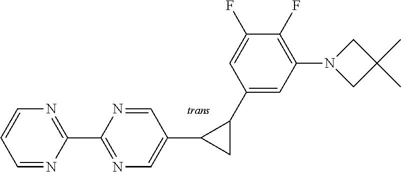 [SMILES: CC1(C)CN(c2cc(C3CC3c3cnc(-c4ncccn4)nc3)cc(F)c2F)C1] | trans-5-(2-(3-(3,3- dimethylazetidin-1-yl)-4,5- difluorophenyl)cyclopropyl)- 2,2'-bipyrimidine (single enantiomer II) | 0.088 |
| 140 | 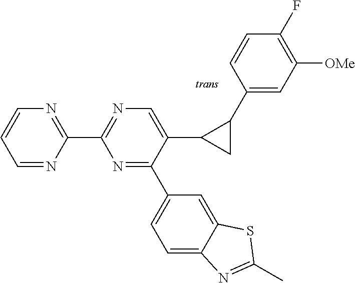 [SMILES: COc1cc(C2CC2c2cnc(-c3ncccn3)nc2-c2ccc3nc(C)sc3c2)ccc1F] | trans-6-(5-(2-(4-fluoro-3- methoxyphenyl)cyclopropyl)- [2,2'-bipyrimidin]-4-yl)-2- methylbenzo[d]thiazole (single enantiomer I) | 37 |
| 141 | 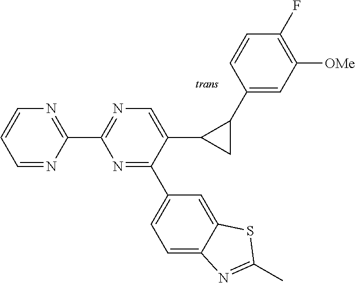 [SMILES: COc1cc(C2CC2c2cnc(-c3ncccn3)nc2-c2ccc3nc(C)sc3c2)ccc1F] | trans-6-(5-(2-(4-fluoro-3- methoxyphenyl)cyclopropyl)- [2,2'-bipyrimidin]-4-yl)-2- methylbenzo[d]thiazole (single enantiomer II) | 1.2 |
| 142 | 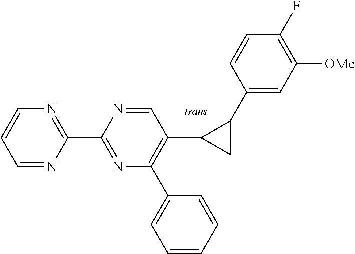 [SMILES: COc1cc(C2CC2c2cnc(-c3ncccn3)nc2-c2ccccc2)ccc1F] | trans-5-(2-(4-Fluoro-3- methoxyphenyl)cyclopropyl)- 4-phenyl-2,2'-bipyrimidine (single enantiomer I) | 5 |
| 143 | 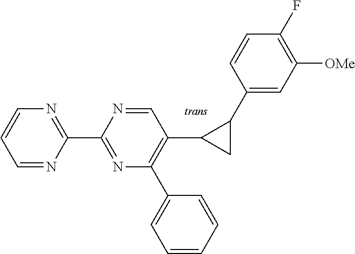 [SMILES: COc1cc(C2CC2c2cnc(-c3ncccn3)nc2-c2ccccc2)ccc1F] | trans-5-(2-(4-Fluoro-3- methoxyphenyl)cyclopropyl)- 4-phenyl-2,2'-bipyrimidine (single enantiomer II) | 0.12 |
| 144 | 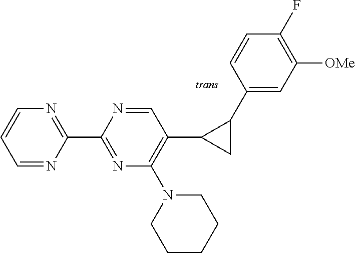 [SMILES: COc1cc(C2CC2c2cnc(-c3ncccn3)nc2N2CCCCC2)ccc1F] | trans-5-(2-(4-fluoro-3- methoxyphenyl)cyclopropyl)- 4-(piperidin-1-yl)-2,2'- bipyrimidine (single enantiomer I) | 25 |
| 145 | 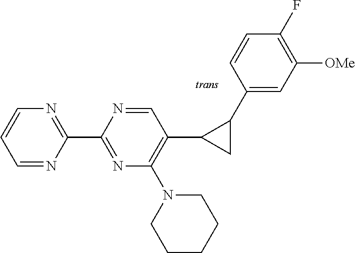 [SMILES: COc1cc(C2CC2c2cnc(-c3ncccn3)nc2N2CCCCC2)ccc1F] | trans-5-(2-(4-fluoro-3- methoxyphenyl)cyclopropyl)- 4-(piperidin-1-yl)-2,2'- bipyrimidine (single enantiomer II) | 0.7 |
| 146 | 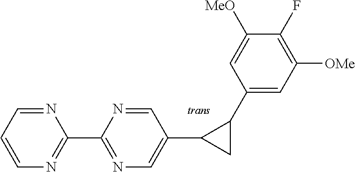 [SMILES: COc1cc(C2CC2c2cnc(-c3ncccn3)nc2)cc(OC)c1F] | trans-5-(2-(4-fluoro-3,5- dimethoxyphenyl)cyclopropyl)- 2,2'-bipyrimidine (single enantiomer I) | 0.11 |
| 147 | 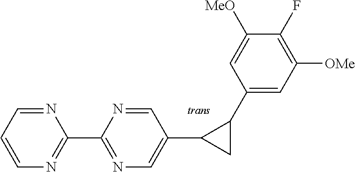 [SMILES: COc1cc(C2CC2c2cnc(-c3ncccn3)nc2)cc(OC)c1F] | trans-5-(2-(4-fluoro-3,5- dimethoxyphenyl)cyclopropyl)- 2,2'-bipyrimidine (single enantiomer II) | 10 |
| 148 | 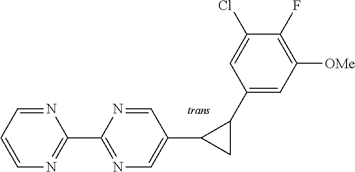 [SMILES: COc1cc(C2CC2c2cnc(-c3ncccn3)nc2)cc(Cl)c1F] | trans-5-(2-(3-chloro-4- fluoro-5- methoxyphenyl)cyclopropyl)- 2,2'-bipyrimidine (single enantiomer I) | 1 |
| 149 | 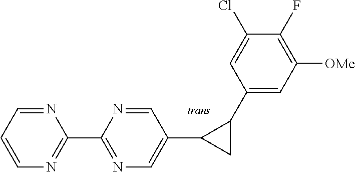 [SMILES: COc1cc(C2CC2c2cnc(-c3ncccn3)nc2)cc(Cl)c1F] | trans-5-(2-(3-chloro-4- fluoro-5- methoxyphenyl)cyclopropyl)- 2,2'-bipyrimidine (single enantiomer II) | 0.062 |
| 150 | 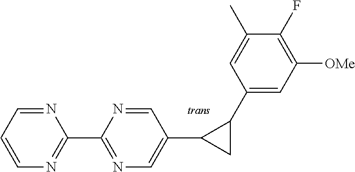 [SMILES: COc1cc(C2CC2c2cnc(-c3ncccn3)nc2)cc(C)c1F] | trans-5-(2-(4-fluoro-3- methoxy-5- methylphenyl)cyclopropyl)- 2,2'-bipyrimidine (single enantiomer I) | 1 |
| 151 | 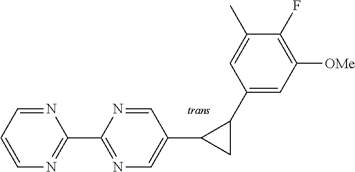 [SMILES: COc1cc(C2CC2c2cnc(-c3ncccn3)nc2)cc(C)c1F] | trans-5-(2-(4-fluoro-3- methoxy-5- methylphenyl)cyclopropyl)- 2,2'-bipyrimidine (single enantiomer II) | 0.081 |
| 152 | 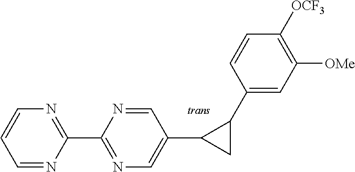 [SMILES: COc1cc(C2CC2c2cnc(-c3ncccn3)nc2)ccc1OC(F)(F)F] | trans-5-(2-(3-methoxy-4- (trifluoromethoxy)phenyl) cyclopropyl)-2,2'-bipyrimidine | 0.7 |
| 153 | 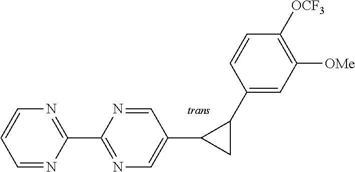 [SMILES: COc1cc(C2CC2c2cnc(-c3ncccn3)nc2)ccc1OC(F)(F)F] | trans-5-(2-(3-methoxy-4- (trifluoromethyl)phenyl) cyclopropyl)-2,2'-bipyrimidine | 0.19 |
| 154 | 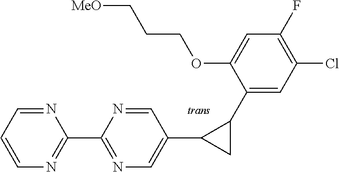 [SMILES: COCCCOc1cc(F)c(Cl)cc1C1CC1c1cnc(-c2ncccn2)nc1] | trans-5-(2-(5-chloro-4- fluoro-2-(3- methoxypropoxy)phenyl) cyclopropyl)-2,2'-bipyrimidine (single enantiomer I) | 0.3 |
| 155 | 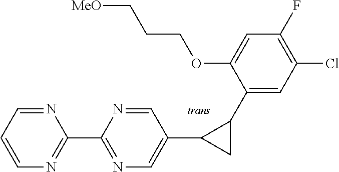 [SMILES: COCCCOc1cc(F)c(Cl)cc1C1CC1c1cnc(-c2ncccn2)nc1] | trans-5-(2-(5-chloro-4- fluoro-2-(3- methoxypropoxy)phenyl) cyclopropyl)-2,2'-bipyrimidine (single enantiomer II) | 3 |
| 156 | 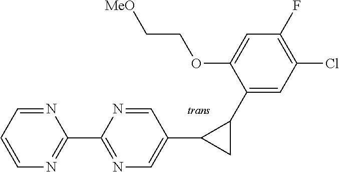 [SMILES: COCCOc1cc(F)c(Cl)cc1C1CC1c1cnc(-c2ncccn2)nc1] | trans-5-(2-(5-chloro-4- fluoro-2-(2- methoxyethoxy)phenyl) cyclopropyl)-2,2'-bipyrimidine (single enantiomer I) | 0.19 |
| 157 | 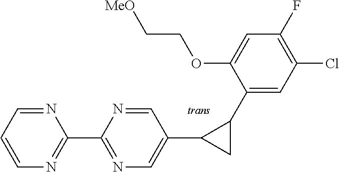 [SMILES: COCCOc1cc(F)c(Cl)cc1C1CC1c1cnc(-c2ncccn2)nc1] | trans-5-(2-(5-chloro-4- fluoro-2-(2- methoxyethoxy)phenyl) cyclopropyl)-2,2'-bipyrimidine (single enantiomer II) | 49 |
| 158 | 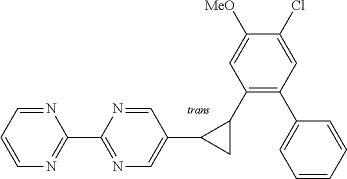 [SMILES: COc1cc(C2CC2c2cnc(-c3ncccn3)nc2)c(-c2ccccc2)cc1Cl] | trans-5-(2-(5-chloro-4- methoxy-[1,1'-biphenyl]-2- yl)cyclopropyl)-2,2'- bipyrimidine (single enantiomer I) | 3 |
| 159 | 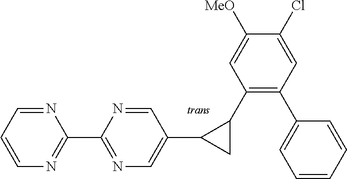 [SMILES: COc1cc(C2CC2c2cnc(-c3ncccn3)nc2)c(-c2ccccc2)cc1Cl] | trans-5-(2-(5-chloro-4- methoxy-[1,1'-biphenyl]-2- yl)cyclopropyl)-2,2'- bipyrimidine (single enantiomer II) | 49 |
| 160 | 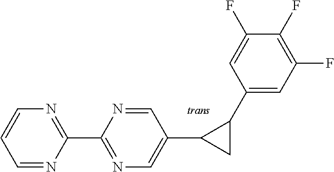 [SMILES: Fc1cc(C2CC2c2cnc(-c3ncccn3)nc2)cc(F)c1F] | trans-5-(2-(3,4,5- trifluorophenyl)cyclopropyl)- 2,2'-bipyrimidine | 0.27 |
| 161 | 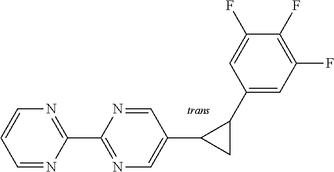 [SMILES: Fc1cc(C2CC2c2cnc(-c3ncccn3)nc2)cc(F)c1F] | trans-5-(2-(3,4,5- trifluorophenyl)cyclopropyl)- 2,2'-bipyrimidine (single enantiomer I) | 10 |
| 162 | 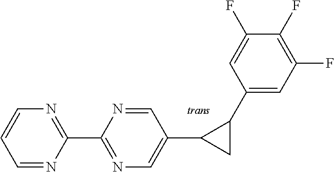 [SMILES: Fc1cc(C2CC2c2cnc(-c3ncccn3)nc2)cc(F)c1F] | trans-5-(2-(3,4,5- trifluorophenyl)cyclopropyl)- 2,2'-bipyrimidine (single enantiomer II) | 0.16 |
| 163 | 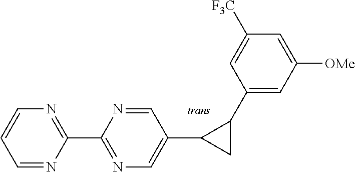 [SMILES: COc1cc(C2CC2c2cnc(-c3ncccn3)nc2)cc(C(F)(F)F)c1] | trans-5-(2-(3-methoxy-5- (trifluoromethyl)phenyl) cyclopropyl)-2,2'-bipyrimidine | 0.14 |
| 164 | 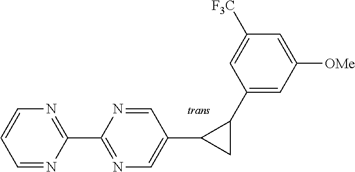 [SMILES: COc1cc(C2CC2c2cnc(-c3ncccn3)nc2)cc(C(F)(F)F)c1] | trans-5-(2-(3-methoxy-5- (trifluoromethoxy)phenyl) cyclopropyl)-2,2'-bipyrimidine | 0.48 |
| 165 | 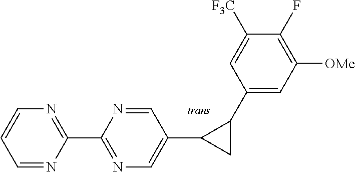 [SMILES: COc1cc(C2CC2c2cnc(-c3ncccn3)nc2)cc(C(F)(F)F)c1F] | trans-5-(2-(4-fluoro-3- methoxy-5- (trifluoromethyl)phenyl) cyclopropyl)-2,2'-bipyrimidine | 0.25 |
| 166 | 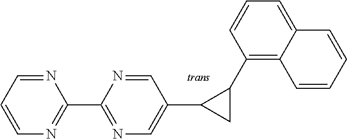 [SMILES: c1cnc(-c2ncc(C3CC3c3cccc4ccccc34)cn2)nc1] | trans-5-(2-(naphthalen-1- yl)cyclopropyl)-2,2'- bipyrimidine | 0.065 |
| 167 | 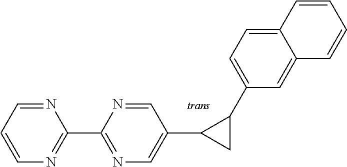 [SMILES: c1cnc(-c2ncc(C3CC3c3ccc4ccccc4c3)cn2)nc1] | trans-5-(2-(naphthalen-2- yl)cyclopropyl)-2,2'- bipyrimidine | 0.18 |
| 168 | 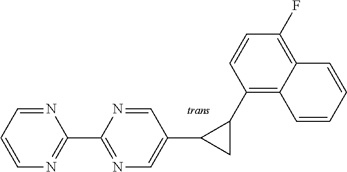 [SMILES: Fc1ccc(C2CC2c2cnc(-c3ncccn3)nc2)c2ccccc12] | trans-5-(2-(4- fluoronaphthalen-1- yl)cyclopropyl)-2,2'- bipyrimidine | 0.036 |
| 169 | 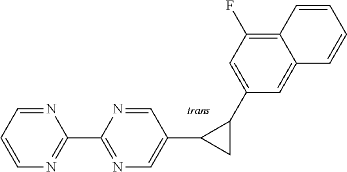 [SMILES: Fc1cc(C2CC2c2cnc(-c3ncccn3)nc2)cc2ccccc12] | trans-5-(2-(4- fluoronaphthalen-2- yl)cyclopropyl)-2,2'- bipyrimidine | 0.036 |
| 170 | 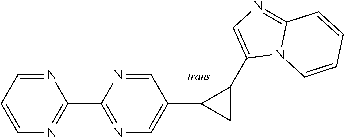 [SMILES: c1cnc(-c2ncc(C3CC3c3cnc4ccccn34)cn2)nc1] | trans-3-(2-([2,2'- bipyrimidin]-5- yl)cyclopropyl)imidazo[1,2- a]pyridine | 1 |
| 171 | 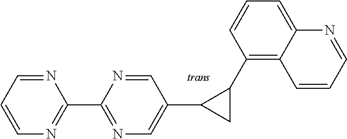 [SMILES: c1cnc(-c2ncc(C3CC3c3cccc4ncccc34)cn2)nc1] | trans-5-(2-([2,2'- bipyrimidin]-5- yl)cyclopropyl)quinoline | 0.38 |
| 172 | 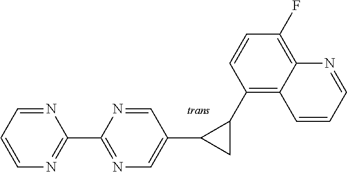 [SMILES: Fc1ccc(C2CC2c2cnc(-c3ncccn3)nc2)c2cccnc12] | trans-5-(2-([2,2'- bipyrimidin]-5- yl)cyclopropyl)-8- fluoroquinoline | 0.25 |
| 173 | 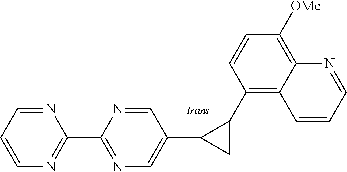 [SMILES: COc1ccc(C2CC2c2cnc(-c3ncccn3)nc2)c2cccnc12] | trans-5-(2-([2,2'- bipyrimidin]-5- yl)cyclopropyl)-8- methoxyquinoline | 0.54 |
| 174 | 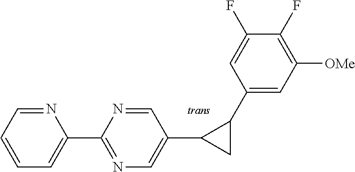 [SMILES: COc1cc(C2CC2c2cnc(-c3ccccn3)nc2)cc(F)c1F] | trans-5-(2-(3,4-difluoro-5- methoxyphenyl)cyclopropyl)- 2-(pyridin-2-yl)pyrimidine | 0.067 |
| 175 | 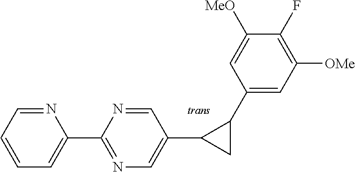 [SMILES: COc1cc(C2CC2c2cnc(-c3ccccn3)nc2)cc(OC)c1F] | trans-5-(2-(4-fluoro-3,5- dimethoxyphenyl)cyclopropyl)- 2-(pyridin-2-yl)pyrimidine | 0.35 |
| 176 | 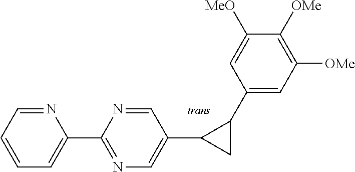 [SMILES: COc1cc(C2CC2c2cnc(-c3ccccn3)nc2)cc(OC)c1OC] | trans-2-(pyridin-2-yl)-5-(2- (3,4,5-trimethoxyphenyl) cyclopropyl)pyrimidine | 2 |
| 177 | 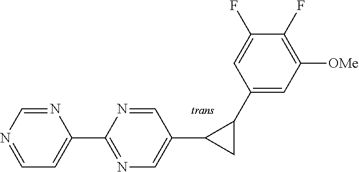 [SMILES: COc1cc(C2CC2c2cnc(-c3ccncn3)nc2)cc(F)c1F] | trans-5-(2-(3,4-difluoro-5- methoxyphenyl)cyclopropyl)- 2,4'-bipyrimidine | 0.36 |
| 178 | 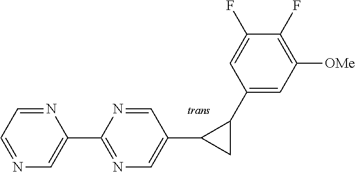 [SMILES: COc1cc(C2CC2c2cnc(-c3cnccn3)nc2)cc(F)c1F] | trans-5-(2-(3,4-difluoro-5- methoxyphenyl)cyclopropyl)- 2-(pyrazin-2-yl)pyrimidine | 0.46 |
| 179 | 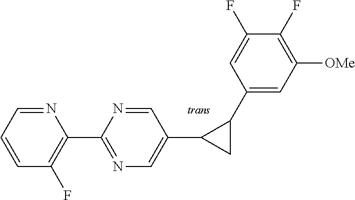 [SMILES: COc1cc(C2CC2c2cnc(-c3ncccc3F)nc2)cc(F)c1F] | trans-5-(2-(3,4-difluoro-5- methoxyphenyl)cyclopropyl)- 2-(3-fluoropyridin-2- yl)pyrimidine | 0.37 |
| 180 | 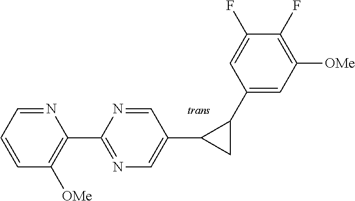 [SMILES: COc1cccnc1-c1ncc(C2CC2c2cc(F)c(F)c(OC)c2)cn1] | trans-5-(2-(3,4-difluoro-5- methoxyphenyl)cyclopropyl)- 2-(3-methoxypyridin-2- yl)pyrimidine | 1 |
| 181 | 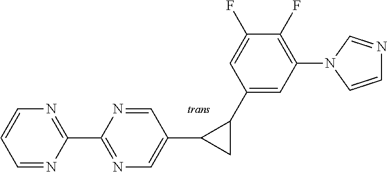 [SMILES: Fc1cc(C2CC2c2cnc(-c3ncccn3)nc2)cc(-n2ccnc2)c1F] | trans-5-(2-(3,4-difluoro-5- (1H-imidazol-1- yl)phenyl)cyclopropyl)-2,2'- bipyrimidine (single enantiomer I) | 0.39 |
| 182 | 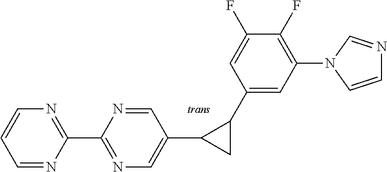 [SMILES: Fc1cc(C2CC2c2cnc(-c3ncccn3)nc2)cc(-n2ccnc2)c1F] | trans-5-(2-(3,4-difluoro-5- (1H-imidazol-1- yl)phenyl)cyclopropyl)-2,2'- bipyrimidine (single enantiomer II) | 0.064 |
| 183 | 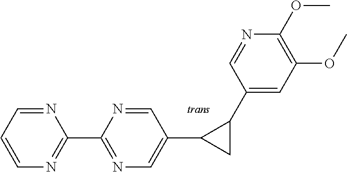 [SMILES: COc1cc(C2CC2c2cnc(-c3ncccn3)nc2)cnc1OC] | trans-5-(2-(5,6- dimethoxypyridin-3- yl)cyclopropyl)-2,2'- bipyrimidine (single enantiomer I) | 0.91 |
| 184 | 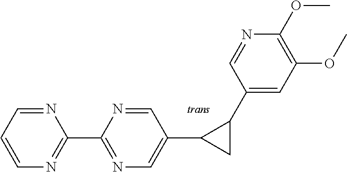 [SMILES: COc1cc(C2CC2c2cnc(-c3ncccn3)nc2)cnc1OC] | trans-5-(2-(5,6- dimethoxypyridin-3- yl)cyclopropyl)-2,2'- bipyrimidine (single enantiomer II) | 1.1 |
| 185 | 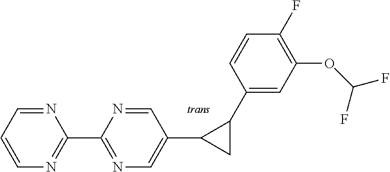 [SMILES: Fc1ccc(C2CC2c2cnc(-c3ncccn3)nc2)cc1OC(F)F] | trans-5-(2-(3- (difluoromethoxy)-4- fluorophenyl)cyclopropyl)- 2,2'-bipyrimidine (single enantiomer I) | 0.12 |
| 186 | 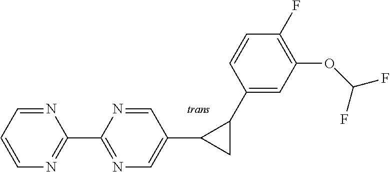 [SMILES: Fc1ccc(C2CC2c2cnc(-c3ncccn3)nc2)cc1OC(F)F] | trans-5-(2-(3- (difluoromethoxy)-4- fluorophenyl)cyclopropyl)- 2,2'-bipyrimidine (single enantiomer II) | 1.6 |
| 187 | 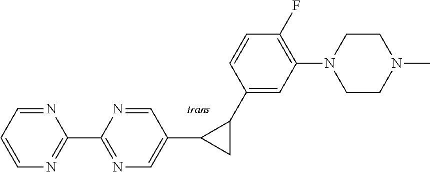 [SMILES: CN1CCN(c2cc(C3CC3c3cnc(-c4ncccn4)nc3)ccc2F)CC1] | trans-5-(2-(4-fluoro-3-(4- methylpiperazin-1- yl)phenyl)cyclopropyl)-2,2'- bipyrimidine (single enantiomer I) | 15 |
| 188 | 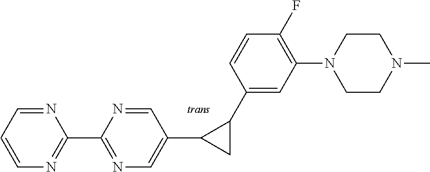 [SMILES: CN1CCN(c2cc(C3CC3c3cnc(-c4ncccn4)nc3)ccc2F)CC1] | trans-5-(2-(4-fluoro-3-(4- methylpiperazin-1- yl)phenyl)cyclopropyl)-2,2'- bipyrimidine (single enantiomer II) | 4 |
| 189 | 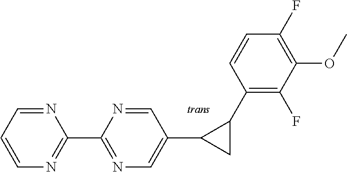 [SMILES: COc1c(F)ccc(C2CC2c2cnc(-c3ncccn3)nc2)c1F] | trans-5-(2-(2,4-difluoro-3- methoxyphenyl)cyclopropyl)- 2,2'-bipyrimidine (single enantiomer I) | 0.12 |
| 190 | 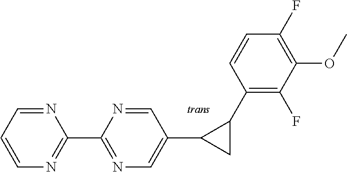 [SMILES: COc1c(F)ccc(C2CC2c2cnc(-c3ncccn3)nc2)c1F] | trans-5-(2-(2,4-difluoro-3- methoxyphenyl)cyclopropyl)- 2,2'-bipyrimidine (single enantiomer II) | 1.3 |
| 191 | 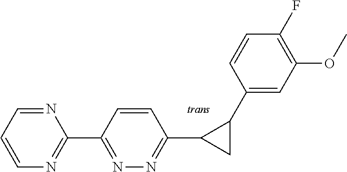 [SMILES: COc1cc(C2CC2c2ccc(-c3ncccn3)nn2)ccc1F] | trans-3-(2-(4-fluoro-3- methoxyphenyl)cyclopropyl)- 6-(pyrimidin-2- yl)pyridazine (single enantiomer I) | 2 |
| 192 | 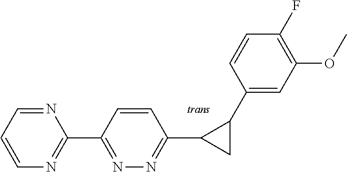 [SMILES: COc1cc(C2CC2c2ccc(-c3ncccn3)nn2)ccc1F] | trans-3-(2-(4-fluoro-3- methoxyphenyl)cyclopropyl)- 6-(pyrimidin-2- yl)pyridazine (single enantiomer II) | 49 |
| 193 | 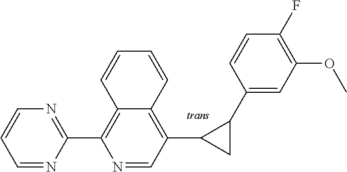 [SMILES: COc1cc(C2CC2c2cnc(-c3ncccn3)c3ccccc23)ccc1F] | trans-4-(2-(4-fluoro-3- methoxyphenyl)cyclopropyl)- 1-(pyrimidin-2- yl)isoquinoline (single enantiomer I) | 43 |
| 194 | 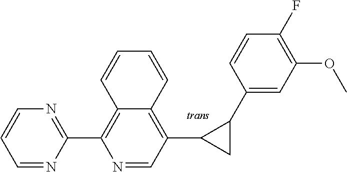 [SMILES: COc1cc(C2CC2c2cnc(-c3ncccn3)c3ccccc23)ccc1F] | trans-4-(2-(4-fluoro-3- methoxyphenyl)cyclopropyl)- 1-(pyrimidin-2- yl)isoquinoline (single enantiomer II) | 49 |
| 195 | 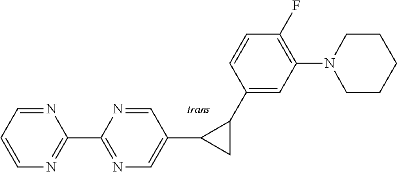 [SMILES: Fc1ccc(C2CC2c2cnc(-c3ncccn3)nc2)cc1N1CCCCC1] | trans-5-(2-(4-fluoro-3- (piperidin-1- yl)phenyl)cyclopropyl)-2,2'- bipyrimidine (single enantiomer I) | 3 |
| 196 | 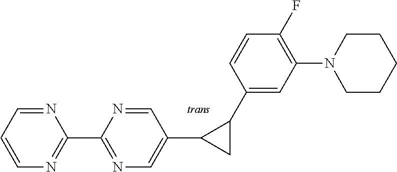 [SMILES: Fc1ccc(C2CC2c2cnc(-c3ncccn3)nc2)cc1N1CCCCC1] | trans-5-(2-(4-fluoro-3- (piperidin-1- yl)phenyl)cyclopropyl)-2,2'- bipyrimidine (single enantiomer II) | 0.21 |
| 197 | 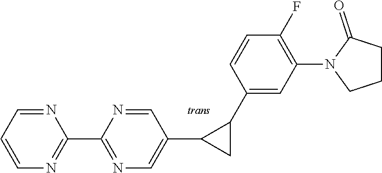 [SMILES: O=C1CCCN1c1cc(C2CC2c2cnc(-c3ncccn3)nc2)ccc1F] | trans-1-(5-(2-([2,2'- bipyrimidin]-5- yl)cyclopropyl)-2- fluorophenyl)pyrrolidin-2- one (single enantiomer I) | 3 |
| 198 | 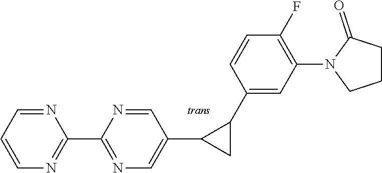 [SMILES: O=C1CCCN1c1cc(C2CC2c2cnc(-c3ncccn3)nc2)ccc1F] | trans-1-(5-(2-([2,2'- bipyrimidin]-5- yl)cyclopropyl)-2- fluorophenyl)pyrrolidin-2- one (single enantiomer II) | 0.43 |
| 199 | 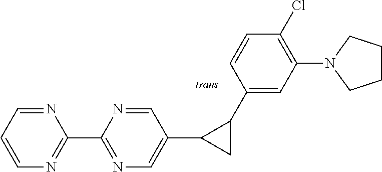 [SMILES: Clc1ccc(C2CC2c2cnc(-c3ncccn3)nc2)cc1N1CCCC1] | trans-5-(2-(4-chloro-3- (pyrrolidin-1- yl)phenyl)cyclopropyl)-2,2'- bipyrimidine (single enantiomer I) | 0.14 |
| 200 | 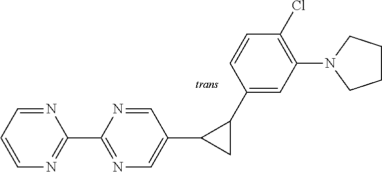 [SMILES: Clc1ccc(C2CC2c2cnc(-c3ncccn3)nc2)cc1N1CCCC1] | trans-5-(2-(4-chloro-3- (pyrrolidin-1- yl)phenyl)cyclopropyl)-2,2'- bipyrimidine (single enantiomer II) | 0.057 |
| 201 | 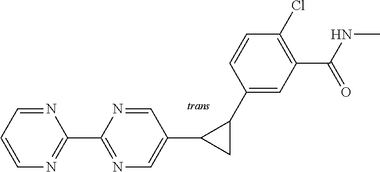 [SMILES: CNC(=O)c1cc(C2CC2c2cnc(-c3ncccn3)nc2)ccc1Cl] | trans-5-((2-([2,2'- bipyrimidin]-5- yl)cyclopropyl)-2-chloro-N- methylbenzamide (single enantiomer I) | 9 |
| 202 | 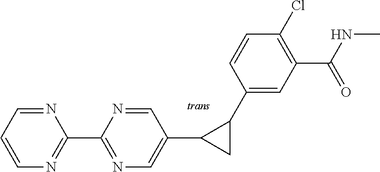 [SMILES: CNC(=O)c1cc(C2CC2c2cnc(-c3ncccn3)nc2)ccc1Cl] | trans-5-((2-([2,2'- bipyrimidin]-5- yl)cyclopropyl)-2-chloro-N- methylbenzamide (single enantiomer II) | 4 |
| 203 | 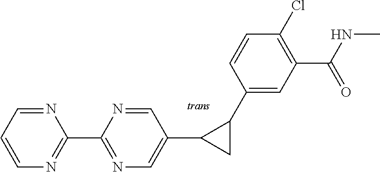 [SMILES: CNC(=O)c1cc(C2CC2c2cnc(-c3ncccn3)nc2)ccc1Cl] | trans-5-(2-([2,2'- bipyrimidin]-5- yl)cyclopropyl)-2-chloro- N,N-dimethylbenzamide (single enantiomer I) | 6 |
| 204 | 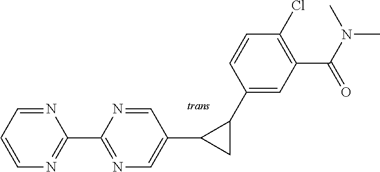 [SMILES: CN(C)C(=O)c1cc(C2CC2c2cnc(-c3ncccn3)nc2)ccc1Cl] | trans-5-(2-([2,2'- bipyrimidin]-5- yl)cyclopropyl)-2-chloro- N,N-dimethylbenzamide (single enantiomer II) | 4 |
| 205 | 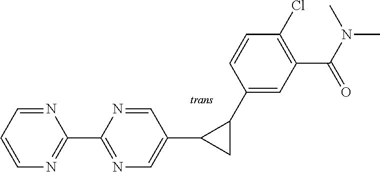 [SMILES: CN(C)C(=O)c1cc(C2CC2c2cnc(-c3ncccn3)nc2)ccc1Cl] | trans-N-(5-(2-([2,2'- bipyrimidin]-5- yl)cyclopropyl)-2- chlorophenyl)acetamide (single enantiomer I) | 0.51 |
| 206 | 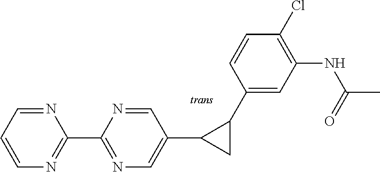 [SMILES: CC(=O)Nc1cc(C2CC2c2cnc(-c3ncccn3)nc2)ccc1Cl] | trans-N-(5-(2-([2,2'- bipyrimidin]-5- yl)cyclopropyl)-2- chlorophenyl)acetamide (single enantiomer II) | 0.82 |
| 207 | 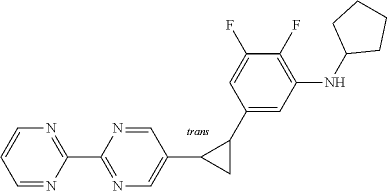 [SMILES: Fc1cc(C2CC2c2cnc(-c3ncccn3)nc2)cc(NC2CCCC2)c1F] | trans-5-(2-([2,2'- bipyrimidin]-5- yl)cyclopropyl)-N- cyclopentyl-2,3- difluoroaniline (single enantiomer I) | 0.75 |
| 208 | 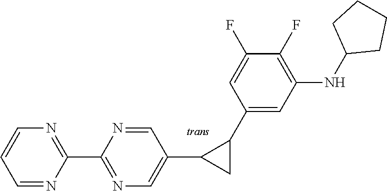 [SMILES: Fc1cc(C2CC2c2cnc(-c3ncccn3)nc2)cc(NC2CCCC2)c1F] | trans-5-(2-([2,2'- bipyrimidin]-5- yl)cyclopropyl)-N- cyclopentyl-2,3- difluoroaniline (single enantiomer II) | 0.11 |
| 209 | 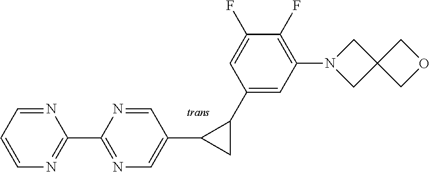 [SMILES: Fc1cc(C2CC2c2cnc(-c3ncccn3)nc2)cc(N2CC3(COC3)C2)c1F] | trans-6-( 5-(2-([2,2'- bipyrimidin]-5- yl)cyclopropyl)-2,3- difluorophenyl)-2-oxa-6- azaspiro[3.3]heptane | 0.12 |
| 210 | 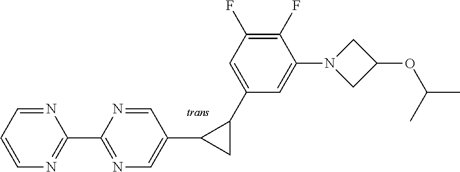 [SMILES: CC(C)OC1CN(c2cc(C3CC3c3cnc(-c4ncccn4)nc3)cc(F)c2F)C1] | trans-5-(2-(3,4-difluoro-5- (3-isopropoxyazetidin-1- yl)phenyl)cyclopropyl)-2,2'- bipyrimidine | 0.055 |
| 211 | 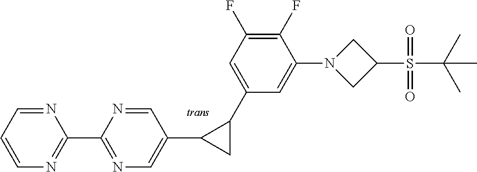 [SMILES: CC(C)(C)S(=O)(=O)C1CN(c2cc(C3CC3c3cnc(-c4ncccn4)nc3)cc(F)c2F)C1] | trans-5-(2-(3-(3-(tert- butylsulfonyl)azetidin-1-yl)- 4,5- difluorophenyl)cyclopropyl)- 2,2'-bipyrimidine | 0.11 |
| 212 | 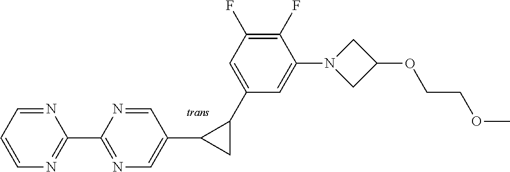 [SMILES: COCCOC1CN(c2cc(C3CC3c3cnc(-c4ncccn4)nc3)cc(F)c2F)C1] | trans-5-(2-(3,4-difluoro-5- (3-(2- methoxyethoxy)azetidin-1- yl)phenyl)cyclopropyl)-2,2'- bipyrimidine | 0.03 |
| 213 | 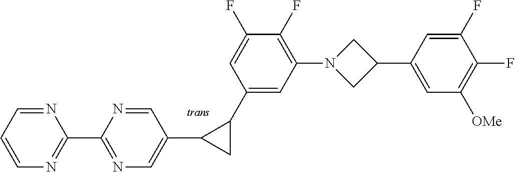 [SMILES: COc1cc(C2CN(c3cc(C4CC4c4cnc(-c5ncccn5)nc4)cc(F)c3F)C2)cc(F)c1F] | trans-5-(1-{3-(3-(3,4- difluoro-5- methoxyphenyl)azetidin-1- yl)-4,5- difluorophenyl)cyclopropyl)- 2,2,-bipyrimidine | 0.073 |
| 214 | 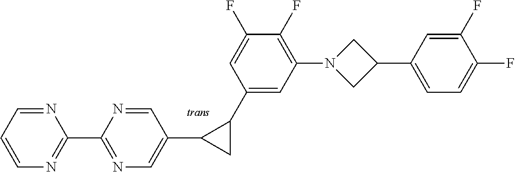 [SMILES: Fc1ccc(C2CN(c3cc(C4CC4c4cnc(-c5ncccn5)nc4)cc(F)c3F)C2)cc1F] | trans-5-(2-(3-(3-(3,4- difluorophenyl)azetidin-1- yl)-4,5- difluorophenyl)cyclopropyl)- 2,2'-bipyrimidine | 0.046 |
| 215 | 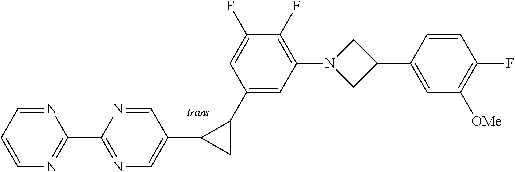 [SMILES: COc1cc(C2CN(c3cc(C4CC4c4cnc(-c5ncccn5)nc4)cc(F)c3F)C2)ccc1F] | trans-5-(2-(3,4-difluoro-5- (3-(4-fluoro-3- methoxyphenyl)azetidin-1- yl)phenyl)cyclopropyl)-2,2'- bipyrimidine | 0.07 |
| 216 | 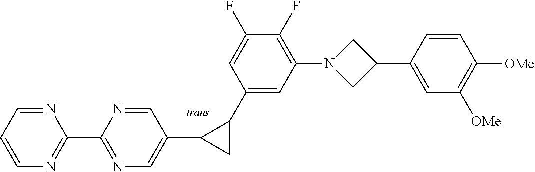 [SMILES: COc1ccc(C2CN(c3cc(C4CC4c4cnc(-c5ncccn5)nc4)cc(F)c3F)C2)cc1OC] | trans-5-(2-(3-(3-(3,4- dimethoxyphenyl)azetidin-1- yl)-4,5- difluorophenyl)cyclopropyl)- 2,2'-bipyrimidine | 0.025 |
| 217 | 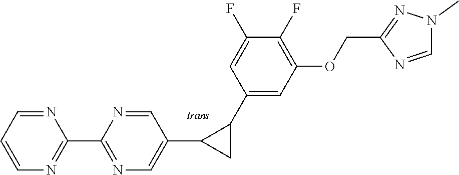 [SMILES: Cn1cnc(COc2cc(C3CC3c3cnc(-c4ncccn4)nc3)cc(F)c2F)n1] | trans-5-(2-(3,4-difluoro-5- ((1-methyl-1H-1,2,4-triazol- 3-yl)methoxy)phenyl) cyclopropyl)-2,2'-bipyrimidine | 0.20 |
| 218 | 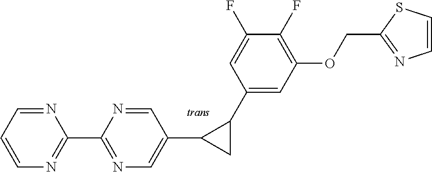 [SMILES: Fc1cc(C2CC2c2cnc(-c3ncccn3)nc2)cc(OCc2nccs2)c1F] | trans-2-((5-(2-([2,2'- bipyrimidin]-5- yl)cyclopropyl)-2,3- difluorophenoxy)methyl) thiazole | 0.057 |
| 219 | 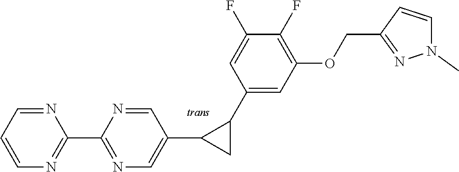 [SMILES: Cn1ccc(COc2cc(C3CC3c3cnc(-c4ncccn4)nc3)cc(F)c2F)n1] | trans-5-(2-(3,4-difluoro-5- ((1-methyl-1H-pyrazol-3- yl)methoxy)phenyl) cyclopropyl)-2,2'-bipyrimidine | 0.096 |
| 220 | 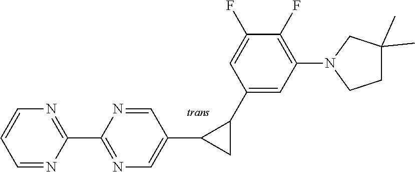 [SMILES: CC1(C)CCN(c2cc(C3CC3c3cnc(-c4ncccn4)nc3)cc(F)c2F)C1] | trans-5-(2-(3-(3,3- dimethylpyrrolidin-1-yl)-4,5- difluorophenyl)cyclopropyl)- 2,2'-bipyrimidine (single enantiomer I) | 0.03 |
| 221 | 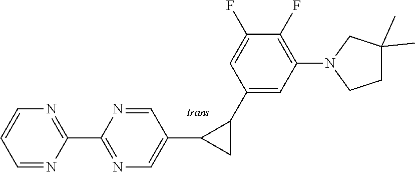 [SMILES: CC1(C)CCN(c2cc(C3CC3c3cnc(-c4ncccn4)nc3)cc(F)c2F)C1] | trans-5-(2-(3-(3,3- dimethylpyrrolidin-1-yl)-4,5- difluorophenyl)cyclopropyl)- 2,2'-bipyrimidine (single enantiomer II) | 0.32 |
| 222 | 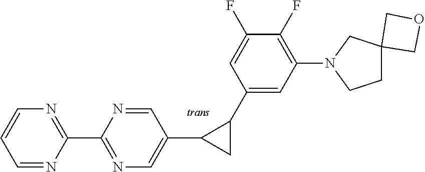 [SMILES: Fc1cc(C2CC2c2cnc(-c3ncccn3)nc2)cc(N2CCC3(COC3)C2)c1F] | trans-6-(5-(2-([2,2'- bipyrimidin]-5- yl)cyclopropyl)-2,3- difluorophenyl)-2-oxa-6- azaspiro[3.4]octane (single enantiomer I) | 0.19 |
| 223 | 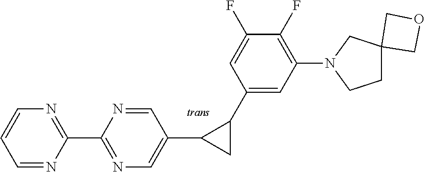 [SMILES: Fc1cc(C2CC2c2cnc(-c3ncccn3)nc2)cc(N2CCC3(COC3)C2)c1F] | trans-6-(5-(2-([2,2'- bipyrimidin]-5- yl)cyclopropyl)-2,3- difluorophenyl)-2-oxa-6- azaspiro[3.4]octane (single enantiomer II) | 0.01 |
| 224 | 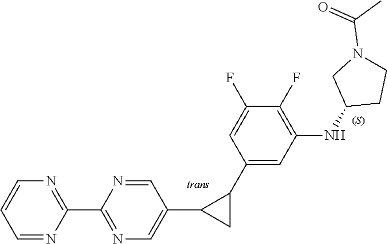 | trans-1-((3S)-3-((5-(2-([2,2'- bipyrimidin]-5- yl)cyclopropyl)-2,3- difluorophenyl)amino) pyrrolidin-1-yl)ethenone (single diastereomer I) | 5 |
| 225 | 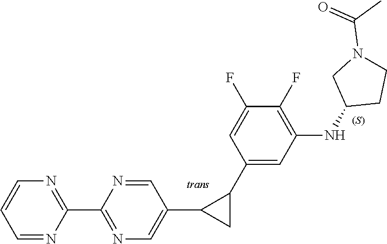 | trans-1-((3S)-3-((5-(2-([2,2'- bipyrimidin]-5- yl)cyclopropyl)-2,3- difluorophenyl)amino) pyrrolidin-1-yl)ethenone (single diastereomer II) | 0.35 |
| 226 | 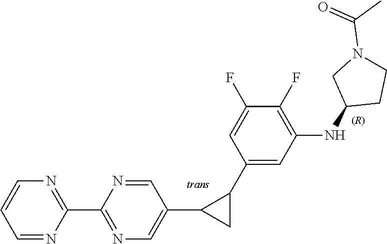 | trans-1-((3R)-3-((5-(2-([2,2'- bipyrimidin]-5- yl)cyclopropyl)-2,3- difluorophenyl)amino) pyrrolidin-1-yl)ethenone (single diastereomer I) | 1 |
| 227 | 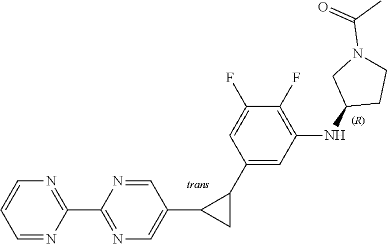 | trans-1-((3R)-3-((5-(2-([2,2'- bipyrimidin]-5- yl)cyclopropyl)-2,3- difluorophenyl)amino) pyrrolidin-1-yl)ethenone (single diastereomer II) | 0.27 |
| 228 | 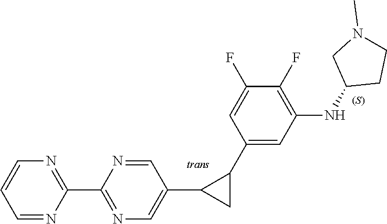 | trans-(3S)-N-(5-(2-([2,2'- bipyrimidin]-5- yl)cyclopropyl)-2,3- difluorophenyl)-1- methylpyrrolidin-3-amine (single diastereomer I) | 15 |
| 229 | 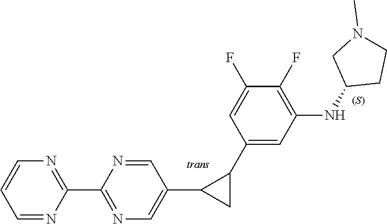 | trans-(3S)-N-(5-(2-([2,2'- bipyrimidin]-5- yl)cyclopropyl)-2,3- difluorophenyl)-1- methylpyrrolidin-3-amine (single diastereomer II) | 3 |
| 230 | 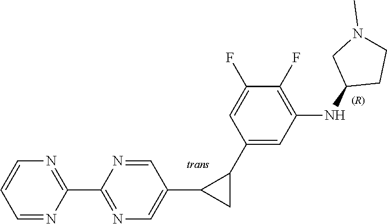 | trans-(3R)-N-(5-(2-([2,2'- bipyrimidin]-5- yl)cyclopropyl)-2,3- difluorophenyl)-1- methylpyrrolidin-3-amine (single diastereomer I) | 7 |
| 231 | 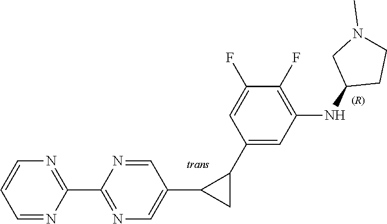 | trans-(3R)-N-(5-(2-([2,2'- bipyrimidin]-5- yl)cyclopropyl)-2,3- difluorophenyl)-1- methylpyrrolidin-3-amine (single diastereomer II) | 0.75 |
| 232 | 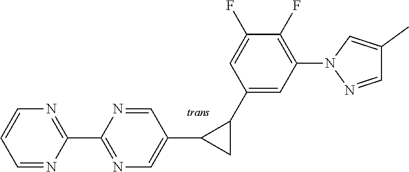 [SMILES: Cc1cnn(-c2cc(C3CC3c3cnc(-c4ncccn4)nc3)cc(F)c2F)c1] | trans-5-(2-(3,4-difluoro-5- (4-methyl-1H-pyrazol-1- yl)phenyl)cyclopropyl)-2,2'- bipyrimidine | 0.028 |
| 233 | 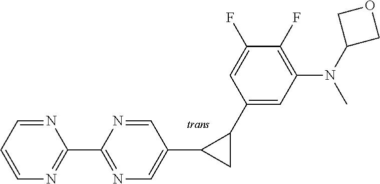 [SMILES: CN(c1cc(C2CC2c2cnc(-c3ncccn3)nc2)cc(F)c1F)C1COC1] | trans-N-(5-(2-([2,2'- bipyrimidin]-5- yl)cyclopropyl)-2,3- difluorophenyl)-N- methyloxetan-3-amine | 0.084 |
| 234 | 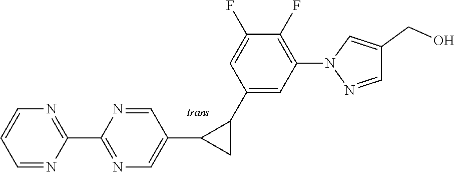 [SMILES: OCc1cnn(-c2cc(C3CC3c3cnc(-c4ncccn4)nc3)cc(F)c2F)c1] | trans-(1-(5-(2-([2,2'- bipyrimidin]-5- yl)cyclopropyl)-2,3- difluorophenyl)-1H-pyrazol- 4-yl)methanol | 0.14 |
| 235 | 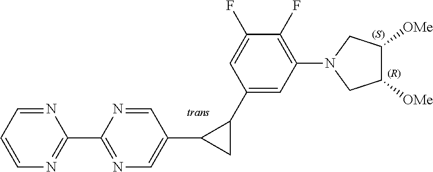 | trans-5-(2-(3-((3R,4S)-3,4- dimethoxypyrrolidin-1-yl)- 4,5- difluorophenyl)cyclopropyl)- 2,2'-bipyrimidine | 0.039 |
| 236 | 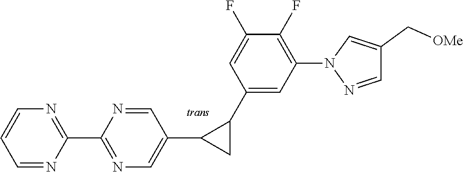 [SMILES: COCc1cnn(-c2cc(C3CC3c3cnc(-c4ncccn4)nc3)cc(F)c2F)c1] | trans-5-(2-(3,4-difluoro-5- (4-(methoxymethyl)-1H- pyrazol-1- yl)phenyl)cyclopropyl)-2,2'- bipyrimidine | 0.078 |
| 237 | 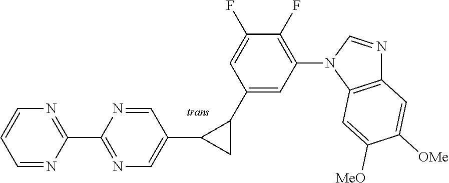 [SMILES: COc1cc2ncn(-c3cc(C4CC4c4cnc(-c5ncccn5)nc4)cc(F)c3F)c2cc1OC] | trans-1-(5-(2-([2,2- bipyrimidin]-5- yl)cyclopropyl)-2,3- difluorophenyl)-5,6- dimethoxy-1H- benzo[d]imidazole | 0.2 |
| 238 | 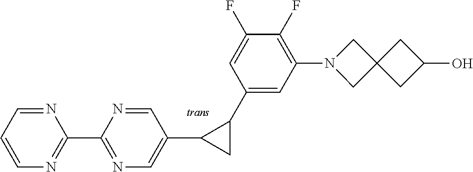 [SMILES: OC1CC2(C1)CN(c1cc(C3CC3c3cnc(-c4ncccn4)nc3)cc(F)c1F)C2] | trans-2-(5-(2-([2,2'- bipyrimidin]-5- yl)cyclopropyl)-2,3- difluorophenyl)-2- azaspiro[3,3]heptan-6-ol | 0.053 |
| 239 | 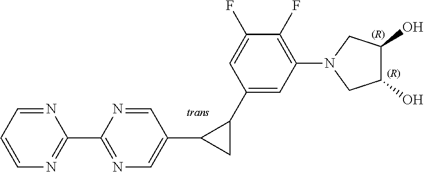 | trans-(3R,4R)-1-(5-(2-([2,2'- bipyrimidin]-5- yl)cyclopropyl)-2,3- difluorophenyl)pyrrolidine- 3,4-diol | 0.11 |
| 240 | 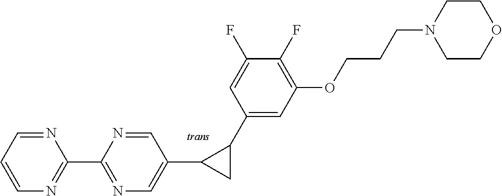 [SMILES: Fc1cc(C2CC2c2cnc(-c3ncccn3)nc2)cc(OCCCN2CCOCC2)c1F] | trans-4-(3-(5-(2-([2,2'- bipyrimidin]-5- yl)cyclopropyl)-2,3- difluorophenoxy)propyl) morpholine | 0.21 |
| 241 | 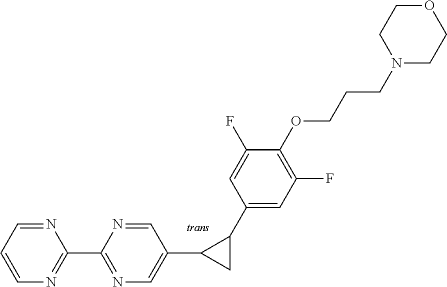 [SMILES: Fc1cc(C2CC2c2cnc(-c3ncccn3)nc2)cc(F)c1OCCCN1CCOCC1] | trans-4-(3-(4-(2-([2,2'- bipyrimidin]-5- yl)cyclopropyl)-2,6- difluorophenoxy)propyl) morpholine | 0.83 |
| 242 | 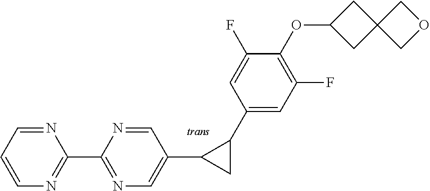 [SMILES: Fc1cc(C2CC2c2cnc(-c3ncccn3)nc2)cc(F)c1OC1CC2(COC2)C1] | trans-5-(2-(4-((2- oxaspiro[3.3]heptan-6- yl)oxy)-3,5- difluorophenyl)cyclopropyl)- 2,2'-bipyrimidine | 0.39 |
| 243 | 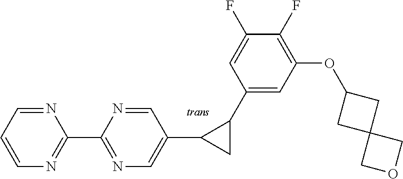 [SMILES: Fc1cc(C2CC2c2cnc(-c3ncccn3)nc2)cc(OC2CC3(COC3)C2)c1F] | trans-5-(2-(3-((2- oxaspiro[3.3]heptan-6- yl)oxy)-4,5- difluorophenyl)cyclopropyl)- 2,2'-bipyrimidine | 0.11 |
| 244 | 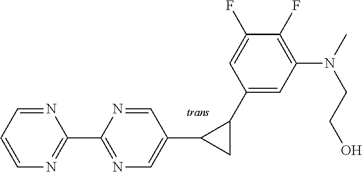 [SMILES: CN(CCO)c1cc(C2CC2c2cnc(-c3ncccn3)nc2)cc(F)c1F] | trans-2-((5-(2-([2,2'- bipyrimidin]-5- yl)cyclopropyl)-2,3- difluorophenyl)(methyl) amino)ethan-1-ol | 0.092 |
| 245 | 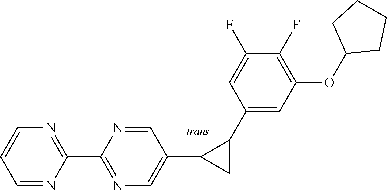 [SMILES: Fc1cc(C2CC2c2cnc(-c3ncccn3)nc2)cc(OC2CCCC2)c1F] | trans-5-(2-(3- (cyclopentyloxy)-4,5- difluorophenyl)cyclopropyl)- 2,2'-bipyrimidine (single enantiomer I) | 5 |
| 246 | 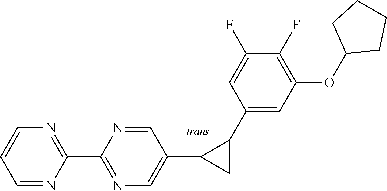 [SMILES: Fc1cc(C2CC2c2cnc(-c3ncccn3)nc2)cc(OC2CCCC2)c1F] | trans-5-(2-(3- (cyclopentyloxy)-4,5- difluorophenyl)cyclopropyl)- 2,2'-bipyrimidine (single enantiomer II) | 0.15 |
| 247 | 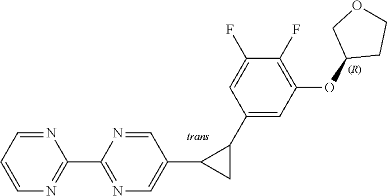 | trans-5-(2-(3,4-difluoro-5- (((R)-tetrahydrofuran-3- yl)oxy)phenyl)cyclopropyl)- 2,2'-bipyrimidine (single diastereomer I) | 0.076 |
| 248 | 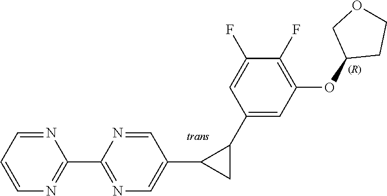 | trans-5-(2-(3,4-difluoro-5- (((R)-tetrahydrofuran-3- yl)oxy)phenyl)cyclopropyl)- 2,2'-bipyrimidine (single diastereomer II) | 1 |
| 249 | 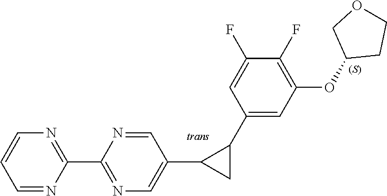 | trans-5-(2-(3,4-difluoro-5- (((S)-tetrahydrofuran-3- yl)oxy)phenyl)cyclopropyl)- 2,2'-bipyrimidine (single diastereomer I) | 0.8 |
| 250 | 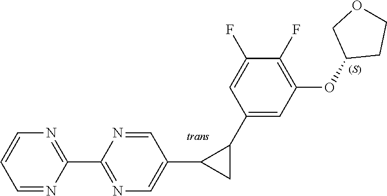 | trans-5-(2-(3,4-difluoro-5- (((S)-tetrahydrofuran-3- yl)oxy)phenyl)cyclopropyl)- 2,2'-bipyrimidine (single diastereomer II) | 0.076 |
| 251 | 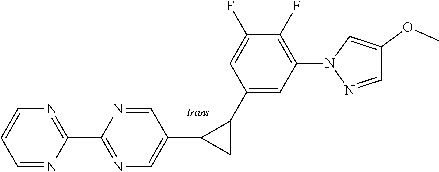 [SMILES: COc1cnn(-c2cc(C3CC3c3cnc(-c4ncccn4)nc3)cc(F)c2F)c1] | trans-5-(2-(3,4-difluoro-5- (4-methoxy-1H-pyrazol-1- yl)phenyl)cyclopropyl)-2,2'- bipyrimidine (single enantiomer I) | 0.041 |
| 252 | 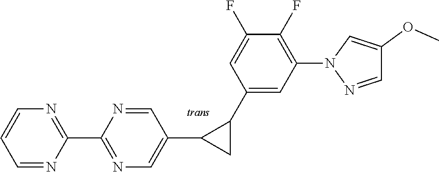 [SMILES: COc1cnn(-c2cc(C3CC3c3cnc(-c4ncccn4)nc3)cc(F)c2F)c1] | trans-5-(2-(3,4-difluoro-5- (4-methoxy-1H-pyrazol-1- yl)phenyl)cyclopropyl)-2,2'- bipyrimidine (single enantiomer II) | 0.014 |
| 253 | 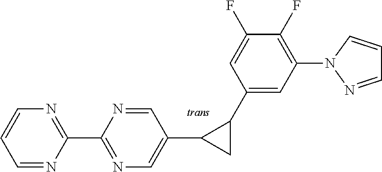 [SMILES: Fc1cc(C2CC2c2cnc(-c3ncccn3)nc2)cc(-n2cccn2)c1F] | trans-5-(2-(3,4-difluoro-5- (1H-pyrazol-1- yl)phenyl)cyclopropyl)-2,2'- bipyrimidine (single enantiomer I) | 0.029 |
| 254 | 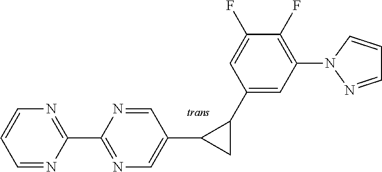 [SMILES: Fc1cc(C2CC2c2cnc(-c3ncccn3)nc2)cc(-n2cccn2)c1F] | trans-5-(2-(3,4-difluoro-5- (1H-pyrazol-1- yl)phenyl)cyclopropyl)-2,2'- bipyrimidine (single enantiomer II) | 0.047 |
| 255 | 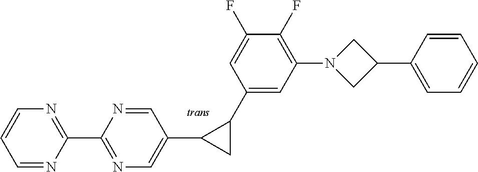 [SMILES: Fc1cc(C2CC2c2cnc(-c3ncccn3)nc2)cc(N2CC(c3ccccc3)C2)c1F] | trans-5-(2-(3,4-difluoro-5- (3-phenylazetidin-1- yl)phenyl)cyclopropyl)-2,2'- bipyrimidine (single enantiomer I) | 0.78 |
| 256 | 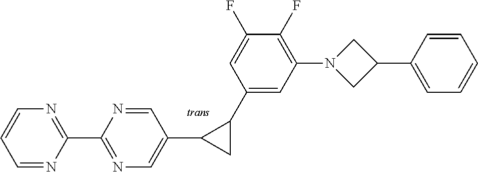 [SMILES: Fc1cc(C2CC2c2cnc(-c3ncccn3)nc2)cc(N2CC(c3ccccc3)C2)c1F] | trans-5-(2-(3,4-difluoro-5- (3-phenylazetidin-1- yl)phenyl)cyclopropyl)-2,2'- bipyrimidine (single enantiomer II) | 0.028 |
| 257 | 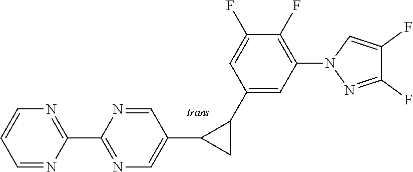 [SMILES: Fc1cn(-c2cc(C3CC3c3cnc(-c4ncccn4)nc3)cc(F)c2F)nc1F] | trans-5-(2-(3-(3,4-difluoro- 1H-pyrrol-1-yl)-4,5- difluorophenyl)cyclopropyl)- 2,2'-bipyrimidine (single enantiomer I) | 0.14 |
| 258 | 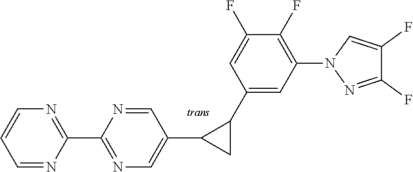 [SMILES: Fc1cn(-c2cc(C3CC3c3cnc(-c4ncccn4)nc3)cc(F)c2F)nc1F] | trans-5-(2-(3-(3,4-difluoro- 1H-pyrrol-1-yl)-4,5- difluorophenyl)cyclopropyl)- 2,2'-bipyrimidine (single enantiomer II) | 0.014 |
| 259 | 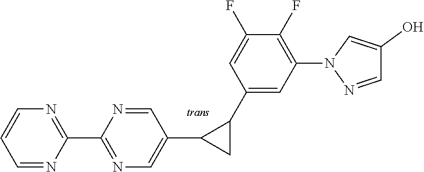 [SMILES: Oc1cnn(-c2cc(C3CC3c3cnc(-c4ncccn4)nc3)cc(F)c2F)c1] | trans-1-(5-(2-([2,2'- bipyrimidin]-5- yl)cyclopropyl)-2,3- difluorophenyl)-1H-pyrazol- 4-ol (single enantiomer I) | 2 |
| 260 | 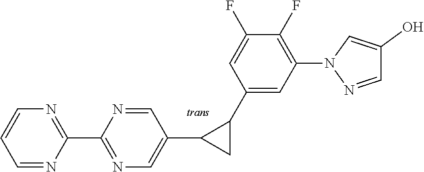 [SMILES: Oc1cnn(-c2cc(C3CC3c3cnc(-c4ncccn4)nc3)cc(F)c2F)c1] | trans-1-(5-(2-([2,2'- bipyrimidin]-5- yl)cyclopropyl)-2,3- difluorophenyl)-1H-pyrazol- 4-ol (single enantiomer II) | 0.67 |
| 261 | 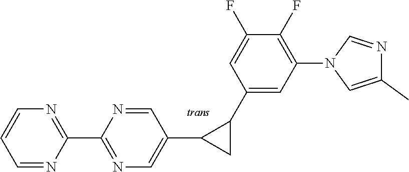 [SMILES: Cc1cn(-c2cc(C3CC3c3cnc(-c4ncccn4)nc3)cc(F)c2F)cn1] | trans-5-(2-(3,4-difluoro-5- (4-methyl-1H-imidazol-1- yl)phenyl)cyclopropyl)-2,2'- bipyrimidine (single enantiomer I) | 0.67 |
| 262 | 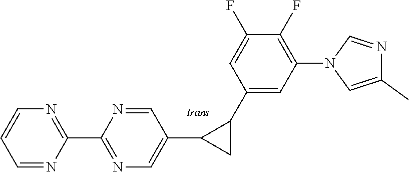 [SMILES: Cc1cn(-c2cc(C3CC3c3cnc(-c4ncccn4)nc3)cc(F)c2F)cn1] | trans-5-(2-(3,4-difluoro-5- (4-methyl-1H-imidazol-1- yl)phenyl)cyclopropyl)-2,2'- bipyrimidine (single enantiomer II) | 0.047 |
| 263 | 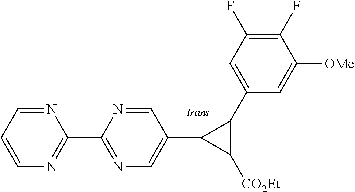 [SMILES: CCOC(=O)C1C(c2cnc(-c3ncccn3)nc2)C1c1cc(F)c(F)c(OC)c1] | trans-ethyl 2-([2,2'- bipyrimidin]-5-yl)-3-(3,4- difluoro-5- methoxyphenyl)cyclopropane- 1-carboxylate | 12 |
| 264 | 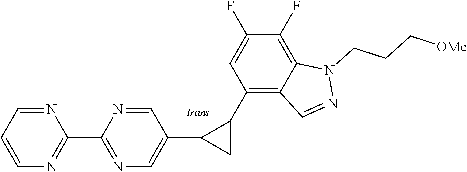 [SMILES: COCCCn1ncc2c(C3CC3c3cnc(-c4ncccn4)nc3)cc(F)c(F)c21] | trans-4-(2-([2,2'- bipyrimidin]-5- yl)cyclopropyl)-6,7-difluoro- 1-(3-methoxypropyl)-1H- indazole | 0.004 |
| 265 | 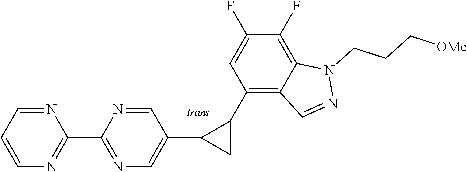 [SMILES: COCCCn1ncc2c(C3CC3c3cnc(-c4ncccn4)nc3)cc(F)c(F)c21] | trans-4-(2-([2,2'- bipyrimidin]-5- yl)cyclopropyl)-6,7-difluoro- 1-(3-methoxypropyl)-1H- indazole (single enantiomer I) | 0.061 |
| 266 | 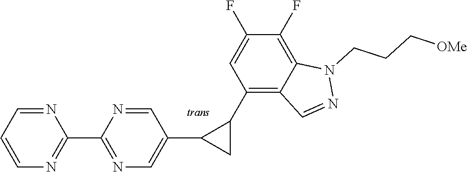 [SMILES: COCCCn1ncc2c(C3CC3c3cnc(-c4ncccn4)nc3)cc(F)c(F)c21] | trans-4-(2-([2,2'- bipyrimidin]-5- yl)cyclopropyl)-6,7-difluoro- 1-(3-methoxypropyl)-1H- indazole (single enantiomer II) | 0.002 |
| 267 | 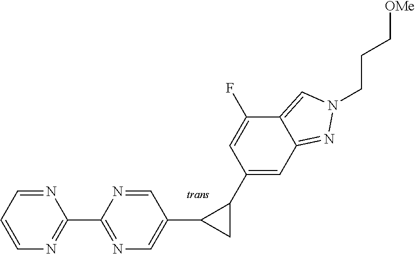 [SMILES: COCCCn1cc2c(F)cc(C3CC3c3cnc(-c4ncccn4)nc3)cc2n1] | trans-6-(2-([2,2'- bipyrimidin]-5- yl)cyclopropyl)-4-fluoro-2- (3-methoxypropyl)-2H- indazole | 0.013 |
| 268 | 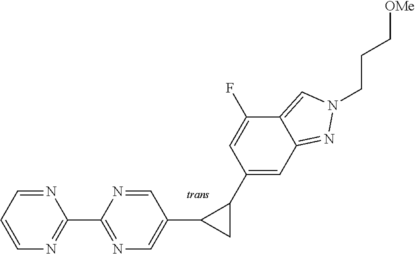 [SMILES: COCCCn1cc2c(F)cc(C3CC3c3cnc(-c4ncccn4)nc3)cc2n1] | trans-6-(2-([2,2'- bipyrimidin]-5- yl)cyclopropyl)-4-fluoro-2- (3-methoxypropyl)-2H- indazole (single enantiomer I) | 0.01 |
| 269 | 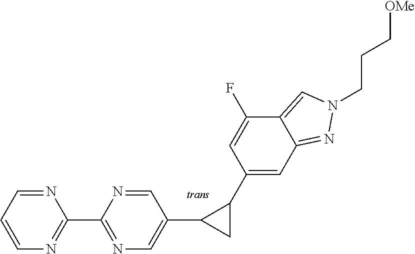 [SMILES: COCCCn1cc2c(F)cc(C3CC3c3cnc(-c4ncccn4)nc3)cc2n1] | trans-6-(2-([2,2'- bipyrimidin]-5- yl)cyclopropyl)-4-fluoro-2- (3-methoxypropyl)-2H- indazole (single enantiomer II) | 0.046 |
| 270 | 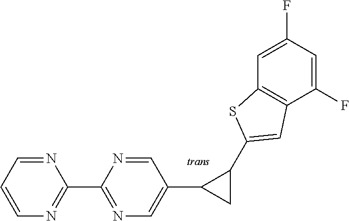 [SMILES: Fc1cc(F)c2cc(C3CC3c3cnc(-c4ncccn4)nc3)sc2c1] | trans-2-(2-([2,2'- bipyrimidin]-5- yl)cyclopropyl)-4,6- difluorobenzo[d]thiazole | 0.5 |
| 271 | 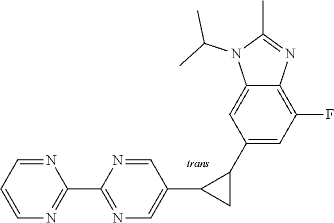 [SMILES: Cc1nc2c(F)cc(C3CC3c3cnc(-c4ncccn4)nc3)cc2n1C(C)C] | trans-6-(2-([2,2'- bipyrimidin]-5- yl)cyclopropyl)-4-fluoro-1- isopropyl-2-methyl-1H- benzo[d]imidazole | 0.25 |
| 272 | 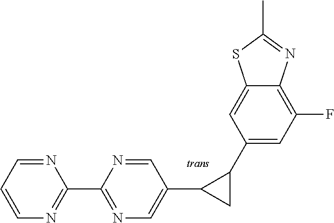 [SMILES: Cc1nc2c(F)cc(C3CC3c3cnc(-c4ncccn4)nc3)cc2s1] | trans-6-(2-([2,2'- bipyrimidin]-5- yl)cyclopropyl)-4-fluoro-2- methylbenzo[d]thiazole | 0.073 |
| 273 | 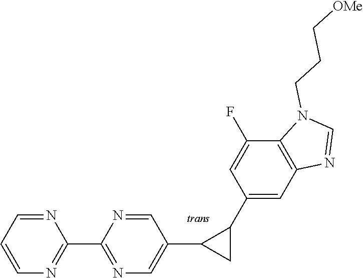 [SMILES: COCCCn1cnc2cc(C3CC3c3cnc(-c4ncccn4)nc3)cc(F)c21] | trans-5-(2-([2,2'- bipyrimidin]-5- yl)cyclopropyl)-7-fluoro-1- (3-methoxy propyl)-1H- benzo[d]iniidazole | 0.043 |
| 274 | 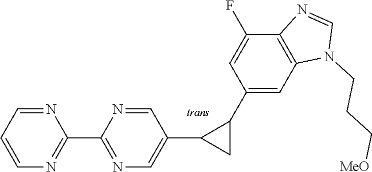 [SMILES: COCCCn1cnc2c(F)cc(C3CC3c3cnc(-c4ncccn4)nc3)cc21] | trans-6-(2-([2,2'- bipyrimidin]-5- yl)cyclopropyl)-4-fluoro-1- (3-methoxypropyl)-1H- benzo[d]imidazole | 0.064 |
| 275 | 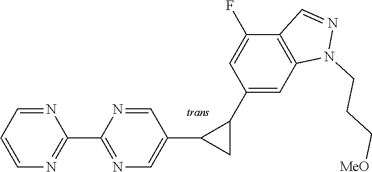 [SMILES: COCCCn1ncc2c(F)cc(C3CC3c3cnc(-c4ncccn4)nc3)cc21] | trans-6-(2-([2,2'- bipyrimidin]-5- yl)cyclopropyl)-4-fluoro-1- (3-methoxypropyl)-1H- indazole | 0.075 |
| 276 | 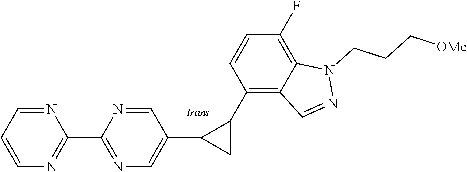 [SMILES: COCCCn1ncc2c(C3CC3c3cnc(-c4ncccn4)nc3)ccc(F)c21] | trams-4-(2-([2,2'- bipyrimidin]-5- yl)cyclopropyl)-7-fluoro-1- (3-methoxypropyl)-1H- indazole | 0.01 |
| 277 | 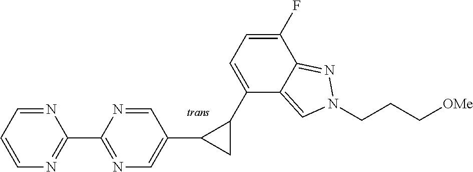 [SMILES: COCCCn1cc2c(C3CC3c3cnc(-c4ncccn4)nc3)ccc(F)c2n1] | trans-4-(2-([2,2'- bipyrimidin]-5- yl)cyclopropyl)-7-fluoro-2- (3-methoxypropyl)-2H- indazole | 0.03 |
| 278 | 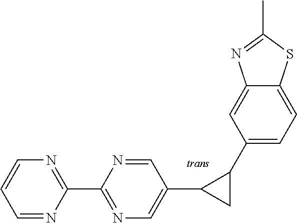 [SMILES: Cc1nc2cc(C3CC3c3cnc(-c4ncccn4)nc3)ccc2s1] | trans-5-(2-([2,2'- bipyrimidin]-5- yl)cyclopropyl)-2- methylbenzo[d]thiazole | 0.056 |

### Enumerated Embodiments

The following exemplary embodiments are provided, the numbering of which is not to be construed as designating levels of importance:

Embodiment 1 provides a compound of formula (I) or (II) or (III), or a salt, solvate, geometric isomer, stereoisomer, tautomer, and any mixtures thereof:

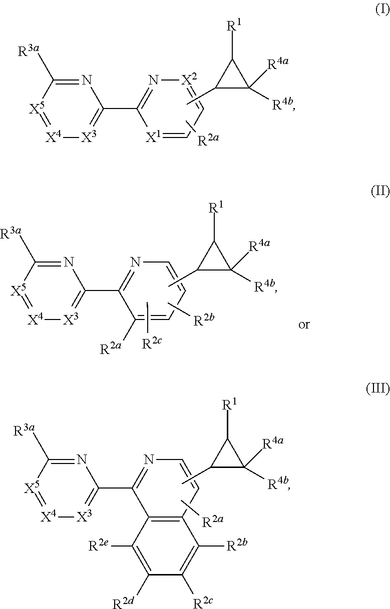

wherein:

one of the following applies: (i) X¹ is N, X² is CR^{2b}, or (ii) X¹ is CR^{2c}, X² is N;

one of the following applies: (i) X³ is N, X⁴ is CR^{3c}, X⁵ is CR^{3d}; or (ii) X³ is CR^{3b}, X⁴ is N, X⁵ is CR^{3d}; or (iii) X³ is CR^{3b}, X⁴ is CR^{3c}, X⁵ is N;

R¹ is selected from the group consisting of optionally substituted phenyl, optionally substituted naphthyl, optionally substituted pyridinyl, optionally substituted pyrimidinyl, optionally substituted benzo[d]thiazolyl; optionally substituted benzoimidazolyl, optionally substituted imidazo[1,2-a]pyridinyl, optionally substituted quinolinyl, optionally substituted isoquinolinyl, optionally substituted 1H-indazolyl, and optionally substituted 2H-indazolyl;

each occurrence of R^{2a}, R^{2b}, R^{2c}, R^{2d}, and R^{2e} is independently selected from the group consisting of H, halogen, cyano, nitro, optionally substituted C₁-C₆ alkyl, optionally substituted C₁-C₆ haloalkyl, optionally substituted C₃-C₈ cycloalkyl, optionally substituted C₁-C₆ haloalkoxy, optionally substituted C₁-C₆ hydroxyalkyl, —OR′, —SR′, —S(═O)R′, —S(O)₂R′, —N(R′)(R′), —N(R′)C(═O)(R′), —C(═O)N(R′)(R′), optionally substituted phenyl, optionally substituted heterocyclyl, and optionally substituted heteroaryl, wherein each occurrence of R′ is independently selected from the group consisting of H, optionally substituted C₁-C₆ alkyl, and optionally substituted C₃-C₈ cycloalkyl;

each occurrence of R^{3a}, R^{3b}, R^{3c}, and R^{3d} is independently selected from the group consisting of H, halogen, cyano, nitro, optionally substituted C₁-C₆ alkyl, optionally substituted C₁-C₆ haloalkyl, optionally substituted C₃-C₈ cycloalkyl, optionally substituted C₁-C₆ haloalkoxy, optionally substituted C₁-C₆ hydroxyalkyl, —OR″, —SR″, —S(═O)R″, —S(O)₂R″, —N(R″)(R″), optionally substituted phenyl, optionally substituted heterocyclyl, and optionally substituted heteroaryl, wherein each occurrence of R″ is independently selected from the group consisting of H, optionally substituted C₁-C₆ alkyl, and optionally substituted C₃-C₈ cycloalkyl;

R^{4a} is selected from the group consisting of R^{4c}, F, Cl, Br, I, —OR^{4c}, and —C(═O)OR^{4c}, wherein each occurrence of R^{4c} is independently H, optionally substituted C₁-C₆ alkyl, or optionally substituted C₃-C₈ cycloalkyl, and

R^{4b} is selected from the group consisting of R^{4d}, F, Cl, Br, I, —OR^{4d}, and —C(═O)OR^{4d}, wherein each occurrence of R^{4d} is independently H, optionally substituted C₁-C₆ alkyl, or optionally substituted C₃-C₈ cycloalkyl.

Embodiment 2 provides the compound of Embodiment 1, wherein in (I) X¹ is N and X² is CR^{2b}.

Embodiment 3 provides the compound of any of Embodiments 1-2, wherein R¹ is substituted with at least one selected from the group consisting of H, F, Cl, Br, I, optionally substituted C₁-C₆ alkyl, optionally substituted C₁-C₆ haloalkyl, optionally substituted C₁-C₆ alkoxy, optionally substituted C₁-C₆ haloalkoxy, optionally substituted phenyl, —NR″′Ir, —C(═O)NR″′Ir, —NHC(═O)R″′, and optionally substituted heterocyclyl; wherein each occurrence of R″′ is independently selected from the group consisting of H, optionally substituted C₁-C₆ alkyl, optionally substituted C₃-C₈ cycloalkyl, and optionally substituted heterocyclyl.

Embodiment 4 provides the compound of any of Embodiments 1-3, wherein R¹ is substituted with at least one selected from the group consisting of: H; F; Cl; Br; I; C₁-C₆ alkyl; C₁-C₆ alkyl substituted with at least one of F, Cl, Br, I, OH, CN, C₁-C₆ alkyl, C₃-C₈ cycloalkyl, C₁-C₆ alkoxy, and C₃-C₈ cycloalkoxy; C₁-C₆ alkoxy; C₁-C₆ alkoxy substituted with at least one of F, Cl, Br, I, OH, CN, C₁-C₆ alkyl, C₃-C₈ cycloalkyl, C₁-C₆ alkoxy, and C₃-C₈ cycloalkoxy; C₃-C₈ cycloalkyl; C₃-C₈ cycloalkyl substituted with at least one of F, Cl, Br, I, OH, CN, C₁-C₆ alkyl, C₃-C₈ cycloalkyl, C₁-C₆ alkoxy, and C₃-C₈ cycloalkoxy; C₃-C₈ cycloalkoxy; C₃-C₈ cycloalkoxy substituted with at least one of F, Cl, Br, I, OH, CN, C₁-C₆ alkyl, C₃-C₈ cycloalkyl, C₁-C₆ alkoxy, and C₃-C₈ cycloalkoxy; —NH₂; —NH(C₁-C₆ alkyl); —N(C₁-C₆ alkyl)(C₁-C₆ alkyl); —C(═O)NH(C₁-C₆ alkyl); —NHC(═O)H; —NHC(═O)(C₁-C₆ alkyl); optionally substituted phenyl; optionally substituted spiroheterocyclyl; optionally substituted pyrrolidinonyl; optionally substituted azetidinyl; optionally substituted pyrrolidinyl; optionally substituted pyrrolidinonyl; optionally substituted piperidinyl; optionally substituted piperazinyl; optionally substituted morpholinyl; optionally substituted pyrrolyl; optionally substituted pyrazolyl; optionally substituted imidazolyl; optionally substituted 1H-benzo[d]imidazyl; optionally substituted indazolyl; optionally substituted benzo[d]thiazolyl; and optionally substituted 1H-benzo[d]imidazolyl.

Embodiment 5 provides the compound of any of Embodiments 1-4, wherein R¹ is substituted with at least one selected from the group consisting of: H; F, Cl, Br, I, methyl, difluoromethyl, trifluoromethyl, ethyl, propyl, isopropyl, phenyl, methoxy, ethoxy, propoxy, isopropoxy, 2-methoxyethoxy, 3-methoxypropoxy, cyclopropylmethoxy, 2,2-difluoroethoxy, difluoromethoxy, trifluoromethoxy, (1-methyl-1H-1,2,4-triazol-3-yl)methoxy, (thiazol-2-yl)methoxy, (1-methyl-1H-pyrazol-3-yl)methoxy, 3-N-morpholinyl-propoxy, tetrahydrofuranoxy, dimethylamino, diethylamino, N-2-hydroxyethylamino, N-methyl-N-2-hydroxyethylamino, cyclopropylamino, cyclobutylamino, cyclopentylamino, cyclohexylamino, N-methylpyrrolidinyl-methylamino, N-acetylpyrrolidinyl-methylamino, (N-methyl)(N-methyl-oxet-3-yl)amino, dimethyl amino, pyrrolidin-2-on-1-yl, azetidinyl, 3,3-dimethylazetidinyl, 3-hydroxy-azetidinyl, 3-methoxy-azetidinyl, 3-ethoxy-azetidinyl, 3-propoxy-azetidinyl, 3-isopropoxy-azetidinyl, 3-(2-methoxyethoxy)azetidinyl, 3-(tert-butylsulfonyl)azetidinyl, 3-(optionally substituted phenyl)azetidinyl, pyrrolidinyl, pyrrolidin-2-one-1-yl, 3-hydroxypyrrolidinyl, 3-methoxypyrrolidinyl, 3,3-difluoropyrrolidinyl, 3,4-dihydroxypyrrolidin-1-yl, 3,4-dimethoxypyrrolidin-1-yl, piperidinyl, piperazinyl, morpholinyl, 1H-pyrrol-1-yl, 3 ,4-difluoro-1H-pyrrol-1-yl, pyrazol-1-yl, 4-methyl-1H-pyrazol-1-yl, 4-methoxy-1H-pyrazol-1-yl, 4-hydroxymethyl-1H-pyrazol-1-yl, 4-methoxymethyl-1H-pyrazol-1-yl, 1H-imidazol-1-yl, methyl-1H-imidazol-1-yl, N4-(C₁-C₆ alkyl)-piperazin-1-yl, N4-[SO₂(C₁-C₆ alkyl)]-piperazin-1-yl, imidazolyl, —C(═O)NH₂, —C(═O)NHCH₃, —C(═O)N(CH₃)₂, —NHC(═O)CH_{3, 2}-oxa-6-azaspiro[3.3]hept-6-yl, 2-oxa-6-azaspiro[3.3]hept-6-oxy, 2-azaspiro[3.3]hept-2-yl, 6-hydroxy-2-azaspiro[3.3]hept-2-yl, 2-oxaspiro[3.3]hept-6-yl, 2-oxa-6-azaspiro[3.4]oct-6-yl, 7-oxa-2-azaspiro[3.5]non-2-yl, 5,6-dimethoxy-1H-benzo[d]imidazyl, 1H-indazolyl, 6,7-difluoro-1-(3-methoxypropyl)-1H-indazolyl, 4-fluoro-2-(3-methoxypropyl)-2H-indazolyl, 7-fluoro-1-(3-methoxypropyl)-1H-indazolyl, 7-fluoro-2-(3-methoxypropyl)-2H-indazolyl, 4-fluoro-1-(3-methoxypropyl)-1H-indazolyl, benzo[d]thiazolyl, 2-methylbenzo[d]thiazolyl, 4,6-difluorobenzo[d]thiazolyl, 4-fluoro-2-methylbenzo[d]thiazolyl, 1H-benzo[d]imidazolyl, 4-fluoro-1-isopropyl-2-methyl-1H-benzo[d]imidazolyl, 7-fluoro-1-(3-methoxypropyl)-1H-benzo[d]imidazolyl, and 4-fluoro-1-(3-methoxypropyl)-1H-benzo[d]imidazolyl.

Embodiment 6 provides the compound of any of Embodiments 1-5, wherein R¹ is selected from the group consisting of:

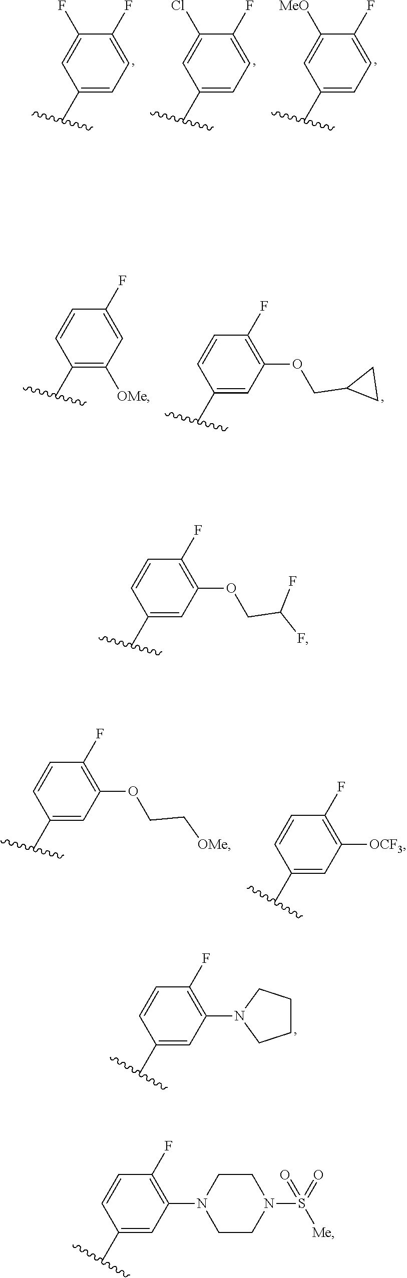

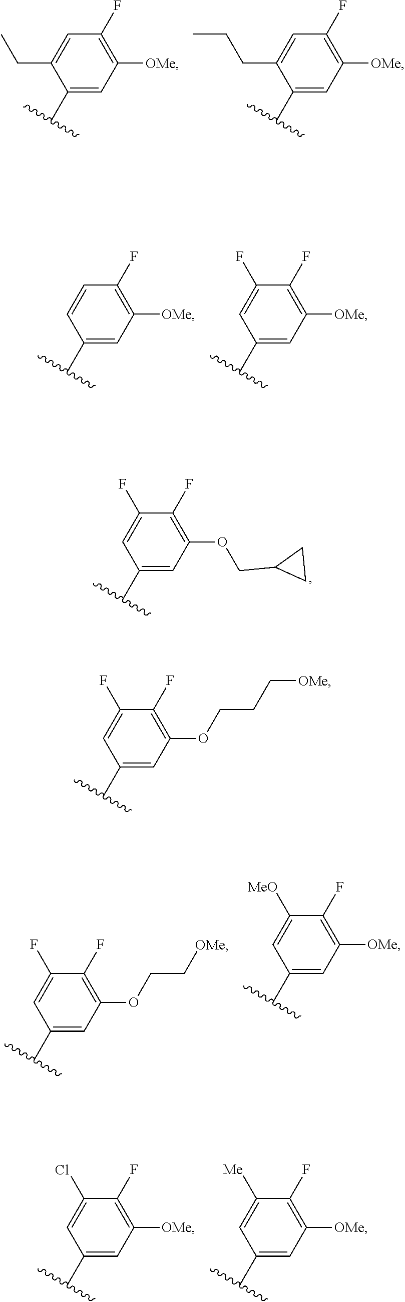

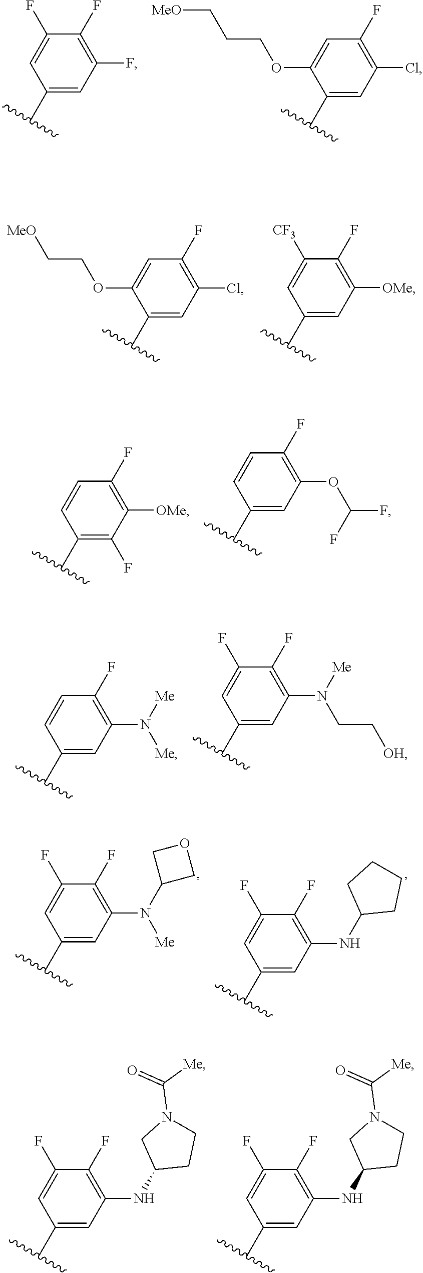

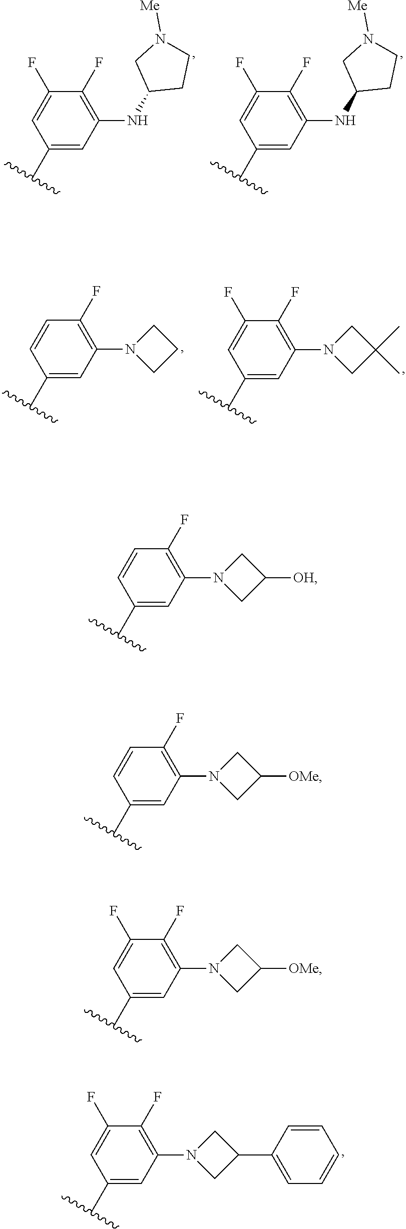

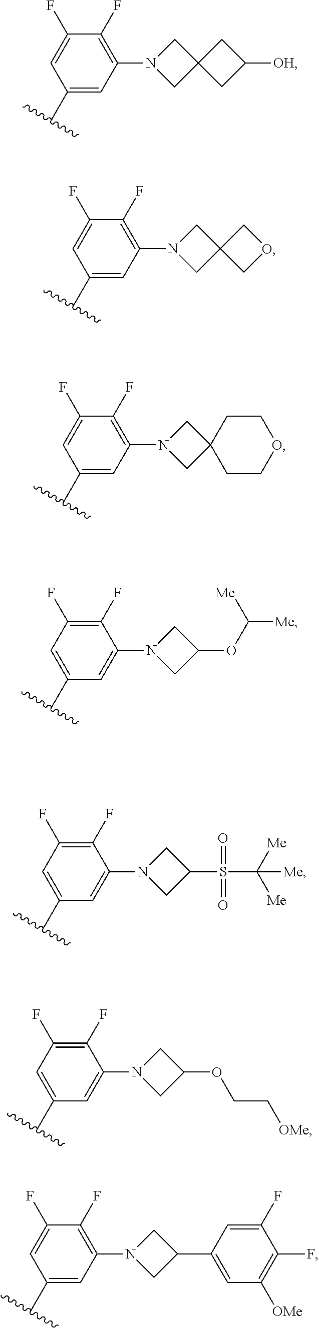

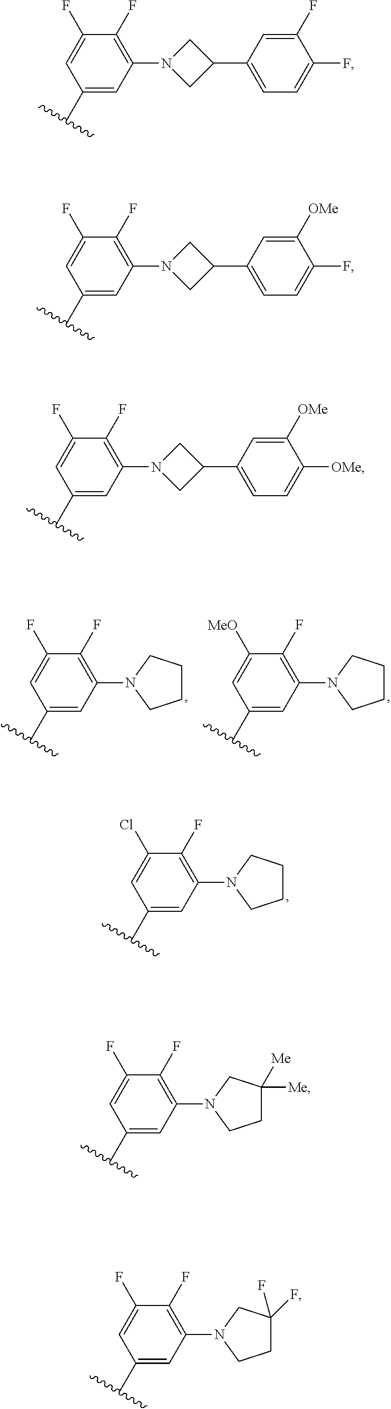

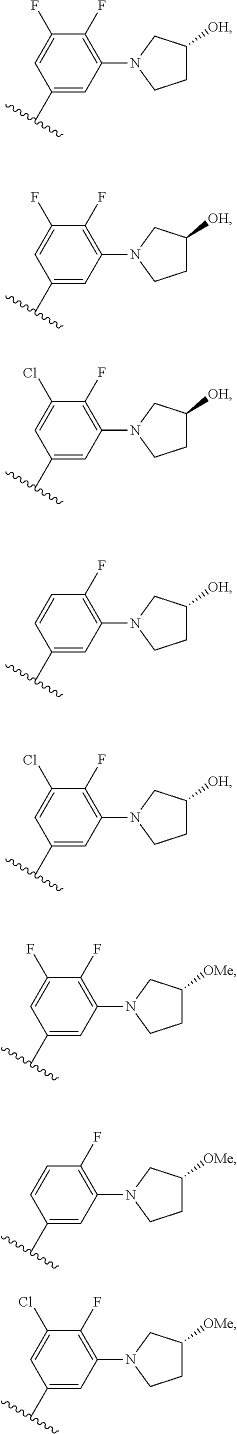

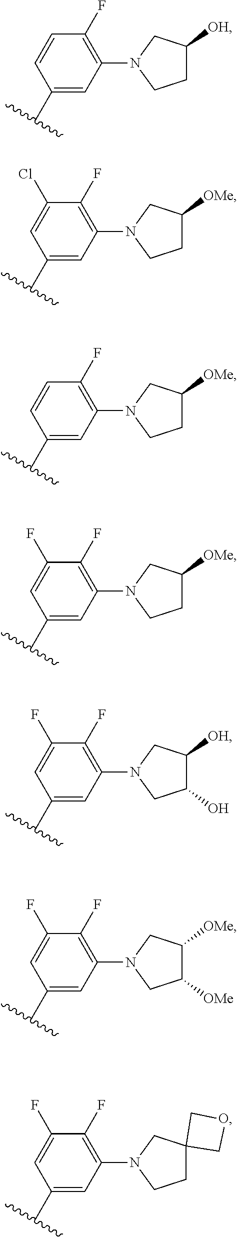

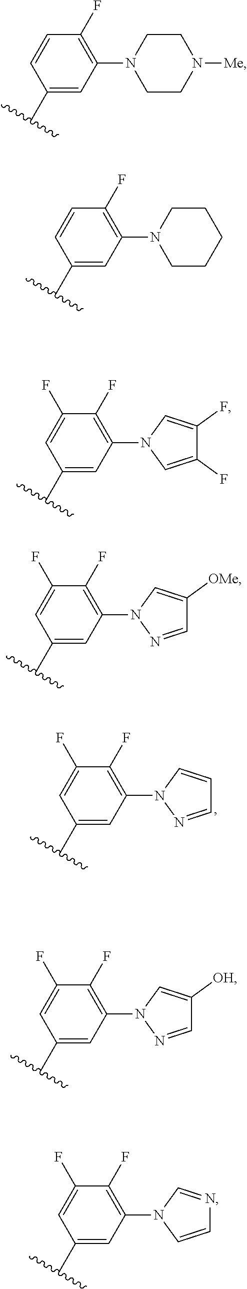

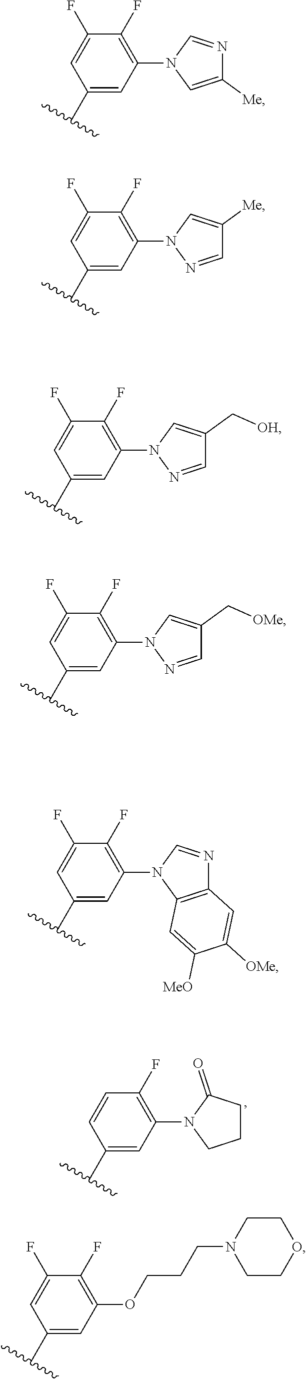

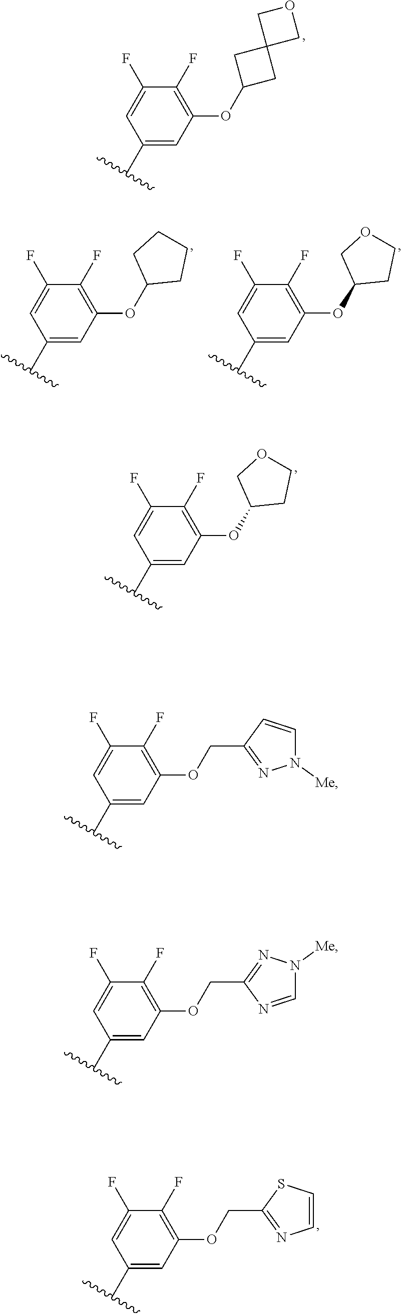

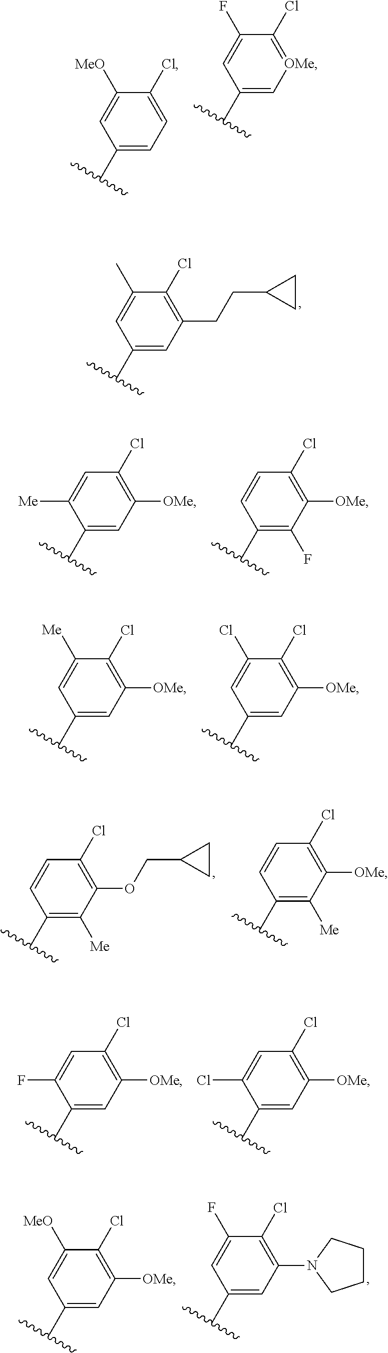

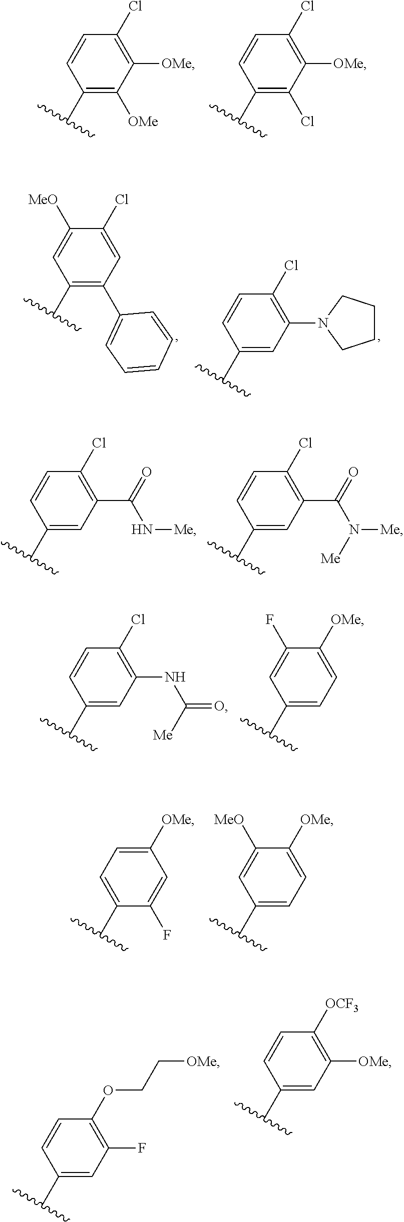

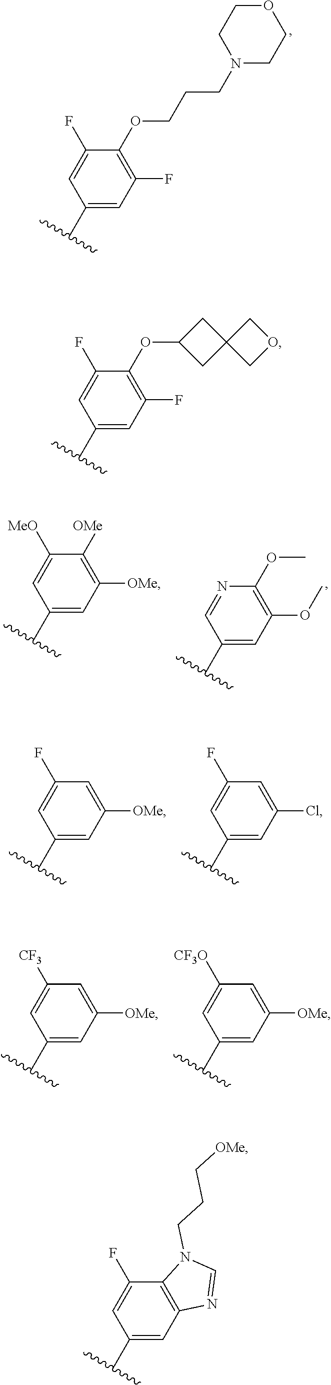

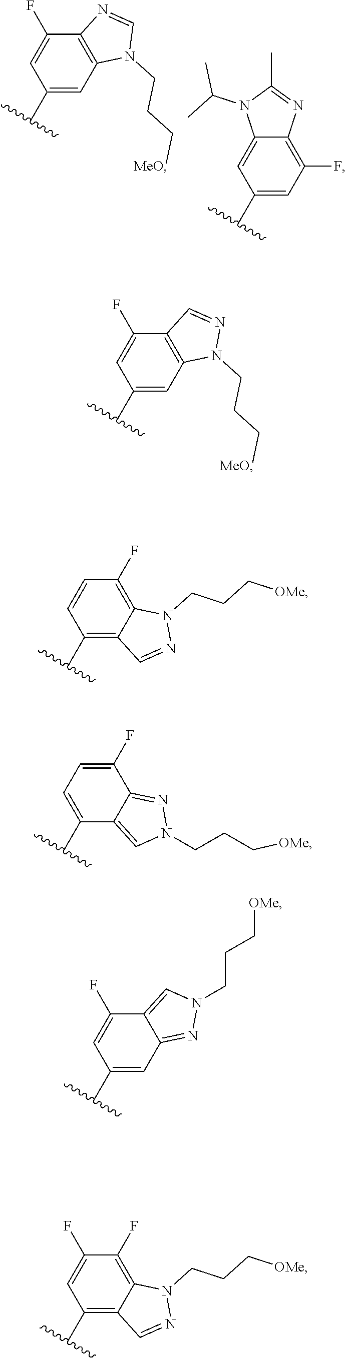

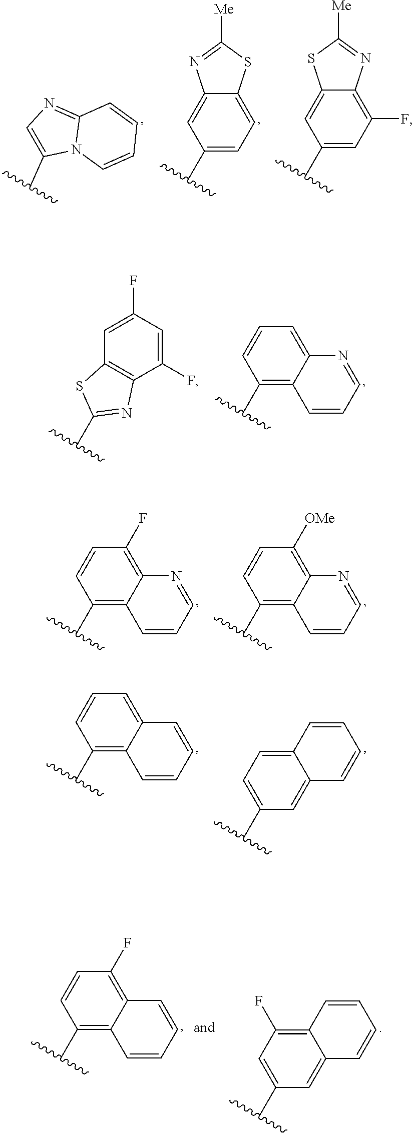

Embodiment 7 provides the compound of any of Embodiments 1-6, wherein each occurrence of R^{2a}, R^{2b}, R^{2c}, R^{2d}, and R^{2e} is independently selected from the group consisting of H, F, Cl, Br, I, methyl, ethyl, isopropyl, cyclopropyl, cyclobutyl, cyclopentyl, cyclohexyl, methoxy, ethoxy, isopropoxy, phenyl, optionally substituted benzo[d]thiazolyl, azetidinyl, pyrrolidinyl, piperidinyl, and piperazinyl. Embodiment 8 provides the compound of any of Embodiments 1-7, wherein each occurrence of R^{2a}, R^{2b}, R^{2c}, R^{2d}, and R^{2e} is independently selected from the group consisting of H, methyl, ethyl, isopropyl, cyclopropyl, cyclobutyl, cyclopentyl, cyclohexyl, methoxy, ethoxy, isopropoxy, phenyl, optionally substituted benzo[d]thiazolyl, azetidinyl, pyrrolidinyl, piperidinyl, and piperazinyl.

Embodiment 9 provides the compound of any of Embodiments 1-8, wherein each occurrence of R^{3a}, R^{3b}, R^{3c}, and R^{3d} is independently selected from the group consisting of H, halogen, cyano, nitro, optionally substituted C₁-C₆ alkyl, C₁-C₆ haloalkyl, optionally substituted C₃-C₈ cycloalkyl, C₁-C₆ haloalkoxy, C₁-C₆ hydroxyalkyl, and —OR″″, wherein each occurrence of R″″ is independently selected from the group consisting of H, optionally substituted C₁-C₆ alkyl, and optionally substituted C₃-C₈ cycloalkyl.

Embodiment 10 provides the compound of any of Embodiments 1-9, wherein each occurrence of R^{3b} is independently H, methyl, ethyl, propyl, cyclopropyl, isopropyl, methoxy, ethoxy, propoxy, cyclopropoxy, isopropoxy, fluoro, chloro, bromo, or iodo.

Embodiment 11 provides the compound of any of Embodiments 1-10, wherein each occurrence of alkyl, alkylenyl (alkylene), cycloalkyl, heterocyclyl, or carbocyclyl is independently optionally substituted with at least one substituent selected from the group consisting of C₁-C₆ alkyl, halogen, —OR″′, phenyl (thus yielding, in non-limiting examples, optionally substituted phenyl-(C₁-C₃ alkyl), —S(O)₂R″′, and —N(R″′)(R″′), wherein each occurrence of R″′ is independently H, optionally substituted C₁-C₆ alkyl, or optionally substituted C₃-C₈ cycloalkyl.

Embodiment 12 provides the compound of any of Embodiments 1-11, wherein each occurrence of aryl or heteroaryl is independently optionally substituted with at least one substituent selected from the group consisting of C₁-C₆ alkyl, C₁-C₆ haloalkyl, C₁-C₆ haloalkoxy, halogen, —CN, —OR″″, —N(R″″)(R″″), —NO₂, S(O)₂R″″, —S(═O)₂N(R″″)(R″″), acyl, and C₁-C₆ alkoxycarbonyl, wherein each occurrence of R″″ is independently H, optionally substituted C₁-C₆ alkyl, or optionally substituted C₃-C₈ cycloalkyl.

Embodiment 13 provides the compound of any of Embodiments 1-12, wherein each occurrence of aryl or heteroaryl is independently optionally substituted with at least one substituent selected from the group consisting of C₁-C₆ alkyl, C₁-C₆ haloalkyl, C₁-C₆ haloalkoxy, halogen, —CN, -OR″″, —N(R″″)(R″″), and C₁-C₆ alkoxycarbonyl, wherein each occurrence of R″″ is independently H, optionally substituted C₁-C₆ alkyl, or optionally substituted C₃-C₈ cycloalkyl.

Embodiment 14 provides the compound of any of Embodiments 1-13, which is selected from the group consisting of:

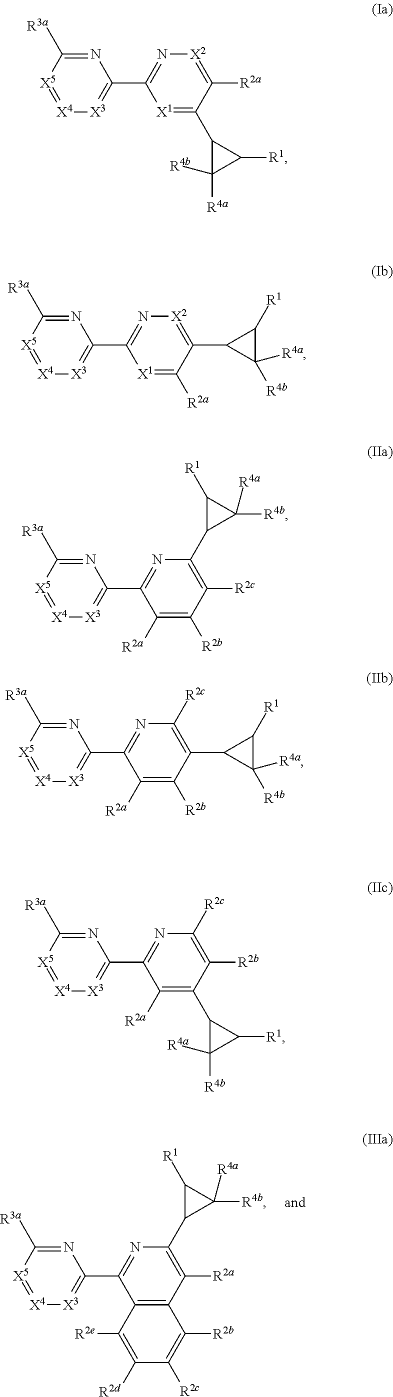

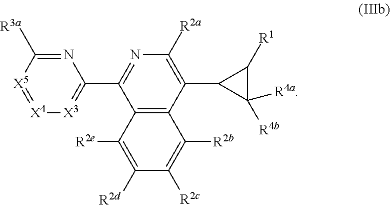

Embodiment 15 provides the compound of any of Embodiments 1-14, which is selected from the group consisting of:

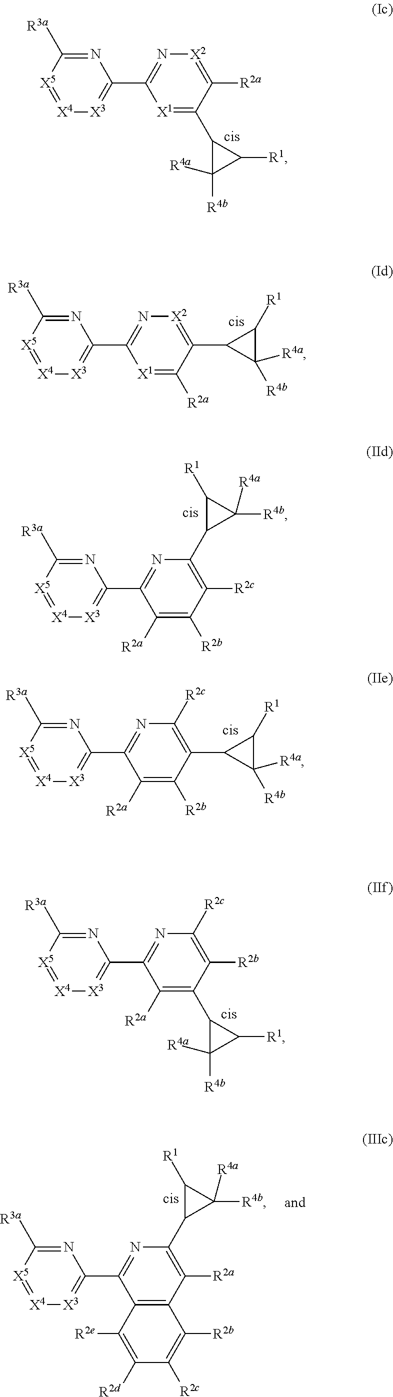

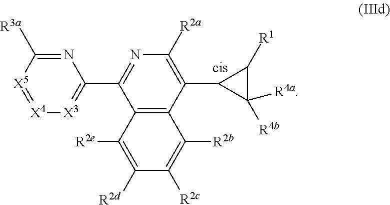

Embodiment 16 provides the compound of any of Embodiments 1-14, which is selected from the group consisting of:

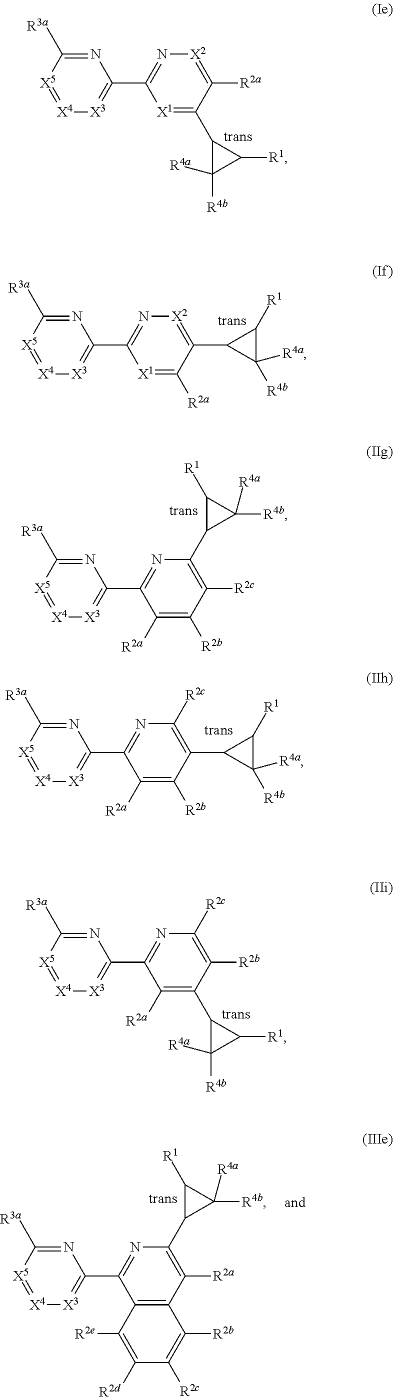

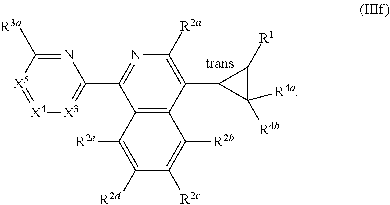

Embodiment 17 provides the compound of any of Embodiments 1-16, which is selected from the group consisting of:

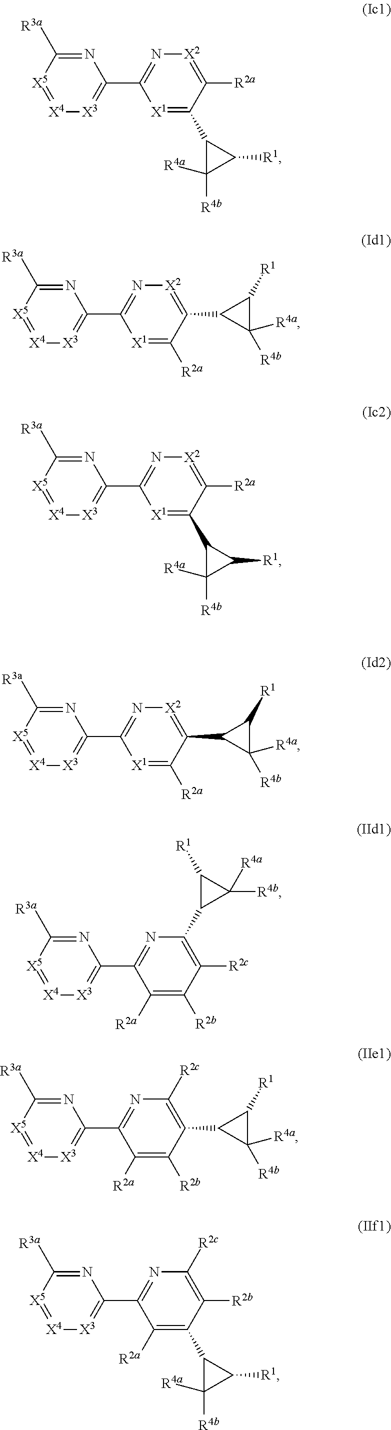

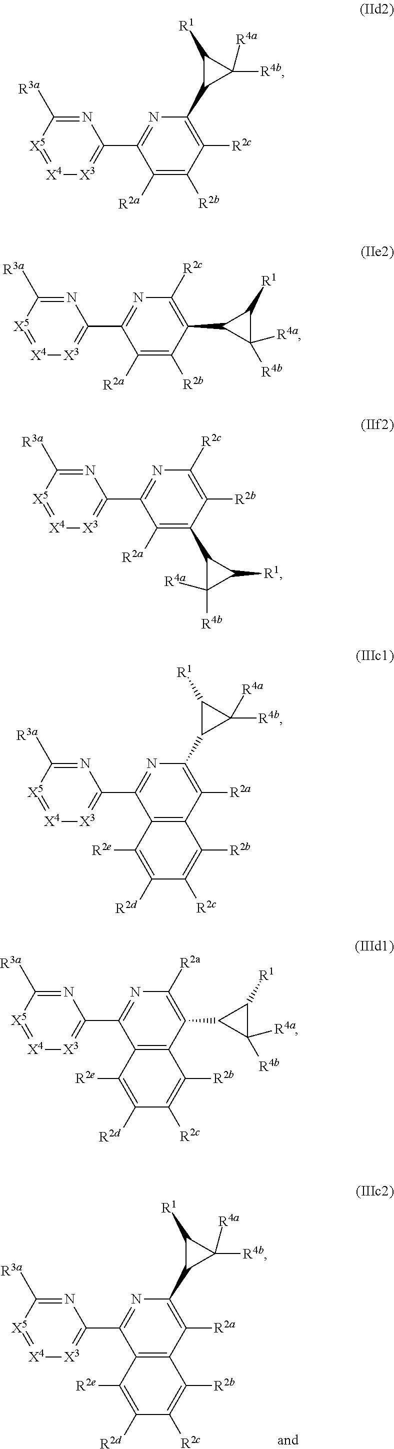

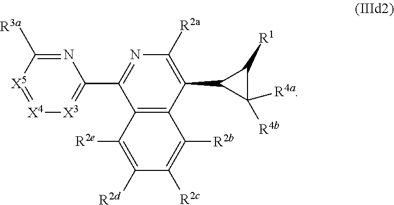

Embodiment 18 provides the compound of any of Embodiments 1-16, which is selected from the group consisting of:

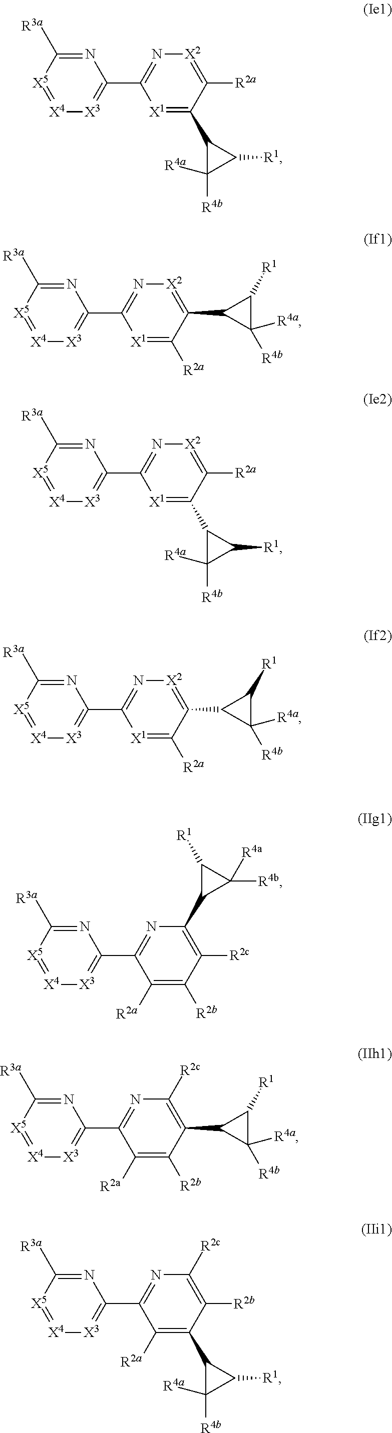

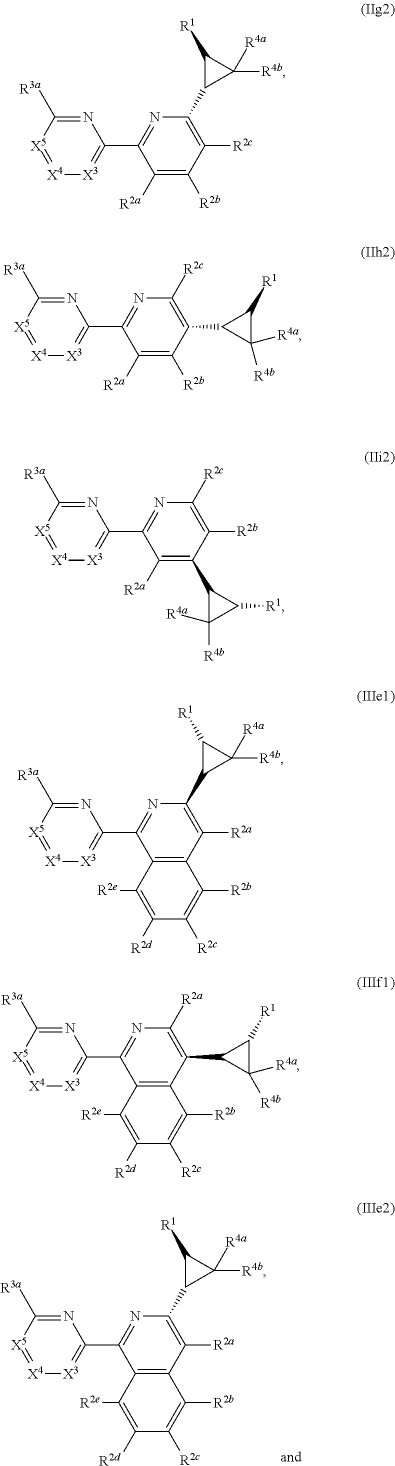

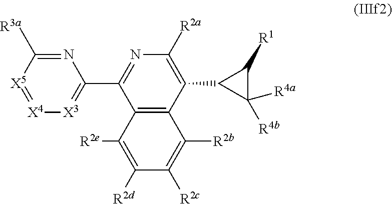

Embodiment 19 provides the compound of any of Embodiments 1-18, which is selected from the group consisting of:

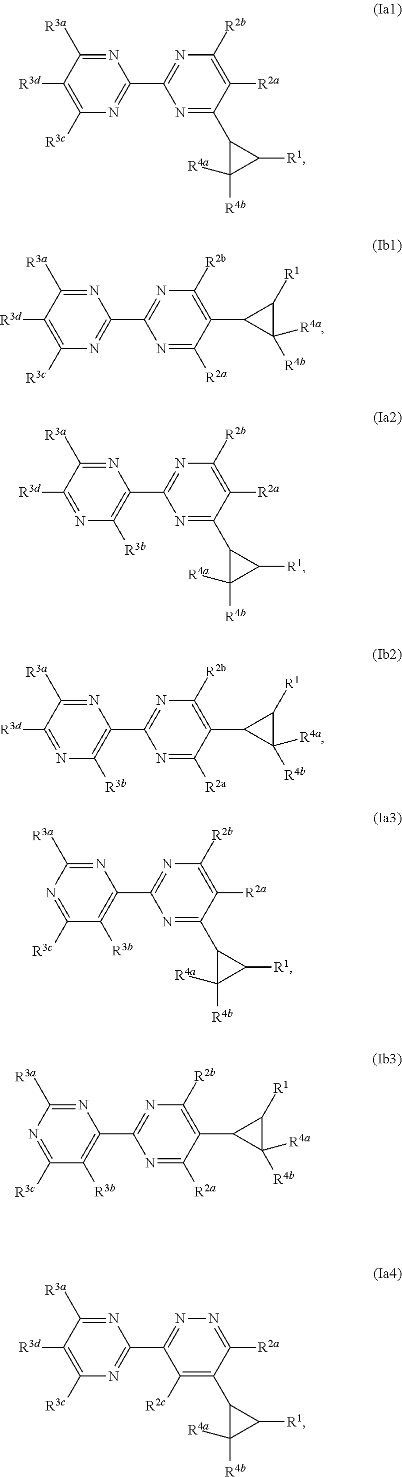

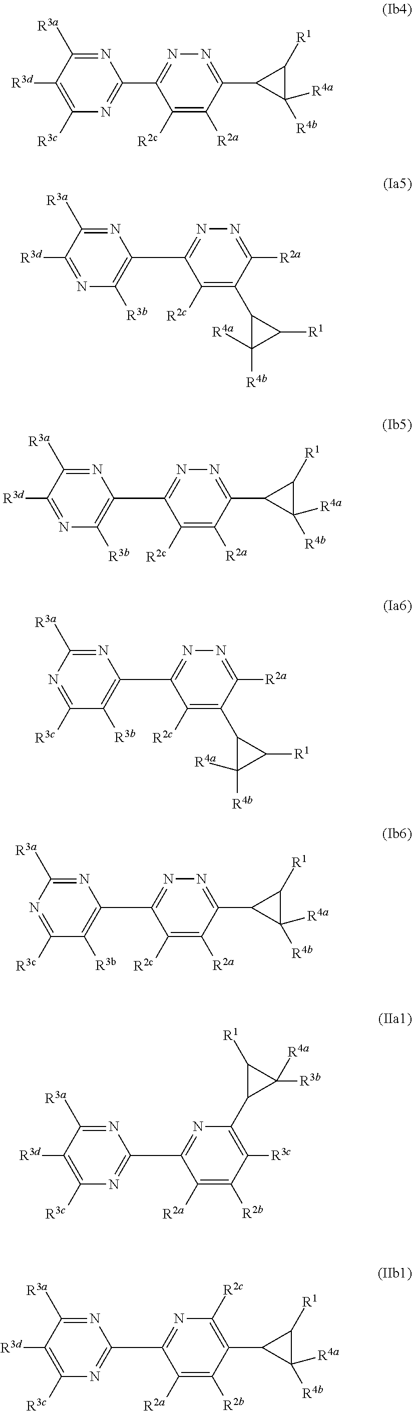

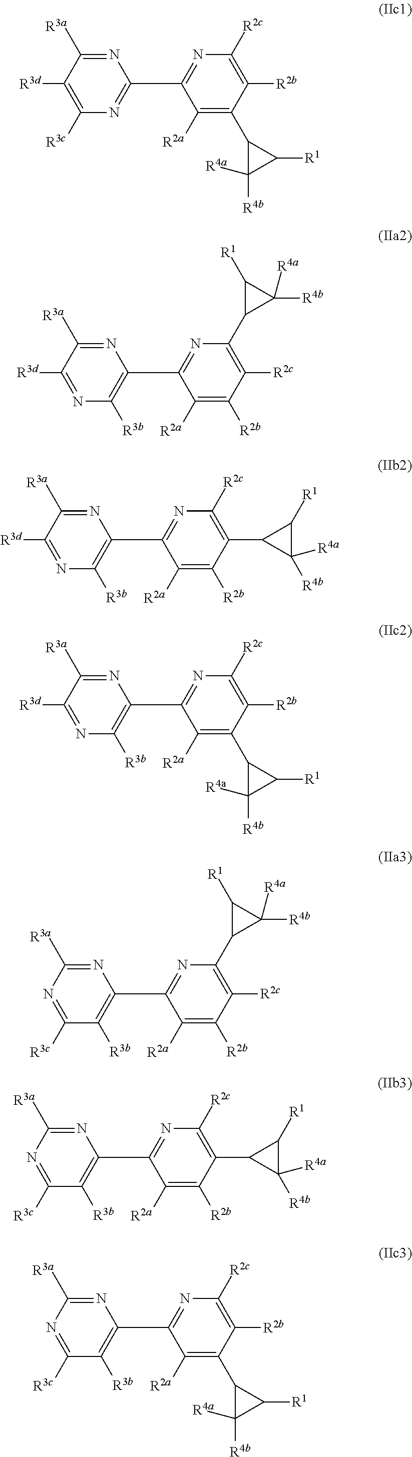

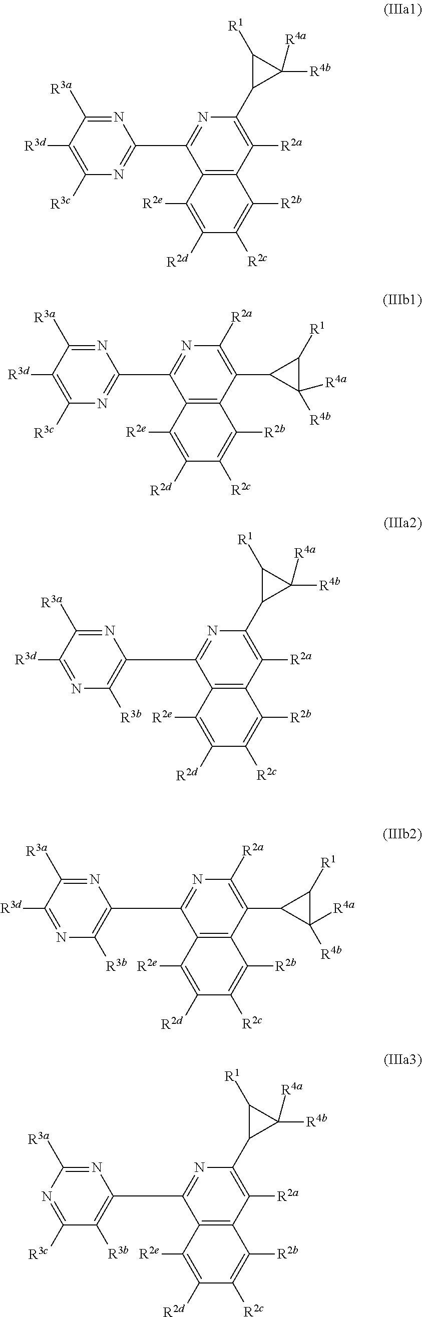

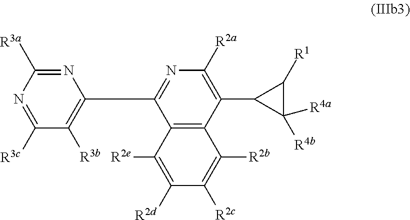

Embodiment 20 provides the compound of any of Embodiments 1-19, which is selected from the group consisting of:
5-(2-(2-fluoro-4-methoxyphenyl)cyclopropyl)-2,2′-bipyrimidine;
4-(2-(3,4-difluorophenyl)cyclopropyl)-2,2′-bipyrimidine;
5-(2-(4-fluoro-2-methoxyphenyl)cyclopropyl)-2,2′-bipyrimidine;
5-(2-(4-fluoro-3-methoxyphenyl)cyclopropyl)-2,2′-bipyrimidine;
5-(2-(3-fluoro-4-methoxyphenyl)cyclopropyl)-2,2′-bipyrimidine;
5-(2-(3,4-difluorophenyl)cyclopropyl)-2,2′-bipyrimidine;
5-(2-(4-fluoro-3-methoxyphenyl)cyclopropyl)-4-methyl-2,2′-bipyrimidine;
5-(2-(4-fluoro-3-(pyrrolidin-1-yl)phenyl)cyclopropyl)-2,2′-bipyrimidine;
5-(2-(4-fluoro-3-(2-methoxyethoxy)phenyl)cyclopropyl)-2,2′-bipyrimidine;
5-(2-(3-fluoro-4-(2-methoxyethoxy)phenyl)cyclopropyl)-2,2′-bipyrimidine;
5-(2-(3-(cyclopropylmethoxy)-4-fluorophenyl)cyclopropyl)-2,2′-bipyrimidine;
5-(2-(3-(2,2-difluoroethoxy)-4-fluorophenyl)cyclopropyl)-2,2′-bipyrimidine;
5-(2-(3-chloro-4-fluorophenyl)cyclopropyl)-2,2′-bipyrimidine;
5-(2-(3,4-dimethoxyphenyl)cyclopropyl)-2,2′-bipyrimidine;
5-(2-(4-chloro-3-methoxyphenyl)cyclopropyl)-2,2′-bipyrimidine;
5-(2-(2-ethyl-4-fluoro-5-methoxyphenyl)cyclopropyl)-2,2′-bipyrimidine;
5-(2-(4-fluoro-5-methoxy-2-propylphenyl)cyclopropyl)-2,2′-bipyrimidine;
5-(2-(4-fluoro-3-(trifluoromethoxy)phenyl)cyclopropyl)-2,2′-bipyrimidine;
5-(2-(4-fluoro-3-(4-(methylsulfonyl)piperazin-1-yl)phenyl)cyclopropyl)-2,2′-bipyrimidine;
5-(2-(3-(3,3-difluoropyrrolidin-1-yl)-4-fluorophenyl)cyclopropyl)-2,2′-bipyrimidine;
5-(2-(4-chloro-3-fluoro-5-methoxyphenyl)cyclopropyl)-2,2′-bipyrimidine;
5-(2-(4-fluoro-3-(3-methoxyazetidin-1-yl)phenyl)cyclopropyl)-2,2′-bipyrimidine;
5-((2-(4-chloro-3-(cyclopropylmethoxy)-5-methylphenyl)cyclopropyl)-2,2′-bipyrimidine;
5-(2-(4-chloro-5-methoxy-2-methylphenyl)cyclopropyl)-2,2′-bipyrimidine;
1-(5-(2-([2,2′-bipyrimidin]-5-yl)cyclopropyl)-2-fluorophenyl)azetidin-3-ol;
5-(2-(4-chloro-2-fluoro-3-methoxyphenyl)cyclopropyl)-2,2′-bipyrimidine;
5-(2-(4-chloro-3-methoxy-5-methylphenyl)cyclopropyl)-2,2′-bipyrimidine;
5-(2-(3,4-dichloro-5-methoxyphenyl)cyclopropyl)-2,2′-bipyrimidine;
5-(2-(4-chloro-3-(cyclopropylmethoxy)-2-methylphenyl)cyclopropyl)-2,2′-bipyrimidine;
5-(2-([2,2′-bipyrimidin]-5-yl)cyclopropyl)-2-fluoro-N,N-dimethylaniline;
(3S)-1-(5-(2-([2,2′-bipyrimidin]-5-yl)cyclopropyl)-2-fluorophenyl)pyrrolidin-3-ol;
5-(2-(4-fluoro-3-((S)-3-methoxypyrrolidin-1-yl)phenyl)cyclopropyl)-2,2′-bipyrimidine;
(3R)-1-(5-(2-([2,2′-bipyrimidin]-5-yl)cyclopropyl)-2-fluorophenyl)pyrrolidin-3-ol;
5-(2-(4-chloro-3-methoxy-2-methylphenyl)cyclopropyl)-2,2′-bipyrimidine;
5-(2-(4-fluoro-3-methoxyphenyl)cyclopropyl)-4-isopropyl-2,2′-bipyrimidine;
4-ethyl-5-(2-(4-fluoro-3-methoxyphenyl)cyclopropyl)-2,2′-bipyrimidine;
4-cyclohexyl-5-(2-(4-fluoro-3-methoxyphenyl)cyclopropyl)-2,2′-bipyrimidine;
5-(2-(4-fluoro-3-methoxyphenyl)cyclopropyl)-4-methoxy-2,2′-bipyrimidine;
5-(2-(3-(azetidin-1-yl)-4-fluorophenyl)cyclopropyl)-2,2′-bipyrimidine;
5-(2-(3,4-difluoro-5-methoxyphenyl)cyclopropyl)-2,2′-bipyrimidine;
5-(2-(4-chloro-2-fluoro-5-methoxyphenyl)cyclopropyl)-2,2′-bipyrimidine;
5-(2-(2,4-dichloro-5-methoxyphenyl)cyclopropyl)-2,2′-bipyrimidine;
5-(2-(4-chloro-3-fluoro-5-(pyrrolidin-1-yl)phenyl)cyclopropyl)-2,2′-bipyrimidine;
5-(2-(3,4-difluoro-5-(pyrrolidin-1-yl)phenyl)cyclopropyl)-2,2′-bipyrimidine;
5-(2-(4-fluoro-3-((R)-3-methoxypyrrolidin-1-yl)phenyl)cyclopropyl)-2,2′-bipyrimidine;
5-(2-(3,4-difluoro-5-((R)-3-methoxypyrrolidin-1-yl)phenyl)cyclopropyl)-2,2′-bipyrimidine;
5-(2-(4-chloro-3,5-dimethoxyphenyl)cyclopropyl)-2,2′-bipyrimidine;
5-(2-(4-fluoro-3-methoxy-5-(pyrrolidin-1-yl)phenyl)cyclopropyl)-2,2′-bipyrimidine;
5-(2-(3,4-difluoro-5-((S)-3-methoxypyrrolidin-1-yl)phenyl)cyclopropyl)-2,2′-bipyrimidine;
(3R)-1-(5-(2-([2,2′-bipyrimidin]-5-yl)cyclopropyl)-2,3-difluorophenyl)pyrrolidin-3-ol;
(3S)-1-(5-(2-([2,2′-bipyrimidin]-5-yl)cyclopropyl)-2,3-difluorophenyl)pyrrolidin-3-ol;
5-(2-(3-chloro-4-fluoro-5-(pyrrolidin-1-yl)phenyl)cyclopropyl)-2,2′-bipyrimidine;
5-(2-(3-chloro-4-fluoro-5-((R)-3-methoxypyrrolidin-1-yl)phenyl)cyclopropyl)-2,2′-bipyrimidine;
5-(2-(3-fluoro-5-methoxyphenyl)cyclopropyl)-2,2′-bipyrimidine;
5-(2-(4-chloro-2,3-dimethoxyphenyl)cyclopropyl)-2,2′-bipyrimidine;
5-(2-(3-chloro-4-fluoro-5-((S)-3-methoxypyrrolidin-1-yl)phenyl)cyclopropyl)-2,2′-bipyrimidine;
5-(2-(3-chloro-5-fluorophenyl)cyclopropyl)-2,2′-bipyrimidine;
(3S)-1-(5-(2-([2,2′-bipyrimidin]-5-yl)cyclopropyl)-3-chloro-2-fluorophenyl)pyrrolidin-3-ol;
5-(2-(3,4-difluoro-5-(3-methoxyazetidin-1-yl)phenyl)cyclopropyl)-2,2′-bipyrimidine;
2-(5-(2-(3,4-difluoro-5-methoxyphenyl)cyclopropyl)pyridin-2-yl)pyrimidine;
(3R)-1-(5-(2-([2,2′-bipyrimidin]-5-yl)cyclopropyl)-3-chloro-2-fluorophenyl)pyrrolidin-3-ol;
5-(2-(3-(cyclopropylmethoxy)-4,5-difluorophenyl)cyclopropyl)-2,2′-bipyrimidine;
5-(2-(3,4-difluoro-5-(3-methoxypropoxy)phenyl)cyclopropyl)-2,2′-bipyrimidine;
5-(2-(2,4-dichloro-3-methoxyphenyl)cyclopropyl)-2,2′-bipyrimidine;
5-(2-(3,4-difluoro-5-(2-methoxyethoxy)phenyl)cyclopropyl)-2,2′-bipyrimidine;
2-(5-(2-([2,2′-bipyrimidin]-5-yl)cyclopropyl)-2,3-difluorophenyl)-7-oxa-2-azaspiro[3.5]nonane;
5-(2-(3-(3,3-dimethylazetidin-1-yl)-4,5-difluorophenyl)cyclopropyl)-2,2′-bipyrimidine;
6-(5-(2-(4-fluoro-3-methoxyphenyl)cyclopropyl)-[2,2′-bipyrimidin]-4-yl)-2-methylbenzo[d]thiazole;
5-(2-(4-Fluoro-3-methoxyphenyl)cyclopropyl)-4-phenyl-2,2′-bipyrimidine;
5-(2-(4-fluoro-3-methoxyphenyl)cyclopropyl)-4-(piperidin-1-yl)-2,2′-bipyrimidine;
5-(2-(4-fluoro-3,5-dimethoxyphenyl)cyclopropyl)-2,2′-bipyrimidine;
5-(2-(3-chloro-4-fluoro-5-methoxyphenyl)cyclopropyl)-2,2′-bipyrimidine;
5-(2-(4-fluoro-3-methoxy-5-methylphenyl)cyclopropyl)-2,2′-bipyrimidine;
5-(2-(3-methoxy-4-(trifluoromethoxy)phenyl)cyclopropyl)-2,2′-bipyrimidine;
5-(2-(3-methoxy-4-(trifluoromethyl)phenyl)cyclopropyl)-2,2′-bipyrimidine;
5-(2-(5-chloro-4-fluoro-2-(3-methoxypropoxy)phenyl)cyclopropyl)-2,2′-bipyrimidine;
5-(2-(5-chloro-4-fluoro-2-(2-methoxyethoxy)phenyl)cyclopropyl)-2,2′-bipyrimidine;
5-(2-(5-chloro-4-methoxy-[1,1′-biphenyl]-2-yl)cyclopropyl)-2,2′-bipyrimidine;
5-(2-(3,4,5-trifluorophenyl)cyclopropyl)-2,2′-bipyrimidine;
5-(2-(3-methoxy-5-(trifluoromethyl)phenyl)cyclopropyl)-2,2′-bipyrimidine;
5-(2-(3-methoxy-5-(trifluoromethoxy)phenyl)cyclopropyl)-2,2′-bipyrimidine;
5-(2-(4-fluoro-3-methoxy-5-(trifluoromethyl)phenyl)cyclopropyl)-2,2′-bipyrimidine;
5-(2-(naphthalen-1-yl)cyclopropyl)-2,2′-bipyrimidine;
5-(2-(naphthalen-2-yl)cyclopropyl)-2,2′-bipyrimidine;
5-(2-(4-fluoronaphthalen-1-yl)cyclopropyl)-2,2′-bipyrimidine;
5-(2-(4-fluoronaphthalen-2-yl)cyclopropyl)-2,2′-bipyrimidine;
3-(2-([2,2′-bipyrimidin]-5-yl)cyclopropyl)imidazo[1,2-a]pyridine;
5-(2-([2,2′-bipyrimidin]-5-yl)cyclopropyl)quinoline;
5-(2-([2,2′-bipyrimidin]-5-yl)cyclopropyl)-8-fluoroquinoline;
5-(2-([2,2′-bipyrimidin]-5-yl)cyclopropyl)-8-methoxyquinoline;
5-(2-(3,4-difluoro-5-methoxyphenyl)cyclopropyl)-2-(pyridin-2-yl)pyrimidine;
5-(2-(4-fluoro-3,5-dimethoxyphenyl)cyclopropyl)-2-(pyridin-2-yl)pyrimidine;
2-(pyridin-2-yl)-5-(2-(3,4,5-trimethoxyphenyl)cyclopropyl)pyrimidine;
5-(2-(3,4-difluoro-5-methoxyphenyl)cyclopropyl)-2,4′-bipyrimidine;
5-(2-(3,4-difluoro-5-methoxyphenyl)cyclopropyl)-2-(pyrazin-2-yl)pyrimidine;
5-(2-(3,4-difluoro-5-methoxyphenyl)cyclopropyl)-2-(3-fluoropyridin-2-yl)pyrimidine;
5-(2-(3,4-difluoro-5-methoxyphenyl)cyclopropyl)-2-(3-methoxypyridin-2-yl)pyrimidine;
5-(2-(3,4-difluoro-5-(1H-imidazol-1-yl)phenyl)cyclopropyl)-2,2′-bipyrimidine;
5-(2-(5,6-dimethoxypyridin-3-yl)cyclopropyl)-2,2′-bipyrimidine;
5-(2-(3-(difluoromethoxy)-4-fluorophenyl)cyclopropyl)-2,2′-bipyrimidine;
5-(2-(4-fluoro-3-(4-methylpiperazin-1-yl)phenyl)cyclopropyl)-2,2′-bipyrimidine;
5-(2-(2,4-difluoro-3-methoxyphenyl)cyclopropyl)-2,2′-bipyrimidine;
3-(2-(4-fluoro-3-methoxyphenyl)cyclopropyl)-6-(pyrimidin-2-yl)pyridazine;
4-(2-(4-fluoro-3-methoxyphenyl)cyclopropyl)-1-(pyrimidin-2-yl)isoquinoline;
5-(2-(4-fluoro-3-(piperidin-1-yl)phenyl)cyclopropyl)-2,2′-bipyrimidine;
1-(5-(2-([2,2′-bipyrimidin]-5-yl)cyclopropyl)-2-fluorophenyl)pyrrolidin-2-one;
5-(2-(4-chloro-3-(pyrrolidin-1-yl)phenyl)cyclopropyl)-2,2′-bipyrimidine;
5-((2-([2,2′-bipyrimidin]-5-yl)cyclopropyl)-2-chloro-N-methylbenzamide;
5-(2-([2,2′-bipyrimidin]-5-yl)cyclopropyl)-2-chloro-N,N-dimethylbenzamide;
N-(5-(2-([2,2′-bipyrimidin]-5-yl)cyclopropyl)-2-chlorophenyl)acetamide;
5-(2-([2,2′-bipyrimidin]-5-yl)cyclopropyl)-N-cyclopentyl-2,3-difluoroaniline;
6-(5-(2-([2,2′-bipyrimidin]-5-yl)cyclopropyl)-2,3-difluorophenyl)-2-oxa-6-azaspiro[3.3]heptane;
5-(2-(3,4-difluoro-5-(3-isopropoxyazetidin-1-yl)phenyl)cyclopropyl)-2,2′-bipyrimidine;
5-(2-(3-(3-(tert-butyl sulfonyl)azetidin-1-yl)-4,5-difluorophenyl)cyclopropyl)-2,2′-bipyrimidine;
5-(2-(3,4-difluoro-5-(3-(2-methoxyethoxy)azetidin-1-yl)phenyl)cyclopropyl)-2,2′-bipyrimidine;
5-(2-(3-(3-(3,4-difluoro-5-methoxyphenyl)azetidin-1-yl)-4,5-difluorophenyl)cyclopropyl)-2,2′-bipyrimidine;
5-(2-(3-(3-(3,4-difluorophenyl)azetidin-1-yl)-4,5-difluorophenyl)cyclopropyl)-2,2′-bipyrimidine;
5-(2-(3,4-difluoro-5-(3-(4-fluoro-3-methoxyphenyl)azetidin-1-yl)phenyl)cyclopropyl)-2,2′-bipyrimidine;
5-(2-(3-(3-(3,4-dimethoxyphenyl)azetidin-1-yl)-4,5-difluorophenyl)cyclopropyl)-2,2′-bipyrimidine;
5-(2-(3,4-difluoro-5-((1-methyl-1H-1,2,4-triazol-3-yl)methoxy)phenyl)cyclopropyl)-2,2′-bipyrimidine;
2-((5-(2-([2,2′-bipyrimidin]-5-yl)cyclopropyl)-2,3-difluorophenoxy)methyl)thiazole;
5-(2-(3,4-difluoro-5-((1-methyl-1H-pyrazol-3-yl)methoxy)phenyl)cyclopropyl)-2,2′-bipyrimidine;
5-(2-(3-(3,3-dimethylpyrrolidin-1-yl)-4,5-difluorophenyl)cyclopropyl)-2,2′-bipyrimidine;
6-(5-(2-([2,2′-bipyrimidin]-5-yl)cyclopropyl)-2,3-difluorophenyl)-2-oxa-6-azaspiro[3.4]octane;
1-((3S)-3-((5-(2-([2,2′-bipyrimidin]-5-yl)cyclopropyl)-2,3-difluorophenyl)amino)pyrrolidin-1-yl)ethenone;
1-((3R)-3-((5-(2-([2,2′-bipyrimidin]-5-yl)cyclopropyl)-2,3-difluorophenyl) amino) pyrrolidin-1-yl)ethenone;
(3S)-N-(5-(2-([2,2′-bipyrimidin]-5-yl)cyclopropyl)-2,3-difluorophenyl)-1-methylpyrrolidin-3-amine;
(3R)-N-(5-(2-([2,2′-bipyrimidin]-5-yl)cyclopropyl)-2,3-difluorophenyl)-1-methylpyrrolidin-3-amine;
5-(2-(3,4-difluoro-5-(4-methyl-1H-pyrazol-1-yl)phenyl)cyclopropyl)-2,2′-bipyrimidine;
N-(5-(2-([2,2′-bipyrimidin]-5-yl)cyclopropyl)-2,3-difluorophenyl)-N-methyloxetan-3-amine;
(1-(5-(2-([2,2′-bipyrimidin]-5-yl)cyclopropyl)-2,3-difluorophenyl)-1H-pyrazol-4-yl)methanol;
5-(2-(3-((3R,4 S)-3,4-dimethoxypyrrolidin-1-yl)-4,5-difluorophenyl)cyclopropyl)-2,2′-bipyrimidine;
5-(2-(3,4-difluoro-5-(4-(methoxymethyl)-1H-pyrazol-1-yl)phenyl)cyclopropyl)-2,2′-bipyrimidine;
1-(5-(2-([2,2′-bipyrimidin]-5-yl)cyclopropyl)-2,3-difluorophenyl)-5,6-dimethoxy-1H-benzo[d]imidazole;
2-(5-(2-([2,2′-bipyrimidin]-5-yl)cyclopropyl)-2,3-difluorophenyl)-2-azaspiro[3.3]heptan-6-ol;
(3R,4R)-1-(5-(2-([2,2′-bipyrimidin]-5-yl)cyclopropyl)-2,3-difluorophenyl)pyrrolidine-3,4-diol;
4-(3-(5-(2-([2,2′-bipyrimidin]-5-yl)cyclopropyl)-2,3-difluorophenoxy)propyl)morpholine;
4-(3-(4-(2-([2,2′-bipyrimidin]-5-yl)cyclopropyl)-2,6-difluorophenoxy)propyl)morpholine;
5-(2-(4-((2-oxaspiro[3.3]heptan-6-yl)oxy)-3,5-difluorophenyl)cyclopropyl)-2,2′-bipyrimidine;
5-(2-(3-((2-oxaspiro[3.3]heptan-6-yl)oxy)-4,5-difluorophenyl)cyclopropyl)-2,2′-bipyrimidine;
2-((5-(2-([2,2′-bipyrimidin]-5-yl)cyclopropyl)-2,3-difluorophenyl)(methyl)amino)ethan-1-ol;
5-(2-(3-(cyclopentyloxy)-4,5-difluorophenyl)cyclopropyl)-2,2′-bipyrimidine;
5-(2-(3,4-difluoro-5-(((R)-tetrahydrofuran-3-yl)oxy)phenyl)cyclopropyl)-2,2′-bipyrimidine;
5-(2-(3,4-difluoro-5-(((S)-tetrahydrofuran-3-yl)oxy)phenyl)cyclopropyl)-2,2′-bipyrimidine;
5-(2-(3,4-difluoro-5-(4-methoxy-1H-pyrazol-1-yl)phenyl)cyclopropyl)-2,2′-bipyrimidine;
5-(2-(3,4-difluoro-5-(1H-pyrazol-1-yl)phenyl)cyclopropyl)-2,2′-bipyrimidine;
5-(2-(3,4-difluoro-5-(3-phenylazetidin-1-yl)phenyl)cyclopropyl)-2,2′-bipyrimidine;
5-(2-(3-(3,4-difluoro-1H-pyrrol-1-yl)-4,5-difluorophenyl)cyclopropyl)-2,2′-bipyrimidine;
1-(5-(2-([2,2′-bipyrimidin]-5-yl)cyclopropyl)-2,3-difluorophenyl)-1H-pyrazol-4-ol;
5-(2-(3,4-difluoro-5-(4-methyl-1H-imidazol-1-yl)phenyl)cyclopropyl)-2,2′-bipyrimidine;
ethyl 2-([2,2′-bipyrimidin]-5-yl)-3-(3,4-difluoro-5-methoxyphenyl)cyclopropane-1-carboxylate;
4-(2-([2,2′-bipyrimidin]-5-yl)cyclopropyl)-6,7-difluoro-1-(3-methoxypropyl)-1H-indazole;
6-(2-([2,2′-bipyrimidin]-5-yl)cyclopropyl)-4-fluoro-2-(3-methoxypropyl)-2H-indazole;
2-(2-([2,2′-bipyrimidin]-5-yl)cyclopropyl)-4,6-difluorobenzo[d]thiazole;
6-(2-([2,2′-bipyrimidin]-5-yl)cyclopropyl)-4-fluoro-1-isopropyl-2-methyl-1H-benzo[d]imidazole;
6-(2-([2,2′-bipyrimidin]-5-yl)cyclopropyl)-4-fluoro-2-methylbenzo[d]thiazole;
5-(2-([2,2′-bipyrimidin]-5-yl)cyclopropyl)-7-fluoro-1-(3-methoxypropyl)-1H-benzo[d]imidazole;
6-(2-([2,2′-bipyrimidin]-5-yl)cyclopropyl)-4-fluoro-1-(3-methoxypropyl)-1H-benzo[d]imidazole;
6-(2-([2,2′-bipyrimidin]-5-yl)cyclopropyl)-4-fluoro-1-(3-methoxypropyl)-1H-indazole;
4-(2-([2,2′-bipyrimidin]-5-yl)cyclopropyl)-7-fluoro-1-(3-methoxypropyl)-1H-indazole;
4-(2-([2,2′-bipyrimidin]-5-yl)cyclopropyl)-7-fluoro-2-(3-methoxypropyl)-2H-indazole; and
5-(2-([2,2′-bipyrimidin]-5-yl)cyclopropyl)-2-methylbenzo[d]thiazole.

Embodiment 21 provides the compound of any of Embodiments 1-20, which is selected from the group consisting of:
trans-5-(2-(2-fluoro-4-methoxyphenyl)cyclopropyl)-2,2′-bipyrimidine;
trans-4-(2-(3,4-difluorophenyl)cyclopropyl)-2,2′-bipyrimidine;
trans-5-(2-(4-fluoro-2-methoxyphenyl)cyclopropyl)-2,2′-bipyrimidine;
trans-5-(2-(4-fluoro-3-methoxyphenyl)cyclopropyl)-2,2′-bipyrimidine;
trans-5-(2-(3-fluoro-4-methoxyphenyl)cyclopropyl)-2,2′-bipyrimidine;
trans-5-(2-(3,4-difluorophenyl)cyclopropyl)-2,2′-bipyrimidine;
trans-5-(2-(4-fluoro-3-methoxyphenyl)cyclopropyl)-4-methyl-2,2′-bipyrimidine;
trans-5-(2-(4-fluoro-3-(pyrrolidin-1-yl)phenyl)cyclopropyl)-2,2′-bipyrimidine;
trans-5-(2-(4-fluoro-3-(2-methoxyethoxy)phenyl)cyclopropyl)-2,2′-bipyrimidine;
trans-5-(2-(3-fluoro-4-(2-methoxyethoxy)phenyl)cyclopropyl)-2,2′-bipyrimidine;
trans-5-(2-(3-(cyclopropylmethoxy)-4-fluorophenyl)cyclopropyl)-2,2′-bipyrimidine;
trans-5-(2-(3-(2,2-difluoroethoxy)-4-fluorophenyl)cyclopropyl)-2,2′-bipyrimidine;
trans-5-(2-(3-chloro-4-fluorophenyl)cyclopropyl)-2,2′-bipyrimidine;
trans-5-(2-(3,4-dimethoxyphenyl)cyclopropyl)-2,2′-bipyrimidine;
trans-5-(2-(4-chloro-3-methoxyphenyl)cyclopropyl)-2,2′-bipyrimidine;
trans-5-(2-(2-ethyl-4-fluoro-5-methoxyphenyl)cyclopropyl)-2,2′-bipyrimidine;
trans-5-(2-(4-fluoro-5-methoxy-2-propylphenyl)cyclopropyl)-2,2′-bipyrimidine;
trans-5-(2-(4-fluoro-3-(trifluoromethoxy)phenyl)cyclopropyl)-2,2′-bipyrimidine;
trans-5-(2-(4-fluoro-3-(4-(methylsulfonyl)piperazin-1-yl)phenyl)cyclopropyl)-2,2′-bipyrimidine;
trans-5-(2-(3-(3,3-difluoropyrrolidin-1-yl)-4-fluorophenyl)cyclopropyl)-2,2′-bipyrimidine;
trans-5-(2-(4-chloro-3-fluoro-5-methoxyphenyl)cyclopropyl)-2,2′-bipyrimidine;
trans-5-(2-(4-fluoro-3-(3-methoxyazetidin-1-yl)phenyl)cyclopropyl)-2,2′-bipyrimidine;
trans-5-((2-(4-chloro-3-(cyclopropylmethoxy)-5-methylphenyl)cyclopropyl)-2,2′-bipyrimidine;
trans-5-(2-(4-chloro-5-methoxy-2-methylphenyl)cyclopropyl)-2,2′-bipyrimidine;
trans-1-(5-(2-([2,2′-bipyrimidin]-5-yl)cyclopropyl)-2-fluorophenyl)azetidin-3-ol;
trans-5-(2-(4-chloro-2-fluoro-3-methoxyphenyl)cyclopropyl)-2,2′-bipyrimidine;
trans-5-(2-(4-chloro-3-methoxy-5-methylphenyl)cyclopropyl)-2,2′-bipyrimidine;
trans-5-(2-(3,4-dichloro-5-methoxyphenyl)cyclopropyl)-2,2′-bipyrimidine;
trans-5-(2-(4-chloro-3-(cyclopropylmethoxy)-2-methylphenyl)cyclopropyl)-2,2′-bipyrimidine;
trans-5-(2-([2,2′-bipyrimidin]-5-yl)cyclopropyl)-2-fluoro-N,N-dimethylaniline;
trans-(3S)-1-(5-(2-([2,2′-bipyrimidin]-5-yl)cyclopropyl)-2-fluorophenyl)pyrrolidin-3-ol;
trans-5-(2-(4-fluoro-3-((S)-3-methoxypyrrolidin-1-yl)phenyl)cyclopropyl)-2,2′-bipyrimidine;
trans-(3R)-1-(5-(2-([2,2′-bipyrimidin]-5-yl)cyclopropyl)-2-fluorophenyl)pyrrolidin-3-ol;
trans-5-(2-(4-chloro-3-methoxy-2-methylphenyl)cyclopropyl)-2,2′-bipyrimidine;
trans-5-(2-(4-fluoro-3-methoxyphenyl)cyclopropyl)-4-isopropyl-2,2′-bipyrimidine;
trans-4-ethyl-5-(2-(4-fluoro-3-methoxyphenyl)cyclopropyl)-2,2′-bipyrimidine;
trans-4-cyclohexyl-5-(2-(4-fluoro-3-methoxyphenyl)cyclopropyl)-2,2′-bipyrimidine;
trans-5-(2-(4-fluoro-3-methoxyphenyl)cyclopropyl)-4-methoxy-2,2′-bipyrimidine;
trans-5-(2-(3-(azetidin-1-yl)-4-fluorophenyl)cyclopropyl)-2,2′-bipyrimidine;
trans-5-(2-(3,4-difluoro-5-methoxyphenyl)cyclopropyl)-2,2′-bipyrimidine;
trans-5-(2-(4-chloro-2-fluoro-5-methoxyphenyl)cyclopropyl)-2,2′-bipyrimidine;
trans-5-(2-(2,4-dichloro-5-methoxyphenyl)cyclopropyl)-2,2′-bipyrimidine;
trans-5-(2-(4-chloro-3-fluoro-5-(pyrrolidin-1-yl)phenyl)cyclopropyl)-2,2′-bipyrimidine;
trans-5-(2-(3,4-difluoro-5-(pyrrolidin-1-yl)phenyl)cyclopropyl)-2,2′-bipyrimidine;
trans-5-(2-(4-fluoro-34(R)-3-methoxypyrrolidin-1-yl)phenyl)cyclopropyl)-2,2′-bipyrimidine;
trans-5-(2-(3,4-difluoro-5-((R)-3-methoxypyrrolidin-1-yl)phenyl)cyclopropyl)-2,2′-bipyrimidine;
trans-5-(2-(4-chloro-3,5-dimethoxyphenyl)cyclopropyl)-2,2′-bipyrimidine;
trans-5-(2-(4-fluoro-3-methoxy-5-(pyrrolidin-1-yl)phenyl)cyclopropyl)-2,2′-bipyrimidine;
trans-5-(2-(3,4-difluoro-5-((S)-3-methoxypyrrolidin-1-yl)phenyl)cyclopropyl)-2,2′-bipyrimidine;
trans-(3R)-1-(5-(2-([2,2′-bipyrimidin]-5-yl)cyclopropyl)-2,3-difluorophenyl)pyrrolidin-3-ol;
trans-(3S)-1-(5-(2-([2,2′-bipyrimidin]-5-yl)cyclopropyl)-2,3-difluorophenyl)pyrrolidin-3-ol;
trans-5-(2-(3-chloro-4-fluoro-5-(pyrrolidin-1-yl)phenyl)cyclopropyl)-2,2′-bipyrimidine;
trans-5-(2-(3-chloro-4-fluoro-5-((R)-3-methoxypyrrolidin-1-yl)phenyl)cyclopropyl)-2,2′-bipyrimidine;
trans-5-(2-(3-fluoro-5-methoxyphenyl)cyclopropyl)-2,2′-bipyrimidine;
trans-5-(2-(4-chloro-2,3-dimethoxyphenyl)cyclopropyl)-2,2′-bipyrimidine;
trans-5-(2-(3-chloro-4-fluoro-5-((S)-3-methoxypyrrolidin-1-yl)phenyl)cyclopropyl)-2,2′-bipyrimidine;
trans-5-(2-(3-chloro-5-fluorophenyl)cyclopropyl)-2,2′-bipyrimidine;
trans-(3S)-1-(5-(2-([2,2′-bipyrimidin]-5-yl)cyclopropyl)-3-chloro-2-fluorophenyl)pyrrolidin-3-ol;
trans-5-(2-(3,4-difluoro-5-(3-methoxyazetidin-1-yl)phenyl)cyclopropyl)-2,2′-bipyrimidine;
trans-2-(5-(2-(3,4-difluoro-5-methoxyphenyl)cyclopropyl)pyridin-2-yl)pyrimidine;
trans-(3R)-1-(5-(2-([2,2′-bipyrimidin]-5-yl)cyclopropyl)-3-chloro-2-fluorophenyl)pyrrolidin-3-ol;
trans-5-(2-(3-(cyclopropylmethoxy)-4,5-difluorophenyl)cyclopropyl)-2,2′-bipyrimidine;
trans-5-(2-(3,4-difluoro-5-(3-methoxypropoxy)phenyl)cyclopropyl)-2,2′-bipyrimidine;
trans-5-(2-(2,4-dichloro-3-methoxyphenyl)cyclopropyl)-2,2′-bipyrimidine;
trans-5-(2-(3,4-difluoro-5-(2-methoxyethoxy)phenyl)cyclopropyl)-2,2′-bipyrimidine;
trans-2-(5-(2-([2,2′-bipyrimidin]-5-yl)cyclopropyl)-2,3-difluorophenyl)-7-oxa-2-azaspiro[3.5]nonane;
trans-5-(2-(3-(3,3-dimethylazetidin-1-yl)-4,5-difluorophenyl)cyclopropyl)-2,2′-bipyrimidine;
trans-6-(5-(2-(4-fluoro-3-methoxyphenyl)cyclopropyl)-[2,2′-bipyrimidin]-4-yl)-2-methylbenzo[d]thiazole;
trans-5-(2-(4-Fluoro-3-methoxyphenyl)cyclopropyl)-4-phenyl-2,2′-bipyrimidine;
trans-5-(2-(4-fluoro-3-methoxyphenyl)cyclopropyl)-4-(piperidin-1-yl)-2,2′-bipyrimidine;
trans-5-(2-(4-fluoro-3,5-dimethoxyphenyl)cyclopropyl)-2,2′-bipyrimidine;
trans-5-(2-(3-chloro-4-fluoro-5-methoxyphenyl)cyclopropyl)-2,2′-bipyrimidine;
trans-5-(2-(4-fluoro-3-methoxy-5-methylphenyl)cyclopropyl)-2,2′-bipyrimidine;
trans-5-(2-(3-methoxy-4-(trifluoromethoxy)phenyl)cyclopropyl)-2,2′-bipyrimidine;
trans-5-(2-(3-methoxy-4-(trifluoromethyl)phenyl)cyclopropyl)-2,2′-bipyrimidine;
trans-5-(2-(5-chloro-4-fluoro-2-(3-methoxypropoxy)phenyl)cyclopropyl)-2,2′-bipyrimidine;
trans-5-(2-(5-chloro-4-fluoro-2-(2-methoxyethoxy)phenyl)cyclopropyl)-2,2′-bipyrimidine;
trans-5-(2-(5-chloro-4-methoxy-[1,1′-biphenyl]-2-yl)cyclopropyl)-2,2′-bipyrimidine;
trans-5-(2-(3,4,5-trifluorophenyl)cyclopropyl)-2,2′-bipyrimidine;
trans-5-(2-(3-methoxy-5-(trifluoromethyl)phenyl)cyclopropyl)-2,2′-bipyrimidine;
trans-5-(2-(3-methoxy-5-(trifluoromethoxy)phenyl)cyclopropyl)-2,2′-bipyrimidine;
trans-5-(2-(4-fluoro-3-methoxy-5-(trifluoromethyl)phenyl)cyclopropyl)-2,2′-bipyrimidine;
trans-5-(2-(naphthalen-1-yl)cyclopropyl)-2,2′-bipyrimidine;
trans-5-(2-(naphthalen-2-yl)cyclopropyl)-2,2′-bipyrimidine;
trans-5-(2-(4-fluoronaphthalen-1-yl)cyclopropyl)-2,2′-bipyrimidine;
trans-5-(2-(4-fluoronaphthalen-2-yl)cyclopropyl)-2,2′-bipyrimidine;
trans-3-(2-([2,2′-bipyrimidin]-5-yl)cyclopropyl)imidazo[1,2-a]pyridine;
trans-5-(2-([2,2′-bipyrimidin]-5-yl)cyclopropyl)quinoline;
trans-5-(2-([2,2′-bipyrimidin]-5-yl)cyclopropyl)-8-fluoroquinoline;
trans-5-(2-([2,2′-bipyrimidin]-5-yl)cyclopropyl)-8-methoxyquinoline;
trans-5-(2-(3,4-difluoro-5-methoxyphenyl)cyclopropyl)-2-(pyridin-2-yl)pyrimidine;
trans-5-(2-(4-fluoro-3,5-dimethoxyphenyl)cyclopropyl)-2-(pyridin-2-yl)pyrimidine;
trans-2-(pyridin-2-yl)-5-(2-(3,4,5-trimethoxyphenyl)cyclopropyl)pyrimidine;
trans-5-(2-(3,4-difluoro-5-methoxyphenyl)cyclopropyl)-2,4′-bipyrimidine;
trans-5-(2-(3,4-difluoro-5-methoxyphenyl)cyclopropyl)-2-(pyrazin-2-yl)pyrimidine;
trans-5-(2-(3,4-difluoro-5-methoxyphenyl)cyclopropyl)-2-(3-fluoropyridin-2-yl)pyrimidine;
trans-5-(2-(3,4-difluoro-5-methoxyphenyl)cyclopropyl)-2-(3-methoxypyridin-2-yl)pyrimidine;
trans-5-(2-(3,4-difluoro-5-(1H-imidazol-1-yl)phenyl)cyclopropyl)-2,2′-bipyrimidine;
trans-5-(2-(5,6-dimethoxypyridin-3-yl)cyclopropyl)-2,2′-bipyrimidine;
trans-5-(2-(3-(difluoromethoxy)-4-fluorophenyl)cyclopropyl)-2,2′-bipyrimidine;
trans-5-(2-(4-fluoro-3-(4-methylpiperazin-1-yl)phenyl)cyclopropyl)-2,2′-bipyrimidine;
trans-5-(2-(2,4-difluoro-3-methoxyphenyl)cyclopropyl)-2,2′-bipyrimidine;
trans-3-(2-(4-fluoro-3-methoxyphenyl)cyclopropyl)-6-(pyrimidin-2-yl)pyridazine;
trans-4-(2-(4-fluoro-3-methoxyphenyl)cyclopropyl)-1-(pyrimidin-2-yl)isoquinoline;
trans-5-(2-(4-fluoro-3-(piperidin-1-yl)phenyl)cyclopropyl)-2,2′-bipyrimidine;
trans-1-(5-(2-([2,2′-bipyrimidin]-5-yl)cyclopropyl)-2-fluorophenyl)pyrrolidin-2-one;
trans-5-(2-(4-chloro-3-(pyrrolidin-1-yl)phenyl)cyclopropyl)-2,2′-bipyrimidine;
trans-5-((2-([2,2′-bipyrimidin]-5-yl)cyclopropyl)-2-chloro-N-methylbenzamide;
trans-5-(2-([2,2′-bipyrimidin]-5-yl)cyclopropyl)-2-chloro-N,N-dimethylbenzamide;
trans-N-(5-(2-([2,2′-bipyrimidin]-5-yl)cyclopropyl)-2-chlorophenyl)acetamide;
trans-5-(2-([2,2′-bipyrimidin]-5-yl)cyclopropyl)-N-cyclopentyl-2,3-difluoroaniline;
trans-6-(5-(2-([2,2′-bipyrimidin]-5-yl)cyclopropyl)-2,3-difluorophenyl)-2-oxa-6-azaspiro[3.3]heptane;
trans-5-(2-(3,4-difluoro-5-(3-isopropoxyazetidin-1-yl)phenyl)cyclopropyl)-2,2′-bipyrimidine;
trans-5-(2-(3-(3-(tert-butyl sulfonyl)azetidin-1-yl)-4,5-difluorophenyl)cyclopropyl)-2,2′-bipyrimidine;
trans-5-(2-(3,4-difluoro-5-(3-(2-methoxyethoxy)azetidin-1-yl)phenyl)cyclopropyl)-2,2′-bipyrimidine;
trans-5-(2-(3-(3-(3,4-difluoro-5-methoxyphenyl)azetidin-1-yl)-4,5-difluorophenyl)cyclopropyl)-2,2′-bipyrimidine;
trans-5-(2-(3-(3-(3,4-difluorophenyl)azetidin-1-yl)-4,5-difluorophenyl)cyclopropyl)-2,2′-bipyrimidine;
trans-5-(2-(3,4-difluoro-5-(3-(4-fluoro-3-methoxyphenyl)azetidin-1-yl)phenyl)cyclopropyl)-2,2′-bipyrimidine;
trans-5-(2-(3-(3-(3,4-dimethoxyphenyl)azetidin-1-yl)-4,5-difluorophenyl)cyclopropyl)-2,2′-bipyrimidine;
trans-5-(2-(3,4-difluoro-5-((1-methyl-1H-1,2,4-triazol-3-yl)methoxy)phenyl)cyclopropyl)-2,2′-bipyrimidine;
trans-2-((5-(2-([2,2′-bipyrimidin]-5-yl)cyclopropyl)-2,3-difluorophenoxy)methyl)thiazole;
trans-5-(2-(3,4-difluoro-5-((1-methyl-1H-pyrazol-3-yl)methoxy)phenyl)cyclopropyl)-2,2′-bipyrimidine;
trans-5-(2-(3-(3,3-dimethylpyrrolidin-1-yl)-4,5-difluorophenyl)cyclopropyl)-2,2′-bipyrimidine;
trans-6-(5-(2-([2,2′-bipyrimidin]-5-yl)cyclopropyl)-2,3-difluorophenyl)-2-oxa-6-azaspiro[3.4]octane;
trans-1-((3S)-3-((5-(2-([2,2′-bipyrimidin]-5-yl)cyclopropyl)-2,3-difluorophenyl)amino)pyrrolidin-1-yl)ethenone;
trans-1-((3R)-3-((5-(2-([2,2′-bipyrimidin]-5-yl)cyclopropyl)-2,3-difluorophenyl) amino) pyrrolidin-1-yl)ethenone;
trans-(3S)-N-(5-(2-([2,2′-bipyrimidin]-5-yl)cyclopropyl)-2,3-difluorophenyl)-1-methylpyrrolidin-3-amine;
trans-(3R)-N-(5-(2-([2,2′-bipyrimidin]-5-yl)cyclopropyl)-2,3-difluorophenyl)-1-methylpyrrolidin-3-amine;
trans-5-(2-(3,4-difluoro-5-(4-methyl-1H-pyrazol-1-yl)phenyl)cyclopropyl)-2,2′-bipyrimidine;
trans-N-(5-(2-([2,2′-bipyrimidin]-5-yl)cyclopropyl)-2,3-difluorophenyl)-N-methyloxetan-3-amine;
trans-(1-(5-(2-([2,2′-bipyrimidin]-5-yl)cyclopropyl)-2,3-difluorophenyl)-1H-pyrazol-4-yl)methanol;
trans-5-(2-(343R,4 S)-3,4-dimethoxypyrrolidin-1-yl)-4,5-difluorophenyl)cyclopropyl)-2,2′-bipyrimidine;
trans-5-(2-(3,4-difluoro-5-(4-(methoxymethyl)-1H-pyrazol-1-yl)phenyl)cyclopropyl)-2,2′-bipyrimidine;
trans-1-(5-(2-([2,2′-bipyrimidin]-5-yl)cyclopropyl)-2,3-difluorophenyl)-5,6-dimethoxy-1H-benzo[d]imidazole;
trans-2-(5-(2-([2,2′-bipyrimidin]-5-yl)cyclopropyl)-2,3-difluorophenyl)-2-azaspiro[3.3]heptan-6-ol;
trans-(3R,4R)-1-(5-(2-([2,2′-bipyrimidin]-5-yl)cyclopropyl)-2,3-difluorophenyl)pyrrolidine-3,4-diol;
trans-4-(3-(5-(2-([2,2′-bipyrimidin]-5-yl)cyclopropyl)-2,3-difluorophenoxy)propyl)morpholine;
trans-4-(3-(4-(2-([2,2′-bipyrimidin]-5-yl)cyclopropyl)-2,6-difluorophenoxy)propyl)morpholine;
trans-5-(2-(4((2-oxaspiro[3.3]heptan-6-yl)oxy)-3,5-difluorophenyl)cyclopropyl)-2,2′-bipyrimidine;
trans-5-(2-(3-((2-oxaspiro[3.3]heptan-6-yl)oxy)-4,5-difluorophenyl)cyclopropyl)-2,2′-bipyrimidine;
trans-2-((5-(2-([2,2′-bipyrimidin]-5-yl)cyclopropyl)-2,3-difluorophenyl)(methyl)amino)ethan-1-ol;
trans-5-(2-(3-(cyclopentyloxy)-4,5-difluorophenyl)cyclopropyl)-2,2′-bipyrimidine;
trans-5-(2-(3,4-difluoro-5-((R)-tetrahydrofuran-3-yl)oxy)phenyl)cyclopropyl)-2,2′-bipyrimidine;
trans-5-(2-(3,4-difluoro-5-((S)-tetrahydrofuran-3-yl)oxy)phenyl)cyclopropyl)-2,2′-bipyrimidine;
trans-5-(2-(3,4-difluoro-5-(4-methoxy-1H-pyrazol-1-yl)phenyl)cyclopropyl)-2,2′-bipyrimidine;
trans-5-(2-(3,4-difluoro-5-(1H-pyrazol-1-yl)phenyl)cyclopropyl)-2,2′-bipyrimidine;
trans-5-(2-(3,4-difluoro-5-(3-phenylazetidin-1-yl)phenyl)cyclopropyl)-2,2′-bipyrimidine;
trans-5-(2-(3-(3,4-difluoro-1H-pyrrol-1-yl)-4,5-difluorophenyl)cyclopropyl)-2,2′-bipyrimidine;
trans-1-(5-(2-([2,2′-bipyrimidin]-5-yl)cyclopropyl)-2,3-difluorophenyl)-1H-pyrazol-4-ol;
trans-5-(2-(3,4-difluoro-5-(4-methyl-1H-imidazol-1-yl)phenyl)cyclopropyl)-2,2′-bipyrimidine;
trans-ethyl 2-([2,2′-bipyrimidin]-5-yl)-3-(3,4-difluoro-5-methoxyphenyl)cyclopropane-1-carboxylate;
trans-4-(2-([2,2′-bipyrimidin]-5-yl)cyclopropyl)-6,7-difluoro-1-(3-methoxypropyl)-1H-indazole;
trans-6-(2-([2,2′-bipyrimidin]-5-yl)cyclopropyl)-4-fluoro-2-(3-methoxypropyl)-2H-indazole;
trans-2-(2-([2,2′-bipyrimidin]-5-yl)cyclopropyl)-4,6-difluorobenzo[d]thiazole;
trans-6-(2-([2,2′-bipyrimidin]-5-yl)cyclopropyl)-4-fluoro-1-isopropyl-2-methyl-1H-benzo[d]imidazole;
trans-6-(2-([2,2′-bipyrimidin]-5-yl)cyclopropyl)-4-fluoro-2-methylbenzo[d]thiazole;
trans-5-(2-([2,2′-bipyrimidin]-5-yl)cyclopropyl)-7-fluoro-1-(3-methoxypropyl)-1H-benzo[d]imidazole;
trans-6-(2-([2,2′-bipyrimidin]-5-yl)cyclopropyl)-4-fluoro-1-(3-methoxypropyl)-1H-benzo[d]imidazole;
trans-6-(2-([2,2′-bipyrimidin]-5-yl)cyclopropyl)-4-fluoro-1-(3-methoxypropyl)-1H-indazole;
trans-4-(2-([2,2′-bipyrimidin]-5-yl)cyclopropyl)-7-fluoro-1-(3-methoxypropyl)-1H-indazole;
trans-4-(2-([2,2′-bipyrimidin]-5-yl)cyclopropyl)-7-fluoro-2-(3-methoxypropyl)-2H-indazole; and
trans-5-(2-([2,2′-bipyrimidin]-5-yl)cyclopropyl)-2-methylbenzo[d]thiazole.

Embodiment 22 provides a pharmaceutical composition comprising at least one compound of any of Embodiments 1-21 and at least one pharmaceutically acceptable carrier.

Embodiment 23 provides the pharmaceutical composition of Embodiment 22, further comprising at least one additional agent useful for treating hepatitis infection.

Embodiment 24 provides the pharmaceutical composition of Embodiment 23, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; and GalNAc-siRNA conjugate targeted against an HBV gene transcript.

Embodiment 25 provides the pharmaceutical composition of Embodiment 24, wherein the immunostimulator is a checkpoint inhibitor.

Embodiment 26 provides the pharmaceutical composition of Embodiment 25, wherein the checkpoint inhibitor is a PD-L1 inhibitor.

Embodiment 27 provides the pharmaceutical composition of any of Embodiments 23-26, wherein the hepatitis virus is at least one selected from the group consisting of hepatitis B virus (HBV) and hepatitis D virus (HDV).

Embodiment 28 provides a method of treating, ameliorating, and/or preventing hepatitis virus infection in a subject, the method comprising administering to the subject in need thereof a therapeutically effective amount of the compound of any of Embodiments 1-21 and/or the pharmaceutical composition of any of Embodiments 22-27, or a salt, solvate, stereoisomer, tautomer, or any mixtures thereof.

Embodiment 29 provides the method of Embodiment 28, wherein the subject is infected with hepatitis B virus (HBV).

Embodiment 30 provides the method of any of Embodiments 28-29, wherein the subject is further infected with hepatitis D virus (HDV).

Embodiment 31 provides the method of any of Embodiments 28-30, wherein the subject is infected with HBV and HDV.

Embodiment 32 provides the method of any of Embodiments 28-31, wherein the subject is further administered at least one additional agent useful for treating the hepatitis virus infection.

Embodiment 33 provides the method of Embodiment 32, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; and GalNAc-siRNA conjugate targeted against an HBV gene transcript.

Embodiment 34 provides the method of Embodiment 33, wherein the immunostimulator is a checkpoint inhibitor.

Embodiment 35 provides the method of Embodiment 34, wherein the checkpoint inhibitor is a PD-L1 inhibitor.

Embodiment 36 provides the method of any of Embodiments 32-35, wherein the subject is co-administered the at least one compound and the at least one additional agent.

Embodiment 37 provides the method of any of Embodiments 32-36, wherein the at least one compound and the at least one additional agent are coformulated.

Embodiment 38 provides the method of any of Embodiments 28-37, wherein the subject is a mammal.

Embodiment 39 provides the method of Embodiment 38, wherein the mammal is human.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A compound of formula (I) or (II) or (III), or a salt, solvate, geometric isomer, stereoisomer, tautomer, and any mixtures thereof: 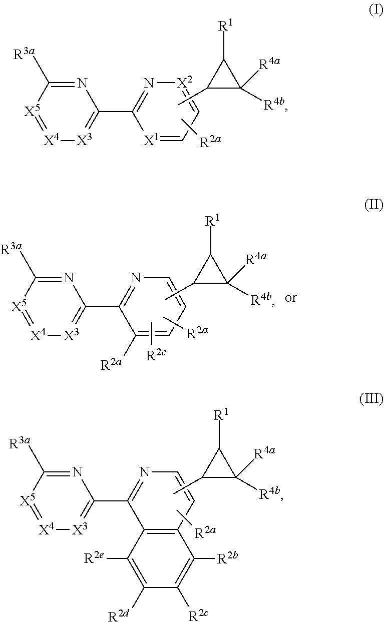
wherein:
one of the following applies:
(i) X¹ is N, X² is CR^{2b}, or
(ii) X¹ is CR^{2c}, X² is N;
one of the following applies:
(i) X³ is N, X⁴ is CR^{3c}, X⁵ is CR^{3d}; or
(ii) X³ is CR^{3b}, X⁴ is N, X⁵ is CR^{3d}; or
(iii) X³ is CR^{3b}, X⁴ is CR^{3c}, X⁵ is N;
R¹ is selected from the group consisting of optionally substituted phenyl, optionally substituted naphthyl, optionally substituted pyridinyl, optionally substituted pyrimidinyl, optionally substituted benzo[d]thiazolyl; optionally substituted benzoimidazolyl, optionally substituted imidazo[1,2-a]pyridinyl, optionally substituted quinolinyl, optionally substituted isoquinolinyl, optionally substituted 1H-indazolyl, and optionally substituted 2H-indazolyl;
each occurrence of R^{2a}, R^{2b}, R^{2c}, R^{2d}, and R^{2e} is independently selected from the group consisting of H, halogen, cyano, nitro, optionally substituted C₁-C₆ alkyl, optionally substituted C₁-C₆ haloalkyl, optionally substituted C₃-C₈ cycloalkyl, optionally substituted C₁-C₆ haloalkoxy, optionally substituted C₁-C₆ hydroxyalkyl, —OR′, -SR′, —S(═O)R′, —S(O)₂R′, —N(R′)(R′), —N(R′)C(═O)(R′), —C(═O)N(R′)(R′), optionally substituted phenyl, optionally substituted heterocyclyl, and optionally substituted heteroaryl,
wherein each occurrence of R′ is independently selected from the group consisting of H, optionally substituted C₁-C₆ alkyl, and optionally substituted C₃-C₈ cycloalkyl;
each occurrence of R^{3a}, R^{3b}, R^{3c}, and R^{3d} is independently selected from the group consisting of H, halogen, cyano, nitro, optionally substituted C₁-C₆ alkyl, optionally substituted C₁-C₆ haloalkyl, optionally substituted C₃-C₈ cycloalkyl, optionally substituted C₁-C₆ haloalkoxy, optionally substituted C₁-C₆ hydroxyalkyl, —OR″, —SR″, —S(═O)R″, —S(O)₂R″, —N(R″)(R″), optionally substituted phenyl, optionally substituted heterocyclyl, and optionally substituted heteroaryl,
wherein each occurrence of R″ is independently selected from the group consisting of H, optionally substituted C₁-C₆ alkyl, and optionally substituted C₃-C₈ cycloalkyl;
R^{4a} is selected from the group consisting of R^{4c}, F, Cl, Br, I, —OR^{4c}, and —C(═O)OR^{4c}, wherein each occurrence of R^{4c} is independently H, optionally substituted C₁-C₆ alkyl, or optionally substituted C₃-C₈ cycloalkyl, and
R^{4b} is selected from the group consisting of R^{4d}, F, Cl, Br, I, —OR^{4d}, and —C(═O)OR^{4d}, wherein each occurrence of R^{4d} is independently H, optionally substituted C₁-C₆ alkyl, or optionally substituted C₃-C₈ cycloalkyl.

2. The compound of claim 1, wherein in (I) X¹ is N and X² is CR^{2b}.

3. The compound of claim 1, wherein R¹ is substituted with at least one selected from the group consisting of H, F, Cl, Br, I, optionally substituted C₁-C₆ alkyl, optionally substituted C₁-C₆ haloalkyl, optionally substituted C₁-C₆ alkoxy, optionally substituted C₁-C₆ haloalkoxy, optionally substituted phenyl, —NR″′Ir, —C(═O)NR″′Ir, —NHC(═O)R″′, and optionally substituted heterocyclyl; wherein each occurrence of R″′ is independently selected from the group consisting of H, optionally substituted C₁-C₆ alkyl, optionally substituted C₃-C₈ cycloalkyl, and optionally substituted heterocyclyl.

4. The compound of claim 1, wherein R¹ is substituted with at least one selected from the group consisting of: H; F; Cl; Br; I; C₁-C₆ alkyl; C₁-C₆ alkyl substituted with at least one of F, Cl, Br, I, OH, CN, C₁-C₆ alkyl, C₃-C₈ cycloalkyl, C₁-C₆ alkoxy, and C₃-C₈ cycloalkoxy; C₁-C₆ alkoxy; C₁-C₆ alkoxy substituted with at least one of F, Cl, Br, I, OH, CN, C₁-C₆ alkyl, C₃-C₈ cycloalkyl, C₁-C₆ alkoxy, and C₃-C₈ cycloalkoxy; C₃-C₈ cycloalkyl; C₃-C₈ cycloalkyl substituted with at least one of F, Cl, Br, I, OH, CN, C₁-C₆ alkyl, C₃-C₈ cycloalkyl, C₁-C₆ alkoxy, and C₃-C₈ cycloalkoxy; C₃-C₈ cycloalkoxy; C₃-C₈ cycloalkoxy substituted with at least one of F, Cl, Br, I, OH, CN, C₁-C₆ alkyl, C₃-C₈ cycloalkyl, C₁-C₆ alkoxy, and C₃-C₈ cycloalkoxy; —NH₂; —NH(C₁-C₆ alkyl); —N(C₁-C₆ alkyl)(C₁-C₆ alkyl); —C(═O)NH(C₁-C₆ alkyl); —NHC(═O)H; —NHC(═O)(C₁-C₆ alkyl); optionally substituted phenyl; optionally substituted spiroheterocyclyl; optionally substituted pyrrolidinonyl; optionally substituted azetidinyl; optionally substituted pyrrolidinyl; optionally substituted pyrrolidinonyl; optionally substituted piperidinyl; optionally substituted piperazinyl; optionally substituted morpholinyl; optionally substituted pyrrolyl; optionally substituted pyrazolyl; optionally substituted imidazolyl; optionally substituted 1H-benzo[d]imidazyl; optionally substituted indazolyl; optionally substituted benzo[d]thiazolyl; and optionally substituted 1H-benzo[d]imidazolyl.

5. The compound of claim 1, wherein R¹ is substituted with at least one selected from the group consisting of: H, F, Cl, Br, I, methyl, difluoromethyl, trifluoromethyl, ethyl, propyl, isopropyl, phenyl, methoxy, ethoxy, propoxy, isopropoxy, 2-methoxyethoxy, 3-methoxypropoxy, cyclopropylmethoxy, 2,2-difluoroethoxy, difluoromethoxy, trifluoromethoxy, (1-methyl-1H-1,2,4-triazol-3-yl)methoxy, (thiazol-2-yl)methoxy, (1-methyl-1H-pyrazol-3-yl)methoxy, 3-N-morpholinyl-propoxy, tetrahydrofuranoxy, dimethylamino, diethylamino, N-2-hydroxyethylamino, N-methyl-N-2-hydroxyethylamino, cyclopropylamino, cyclobutylamino, cyclopentylamino, cyclohexylamino, N-methylpyrrolidinyl-methylamino, N-acetylpyrrolidinyl-methylamino, (N-methyl)(N-methyl-oxet-3-yl)amino, dimethylamino, pyrrolidin-2-on-1-yl, azetidinyl, 3,3-dimethylazetidinyl, 3-hydroxy-azetidinyl, 3-methoxy-azetidinyl, 3-ethoxy-azetidinyl, 3-propoxy-azetidinyl, 3-isopropoxy-azetidinyl, 3-(2-methoxyethoxy)azetidinyl, 3-(tert-butylsulfonyl)azetidinyl, 3-(optionally substituted phenyl)azetidinyl, pyrrolidinyl, pyrrolidin-2-one-1-yl, 3-hydroxypyrrolidinyl, 3-methoxypyrrolidinyl, 3,3-difluoropyrrolidinyl, 3,4-dihydroxypyrrolidin-1-yl, 3,4-dimethoxypyrrolidin-1-yl, piperidinyl, piperazinyl, morpholinyl, 1H-pyrrol-1-yl, 3,4-difluoro-1H-pyrrol-1-yl, pyrazol-1-yl, 4-methyl-1H-pyrazol-1-yl, 4-methoxy-1H-pyrazol-1-yl, 4-hydroxymethyl-1H-pyrazol-1-yl, 4-methoxymethyl-1H-pyrazol-1-yl, 1H-imidazol-1-yl, methyl-1H-imidazol-1-yl, N4-(C₁-C₆ alkyl)-piperazin-1-yl, N4-[SO₂(C₁-C₆ alkyl)]-piperazin-1-yl, imidazolyl, —C(═O)NH₂, —C(═O)NHCH₃, —C(═O)N(CH₃)₂, —NHC(═O)CH_{3, 2}-oxa-6-azaspiro[3.3]hept-6-yl, 2-oxa-6-azaspiro[3.3]hept-6-oxy, 2-azaspiro[3.3]hept-2-yl, 6-hydroxy-2-azaspiro[3.3]hept-2-yl, 2-oxaspiro[3.3]hept-6-yl, 2-oxa-6-azaspiro[3.4]oct-6-yl, 7-oxa-2-azaspiro[3.5]non-2-yl, 5,6-dimethoxy-1H-benzo[d]imidazyl, 1H-indazolyl, 6,7-difluoro-1-(3-methoxypropyl)-1H-indazolyl, 4-fluoro-2-(3-methoxypropyl)-2H-indazolyl, 7-fluoro-1-(3-methoxypropyl)-1H-indazolyl, 7-fluoro-2-(3-methoxypropyl)-2H-indazolyl, 4-fluoro-1-(3-methoxypropyl)-1H-indazolyl, benzo[d]thiazolyl, 2-methylbenzo[d]thiazolyl, 4,6-difluorobenzo[d]thiazolyl, 4-fluoro-2-methylbenzo[d]thiazolyl, 1H-benzo[d]imidazolyl, 4-fluoro-1-isopropyl-2-methyl-1H-benzo [d]imidazolyl, 7-fluoro-1-(3-methoxypropyl)-1H-benzo[d]imidazolyl, and 4-fluoro-1-(3-methoxypropyl)-1H-benzo[d]imidazolyl.

6. The compound of claim 1, wherein R¹ is selected from the group consisting of:

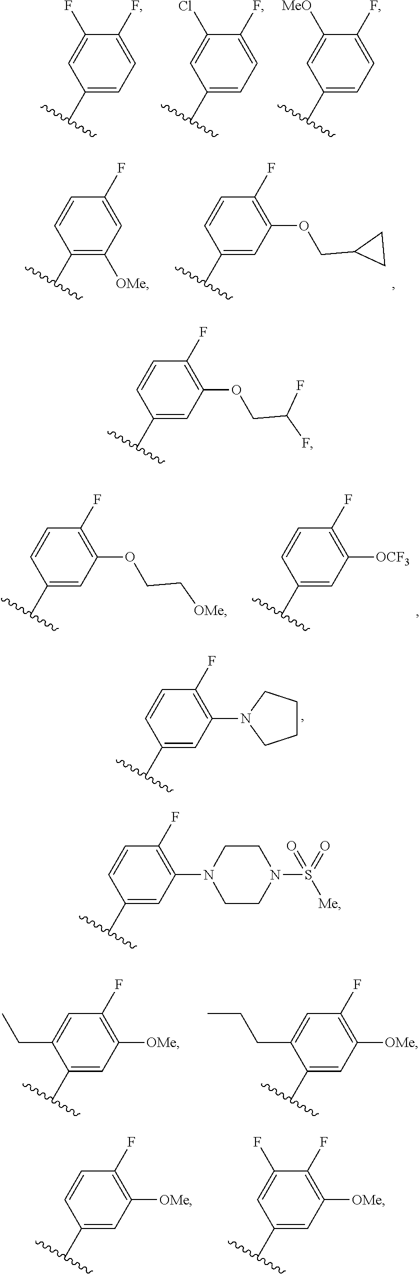

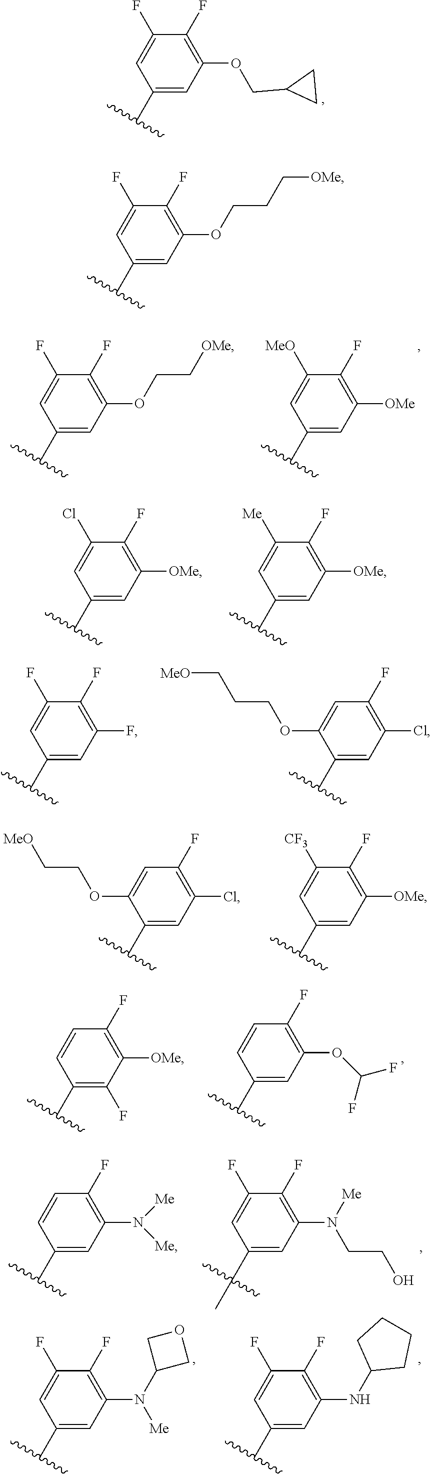

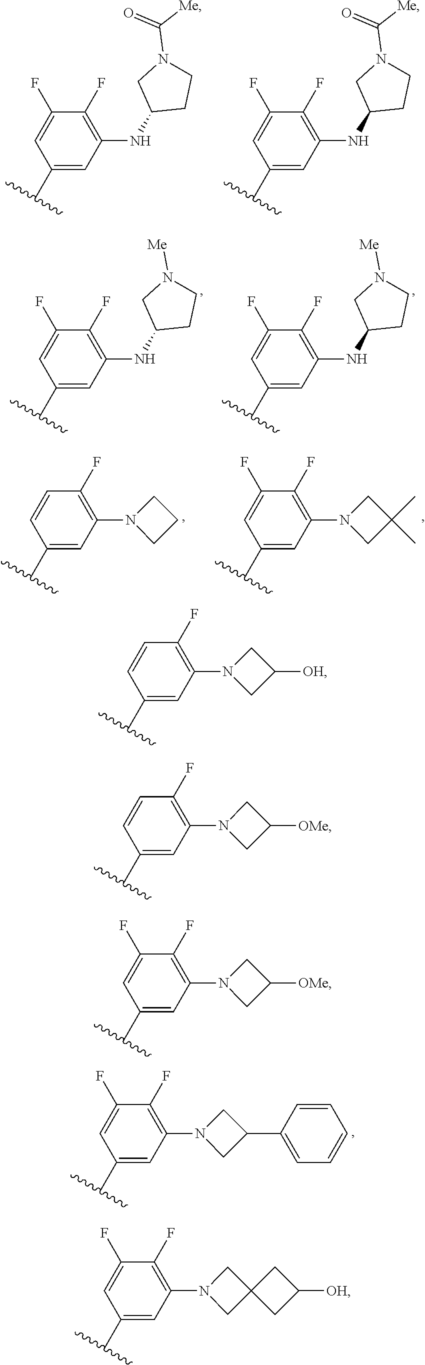

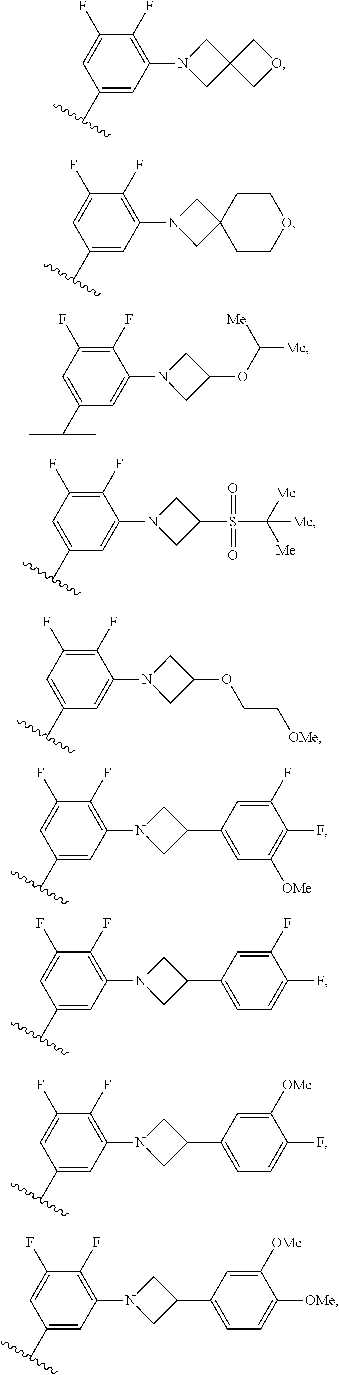

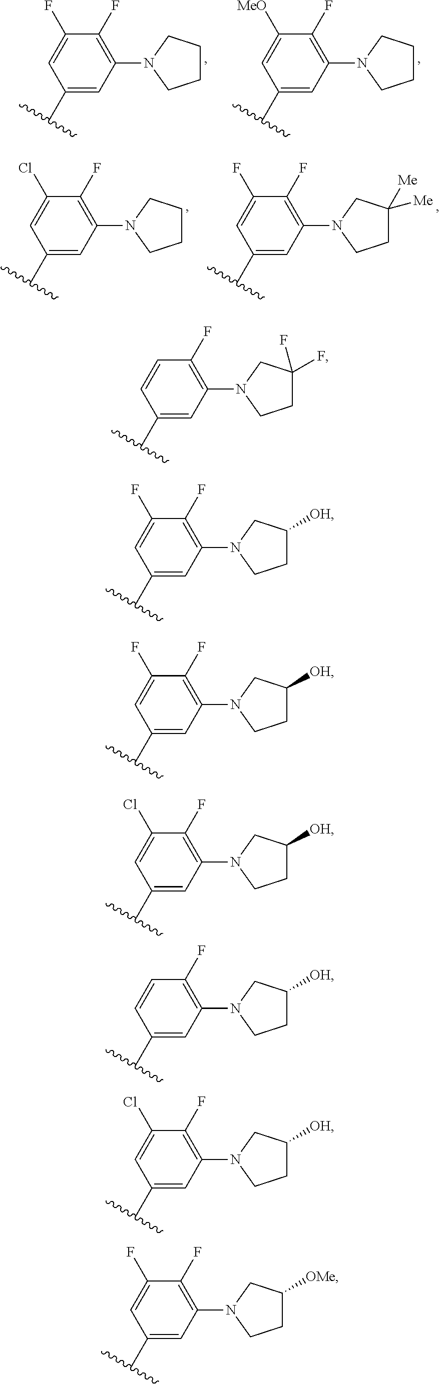

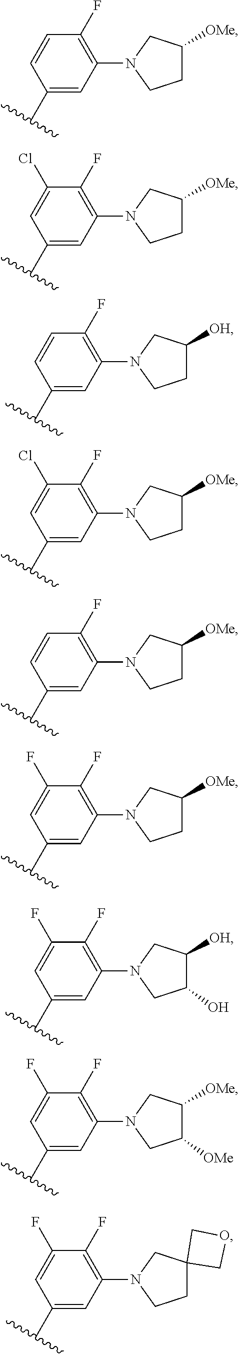

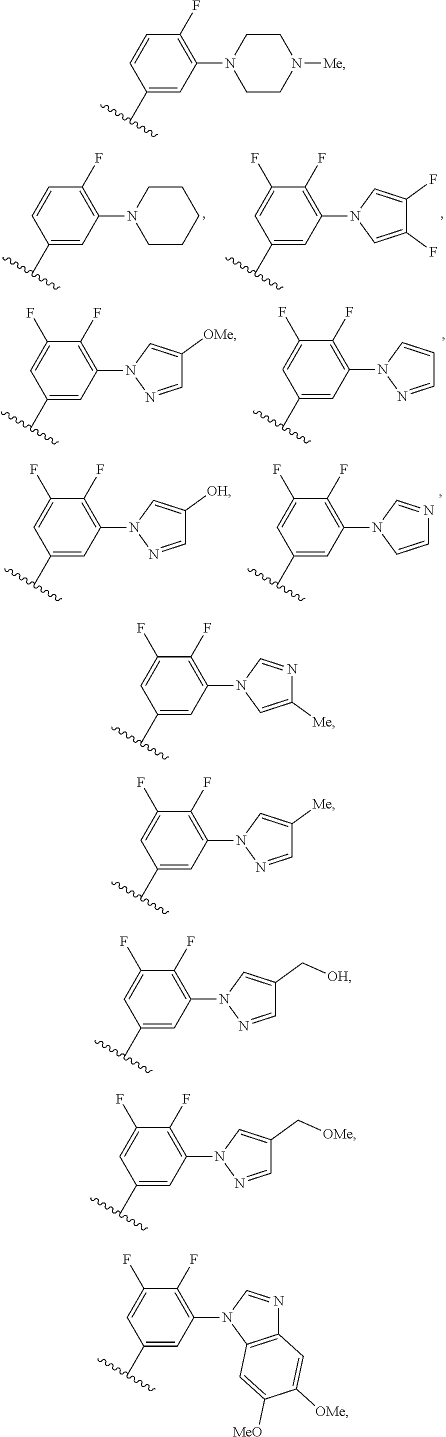

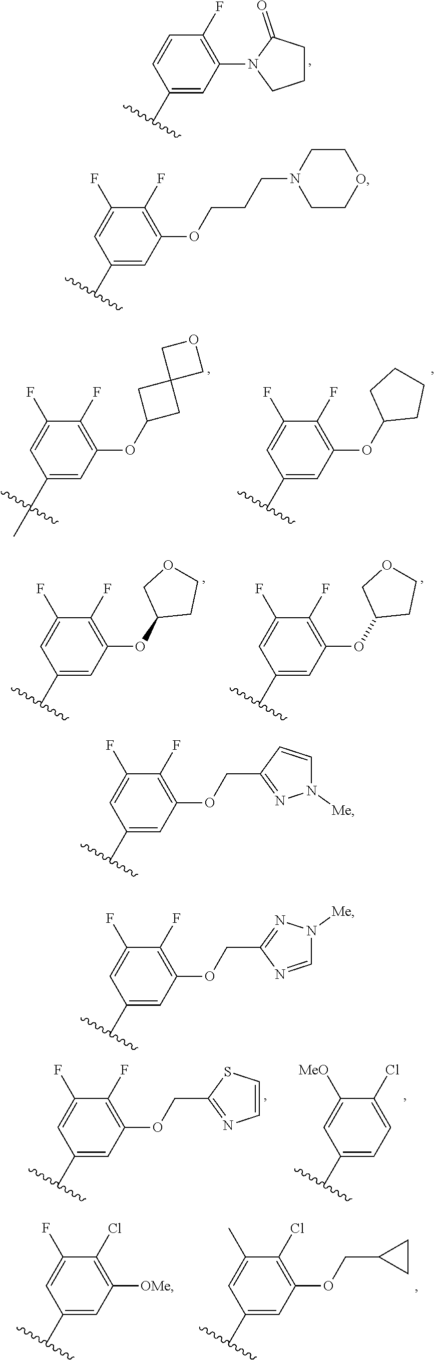

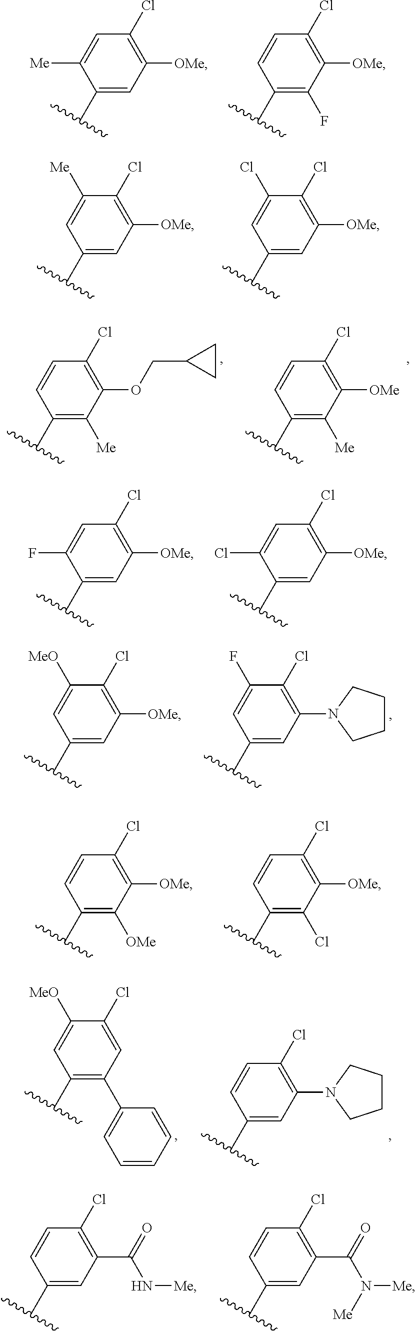

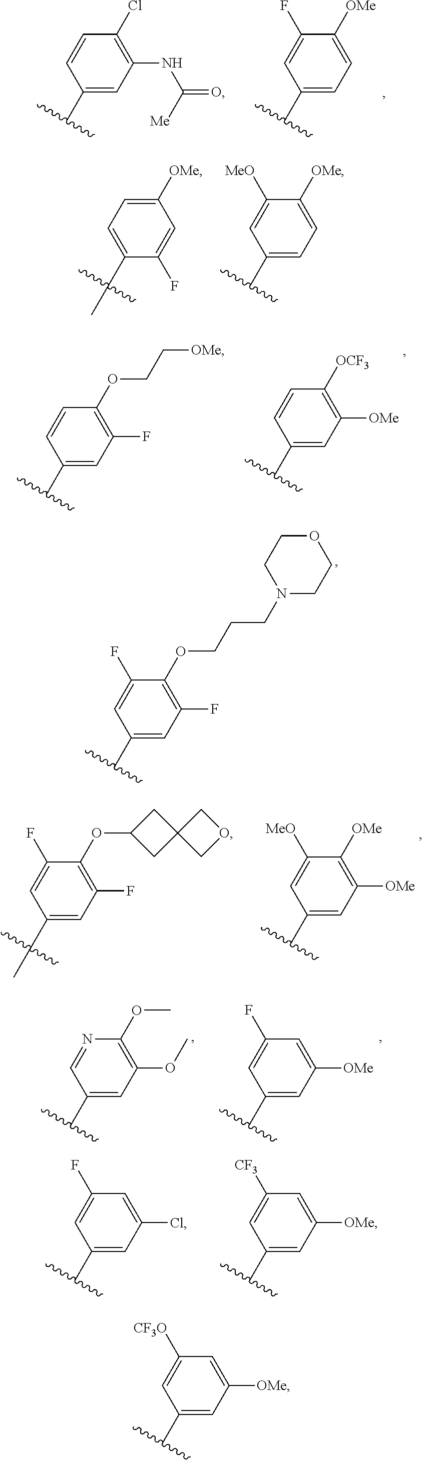

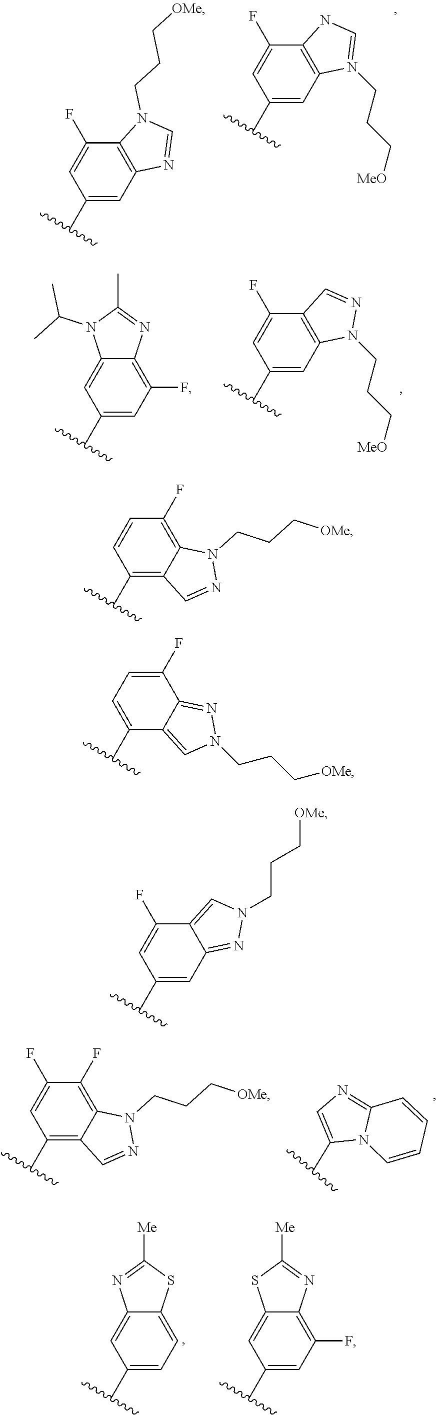

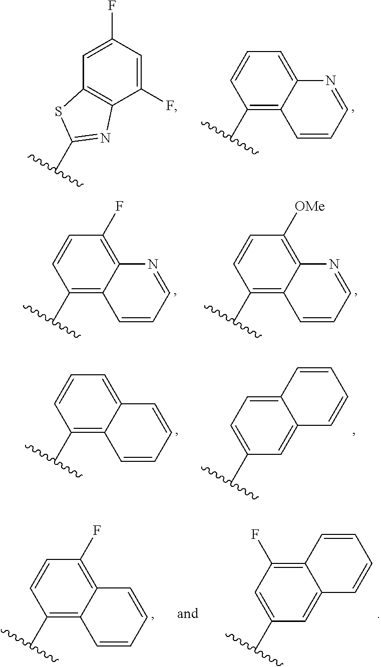

7. The compound of claim 1, wherein one of the following applies:
(a) each occurrence of R^{2a}, R^{2b}, R^{2c}, R^{2d}, and R^{2e} is independently selected from the group consisting of H, F, Cl, Br, I, methyl, ethyl, isopropyl, cyclopropyl, cyclobutyl, cyclopentyl, cyclohexyl, methoxy, ethoxy, isopropoxy, phenyl, optionally substituted benzo[d]thiazolyl, azetidinyl, pyrrolidinyl, piperidinyl, and piperazinyl; or
(b) each occurrence of R^{2a}, R^{2b}, R^{2c}, R^{2d}, and R^{2e} is independently selected from the group consisting of H, methyl, ethyl, isopropyl, cyclopropyl, cyclobutyl, cyclopentyl, cyclohexyl, methoxy, ethoxy, isopropoxy, phenyl, optionally substituted benzo[d]thiazolyl, azetidinyl, pyrrolidinyl, piperidinyl, and piperazinyl.

8. (canceled)

9. The compound of claim 1, wherein each occurrence of R^{3a}, R^{3b}, R^{3c}, and R^{3d} is independently selected from the group consisting of H, halogen, cyano, nitro, optionally substituted C₁-C₆ alkyl, C₁-C₆ haloalkyl, optionally substituted C₃-C₈ cycloalkyl, C₁-C₆ haloalkoxy, C₁-C₆ hydroxyalkyl, and —OR″′, wherein each occurrence of R″′ is independently selected from the group consisting of H, optionally substituted C₁-C₆ alkyl, and optionally substituted C₃-C₈ cycloalkyl.

10. The compound of claim 1, wherein each occurrence of R^{3b} is independently H, methyl, ethyl, propyl, cyclopropyl, isopropyl, methoxy, ethoxy, propoxy, cyclopropoxy, isopropoxy, fluoro, chloro, bromo, or iodo.

11. The compound of claim 1, wherein each occurrence of alkyl, alkylenyl (alkylene), cycloalkyl, heterocyclyl, or carbocyclyl is independently optionally substituted with at least one substituent selected from the group consisting of C₁-C₆ alkyl, halogen, —OR″′, phenyl (thus yielding, in non-limiting examples, optionally substituted phenyl-(C₁-C₃ alkyl), —S(O)₂R″′, and —N(R″′)(R″′), wherein each occurrence of R″′ is independently H, optionally substituted C₁-C₆ alkyl, or optionally substituted C₃-C₈ cycloalkyl.

12. The compound of claim 1, wherein one of the following applies:
(a) each occurrence of aryl or heteroaryl is independently optionally substituted with at least one substituent selected from the group consisting of C₁-C₆ alkyl, C₁-C₆ haloalkyl, C₁-C₆ haloalkoxy, halogen, —CN, —OR″″, —N(R″″)(R″″), —NO₂, S(O)₂R″″, —S(═O)₂N(R″″)(R″″), acyl, and C₁-C₆ alkoxycarbonyl, wherein each occurrence of R″″ is independently H, optionally substituted C₁-C₆ alkyl, or optionally substituted C₃-C₈ cycloalkyl, or
(b) each occurrence of aryl or heteroaryl is independently optionally substituted with at least one substituent selected from the group consisting of C₁-C₆ alkyl, C₁-C₆ haloalkyl, C₁-C₆ haloalkoxy, halogen, —CN, —OR″″, —N(R″″)(R″″), and C₁-C₆ alkoxycarbonyl, wherein each occurrence of R″″ is independently H, optionally substituted C₁-C₆ alkyl, or optionally substituted C₃-C₈ cycloalkyl.

13. (canceled)

14. The compound of claim 1, wherein one of the following applies:
(a) the compound is selected from the group consisting of: 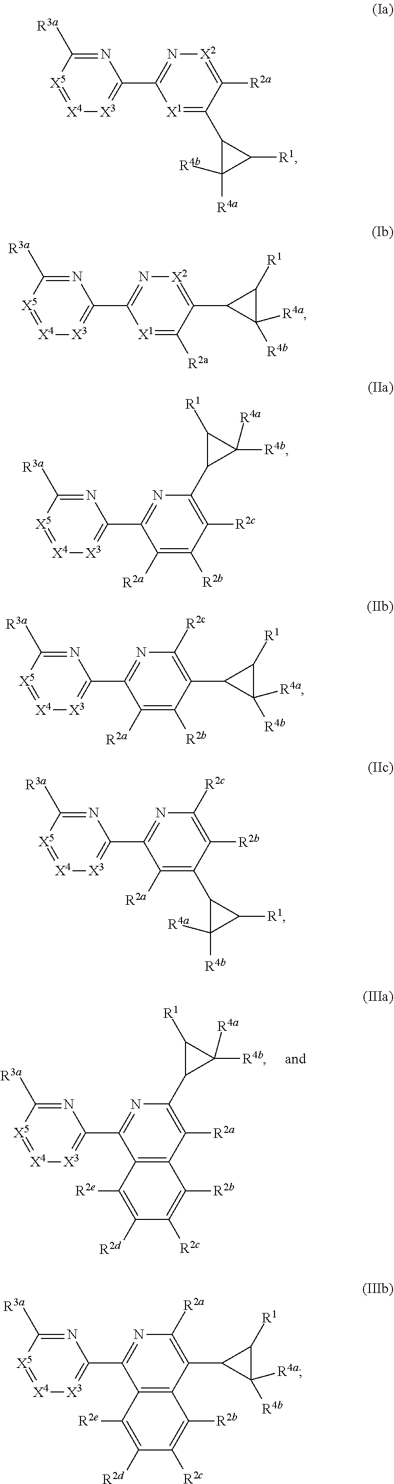
(b) the compound is selected from the group consisting of: 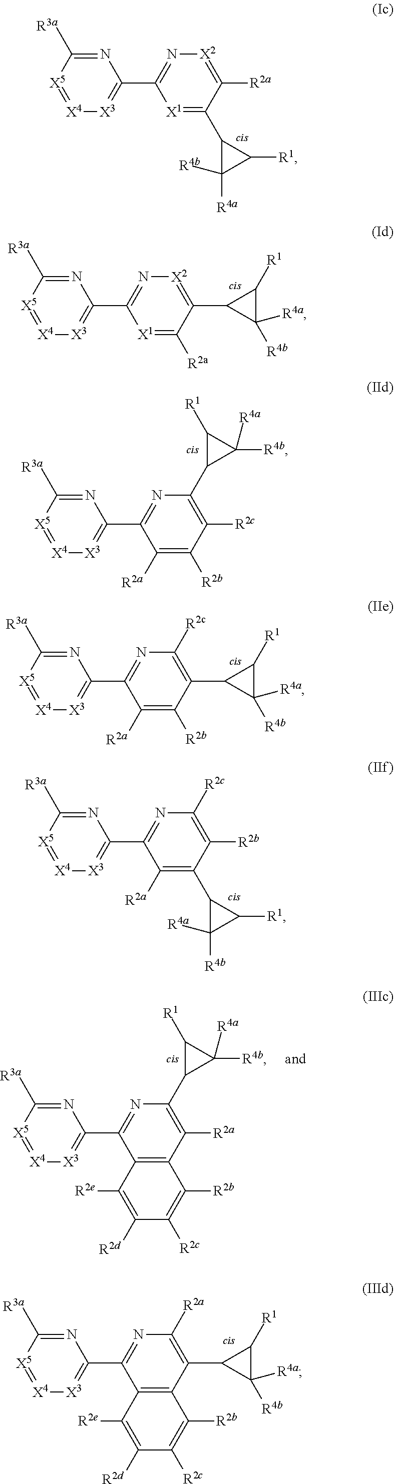
or
(c) the compound is selected from the group consisting of:

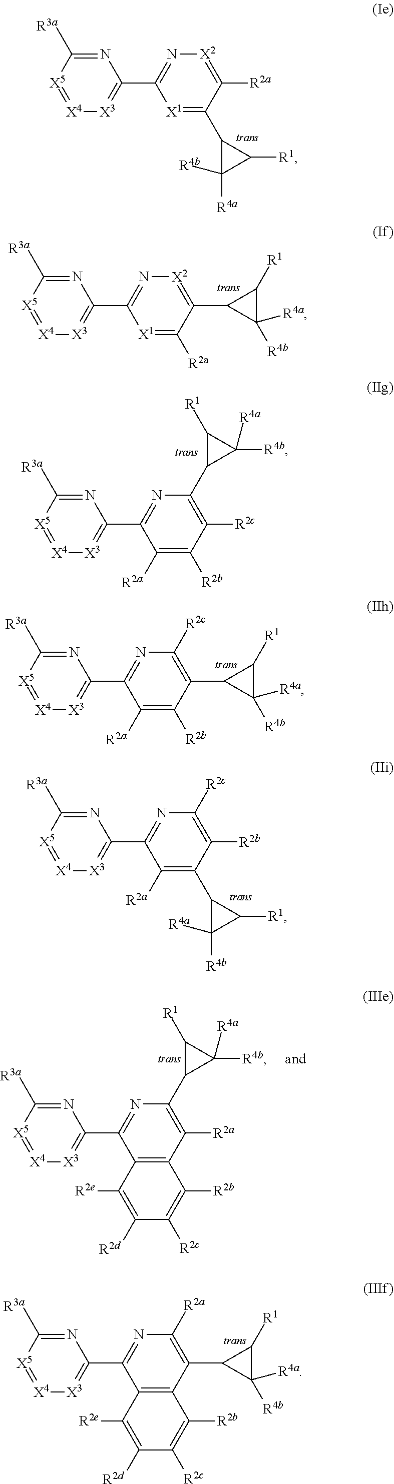

15. (canceled)

16. (canceled)

17. The compound of claim 1, wherein one of the following applies:
(a) the compound is selected from the group consisting of: 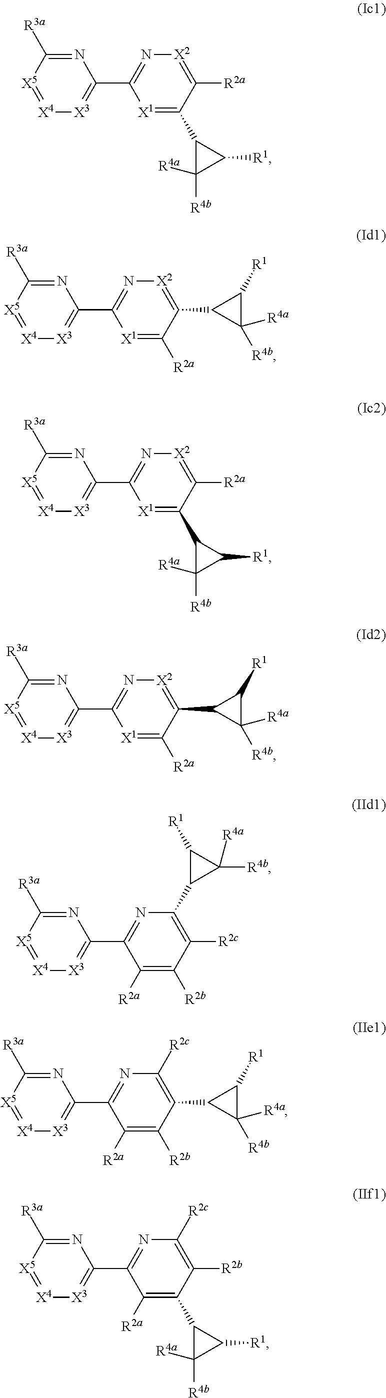 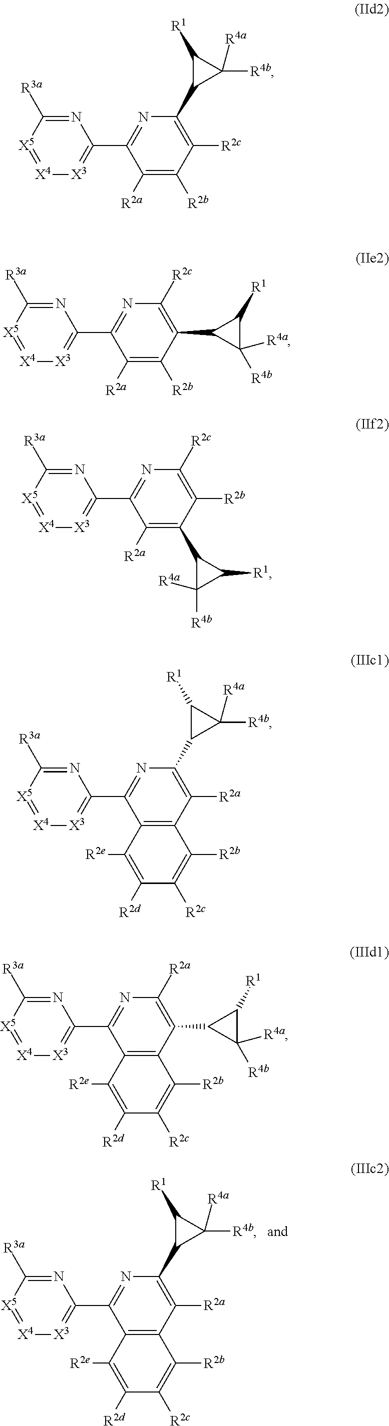 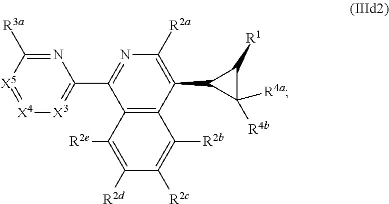
or
(b) the compound is selected from the group consisting of:

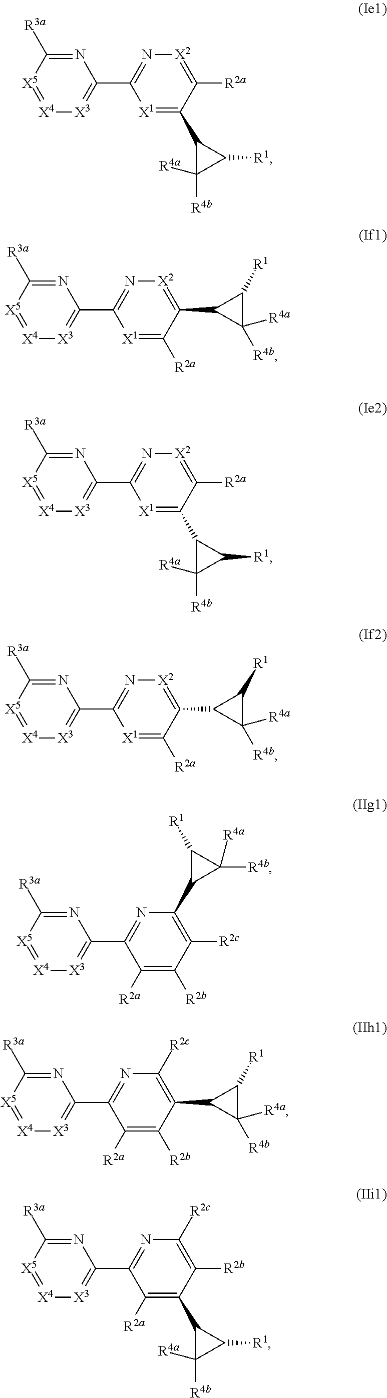

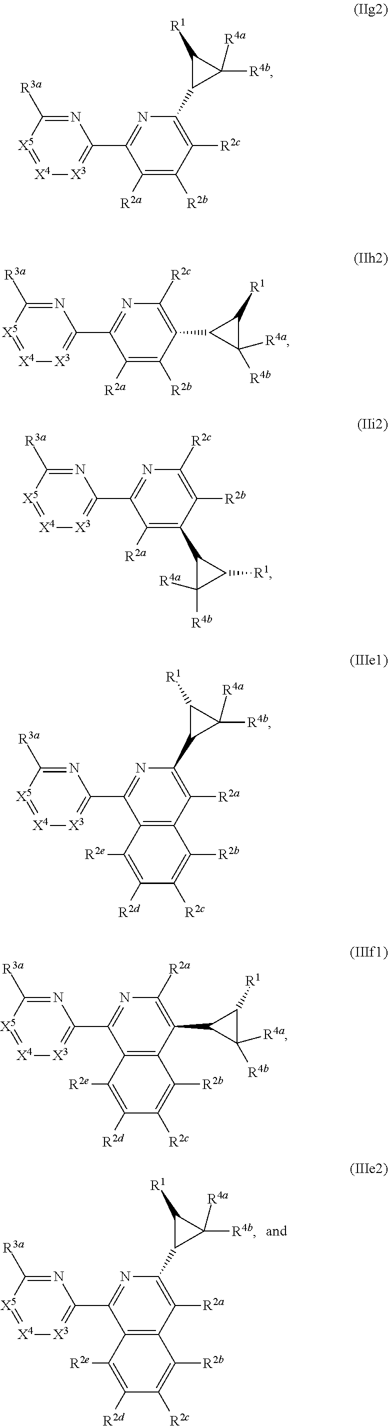

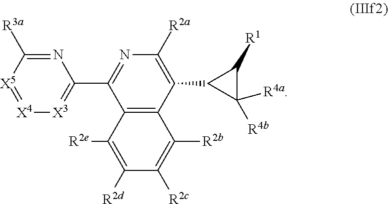

18. (canceled)

19. The compound of claim 1, which is selected from the group consisting of:

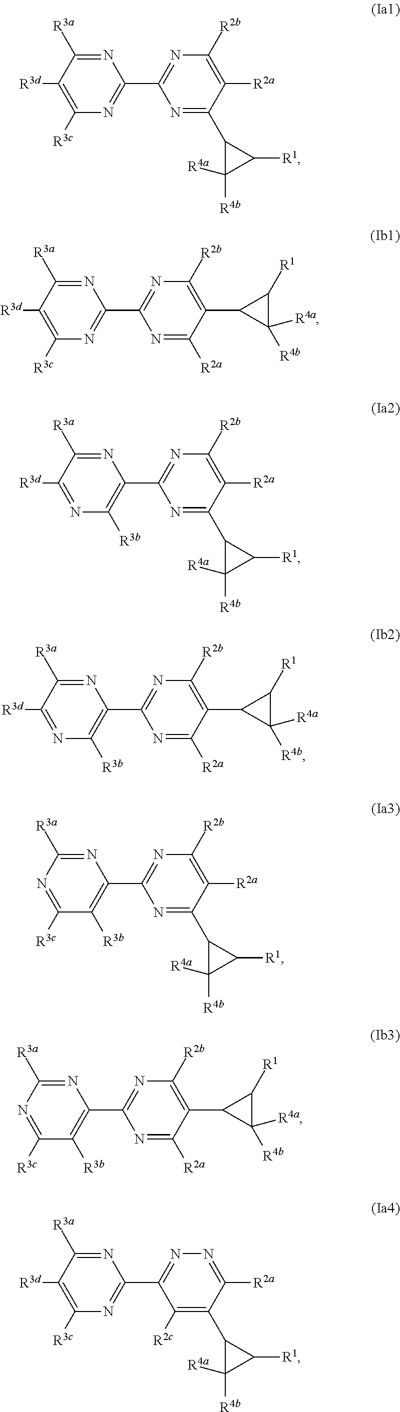

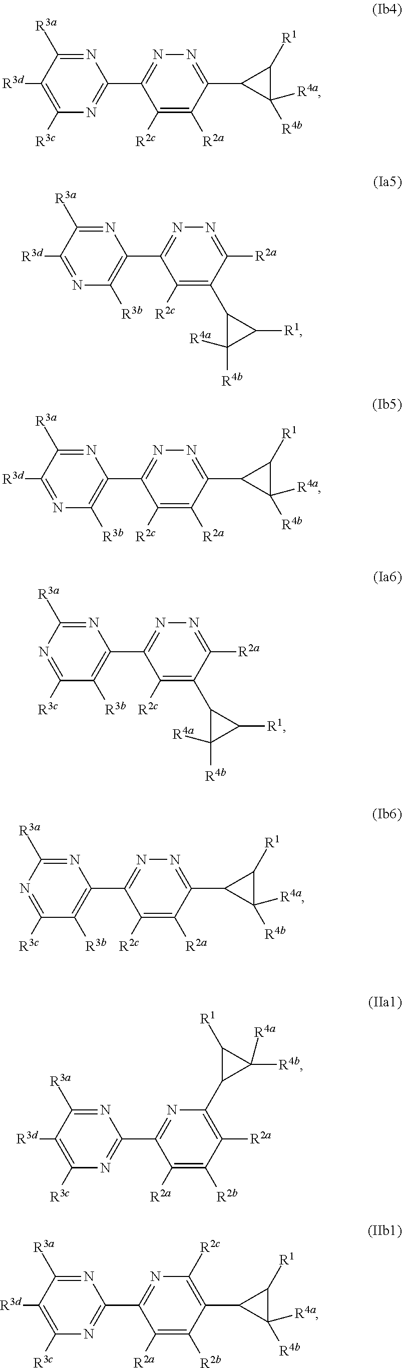

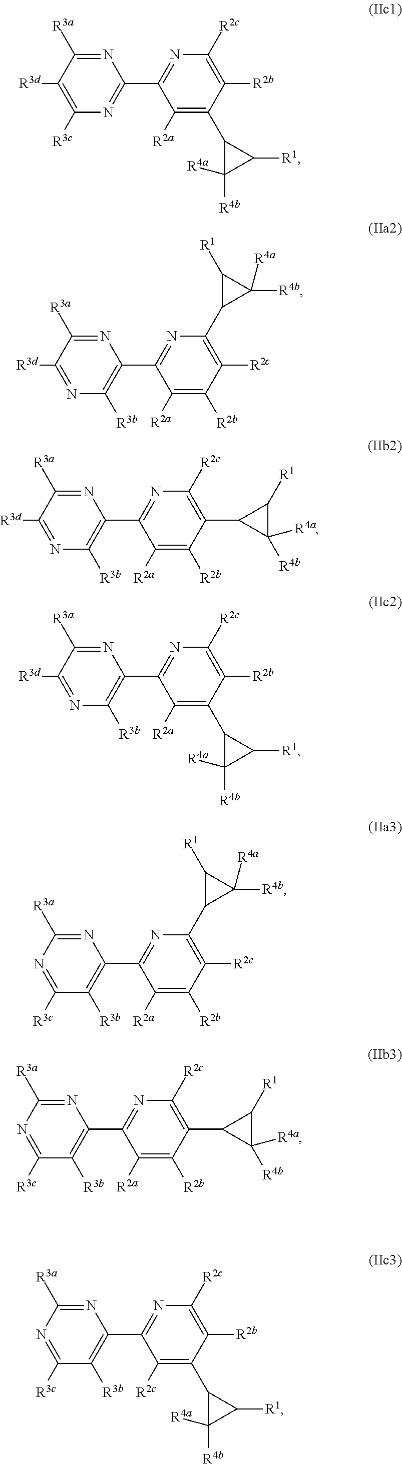

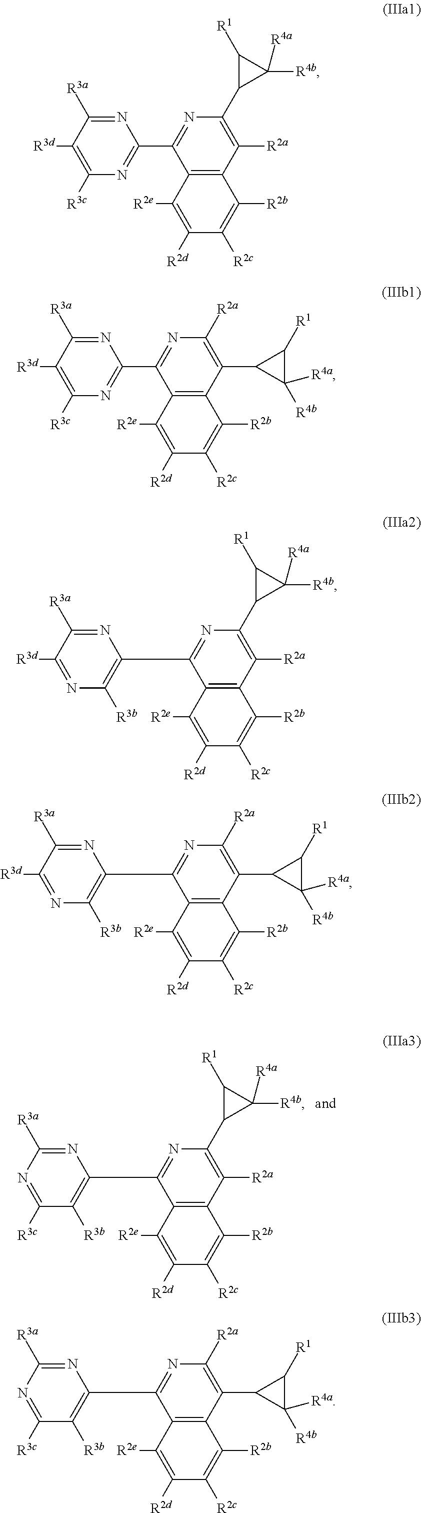

20. The compound of claim 1, which is selected from the group consisting of:
5-(2-(2-fluoro-4-methoxyphenyl)cyclopropyl)-2,2′-bipyrimidine;
4-(2-(3,4-difluorophenyl)cyclopropyl)-2,2′-bipyrimidine;
5-(2-(4-fluoro-2-methoxyphenyl)cyclopropyl)-2,2′-bipyrimidine;
5-(2-(4-fluoro-3-methoxyphenyl)cyclopropyl)-2,2′-bipyrimidine;
5-(2-(3-fluoro-4-methoxyphenyl)cyclopropyl)-2,2′-bipyrimidine;
5-(2-(3,4-difluorophenyl)cyclopropyl)-2,2′-bipyrimidine;
5-(2-(4-fluoro-3-methoxyphenyl)cyclopropyl)-4-methyl-2,2′-bipyrimidine;
5-(2-(4-fluoro-3-(pyrrolidin-1-yl)phenyl)cyclopropyl)-2,2′-bipyrimidine;
5-(2-(4-fluoro-3-(2-methoxyethoxy)phenyl)cyclopropyl)-2,2′-bipyrimidine;
5-(2-(3-fluoro-4-(2-methoxyethoxy)phenyl)cyclopropyl)-2,2′-bipyrimidine;
5-(2-(3-(cyclopropylmethoxy)-4-fluorophenyl)cyclopropyl)-2,2′-bipyrimidine;
5-(2-(3-(2,2-difluoroethoxy)-4-fluorophenyl)cyclopropyl)-2,2′-bipyrimidine;
5-(2-(3-chloro-4-fluorophenyl)cyclopropyl)-2,2′-bipyrimidine;
5-(2-(3,4-dimethoxyphenyl)cyclopropyl)-2,2′-bipyrimidine;
5-(2-(4-chloro-3-methoxyphenyl)cyclopropyl)-2,2′-bipyrimidine;
5-(2-(2-ethyl-4-fluoro-5-methoxyphenyl)cyclopropyl)-2,2′-bipyrimidine;
5-(2-(4-fluoro-5-methoxy-2-propylphenyl)cyclopropyl)-2,2′-bipyrimidine;
5-(2-(4-fluoro-3-(trifluoromethoxy)phenyl)cyclopropyl)-2,2′-bipyrimidine;
5-(2-(4-fluoro-3-(4-(methylsulfonyl)piperazin-1-yl)phenyl)cyclopropyl)-2,2′-bipyrimidine;
5-(2-(3-(3,3-difluoropyrrolidin-1-yl)-4-fluorophenyl)cyclopropyl)-2,2′-bipyrimidine;
5-(2-(4-chloro-3-fluoro-5-methoxyphenyl)cyclopropyl)-2,2′-bipyrimidine;
5-(2-(4-fluoro-3-(3-methoxyazetidin-1-yl)phenyl)cyclopropyl)-2,2′-bipyrimidine;
5-((2-(4-chloro-3-(cyclopropylmethoxy)-5-methylphenyl)cyclopropyl)-2,2′-bipyrimidine;
5-(2-(4-chloro-5-methoxy-2-methylphenyl)cyclopropyl)-2,2′-bipyrimidine;
1-(5-(2-([2,2′-bipyrimidin]-5-yl)cyclopropyl)-2-fluorophenyl)azetidin-3-ol;
5-(2-(4-chloro-2-fluoro-3-methoxyphenyl)cyclopropyl)-2,2′-bipyrimidine;
5-(2-(4-chloro-3-methoxy-5-methylphenyl)cyclopropyl)-2,2′-bipyrimidine;
5-(2-(3,4-dichloro-5-methoxyphenyl)cyclopropyl)-2,2′-bipyrimidine;
5-(2-(4-chloro-3-(cyclopropylmethoxy)-2-methylphenyl)cyclopropyl)-2,2′-bipyrimidine;
5-(2-([2,2′-bipyrimidin]-5-yl)cyclopropyl)-2-fluoro-N,N-dimethylaniline;
(3S)-1-(5-(2-([2,2′-bipyrimidin]-5-yl)cyclopropyl)-2-fluorophenyl)pyrrolidin-3-ol;
5-(2-(4-fluoro-34(S)-3-methoxypyrrolidin-1-yl)phenyl)cyclopropyl)-2,2′-bipyrimidine;
(3R)-1-(5-(2-([2,2′-bipyrimidin]-5-yl)cyclopropyl)-2-fluorophenyl)pyrrolidin-3-ol;
5-(2-(4-chloro-3-methoxy-2-methylphenyl)cyclopropyl)-2,2′-bipyrimidine;
5-(2-(4-fluoro-3-methoxyphenyl)cyclopropyl)-4-isopropyl-2,2′-bipyrimidine;
4-ethyl-5-(2-(4-fluoro-3-methoxyphenyl)cyclopropyl)-2,2′-bipyrimidine;
4-cyclohexyl-5-(2-(4-fluoro-3-methoxyphenyl)cyclopropyl)-2,2′-bipyrimidine;
5-(2-(4-fluoro-3-methoxyphenyl)cyclopropyl)-4-methoxy-2,2′-bipyrimidine;
5-(2-(3-(azetidin-1-yl)-4-fluorophenyl)cyclopropyl)-2,2′-bipyrimidine;
5-(2-(3,4-difluoro-5-methoxyphenyl)cyclopropyl)-2,2′-bipyrimidine;
5-(2-(4-chloro-2-fluoro-5-methoxyphenyl)cyclopropyl)-2,2′-bipyrimidine;
5-(2-(2,4-dichloro-5-methoxyphenyl)cyclopropyl)-2,2′-bipyrimidine;
5-(2-(4-chloro-3-fluoro-5-(pyrrolidin-1-yl)phenyl)cyclopropyl)-2,2′-bipyrimidine;
5-(2-(3,4-difluoro-5-(pyrrolidin-1-yl)phenyl)cyclopropyl)-2,2′-bipyrimidine;
5-(2-(4-fluoro-34(R)-3-methoxypyrrolidin-1-yl)phenyl)cyclopropyl)-2,2′-bipyrimidine;
5-(2-(3,4-difluoro-54R)-3-methoxypyrrolidin-1-yl)phenyl)cyclopropyl)-2,2′-bipyrimidine;
5-(2-(4-chloro-3,5-dimethoxyphenyl)cyclopropyl)-2,2′-bipyrimidine;
5-(2-(4-fluoro-3-methoxy-5-(pyrrolidin-1-yl)phenyl)cyclopropyl)-2,2′-bipyrimidine;
5-(2-(3,4-difluoro-5-((S)-3-methoxypyrrolidin-1-yl)phenyl)cyclopropyl)-2,2′-bipyrimidine;
(3R)-1-(5-(2-([2,2′-bipyrimidin]-5-yl)cyclopropyl)-2,3-difluorophenyl)pyrrolidin-3-ol;
(3S)-1-(5-(2-([2,2′-bipyrimidin]-5-yl)cyclopropyl)-2,3-difluorophenyl)pyrrolidin-3-ol;
5-(2-(3-chloro-4-fluoro-5-(pyrrolidin-1-yl)phenyl)cyclopropyl)-2,2′-bipyrimidine;
5-(2-(3-chloro-4-fluoro-5-((R)-3-methoxypyrrolidin-1-yl)phenyl)cyclopropyl)-2,2′-bipyrimidine;
5-(2-(3-fluoro-5-methoxyphenyl)cyclopropyl)-2,2′-bipyrimidine;
5-(2-(4-chloro-2,3-dimethoxyphenyl)cyclopropyl)-2,2′-bipyrimidine;
5-(2-(3-chloro-4-fluoro-5-((S)-3-methoxypyrrolidin-1-yl)phenyl)cyclopropyl)-2,2′-bipyrimidine;
5-(2-(3-chloro-5-fluorophenyl)cyclopropyl)-2,2′-bipyrimidine;
(3S)-1-(5-(2-([2,2′-bipyrimidin]-5-yl)cyclopropyl)-3-chloro-2-fluorophenyl)pyrrolidin-3-ol;
5-(2-(3,4-difluoro-5-(3-methoxyazetidin-1-yl)phenyl)cyclopropyl)-2,2′-bipyrimidine;
2-(5-(2-(3,4-difluoro-5-methoxyphenyl)cyclopropyl)pyridin-2-yl)pyrimidine;
(3R)-1-(5-(2-([2,2′-bipyrimidin]-5-yl)cyclopropyl)-3-chloro-2-fluorophenyl)pyrrolidin-3-ol;
5-(2-(3-(cyclopropylmethoxy)-4,5-difluorophenyl)cyclopropyl)-2,2′-bipyrimidine;
5-(2-(3,4-difluoro-5-(3-methoxypropoxy)phenyl)cyclopropyl)-2,2′-bipyrimidine;
5-(2-(2,4-dichloro-3-methoxyphenyl)cyclopropyl)-2,2′-bipyrimidine;
5-(2-(3,4-difluoro-5-(2-methoxyethoxy)phenyl)cyclopropyl)-2,2′-bipyrimidine;
2-(5-(2-([2,2′-bipyrimidin]-5-yl)cyclopropyl)-2,3-difluorophenyl)-7-oxa-2-azaspiro[3.5]nonane;
5-(2-(3-(3,3-dimethylazetidin-1-yl)-4,5-difluorophenyl)cyclopropyl)-2,2′-bipyrimidine;
6-(5-(2-(4-fluoro-3-methoxyphenyl)cyclopropyl)-[2,2′-bipyrimidin]-4-yl)-2-methylbenzo[d]thiazole;
5-(2-(4-Fluoro-3-methoxyphenyl)cyclopropyl)-4-phenyl-2,2′-bipyrimidine;
5-(2-(4-fluoro-3-methoxyphenyl)cyclopropyl)-4-(piperidin-1-yl)-2,2′-bipyrimidine;
5-(2-(4-fluoro-3,5-dimethoxyphenyl)cyclopropyl)-2,2′-bipyrimidine;
5-(2-(3-chloro-4-fluoro-5-methoxyphenyl)cyclopropyl)-2,2′-bipyrimidine;
5-(2-(4-fluoro-3-methoxy-5-methylphenyl)cyclopropyl)-2,2′-bipyrimidine;
5-(2-(3-methoxy-4-(trifluoromethoxy)phenyl)cyclopropyl)-2,2′-bipyrimidine;
5-(2-(3-methoxy-4-(trifluoromethyl)phenyl)cyclopropyl)-2,2′-bipyrimidine;
5-(2-(5-chloro-4-fluoro-2-(3-methoxypropoxy)phenyl)cyclopropyl)-2,2′-bipyrimidine;
5-(2-(5-chloro-4-fluoro-2-(2-methoxyethoxy)phenyl)cyclopropyl)-2,2′-bipyrimidine;
5-(2-(5-chloro-4-methoxy-[1,1′-biphenyl]-2-yl)cyclopropyl)-2,2′-bipyrimidine;
5-(2-(3,4,5-trifluorophenyl)cyclopropyl)-2,2′-bipyrimidine;
5-(2-(3-methoxy-5-(trifluoromethyl)phenyl)cyclopropyl)-2,2′-bipyrimidine;
5-(2-(3-methoxy-5-(trifluoromethoxy)phenyl)cyclopropyl)-2,2′-bipyrimidine;
5-(2-(4-fluoro-3-methoxy-5-(trifluoromethyl)phenyl)cyclopropyl)-2,2′-bipyrimidine;
5-(2-(naphthalen-1-yl)cyclopropyl)-2,2′-bipyrimidine;
5-(2-(naphthalen-2-yl)cyclopropyl)-2,2′-bipyrimidine;
5-(2-(4-fluoronaphthalen-1-yl)cyclopropyl)-2,2′-bipyrimidine;
5-(2-(4-fluoronaphthalen-2-yl)cyclopropyl)-2,2′-bipyrimidine;
3-(2-([2,2′-bipyrimidin]-5-yl)cyclopropyl)imidazo[1,2-a]pyridine;
5-(2-([2,2′-bipyrimidin]-5-yl)cyclopropyl)quinoline;
5-(2-([2,2′-bipyrimidin]-5-yl)cyclopropyl)-8-fluoroquinoline;
5-(2-([2,2′-bipyrimidin]-5-yl)cyclopropyl)-8-methoxyquinoline;
5-(2-(3,4-difluoro-5-methoxyphenyl)cyclopropyl)-2-(pyridin-2-yl)pyrimidine;
5-(2-(4-fluoro-3,5-dimethoxyphenyl)cyclopropyl)-2-(pyridin-2-yl)pyrimidine;
2-(pyridin-2-yl)-5-(2-(3,4,5-trimethoxyphenyl)cyclopropyl)pyrimidine;
5-(2-(3,4-difluoro-5-methoxyphenyl)cyclopropyl)-2,4′-bipyrimidine;
5-(2-(3,4-difluoro-5-methoxyphenyl)cyclopropyl)-2-(pyrazin-2-yl)pyrimidine;
5-(2-(3,4-difluoro-5-methoxyphenyl)cyclopropyl)-2-(3-fluoropyridin-2-yl)pyrimidine;
5-(2-(3,4-difluoro-5-methoxyphenyl)cyclopropyl)-2-(3-methoxypyridin-2-yl)pyrimidine;
5-(2-(3,4-difluoro-5-(1H-imidazol-1-yl)phenyl)cyclopropyl)-2,2′-bipyrimidine;
5-(2-(5,6-dimethoxypyridin-3-yl)cyclopropyl)-2,2′-bipyrimidine;
5-(2-(3-(difluoromethoxy)-4-fluorophenyl)cyclopropyl)-2,2′-bipyrimidine;
5-(2-(4-fluoro-3-(4-methylpiperazin-1-yl)phenyl)cyclopropyl)-2,2′-bipyrimidine;
5-(2-(2,4-difluoro-3-methoxyphenyl)cyclopropyl)-2,2′-bipyrimidine;
3-(2-(4-fluoro-3-methoxyphenyl)cyclopropyl)-6-(pyrimidin-2-yl)pyridazine;
4-(2-(4-fluoro-3-methoxyphenyl)cyclopropyl)-1-(pyrimidin-2-yl)isoquinoline;
5-(2-(4-fluoro-3-(piperidin-1-yl)phenyl)cyclopropyl)-2,2′-bipyrimidine;
1-(5-(2-([2,2′-bipyrimidin]-5-yl)cyclopropyl)-2-fluorophenyl)pyrrolidin-2-one;
5-(2-(4-chloro-3-(pyrrolidin-1-yl)phenyl)cyclopropyl)-2,2′-bipyrimidine;
5-((2-([2,2′-bipyrimidin]-5-yl)cyclopropyl)-2-chloro-N-methylbenzamide;
5-(2-([2,2′-bipyrimidin]-5-yl)cyclopropyl)-2-chloro-N,N-dimethylbenzamide;
N-(5-(2-([2,2′-bipyrimidin]-5-yl)cyclopropyl)-2-chlorophenyl)acetamide;
5-(2-([2,2′-bipyrimidin]-5-yl)cyclopropyl)-N-cyclopentyl-2,3-difluoroaniline;
6-(5-(2-([2,2′-bipyrimidin]-5-yl)cyclopropyl)-2,3-difluorophenyl)-2-oxa-6-azaspiro[3.3]heptane;
5-(2-(3,4-difluoro-5-(3-isopropoxyazetidin-1-yl)phenyl)cyclopropyl)-2,2′-bipyrimidine;
5-(2-(3-(3-(tert-butylsulfonyl)azetidin-1-yl)-4,5-difluorophenyl)cyclopropyl)-2,2′-bipyrimidine;
5-(2-(3,4-difluoro-5-(3-(2-methoxyethoxy)azetidin-1-yl)phenyl)cyclopropyl)-2,2′-bipyrimidine;
5-(2-(3-(3-(3,4-difluoro-5-methoxyphenyl)azetidin-1-yl)-4,5-difluorophenyl)cyclopropyl)-2,2′-bipyrimidine;
5-(2-(3-(3-(3,4-difluorophenyl)azetidin-1-yl)-4,5-difluorophenyl)cyclopropyl)-2,2′-bipyrimidine;
5-(2-(3,4-difluoro-5-(3-(4-fluoro-3-methoxyphenyl)azetidin-1-yl)phenyl)cyclopropyl)-2,2′-bipyrimidine;
5-(2-(3-(3-(3,4-dimethoxyphenyl)azetidin-1-yl)-4,5-difluorophenyl)cyclopropyl)-2,2′-bipyrimidine;
5-(2-(3,4-difluoro-5-((1-methyl-1H-1,2,4-triazol-3-yl)methoxy)phenyl)cyclopropyl)-2,2′-bipyrimidine;
2-((5-(2-([2,2′-bipyrimidin]-5-yl)cyclopropyl)-2,3-difluorophenoxy)methyl)thiazole;
5-(2-(3,4-difluoro-5-((1-methyl-1H-pyrazol-3-yl)methoxy)phenyl)cyclopropyl)-2,2′-bipyrimidine;
5-(2-(3-(3,3-dimethylpyrrolidin-1-yl)-4,5-difluorophenyl)cyclopropyl)-2,2′-bipyrimidine;
6-(5-(2-([2,2′-bipyrimidin]-5-yl)cyclopropyl)-2,3-difluorophenyl)-2-oxa-6-azaspiro[3.4]octane;
1-((3S)-3-((5-(2-([2,2′-bipyrimidin]-5-yl)cyclopropyl)-2,3-difluorophenyl)amino)pyrrolidin-1-yl)ethenone;
1-((3R)-3-((5-(2-([2,2′-bipyrimidin]-5-yl)cyclopropyl)-2,3-difluorophenyl) amino) pyrrolidin-1-yl)ethenone;
(3S)-N-(5-(2-([2,2′-bipyrimidin]-5-yl)cyclopropyl)-2,3-difluorophenyl)-1-methylpyrrolidin-3-amine;
(3R)-N-(5-(2-([2,2′-bipyrimidin]-5-yl)cyclopropyl)-2,3-difluorophenyl)-1-methylpyrrolidin-3-amine;
5-(2-(3,4-difluoro-5-(4-methyl-1H-pyrazol-1-yl)phenyl)cyclopropyl)-2,2′-bipyrimidine;
N-(5-(2-([2,2′-bipyrimidin]-5-yl)cyclopropyl)-2,3-difluorophenyl)-N-methyloxetan-3-amine;
(1-(5-(2-([2,2′-bipyrimidin]-5-yl)cyclopropyl)-2,3-difluorophenyl)-1H-pyrazol-4-yl)methanol;
5-(2-(3-((3R,4S)-3,4-dimethoxypyrrolidin-1-yl)-4,5-difluorophenyl)cyclopropyl)-2,2′-bipyrimidine;
5-(2-(3,4-difluoro-5-(4-(methoxymethyl)-1H-pyrazol-1-yl)phenyl)cyclopropyl)-2,2′-bipyrimidine;
1-(5-(2-([2,2′-bipyrimidin]-5-yl)cyclopropyl)-2,3-difluorophenyl)-5,6-dimethoxy-1H-benzo[d]imidazole;
2-(5-(2-([2,2′-bipyrimidin]-5-yl)cyclopropyl)-2,3-difluorophenyl)-2-azaspiro[3.3]heptan-6-ol;
(3R,4R)-1-(5-(2-([2,2′-bipyrimidin]-5-yl)cyclopropyl)-2,3-difluorophenyl)pyrrolidine-3,4-diol;
4-(3-(5-(2-([2,2′-bipyrimidin]-5-yl)cyclopropyl)-2,3-difluorophenoxy)propyl)morpholine;
4-(3-(4-(2-([2,2′-bipyrimidin]-5-yl)cyclopropyl)-2,6-difluorophenoxy)propyl)morpholine;
5-(2-(4-((2-oxaspiro[3.3]heptan-6-yl)oxy)-3,5-difluorophenyl)cyclopropyl)-2,2′-bipyrimidine;
5-(2-(3-((2-oxaspiro[3.3]heptan-6-yl)oxy)-4,5-difluorophenyl)cyclopropyl)-2,2′-bipyrimidine;
2-((5-(2-([2,2′-bipyrimidin]-5-yl)cyclopropyl)-2,3-difluorophenyl)(methyl)amino)ethan-1-ol;
5-(2-(3-(cyclopentyloxy)-4,5-difluorophenyl)cyclopropyl)-2,2′-bipyrimidine;
5-(2-(3,4-difluoro-5-(((R)-tetrahydrofuran-3-yl)oxy)phenyl)cyclopropyl)-2,2′-bipyrimidine;
5-(2-(3,4-difluoro-5-(((S)-tetrahydrofuran-3-yl)oxy)phenyl)cyclopropyl)-2,2′-bipyrimidine;
5-(2-(3,4-difluoro-5-(4-methoxy-1H-pyrazol-1-yl)phenyl)cyclopropyl)-2,2′-bipyrimidine;
5-(2-(3,4-difluoro-5-(1H-pyrazol-1-yl)phenyl)cyclopropyl)-2,2′-bipyrimidine;
5-(2-(3,4-difluoro-5-(3-phenylazetidin-1-yl)phenyl)cyclopropyl)-2,2′-bipyrimidine;
5-(2-(3-(3,4-difluoro-1H-pyrrol-1-yl)-4,5-difluorophenyl)cyclopropyl)-2,2′-bipyrimidine;
1-(5-(2-([2,2′-bipyrimidin]-5-yl)cyclopropyl)-2,3-difluorophenyl)-1H-pyrazol-4-ol;
5-(2-(3,4-difluoro-5-(4-methyl-1H-imidazol-1-yl)phenyl)cyclopropyl)-2,2′-bipyrimidine;
ethyl 2-([2,2′-bipyrimidin]-5-yl)-3-(3,4-difluoro-5-methoxyphenyl)cyclopropane-1-carboxylate;
4-(2-([2,2′-bipyrimidin]-5-yl)cyclopropyl)-6,7-difluoro-1-(3-methoxypropyl)-1H-indazole;
6-(2-([2,2′-bipyrimidin]-5-yl)cyclopropyl)-4-fluoro-2-(3-methoxypropyl)-2H-indazole;
2-(2-([2,2′-bipyrimidin]-5-yl)cyclopropyl)-4,6-difluorobenzo[d]thiazole;
6-(2-([2,2′-bipyrimidin]-5-yl)cyclopropyl)-4-fluoro-1-isopropyl-2-methyl-1H-benzo[d]imidazole;
6-(2-([2,2′-bipyrimidin]-5-yl)cyclopropyl)-4-fluoro-2-methylbenzo[d]thiazole;
5-(2-([2,2′-bipyrimidin]-5-yl)cyclopropyl)-7-fluoro-1-(3-methoxypropyl)-1H-benzo[d]imidazole;
6-(2-([2,2′-bipyrimidin]-5-yl)cyclopropyl)-4-fluoro-1-(3-methoxypropyl)-1H-benzo[d]imidazole;
6-(2-([2,2′-bipyrimidin]-5-yl)cyclopropyl)-4-fluoro-1-(3-methoxypropyl)-1H-indazole;
4-(2-([2,2′-bipyrimidin]-5-yl)cyclopropyl)-7-fluoro-1-(3-methoxypropyl)-1H-indazole;
4-(2-([2,2′-bipyrimidin]-5-yl)cyclopropyl)-7-fluoro-2-(3-methoxypropyl)-2H-indazole; and
5-(2-([2,2′-bipyrimidin]-5-yl)cyclopropyl)-2-methylbenzo[d]thiazole.

21. The compound of claim 1, which is selected from the group consisting of:
trans-5-(2-(2-fluoro-4-methoxyphenyl)cyclopropyl)-2,2′-bipyrimidine;
trans-4-(2-(3,4-difluorophenyl)cyclopropyl)-2,2′-bipyrimidine;
trans-5-(2-(4-fluoro-2-methoxyphenyl)cyclopropyl)-2,2′-bipyrimidine;
trans-5-(2-(4-fluoro-3-methoxyphenyl)cyclopropyl)-2,2′-bipyrimidine;
trans-5-(2-(3-fluoro-4-methoxyphenyl)cyclopropyl)-2,2′-bipyrimidine;
trans-5-(2-(3,4-difluorophenyl)cyclopropyl)-2,2′-bipyrimidine;
trans-5-(2-(4-fluoro-3-methoxyphenyl)cyclopropyl)-4-methyl-2,2′-bipyrimidine;
trans-5-(2-(4-fluoro-3-(pyrrolidin-1-yl)phenyl)cyclopropyl)-2,2′-bipyrimidine;
trans-5-(2-(4-fluoro-3-(2-methoxyethoxy)phenyl)cyclopropyl)-2,2′-bipyrimidine;
trans-5-(2-(3-fluoro-4-(2-methoxyethoxy)phenyl)cyclopropyl)-2,2′-bipyrimidine;
trans-5-(2-(3-(cyclopropylmethoxy)-4-fluorophenyl)cyclopropyl)-2,2′-bipyrimidine;
trans-5-(2-(3-(2,2-difluoroethoxy)-4-fluorophenyl)cyclopropyl)-2,2′-bipyrimidine;
trans-5-(2-(3-chloro-4-fluorophenyl)cyclopropyl)-2,2′-bipyrimidine;
trans-5-(2-(3,4-dimethoxyphenyl)cyclopropyl)-2,2′-bipyrimidine;
trans-5-(2-(4-chloro-3-methoxyphenyl)cyclopropyl)-2,2′-bipyrimidine;
trans-5-(2-(2-ethyl-4-fluoro-5-methoxyphenyl)cyclopropyl)-2,2′-bipyrimidine;
trans-5-(2-(4-fluoro-5-methoxy-2-propylphenyl)cyclopropyl)-2,2′-bipyrimidine;
trans-5-(2-(4-fluoro-3-(trifluoromethoxy)phenyl)cyclopropyl)-2,2′-bipyrimidine;
trans-5-(2-(4-fluoro-3-(4-(methylsulfonyl)piperazin-1-yl)phenyl)cyclopropyl)-2,2′-bipyrimidine;
trans-5-(2-(3-(3,3-difluoropyrrolidin-1-yl)-4-fluorophenyl)cyclopropyl)-2,2′-bipyrimidine;
trans-5-(2-(4-chloro-3-fluoro-5-methoxyphenyl)cyclopropyl)-2,2′-bipyrimidine;
trans-5-(2-(4-fluoro-3-(3-methoxyazetidin-1-yl)phenyl)cyclopropyl)-2,2′-bipyrimidine;
trans-5-((2-(4-chloro-3-(cyclopropylmethoxy)-5-methylphenyl)cyclopropyl)-2,2′-bipyrimidine;
trans-5-(2-(4-chloro-5-methoxy-2-methylphenyl)cyclopropyl)-2,2′-bipyrimidine;
trans-1-(5-(2-([2,2′-bipyrimidin]-5-yl)cyclopropyl)-2-fluorophenyl)azetidin-3-ol;
trans-5-(2-(4-chloro-2-fluoro-3-methoxyphenyl)cyclopropyl)-2,2′-bipyrimidine;
trans-5-(2-(4-chloro-3-methoxy-5-methylphenyl)cyclopropyl)-2,2′-bipyrimidine;
trans-5-(2-(3,4-dichloro-5-methoxyphenyl)cyclopropyl)-2,2′-bipyrimidine;
trans-5-(2-(4-chloro-3-(cyclopropylmethoxy)-2-methylphenyl)cyclopropyl)-2,2′-bipyrimidine;
trans-5-(2-([2,2′-bipyrimidin]-5-yl)cyclopropyl)-2-fluoro-N,N-dimethylaniline;
trans-(3S)-1-(5-(2-([2,2′-bipyrimidin]-5-yl)cyclopropyl)-2-fluorophenyl)pyrrolidin-3-ol;
trans-5-(2-(4-fluoro-3-((S)-3-methoxypyrrolidin-1-yl)phenyl)cyclopropyl)-2,2′-bipyrimidine;
trans-(3R)-1-(5-(2-([2,2′-bipyrimidin]-5-yl)cyclopropyl)-2-fluorophenyl)pyrrolidin-3-ol;
trans-5-(2-(4-chloro-3-methoxy-2-methylphenyl)cyclopropyl)-2,2′-bipyrimidine;
trans-5-(2-(4-fluoro-3-methoxyphenyl)cyclopropyl)-4-isopropyl-2,2′-bipyrimidine;
trans-4-ethyl-5-(2-(4-fluoro-3-methoxyphenyl)cyclopropyl)-2,2′-bipyrimidine;
trans-4-cyclohexyl-5-(2-(4-fluoro-3-methoxyphenyl)cyclopropyl)-2,2′-bipyrimidine;
trans-5-(2-(4-fluoro-3-methoxyphenyl)cyclopropyl)-4-methoxy-2,2′-bipyrimidine;
trans-5-(2-(3-(azetidin-1-yl)-4-fluorophenyl)cyclopropyl)-2,2′-bipyrimidine;
trans-5-(2-(3,4-difluoro-5-methoxyphenyl)cyclopropyl)-2,2′-bipyrimidine;
trans-5-(2-(4-chloro-2-fluoro-5-methoxyphenyl)cyclopropyl)-2,2′-bipyrimidine;
trans-5-(2-(2,4-dichloro-5-methoxyphenyl)cyclopropyl)-2,2′-bipyrimidine;
trans-5-(2-(4-chloro-3-fluoro-5-(pyrrolidin-1-yl)phenyl)cyclopropyl)-2,2′-bipyrimidine;
trans-5-(2-(3,4-difluoro-5-(pyrrolidin-1-yl)phenyl)cyclopropyl)-2,2′-bipyrimidine;
trans-5-(2-(4-fluoro-3-((R)-3-methoxypyrrolidin-1-yl)phenyl)cyclopropyl)-2,2′-bipyrimidine;
trans-5-(2-(3,4-difluoro-5-((R)-3-methoxypyrrolidin-1-yl)phenyl)cyclopropyl)-2,2′-bipyrimidine;
trans-5-(2-(4-chloro-3,5-dimethoxyphenyl)cyclopropyl)-2,2′-bipyrimidine;
trans-5-(2-(4-fluoro-3-methoxy-5-(pyrrolidin-1-yl)phenyl)cyclopropyl)-2,2′-bipyrimidine;
trans-5-(2-(3,4-difluoro-5-((S)-3-methoxypyrrolidin-1-yl)phenyl)cyclopropyl)-2,2′-bipyrimidine;
trans-(3R)-1-(5-(2-([2,2′-bipyrimidin]-5-yl)cyclopropyl)-2,3-difluorophenyl)pyrrolidin-3-ol;
trans-(3S)-1-(5-(2-([2,2′-bipyrimidin]-5-yl)cyclopropyl)-2,3-difluorophenyl)pyrrolidin-3-ol;
trans-5-(2-(3-chloro-4-fluoro-5-(pyrrolidin-1-yl)phenyl)cyclopropyl)-2,2′-bipyrimidine;
trans-5-(2-(3-chloro-4-fluoro-5-((R)-3-methoxypyrrolidin-1-yl)phenyl)cyclopropyl)-2,2′-bipyrimidine;
trans-5-(2-(3-fluoro-5-methoxyphenyl)cyclopropyl)-2,2′-bipyrimidine;
trans-5-(2-(4-chloro-2,3-dimethoxyphenyl)cyclopropyl)-2,2′-bipyrimidine;
trans-5-(2-(3-chloro-4-fluoro-5-((S)-3-methoxypyrrolidin-1-yl)phenyl)cyclopropyl)-2,2′-bipyrimidine;
trans-5-(2-(3-chloro-5-fluorophenyl)cyclopropyl)-2,2′-bipyrimidine;
trans-(3S)-1-(5-(2-([2,2′-bipyrimidin]-5-yl)cyclopropyl)-3-chloro-2-fluorophenyl)pyrrolidin-3-ol;
trans-5-(2-(3,4-difluoro-5-(3-methoxyazetidin-1-yl)phenyl)cyclopropyl)-2,2′-bipyrimidine;
trans-2-(5-(2-(3,4-difluoro-5-methoxyphenyl)cyclopropyl)pyridin-2-yl)pyrimidine;
trans-(3R)-1-(5-(2-([2,2′-bipyrimidin]-5-yl)cyclopropyl)-3-chloro-2-fluorophenyl)pyrrolidin-3-ol;
trans-5-(2-(3-(cyclopropylmethoxy)-4,5-difluorophenyl)cyclopropyl)-2,2′-bipyrimidine;
trans-5-(2-(3,4-difluoro-5-(3-methoxypropoxy)phenyl)cyclopropyl)-2,2′-bipyrimidine;
trans-5-(2-(2,4-dichloro-3-methoxyphenyl)cyclopropyl)-2,2′-bipyrimidine;
trans-5-(2-(3,4-difluoro-5-(2-methoxyethoxy)phenyl)cyclopropyl)-2,2′-bipyrimidine;
trans-2-(5-(2-([2,2′-bipyrimidin]-5-yl)cyclopropyl)-2,3-difluorophenyl)-7-oxa-2-azaspiro[3.5]nonane;
trans-5-(2-(3-(3,3-dimethylazetidin-1-yl)-4,5-difluorophenyl)cyclopropyl)-2,2′-bipyrimidine;
trans-6-(5-(2-(4-fluoro-3-methoxyphenyl)cyclopropyl)-[2,2′-bipyrimidin]-4-yl)-2-methylbenzo[d]thiazole;
trans-5-(2-(4-Fluoro-3-methoxyphenyl)cyclopropyl)-4-phenyl-2,2′-bipyrimidine;
trans-5-(2-(4-fluoro-3-methoxyphenyl)cyclopropyl)-4-(piperidin-1-yl)-2,2′-bipyrimidine;
trans-5-(2-(4-fluoro-3,5-dimethoxyphenyl)cyclopropyl)-2,2′-bipyrimidine;
trans-5-(2-(3-chloro-4-fluoro-5-methoxyphenyl)cyclopropyl)-2,2′-bipyrimidine;
trans-5-(2-(4-fluoro-3-methoxy-5-methylphenyl)cyclopropyl)-2,2′-bipyrimidine;
trans-5-(2-(3-methoxy-4-(trifluoromethoxy)phenyl)cyclopropyl)-2,2′-bipyrimidine;
trans-5-(2-(3-methoxy-4-(trifluoromethyl)phenyl)cyclopropyl)-2,2′-bipyrimidine;
trans-5-(2-(5-chloro-4-fluoro-2-(3-methoxypropoxy)phenyl)cyclopropyl)-2,2′-bipyrimidine;
trans-5-(2-(5-chloro-4-fluoro-2-(2-methoxyethoxy)phenyl)cyclopropyl)-2,2′-bipyrimidine;
trans-5-(2-(5-chloro-4-methoxy-[1,1′-biphenyl]-2-yl)cyclopropyl)-2,2′-bipyrimidine;
trans-5-(2-(3,4,5-trifluorophenyl)cyclopropyl)-2,2′-bipyrimidine;
trans-5-(2-(3-methoxy-5-(trifluoromethyl)phenyl)cyclopropyl)-2,2′-bipyrimidine;
trans-5-(2-(3-methoxy-5-(trifluoromethoxy)phenyl)cyclopropyl)-2,2′-bipyrimidine;
trans-5-(2-(4-fluoro-3-methoxy-5-(trifluoromethyl)phenyl)cyclopropyl)-2,2′-bipyrimidine;
trans-5-(2-(naphthalen-1-yl)cyclopropyl)-2,2′-bipyrimidine;
trans-5-(2-(naphthalen-2-yl)cyclopropyl)-2,2′-bipyrimidine;
trans-5-(2-(4-fluoronaphthalen-1-yl)cyclopropyl)-2,2′-bipyrimidine;
trans-5-(2-(4-fluoronaphthalen-2-yl)cyclopropyl)-2,2′-bipyrimidine;
trans-3-(2-([2,2′-bipyrimidin]-5-yl)cyclopropyl)imidazo[1,2-a]pyridine;
trans-5-(2-([2,2′-bipyrimidin]-5-yl)cyclopropyl)quinoline;
trans-5-(2-([2,2′-bipyrimidin]-5-yl)cyclopropyl)-8-fluoroquinoline;
trans-5-(2-([2,2′-bipyrimidin]-5-yl)cyclopropyl)-8-methoxyquinoline;
trans-5-(2-(3,4-difluoro-5-methoxyphenyl)cyclopropyl)-2-(pyridin-2-yl)pyrimidine;
trans-5-(2-(4-fluoro-3,5-dimethoxyphenyl)cyclopropyl)-2-(pyridin-2-yl)pyrimidine;
trans-2-(pyridin-2-yl)-5-(2-(3,4,5-trimethoxyphenyl)cyclopropyl)pyrimidine;
trans-5-(2-(3,4-difluoro-5-methoxyphenyl)cyclopropyl)-2,4′-bipyrimidine;
trans-5-(2-(3,4-difluoro-5-methoxyphenyl)cyclopropyl)-2-(pyrazin-2-yl)pyrimidine;
trans-5-(2-(3,4-difluoro-5-methoxyphenyl)cyclopropyl)-2-(3-fluoropyridin-2-yl)pyrimidine;
trans-5-(2-(3,4-difluoro-5-methoxyphenyl)cyclopropyl)-2-(3-methoxypyridin-2-yl)pyrimidine;
trans-5-(2-(3,4-difluoro-5-(1H-imidazol-1-yl)phenyl)cyclopropyl)-2,2′-bipyrimidine;
trans-5-(2-(5,6-dimethoxypyridin-3-yl)cyclopropyl)-2,2′-bipyrimidine;
trans-5-(2-(3-(difluoromethoxy)-4-fluorophenyl)cyclopropyl)-2,2′-bipyrimidine;
trans-5-(2-(4-fluoro-3-(4-methylpiperazin-1-yl)phenyl)cyclopropyl)-2,2′-bipyrimidine;
trans-5-(2-(2,4-difluoro-3-methoxyphenyl)cyclopropyl)-2,2′-bipyrimidine;
trans-3-(2-(4-fluoro-3-methoxyphenyl)cyclopropyl)-6-(pyrimidin-2-yl)pyridazine;
trans-4-(2-(4-fluoro-3-methoxyphenyl)cyclopropyl)-1-(pyrimidin-2-yl)isoquinoline;
trans-5-(2-(4-fluoro-3-(piperidin-1-yl)phenyl)cyclopropyl)-2,2′-bipyrimidine;
trans-1-(5-(2-([2,2′-bipyrimidin]-5-yl)cyclopropyl)-2-fluorophenyl)pyrrolidin-2-one;
trans-5-(2-(4-chloro-3-(pyrrolidin-1-yl)phenyl)cyclopropyl)-2,2′-bipyrimidine;
trans-5-((2-([2,2′-bipyrimidin]-5-yl)cyclopropyl)-2-chloro-N-methylbenzamide;
trans-5-(2-([2,2′-bipyrimidin]-5-yl)cyclopropyl)-2-chloro-N,N-dimethylbenzamide;
trans-N-(5-(2-([2,2′-bipyrimidin]-5-yl)cyclopropyl)-2-chlorophenyl)acetamide;
trans-5-(2-([2,2′-bipyrimidin]-5-yl)cyclopropyl)-N-cyclopentyl-2,3-difluoroaniline;
trans-6-(5-(2-([2,2′-bipyrimidin]-5-yl)cyclopropyl)-2,3-difluorophenyl)-2-oxa-6-azaspiro[3.3]heptane;
trans-5-(2-(3,4-difluoro-5-(3-isopropoxyazetidin-1-yl)phenyl)cyclopropyl)-2,2′-bipyrimidine;
trans-5-(2-(3-(3-(tert-butyl sulfonyl)azetidin-1-yl)-4,5-difluorophenyl)cyclopropyl)-2,2′-bipyrimidine;
trans-5-(2-(3,4-difluoro-5-(3-(2-methoxyethoxy)azetidin-1-yl)phenyl)cyclopropyl)-2,2′-bipyrimidine;
trans-5-(2-(3-(3-(3,4-difluoro-5-methoxyphenyl)azetidin-1-yl)-4,5-difluorophenyl)cyclopropyl)-2,2′-bipyrimidine;
trans-5-(2-(3-(3-(3,4-difluorophenyl)azetidin-1-yl)-4,5-difluorophenyl)cyclopropyl)-2,2′-bipyrimidine;
trans-5-(2-(3,4-difluoro-5-(3-(4-fluoro-3-methoxyphenyl)azetidin-1-yl)phenyl)cyclopropyl)-2,2′-bipyrimidine;
trans-5-(2-(3-(3-(3,4-dimethoxyphenyl)azetidin-1-yl)-4,5-difluorophenyl)cyclopropyl)-2,2′-bipyrimidine;
trans-5-(2-(3,4-difluoro-5-((1-methyl-1H-1,2,4-triazol-3-yl)methoxy)phenyl)cyclopropyl)-2,2′-bipyrimidine;
trans-2-((5-(2-([2,2′-bipyrimidin]-5-yl)cyclopropyl)-2,3-difluorophenoxy)methyl)thiazole;
trans-5-(2-(3,4-difluoro-5-((1-methyl-1H-pyrazol-3-yl)methoxy)phenyl)cyclopropyl)-2,2′-bipyrimidine;
trans-5-(2-(3-(3,3-dimethylpyrrolidin-1-yl)-4,5-difluorophenyl)cyclopropyl)-2,2′-bipyrimidine;
trans-6-(5-(2-([2,2′-bipyrimidin]-5-yl)cyclopropyl)-2,3-difluorophenyl)-2-oxa-6-azaspiro[3.4]octane;
trans-1-((3S)-3-((5-(2-([2,2′-bipyrimidin]-5-yl)cyclopropyl)-2,3-difluorophenyl)amino)pyrrolidin-1-yl)ethenone;
trans-1-((3R)-3-((5-(2-([2,2′-bipyrimidin]-5-yl)cyclopropyl)-2,3-difluorophenyl) amino) pyrrolidin-1-yl)ethenone;
trans-(3S)-N-(5-(2-([2,2′-bipyrimidin]-5-yl)cyclopropyl)-2,3-difluorophenyl)-1-methylpyrrolidin-3-amine;
trans-(3R)-N-(5-(2-([2,2′-bipyrimidin]-5-yl)cyclopropyl)-2,3-difluorophenyl)-1-methylpyrrolidin-3-amine;
trans-5-(2-(3,4-difluoro-5-(4-methyl-1H-pyrazol-1-yl)phenyl)cyclopropyl)-2,2′-bipyrimidine;
trans-N-(5-(2-([2,2′-bipyrimidin]-5-yl)cyclopropyl)-2,3-difluorophenyl)-N-methyloxetan-3-amine;
trans-(1-(5-(2-([2,2′-bipyrimidin]-5-yl)cyclopropyl)-2,3-difluorophenyl)-1H-pyrazol-4-yl)methanol;
trans-5-(2-(3-((3R,4 S)-3,4-dimethoxypyrrolidin-1-yl)-4,5-difluorophenyl)cyclopropyl)-2,2′-bipyrimidine;
trans-5-(2-(3,4-difluoro-5-(4-(methoxymethyl)-1H-pyrazol-1-yl)phenyl)cyclopropyl)-2,2′-bipyrimidine;
trans-1-(5-(2-([2,2′-bipyrimidin]-5-yl)cyclopropyl)-2,3-difluorophenyl)-5,6-dimethoxy-1H-benzo[d]imidazole;
trans-2-(5-(2-([2,2′-bipyrimidin]-5-yl)cyclopropyl)-2,3-difluorophenyl)-2-azaspiro[3.3]heptan-6-ol;
trans-(3R,4R)-1-(5-(2-([2,2′-bipyrimidin]-5-yl)cyclopropyl)-2,3-difluorophenyl)pyrrolidine-3,4-diol;
trans-4-(3-(5-(2-([2,2′-bipyrimidin]-5-yl)cyclopropyl)-2,3-difluorophenoxy)propyl)morpholine;
trans-4-(3-(4-(2-([2,2′-bipyrimidin]-5-yl)cyclopropyl)-2,6-difluorophenoxy)propyl)morpholine;
trans-5-(2-(4((2-oxaspiro[3.3]heptan-6-yl)oxy)-3,5-difluorophenyl)cyclopropyl)-2,2′-bipyrimidine;
trans-5-(2-(3-((2-oxaspiro[3.3]heptan-6-yl)oxy)-4,5-difluorophenyl)cyclopropyl)-2,2′-bipyrimidine;
trans-2-((5-(2-([2,2′-bipyrimidin]-5-yl)cyclopropyl)-2,3-difluorophenyl)(methyl)amino)ethan-1-ol;
trans-5-(2-(3-(cyclopentyloxy)-4,5-difluorophenyl)cyclopropyl)-2,2′-bipyrimidine;
trans-5-(2-(3,4-difluoro-5-((R)-tetrahydrofuran-3-yl)oxy)phenyl)cyclopropyl)-2,2′-bipyrimidine;
trans-5-(2-(3,4-difluoro-5-((S)-tetrahydrofuran-3-yl)oxy)phenyl)cyclopropyl)-2,2′-bipyrimidine;
trans-5-(2-(3,4-difluoro-5-(4-methoxy-1H-pyrazol-1-yl)phenyl)cyclopropyl)-2,2′-bipyrimidine;
trans-5-(2-(3,4-difluoro-5-(1H-pyrazol-1-yl)phenyl)cyclopropyl)-2,2′-bipyrimidine;
trans-5-(2-(3,4-difluoro-5-(3-phenylazetidin-1-yl)phenyl)cyclopropyl)-2,2′-bipyrimidine;
trans-5-(2-(3-(3,4-difluoro-1H-pyrrol-1-yl)-4,5-difluorophenyl)cyclopropyl)-2,2′-bipyrimidine;
trans-1-(5-(2-([2,2′-bipyrimidin]-5-yl)cyclopropyl)-2,3-difluorophenyl)-1H-pyrazol-4-ol;
trans-5-(2-(3,4-difluoro-5-(4-methyl-1H-imidazol-1-yl)phenyl)cyclopropyl)-2,2′-bipyrimidine;
trans-ethyl 2-([2,2′-bipyrimidin]-5-yl)-3-(3,4-difluoro-5-methoxyphenyl)cyclopropane-1-carboxylate;
trans-4-(2-([2,2′-bipyrimidin]-5-yl)cyclopropyl)-6,7-difluoro-1-(3-methoxypropyl)-1H-indazole;
trans-6-(2-([2,2′-bipyrimidin]-5-yl)cyclopropyl)-4-fluoro-2-(3-methoxypropyl)-2H-indazole;
trans-2-(2-([2,2′-bipyrimidin]-5-yl)cyclopropyl)-4,6-difluorobenzo[d]thiazole;
trans-6-(2-([2,2′-bipyrimidin]-5-yl)cyclopropyl)-4-fluoro-1-isopropyl-2-methyl-1H-benzo[d]imidazole;
trans-6-(2-([2,2′-bipyrimidin]-5-yl)cyclopropyl)-4-fluoro-2-methylbenzo[d]thiazole;
trans-5-(2-([2,2′-bipyrimidin]-5-yl)cyclopropyl)-7-fluoro-1-(3-methoxypropyl)-1H-benzo[d]imidazole;
trans-6-(2-([2,2′-bipyrimidin]-5-yl)cyclopropyl)-4-fluoro-1-(3-methoxypropyl)-1H-benzo[d]imidazole;
trans-6-(2-([2,2′-bipyrimidin]-5-yl)cyclopropyl)-4-fluoro-1-(3-methoxypropyl)-1H-indazole;
trans-4-(2-([2,2′-bipyrimidin]-5-yl)cyclopropyl)-7-fluoro-1-(3-methoxypropyl)-1H-indazole;
trans-4-(2-([2,2′-bipyrimidin]-5-yl)cyclopropyl)-7-fluoro-2-(3-methoxypropyl)-2H-indazole; and
trans-5-(2-([2,2′-bipyrimidin]-5-yl)cyclopropyl)-2-methylbenzo[d]thiazole.

22. A pharmaceutical composition comprising at least one compound of claim 1 and at least one pharmaceutically acceptable carrier.

23. The pharmaceutical composition of claim 22, further comprising at least one additional agent useful for treating hepatitis infection.

24. The pharmaceutical composition of claim 23, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; and GalNAc-siRNA conjugate targeted against an HBV gene transcript.

25. The pharmaceutical composition of claim 24, wherein the immunostimulator is a checkpoint inhibitor.

26. The pharmaceutical composition of claim 25, wherein the checkpoint inhibitor is a PD-L1 inhibitor.

27. The pharmaceutical composition of claim 23, wherein the hepatitis virus is at least one selected from the group consisting of hepatitis B virus (HBV) and hepatitis D virus (HDV).

28. A method of treating, ameliorating, and/or preventing hepatitis B virus (HBV) infection in a subject, the method comprising administering to the subject in need thereof a therapeutically effective amount of the compound of claim 1 and/or the pharmaceutical composition of claim 22, or a salt, solvate, stereoisomer, tautomer, or any mixtures thereof.

29. (canceled)

30. The method of claim 28, wherein the subject is further infected with hepatitis D virus (HDV).

31. (canceled)

32. The method of claim 28, wherein the subject is further administered at least one additional agent useful for treating the hepatitis virus infection.

33. The method of claim 32, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; and GalNAc-siRNA conjugate targeted against an HBV gene transcript.

34. The method of claim 33, wherein the immunostimulator is a checkpoint inhibitor, optionally wherein the checkpoint inhibitor is a PD-L1 inhibitor.

35. (canceled)

36. The method of claim 32, wherein the subject is co-administered the at least one compound and the at least one additional agent, optionally wherein the at least one compound and the at least one additional agent are coformulated.

37. (canceled)

38. The method of claim 28, wherein the subject is a mammal optionally wherein the mammal is human.

39. (canceled)
